(19) United States
(12) Patent Application Publication
(10) Pub. No.: US 2011/0130380 A1
(43) Pub. Date: Jun. 2, 2011

(54) Heteroaryl Kinase Inhibitors

(76) Inventors: Paul A. Barsanti, Pleasant Hill, CA (US); Cheng Hu, Menlo Park, CA (US); Xianming Jin, San Ramon, CA (US); Simon C. Ng, Walnut Creek, CA (US); Keith B. Pfister, San Ramon, CA (US); Martin Sendzik, San Mateo, CA (US); James Sutton, Pleasanton, CA (US)

(21) Appl. No.: 12/874,389

(22) Filed: Sep. 2, 2010

Related U.S. Application Data
(60) Provisional application No. 61/275,938, filed on Sep. 4, 2009.
(60) Provisional application No. 61/284,961, filed on Dec. 28, 2009.

(51) Int. Cl. A61K 31/444; C07D 401/14; A61K 31/4545; C07D 401/04; C07D 405/14; C07D 409/14; C07D 413/14; A61K 31/5377; A61K 31/553; A61K 31/55; A61P 29/00; A61P 37/06; A61P 19/08; A61P 3/00; A61P 25/28; A61P 35/00; A61P 9/00; A61P 11/06; A61P 31/12

(52) U.S. Cl. 514/210.18; 546/194; 514/318; 546/257; 514/334; 546/256; 514/333; 544/131; 514/237.2; 546/187; 514/316; 540/544; 514/211.15; 540/597; 514/217.04

(57) ABSTRACT

The present invention provides compounds of Formula (I):

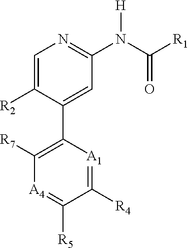

and pharmaceutically acceptable salts thereof. Also provided is a method of using a compound of Formula I for treating a disease or condition mediated by a CDK inhibitor.

### CROSS-REFERENCE TO RELATED APPLICATIONS

This application claims the benefit of priority under 35 U.S.C. §119(e) to U.S. provisional application Ser. No. 61/275,938, filed on Sep. 4, 2009, and U.S. provisional application Ser. No. 61/284,961 filed on Dec. 28, 2009, which are incorporated herein by reference in their entirety.

### FIELD OF THE INVENTION

The invention provides a novel class of compounds, pharmaceutical compositions comprising such compounds and methods of using such compounds to treat or prevent diseases or disorders associated with aberrant cellular signaling pathways that can be modulated by inhibition of kinases, particularly diseases or disorders that involve aberrant cellular signaling pathways that can be modulated by inhibition of CDK9.

### BACKGROUND

Protein kinases constitute a large family of structurally related enzymes that are responsible for the control of a variety of signal transduction processes within the cell. (Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

Many diseases are associated with abnormal cellular responses triggered by the protein kinase-mediated events described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, viral diseases, and hormone-related diseases. Accordingly, there has been a substantial effort in medicinal chemistry to find protein kinase inhibitors that are effective as therapeutic agents.

The cyclin-dependent kinase (CDK) complexes are a class of kinases that are targets of interest. These complexes comprise at least a catalytic (the CDK itself) and a regulatory (cyclin) subunit. Some of the more important complexes for cell cycle regulation include cyclin A (CDK1—also known as cdc2, and CDK2), cyclin B1-B3 (CDK1) and cyclin D1-D3 (CDK2, CDK4, CDK5, CDK6), cyclin E (CDK2). Each of these complexes is involved in a particular phase of the cell cycle. Additionally, CDKs 7, 8, and 9 are implicated in the regulation of transcription.

The CDKs seem to participate in cell cycle progression and cellular transcription, and loss of growth control is linked to abnormal cell proliferation in disease (see e.g., Malumbres and Barbacid, Nat. Rev. Cancer 2001, 1:222). Increased activity or temporally abnormal activation of cyclin-dependent kinases has been shown to result in the development of human tumors (Sherr C. J., Science 1996, 274: 1672-1677). Indeed, human tumor development is commonly associated with alterations in either the CDK proteins themselves or their regulators (Cordon-Cardo C., Am. J. Pat1/701. 1995; 147: 545-560; Karp J. E. and Broder S., Nat. Med. 1995; 1: 309-320; Hall M. et al., Adv. Cancer Res. 1996; 68: 67-108).

CDKs 7 and 9 seem to play key roles in transcription initiation and elongation, respectively (see, e.g., Peterlin and Price. Cell 23: 297-305, 2006, Shapiro. J. Clin. Oncol. 24: 1770-83, 2006). Inhibition of CDK9 has been linked to direct induction of apoptosis in tumor cells of hematopoetic lineages through down-regulation of transcription of antiapoptotic proteins such as Mcl1 (Chao, S.-H. et al. J. Biol. Chem. 2000; 275:28345-28348; Chao, S.-H. et al. J. Biol. Chem. 2001; 276:31793-31799; Lam et. al. Genome Biology 2: 0041.1-11, 2001; Chen et al. Blood 2005; 106:2513; MacCallum et al. Cancer Res. 2005; 65:5399; and Alvi et al. Blood 2005; 105:4484). In solid tumor cells, transcriptional inhibition by downregulation of CDK9 activity synergizes with inhibition of cell cycle CDKs, for example CDK1 and 2, to induce apoptosis (Cai, D.-P., Cancer Res 2006, 66:9270. Inhibition of transcription through CDK9 or CDK7 may have selective non-proliferative effect on the tumor cell types that are dependent on the transcription of mRNAs with short half lives, for example Cyclin D1 in Mantle Cell Lymphoma. Some transcription factors such as Myc and NF-kB selectively recruit CDK9 to their promoters, and tumors dependent on activation of these signaling pathways may be sensitive to CDK9 inhibition.

Small molecule CDK inhibitors may also be used in the treatment of cardiovascular disorders such as restenosis and atherosclerosis and other vascular disorders that are due to aberrant cell proliferation. Vascular smooth muscle proliferation and intimal hyperplasia following balloon angioplasty are inhibited by over-expression of the cyclin-dependent kinase inhibitor protein. Moreover, the purine CDK2 inhibitor CVT-313 (Ki=95 nM) resulted in greater than 80% inhibition of neointima formation in rats.

CDKs are important in neutrophil-mediated inflammation and CDK inhibitors promote the resolution of inflammation in animal models. (Rossi, A. G. et al, Nature Med. 2006, 12:1056). Thus CDK inhibitors, including CDK9 inhibitors, may act as anti-inflammatory agents.

Certain CDK inhibitors are useful as chemoprotective agents through their ability to inhibit cell cycle progression of normal untransformed cells (Chen, et al. J. Natl. Cancer Institute, 2000; 92: 1999-2008). Pre-treatment of a cancer patient with a CDK inhibitor prior to the use of cytotoxic agents can reduce the side effects commonly associated with chemotherapy. Normal proliferating tissues are protected from the cytotoxic effects by the action of the selective CDK inhibitor.

Accordingly, there is a great need to develop inhibitors of protein kinases, such as CDK1, CDK2, CDK3, CDK4, CDK5, CDK6, CDK7, CDK8 and CDK9, as well as combinations thereof.

### SUMMARY

The present invention provides a compound of Formula I

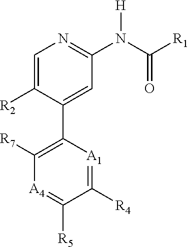

or a pharmaceutically acceptable salt thereof, wherein:

R₁ is C₁₋₈ alkyl, C₃₋₈ cycloalkyl, C₃₋₈ branched alkyl, —(CH₂)₀₋₃—O—C₁₋₄ alkyl, —(CH₂)₀₋₂ heteroaryl, or a 4 to 8 membered heterocycloalkyl group, wherein said groups are each independently optionally substituted with one to three substituents selected from the group consisting of —NH₂, —OH, ═O, —C₁₋₄ alkyl, —C₁₋₄ haloalkyl, —C₃₋₅ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, —O—(CH₂)₂₋₃—O—C₁₋₂ alkyl, —O—C₁₋₄ alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;

R₂ is hydrogen, C₁₋₄ alkoxy, C₁₋₄ haloalkyl, C₁₋₄-alkyl, or halogen;

A₁ is N or CR₃;

A₄ is N or CR₆, with the proviso that only one of A₁ and A₄ is a N;

R₃ is C₁₋₄ alkyl, H, or OC₁₋₄ alkyl;

R₄ is hydrogen, halogen, 5 to 7 membered heterocyclyl-aryl, or A₆-L-R₉;

R₅ is hydrogen, C₁₋₄ alkyl, or halogen;

R₆ is hydrogen, C₁₋₄ alkyl, or halogen;

R₇ is hydrogen, C₁₋₄ alkyl, or halogen;

A₆ is NR₈;

L is C₀₋₃-alkylene or C₃₋₈ branched alkylene;

R₈ is hydrogen, C₁₋₄ alkyl; or —C₃₋₈ branched alkyl; and

R₉ is hydrogen, C₁₋₆ alkyl, C₃₋₈ cycloalkyl, 4 to 8 member heterocycloalkyl, aryl, or heteroaryl, wherein said groups are optionally substituted with one to three substituents each independently selected from hydrogen, halogen, C₁₋₄ alkyl, C₁₋₄ haloalkyl, —OH, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl.
Another embodiment of the present invention provides a compound of Formula I, or a pharmaceutically acceptable salt thereof, wherein:

R₁ is C₁₋₈ alkyl, C₃₋₈ cycloalkyl, C₃₋₈ branched alkyl, —(CH₂)₀₋₃—O—C₁₋₄ alkyl, —(CH₂)₀₋₂ heteroaryl, or a 4 to 8 membered heterocycloalkyl group, wherein said groups are each independently optionally substituted with one to three substituents selected from the group consisting of —NH₂, —OH, ═O, —C₁₋₄ alkyl, —C₁₋₄ haloalkyl, —C₃₋₅ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, —O—(CH₂)₂₋₃—O—C₁₋₂ alkyl, —O—C₁₋₄ alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;

R₂ is hydrogen, C₁₋₄ alkoxy, C₁₋₄ haloalkyl, C₁₋₄-alkyl, or halogen;

A₁ is N;

A₄ is N or CR₆;

R₄ is hydrogen, halogen, 5 to 7 membered heterocyclyl-aryl, or A₆-L-R₉;

R₅ is hydrogen, C₁₋₄ alkyl, or halogen;

R₆ is hydrogen, C₁₋₄ alkyl, or halogen;

R₇ is hydrogen, C₁₋₄ alkyl, or halogen;

A₆ is NR₈;

L is C₀₋₃-alkylene or C₃₋₈ branched alkylene;

R₈ is hydrogen, C₁₋₄ alkyl; or —C₃₋₈ branched alkyl; and

R₉ is hydrogen, C₁₋₆ alkyl, C₃₋₈ cycloalkyl, 4 to 8 member heterocycloalkyl, aryl, or heteroaryl, wherein said groups are optionally substituted with one to three substituents each independently selected from hydrogen, halogen, C₁₋₄ alkyl, C₁₋₄ haloalkyl, —OH, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl.

Yet another embodiment provides a compound of Formula I, or a pharmaceutically acceptable salt thereof, wherein:

R₁ is C₁₋₈ alkyl, C₃₋₈ cycloalkyl, C₃₋₈ branched alkyl, —(CH₂)₀₋₃—O—C₁₋₄ alkyl, —(CH₂)₀₋₂ heteroaryl, or a 4 to 8 membered heterocycloalkyl group, wherein said groups are each independently optionally substituted with one to three substituents selected from the group consisting of —NH₂, —OH, ═O, —C₁₋₄ alkyl, —C₁₋₄ haloalkyl, —C₃₋₅ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, —O—(CH₂)₂₋₃—O—C₁₋₂ alkyl, —O—C₁₋₄ alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;

R₂ is hydrogen, C₁₋₄ alkoxy, C₁₋₄ haloalkyl, C₁₋₄-alkyl, or halogen;

A₁ is CR₃;

A₄ is N;

R₃ is C₁₋₄ alkyl, H, or OC₁₋₄ alkyl;

R₄ is hydrogen, halogen, 5 to 7 membered heterocyclyl-aryl, or A₆-L-R₉;

R₅ is hydrogen, C₁₋₄ alkyl, or halogen;

R₇ is hydrogen, C₁₋₄ alkyl, or halogen;

A₆ is NR₈;

L is C₀₋₃-alkylene or C₃₋₈ branched alkylene;

R₈ is hydrogen, C₁₋₄ alkyl; or —C₃₋₈ branched alkyl; and

R₉ is hydrogen, C₁₋₆ alkyl, C₃₋₈ cycloalkyl, 4 to 8 member heterocycloalkyl, aryl, or heteroaryl, wherein said groups are optionally substituted with one to three substituents each independently selected from hydrogen, halogen, C₁₋₄ alkyl, C₁₋₄ haloalkyl, —OH, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl.

The present invention also provides a compound of Formula I

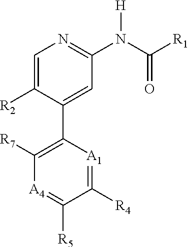

or a pharmaceutically acceptable salt thereof, wherein:

R₁ is C₃₋₈ cycloalkyl, —(CH₂)₁₋₂ heteroaryl, or a 4 to 8 membered heterocycloalkyl group, wherein said cycloalkyl, heteroaryl, and heterocycloalkyl groups are each independently optionally substituted with one to three substituents selected from the group consisting of —NH—C(O)—CH₂—O—C₁₋₄ alkyl, —NHC(O)—C₁₋₄ alkyl, —C(O)—O—C₁₋₄alkyl, —C(O)—CH₂—O—C₁₋₄ alkyl, C₁₋₄ alkyl, —(CH₂)₁₋₃—O—C₁₋₂ alkyl, NH₂, —SO₂—C₁₋₄ alkyl, —NH—C(O)—C₁₋₄ alkyl, and —NH—SO₂—C₁₋₄ alkyl;

R₂ is C₁₋₄ alkoxy, or halogen;

A₁ is N or CR₃;

A₄ is N and CR₆, with the proviso that at least one of A₁ and A₄ is a N;

R₃ is halogen, C₁₋₄ alkoxy, or hydrogen;

R₄ is hydrogen, halogen, or A₆-L-R₉;

R₅ is hydrogen, C₁₋₄ alkyl, or halogen;

R₆ is hydrogen, or halogen;

R₇ is hydrogen, C₁₋₄ alkyl, or halogen;

A₆ is NR₈;

L is C₁₋₃-alkylene or C₃₋₈ branched alkylene;

R₈ is hydrogen, or C₁₋₄ alkyl; and

R₉ is hydrogen, 4 to 8 member heterocycloalkyl, heteroaryl, or aryl, wherein the heterocycloalkyl, heteroaryl, and aryl groups are optionally substituted with one to three substituents each independently selected from halogen, C₁₋₄ alkyl, or C₁₋₄ haloalkyl.

A preferred embodiment provides a compound of Formula I

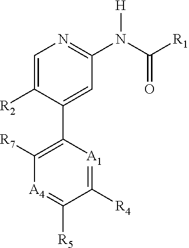

or a pharmaceutically acceptable salt thereof, wherein:

R₁ is C₃₋₈ cycloalkyl, —(CH₂)₁₋₂ heteroaryl, or a 4 to 8 membered heterocycloalkyl group, wherein said cycloalkyl, heteroaryl, and heterocycloalkyl groups are optionally substituted with one to three substituents each independently selected from —NH—C(O)—CH₂—O—C₁₋₄ alkyl, —NHC(O)—C₁₋₄ alkyl, —C(O)—O—C₁₋₄alkyl, —C(O)—CH₂—O—C₁₋₄ alkyl, C₁₋₄ alkyl, —(CH₂)₁₋₃—O—C₁₋₂ alkyl, NH₂, —SO₂—C₁₋₄ alkyl, —NH—C(O)—C₁₋₄ alkyl, or —NH—SO₂—C₁₋₄ alkyl;

R₂ is C₁₋₄ alkoxy, or halogen;

A₁ is N;

A₄ is CR₆;

R₄ is hydrogen, halogen, or A₆-L-R₉;

R₅ is hydrogen, C₁₋₄ alkyl, or halogen;

R₆ is hydrogen, or halogen;

R₇ is hydrogen, C₁₋₄ alkyl, or halogen;

A₆ is NR₈;

L is C₁₋₃-alkylene or C₃₋₈ branched alkylene;

R₈ is hydrogen, or C₁₋₄ alkyl; and

R₉ is hydrogen, 4 to 8 member heterocycloalkyl, heteroaryl, or aryl, wherein the heterocycloalkyl, heteroaryl, and aryl groups are optionally substituted with one to three substituents each independently selected from halogen, C₁₋₄ alkyl, or C₁₋₄ haloalkyl.

A further preferred embodiment provides a compound of Formula I, wherein:

R₁ is cyclohexyl or piperidinyl wherein said cyclohexyl and said piperidinyl are each optionally substituted with one to two substituents each independently selected from a group consisting of —NHC(O)—C₁₋₄ alkyl, —C(O)—O—C₁₋₄alkyl, —C(O)—CH₂—O—C₁₋₄ alkyl,

—C₁₋₄ alkyl, —(CH₂)₁₋₃—O—C₁₋₂ alkyl, —SO₂—C₁₋₄ alkyl, —NH—C(O)—C₁₋₄ alkyl, and —NH—SO₂—C₁₋₄ alkyl;

R₂ is halogen;

R₄ is hydrogen, or A₆-L-R₉;

R₅ is methyl, hydrogen, or halogen;

R₆ is —OCH₃, hydrogen, or halogen;

R₇ is hydrogen, or halogen;

A₆ is NR₈;

L is —CH₂— or C₃₋₆ branched alkylene;

R₈ is methyl or hydrogen; and

R₉ is tetrahydropyran, or phenyl, wherein said tetrahydropyran and phenyl groups are optionally substituted with one to two substituents each independently selected from halogen, or C₁₋₂-alkyl.

Provided in another preferred embodiment is a compound of Formula I

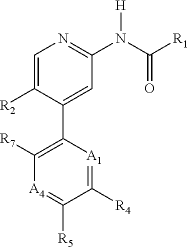

or a pharmaceutically acceptable salt thereof, wherein:

R₁ represents C₃₋₈ cycloalkyl, —(CH₂)₁₋₂ heteroaryl, or a 4 to 8 membered heterocycloalkyl group, wherein said cycloalkyl, heteroaryl, and heterocycloalkyl groups are optionally substituted with one to three substituents each independently selected from —NH—C(O)—CH₂—O—C₁₋₄ alkyl, —NHC(O)—C₁₋₄ alkyl, —C(O)—O—C₁₋₄alkyl, —C(O)—CH₂—O—C₁₋₄ alkyl, C₁₋₄ alkyl, —(CH₂)₁₋₃—O—C₁₋₂ alkyl, NH₂, —SO₂—C₁₋₄ alkyl, —NH—C(O)—C₁₋₄ alkyl, and

—NH—SO₂—C₁₋₄ alkyl;

R₂ is C₁₋₄ alkoxy, or halogen;

A₁ is CR₃;

A₄ is N;

R₃ is halogen, C₁₋₄ alkoxy, or hydrogen;

R₄ is hydrogen, halogen, or A₆-L-R₉;

R₅ is hydrogen, C₁₋₄ alkyl, or halogen;

R₇ is hydrogen, C₁₋₄ alkyl, or halogen;

A₆ is NR₈;

L is C₁₋₃-alkylene or C₃₋₈ branched alkylene;

R₈ is hydrogen, or C₁₋₄ alkyl;

R₉ is hydrogen, 4 to 8 member heterocycloalkyl, heteroaryl, or aryl, wherein the heterocycloalkyl, heteroaryl, and aryl groups are optionally substituted with one to three substituents each independently selected from halogen, C₁₋₄ alkyl, or C₁₋₄ haloalkyl.

Another preferred embodiment provides compounds of Formula I, wherein:

R₁ is cyclohexyl or piperidinyl wherein said cyclohexyl and said piperidinyl are each optionally substituted with one to two substituents selected from a group consisting of —NHC(O)—C₁₋₄ alkyl, —C(O)—O—C₁₋₄alkyl, —C(O)—CH₂—O—C₁₋₄ alkyl, —C₁₋₄ alkyl, —(CH₂)₁₋₃—O—C₁₋₂ alkyl, —SO₂—C₁₋₄ alkyl, —NH—C(O)—C₁₋₄ alkyl, and —NH—SO₂—C₁₋₄ alkyl;

R₂ is halogen;

R₄ is hydrogen or A₆-L-R₉;

R₅ is methyl, hydrogen, or halogen;

R₆ is hydrogen or halogen;

R₇ is hydrogen or halogen;

A₆ is NR₈;

L is —CH₂— or C₃₋₆ branched alkylene;

R₈ is methyl or hydrogen; and

R₉ is tetrahydropyran, or phenyl, wherein said tetrahydropyran and phenyl groups are optionally substituted with one to two substituents each independently halogen, or C₁₋₂-alkyl.

Another embodiment of the present invention provides a compound of Formula I:

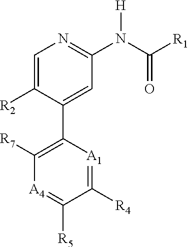

or a pharmaceutically acceptable salt thereof, wherein:

R₁ is selected from —(CH₂)₀₋₂-heteroaryl, —(CH₂)₀₋₂-aryl, C₁₋₈ alkyl, C₃₋₈ branched alkyl, C₃₋₈ cycloalkyl, and a 4 to 8 membered heterocycloalkyl group, wherein said groups are each independently optionally substituted;

R₂ is selected from hydrogen, C₁₋₄ alkoxy, C₁₋₄ haloalkyl, C₁₋₄-alkyl, and halogen;

A₁ is N;

A₄ is CR₆;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, hydroxyl, CN, —O—C₁₋₄ alkyl, —O—C₁₋₄ haloalkyl, C₃₋₄ cycloalkyl, C₃₋₄ cyclo haloalkyl, and halogen;

R₆ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;

R₇ is selected from hydrogen, C₁₋₄ alkyl, C₁₋₄ haloalkyl, O—C₁₋₃ alkyl, and halogen;

A₆ is selected from O, SO₂, and NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;

R₈ is selected from hydrogen, C₁₋₄ alkyl, and C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted; and

R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl.

A preferred embodiment provides a compound of Formula I, wherein:

R₁ is selected from —(CH₂)₀₋₂-heteroaryl, —(CH₂)₀₋₂-aryl, wherein said groups are each independently optionally substituted with one to three substituents selected from —NH₂, —F, —Cl, —OH, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ cyclo haloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;

R₂ is selected from hydrogen, C₁₋₄ alkoxy, C₁₋₄ haloalkyl, C₁₋₄-alkyl, and halogen;

A₁ is N;

A₄ is CR₆;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;

R₆ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;

R₇ is selected from hydrogen, C₁₋₄ alkyl, C₁₋₄ haloalkyl, O—C₁₋₃ alkyl, and halogen;

A₆ is O, SO₂, or NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, and C₃₋₈ branched alkylene;

R₈ is selected from hydrogen, C₁₋₄ alkyl, and C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;

R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

Another preferred embodiment provides a compound of Formula I, wherein:

R₁ is selected from —(CH₂)₀₋₂-heteroaryl, and —(CH₂)₀₋₂-aryl, wherein said groups are each independently optionally substituted with one to three substituents selected from the group consisting of —NH₂, F, Cl, —OH, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, —O—(CH₂)₁₋₂—C₃₋₆ cycloalkyl-R¹⁴, —O—(CH₂)₁₋₂—C₄₋₆ heterocycloalkyl-R¹⁴, —SO₂—NR¹⁵R¹⁶, and —SO₂—C₃₋₅ cycloalkyl;

R₂ is selected from hydrogen, and halogen;

A₁ is N;

A₄ is CR₆;

R₄ is selected from piperidinyl, morpholinyl, pyrrolidinyl, and A₆-L-R₉; wherein each said piperidinyl, morpholinyl, pyrrolidinyl group is substituted with R¹⁴;

R₅ is selected from hydrogen, Cl, F, and CF₃;

R₆ is hydrogen;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and C₁₋₄ alkyl;

R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl;

R¹⁴ is selected from phenyl, halogen, hydroxyl, C₁₋₂-alkyl, CF₃, and hydrogen; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

Yet another preferred embodiment provides a compound of Formula I, wherein:

R₁ is selected from C₁₋₈ alkyl, C₃₋₈ cycloalkyl, C₃₋₈ branched alkyl, and a 4 to 8 membered heterocycloalkyl group, wherein said groups are each independently optionally substituted with one to three substituents selected from —NH₂, —F, —OH, ═O, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ halo-cycloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl; —C(O)—NR¹⁵R¹⁶, and —SO₂—NR¹⁵R¹⁶, and further wherein, any two said substituents along with the atoms to which they are attached can form a ring;

R₂ is selected from hydrogen, C₁₋₄ alkoxy, C₁₋₄ haloalkyl, C₁₋₄-alkyl, and halogen;

A₁ is N;

A₄ is CR₆;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;

R₆ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is selected from O, SO₂, and NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;

R₈ is selected from hydrogen, C₁₋₄ alkyl, and C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;

R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

A further preferred embodiment provides a compound of Formula I, wherein:

R₁ is selected from C₁₋₈ alkyl, C₃₋₈ branched alkyl, C₃₋₈ cycloalkyl, and a 4 to 8 membered heterocycloalkyl group, wherein said groups are each independently optionally substituted with one to three substituents selected from the group consisting of —NH₂, F, —OH, ═O, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;

R₂ is selected from hydrogen, and halogen;

A₁ is N;

A₄ is CR₆;

R₄ is selected from piperidinyl, morpholinyl, pyrrolidinyl, and A₆-L-R₉; wherein each said piperidinyl, morpholinyl, pyrrolidinyl group is substituted with R¹⁴;

R₅ is selected from hydrogen, Cl, F, and CF₃;

R₆ is hydrogen;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and C₁₋₄ alkyl;

R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl; and

R¹⁴ is selected from phenyl, halogen, hydroxy, C₁₋₂-alkyl, and hydrogen.

Provided in yet another preferred embodiment is a compound of Formula I, wherein:

R₁ is selected from piperidinyl, morpholinyl, 1-methylpiperidinyl, tetrahydro-pyran, pyrrolidinyl, tetrahydro-furan, azetidine, pyrrolidin-2-one, azepane, and 1,4-oxazepane, wherein said R₁ groups are each independently optionally substituted with one to three substituents selected from F, OH, NH₂, CO-methyl, —NH-methyl, ethyl, fluoro-ethyl, trifluoro-ethyl, (CH₂)₂-methoxy, SO₂—CH₃, COO—CH₃, SO₂-ethyl, SO₂-cyclopropyl, methyl, SO₂—CH—(CH₃)₂, NH—SO₂—CH₃, NH—SO₂—C₂H₅, ═O, CF₃, (CH₂)-methoxy, methoxy, NH—SO₂—CH—(CH₃)₂, —(CH₂)—O—(CH₂)₂-methoxy, —O—CH—(CH₃)₂;

R₂ is selected from Cl, and F;

A₁ is N;

A₄ is CR₆;

R₄ is A₆-L-R₉;

R₅ is selected from Cl, F, and hydrogen;

R₆ is H;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and methyl; and

R₉ is selected from C₁₋₃ alkyl, C₄₋₆ branched alkyl, —(CH₂)₁₋₃—O—C₁₋₄ alkyl, —(CH₂)-pyridyl, benzyl, CD₂-tetrahydro-pyran, tetrahydro-pyran, tetrahydro-thiopyran 1,1-dioxide, piperidinyl, pyrrolidine-2-one, dioxane, cyclopropyl, tetrahydrofuran, cyclohexyl, and cycloheptyl, wherein said groups are optionally substituted with one to three substituents each independently selected from F, OCHF₂, CO-methyl, OH, methyl, methoxy, CN, ethyl, and NH—CO-methyl.

A particularly preferred embodiment provides a compound of Formula I, wherein:

R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, and —O—CH—(CH₃)₂;

R₂ is Cl;

R₄ is A₆-L-R₉;

R₅ is selected from Cl, F, and hydrogen;

R₆ is H;

R₇ is selected from Cl, F, and hydrogen;

A₆ is NR₈;

L is selected from —CH₂—, and —CD₂-;

R₈ is selected from hydrogen, and methyl; and

R₉ is selected from pyridyl, benzyl, tetrahydro-pyran, dioxane, and tetrahydrofuran, wherein said groups are optionally substituted with one to three substituents each independently selected from F, OH, methyl, ethyl, methoxy, and CN.

Provided in yet another particularly preferred embodiment is a compound of Formula I selected from:
((1R,3S)-3-{3,5′-Dichloro-6-[(tetrahydro-pyran-4-ylmethyl)-amino]-[2,4′]bipyridinyl-2′-ylcarbamoyl}-cyclopentyl)-carbamic acid methyl ester;
(1S,3R)-3-(Propane-2-sulfonylamino)-cyclopentanecarboxylic acid {3,5′-dichloro-6-[(tetrahydro-pyran-4-ylmethyl)-amino]-[2,4′]bipyridinyl-2′-yl}-amide;
(S)-3-{5′-Chloro-6-[(1′,1′-dioxo-hexahydro-1-thiopyran-4-ylmethyl)-amino]-[2,4′]bipyridinyl-2′-ylcarbamoyl}-piperidine-1-carboxylic acid methyl ester;
(S)-3-{3,5′-Dichloro-6-[(2,2-dimethyl-tetrahydro-pyran-4-ylmethyl)-amino]-[2,4′]bipyridinyl-2′-ylcarbamoyl}-piperidine-1-carboxylic acid methyl ester;
((1S,3R)-3-{3,5′-Dichloro-6-[(tetrahydro-pyran-4-ylmethyl)-amino]-[2,4′]bipyridinyl-2′-ylcarbamoyl}-cyclopentyl)-carbamic acid methyl ester;
(S)-1-Methanesulfonyl-piperidine-3-carboxylic acid {3,5′-dichloro-6-[(tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[((S)-2,2-dimethyl-tetrahydro-pyran-4-ylmethyl)-amino]-[2,4′]bipyridinyl-2′-yl}-amide;
(1R,3S)-3-Methanesulfonylamino-cyclopentanecarboxylic acid {3,5′-dichloro-6-[(tetrahydro-pyran-4-ylmethyl)-amino]-[2,4′]bipyridinyl-2′-yl}-amide;
(1S,3R)-3-Ethanesulfonylamino-cyclopentanecarboxylic acid {3,5′-dichloro-6-[(tetrahydro-pyran-4-ylmethyl)-amino]-[2,4′]bipyridinyl-2′-yl}-amide;
(S)-1-Ethanesulfonyl-piperidine-3-carboxylic acid {3,5′-dichloro-6-[(tetrahydro-pyran-4-ylmethyl)-amino]-[2,4′]bipyridinyl-2′-yl}-amide;
(S)-3-{3,5′-Dichloro-6-[((R)-2,2-dimethyl-tetrahydro-pyran-4-ylmethyl)-amino]-[2,4′]bipyridinyl-2′-ylcarbamoyl}-piperidine-1-carboxylic acid methyl ester;
(S)-1-Methanesulfonyl-piperidine-3-carboxylic acid {5′-chloro-6-[(tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[(2,2-dimethyl-tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[((R)-2,2-dimethyl-tetrahydro-pyran-4-ylmethyl)-amino]-[2,4′]bipyridinyl-2′-yl}-amide;
(1S,3R)-3-Methanesulfonylamino-cyclopentanecarboxylic acid {3,5′-dichloro-6-[(tetrahydro-pyran-4-ylmethyl)-amino]-[2,4′]bipyridinyl-2′-yl}-amide;
(S)-1-Ethanesulfonyl-piperidine-3-carboxylic acid {5′-chloro-6-[(tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[(tetrahydro-pyran-4-ylmethyl)-amino]-[2,4′]bipyridinyl-2′-yl}-amide;
(R)-Piperidine-3-carboxylic acid {5′-chloro-6-[((2R,6S)-2,6-dimethyl-tetrahydro-pyran-4-ylmethyl)-amino]-[2,4′]bipyridinyl-2′-yl}-amide;
(R)-Pyrrolidine-3-carboxylic acid {5′-chloro-6-[((S)-2,2-dimethyl-tetrahydro-pyran-4-ylmethyl)-amino]-[2,4′]bipyridinyl-2′-yl}-amide;
(R)-Pyrrolidine-3-carboxylic acid {5′-chloro-6-[((2R,6S)-2,6-dimethyl-tetrahydro-pyran-4-ylmethyl)-amino]-[2,4′]bipyridinyl-2′-yl}-amide;
(R)-Piperidine-3-carboxylic acid {5′-chloro-6-[((R)-2,2-dimethyl-tetrahydro-pyran-4-ylmethyl)-amino]-[2,4′]bipyridinyl-2′-yl}-amide;
(R)-Piperidine-3-carboxylic acid {5′-chloro-6-[((S)-6,6-dimethyl-[1,4]dioxan-2-ylmethyl)-amino]-[2,4′]bipyridinyl-2′-yl}-amide;
(R)-Piperidine-3-carboxylic acid {5′-chloro-6-[((R)-2,2-dimethyl-tetrahydro-pyran-4-ylmethyl)-amino]-5-fluoro-[2,4′]bipyridinyl-2′-yl}-amide;
(R)-Pyrrolidine-3-carboxylic acid {5′-chloro-6-[((R)-2,2-dimethyl-tetrahydro-pyran-4-ylmethyl)-amino]-[2,4′]bipyridinyl-2′-yl}-amide;
(R)-Piperidine-3-carboxylic acid {5′-chloro-6-[((S)-2,2-dimethyl-tetrahydro-pyran-4-ylmethyl)-amino]-[2,4′]bipyridinyl-2′-yl}-amide;
(R)-Piperidine-3-carboxylic acid {5′-chloro-6-[((S)-2,2-dimethyl-tetrahydro-pyran-4-ylmethyl)-amino]-5-fluoro-[2,4′]bipyridinyl-2′-yl}-amide;
(R)-Piperidine-3-carboxylic acid {5′-chloro-6-[((S)-6,6-dimethyl-[1,4]dioxan-2-ylmethyl)-amino]-5-fluoro-[2,4′]bipyridinyl-2′-yl}-amide;
(R)-Piperidine-3-carboxylic acid {5′-chloro-6-[((R)-6,6-dimethyl-[1,4]dioxan-2-ylmethyl)-amino]-[2,4′]bipyridinyl-2′-yl}-amide;
(R)-Piperidine-3-carboxylic acid {5′-chloro-6-[((R)-5,5-dimethyl-[1,4]dioxan-2-ylmethyl)-amino]-5-fluoro-[2,4′]bipyridinyl-2′-yl}-amide;
(R)-Piperidine-3-carboxylic acid {3,5′-dichloro-6-[(tetrahydro-pyran-4-ylmethyl)-amino]-[2,4′]bipyridinyl-2′-yl}-amide;
(R)-Piperidine-3-carboxylic acid {5′-chloro-5-fluoro-6-[(tetrahydro-pyran-4-ylmethyl)-amino]-[2,4′]bipyridinyl-2′-yl}-amide;
(R)-Piperidine-3-carboxylic acid {5′-chloro-3-fluoro-6-[(tetrahydro-pyran-4-ylmethyl)-amino]-[2,4′]bipyridinyl-2′-yl}-amide;
(R)-Piperidine-3-carboxylic acid {5′-chloro-6-[(tetrahydro-pyran-4-ylmethyl)-amino]-[2,4′]bipyridinyl-2′-yl}-amide;
(R)-Pyrrolidine-3-carboxylic acid {5′-chloro-6-[(tetrahydro-pyran-4-ylmethyl)-amino]-[2,4′]bipyridinyl-2′-yl}-amide;
(R)-Pyrrolidine-3-carboxylic acid {3,5′-dichloro-6-[(tetrahydro-pyran-4-ylmethyl)-amino]-[2,4′]bipyridinyl-2′-yl}-amide;
(R)-Pyrrolidine-3-carboxylic acid {5′-chloro-5-fluoro-6-[(tetrahydro-pyran-4-ylmethyl)-amino]-[2,4′]bipyridinyl-2′-yl}-amide;
(R)-Piperidine-3-carboxylic acid {5,5′-dichloro-6-[(tetrahydro-pyran-4-ylmethyl)-amino]-[2,4′]bipyridinyl-2′-yl}-amide;
(R)-Piperidine-3-carboxylic acid {3,5,5′-trichloro-6-[(tetrahydro-pyran-4-ylmethyl)-amino]-[2,4′]bipyridinyl-2′-yl}-amide;
(R)-Piperidine-3-carboxylic acid {3-chloro-5′-fluoro-6-[(tetrahydro-pyran-4-ylmethyl)-amino]-[2,4′]bipyridinyl-2′-yl}-amide;
(3R,6R)-6-Methyl-piperidine-3-carboxylic acid {5′-chloro-6-[((R)-2,2-dimethyl-tetrahydro-pyran-4-ylmethyl)-amino]-[2,4′]bipyridinyl-2′-yl}-amide;
(3R,5S)-5-Trifluoromethyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Ethyl-piperidine-3-carboxylic acid {5′-chloro-6-[(tetrahydro-pyran-4-ylmethyl)-amino]-[2,4′]bipyridinyl-2′-yl}-amide;
(3R,5S)-5-Methyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Methyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Methyl-piperidine-3-carboxylic acid {5′-chloro-6-[((S)-2,2-dimethyl-tetrahydro-pyran-4-ylmethyl)-amino]-[2,4′]bipyridinyl-2′-yl}-amide;
(3R,6R)-6-Methyl-piperidine-3-carboxylic acid {5′-chloro-5-fluoro-6-[(tetrahydro-pyran-4-ylmethyl)-amino]-[2,4′]bipyridinyl-2′-yl}-amide;
(3R,6S)-6-Methyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Ethyl-piperidine-3-carboxylic acid {5′-chloro-5-fluoro-6-[(tetrahydro-pyran-4-ylmethyl)-amino]-[2,4′]bipyridinyl-2′-yl}-amide;
(R)-Piperidine-3-carboxylic acid {5′-chloro-6-[(4-cyano-tetrahydro-pyran-4-ylmethyl)-amino]-[2,4′]bipyridinyl-2′-yl}-amide;
(R)-Piperidine-3-carboxylic acid {5′-chloro-6-[(4-methyl-tetrahydro-pyran-4-ylmethyl)-amino]-[2,4′]bipyridinyl-2′-yl}-amide;
(R)-Piperidine-3-carboxylic acid {5′-chloro-6-[(4-fluoro-tetrahydro-pyran-4-ylmethyl)-amino]-[2,4′]bipyridinyl-2′-yl}-amide;
(R)-Piperidine-3-carboxylic acid {5′-chloro-5-fluoro-6-[(4-methyl-tetrahydro-pyran-4-ylmethyl)-amino]-[2,4′]bipyridinyl-2′-yl}-amide;
(R)-Piperidine-3-carboxylic acid {3,5′-dichloro-6-[(4-methoxy-tetrahydro-pyran-4-ylmethyl)-amino]-[2,4′]bipyridinyl-2′-yl}-amide;
(R)-Piperidine-3-carboxylic acid {5′-chloro-5-fluoro-6-[(4-methoxy-tetrahydro-pyran-4-ylmethyl)-amino]-[2,4′]bipyridinyl-2′-yl}-amide;
(R)-Piperidine-3-carboxylic acid {5′-chloro-6-[(4-ethyl-tetrahydro-pyran-4-ylmethyl)-amino]-5-fluoro-[2,4′]bipyridinyl-2′-yl}-amide;
(1S,3R)-3-Amino-cyclopentanecarboxylic acid {5′-chloro-6-[(tetrahydro-pyran-4-ylmethyl)-amino]-[2,4′]bipyridinyl-2′-yl}-amide;
(R)-Piperidine-3-carboxylic acid [5′-chloro-6-(3-fluoro-benzylamino)-[2,4′]bipyridinyl-2′-yl]-amide;
6-Oxo-piperidine-3-carboxylic acid {5′-chloro-6-[(tetrahydro-pyran-4-ylmethyl)-amino]-[2,4′]bipyridinyl-2′-yl}-amide;
(1S,3R)-3-Amino-cyclopentanecarboxylic acid {3,5′-dichloro-6-[(tetrahydro-pyran-4-ylmethyl)-amino]-[2,4′]bipyridinyl-2′-yl}-amide;
(1R,3R)-3-Amino-cyclopentanecarboxylic acid {5′-chloro-6-[(tetrahydro-pyran-4-ylmethyl)-amino]-[2,4′]bipyridinyl-2′-yl}-amide;
(1R,3S)-3-Amino-cyclopentanecarboxylic acid {3,5′-dichloro-6-[(tetrahydro-pyran-4-ylmethyl)-amino]-[2,4′]bipyridinyl-2′-yl}-amide;
(R)-Piperidine-3-carboxylic acid [5′-chloro-6-(3,5-difluoro-benzylamino)-[2,4]bipyridinyl-2′-yl]-amide;
(1R,3S)-3-Amino-cyclopentanecarboxylic acid {5′-chloro-6-[(tetrahydro-pyran-4-ylmethyl)-amino]-[2,4′]bipyridinyl-2′-yl}-amide;
(3R,5S)-5-Methoxymethyl-pyrrolidine-3-carboxylic acid {5′-chloro-5-fluoro-6-[(4-methoxy-tetrahydro-pyran-4-ylmethyl)-amino]-[2,4′]bipyridinyl-2′-yl}-amide;
(3R,5S)-5-Methoxymethyl-pyrrolidine-3-carboxylic acid {5′-chloro-6-[(4-methyl-tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-6-[(2,2-dimethyl-tetrahydro-pyran-4-ylmethyl)-amino]-[2,4′]bipyridinyl-2′-yl}-amide;
(3R,5S)-5-Methoxymethyl-pyrrolidine-3-carboxylic acid {3,5′-dichloro-6-[(2,2-dimethyl-tetrahydro-pyran-4-ylmethyl)-amino]-[2,4′]bipyridinyl-2′-yl}-amide;
(3S,4R)-4-Methoxy-pyrrolidine-3-carboxylic acid {3,5′-dichloro-5-fluoro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-6-[(tetrahydro-pyran-4-ylmethyl)-amino]-[2,4′]bipyridinyl-2′-yl}-amide;
(3R,5S)-5-Methoxymethyl-pyrrolidine-3-carboxylic acid {3,5′-dichloro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {3,5′-dichloro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-5-fluoro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-6-[((2R,6S)-2,6-dimethyl-tetrahydro-pyran-4-ylmethyl)-amino]-[2,4′]bipyridinyl-2′-yl}-amide;
(R)-Morpholine-2-carboxylic acid {5′-chloro-5-fluoro-6-[(tetrahydro-pyran-4-ylmethyl)-amino]-[2,4′]bipyridinyl-2′-yl}-amide;
(S)-[1,4]-Oxazepane-6-carboxylic acid {3,5′-dichloro-6-[(tetrahydro-pyran-4-ylmethyl)-amino]-[2,4′]bipyridinyl-2′-yl}-amide;
(R)-Morpholine-2-carboxylic acid {5′-chloro-3-fluoro-6-[(tetrahydro-pyran-4-ylmethyl)-amino]-[2,4′]bipyridinyl-2′-yl}-amide;
(R)-Morpholine-2-carboxylic acid {3,5′-dichloro-6-[(tetrahydro-pyran-4-ylmethyl)-amino]-[2,4′]bipyridinyl-2′-yl}-amide;
(R)-Morpholine-2-carboxylic acid {5′-chloro-6-[((R)-2,2-dimethyl-tetrahydro-pyran-4-ylmethyl)-amino]-[2,4′]bipyridinyl-2′-yl}-amide;
(R)-Morpholine-2-carboxylic acid {3,5′-dichloro-6-[((R)-2,2-dimethyl-tetrahydro-pyran-4-ylmethyl)-amino]-[2,4′]bipyridinyl-2′-yl}-amide;
(R)-Morpholine-2-carboxylic acid {3,5′-dichloro-6-[((S)-2,2-dimethyl-tetrahydro-pyran-4-ylmethyl)-amino]-[2,4′]bipyridinyl-2′-yl}-amide;
(R)-Morpholine-2-carboxylic acid {5′-chloro-6-[(tetrahydro-pyran-4-ylmethyl)-amino]-[2,4′]bipyridinyl-2′-yl}-amide; and
(R)-Morpholine-2-carboxylic acid {5′-chloro-6-[((S)-2,2-dimethyl-tetrahydro-pyran-4-ylmethyl)-amino]-[2,4′]bipyridinyl-2′-yl}-amide.

Another embodiment of the present invention provides a compound of Formula II:

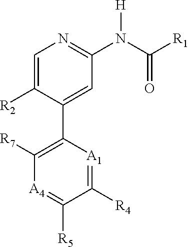

or a pharmaceutically acceptable salt thereof, wherein:

R₁ is selected from —(CH₂)₀₋₂-heteroaryl, —(CH₂)₀₋₂-aryl, C₁₋₈ alkyl, C₃₋₈ branched alkyl, C₃₋₈ cycloalkyl, and a 4 to 8 membered heterocycloalkyl group, wherein said groups are each independently optionally substituted;

R₂ is selected from hydrogen, C₁₋₄ alkoxy, C₁₋₄ haloalkyl, C₁₋₄-alkyl, and halogen;

A₁ is CR₃;

A₄ is N;

R₃ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, hydroxyl, CN, —O—C₁₋₄ alkyl, —O—C₁₋₄ haloalkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is selected from O, SO₂, and NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;

R₈ is selected from hydrogen, C₁₋₄ alkyl, and C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted; and

R¹⁴ is selected from hydrogen, phenyl, halogen, hydroxy, C₁₋₄-alkyl, H, C₃₋₆-branched alkyl, C₁₋₄-haloalkyl, CF₃, ═O, and O—C₁₋₄-alkyl.

A preferred embodiment provides a compound of Formula II, wherein:

R₁ is selected from —(CH₂)₀₋₂-heteroaryl, —(CH₂)₀₋₂-aryl, wherein said groups are each independently optionally substituted with one to three substituents selected from —NH₂, —F, —Cl, —OH, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ cyclo haloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;

R₂ is selected from hydrogen, C₁₋₄ alkoxy, C₁₋₄ haloalkyl, C₁₋₄-alkyl, and halogen;

A₁ is CR₃;

A₄ is N;

R₃ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;

R₄ is selected from hydrogen, halogen, 5 to 7 membered heterocyclyl-R¹⁴, or A₆-L-R₉;

R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is O, SO₂, or NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene;

R₈ is selected from hydrogen, C₁₋₄ alkyl, and C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;

R¹⁴ is selected from hydrogen, phenyl, halogen, hydroxy, C₁₋₄-alkyl, H, C₃₋₆-branched alkyl, C₁₋₄-haloalkyl, CF₃, ═O, and O—C₁₋₄-alkyl; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

Yet another preferred embodiment provides a compound of Formula II, wherein:

R₁ is selected from —(CH₂)₀₋₂-heteroaryl, and —(CH₂)₀₋₂-aryl, wherein said groups are each independently optionally substituted with one to three substituents selected from the group consisting of —NH₂, F, Cl, —OH, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, —O—(CH₂)₁₋₂—C₃₋₆ cycloalkyl-R¹⁴, —O—(CH₂)₁₋₂—C₄₋₆ heterocycloalkyl-R¹⁴, —SO₂—NR¹⁵R¹⁶, and —SO₂—C₃₋₅ cycloalkyl;

R₂ is selected from hydrogen, and halogen;

A₁ is CR₃;

A₄ is N;

R₃ is hydrogen;

R₄ is selected from piperidinyl, morpholinyl, pyrrolidinyl, and A₆-L-R₉; wherein each said piperidinyl, morpholinyl, pyrrolidinyl group is substituted with R¹⁴;

R₅ is selected from hydrogen, Cl, F, and CF₃;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and C₁₋₄ alkyl;

R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl;

R¹⁴ is selected from phenyl, halogen, hydroxyl, C₁₋₂-alkyl, CF₃, and hydrogen; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

Provided in yet another preferred embodiment is a compound of Formula II, wherein:

R₁ is selected from C₁₋₈ alkyl, C₃₋₈ cycloalkyl, C₃₋₈ branched alkyl, and a 4 to 8 membered heterocycloalkyl group, wherein said groups are each independently optionally substituted with one to three substituents selected from —NH₂, —F, —OH, ═O, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ halo-cycloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl; —C(O)—NR¹⁵R¹⁶, and —SO₂—NR¹⁵R¹⁶, and further wherein, any two said substituents along with the atoms to which they are attached can form a ring;

R₂ is selected from hydrogen, C₁₋₄ alkoxy, C₁₋₄ haloalkyl, C₁₋₄-alkyl, and halogen;

A₁ is CR₃;

A₄ is N;

R₃ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, and halogen;

R₄ is selected from hydrogen, halogen, 5 to 7 membered heterocyclyl-R¹⁴, and A₆-L-R₉;

R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;

R₇ is selected from hydrogen, C₁₋₄ alkyl, C₁₋₄ haloalkyl, O—C₁₋₃ alkyl, and halogen;

A₆ is selected from O, SO₂, and NR₈;

L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;

R₈ is selected from hydrogen, C₁₋₄ alkyl, and C₃₋₈ branched-alkyl, and —C₃₋₈ branched haloalkyl;

R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;

R¹⁴ is selected from hydrogen, phenyl, halogen, hydroxy, C₁₋₄-alkyl, H, C₃₋₆-branched alkyl, C₁₋₄-haloalkyl, CF₃, ═O, and O—C₁₋₄-alkyl; and

R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

A further preferred embodiment provides a compound of Formula II, wherein:

R₁ is selected from C₁₋₈ alkyl, C₃₋₈ branched alkyl, C₃₋₈ cycloalkyl, and a 4 to 8 membered heterocycloalkyl group, wherein said groups are each independently optionally substituted with one to three substituents selected from the group consisting of —NH₂, F, —OH, ═O, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;

R₂ is selected from hydrogen, and halogen;

A₁ is CR₃;

A₄ is N;

R₃ is hydrogen;

R₄ is selected from piperidinyl, morpholinyl, pyrrolidinyl, and A₆-L-R₉; wherein each said piperidinyl, morpholinyl, pyrrolidinyl group is substituted with R¹⁴;

R₅ is selected from hydrogen, Cl, F, and CF₃;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and C₁₋₄ alkyl;

R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl; and

R¹⁴ is selected from phenyl, halogen, hydroxy, C₁₋₂-alkyl, and hydrogen.

Provided in yet another preferred embodiment is a compound of Formula II, wherein:

R₁ is selected from piperidinyl, morpholinyl, 1-methylpiperidinyl, tetrahydro-pyran, pyrrolidinyl, tetrahydro-furan, azetidine, pyrrolidin-2-one, azepane, and 1,4-oxazepane, wherein said R₁ groups are each independently optionally substituted with one to three substituents selected from F, OH, NH₂, CO-methyl, —NH-methyl, ethyl, fluoro-ethyl, trifluoro-ethyl, (CH₂)₂-methoxy, SO₂—CH₃, COO—CH₃, SO₂-ethyl, SO₂-cyclopropyl, methyl, SO₂—CH—(CH₃)₂, NH—SO₂—CH₃, NH—SO₂—C₂H₅, ═O, CF₃, (CH₂)-methoxy, methoxy, NH—SO₂—CH—(CH₃)₂, —(CH₂)—O—(CH₂)₂-methoxy, —O—CH—(CH₃)₂;

R₂ is selected from Cl, and F;

A₁ is CR₃;

A₄ is N;

R₃ is hydrogen;

R₄ is A₆-L-R₉;

R₅ is selected from Cl, F, and hydrogen;

R₆ is H;

R₇ is selected from hydrogen, F, and Cl;

A₆ is NR₈;

L is selected from C₀₋₃-alkylene, —CD₂-, and C₃₋₈ branched alkylene;

R₈ is selected from hydrogen, and methyl; and

R₉ is selected from C₁₋₃ alkyl, C₄₋₆ branched alkyl, —(CH₂)₁₋₃—O—C₁₋₄ alkyl, —(CH₂)-pyridyl, benzyl, CD₂-tetrahydro-pyran, tetrahydro-pyran, tetrahydro-thiopyran 1,1-dioxide, piperidinyl, pyrrolidine-2-one, dioxane, cyclopropyl, tetrahydrofuran, cyclohexyl, and cycloheptyl, wherein said groups are optionally substituted with one to three substituents each independently selected from F, OCHF₂, CO-methyl, OH, methyl, methoxy, CN, ethyl, and NH—CO-methyl.

A particularly preferred embodiment of the present invention provides a compound of Formula II, wherein:

R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, —O—CH—(CH₃)₂;

R₂ is Cl;

A₁ is CR₃;

A₄ is N;

R₃ is hydrogen;

R₄ is A₆-L-R₉;

R₅ is selected from Cl, F, and hydrogen;

R₆ is H;

R₇ is selected from Cl, F, and hydrogen;

A₆ is NR₈;

L is selected from —CH₂—, —CD₂-;

R₈ is selected from hydrogen, and methyl; and

R₉ is selected from pyridyl, benzyl, tetrahydro-pyran, dioxane, tetrahydrofuran, wherein said groups are optionally substituted with one to three substituents each independently selected from F, OH, methyl, ethyl, methoxy, CN.

Particularly preferred compounds of Formula II are selected from:
(R)-Piperidine-3-carboxylic acid {2,5′-dichloro-5-[(tetrahydro-pyran-4-ylmethyl)-amino]-[3,4′]bipyridinyl-2′-yl}-amide;
(R)-Piperidine-3-carboxylic acid {6,5′-dichloro-5-[(tetrahydro-pyran-4-ylmethyl)-amino]-[3,4′]bipyridinyl-2′-yl}-amide; and
(R)-Piperidine-3-carboxylic acid {5′-chloro-5-[(tetrahydro-pyran-4-ylmethyl)-amino]-[3,4′]bipyridinyl-2′-yl}-amide.

Another embodiment provides a method of treating a disease or condition mediated by CDK9 by using a compound of Formula I or Formula II, or a pharmaceutically acceptable salt thereof. Also provided in another embodiment is the manufacture of a medicament for the treatment of a disease or condition mediated by CDK9, said medicament comprising a compound of Formula I or Formula II, or a pharmaceutically acceptable salt thereof.

Another aspect of the present invention provides a method of treating a disease or condition mediated by CDK9 using a compound of Formula I or Formula II, or pharmaceutically acceptable salt thereof. A preferred method comprises using a therapeutically effective amount of a compound of Formula I or Formula II.

The present invention also provides a pharmaceutical composition comprising a compound of Formula I or Formula II, or a pharmaceutically acceptable salt thereof, and a pharmaceutically acceptable carrier, diluent or excipient. Also provided in another embodiment is the use of a compound of Formula I or Formula II, or a pharmaceutically acceptable salt thereof in the manufacture of a medicament for the treatment of a disease or condition mediated by CDK9.

In another aspect, the present invention provides a method of regulating, modulating, or inhibiting protein kinase activity which comprises contacting a protein kinase with a compound of the invention. Suitable protein kinases include CDK1, CDK2, CDK3, CDK4, CDK5, CDK6, CDK7, CDK8 and CDK9, or any combination thereof. Preferably, the protein kinase is selected from the group consisting of CDK1, CDK2 and CDK9, or any combination thereof. In still another embodiment, the protein kinase is in a cell culture. In yet another embodiment, the protein kinase is in a mammal.

In another aspect, the invention provides a method of treating a protein kinase-associated disorder comprising administering to a subject in need thereof a pharmaceutically acceptable amount of a compound of the invention. Suitable protein kinases include CDK1, CDK2, CDK3, CDK4, CDK5, CDK6, CDK7, CDK8 and CDK9 or combinations thereof (preferably, the protein kinase is selected from the group consisting of CDK1, CDK2 and CDK9, more preferably, the protein kinase is CDK9.) Suitable CDK combinations include CDK4 and CDK9; CDK1, CDK2 and CDK9; CDK9 and CDK7; CDK9 and CDK1; CDK9 and CDK2; CDK4, CDK6 and CDK9; CDK1, CDK2, CDK3, CDK4, CDK6 and CDK9.

In yet another aspect, the invention provides a method of treating cancer comprising administering to a subject in need thereof a pharmaceutically acceptable amount of a compound of the invention. Suitable cancers for treatment include bladder, head and neck, breast, stomach, ovary, colon, lung, brain, larynx, lymphatic system, hematopoetic system, genitourinary tract, gastrointestinal, ovarian, prostate, gastric, bone, small-cell lung, glioma, colorectal and pancreatic cancer.

### DEFINITIONS

As used herein, the term “protein kinase-associated disorder” includes disorders and states (e.g., a disease state) that are associated with the activity of a protein kinase, e.g., the CDKs, e.g., CDK1, CDK2 and/or CDK9. Non-limiting examples of protein kinase-associated disorders include abnormal cell proliferation (including protein kinase-associated cancers), viral infections, fungal infections, autoimmune diseases and neurodegenerative disorders.

The term “treat,” “treated,” “treating” or “treatment” includes the diminishment or alleviation of at least one symptom associated or caused by the state, disorder or disease being treated. In certain embodiments, the treatment comprises the induction of a protein kinase-associated disorder, followed by the activation of the compound of the invention, which would in turn diminish or alleviate at least one symptom associated or caused by the protein kinase-associated disorder being treated. For example, treatment can be diminishment of one or several symptoms of a disorder or complete eradication of a disorder.

The term “use” includes one or more of the following embodiments of the invention, respectively: the use in the treatment of protein kinase-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from cancer, inflammation, cardiac hypertrophy, and HIV infection, as well as those diseases that depend on the activity of protein kinases. The term “use” further includes embodiments of compositions herein which bind to a protein kinase sufficiently to serve as tracers or labels, so that when coupled to a fluor or tag, or made radioactive, can be used as a research reagent or as a diagnostic or an imaging agent.

The term “alkyl,” by itself or as part of another substituent, means, unless otherwise stated, a fully saturated straight-chain (linear; unbranched) or branched chain, having the number of carbon atoms specified, if designated (i.e. C₁-C₁₀ means one to ten carbons). Illustrative “alkyl” group examples are methyl, ethyl, n-propyl, isopropyl, n-butyl, t-butyl, isobutyl, sec-butyl, n-pentyl, n-hexyl, n-heptyl, n-octyl, and the like. If no size is designated, the alkyl groups mentioned herein contain 1-10 carbon atoms, typically 1-8 carbon atoms, and preferably 1-6 or 1-4 carbon atoms.

The terms “alkoxy,” refers to —O-alkyl, wherein the term alkyl is as defined above.

The term “cycloalkyl” by itself or in combination with other terms, represents, unless otherwise stated, cyclic versions of alkyl. Additionally, cycloalkyl may contain fused rings, but excludes fused aryl and heteroaryl groups. Cycloalkyl groups, unless indicated otherwise, are unsubstituted. Illustrative examples of cycloalkyl are cyclopropyl, cyclobutyl, cyclopentyl, cyclohexyl, cycloheptyl, norbornyl, and the like. If no ring size is specified, the cycloalkyl groups described herein generally contain 3-10 ring members, preferably 3-6 ring members.

The term “heterocyclic” or “heterocycloaklyl” or “heterocyclyl,” by itself or in combination with other terms, represents a cycloalkyl containing at least one annular carbon atom and at least one annular heteroatom selected from the group consisting of O, N, P, Si and S, preferably from N, O and S, wherein the ring is not aromatic but can contain unsaturations. The nitrogen and sulfur atoms in a heterocyclic group may optionally be oxidized and the nitrogen heteroatom may optionally be quaternized. The heterocyclic groups discussed herein, if not otherwise specified, contain 3-10 ring members, and at least one ring member is a heteroatom selected from N, O, P, Si, and S. Preferably, not more than three of these heteroatoms are included in a heterocyclic group, and generally not more than two of these heteroatoms are present in a single ring of the heterocyclic group. The heterocyclic group can be fused to an additional carbocylic or heterocyclic ring. A heterocyclic group can be attached to the remainder of the molecule at an annular carbon or annular heteroatom. Additionally, heterocyclic may contain fused rings, but excludes fused systems containing a heteroaryl group as part of the fused ring system. Illustrative examples of heterocyclic groups include, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, piperidin-2-one, azepane, tetrahydro-2H-pyranyl, pyrrolidinyl, methylpyrrolidinone, alkylpiperidinyl, haloalkylperidinyl, 1-(alkylpiperidin-1-yl)ethanone, and the like.

The term “aryl”, unless otherwise stated, represents an aromatic hydrocarbon group which can be a single ring or multiple rings (e.g., from 1 to 3 rings) which are fused together. Aryl includes fused rings, wherein one or more of the fused rings is fully saturated (e.g., cycloalkyl) or partially unsaturated (e.g., cyclohexenyl), but not a heterocyclic or heteroaromatic ring. Illustrative examples of aryl groups include, but are not limited to, phenyl, 1-naphthyl, 2-naphthyl, and tetrahydronaphthyl.

The term “heteroaryl”, as used herein, refers to groups comprising a single ring, or a fused ring, where at least one of the rings is an aromatic ring that contain from one to four heteroatoms selected from N, O, and S as ring members (i.e., it contains at least one heteroaromatic ring), wherein the nitrogen and sulfur atoms can be oxidized, and the nitrogen atom(s) can be quaternized. A heteroaryl group can be attached to the remainder of the molecule through an annular carbon or annular heteroatom, and it can be attached through any ring of the heteroaryl moiety, if that moiety is a bicyclic, tricyclic, or a fused ring system. A heteroaryl group may contain fused rings, wherein one of the fused rings is aromatic or hetero aromatic, and the other fused ring(s) are partially unsaturated (e.g., cyclohexenyl, 2,3-dihydrofuran, tetrahydropyrazine, and 3,4-dihydro-2H-pyran), or completely saturated (e.g., cyclohexyl, cyclopentyl, tetrahydrofuran, morpholine, and piperazine). The term heteroaryl is also intended to include fused rings systems that include a combination of aromatic and heteroaromatic rings systems (e.g., indoles, quinoline, quinazolines, and benzimidazoles). Illustrative examples of heteroaryl groups are 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

The terms “halo” or “halogen,” represents a fluorine, chlorine, bromine, or iodine atom. The term “haloalkyl,” represents an alkyl group as defined above, wherein one or more hydrogen atoms of the alkyl group are replaced by a halogen atom which may be the same or different. The term haloalkyl thus includes mono-haloalkyl, di-haloalkyl, tri-haloalkyl, tetra-haloalkyl, and the like as well as per-haloalkyl. The prefix “perhalo” refers to the respective group wherein all available valences are replaced by halo groups. For example “perhaloalkyl” includes —CCl₃, —CF₃, —CCl₂CF₃, and the like. The terms “perfluoroalkyl” and “perchloroalkyl” are a subset of perhaloalkyl wherein all available valences are replaced by fluoro and chloro groups, respectively. Illustrative examples of perfluoroalkyl include —CF₃ and —CF₂CF₃, and of perchloroalkyl include —CCl₃ and —CCl₂CCl₃.

“Optionally substituted” as used herein indicates that the particular group or groups being described may have no non-hydrogen substituents (i.e., it can be unsubstituted), or the group or groups may have one or more non-hydrogen substituents. If not otherwise specified, the total number of such substituents that may be present is equal to the number of H atoms present on the unsubstituted form of the group being described. Typically, an optionally substituted group will contain up to four (1-4) substituents. Where an optional substituent is attached via a double bond, such as a carbonyl oxygen (═O), the group takes up two available valences on the group being substituted, so the total number of substituents that may be included is reduced according to the number of available valences. Suitable optional substituent groups include halo, C₁₋₄alkyl, —NH—C(O)—CH₂—O—C₁₋₄ alkyl, —NHC(O)—C₁₋₄ alkyl, —C(O)—O—C₁₋₄alkyl, —O—C₁₋₄alkyl, —O—C₁₋₄haloalkyl, —C₁₋₄alkylene-O—C₁₋₄haloalkyl, —C₁₋₄alkylene-O—C₁₋₄alkyl, —NH—C₁₋₄alkyl, —C(O)—CH₂—O—C₁₋₄ alkyl, —C(O)—O—C₃₋₆ branched alkyl, —C₁₋₄ haloalkyl, —(CH₂)₁₋₃—O—C₁₋₂ alkyl, —C₁₋₄-cycloalkyl, —C₁₋₄alkylene-O—C₁₋₄alkyl, —NH₂, —SO₂—C₁₋₄alkyl, —NH—C(O)—C₁₋₄ alkyl, and —NH—SO₂—C₁₋₄ alkyl, hydroxyl, nitro, cyano, oxo, —C(O)—C₁₋₄alkyl, —C(O)— and the like.

“Unless specified otherwise, the term “compounds of the present invention” refer to compounds of Formula I, prodrugs thereof, pharmaceutically acceptable salts of the compounds, and/or prodrugs, and hydrates or solvates of the compounds, salts, and/or prodrugs, as well as, all stereoisomers (including diastereoisomers and enantiomers), tautomers, and isotopically labeled compounds (including deuterium substitutions), as well as inherently formed moieties (e.g., polymorphs, solvates and/or hydrates).

As used herein, the term “pharmaceutically acceptable salts” refers to salts that retain the biological effectiveness and properties of the compounds of this invention and, which typically are not biologically or otherwise undesirable.

The term “a therapeutically effective amount” of a compound of the present invention refers to an amount of the compound of the present invention that when administered to a subject, is effective to (1) at least partially alleviating, inhibiting, preventing and/or ameliorating a condition, or a disorder or a disease (i) mediated by one or more CDK enzymes, or (ii) associated with one or more CDK enzyme activities, or (iii) characterized by activity of proteins regulated (directly or indirectly) by one or more CDK enzymes (e.g. RNA polymerase II); or (2) reducing or inhibiting the expression of proteins whose expression is dependent (directly or indirectly) on one or more CDK enzymes (e.g. Mcl-1, Cyclin D, Myc etc.). When used in conjunction with a cell, the term “a therapeutically effective amount” refers to the amount of the compound of the present invention that, when administered to a cell, or a tissue, or a non-cellular biological material, or a medium, is effective to at least partially reducing or inhibiting the activity of proteins regulated by one or more CDK enzymes; or at least partially reducing or inhibiting the expression of proteins whose expression is dependent (directly or indirectly) on one or more CDK enzymes.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

Unless defined otherwise or clearly indicated by context, all technical and scientific terms used herein have the same meaning as commonly understood by one of ordinary skill in the art to which this invention belongs.

### DETAILED DESCRIPTION

The compounds disclosed herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine experimentation.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

The various starting materials, intermediates, and compounds of the embodiments may be isolated and purified, where appropriate, using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention (e.g., formulas I or II), will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

Compounds of the embodiments may generally be prepared using a number of methods familiar to one skilled in the art.

The compounds of the present invention can be isolated and used per se or as their pharmaceutical acceptable salt. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The compounds of the present invention also include isotopically labelled forms of the compounds which may be synthesized using the processes described herein or modifications thereof known by those of skill in the art. Isotopically labelled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labelled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labelled compound may be particularly desirable for PET or SPECT studies. Isotopically labelled compounds of this invention and prodrugs thereof can generally be prepared by carrying out the procedures disclosed in the schemes or in the examples and preparations described below by substituting a readily available isotopically labelled reagent for a non-isotopically labelled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

Compounds of the present invention include isomers including all stereoisomers of the compounds referred to in the formulas herein, including enantiomers, diastereomers, as well as all conformers, rotamers, and tautomers, unless otherwise indicated. The invention includes all enantiomers of any chiral compound disclosed, in either substantially pure levorotatory or dextrorotatory form, or in a racemic mixture, or in any ratio of enantiomers.

Furthermore, the compounds disclosed herein may contain one or more chiral centers. Accordingly, if desired, such compounds can be prepared or isolated as pure stereoisomers, i.e., as individual enantiomers or diastereomers, or as stereoisomer enriched mixtures. All such stereoisomers (and enriched mixtures) are included within the scope of the embodiments, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Unless stereochemistry is explicitly indicated in a chemical structure or chemical name, the chemical structure or chemical name is intended to embrace all possible stereoisomers, conformers, rotamers, and tautomers of the compound depicted. For example, a compound containing a chiral carbon atom is intended to embrace both the (R) enantiomer and the (S) enantiomer, as well as mixtures of enantiomers, including racemic mixtures; and a compound containing two chiral carbons is intended to embrace all enantiomers and diastereomers (including (R,R), (S,S), (R,S), and (R,S) isomers).

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. As defined herein, solvates and hydrates of the compounds of the present invention are considered compositions, wherein the composition comprises a compound of the present invention and a solvent (including water).

The compounds of the present invention may exist in either amorphous or polymorphic form; therefore, all physical forms are considered to be within the scope of the present invention.

Compounds of the invention, i.e. compounds of the present invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

In certain uses of the compounds of the present invention, it may be advantageous to use a pro-drug of the compound. In general, pro-drugs convert in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Typically, the compounds of the present invention are administered as a pharmaceutical composition. A typical pharmaceutical composition comprises a compound of the present invention and a pharmaceutically acceptable carrier, diluent or excipient. As used herein, the term “pharmaceutically acceptable carriers, diluents or excipients” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical composition can be formulated for particular routes of administration such as oral administration, and parenteral administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminium silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.
   Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

The invention further provides pharmaceutical compositions and dosage forms that may comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of Formula I in free form or in pharmaceutically acceptable salt form, exhibit valuable pharmacological properties, e.g. CDK inhibiting properties, e.g. as indicated in in vitro and in vivo tests as provided below and are therefore indicated for therapy.

When used with respect to methods of treatment/prevention and the use of the compounds and formulations thereof described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.). Typically, when a step of administering a compound of the invention is disclosed herein, the invention further contemplates a step of identifying an individual or subject in need of the particular treatment to be administered or having the particular condition to be treated.

### EXAMPLES

Referring to the examples that follow, compounds of the embodiments were synthesized using the methods described herein, or other methods known to one skilled in the art. The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18 5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on LCMS instruments: Waters System (Acuity UPLC and a Micromass ZQ mass spectrometer; Column: Acuity HSS C18 1.8-micron, 2.1×50 mm; gradient: 5-95% acetonitrile in water with 0.05% TFA over a 1.8 min period; flow rate 1.2 mL/min; molecular weight range 200-1500; cone Voltage 20 V; column temperature 50° C.). All masses were reported as those of the protonated parent ions.

### Specific Optical Rotation

The specific optical rotation was measured on an Autopol IV Automatic Polarimeter (Rudolph Research Analytical) with a 100-mm path-length cylindrical glass cell at 20° C. temperature. The wavelength of the light used was 589 nanometer (the sodium D line). Optical rotation of the same cell filled with solvent was subtracted as blank. The final result was the average of two measurements, each over 10 seconds. The 10 mg/mL sample solution was prepared using MeOH as solvent.

GCMS analysis is performed on a Hewlett Packard instrument (HP6890 Series gas chromatograph with a Mass Selective Detector 5973; injector volume: 1 ┌L; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.) or Varian 400 MHz MR NMR (Palo Alto, Calif.). The spectral reference was either TMS or the known chemical shift of the solvent. Some compound samples were run at elevated temperatures (e.g., 75° C.) to promote increased sample solubility. Melting points are determined on a Laboratory Devices MeI-Temp apparatus (Holliston, Mass.).

Preparative separations are carried out using a Combiflash Rf system (Teledyne Isco, Lincoln, Nebr.) with RediSep silica gel cartridges (Teledyne Isco, Lincoln, Nebr.) or SiliaSep silica gel cartridges (Silicycle Inc., Quebec City, Canada) or by flash column chromatography using silica gel (230-400 mesh) packing material, or by HPLC using a Waters 2767 Sample Manager, C-18 reversed phase column, 30×50 mm, flow 75 mL/min. Typical solvents employed for the Combiflash Rf system and flash column chromatography are dichloromethane, methanol, ethyl acetate, hexane, heptane, acetone, aqueous ammonia (or ammonium hydroxide), and triethyl amine. Typical solvents employed for the reverse phase HPLC are varying concentrations of acetonitrile and water with 0.1% trifluoroacetic acid.

The following abbreviations have the following meanings. If not specifically defined, abbreviations will have their generally accepted meanings

### Abbreviations

### ACN: Acetonitrile

BINAP: 2,2′-bis(diphenylphosphino)-1,1′-binapthyl
BOC-anhydride: di-tert-butyl dicarbonate
bp: boiling point
d: days
DAST: Diethylaminosulfur trifluoride
DBU: 1,8-Diazabicyclo[5.4.0]undec-7-ene

### DCM: Dichloromethane

DIEA: diisopropylethylamine

### DIPEA: N,N-diisopropylethylamine

### DMAP: 4-Dimethylaminopyridine

DME: 1,2-dimethoxyethane

### DMF: N,N-dimethylformamide

DMSO: dimethyl sulfoxide
dppf: 1,1′-bis(diphenylphosphino)ferrocene
eq: equivalent
EtOAc: ethyl acetate
EtOH: ethanol
GCMS: gas chromatography-mass spectrometry
HATU: 2-(7-aza-1H-benzotriazole-1-yl)-1,1,3,3-tetramethyluronium hexafluorophosphate
HPLC or hplc: high performance liquid chromatography
hr: hour
hrs: hours
KO-tBu: potassium tert-butoxide
LHMDS: Lithium bis(trimethylsilyl)amide
MCPBA: meta-chloroperoxybenzoic acid
MeOH: methanol
n.a.: not available
NaH: sodium hydride

### NBS: N-bromosuccinimide

NEt₃: triethylamine
NMP: N-methyl-2-pyrrolidone
Rt: retention time
THF: tetrahydrofuran
TLC: thin layer chromatography

### Synthetic Procedures

Compounds of the present invention can be synthesized by procedures known to one skilled in the art, and the general schemes outlined below.

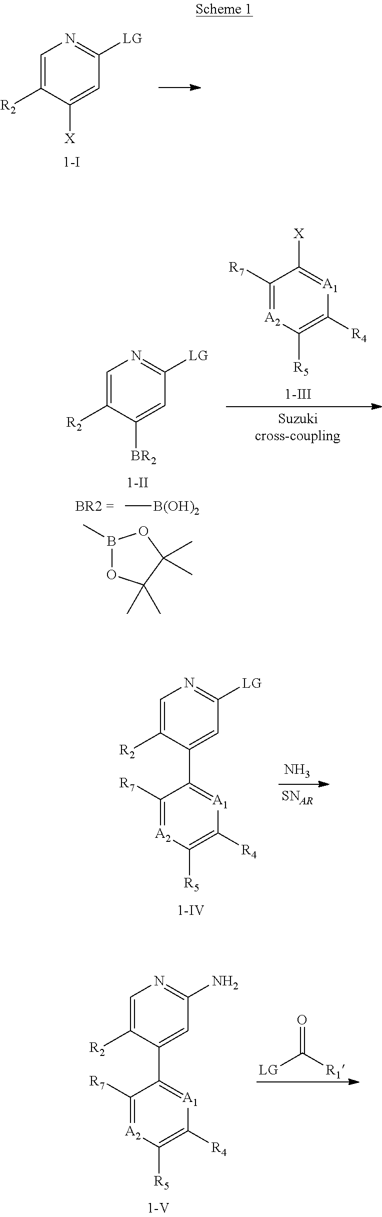

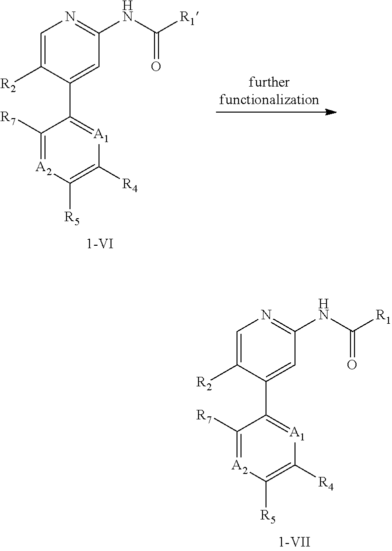

As shown in Scheme 1, synthesis can start with a functionalized pyridine I wherein LG is a leaving group such as F, Cl, OTf, and the like. X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and pyridine III then gives bi-heteroaryl intermediate IV. The SN_{AR} reaction between IV and ammonium hydroxide in a solvent such as DMF, THF, DMSO, NMP, dioxane with heating (30-130° C.) can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional manipulation is needed to obtain VII. When R₁′ is identical to R₁, compound VII will be the same as compound VI.

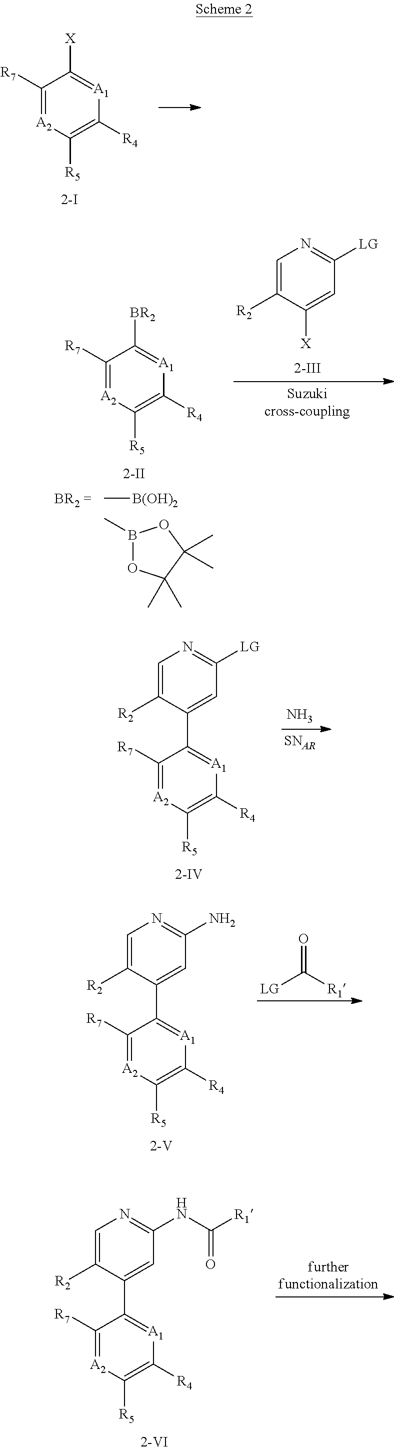

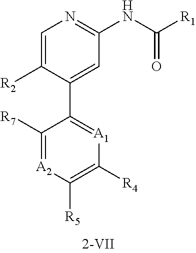

Another alternative route is illustrated in Scheme 2. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. The SN_{AR} reaction between IV and ammonium hydroxide in a solvent such as DMF, THF, DMSO, NMP, dioxane with heating (30-130° C.) can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional manipulation is needed to obtain VII. When R₁′ is identical to R₁, compound VII will be the same as compound VI.

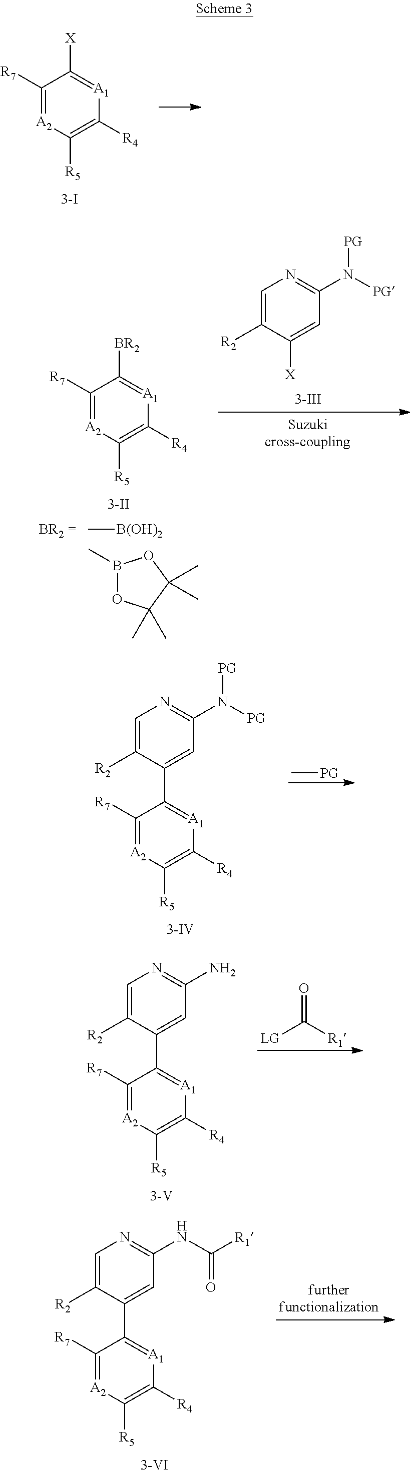

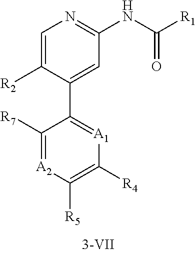

Another alternative route is illustrated in Scheme 3. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. Removal of protecting groups PG can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional manipulation is needed to obtain VII. When R₁′ is identical to R₁, compound VII will be the same as compound VI.

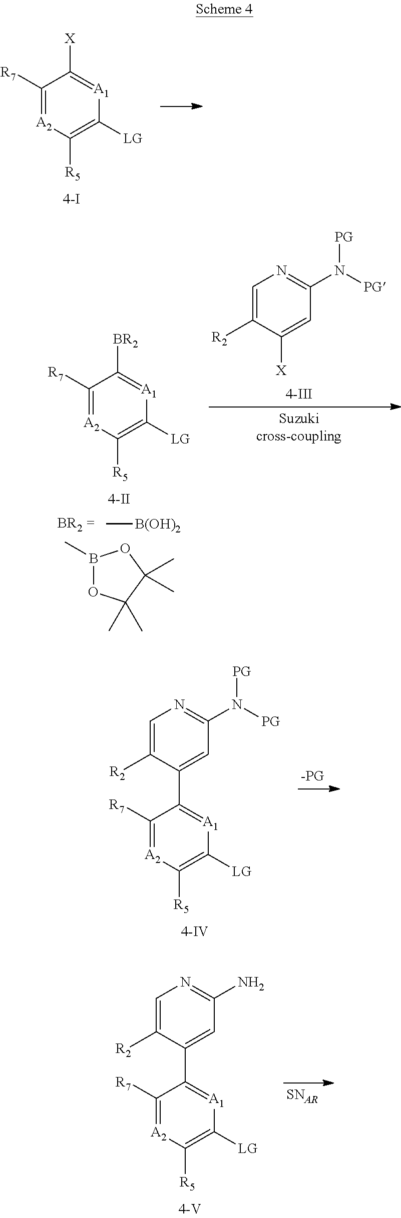

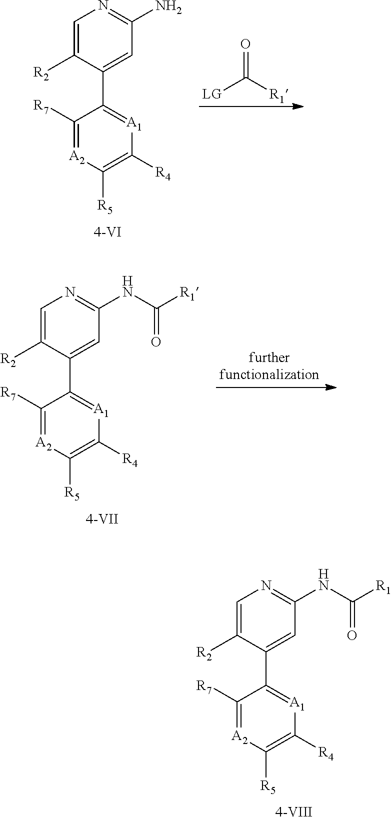

Another alternative route is illustrated in Scheme 4. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. Removal of protecting groups PG can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional manipulation is needed to obtain VIII. When R₁′ is identical to R₁, compound VIII will be the same as compound VII.

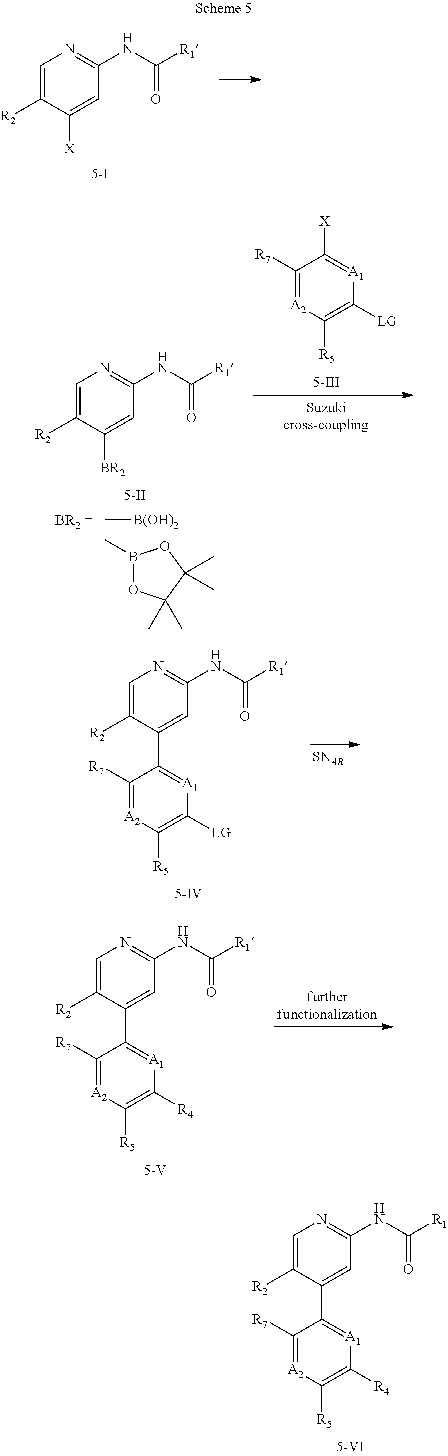

Another alternative route is illustrated in Scheme 5. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound V. When R₁′ is not identical to R₁, further functional manipulation is needed to obtain VI. When R₁′ is identical to R₁, compound VI will be the same as compound V.

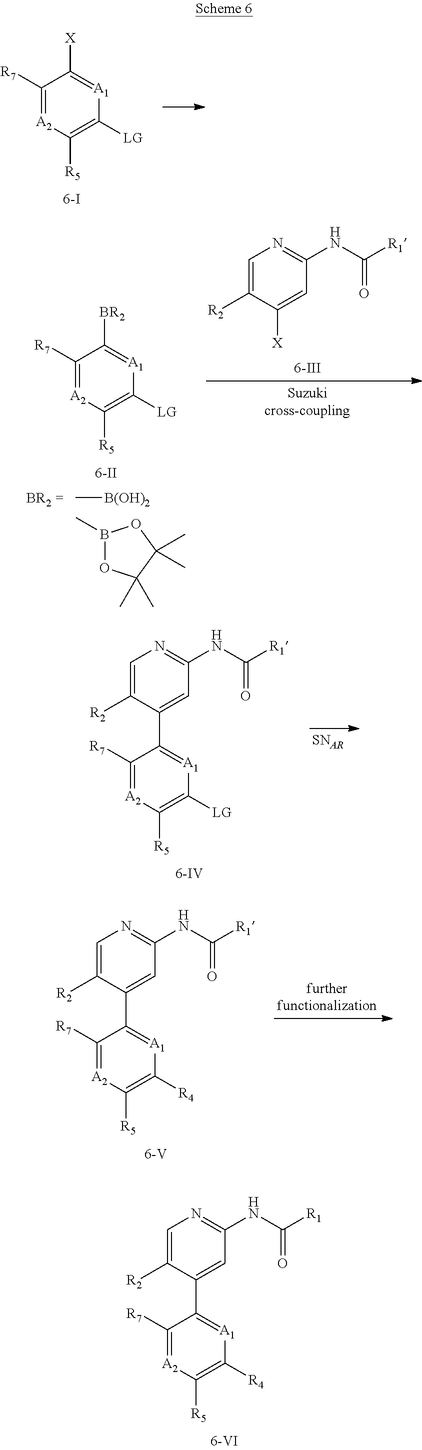

Another alternative route is illustrated in Scheme 6. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound V. When R₁′ is not identical to R₁, further functional manipulation is needed to obtain VI. When R₁′ is identical to R₁, compound VI will be the same as compound V.

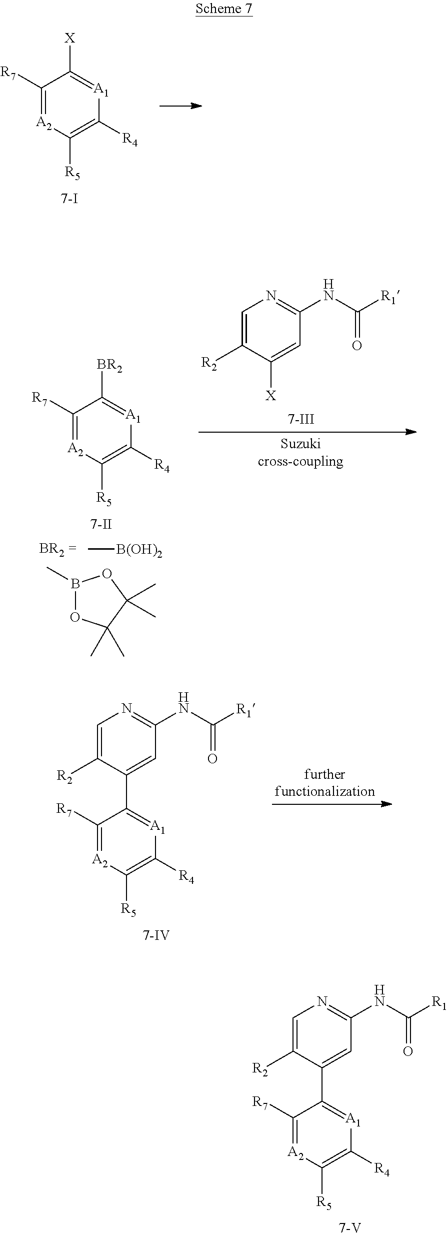

Another alternative route is illustrated in Scheme 7. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. When R₁′ is not identical to R₁, further functional manipulation is needed to obtain VI. When R₁′ is identical to R₁, compound VI will be the same as compound V.

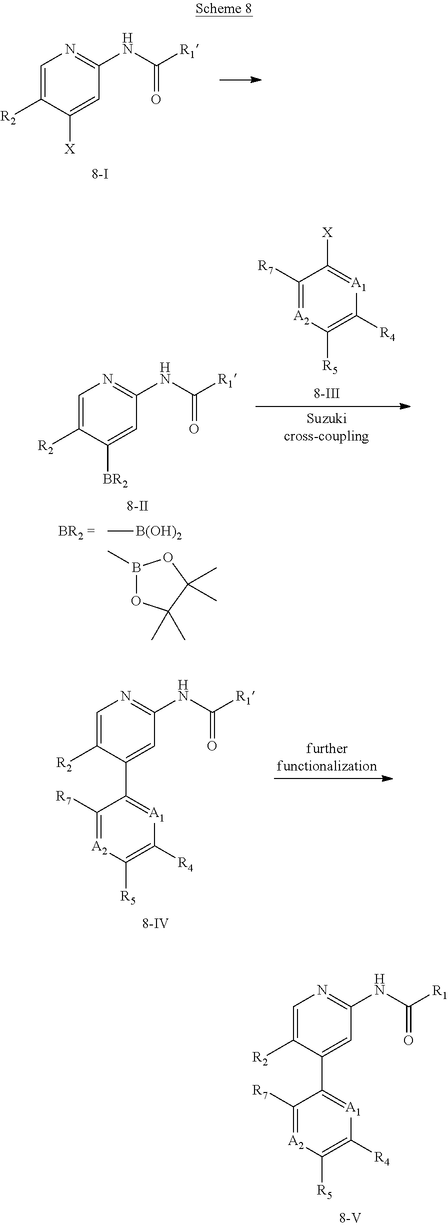

Another alternative route is illustrated in Scheme 8. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. When R₁′ is not identical to R₁, further functional manipulation is needed to obtain VI. When R₁′ is identical to R₁, compound VI will be the same as compound V.

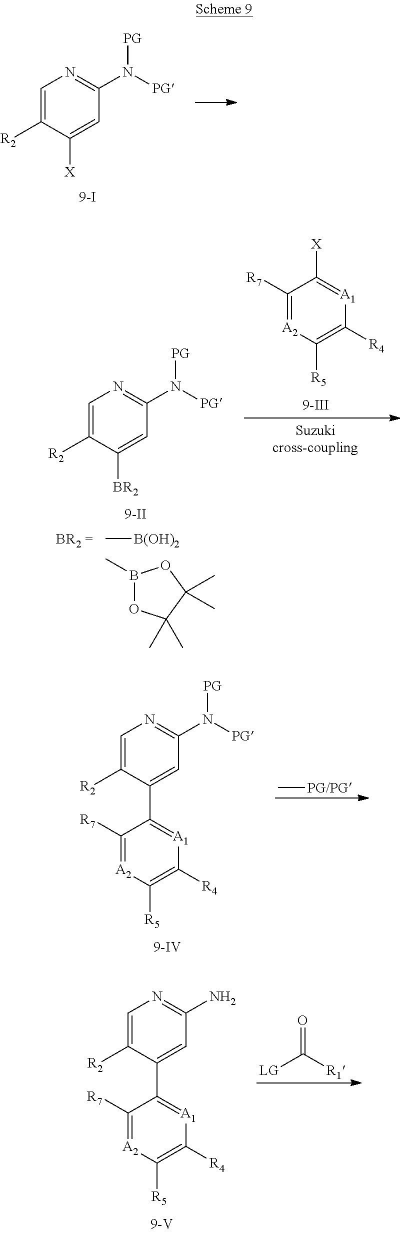

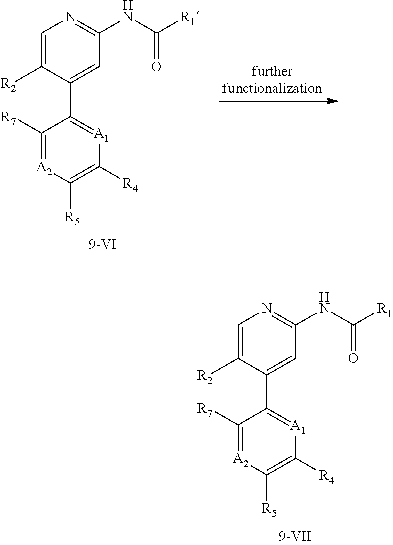

Another alternative route is illustrated in Scheme 9. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. Removal of protecting groups PG can give compound V. Coupling of the nascent amino pyridine V with an acyl intermediate bearing a leaving group in the presence of a base such as Et₃N, iPr₂NEt or pyridine in a solvent such as DMF, THF, DMSO, NMP, dioxane can give compound VI. When R₁′ is not identical to R₁, further functional manipulation is needed to obtain VII. When R₁′ is identical to R₁, compound VII will be the same as compound VI.

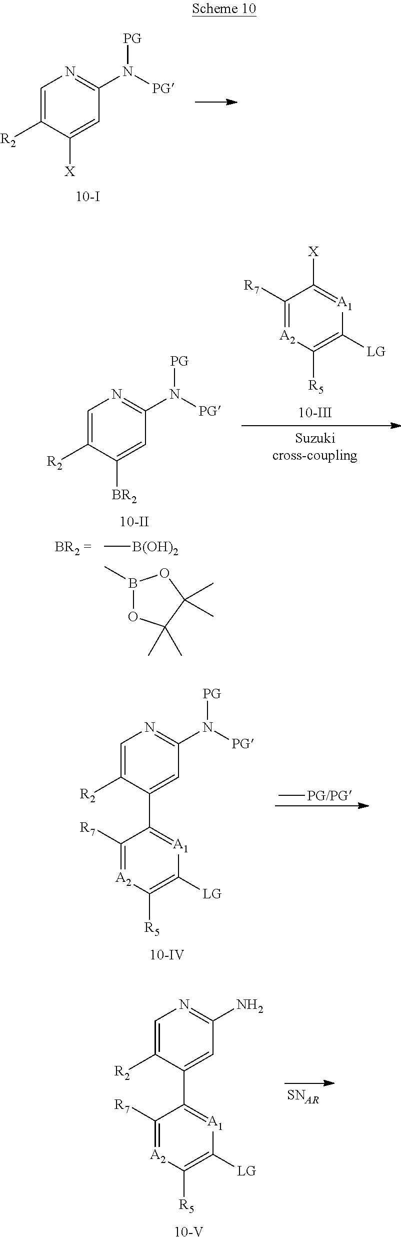

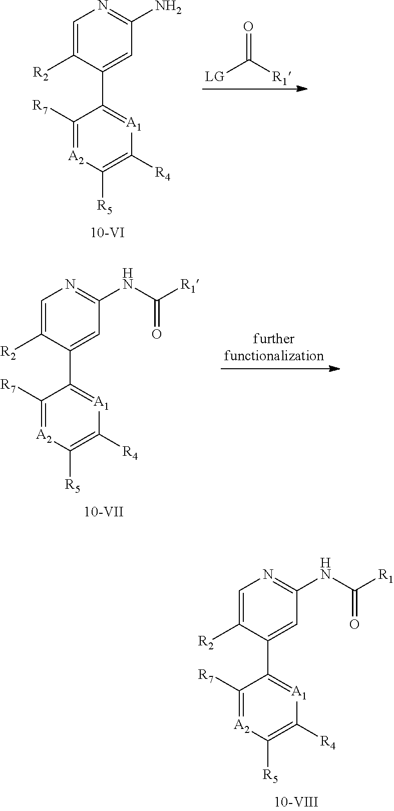

Another alternative route is illustrated in Scheme 10. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. Removal of protecting groups PG can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional manipulation is needed to obtain VIII. When R₁′ is identical to R₁, compound VIII will be the same as compound VII.

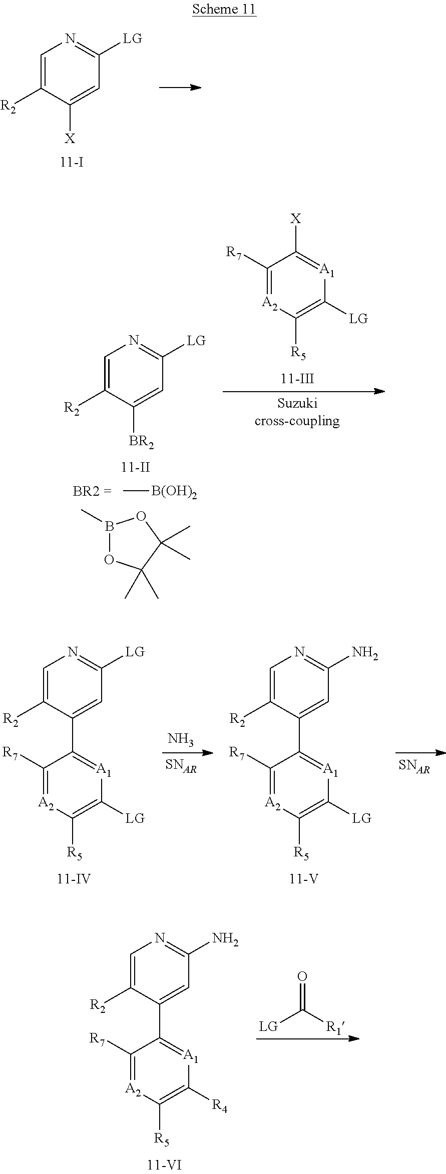

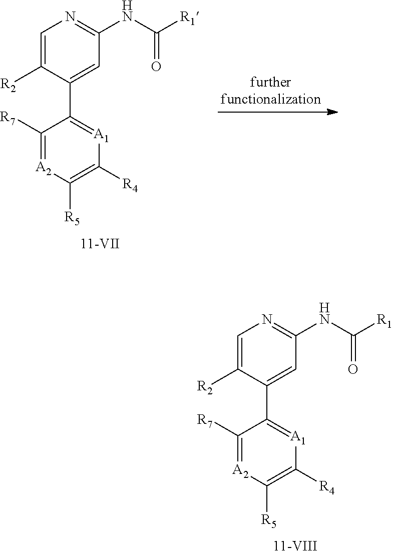

Another alternative route is illustrated in Scheme 11. Synthesis can start with a functionalized pyridine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. The SN_{AR} reaction between IV and ammonium hydroxide in a solvent such as DMF, THF, DMSO, NMP, dioxane with heating (30-130° C.) can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional manipulation is needed to obtain VIII. When R₁′ is identical to R₁, compound VIII will be the same as compound VII.

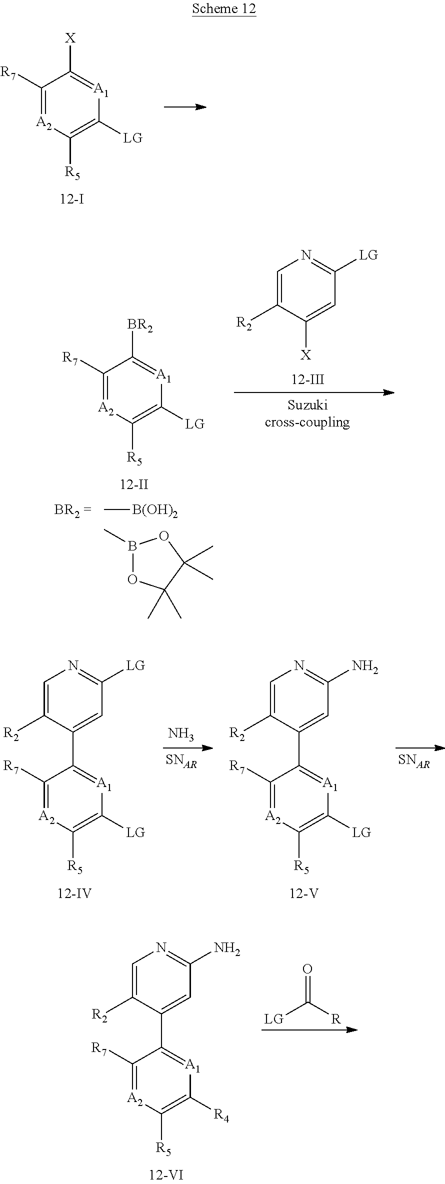

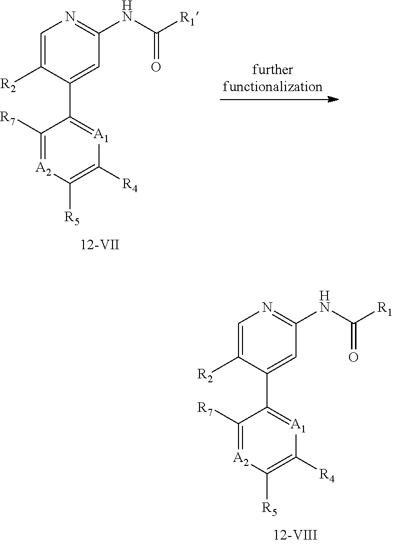

Another alternative route is illustrated in Scheme 12. Synthesis can start with a functionalized pyridine or pyrazine I wherein X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained.

Suzuki cross-coupling reaction between compound II and functionalize pyridine III then gives bi-heteroaryl intermediate IV. The SN_{AR} reaction between IV and ammonium hydroxide in a solvent such as DMF, THF, DMSO, NMP, dioxane with heating (30-130° C.) can give compound V. The SN_{AR} reaction between V and a functionalized amine NH₂R₁′ under basic condition (DIEA, TEA, lutidine, pyridine) in a solvent such as DMF, THF, DMSO, NMP, dioxane with heating (30-180° C.) can give compound VI. Coupling of the nascent amino pyridine VI with an acyl intermediate bearing a leaving group in the presence of a base such as Et₃N, iPr₂NEt or pyridine in a solvent such as DMF, THF, DMSO, NMP, dioxane can give compound VII. When R₁′ is not identical to R₁, further functional manipulation is needed to obtain VIII. When R₁′ is identical to R₁, compound VIII will be the same as compound VII.

### Synthesis of Intermediates

### Synthesis of 6-bromo-N-(3-fluorobenzyl)pyridin-2-amine

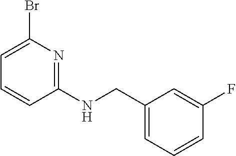

To a solution of 2,6-dibromopyridine (7.1 g, 30.0 mmol) in NMP (16 mL) was added (3-fluorophenyl)methanamine (4.13 g, 33.0 mmol) and Huenig's Base (5.76 mL, 33.0 mmol). The mixture was stirred under argon at 115-120° C. for 168 hrs. The mixture was cooled to room temperature and diluted with EtOAc (250 mL). The separated organic layer was washed with saturated aqueous sodium bicarbonate (2×), water (2×), brine (1×), dried over sodium sulphate, filtered off, and concentrated under reduced pressure. The crude material was purified by column chromatography [silica gel, 120 g, EtOAc/hexane=0/100 to 20/80] providing 6-bromo-N-(3-fluorobenzyl)pyridin-2-amine (7.11 g). LCMS (m/z): 281.1/283.1 [M+H]+; Rt=1.03 min.

### Synthesis of 5′-chloro-N6-(3-fluorobenzyl)-2,4′-bipyridine-2′,6-diamine

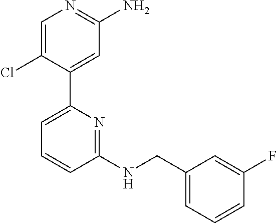

### Step 1: Preparation of 5′-chloro-2′-fluoro-N-(3-fluorobenzyl)-2,4′-bipyridin-6-amine

To 6-bromo-N-(3-fluorobenzyl)pyridin-2-amine (2.0 g, 7.11 mmol) were added 5-chloro-2-fluoropyridin-4-ylboronic acid (2.0 g, 11.4 mmol), PdCl₂(dppf) CH₂Cl₂ adduct (0.465 g, 0.569 mmol), DME (27 mL) and 2M aqueous sodium carbonate solution (9.25 mL, 18.50 mmol). The mixture was stirred at 100° C. for 3 hrs. After cooling to room temperature the mixture was diluted with EtOAc (25 mL) and MeOH (20 mL), filtered off and concentrated under reduced pressure. The crude material was purified by column chromatography [silica gel, 120 g, EtOAc/hexane=0/100 to 20/80] providing 5′-chloro-2′-fluoro-N-(3-fluorobenzyl)-2,4′-bipyridin-6-amine (1.26 g). LCMS (m/z): 332.2 [M+H]+; Rt=0.92 min.

### Step 2: Preparation of 5′-chloro-N6-(3-fluorobenzyl)-2,4′-bipyridine-2′,6-diamine

A mixture of 5′-chloro-2′-fluoro-N-(3-fluorobenzyl)-2,4′-bipyridin-6-amine (50 mg, 0.151 mmol) and ammonium hydroxide (aqueous solution 30-35 wt. %, 1 mL) in DMSO (1.3 mL) in a sealed microwave tube and under argon was heated in a microwave at 115° C. for 200 min. The mixture was diluted with EtOAc (50 mL) and water. The separated organic layer was washed with water (1×), brine (1×), dried over sodium sulfate, filtered off and concentrated under reduced pressure providing crude 5′-chloro-N6-(3-fluorobenzyl)-2,4′-bipyridine-2′,6-diamine (40 mg), which was directly used in the next step without further purification. LCMS (m/z): 329.0 [M+H]+; Rt=0.61 min.

### Alternative Preparation of 5′-chloro-N6-(3-fluorobenzyl)-2,4′-bipyridine-2′,6-diamine

A mixture of 5′-chloro-2′-fluoro-N-(3-fluorobenzyl)-2,4′-bipyridin-6-amine (0.2165 g, 0.653 mmol) and ammonium hydroxide (aqueous solution 30-35 wt. %, 3 mL) in DMSO (3 mL) was heated in a steel bomb at 120° C. for 21 hrs. The reaction mixture was diluted with water (25 mL) and extracted with EtOAc (3×25 mL). The combined extracts were washed with water (3×50 mL) and brine (1×50 mL), dried over sodium sulphate, filtered off and concentrated under reduced pressure. The residue was purified by column chromatography [silica gel, 40 g, EtOAc/heptane=50/50 to 100/0]. Pure fractions were combined and concentrated under reduced pressure providing 5′-chloro-N6-(3-fluorobenzyl)-2,4′-bipyridine-2′,6-diamine (0.1194 g). LCMS (m/z): 329.0 [M+H]+; Rt=0.68 min.

### Synthesis of 6-bromo-N-((tetrahydro-2H-pyran-4-yl)methyl)pyridin-2-amine

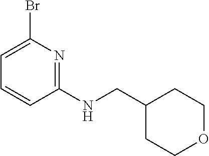

To a solution of 2-bromo-6-fluoropyridine (750 mg, 4.26 mmol) in DMSO (3 mL) was added (tetrahydro-2H-pyran-4-yl)methanamine hydrochloride (775 mg, 5.11 mmol) and triethylamine (1.426 mL, 10.23 mmol). The mixture was heated at 110° C. for 18 hrs. The mixture was allowed to cool to room temperature and diluted with EtOAc. The organic layer was washed with saturated aqueous sodium bicarbonate solution, water, and brine and dried over sodium sulfate, filtered off and concentrated under reduced pressure. The residue was purified by column chromatography [silica gel, 40 g, EtOAc/heptane=0/100 to 30/70]. Pure fractions were combined and concentrated under reduced pressure providing 6-bromo-N-((tetrahydro-2H-pyran-4-yl)methyl)pyridin-2-amine (940 mg) as a white solid. LCMS (m/z): 271.0/272.9 [M+H]+; Rt=0.81 min.

### Synthesis of 5′-chloro-N6-((tetrahydro-2H-pyran-4-yl)methyl)-2,4′-bipyridine-2′,6-diamine

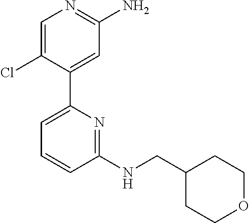

### Method A

### Step 1: Preparation of 5′-chloro-2′-fluoro-N-((tetrahydro-2H-pyran-4-yl)methyl)-2,4′-bipyridin-6-amine

A mixture of 6-bromo-N-((tetrahydro-2H-pyran-4-yl)methyl)pyridin-2-amine (271 mg, 1 mmol), 5-chloro-2-fluoropyridin-4-ylboronic acid (351 mg, 2.000 mmol), PdCl₂(dppf) CH₂Cl₂ adduct (82 mg, 0.100 mmol) in DME (4.5 mL) and 2M aqueous sodium carbonate solution (318 mg, 3.00 mmol) was heated in a sealed tube at 103° C. for 2 hrs. The mixture was cooled to room temperature and was diluted with EtOAc (˜25 mL) and MeOH (˜5 mL), filtered and concentrated under reduced pressure. The residue was purified by column chromatography [silica gel, 12 g, EtOAc/heptane=10/90 to 50/50]. Fractions were combined and concentrated under reduced pressure providing 5′-chloro-2′-fluoro-N-((tetrahydro-2H-pyran-4-yl)methyl)-2,4′-bipyridin-6-amine (260 mg). LCMS (m/z): 322.1/323.9 [M+H]+; Rt=0.60 min.

### Step 2: Preparation of 5′-chloro-N6-((tetrahydro-2H-pyran-4-yl)methyl)-2,4′-bipyridine-2′,6-diamine

### Method A-2-1

A mixture of 5′-chloro-2′-fluoro-N-((tetrahydro-2H-pyran-4-yl)methyl)-2,4′-bipyridin-6-amine (150 mg, 0.466 mmol) and ammonium hydroxide (aqueous solution 30-35 wt. %, 1.5 mL) in DMSO (1.8 mL) was placed under argon in a sealed microwave tube, and then heated in a microwave at 125° C. for 210 min. The mixture was cooled to ambient temperature, and diluted with EtOAc and brine. The separated organic layer was separated, and washed with water, brine, dried over sodium sulfate, filtered off and concentrated under reduced pressure providing crude 5′-chloro-N6-((tetrahydro-2H-pyran-4-yl)methyl)-2,4′-bipyridine-2′,6-diamine (140 mg), which was directly used in the next step without further purification. LCMS (m/z): 318.9/320.8 [M+H]+; Rt=0.44 min.

### Method A-2-2

A mixture of 5′-chloro-2′-fluoro-N-((tetrahydro-2H-pyran-4-yl)methyl)-2,4′-bipyridin-6-amine (6 g, 18.65 mmol) and ammonium hydroxide (aqueous solution 30-35 wt. %, 60 mL) in DMSO (35 mL) was heated in a steel bomb at 140° C. for 4 days. The mixture was allowed to cool to room temperature, diluted with water (500 mL) and vigorously stirred for ˜3.5 hrs. The resulting fine solid was filtered off, and rinsed with water (˜100 mL). The solid was suspended in MeOH (30 mL), warmed up to reflux for ˜5 min and afterwards sonicated for 5 min at room temperature. The suspension was allowed to cool to room temperature, and water (60 mL) was added slowly. The suspension was stirred vigorously for ˜5 min, filtered off and rinsed with water (˜100 mL). The solid was dried in high vacuo for 16 hrs providing crude 5′-chloro-N6-((tetrahydro-2H-pyran-4-yl)methyl)-2,4′-bipyridine-2′,6-diamine (5.52 g) light brownish solid, which was directly used in the next step without further purification. LCMS (m/z): 319.1 [M+H]+; Rt=0.43 min.

### Method B

### Step 1: Preparation of {5′-chloro-6-[(tetrahydro-pyran-4-ylmethyl)-amino]-[2,4′]bipyridinyl-2′-yl}-carbamic acid tert-butyl ester

A mixture of 6-bromo-N-((tetrahydro-2H-pyran-4-yl)methyl)pyridin-2-amine (15.5 g, 57.2 mmol) and 2-(tert-butoxycarbonylamino)-5-chloropyridin-4-ylboronic acid (17.13 g, 62.9 mmol) in DME (293 mL), PdCl₂(dppf) CH₂Cl₂ adduct (4.67 g, 5.72 mmol), and 2M aqueous sodium carbonate solution (97.5 mL, 195 mmol) was stirred at 98° C. for 22 hrs under argon. The reaction mixture was diluted with EtOAc and stirred for additional 30 min. The organic layer was separated and washed with saturated aqueous sodium bicarbonate solution, water, and brine. The organic phase was dried over sodumsulfate, filtered off and concentrated under reduced pressure. The residue was purified by column chromatography [silica gel, EtOAc/heptane=5/95 to 60/40] providing {5′-chloro-6-[(tetrahydro-pyran-4-ylmethyl)-amino]-[2,4′]bipyridinyl-2′-yl}-carbamic acid tert-butyl ester as solid (6.72 g). LCMS (m/z): 419.2 [M+H]+; Rt=0.74 min.

### Step 2: Preparation of 5′-chloro-N6-((tetrahydro-2H-pyran-4-yl)methyl)-2,4′-bipyridine-2′,6-diamine

To a solution of {5′-chloro-6-[(tetrahydro-pyran-4-ylmethyl)-amino]-[2,4′]bipyridinyl-2′-yl}-carbamic acid tert-butyl ester (6.8 g, 16.23 mmol) in MeOH (7 mL) was added 4N hydrochloride in dioxane (110 mL, 440 mmol), and the resulting reaction mixture was stirred at 25° C. for 4.5 hrs. The mixture was concentrated under reduced pressure and the residue was diluted with EtOAc. The organic layer was separated, washed with saturated aqueous sodium bicarbonate solution and brine, dried over sodium sulphate, filtered off and concentrated under reduced pressure providing 5′-chloro-N6-((tetrahydro-2H-pyran-4-yl)methyl)-2,4′-bipyridine-2′,6-diamine as solid (5.77 g), which was directly used in the next step without further purification. LCMS (m/z): 319.1 [M+H]+; Rt=0.43 min.

### Synthesis of 5-bromo-N-((tetrahydro-2H-pyran-4-yl)methyl)pyridin-3-amine

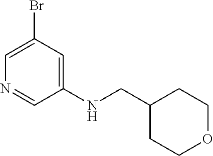

A mixture of Pd(OAc)₂ (95 mg, 0.422 mmol) and BINAP (315 mg, 0.507 mmol) in dioxane (8 mL) in a sealed tube was stirred for ˜5 min. 3,5-Dibromopyridine (1000 mg, 4.22 mmol) and (tetrahydro-2H-pyran-4-yl)methanamine hydrochloride (640 mg, 4.22 mmol) were added and stirring was continued for additional ˜5 min. KOtBu (521 mg, 4.64 mmol) was added and the mixture was heated at 93° C. for ˜18 hrs. The mixture was cooled to room temperature, diluted with EtOAc (˜50 mL) and MeOH (˜10 mL), filtered off and concentrated under reduced pressure. The residue was purified by column chromatography [silica gel, 40 g, EtOAc/heptane=30/70 to 90/10]. Fractions were combined and concentrated under reduced pressure providing 5-bromo-N-((tetrahydro-2H-pyran-4-yl)methyl)pyridin-3-amine (146 mg). LCMS (m/z): 270.9/272.9 [M+H]+; Rt=0.46 min.

### Synthesis of 5′-chloro-N5-((tetrahydro-2H-pyran-4-yl)methyl)-3,4′-bipyridine-2′,5-diamine

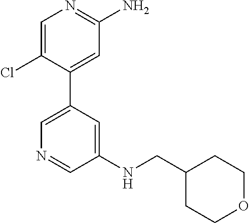

### Step 1: Preparation of 5′-chloro-2′-fluoro-N-((tetrahydro-2H-pyran-4-yl)methyl)-3,4′-bipyridin-5-amine

A mixture of 5-bromo-N-((tetrahydro-2H-pyran-4-yl)methyl)pyridin-3-amine (146 mg, 0.538 mmol), 5-chloro-2-fluoropyridin-4-ylboronic acid (189 mg, 1.077 mmol), PdCl₂(dppf) CH₂Cl₂ adduct (44.0 mg, 0.054 mmol) in DME (2.7 mL) and 2M aqueous sodium carbonate solution (0.9 mL, 1.800 mmol) in a sealed tube was heated at 103° C. for 2 hrs. The mixture then was cooled to room temperature, diluted with EtOAc (˜25 mL) and MeOH (˜5 mL), filtered and concentrated under reduced pressure. The residue was purified by column chromatography [silica gel, 12 g, EtOAc/heptane=50/50 to 90/10]. Fractions were combined and concentrated under reduced pressure providing 5′-chloro-2′-fluoro-N-((tetrahydro-2H-pyran-4-yl)methyl)-3,4′-bipyridin-5-amine (109 mg). LCMS (m/z): 322.0/323.9 [M+H]+; Rt=0.56 min.

### Step 2: Preparation of 5′-chloro-N-5-((tetrahydro-2H-pyran-4-yl)methyl)-3,4′-bipyridine-2′,5-diamine

A mixture of 5′-chloro-2′-fluoro-N-((tetrahydro-2H-pyran-4-yl)methyl)-3,4′-bipyridin-5-amine (110 mg, 0.342 mmol) and ammonium hydroxide (aqueous solution 30-35 wt. %, 1.5 mL) in DMSO (1.8 mL) was placed in a sealed microwave tube, under argon, and heated at 125° C. for 210 min. The heated mixture was cooled and diluted with EtOAc and brine. The organic layer was separated, and washed with water, brine, dried over sodium sulfate, filtered off and concentrated under reduced pressure providing crude 5′-chloro-N5-((tetrahydro-2H-pyran-4-yl)methyl)-3,4′-bipyridine-2′,5-diamine (82 mg), which was directly used in the next step without further purification. LCMS (m/z): 318.9/320.7 [M+H]+; Rt=0.38 min.

### Synthesis of 5-bromo-2-chloro-N-((tetrahydro-2H-pyran-4-yl)methyl)pyridin-3-amine

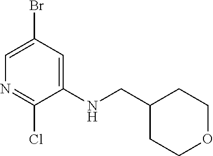

A solution of 5-bromo-2-chloropyridin-3-amine (1.3 g, 6.27 mmol) in DMF (20 mL) was added slowly sodium hydride (60 wt. % in mineral oil, 0.301 g) was stirred for 20 min, followed by addition of (tetrahydro-2H-pyran-4-yl)methyl 4-methylbenzenesulfonate (1.694 g, 6.27 mmol). The resulting reaction mixture was stirred at room temperature for 58 hrs, diluted with EtOAc, washed with water, brine, dried over sodium sulphate, filtered off and concentrated under reduced pressure. The residue was purified by column chromatography (silica gel, EtOAc/hexane=22/78) providing 5-bromo-2-chloro-N-((tetrahydro-2H-pyran-4-yl)methyl)pyridin-3-amine (1.27 g). LCMS (m/z): 305.0 [M+H]+; Rt=0.89 min.

### Synthesis of 5′,6-dichloro-N-5-((tetrahydro-2H-pyran-4-yl)methyl)-3,4′-bipyridine-2′,5-diamine

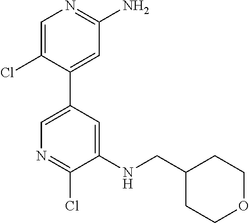

### Step 1: Preparation of 5′,6-dichloro-2′-fluoro-N-((tetrahydro-2H-pyran-4-yl)methyl)-3,4′-bipyridin-5-amine

To a suspension of 5-bromo-2-chloro-N-((tetrahydro-2H-pyran-4-yl)methyl)pyridin-3-amine (1 g, 3.27 mmol), 2M aqueous sodium carbonate solution (4.25 mL, 8.51 mmol), and 5-chloro-2-fluoropyridin-4-ylboronic acid (0.975 g, 5.56 mmol) in DME (20 mL) was added PdCl₂(dppf) CH₂Cl₂ adduct (0.214 g, 0.262 mmol). The reaction mixture then was heated in a sealed tube at 100° C. for 4 hrs. The reaction mixture was cooled, and diluted with EtOAc, the organic layer was separated, and washed with water and brine, dried over sodium sulphate, filtered off and concentrated under reduced pressure. The residue was purified by column chromatography (silica gel, EtOAc/hexane=1/3) providing 5′,6-dichloro-2′-fluoro-N-((tetrahydro-2H-pyran-4-yl)methyl)-3,4′-bipyridin-5-amine (693 mg). LCMS (m/z): 356.0 [M+H]+; Rt=0.96 min.

### Step 2: Preparation of 5′,6-dichloro-N-5-((tetrahydro-2H-pyran-4-yl)methyl)-3,4′-bipyridine-2′,5-diamine

A mixture of 5′,6-dichloro-2′-fluoro-N-((tetrahydro-2H-pyran-4-yl)methyl)-3,4′-bipyridin-5-amine (55 mg, 0.154 mmol) and ammonium hydroxide (aqueous solution 30-35 wt. %, 1.5 mL) in DMSO (1.8 mL) in a sealed microwave tube and under argon was heated in a microwave at 125° C. for 210 min. The mixture was diluted with EtOAc and brine. The separated organic layer was washed with water, brine, dried over sodium sulfate, filtered off and concentrated under reduced pressure providing crude 5′,6-dichloro-N-5-((tetrahydro-2H-pyran-4-yl)methyl)-3,4′-bipyridine-2′,5-diamine (55 mg), which was directly used in the next step without further purification. LCMS (m/z): 352.9/354.8 [M+H]+; Rt=0.60 min.

### Synthesis of 6-bromo-5-chloro-N-((tetrahydro-2H-pyran-4-yl)methyl)pyridin-2-amine (A) and 6-bromo-3-chloro-N-((tetrahydro-2H-pyran-4-yl)methyl)pyridin-2-amine (B)

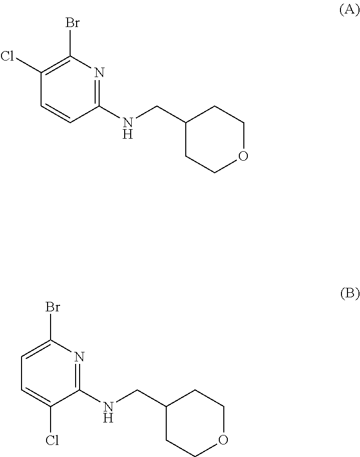

To a solution of 6-bromo-N-((tetrahydro-2H-pyran-4-yl)methyl)pyridin-2-amine (1000 mg, 3.69 mmol) in chloroform (15 mL) was added 1-chloropyrrolidine-2,5-dione (N-chlorosuccinimide, 492 mg, 3.69 mmol), and the resulting mixture was heated in a sealed tube at 33° C. for 16 hrs. The temperature was raised to 37° C. and heating was continued for 24 hrs. The temperature was raised to 43° C. and heating was continued for 5 days. The mixture was cooled to room temperature and diluted with 1N aqueous sodium hydroxide solution and dichloromethane. The separated organic layer was washed with brine, dried over sodium sulfate, filtered off and concentrated under reduced pressure. The residue was purified by column chromatography [silica gel, 80 g, EtOAc/heptane=5/95 to 35/65]. Fractions were combined and concentrated under reduced pressure yielding 6-bromo-3-chloro-N-((tetrahydro-2H-pyran-4-yl)methyl)pyridin-2-amine (B, 453 mg) and 6-bromo-5-chloro-N-((tetrahydro-2H-pyran-4-yl)methyl)pyridin-2-amine (A, ˜500 mg)

(B): LCMS (m/z): 305.0 [M+H]+; Rt=1.01 min. ¹³C NMR (150 MHz, DMSO-d6) δ [ppm]: 154.1, 138.5, 137.0, 114.5, 113.0, 66.7, 46.4, 39.8, 39.7, 39.5, 39.4, 39.3, 39.1, 34.2, 30.5.

(A): LCMS (m/z): 305.0 [M+H]+; Rt=0.96 min.

### Synthesis of 3,5′-dichloro-N6-((tetrahydro-2H-pyran-4-yl)methyl)-2,4′-bipyridine-2′,6-diamine

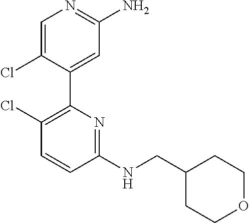

### Step 1: Preparation of 3,5′-dichloro-2′-fluoro-N-((tetrahydro-2H-pyran-4-yl)methyl)-2,4′-bipyridin-6-amine

A mixture of 6-bromo-5-chloro-N-((tetrahydro-2H-pyran-4-yl)methyl)pyridin-2-amine (300 mg, 0.982 mmol), 5-chloro-2-fluoropyridin-4-ylboronic acid (344 mg, 1.963 mmol), PdCl₂(dppf) CH₂Cl₂ adduct (80 mg, 0.098 mmol) in DME (4.5 mL) and 2M aqueous sodium carbonate (4.5 mL, 4.50 mmol) in a sealed tube was heated at 103° C. for 16 hrs. The mixture was allowed to cool to room temperature and was diluted with EtOAc (˜100 mL) and saturated aqueous sodium carbonate solution. The separated organic layer was washed with saturated aqueous sodium carbonate solution (2×), dried over sodium sulfate, filtered off and concentrated under reduced pressure. The residue was purified by column chromatography [silica gel, 25 g, EtOAc/heptane=0/100 to 25/75]. Fractions were combined and concentrated under reduced pressure providing 3,5′-dichloro-2′-fluoro-N-((tetrahydro-2H-pyran-4-yl)methyl)-2,4′-bipyridin-6-amine (140 mg). LCMS (m/z): 356.1 [M+H]+; Rt=0.96 min.

### Step 2: Preparation of 3,5′-dichloro-N6-((tetrahydro-2H-pyran-4-yl)methyl)-2,4′-bipyridine-2′,6-diamine

A mixture of 3,5′-dichloro-2′-fluoro-N-((tetrahydro-2H-pyran-4-yl)methyl)-2,4′-bipyridin-6-amine and ammonium hydroxide (aqueous solution 30-35 wt. %) in DMSO was heated in a steel bomb at 135° C. for 16 hrs. The mixture was cooled to room temperature and diluted with EtOAc. The separated organic layer was washed with water, saturated aqueous bicarbonate solution and brine and dried over sodium sulfate, filtered off and concentrated under reduced pressure. The crude material of 3,5′-dichloro-N6-((tetrahydro-2H-pyran-4-yl)methyl)-2,4′-bipyridine-2′,6-diamine (135 mg) was directly used in the next reaction without further purification. LCMS (m/z): 352.9 [M+H]+; Rt=0.67 min.

### Synthesis of 5,5′-dichloro-N6-((tetrahydro-2H-pyran-4-yl)methyl)-2,4′-bipyridine-2′,6-diamine

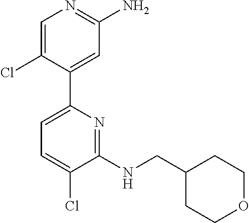

### Step 1: Preparation of 5,5′-dichloro-2′-fluoro-N-((tetrahydro-2H-pyran-4-yl)methyl)-2,4′-bipyridin-6-amine

A mixture of 6-bromo-3-chloro-N-((tetrahydro-2H-pyran-4-yl)methyl)pyridin-2-amine (200 mg, 0.654 mmol), 5-chloro-2-fluoropyridin-4-ylboronic acid (230 mg, 1.309 mmol), PdCl₂(dppf) CH₂Cl₂ adduct (53.4 mg, 0.065 mmol) in DME (3 mL) and 2M aqueous sodium carbonate (3 mL, 6.00 mmol) in a sealed tube was heated at 103° C. for 16 hrs. The mixture was cooled to ambient temperature and was diluted with EtOAc (˜100 mL) and saturated aqueous sodium bicarbonate solution. The separated organic layer was washed with saturated aqueous sodium bicarbonate solution (2×), dried over sodium sulfate, filtered off and concentrated under reduced pressure. The residue was purified by column chromatography [silica gel, 25 g, EtOAc/heptane=0/100 to 30/70]. Fractions were combined and concentrated under reduced pressure providing 5,5′-dichloro-2′-fluoro-N-((tetrahydro-2H-pyran-4-yl)methyl)-2,4′-bipyridin-6-amine (130 mg). LCMS (m/z): 356.1 [M+H]+; Rt=1.10 min.

### Step 2: Preparation of 5,5′-dichloro-N6-((tetrahydro-2H-pyran-4-yl)methyl)-2,4′-bipyridine-2′,6-diamine

A mixture of 5,5′-dichloro-2′-fluoro-N-((tetrahydro-2H-pyran-4-yl)methyl)-2,4′-bipyridin-6-amine, from step 1 above, and ammonium hydroxide (aqueous solution 30-35 wt. %) in DMSO was heated in a steel bomb at 135° C. for 16 hrs. The mixture was allowed to cool to room temperature and diluted with EtOAc. The separated organic layer was washed with water, saturated aqueous sodium bicarbonate solution and brine and dried over sodium sulfate, filtered off and concentrated under reduced pressure. The crude material of 5,5′-dichloro-N6-((tetrahydro-2H-pyran-4-yl)methyl)-2,4′-bipyridine-2′,6-diamine (116 mg) was directly used in the next reaction without further purification. LCMS (m/z): 352.9 [M+H]+; Rt=0.74 min.

### Synthesis of 6-bromo-3,5-dichloro-N-((tetrahydro-2H-pyran-4-yl)methyl)pyridin-2-amine

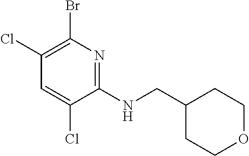

### Step 1: Preparation of 6-bromo-3,5-dichloro-N-((tetrahydro-2H-pyran-4-yl)methyl)-pyridin-2-amine/6-bromo-3-chloro-N-((tetrahydro-2H-pyran-4-yl)methyl)-pyridin-2-amine

A solution of 6-bromo-N-((tetrahydro-2H-pyran-4-yl)methyl)pyridin-2-amine (20 g, 74 mmol) in acetonitrile (240 mL) and N-chlorosuccinimide (9.85 g, 74 mmol) was heated to 80° C. for 3 hrs. The reaction mixture was allowed to cool to room temperature and concentrated under reduced pressure. The residue was diluted with brine (200 mL) and extracted with EtOAc (3×200 mL). The combined organic layers were concentrated under reduced pressure. The residue was purified by column chromatography [silica gel, EtOAc/heptane=0/100 to 50/50] providing 6-bromo-5-chloro-N-((tetrahydro-2H-pyran-4-yl)methyl)pyridin-2-amine (12 g) and a mixture of 6-bromo-3,5-dichloro-N-((tetrahydro-2H-pyran-4-yl)methyl)pyridin-2-amine/6-bromo-3-chloro-N-((tetrahydro-2H-pyran-4-yl)methyl)pyridin-2-amine (5 g, ratio ˜2:3).

### Step 2: Preparation of 6-bromo-3,5-dichloro-N-((tetrahydro-2H-pyran-4-yl)methyl)-pyridin-2-amine

To a solution of a mixture of 6-bromo-3,5-dichloro-N-((tetrahydro-2H-pyran-4-yl)methyl)pyridin-2-amine/6-bromo-3-chloro-N-((tetrahydro-2H-pyran-4-yl)methyl)pyridin-2-amine (4.5 g, ratio ˜2:3) in acetonitrile (40 mL) was added N-chlorosuccinimide (1.25 g, 9.36 mmol). The mixture was heated to 80° C. for 50 min, cooled to room temperature and concentrated under reduced pressure. The residue was purified by column chromatography [silica gel, 120 g, EtOAc/heptane] providing 6-bromo-3,5-dichloro-N-((tetrahydro-2H-pyran-4-yl)methyl)pyridin-2-amine (2.25 g) as white solid. LCMS (m/z): 340.9 [M+H]+; Rt=1.11 min.

### Synthesis of 3,5,5′-trichloro-N6-((tetrahydro-2H-pyran-4-yl)methyl)-2,4′-bipyridine-2′,6-diamine

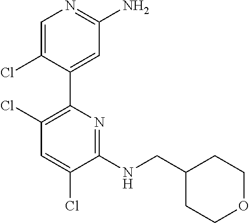

### Step 1: Preparation of 3,5,5′-trichloro-2′-fluoro-N-((tetrahydro-2H-pyran-4-yl)methyl)-2,4′-bipyridin-6-amine

A mixture of 6-bromo-3,5-dichloro-N-((tetrahydro-2H-pyran-4-yl)methyl)pyridin-2-amine (1 g, 2.94 mmol), 5-chloro-2-fluoropyridin-4-ylboronic acid (0.774 g, 4.41 mmol), PdCl₂(dppf) CH₂Cl₂ adduct (0.240 g, 0.294 mmol) in DME (12 mL) and 2M aqueous sodium carbonate solution (4 mL) in a sealed tube was heated at 90° C. for 2 hrs. The mixture was cooled to room temperature, diluted with EtOAc (˜100 mL) and saturated aqueous sodium bicarbonate solution. The separated organic layer was washed with saturated aqueous sodium bicarbonate solution (2×), brine, dried over sodium sulfate, filtered off and concentrated under reduced pressure. The residue was purified by column chromatography [silica gel, 80 g, EtOAc/heptane=0/100 to 30/70 over 25 min] providing 3,5,5′-trichloro-2′-fluoro-N-((tetrahydro-2H-pyran-4-yl)methyl)-2,4′-bipyridin-6-amine (510 mg) as a colorless liquid. LCMS (m/z): 391.9 [M+H]+; Rt=1.14 min.

### Step 2: Preparation of 3,5,5′-trichloro-N6-((tetrahydro-2H-pyran-4-yl)methyl)-2,4′-bipyridine-2′,6-diamine

A mixture of 3,5,5′-trichloro-2′-fluoro-N-((tetrahydro-2H-pyran-4-yl)methyl)-2,4′-bipyridin-6-amine (450 mg, 1.152 mmol) and ammonium hydroxide (aqueous solution 30-35 wt. %, 10 mL) in DMSO (10 mL) was heated in a steel bomb at 135° C. for 16 hrs. The mixture was cooled to room temperature and diluted with EtOAc and brine. The separated organic layer was washed with saturated aqueous sodium bicarbonate solution, dried over sodium sulfate, filtered off and concentrated under reduced pressure. The crude material of 3,5,5′-trichloro-N6-((tetrahydro-2H-pyran-4-yl)methyl)-2,4′-bipyridine-2′,6-diamine (480 mg) was directly used in the next reaction without further purification. LCMS (m/z): 387.1/389.1 [M+H]+; Rt=0.73 min.

### Synthesis of 5′-chloro-3-fluoro-N6-((tetrahydro-2H-pyran-4-yl)methyl)-2,4′-bipyridine-2′,6-diamine

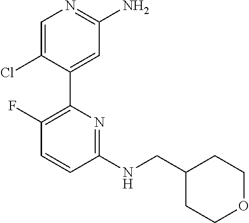

### Step 1. Preparation of 3,6-difluoro-2-methoxypyridine

To a solution of 2,3,6-trifluoropyridine (17.91 mL, 188 mmol) in anhydrous MeOH (300 mL) under argon was added sodium methoxide (25 wt. % in MeOH, 43 mL). The reaction mixture was heated at 65° C. for 2 hrs, cooled to room temperature, and concentrated under reduced pressure. The residue was diluted with brine (200 mL) and extracted with diethylether (3×200 mL). The combined organic extracts were dried over sodium sulphate, filtered off and concentrated under reduced pressure to give crude 3,6-difluoro-2-methoxypyridine (21.5 g) as a white solid, which was carried on to the next step without purification.

### Step 2. Preparation of 3,6-difluoro-2-hydroxypyridine

To a solution of 3,6-difluoro-2-methoxypyridine (21.5 g, 148 mmol) in acetonitrile (250 mL) was added sodium iodide (66.6 g, 445 mmol) and chlorotrimethylsilane (56.8 mL, 445 mmol) was heated at 80-85° C. for 2.5 hrs. The mixture was cooled to room temperature and diluted with EtOAc (300 mL) and water (300 mL), and vigorously stirred for 1 hr. The layers were separated and the aqueous phase was extracted with EtOAc (200 mL). The combined organic layers were washed sequentially with 0.6N aqueous hydrochloride solution (250 mL) and brine (250 mL) and concentrated under reduced pressure. The residue was filtered and rinsed three times with cold acetonitrile to give 3,6-difluoro-2-hydroxypyridine (10.8 g) as a white solid. The filtrate was concentrated and purified by column chromatography [silica gel, EtOAc/heptane] to give additional 3,6-difluoro-2-hydroxypyridine (4.2 g). LCMS (m/z): 132.0 [M+H]+; Rt=0.47 min.

### Step 3: Preparation of 3,6-difluoropyridin-2-yl trifluoromethanesulfonate

To an ice water bath-cooled solution of 3,6-difluoro-2-hydroxypyridine (10.75 g, 82 mmol) and triethylamine (22.86 mL, 164 mmol) in dichloromethane (550 mL) was added a solution of trifluoromethanesulfonic anhydride (16.63 mL, 98 mmol) in dichloromethane (100 mL) over 20 min. The resulting mixture was stirred for 2 hrs at 0° C. and diluted with saturated aqueous sodium bicarbonate solution (200 mL). The separated aqueous layer was extracted with dichloromethane (2×). The combined organic layers were dried over sodium sulphate, filtered, and concentrated under reduced pressure. The residue was purified by column chromatography [silica gel, EtOAc/heptane] to give 3,6-difluoropyridin-2-yl trifluoromethanesulfonate (16.3 g).

### Step 4. Preparation of 5′-chloro-2′,3,6-trifluoro-2,4′-bipyridine

A mixture of 3,6-difluoropyridin-2-yl trifluoromethanesulfonate (3.50 g, 13.30 mmol) and 5-chloro-2-fluoropyridine-4-boronic acid (3.27 g, 18.62 mmol) in tetrahydrofuran (27 mL) was degassed by purging argon through the mixture for 10 min. A 2M aqueous sodium carbonate solution (13.30 mL, 26.6 mmol) and PdCl₂(dppf) CH₂Cl₂ adduct (0.652 g, 0.798 mmol) were added, and the mixture was degassed for an additional 5 min. The reaction mixture was stirred at 100° C. for 2 hrs in a sealed vessel. The reaction mixture was cooled and diluted with EtOAc and water. The separated organic layer was dried over sodium sulphate, filtered, and concentrated under reduced pressure. The residue was purified by column chromatography [silica gel, EtOAc/heptane] providing of 5′-chloro-2′,3,6-trifluoro-2,4′-bipyridine (2.78 g) as a solid. LCMS (m/z): 244.9 [M+H]+; Rt=0.86 min.

### Step 5. Preparation of 5′-chloro-3,6-difluoro-2,4′-bipyridin-2′-amine

A mixture of 5′-chloro-2′,3,6-trifluoro-2,4′-bipyridine (220 mg, 0.899 mmol) and saturated aqueous ammonium hydroxide solution (3 mL, 21.57 mmol) in DMSO (3 mL) was heated in a steel bomb at 120° C. for 17 hrs. The mixture was allowed to cool to room temperature and was diluted with water and extracted with EtOAc. The combined organic layers were washed with brine, dried over sodium sulphate, filtered off and concentrated under reduced pressure providing crude 5′-chloro-3,6-difluoro-2,4′-bipyridin-2′-amine (220 mg), which was directly used in the next step without further purification. LCMS (m/z): 241.9 [M+H]+, Rt=0.52 min.

### Step 6. Preparation of 5′-chloro-3-fluoro-N6-((tetrahydro-2H-pyran-4-yl)methyl)-2,4′-bipyridine-2′,6-diamine

A mixture of 5′-chloro-3,6-difluoro-2,4′-bipyridin-2′-amine (220 mg, 0.637 mmol) and 4-aminomethyltetrahydropyran (441 mg, 3.82 mmol) in DMSO (3 mL) was irradiated at 180° C. for 30 min and at 190° C. for 15 min. The mixture was cooled to room temperature, diluted with water and extracted with EtOAc. The combined organic extracts were washed with water and brine, dried over sodium sulphate, filtered and concentrated under reduced pressure. The residue was purified by column chromatography [silica gel, DCM/MeOH with 1% of triethylamine) providing 5′-chloro-3-fluoro-N6-((tetrahydro-2H-pyran-4-yl)methyl)-2,4′-bipyridine-2′,6-diamine (118 mg) as an off-white solid. LCMS (m/z): 337.1 [M+H]+, Rt=0.56 min.

### Synthesis of 5-fluoro-6-(((tetrahydro-2H-pyran-4-yl)methyl)amino)pyridin-2-yl trifluoromethanesulfonate

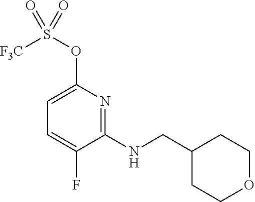

### Step 1: Preparation of 3,6-difluoro-N-((tetrahydro-2H-pyran-4-yl)methyl)pyridin-2-amine

A mixture of 2,3,6-trifluoropyridine (3 g, 22.54 mmol), (tetrahydro-2H-pyran-4-yl)methanamine (3.89 g, 33.8 mmol) and triethylamine (7.86 mL, 56.4 mmol) in NMP (60 mL) was heated at 70° C. for 1 hr. The reaction mixture was cooled to room temperature, diluted with EtOAc (˜100 mL), brine (˜50 mL) and water (˜50 mL). The separated organic layer was washed with brine (1×), 0.3N aqueous hydrochloride solution (2×), saturated aqueous sodium bicarbonate solution (1×), brine (1×), dried over sodium sulfate, filtered and concentrated under reduced pressure providing crude 3,6-difluoro-N-((tetrahydro-2H-pyran-4-yl)methyl)pyridin-2-amine (3.5 g), which was directly used in the next reaction without further purification. LCMS (m/z): 229.1 [M+H]+; Rt=0.79 min.

### Step 2: Preparation of 3-fluoro-6-methoxy-N-((tetrahydro-2H-pyran-4-yl)methyl)pyridin-2-amine

To a solution of 3,6-difluoro-N-((tetrahydro-2H-pyran-4-yl)methyl)pyridin-2-amine (5 g, 21.91 mmol) in MeOH (35 mL) was added sodium methoxide (25 wt. % in MeOH, 15.03 mL). The mixture was heated in a steel bomb at 135° C. for ˜18 hrs, cooled to room temperature and concentrated under reduced pressure. The residue was taken up in water (˜250 mL). The precipitate was filtered and rinsed with water. The solid was dissolved in toluene (10 mL)/dichloromethane (10 mL), decanted from the dark brownish film and concentrated under reduced pressure. The residue was dried in high vacuo providing crude 3-fluoro-6-methoxy-N-((tetrahydro-2H-pyran-4-yl)methyl)pyridin-2-amine (4.96 g), which was directly used in the next reaction without further purification. LCMS (m/z): 241.1 [M+H]+; Rt=0.87 min.

### Step 3: Preparation of 5-fluoro-6-(((tetrahydro-2H-pyran-4-yl)methyl)amino)pyridin-2-ol

To a solution of 3-fluoro-6-methoxy-N-((tetrahydro-2H-pyran-4-yl)methyl)pyridin-2-amine (4.6 g, 19.14 mmol) in acetonitrile (50 mL) was added sodium iodide (20.09 g, 134 mmol) and chlorotrimethylsilane (17.13 mL, 134 mmol). The mixture was stirred at 95° C. for 20 hrs, cooled to room temperature, diluted with EtOAc (80 mL) and water (40 mL). The mixture was stirred vigorously for 30 min. The separated organic layer was washed with 0.1N aqueous hydrochloride solution. The combined aqueous layers were carefully neutralized (pH ˜7) with solid sodium bicarbonate solution and extracted with EtOAc (1×100 mL) and dichloromethane (2×50 mL). The organic layers were washed with saturated aqueous sodium bicarbonate solution and brine, dried over sodium sulfate, filtered off and concentrated under reduced pressure. The residue was purified by column chromatography [silica gel, 80 g, EtOAc/heptane=10/90 to 100/0] providing 5-fluoro-6-(((tetrahydro-2H-pyran-4-yl)methyl)amino)pyridin-2-ol (780 mg). LCMS (m/z): 227.1 [M+H]+; Rt=0.42 min.

### Step 4: Preparation of 5-fluoro-6-((tetrahydro-2H-pyran-4-yl)methyl)amino)pyridin-2-yl trifluoromethanesulfonate

To a solution of 5-fluoro-6-((tetrahydro-2H-pyran-4-yl)methylamino)pyridin-2-ol (500 mg, 2.210 mmol) and triethylamine (0.462 mL, 3.31 mmol) in dichloromethane (20 mL) was slowly added trifluoromethanesulfonic anhydride (1.120 mL, 6.63 mmol) at 0° C. The mixture was stirred for 2 hrs at 0° C. and poured carefully into ice-cooled saturated aqueous sodium bicarbonate solution. The separated aqueous layer was extracted with dichloromethane (2×). The combined organic layers were dried over sodium sulfate, filtered off and concentrated under reduced pressure. The residue was purified by column chromatography [silica gel, 40 g, EtOAc/heptane=5/95 to 40/60] providing 5-fluoro-6-(((tetrahydro-2H-pyran-4-yl)methyl)amino)pyridin-2-yl trifluoromethanesulfonate (743 mg) as colorless oil. LCMS (m/z): 359.0 [M+H]+; Rt=1.02 min.

### Synthesis of 5′-chloro-5-fluoro-N6-((tetrahydro-2H-pyran-4-yl)methyl)-2,4′-bipyridine-2′,6-diamine

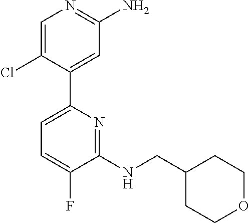

### Step 1: Preparation of 5′-chloro-2′,5-difluoro-N-((tetrahydro-2H-pyran-4-yl)methyl)-2,4′-bipyridin-6-amine

A mixture of 5-fluoro-6-((tetrahydro-2H-pyran-4-yl)methylamino)pyridin-2-yl trifluoromethanesulfonate (712 mg, 1.987 mmol), 5-chloro-2-fluoropyridin-4-ylboronic acid (697 mg, 3.97 mmol), PdCl₂(dppf) CH₂Cl₂ adduct (162 mg, 0.199 mmol) in DME (8 mL) and 2M aqueous sodium carbonate solution (2.6 mL, 1.987 mmol) was heated in a sealed tube at 95° C. for 3 hrs. The mixture was cooled to room temperature and diluted with EtOAc (˜100 mL) and saturated aqueous sodium bicarbonate carbonate solution. The separated organic layer was washed with saturated aqueous sodium bicarbonate carbonate (2×), dried over sodium sulfate, filtered off and concentrated under reduced pressure. The residue was purified by column chromatography [silica gel, 40 g, EtOAc/heptane=0/100 to 25/75] providing 5′-chloro-2′,5-difluoro-N-((tetrahydro-2H-pyran-4-yl)methyl)-2,4′-bipyridin-6-amine (570 mg) as a white solid. LCMS (m/z): 340.1 [M+H]+; Rt=0.99 min.

### Step 2: Preparation of 5′-chloro-5-fluoro-N6-((tetrahydro-2H-pyran-4-yl)methyl)-2,4′-bipyridine-2′,6-diamine

A mixture of 5′-chloro-2′,5-difluoro-N-((tetrahydro-2H-pyran-4-yl)methyl)-2,4′-bipyridin-6-amine (450 mg, 1.324 mmol) and ammonium hydroxide (aqueous solution 30-35 wt. %, 12 mL) in DMSO (12 mL) was heated in a steel bomb at 135° C. for 16 hrs. The mixture was cooled to room temperature and was diluted with EtOAc and brine. The separated organic layer was washed with saturated aqueous sodium bicarbonate solution, dried over sodium sulfate, filtered and concentrated under reduced pressure providing crude 5′-chloro-5-fluoro-N6-((tetrahydro-2H-pyran-4-yl)methyl)-2,4′-bipyridine-2′,6-diamine, which was directly used in the next reaction without further purification. LCMS (m/z): 337.1 [M+H]+; Rt=0.59 min.

### Synthesis of 3,5′-dichloro-5-fluoro-N6-((tetrahydro-2H-pyran-4-yl)methyl)-2,4′-bipyridine-2′,6-diamine

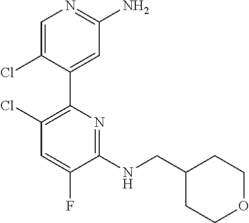

### Step 1: Preparation of 3,5′-dichloro-2′,5-difluoro-N-((tetrahydro-2H-pyran-4-yl)methyl)-2,4′-bipyridin-6-amine

To a solution of 3,5′-dichloro-2′-fluoro-N-((tetrahydro-2H-pyran-4-yl)methyl)-2,4′-bipyridin-6-amine (900 mg, 2.53 mmol) in acetonitrile (10 mL) was added 1-(chloromethyl)-4-fluoro-1,4-diazoniabicyclo[2.2.2]octane tetrafluoroborate (Selectfluor) (1343 mg, 3.79 mmol). The mixture was stirred at 25° C. for 22 hrs, cooled to ambient temperature, diluted with EtOAc (50 mL) and saturated aqueous sodium bicarbonate solution (50 mL). The separated organic layer was washed with saturated aqueous sodium bicarbonate solution (2×), dried over sodium sulfate, filtered off and concentrated under reduced pressure. The residue was purified by column chromatography providing 3,5′-dichloro-2′,5-difluoro-N-((tetrahydro-2H-pyran-4-yl)methyl)-2,4′-bipyridin-6-amine (70 mg). LCMS (m/z): 373.9/376.0 [M+H]+; Rt=1.12 min.

### Step 2: Preparation of 3,5′-dichloro-5-fluoro-N6-((tetrahydro-2H-pyran-4-yl)methyl)-2,4′-bipyridine-2′,6-diamine

A mixture of 3,5′-dichloro-2′,5-difluoro-N-((tetrahydro-2H-pyran-4-yl)methyl)-2,4′-bipyridin-6-amine (70 mg, 0.187 mmol) and ammonium hydroxide (aqueous solution 30-35 wt. %, 3 mL) in DMSO (3 mL) was heated in a steel bomb at 110° C. for 18 hrs. The mixture was cooled to room temperature and was diluted with dichloromethane and water. The separated organic layer was washed with water and brine, dried over sodium sulfate, filtered and concentrated under reduced pressure. The residue was dissolved in acetonitrile/water and lyophilized providing crude 3,5′-dichloro-5-fluoro-N6-((tetrahydro-2H-pyran-4-yl)methyl)-2,4′-bipyridine-2′,6-diamine (68 mg), which was directly used in the next reaction without further purification. LCMS (m/z): 371.0/373.0 [M+H]+; Rt=0.67 min.

### Synthesis of 6-chloro-N-((tetrahydro-2H-pyran-4-yl)methyl)-5-(trifluoromethyl)pyridin-2-amine and 6-chloro-N-((tetrahydro-2H-pyran-4-yl)methyl)-3-(trifluoromethyl)pyridin-2-amine

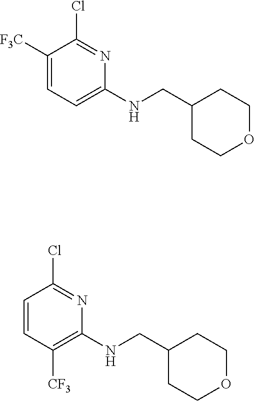

To a solution of 2,6-dichloro-3-(trifluoromethyl)pyridine (320 mg, 1.48 mmol) in DMSO (1.5 mL) at room temperature was added (tetrahydro-2H-pyran-4-yl)methanamine (188 mg, 1.63 mmol) and triethylamine (0.207 mL, 1.48 mmol). The mixture was heated at 120° C. in a sealed glass bomb for 18 hrs. The reaction mixture was diluted with EtOAc (20 mL) and the organic layer was washed with saturated aqueous sodium bicarbonate solution and brine, dried over sodium sulfate, filtered off and concentrated under reduced pressure. The crude material was purified by column chromatography [silica gel, 120 g, EtOAc/hexane=10/90 to 50/50] providing 6-chloro-N-((tetrahydro-2H-pyran-4-yl)methyl)-5-(trifluoromethyl)pyridin-2-amine (340 mg) {LCMS (m/z): 295.2 [M+H]+; Rt=0.97 min} and 6-chloro-N-((tetrahydro-2H-pyran-4-yl)methyl)-3-(trifluoromethyl)pyridin-2-amine (80 mg) {LCMS (m/z): 295.1 [M+H]+; Rt=1.03 min}.

### Synthesis of 5′-chloro-N6-((tetrahydro-2H-pyran-4-yl)methyl)-5-(trifluoromethyl)-2,4′-bipyridine-2′,6-diamine

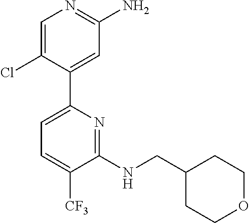

### Step 1: Preparation of 5′-chloro-2′-fluoro-N-((tetrahydro-2H-pyran-4-yl)methyl)-5-(trifluoromethyl)-2,4′-bipyridin-6-amine

A mixture of 6-chloro-N-((tetrahydro-2H-pyran-4-yl)methyl)-3-(trifluoromethyl)pyridin-2-amine (80 mg, 0.271 mmol), 5-chloro-2-fluoropyridin-4-ylboronic acid (89 mg, 0.509 mmol), PdCl₂(dppf) CH₂Cl₂ adduct (27.7 mg, 0.034 mmol) in DME (1.5 mL) and 2M aqueous sodium carbonate solution (0.5 mL, 1 mmol) in a sealed tube was heated at 100° C. for 3 hrs. The mixture was allowed to cool to room temperature and was diluted with EtOAc (25 mL), filtered and concentrated under reduced pressure. The residue was purified by column chromatography [silica gel, 12 g, EtOAc/heptane=5/100 to 50/50]. Fractions were combined and concentrated under reduced pressure providing 5′-chloro-2′-fluoro-N-((tetrahydro-2H-pyran-4-yl)methyl)-5-(trifluoromethyl)-2,4′-bipyridin-6-amine (97 mg). LCMS (m/z): 390.2 [M+H]+; Rt=1.12 min.

### Step 2: Preparation of 5′-chloro-N6-((tetrahydro-2H-pyran-4-yl)methyl)-5-(trifluoromethyl)-2,4′-bipyridine-2′,6-diamine

A mixture of 5′-chloro-2′-fluoro-N-((tetrahydro-2H-pyran-4-yl)methyl)-5-(trifluoromethyl)-2,4′-bipyridin-6-amine (67 mg, 0.172 mmol) and ammonium hydroxide (aqueous solution 30-35 wt. %, 1 mL) in DMSO (1 mL) was heated at 130° C. for ˜16 hrs. The mixture was cooled to room temperature and diluted with EtOAc. The organic layer was washed with water (3×10 mL) and dried over sodium sulfate, filtered and concentrated under reduced pressure. The crude material of 5′-chloro-N6-((tetrahydro-2H-pyran-4-yl)methyl)-5-(trifluoromethyl)-2,4′-bipyridine-2′,6-diamine (62 mg) was directly used in the next reaction without further purification. LCMS (m/z): 387.2 [M+H]+; Rt=0.73 min.

### Synthesis of 3-chloro-5′-fluoro-N6-((tetrahydro-2H-pyran-4-yl)methyl)-2,4′-bipyridine-2′,6-diamine

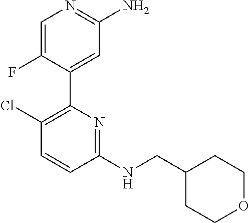

### Step 1: Preparation of 2,5-difluoropyridin-4-ylboronic acid

To a solution of diisopropylamine (1.74 mL, 12.20 mmol) in anhydrous tetrahydrofuran (22 mL) under argon at −20° C. was added n-butyllithium (7.66 mL, 1.6M in hexanes) slowly over 10 min. The newly formed LDA was then cooled to −78° C. A solution of 2,5-difluoropyridine (1.05 mL, 11.5 mmol) in anhydrous tetrahydrofuran (3 mL) was added slowly over 30 min and the mixture was stirred at −78° C. for 4 hrs. A solution of triisopropyl borate (5.90 mL, 25.4 mmol) in anhydrous tetrahydrofuran (8.6 mL) was added drop wise. Once the addition was complete the reaction mixture was warmed to room temperature and stirring was continued for an additional hour. The reaction mixture was diluted with aqueous sodium hydroxide solution (4 wt. %, 34 mL). The separated aqueous layer was cooled to 0° C. and then slowly acidified to pH=4 with 6N aqueous hydrochloride solution (˜10 mL). The mixture was extracted with EtOAc (3×50 mL). The combined organic layers washed with brine (50 mL), dried over sodium sulfate, filtered off and concentrated under reduced pressure. The residue was triturated with diethylether to give 2,5-difluoropyridin-4-ylboronic acid (808 mg).

### Step 2: Preparation of 3-chloro-2′,5′-difluoro-N-((tetrahydro-2H-pyran-4-yl)methyl)-2,4′-bipyridin-6-amine

A mixture of 6-bromo-5-chloro-N-((tetrahydro-2H-pyran-4-yl)methyl)pyridin-2-amine (0.500 g, 1.64 mmol), 2,5-difluoropyridin-4-ylboronic acid (0.260 g, 1.64 mmol) in DME (7.4 mL) and 2M aqueous sodium carbonate solution (2.45 mL, 4.9 mmol) was degassed with argon for 5 min. To the mixture was added PdCl₂(dppf) CH₂Cl₂ adduct (0.267 g, 0.327 mmol). The reaction mixture was heated in the microwave at 105° C. for 25 min. Additional boronic acid (0.260 g, 1.64 mmol) and PdCl₂(dppf) CH₂Cl₂ adduct (0.267 g, 0.327 mmol), and water (˜2 mL) were added and heating was continued at 110° C. for 30 min. The mixture was filtered through a pad of celites and the filtrate concentrated under reduced pressure. The residue was purified by column chromatography [silica gel, 40 g, EtOAc/heptane=10/90 to 80/20] providing 3-chloro-2′,5′-difluoro-N-((tetrahydro-2H-pyran-4-yl)methyl)-2,4′-bipyridin-6-amine (358 mg). LCMS (m/z): 340.0 [M+H]+; Rt=0.90 min. ¹H NMR (400 MHz, chloroform-d) d [ppm]: 1.37 (qd, 3H) 1.60 (br. s., 2H) 1.68 (d, J=12.91 Hz, 3H) 1.84 (ddd, J=11.15, 7.24, 4.30 Hz, 1H) 3.21 (t, J=6.26 Hz, 2H) 3.32-3.45 (m, 3H) 4.00 (dd, J=11.15, 3.72 Hz, 2H) 4.74 (br. s., 1H) 6.45 (d, J=9.00 Hz, 1H) 6.99-7.07 (m, 1H) 7.51 (d, J=8.61 Hz, 1H) 8.12 (s, 1H).

### Step 3: Preparation of 3-chloro-5′-fluoro-N6-((tetrahydro-2H-pyran-4-yl)methyl)-2,4′-bipyridine-2′,6-diamine

A mixture of 3-chloro-2′,5′-difluoro-N-((tetrahydro-2H-pyran-4-yl)methyl)-2,4′-bipyridin-6-amine (0.309 g, 0.889 mmol) and ammonium hydroxide (aqueous solution 30-35 wt. %, 8 mL) in DMSO (8 mL) was heated in a steel bomb at 135° C. for 18 hrs. After cooling to room temperature additional ammonium hydroxide (aqueous solution 30-35 wt. %, 5 mL) was added and heating at 155° C. was continued for 18 hrs. The mixture was allowed to cool to room temperature and was diluted with water. The mixture was extracted with EtOAc (3×50 mL). The combined organic layers were washed with brine (25 mL), dried over sodium sulfate, filtered off and concentrated under reduced pressure providing crude 3-chloro-5′-fluoro-N6-((tetrahydro-2H-pyran-4-yl)methyl)-2,4′-bipyridine-2′,6-diamine (309 mg), which was directly used in the next reaction without further purification. LCMS (m/z): 337.1 [M+H]+; Rt=0.59 min.

### Synthesis of N6-((tetrahydro-2H-pyran-4-yl)methyl)-2,4′-bipyridine-2′,6-diamine

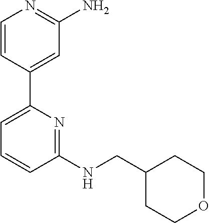

### Step 1: Preparation of 2′-fluoro-N-((tetrahydro-2H-pyran-4-yl)methyl)-2,4′-bipyridin-6-amine

A mixture of 6-bromo-N-((tetrahydro-2H-pyran-4-yl)methyl)pyridin-2-amine (400 mg, 1.48 mmol), 2-aminopyridin-4-ylboronic acid (312 mg, 2.21 mmol), PdCl₂(dppf) CH₂Cl₂ adduct (120 mg, 0.148 mmol) in DME (6.3 mL) and 2M aqueous sodium carbonate solution (2.102 mL, 4.20 mmol) was heated in a sealed tube at 103° C. for 16 hrs. The mixture was cooled to room temperature and was diluted with EtOAc (˜25 mL) and saturated aqueous. The separated organic layer was washed with saturated aqueous sodium bicarbonate solution (2×), dried over sodium sulfate, filtered off and concentrated under reduced pressure. The residue was purified by column chromatography [silica gel, 12 g, EtOAc/heptane=5/95 to 50/50] providing 2′-fluoro-N-((tetrahydro-2H-pyran-4-yl)methyl)-2,4′-bipyridin-6-amine (280 mg) as a colorless liquid, which became slowly a white solid. LCMS (m/z): 288.1.1 [M+H]+; Rt=0.53 min.

### Step 2: Preparation of N6-((tetrahydro-2H-pyran-4-yl)methyl)-2,4′-bipyridine-2′,6-diamine

A mixture of 2′-fluoro-N-((tetrahydro-2H-pyran-4-yl)methyl)-2,4′-bipyridin-6-amine (450 mg, 1.152 mmol) and ammonium hydroxide (aqueous solution 30-35 wt. %, 4 mL) in DMSO (3 mL) was heated in a steel bomb at 135° C. for 16 hrs. The mixture was cooled to room temperature and was diluted with EtOAc and brine. The separated organic layer was washed with saturated aqueous sodium bicarbonate solution, dried over sodium sulfate, filtered and concentrated under reduced pressure. The crude material of N6-((tetrahydro-2H-pyran-4-yl)methyl)-2,4′-bipyridine-2′,6-diamine (222 mg) was directly used in the next reaction without further purification. LCMS (m/z): 285.1 [M+H]+; Rt=0.41 min.

### Synthesis of (S)-6-bromo-5-chloro-N-(1-(tetrahydro-2H-pyran-4-yl)ethyl)pyridin-2-amine

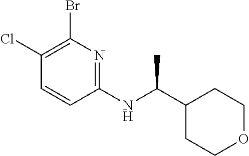

### Step 1: Preparation of (R,E)-2-methyl-N-((tetrahydro-2H-pyran-4-yl)methylene)propane-2-sulfinamide

A mixture of tetrahydro-2H-pyran-4-carbaldehyde (2.0 g, 17.52 mmol), (R)-2-methylpropane-2-sulfinamide (1.062 g, 8.76 mmol), pyridine 4-methylbenzenesulfonate (0.110 g, 0.438 mmol) and magnesium sulfate (5.27 g, 43.8 mmol) in dichloroethane (13 mL) was stirred at room temperature for 18 hrs. The solids were filtered off and the filtrate was concentrated to dryness under reduced pressure. The residue was purified by column chromatography [silica gel] providing (R,E)-2-methyl-N-((tetrahydro-2H-pyran-4-yl)methylene)propane-2-sulfinamide (1.9 g). LCMS (m/z): 218.1 [M+H]+; Rt=0.58 min.

### Step 2: Preparation of (R)-2-methyl-N—((S)-1-(tetrahydro-2H-pyran-4-yl)ethyl)propane-2-sulfinamide

To a solution of (R,E)-2-methyl-N-((tetrahydro-2H-pyran-4-yl)methylene)propane-2-sulfinamide (0.93 g, 4.28 mmol) in dichloromethane (21.4 mL) at 0° C. was added slowly methylmagnesium bromide (2.0 M in tetrahydrofuran, 4.28 mL, 8.56 mmol). The reaction mixture was warmed to room temperature and stirred for 3 hrs. The mixture was diluted with saturated aqueous ammonium chloride solution (5 mL). The separated organic layer was washed with water and brine, dried over sodium sulfate and concentrated to dryness under reduced pressure. The residue was purified by column chromatography providing (R)-2-methyl-N—((S)-1-(tetrahydro-2H-pyran-4-yl)ethyl)propane-2-sulfinamide (910 mg). LCMS (m/z): 234.0 [M+H]+; Rt=0.58 min.

### Step 3: Preparation of (S)-1-(tetrahydro-2H-pyran-4-yl)ethanamine

To a solution of (R)-2-methyl-N—((S)-1-(tetrahydro-2H-pyran-4-yl)ethyl)propane-2-sulfinamide (400 mg, 1.714 mmol) in MeOH (5 mL) was added 4M hydrochloride in dioxane (5 mL). The reaction mixture was stirred at room temperature for 30 min. The mixture was concentrated under reduced pressure and the residue was diluted with diethylether (10 mL). The precipitate was collected by filtration and washed with diethylether providing crude (S)-1-(tetrahydro-2H-pyran-4-yl)ethanamine hydrochloride salt. The hydrochloride salt was dissolved in water (10 mL) and neutralized with saturated aqueous sodium bicarbonate solution. The mixture was extracted with dichloromethane. The organic layer was dried over sodium sulfate, filtered off and concentrated under reduced pressure providing crude (S)-1-(tetrahydro-2H-pyran-4-yl)ethanamine (212 mg), which was directly used in the next reaction without further purification. LCMS (m/z): 130.1 [M+H]+; Rt=0.34 min.

### Step 4: Preparation of (S)-6-bromo-N-(1-(tetrahydro-2H-pyran-4-yl)ethyl)pyridin-2-amine

A mixture of 2-bromo-6-fluoropyridine (225 mg, 1.280 mmol), (S)-1-(tetrahydro-2H-pyran-4-yl)ethanamine (212 mg, 1.280 mmol), DIPEA (331 g, 2.5 mmol) and DMSO (5 mL) was heated in a sealed tube at 90° C. for 18 hrs. The reaction mixture was cooled to room temperature, poured into water (30 mL) and stirred for 20 min. The mixture was extracted with EtOAc (3×15 mL). The combined organic layers were washed with brine (100 mL) and concentrated to dryness under reduced pressure. The residue was purified by column chromatography [silica gel] providing (S)-6-bromo-N-(1-(tetrahydro-2H-pyran-4-yl)ethyl)pyridin-2-amine (270 mg). LCMS (m/z): 285.0/286.9 [M+H]+; Rt=0.91 min.

### Step 5: Preparation of (S)-6-bromo-5-chloro-N-(1-(tetrahydro-2H-pyran-4-yl)ethyl)pyridin-2-amine

To a solution of (S)-6-bromo-N-(1-(tetrahydro-2H-pyran-4-yl)ethyl)pyridin-2-amine (236 mg, 0.828 mmol) in acetonitrile (5 mL) was added N-chlorosuccinimide (111 mg, 0.828 mmol), and the resulting mixture was heated at 80° C. for 3 hrs. The reaction mixture was allowed to cool to 25° C. and concentrated under reduced pressure. The residue was diluted with brine (20 mL) and extracted with EtOAc (3×20 mL). The combined organic layers were dried over sodium sulfate, filtered off and before concentrated under reduced pressure. The residue was purified by column chromatography providing (S)-6-bromo-5-chloro-N-(1-(tetrahydro-2H-pyran-4-yl)ethyl)pyridin-2-amine (190 mg). LCMS (m/z): 318.9/320.9 [M+H]+; Rt=1.08 min.

### Synthesis of (S)-3-chloro-N6-(1-(tetrahydro-2H-pyran-4-yl)ethyl)-2,4′-bipyridine-2′,6-diamine

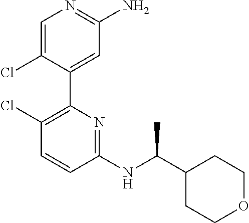

### Step 1: Preparation of (S)-3,5′-dichloro-2′-fluoro-N-(1-(tetrahydro-2H-pyran-4-yl)ethyl)-2,4′-bipyridin-6-amine

A mixture of (S)-6-bromo-5-chloro-N-(1-(tetrahydro-2H-pyran-4-yl)ethyl)pyridin-2-amine (290 mg, 0.907 mmol), 2-amino-5-chloropyridin-4-ylboronic acid (318 mg, 1.815 mmol), PdCl₂(dppf) CH₂Cl₂ adduct (59.3 mg, 0.073 mmol) in DME (4 mL) and 2M aqueous sodium carbonate solution (1.43 mL, 2.85 mmol) was heated at 90° C. for 2 hrs. The reaction mixture was allowed to cool to room temperature and concentrated to dryness under reduced pressure. The residue was diluted with EtOAc. The mixture was washed with saturated aqueous sodium bicarbonate solution and brine. The organic layer was dried over sodium sulfate, filtered off and concentrated under reduced pressure. The residue was purified by column chromatography [silica gel]providing (S)-3,5′-dichloro-2′-fluoro-N-(1-(tetrahydro-2H-pyran-4-yl)ethyl)-2,4′-bipyridin-6-amine (260 mg). LCMS (m/z): 369.9/371.8 [M+H]+; Rt=1.01 min.

### Step 2: Preparation of (S)-3,5′-dichloro-N6-(1-(tetrahydro-2H-pyran-4-yl)ethyl)-2,4′-bipyridine-2′,6-diamine

A mixture of (S)-3,5′-dichloro-2′-fluoro-N-(1-(tetrahydro-2H-pyran-4-yl)ethyl)-2,4′-bipyridin-6-amine (230 mg, 0.621 mmol) and ammonium hydroxide (aqueous solution 30-35 wt. %, 5 mL) in DMSO (5 mL) was heated in a steel bomb at 110° C. for 18 hrs. The mixture was cooled to room temperature and was diluted with dichloromethane and water. The separated organic layer was washed with water, dried over sodium sulfate, filtered off and concentrated under reduced pressure. The residue was dissolved in acetonitrile/water and lyophilized providing crude (S)-3,5′-dichloro-N-6-(1-(tetrahydro-2H-pyran-4-yl)ethyl)-2,4′-bipyridine-2′,6-diamine (220 mg), which was directly used in the next reaction without further purification. LCMS (m/z): 367.1/369.1 [M+H]+; Rt=0.95 min.

### Synthesis of (R)-6-bromo-5-chloro-N-(1-(tetrahydro-2H-pyran-4-yl)ethyl)pyridin-2-amine

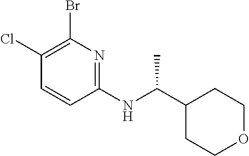

### Step 1: Preparation of (S,E)-2-methyl-N-((tetrahydro-2H-pyran-4-yl)methylene)propane-2-sulfinamide

A mixture of tetrahydro-2H-pyran-4-carbaldehyde (2.0 g, 17.52 mmol), (S)-2-methylpropane-2-sulfinamide (1.062 g, 8.76 mmol), pyridine 4-methylbenzenesulfonate (0.110 g, 0.438 mmol) and magnesium sulfate (5.27 g, 43.8 mmol) in dichloroethane (13 mL) was stirred at room temperature for 18 hrs. The solids were filtered off and the filtrate was concentrated to dryness under reduced pressure. The residue was purified by column chromatography [silica gel] providing (S,E)-2-methyl-N-((tetrahydro-2H-pyran-4-yl)methylene)propane-2-sulfinamide (1.50 g). LCMS (m/z): 218.1 [M+H]+; Rt=0.58 min.

### Step 2: Preparation of (S)-2-methyl-N—((R)-1-(tetrahydro-2H-pyran-4-yl)ethyl)propane-2-sulfinamide

To a solution of (S,E)-2-methyl-N-((tetrahydro-2H-pyran-4-yl)methylene)propane-2-sulfinamide (1.5 g, 6.90 mmol) in dichloromethane (34.5 mL) at 0° C. was slowly added methylmagnesium bromide (1.646 g, 13.80 mmol). The reaction mixture was warmed to room temperature and stirred for 3 hrs. The mixture was diluted with saturated aqueous ammonium chloride solution (5 mL). The separated organic layer was washed with water and brine, dried over sodium sulfate and concentrated to dryness under reduced pressure. The residue was purified by column chromatograph providing (S)-2-methyl-N—((R)-1-(tetrahydro-2H-pyran-4-yl)ethyl)propane-2-sulfinamide (1.40 g). LCMS (m/z): 234.3 [M+H]+; Rt=0.57 min.

### Step 3: Preparation of (R)-1-(tetrahydro-2H-pyran-4-yl)ethanamine

To a solution of (S)-2-methyl-N—((R)-1-(tetrahydro-2H-pyran-4-yl)ethyl)propane-2-sulfinamide (400 mg, 1.714 mmol) in MeOH (5 mL) was added 4M hydrochloride in dioxane (5 mL). The reaction mixture was stirred at room temperature for 30 min. The mixture was concentrated under reduced pressure and the residue was diluted with diethylether (10 mL). The precipitate was collected by filtration and washed with diethylether providing crude (R)-1-(tetrahydro-2H-pyran-4-yl)ethanamine hydrochloride salt. The hydrochloride salt was dissolved in water (10 mL) and neutralized with saturated aqueous sodium bicarbonate solution. The mixture was extracted with dichloromethane (2×). The combined organic layers were dried over sodium sulfate, filtered off and concentrated under reduced pressure providing crude (R)-1-(tetrahydro-2H-pyran-4-yl)ethanamine (200 mg), which was directly used in the next reaction without further purification. LCMS (m/z): 130.1 [M+H]+; Rt=0.34 min.

### Step 4: Preparation of (R)-6-bromo-N-(1-(tetrahydro-2H-pyran-4-yl)ethyl)pyridin-2-amine

A mixture of 2-bromo-6-fluoropyridine (212 mg, 1.21 mmol), (R)-1-(tetrahydro-2H-pyran-4-yl)ethanamine (200 mg, 1.21 mmol), DIPEA (187 mg, 1.45 mmol) and DMSO (3 mL) was heated in a sealed tube at 90° C. for 18 hrs. The reaction mixture was allowed to cool to room temperature, poured into water (30 mL) and stirred for 20 min. The mixture was extracted with EtOAc (3×15 mL). The combined organic layers were washed with brine (100 mL) and concentrated to dryness under reduced pressure. The residue was purified by column chromatography [silica gel] providing (R)-6-bromo-N-(1-(tetrahydro-2H-pyran-4-yl)ethyl)pyridin-2-amine (290 mg). LCMS (m/z): 285.0/286.9 [M+H]+; Rt=0.91 min.

### Step 5: Preparation of (R)-6-bromo-5-chloro-N-(1-(tetrahydro-2H-pyran-4-yl)ethyl)pyridin-2-amine

To a solution of (R)-6-bromo-N-(1-(tetrahydro-2H-pyran-4-yl)ethyl)pyridin-2-amine (200 mg, 0.701 mmol) in acetonitrile (5 mL) was added N-chlorosuccinimide (94 mg, 0.701 mmol). The mixture was heated at 80° C. for 3 hrs. The reaction mixture was cooled to 25° C. and concentrated under reduced pressure. The residue was diluted with brine (20 mL) and extracted with EtOAc (3×20 mL). The combined organic layers were dried over sodium sulfate, filtered off and concentrated under reduced pressure. The residue was purified by column chromatography [silica gel] providing (R)-6-bromo-5-chloro-N-(1-(tetrahydro-2H-pyran-4-yl)ethyl)pyridin-2-amine (181 mg). LCMS (m/z): 318.9/320.9 [M+H]+; Rt=1.08 min.

### Synthesis of (R)-3,5′-dichloro-N6-(1-(tetrahydro-2H-pyran-4-yl)ethyl)-2,4′-bipyridine-2′,6-diamine

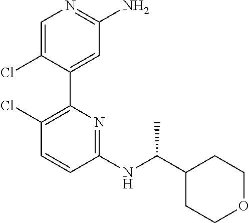

### Step 1: Preparation of (R)-3,5′-dichloro-2′-fluoro-N-(1-(tetrahydro-2H-pyran-4-yl)ethyl)-2,4′-bipyridin-6-amine

A mixture of (R)-6-bromo-5-chloro-N-(1-(tetrahydro-2H-pyran-4-yl)ethyl)pyridin-2-amine (350 mg, 1.10 mmol), 2-amino-5-chloropyridin-4-ylboronic acid (384 mg, 2.19 mmol), PdCl₂(dppf) CH₂Cl₂ adduct (71.5 mg, 0.088 mmol) in DME (5 mL) and 2M aqueous sodium carbonate solution (1.43 mL, 2.85 mmol) was heated at 90° C. for 2 hrs. The reaction mixture was cooled to room temperature and concentrated to dryness under reduced pressure. The residue was diluted with EtOAc. The mixture was washed with saturated aqueous sodium bicarbonate solution and brine. The organic layer was dried over sodium sulfate, filtered off and concentrated under reduced pressure. The residue was purified by column chromatography [silica gel] providing (R)-3,5′-dichloro-2′-fluoro-N-(1-(tetrahydro-2H-pyran-4-yl)ethyl)-2,4′-bipyridin-6-amine (320 mg). LCMS (m/z): 370.0/372.0 [M+H]+; Rt=1.07 min.

### Step 2: Preparation of (R)-3,5′-dichloro-N6-(1-(tetrahydro-2H-pyran-4-yl)ethyl)-2,4′-bipyridine-2′,6-diamine

A mixture of (R)-3,5′-dichloro-2′-fluoro-N-(1-(tetrahydro-2H-pyran-4-yl)ethyl)-2,4′-bipyridin-6-amine (260 mg, 0.702 mmol) and ammonium hydroxide (aqueous solution 30-35 wt. %, 5 mL) in DMSO (5 mL) was heated in a sealed bomb at 110° C. for 18 hrs. The mixture was cooled to room temperature and was diluted with dichloromethane and water. The separated organic layer was washed with water, dried over sodium sulfate, filtered off and concentrated under reduced pressure. The residue was dissolved in acetonitrile/water and lyophilized providing crude (R)-3,5′-dichloro-N6-(1-(tetrahydro-2H-pyran-4-yl)ethyl)-2,4′-bipyridine-2′,6-diamine (240 mg), which was directly used in the next reaction without further purification. LCMS (m/z): 367.1/369.1 [M+H]+; Rt=0.95 min.

### Synthesis of 6-bromo-N-((2,2-dimethyltetrahydro-2H-pyran-4-yl)methyl)pyridin-2-amine

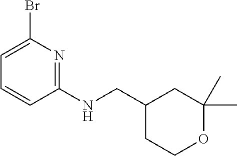

To a mixture of 6-bromopyridin-2-amine (1.2 g, 6.94 mmol) and potassium carbonate (0.479 g, 3.47 mmol) in DMF (3 mL) was added (2,2-dimethyltetrahydro-2H-pyran-4-yl)methyl 4-methylbenzenesulfonate (1.035 g, 3.47 mmol) followed by sodium hydride (60 wt. %; 0.139 g, 3.47 mmol). The mixture was stirred in a sealed tube at 40° C. for 18 hrs. The reaction mixture was diluted with EtOAc, washed with water, saturated aqueous sodium bicarbonate solution and brine. The organic layer was dried over sodium sulfate, filtered off and concentrated under reduced pressure. The crude material was purified by column chromatography [silica gel, EtOAc/hexane=0/100 to 50/50]. Fractions were combined and concentrated under reduced pressure providing 6-bromo-N-((2,2-dimethyltetrahydro-2H-pyran-4-yl)methyl)pyridin-2-amine (950 mg). LCMS (m/z): 299.0 [M+H]+; Rt=0.94 min.

### Synthesis of 5′-chloro-N6-((2,2-dimethyltetrahydro-2H-pyran-4-yl)methyl)-2,4′-bipyridine-2′,6-diamine

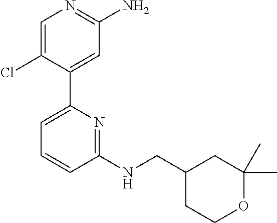

### Step 1: Preparation of (5′-chloro-2′-fluoro-[2,4′]bipyridinyl-6-yl)-(2,2-dimethyl-tetrahydro-pyran-4-ylmethyl)-carbamic acid tert-butyl ester

A mixture of tert-butyl (6-bromopyridin)-2-yl((2,2-dimethyltetrahydro-2H-pyran-4-yl)methyl)carbamate (710 mg, 1.78 mmol), 5-chloro-2-fluoropyridin-4-ylboronic acid (624 mg, 3.56 mmol), PdCl₂(dppf) CH₂Cl₂ adduct (145 mg, 0.178 mmol) in DME (7 mL) and 2M aqueous sodium carbonate solution (2.3 mL) was heated in a sealed tube at 98° C. for 2 hrs. The mixture was cooled to room temperature and was diluted with EtOAc (˜100 mL) and saturated aqueous sodium bicarbonate solution. The separated organic layer was washed with saturated aqueous sodium bicarbonate solution (2×), dried over sodium sulfate, filtered off and concentrated under reduced pressure. The residue was purified by column chromatography [silica gel, 40 g, EtOAc/heptane=0/100 to 25/75] providing (5′-chloro-2′-fluoro-[2,4′]bipyridinyl-6-yl)-(2,2-dimethyl-tetrahydro-pyran-4-ylmethyl)-carbamic acid tert-butyl ester (605 mg) as a highly viscous, colorless oil. LCMS (m/z): 394.1 {loss of tert Bu-group}/450.2 [M+H]+; Rt=1.24 min.

### Step 2: Preparation of 5′-chloro-N-((2,2-dimethyltetrahydro-2H-pyran-4-yl)methyl)-2′-fluoro-2,4′-bipyridin-6-amine

To a solution of (5′-chloro-2′-fluoro-[2,4′]bipyridinyl-6-yl)-(2,2-dimethyl-tetrahydro-pyran-4-ylmethyl)-carbamic acid tert-butyl ester (950 mg, 2.111 mmol) in MeOH (5 mL) was added 4M hydrochloride in dioxane (15 mL, 494 mmol). The mixture was stirred for ˜45 min at room temperature. The mixture was concentrated under reduced pressure and the residue was dissolved in EtOAc (˜50 mL) and saturated aqueous sodium bicarbonate solution (˜50 mL). The separated organic layer was washed with saturated aqueous sodium bicarbonate solution, brine, dried over sodium sulfate, filtered off and concentrated under reduced pressure providing crude 5′-chloro-N-((2,2-dimethyltetrahydro-2H-pyran-4-yl)methyl)-2′-fluoro-2,4′-bipyridin-6-amine (740 mg) as a colorless oil, which was directly used in the next reaction without further purification. LCMS (m/z): 350.1 [M+H]+; Rt=0.69 min.

### Step 3: Preparation of 5′-chloro-N6-((2,2-dimethyltetrahydro-2H-pyran-4-yl)methyl)-2,4′-bipyridine-2′,6-diamine

A mixture 5′-chloro-N-((2,2-dimethyltetrahydro-2H-pyran-4-yl)methyl)-2′-fluoro-2,4′-bipyridin-6-amine (370 mg, 1.058 mmol) and aqueous ammonium hydroxide solution (32 wt %, 12 mL) in DMSO (12 mL) was heated in a steel bomb at 135° C. for 16 hrs. The mixture was allowed to cool to room temperature and was diluted with EtOAc. The separated organic layer was washed with water, saturated aqueous bicarbonate solution and brine and dried over sodium sulfate, filtered off and concentrated under reduced pressure. The crude 5′-chloro-N6-(2,2-dimethyltetrahydro-2H-pyran-4-yl)methyl)-2,4′-bipyridine-2′,6-diamine (330 mg) was directly used in the next reaction without further purification. (LCMS (m/z): 347.2 [M+H]+; Rt=0.51 min.

Chiral resolution of (R)-5′-chloro-N6-((2,2-dimethyltetrahydro-2H-pyran-4-yl)methyl)-2,4′-bipyridine-2′,6-diamine and (S)-5′-chloro-N6-((2,2-dimethyltetrahydro-2H-pyran-4-yl)methyl)-2,4′-bipyridine-2′,6-diamine was accomplished as described below. Absolute stereochemistry was not determined.

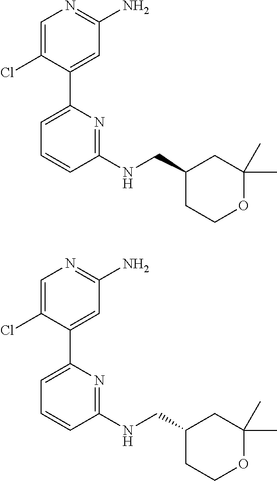

Amount: 420 mg dissolved in isopropyl alcohol, 21 mg/mL.

### Analytical Separation:

### Column: CHIRALPAK AD-H (5 um) 100×4.6 mm (Daicel Chemical Industries, LTD.).

Solvent: n-heptane: isopropyl alcohol=80:20
Flow rate: 1.0 mL/min; detection: UV=220 nm.
Fraction 1: Retention time: 6.67 min.
Fraction 2: Retention time: 12.93 min.

### Preparative Separation:

### Column: CHIRALPAK AD-prep (10 um) 2×25 cm.

Solvent: n-heptane: isopropyl alcohol=85:15
Flow rate: 20 mL/min; injection: 63 mg/3 mL; detection: UV=210 nm.
Fraction 1 (Intermediate CR1—Fraction 1): White powder. Yield: 191 mg; ee=99% (UV, 220 nm); [α]_{D}²⁰=−1.9° (c=1.0 w/v %, MeOH).
Fraction 2 (Intermediate CR1—Fraction 2): White powder. Yield: 183 mg; ee=99% (UV, 220 nm); [α]_{D}²⁰=+1.4° (c=1.0 w/v %, MeOH).

### Synthesis of tert-butyl (6-bromopyridin)-2-yl((2,2-dimethyltetrahydro-2H-pyran-4-yl)methyl)carbamate

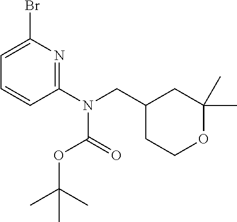

### Step 1: Preparation of tert-butyl 6-bromopyridin-2-ylcarbamate

To a solution of 6-bromopyridin-2-amine (3 g, 17.34 mmol), triethylamine (3.14 mL, 22.54 mmol) and DMAP (0.424 g, 3.47 mmol) in dichloromethane (24 mL) was slowly added a solution of BOC-anhydride (4.83 mL, 20.81 mmol) in dichloromethane (6 mL). The reaction mixture was stirred at room temperature for ˜24 hrs. The mixture was diluted with water, brine and EtOAc. The separated aqueous layer was extracted with EtOAc. The combined organic layers were dried over sodium sulfate and concentrated under reduced pressure. The residue was purified by column chromatography [silica gel] providing tert-butyl 6-bromopyridin-2-ylcarbamate (1.67 g) as a white solid. LCMS (m/z): 274.9 [M+H]+; Rt=0.95 min.

### Step 2: Preparation of (2,2-dimethyltetrahydro-2H-pyran-4-yl)methyl 4-methylbenzenesulfonate

To a solution of (2,2-dimethyltetrahydro-2H-pyran-4-yl)methanol (1 g, 6.93 mmol) in dichloromethane (5 mL) and pyridine (5 mL, 61.8 mmol) was added para-toluenesulfonyl chloride (1.586 g, 8.32 mmol) and DMAP (0.042 g, 0.347 mmol). The resulting mixture was stirred for 18 hrs at room temperature. The reaction mixture was concentrated under reduced pressure and the residue was diluted with water and dichloromethane. The separated organic phase was washed with 0.2N aqueous hydrochloride solution (1×), 1N aqueous hydrochloride solution (2×), brine, dried over sodium sulfate, filtered off and concentrated under reduced pressure. The residue was purified by column chromatography [silica gel, 40 g, EtOAc/hexane=0/100 to 50/50] providing (2,2-dimethyltetrahydro-2H-pyran-4-yl)methyl 4-methylbenzenesulfonate (2.05 g) as a colorless oil. LCMS (m/z): 299.1 [M+H]+; Rt=0.96 min.

### Step 3: Preparation of tert-butyl (6-bromopyridin)-2-yl((2,2-dimethyltetrahydro-2H-pyran-4-yl)methyl)carbamate

To a mixture of tert-butyl 6-bromopyridin-2-ylcarbamate (686 mg, 2.51 mmol), potassium carbonate (347 mg, 2.51 mmol), (2,2-dimethyltetrahydro-2H-pyran-4-yl)methyl 4-methylbenzenesulfonate (750 mg, 2.51 mmol) in DMF (10 mL) was carefully added sodium hydride (60 wt. %; 141 mg) in portions [Caution: gas development!]. The mixture was stirred at 45° C. for 4 hrs, cooled to room temperature and was diluted with EtOAc (˜50 mL) and saturated aqueous sodium bicarbonate solution. The separated organic layer was washed with saturated aqueous sodium bicarbonate solution (1×), dried over sodium sulfate, filtered off and concentrated under reduced pressure. The residue was purified by column chromatography [silica gel, 40 g, EtOAc/heptane=0/100 to 25/75] providing tert-butyl (6-bromopyridin)-2-yl((2,2-dimethyltetrahydro-2H-pyran-4-yl)methyl)carbamate (723 mg) as a highly viscous, colorless oil. LCMS (m/z): 344.9 {loss of tert Bu-group}/(399.0).[M+H]+; Rt=1.22 min.

Chiral resolution of (R)-tert-butyl (6-bromopyridin)-2-yl((2,2-dimethyltetrahydro-2H-pyran-4-yl)methyl)carbamate and (S)-tert-butyl (6-bromopyridin)-2-yl((2,2-dimethyltetrahydro-2H-pyran-4-yl)methyl)carbamate was carried out as described below. Absolute stereochemistry was not determined.

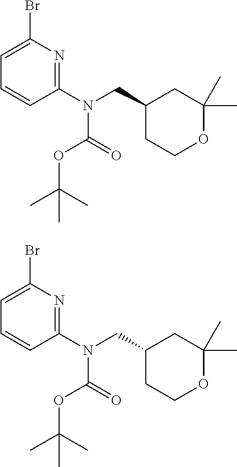

Amount: 150 g dissolved in isopropyl alcohol, 100 mg/mL.

### Analytical Separation:

### {Instrumentation: Berger SFC}

### Column: Chiralpak IC, 4.6×250 mm.

Mobile phase: CO₂/isopropyl alcohol 95:5 (isocratic).
Flow rate: 3 mL/min; BPR (back pressure): 150 bar; detection: UV=254 nm.
Injection volume: 10 μL.
Fraction 1: Retention time: 3.45 min.
Fraction 2: Retention time: 4.21 min.

### Preparative Separation:

### {Instrumentation: Thar SFC200}

### Column: Chiralpak IC, 30×250 mm.

Mobile phase: CO₂/ethanol 95:5 (isocratic) [Make-up flow: 4 mL/min CH₂Cl₂:MeOH=1:1].
Flow rate: 160 g/min; BPR 150 bar; detection: UV=280 nm.
Injection volume: 0.3 mL Cycle time 1.55 min.
Fraction 1: Nearly colorless oil. Yield: 69.74 g; ee>99.9% (UV, 254 nm); [α]_{D}²⁰=−3.3° (c=1.0 w/v %, MeOH).
Fraction 2: Nearly colorless oil. Yield: 69.31 g; ee=98.7% (UV, 254 nm); [α]_{D}²⁰=+3.4° (c=1.0 w/v %, MeOH).

### Synthesis of tert-butyl (6-bromo-5-chloropyridin)-2-yl((2,2-dimethyltetrahydro-2H-pyran-4-yl)methyl)carbamate

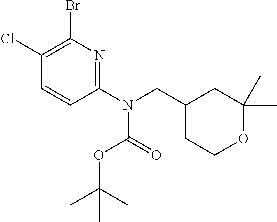

### Step 1: Preparation of tert-butyl 6-bromopyridin-2-ylcarbamate

To a solution of 6-bromo-2-aminopyridine (15 g, 87 mmol) and triethylamine (13.3 mL, 95 mmol) in dichloromethane (173 mL) was added a solution of BOC-anhydride (20.8 g, 95 mmol) in dichloromethane (100 mL) over 10 min using a syringe pump. The reaction mixture was stirred at room temperature for 72 hrs. The solvents were removed under reduced pressure and the residue was purified by column chromatography [silica gel, EtOAc/heptane=0/100 to 30/70] providing tert-butyl 6-bromopyridin-2-ylcarbamate (23.0 g) as a colorless solid. LCMS (m/z): 272.8/274.8 [M+H]+; Rt=0.97 min.

### Step 2: Preparation of tert-butyl 6-bromo-5-chloropyridin-2-ylcarbamate

To a solution of tert-butyl 6-bromopyridin-2-ylcarbamate (23.0 g, 84 mmol) in acetonitrile (281 mL) was added N-chlorosuccinimide (11.24 g, 84 mmol) and the reaction mixture was heated at 85° C. for 3 hrs. Additional N-chlorosuccinimide (5.5 g) was added and heating was continued for 3 hrs, additional N-chlorosuccinimide (5.5 g) was added and heating was continued for 1 hr. The reaction mixture was allowed to cool to room temperature and was diluted with brine (50 mL). Most of the organic solvent was removed under educed pressure and the remaining solution was extracted with EtOAc (3×). The combined organic layers were dried over sodium sulfate, filtered off and concentrated under reduced pressure. The residue was purified by column chromatography [silica gel, EtOAc/heptane=3/97] providing tert-butyl 6-bromo-5-chloropyridin-2-ylcarbamate (14.6 g) as a colorless solid. LCMS (m/z): 306.9/308.9/310.9 [M+H]+; Rt=1.14 min.

### Step 3: Preparation of (6-bromo-5-chloro-pyridin-2-yl)-(2,2-dimethyl-tetrahydro-pyran-4-ylmethyl)-carbamic acid tert-butyl ester

To a solution of tert-butyl 6-bromo-5-chloropyridin-2-ylcarbamate (2.32 g, 7.54 mmol) in DMF (25 mL) was carefully added sodium hydride (60 wt. % in mineral oil, 513 mg) and the reaction mixture was stirred at room temperature for 30 min. To reaction mixture was added a solution of (2,2-dimethyltetrahydro-2H-pyran-4-yl)methyl 4-methylbenzenesulfonate (3.15 g, 10.56 mmol) in DMF (5 mL) and stirring was continued at 25° C. for 3 hrs. The reaction mixture was partitioned between water and EtOAc. The separated organic layer was washed with water (2×), dried over sodium sulfate, filtered off and concentrated under reduced pressure. The residue was purified by column chromatography [silica gel, EtOAc/heptane=0/100 to 30/70] providing (6-bromo-5-chloro-pyridin-2-yl)-(2,2-dimethyl-tetrahydro-pyran-4-ylmethyl)-carbamic acid tert-butyl ester (2.16 g) as a colorless solid. LCMS (m/z): 432.9/434.9 [M+H]+; Rt=1.28 min.

### Synthesis of 3,5′-dichloro-N-((2,2-dimethyltetrahydro-2H-pyran-4-yl)methyl)-2′-fluoro-2,4′-bipyridin-6-amine

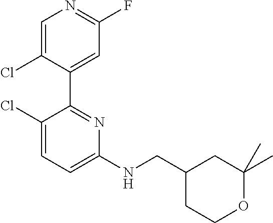

### Step 1: Preparation of (3,5′-dichloro-2′-fluoro-[2,4′]bipyridinyl-6-yl)-(2,2-dimethyl-tetrahydro-pyran-4-ylmethyl)-carbamic acid tert-butyl ester

A mixture of tert-butyl 6-bromo-5-chloropyridin-2-yl((2,2-dimethyltetrahydro-2H-pyran-4-yl)methyl)carbamate (3.08 g, 7.10 mmol), 5-chloro-2-fluoropyridin-4-ylboronic acid (2.49 g, 14.2 mmol), PdCl₂(dppf) CH₂Cl₂ adduct (0.580 g, 0.710 mmol) in DME (25.8 mL), and 2M aqueous sodium carbonate solution (8.95 mL) was heated in a sealed tube at 98° C. for 2 hrs. The reaction mixture was cooled to room temperature and was diluted with EtOAc and saturated aqueous sodium bicarbonate solution. The separated organic layer was washed with saturated aqueous sodium bicarbonate solution (2×), dried over sodium sulfate, filtered off and concentrated under reduced pressure. The residue was purified by column chromatography [silica gel, EtOAc/heptane=15/85] providing (3,5′-dichloro-2′-fluoro-[2,4′]bipyridinyl-6-yl)-(2,2-dimethyl-tetrahydro-pyran-4-ylmethyl)-carbamic acid tert-butyl ester (2.5 g) as a colorless solid. LCMS (m/z): 484.2/486.1 [M+H]+; Rt=1.33 min.

### Step 2: Preparation of 3,5′-dichloro-N-((2,2-dimethyltetrahydro-2H-pyran-4-yl)methyl)-2′-fluoro-2,4′-bipyridin-6-amine

To a mixture of (3,5′-dichloro-2′-fluoro-[2,4′]bipyridinyl-6-yl)-(2,2-dimethyl-tetrahydro-pyran-4-ylmethyl)-carbamic acid tert-butyl ester (1.20 g, 2.48 mmol) and dichloromethane (2 mL) was added trifluoroacetic acid (0.191 mL, 2.477 mmol) and the reaction mixture was stirred at room temperature for 1 hr. The reaction mixture was washed with saturated sodium bicarbonate (3×) and brine (1×). The organic layer was dried over sodium sulfate, filtered off and concentrated under educed pressure providing 3,5′-dichloro-N-((2,2-dimethyltetrahydro-2H-pyran-4-yl)methyl)-2′-fluoro-2,4′-bipyridin-6-amine as a colorless solid (940 mg). LCMS (m/z): 384.2 [M+H]+; Rt=1.07 min.

Chiral resolution of (R)-(3,5′-dichloro-2′-fluoro-[2,4′]bipyridinyl-6-yl)-(2,2-dimethyl-tetrahydro-pyran-4-ylmethyl)-carbamic acid tert-butyl ester and (S)-(3,5′-dichloro-2′-fluoro-[2,4′]bipyridinyl-6-yl)-(2,2-dimethyl-tetrahydro-pyran-4-ylmethyl)-carbamic acid tert-butyl ester was accomplished as described below. Absolute stereochemistry was not determined.

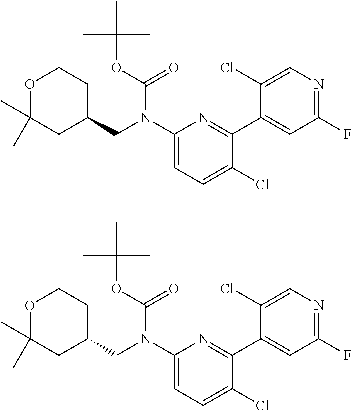

Amount: 1.65 g dissolved in isobutanol, 200 mg/mL.

### Analytical Separation:

Column: IC column (SFC).
Solvent: CO₂/isopropyl alcohol/diethylamine 95:4.9:0.1.
Flow rate: 5.0 mL/min; detection: TIC 200-400 nm.
Fraction 1: Retention time: 3.78 min.
Fraction 2: Retention time: 5.10 min.

### Preparative Separation:

### Column: CHIRALPAK AD-prep (20 um) 5×50 cm.

Solvent: n-heptane: isobutanol=98:2.
Flow rate: 40 mL/min injection: 400 mg/2 mL detection: UV=260 nm.
Fraction 1: Colorless oil. Yield: 800 mg; ee>99% (UV, 200-400 nm); [α]_{D}²⁰=+0.85° (c=1.0 w/v %, MeOH).
Fraction 2: Colorless oil. Yield: 770 mg; ee>99% (UV, 200-400 nm); [α]_{D}²⁰=−0.75° (c=1.0 w/v %, MeOH).

### Synthesis of 3,5′-dichloro-N6-((2,2-dimethyltetrahydro-2H-pyran-4-yl)methyl)-2,4′-bipyridine-2′,6-diamine

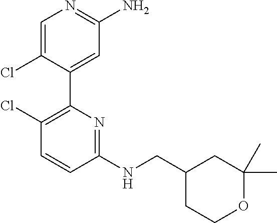

### Step 1: Preparation of 6-bromo-5-chloropyridin-2-amine

To a solution of 6-bromo-2-aminopyridine (760 mg, 4.40 mmol) in acetonitrile (15 mL) was added N-chlorosuccinimide (587 mg, 4.39 mmol) and the reaction mixture was heated at reflux for 18 hrs. The reaction mixture was cooled to 23° C. and was diluted with brine (20 mL). The mixture was concentrated under reduced pressure in order to remove most of the acetonitrile. The residue was diluted with saturated aqueous sodium carbonate solution and extracted with EtOAc (3×30 mL). The combined organic extracts were concentrated under reduced pressure and the residue was purified by column chromatography [silica gel, EtOAc/heptane=20/80 to 90/10] providing 6-bromo-5-chloropyridin-2-amine (460 mg). LCMS (m/z): 206.9, 208.9 [M+H]+; Rt=0.67 min.

### Step 2: Preparation of 6-bromo-5-chloro-N-((2,2-dimethyltetrahydro-2H-pyran-4-yl)methyl)pyridin-2-amine

To a solution of 6-bromo-5-chloropyridin-2-amine (402 mg, 1.94 mmol) in dichloromethane (5 mL) was added 2,2-dimethyltetrahydro-2H-pyran-4-carbaldehyde (276 mg, 1.94 mmol) and acetic acid (0.15 mL, 2.5 mmol). The mixture was stirred at 23° C. for 30 min and NaBH(OAc)3 (616 mg, 2.91 mmol) was added in one portion. The reaction mixture was stirred at 23° C. for additional 2 hrs. To the mixture was added brine (15 mL). The organic solvent was removed under reduced pressure and the residue was extracted with EtOAc (3×15 mL). The combined organic layers were dried over sodium sulfate, filtered off and concentrated under reduced pressure. The residue was purified by column chromatography [silica gel, EtOAc/heptane=0/100 to 40/60 ]providing 6-bromo-5-chloro-N-((2,2-dimethyltetrahydro-2H-pyran-4-yl)methyl)pyridin-2-amine (330 mg). LCMS (m/z): 332.9, 334.9 [M+H]+; Rt=1.04 min.

### Step 3: Preparation of 3,5′-dichloro-N-((2,2-dimethyltetrahydro-2H-pyran-4-yl)methyl)-2′-fluoro-2,4′-bipyridin-6-amine

A mixture of 6-bromo-5-chloro-N-((2,2-dimethyltetrahydro-2H-pyran-4-yl)methyl)pyridin-2-amine (600 mg, 1.8 mmol), 5-chloro-2-fluoropyridin-4-ylboronic acid (410 mg, 2.34 mmol) in DME (10 mL) and 2M aqueous sodium carbonate solution (3 mL) was purged with argon for 2 min and PdCl₂(dppf) CH₂Cl₂ adduct (147 mg, 0.18 mmol) was added. The mixture was heated in a sealed tube at 110° C. for 3 hrs. The mixture was allowed to cool to room temperature and the separated aqueous layer was extracted the EtOAc (3×5 mL). All organic layers were combined and concentrated under reduced pressure. The residue was purified by column chromatography [silica gel, EtOAc/heptane=0/100 to 40/60] providing 3,5′-dichloro-N-((2,2-dimethyltetrahydro-2H-pyran-4-yl)methyl)-2′-fluoro-2,4′-bipyridin-6-amine (380 mg). LCMS (m/z): 384.1 [M+H]+; Rt=1.06 min.

### Step 4: Preparation of 3,5′-dichloro-N6-((2,2-dimethyltetrahydro-2H-pyran-4-yl)methyl)-2,4′-bipyridine-2′,6-diamine

A mixture 3,5′-dichloro-N-((2,2-dimethyltetrahydro-2H-pyran-4-yl)methyl)-2′-fluoro-2,4′-bipyridin-6-amine (360 mg, 0.94 mmol) and aqueous ammonium hydroxide solution (30-35 wt. %, 7 mL) in DMSO (7 mL) was heated in a steel bomb at 130° C. for 20 hrs. The mixture was allowed to cool to room temperature and was diluted with EtOAc (20 mL). The organic layer was washed with brine and concentrated under reduced pressure. The residue was purified by column chromatography [silica gel, EtOAc/heptane=10/90 to 70/30]. Fractions were combined and concentrated under reduced pressure providing 3,5′-dichloro-N6-((2,2-dimethyltetrahydro-2H-pyran-4-yl)methyl)-2,4′-bipyridine-2′,6-diamine (290 mg). (LCMS (m/z): 381.1, 383.0 [M+H]+; Rt=0.68 min.

### Synthesis of 6-(((2,2-dimethyltetrahydro-2H-pyran-4-yl)methyl)amino)-5-fluoropyridin-2-yl trifluoromethanesulfonate

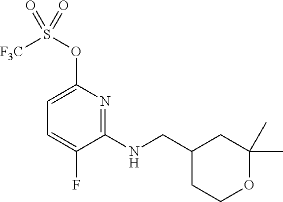

### Step 1: Preparation of (2,2-dimethyltetrahydro-2H-pyran-4-yl)methanamine

Into a solution of (2,2-dimethyltetrahydro-2H-pyran-4-yl)methyl 4-methylbenzenesulfonate (3 g, 10.05 mmol) in tetrahydrofuran (25 mL) in a steel bomb was condensed ammonia (˜5.00 mL) at −78° C. The mixture was heated in the steel bomb at 125° C. for ˜18 hrs. The mixture was cooled to −78° C., the steel bomb was opened, and the mixture was allowed to warm up to room temperature under a stream of nitrogen. The mixture was concentrated under reduced pressure and the residue was partitioned between a aqueous sodium hydroxide solution (5 wt. %) and dichloromethane. The separated aqueous layer was extracted with dichloromethane (1×). The combined organic layers were washed with aqueous sodium hydroxide solution (5 wt. %), dried over sodium sulfate, filtered off and concentrated under reduced pressure providing crude (2,2-dimethyltetrahydro-2H-pyran-4-yl)methanamine (˜2.36 g) as yellow liquid, which was directly used in the next reaction without further purification. LCMS (m/z): 144.1 [M+H]+; Rt=0.26 min.

### Step 2: Preparation of N-((2,2-dimethyltetrahydro-2H-pyran-4-yl)methyl)-3,6-difluoropyridin-2-amine

A mixture of 2,3,6-trifluoropyridine (1.827 g, 13.73 mmol), crude (2,2-dimethyltetrahydro-2H-pyran-4-yl)methanamine (2.36 g, 16.48 mmol) and triethylamine (4.59 mL, 33.0 mmol) in NMP (40 mL) was heated at 70° C. for 1 hr. The reaction mixture was cooled room temperature and was diluted with EtOAc (˜100 mL), brine (˜50 mL) and water (˜50 mL). The separated organic layer was washed with brine (1×), 0.3N aqueous hydrochloride solution (2×), saturated aqueous sodium bicarbonate solution (1×), brine (1×), dried over sodium sulfate, filtered and concentrated under reduced pressure. The residue was purified by column chromatography [silica gel, 40 g, EtOAc/heptane=0/100 to 30/70] providing N-((2,2-dimethyltetrahydro-2H-pyran-4-yl)methyl)-3,6-difluoropyridin-2-amine (1.96 g) as a colorless oil. LCMS (m/z): 257.0 [M+H]+; Rt=0.96 min.

### Step 3: Preparation of N-((2,2-dimethyltetrahydro-2H-pyran-4-yl)methyl)-3-fluoro-6-methoxypyridin-2-amine

To a solution of N-((2,2-dimethyltetrahydro-2H-pyran-4-yl)methyl)-3,6-difluoropyridin-2-amine (1.90 g, 7.41 mmol) in MeOH (15 mL) was added sodium methoxide (25 wt. %; 5.09 mL) The mixture was heated in a steel bomb at 135° C. for ˜18 hrs. Additional sodium methoxide (25 wt. %; 1.695 mL) was added and heating was continued for ˜24 hrs. The mixture was cooled to room temperature and was diluted with brine and EtOAc. To the separated aqueous layer was added 1N aqueous hydrochloride solution and EtOAc. The separated aqueous layer was neutralized with saturated aqueous sodium bicarbonate solution and diluted with EtOAc. The combined organic layers were washed with brine, dried over sodium sulfate, filtered off and concentrated under reduced pressure providing crude N-((2,2-dimethyltetrahydro-2H-pyran-4-yl)methyl)-3-fluoro-6-methoxypyridin-2-amine (˜2.14 g) as a brownish liquid, which was directly used in the next reaction without further purification. LCMS (m/z): 269.3 [M+H]+; Rt=0.96 min.

### Step 4: Preparation of 6-(((2,2-dimethyltetrahydro-2H-pyran-4-yl)methyl)amino)-5-fluoropyridin-2-ol

To N-((2,2-dimethyltetrahydro-2H-pyran-4-yl)methyl)-3-fluoro-6-methoxypyridin-2-amine (2.135 g, 7.96 mmol) in acetonitrile (20 mL) was added sodium iodide (8.35 g, 55.7 mmol) and chlorotrimethylsilane (7.12 mL, 55.7 mmol). The mixture was heated to reflux (oil bath: 93° C.) for 5 hrs. The mixture was allowed to cool to room temperature and was diluted with EtOAc and saturated aqueous sodium bicarbonate solution and vigorously stirred for 15 min. The mixture was acidified with 0.5N aqueous hydrochloride solution and stirring was continued for 5 min. The mixture was neutralized with saturated aqueous sodium bicarbonate solution. The separated aqueous phase was extracted with EtOAc (3×). The combined organic layers were dried over sodium sulfate, filtered off and concentrated under reduced pressure. The residue was purified by column chromatography [silica gel, 80 g, EtOAc/heptane=5/95 to 50/50] providing 6-(((2,2-dimethyltetrahydro-2H-pyran-4-yl)methyl)amino)-5-fluoropyridin-2-ol (245 mg) as a colorless, highly viscous oil. LCMS (m/z): 255.1 [M+H]+; Rt=0.56 min.

### Alternative preparation of 6-(((2,2-dimethyltetrahydro-2H-pyran-4-yl)methyl)amino)-5-fluoropyridin-2-ol

### Step A-3: Preparation of N-((2,2-dimethyltetrahydro-2H-pyran-4-yl)methyl)-3-fluoro-6-(4-methoxybenzyloxy)pyridin-2-amine

To 4-methoxybenzyl alcohol (10.67 g, 77 mmol) was added potassium tert-butoxide (1M solution in tert-butanol, 77 mL) and the mixture was stirred at room temperature for 30 min to yield a dark yellow solution, to which was slowly added a solution of N-((2,2-dimethyltetrahydro-2H-pyran-4-yl)methyl)-3,6-difluoropyridin-2-amine (6.6 g, 25.8 mmol) in tetrahydrofuran (50 mL). The resulting orange mixture was heated at 90° C. for 24 hrs. The reaction mixture cooled to room temperature and was poured into water and extracted with EtOAc. The combined organic extracts were washed with brine, dried with sodium sulfate and concentrated under reduced pressure. The residue was filtered (2×) using column chromatography [silica gel, 120 g, EtOAc/heptane=0/100 to 15/85] providing N-((2,2-dimethyltetrahydro-2H-pyran-4-yl)methyl)-3-fluoro-6-(4-methoxybenzyloxy)pyridin-2-amine (7.8 g; purity ˜50% by LCMS) as a light yellow solid, which was used directly in the next step without further purification. LCMS (m/z): 375 [M+H]+; Rt=1.12 min.

### Step A-4: Preparation of 6-(((2,2-dimethyltetrahydro-2H-pyran-4-yl)methyl)amino)-5-fluoropyridin-2-ol

A solution of N-((2,2-dimethyltetrahydro-2H-pyran-4-yl)methyl)-3-fluoro-6-(4-methoxybenzyloxy)pyridin-2-amine (7.8 g, 20.83 mmol) in EtOH (250 mL) was degassed with argon and Pd/C (10 wt. %; 1.108 g) was added. The mixture was stirred under hydrogen atmosphere (˜1 atm, balloon) for 16 hrs. The reaction mixture was filtered through celites and rinsed with dichloromethane. The filtrate was concentrated under reduced pressure. The residue was purified by column chromatography [silica gel, 120 g, EtOAc/heptane=0/100 to 35/65]. Fractions were combined and concentrated under reduced pressure providing 6-(((2,2-dimethyltetrahydro-2H-pyran-4-yl)methyl)amino)-5-fluoropyridin-2-ol (3.5 g) as a violet oil. LCMS (m/z): 255.0 [M+H]+; Rt=0.53 min.

### Step 5: Preparation of 6-(((2,2-dimethyltetrahydro-2H-pyran-4-yl)methyl)amino)-5-fluoropyridin-2-yl trifluoromethanesulfonate

To a solution of 6-((2,2-dimethyltetrahydro-2H-pyran-4-yl)methylamino)-5-fluoropyridin-2-ol (245 mg, 0.963 mmol) and triethylamine (0.403 mL, 2.89 mmol) in dichloromethane (12 mL) was added trifluoromethanesulfonic anhydride (0.244 mL, 1.445 mmol) slowly at 0° C. The mixture was stirred for 2 hrs at 0° C. and poured carefully into ice-cooled saturated aqueous sodium bicarbonate solution. The separated aqueous layer was extracted with dichloromethane (2×). The combined organic layers were dried over sodium sulfate, filtered off and concentrated under reduced pressure. The residue was purified by column chromatography [silica gel, 24 g, 20 min, EtOAc/heptane=5/95 to 40/60]. Pure fractions were combined and concentrated under reduced pressure providing 6-(((2,2-dimethyltetrahydro-2H-pyran-4-yl)methyl)amino)-5-fluoropyridin-2-yl trifluoromethanesulfonate (200 mg) as a colorless oil. LCMS (m/z): 387.2 [M+H]+; Rt=1.09 min.

### Synthesis of 5′-chloro-N6-((2,2-dimethyltetrahydro-2H-pyran-4-yl)methyl)-5-fluoro-2,4′-bipyridine-2′,6-diamine

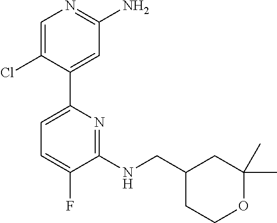

### Step 1: Preparation of 5′-chloro-N-((2,2-dimethyltetrahydro-2H-pyran-4-yl)methyl)-2′,5-difluoro-2,4′-bipyridin-6-amine

A mixture of 6-((2,2-dimethyltetrahydro-2H-pyran-4-yl)methylamino)-5-fluoropyridin-2-yl trifluoromethanesulfonate (200 mg, 0.518 mmol), 5-chloro-2-fluoropyridin-4-ylboronic acid (182 mg, 1.035 mmol), PdCl₂(dppf) CH₂Cl₂ adduct (42.3 mg, 0.052 mmol) in DME (2.4 mL) and 2M sodium carbonate solution (0.8 mL, 1.60 mmol) in a sealed tube was heated at 95° C. for 3 hrs. The mixture was cooled to room temperature and was diluted with EtOAc (˜100 mL) and saturated aqueous sodium bicarbonate carbonate solution. The separated organic layer was washed with saturated aqueous sodium bicarbonate carbonate solution (2×), dried over sodium sulfate, filtered off and concentrated under reduced pressure. The residue was purified by column chromatography [silica gel, 24 g, EtOAc/heptane=0/100 to 25/75] providing 5′-chloro-N-((2,2-dimethyltetrahydro-2H-pyran-4-yl)methyl)-2′,5-difluoro-2,4′-bipyridin-6-amine (135 mg) as a white solid. Fractions were combined and concentrated under reduced pressure. LCMS (m/z): 368.2 [M+H]+; Rt=1.08 min.

### Step 2: Preparation of 5′-chloro-N6-((2,2-dimethyltetrahydro-2H-pyran-4-yl)methyl)-5-fluoro-2,4′-bipyridine-2′,6-diamine

A mixture of 5′-chloro-N-((2,2-dimethyltetrahydro-2H-pyran-4-yl)methyl)-2′,5-difluoro-2,4′-bipyridin-6-amine (135 mg, 0.367 mmol) and ammonium hydroxide (aqueous solution 30-35 wt. %, 6 mL) in DMSO (4 mL) was heated in a steel bomb at 140° C. for 24 hrs. The mixture was cooled to room temperature and was diluted with water and EtOAc. The separated organic layer was washed with saturated aqueous sodium bicarbonate solution (2×), dried over sodium sulfate, filtered off and concentrated under reduced pressure providing crude 5′-chloro-N6-((2,2-dimethyltetrahydro-2H-pyran-4-yl)methyl)-5-fluoro-2,4′-bipyridine-2′,6-diamine (133 mg), which was directly used in the next reaction without further purification. LCMS (m/z): 365.1 [M+H]+; Rt=0.68 min.

### Synthesis of 6-bromo-N-((6,6-dimethyl-1,4-dioxan-2-yl)methyl)pyridin-2-amine

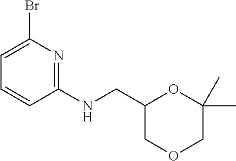

### Step 1: Preparation of 1-(allyloxy)-2-methylpropan-2-ol

To allylic alcohol (57.4 mL, 844 mmol) was added sodium hydride (60 wt. % in mineral oil, 2.43 g, 101 mmol) at 0° C. After stirring for 20 min 2,2-dimethyloxirane (15 mL, 169 mmol) was added and the solution was refluxed overnight. The mixture was allowed to cool to room temperature, diluted with saturated aqueous ammonium chloride solution and extracted with diethylether (3×). The combined organic layers were dried over sodium sulfate, filtered off and concentrated under reduced pressure to remove diethylether. The residue was distilled providing 1-(allyloxy)-2-methylpropan-2-ol (12.3 g, 42 torr, by 58-60° C.) as a colorless oil. ¹H NMR (400 MHz, chloroform-d) δ [ppm]: 5.87-5.96 (m, 1H), 5.26-5.31 (m, 1H), 5.18-5.21 (m, 1H), 4.03-4.05 (m, 2H), 3.28 (s, 2H), 2.31 (br. s, 1H), 1.23, (s, 3H), 1.22 (s, 3H).

### Step 2: Preparation of 2-methyl-1-(oxiran-2-ylmethoxy)propan-2-ol

To a solution of 1-(allyloxy)-2-methylpropan-2-ol (1.50 g, 11.5 mmol) in dichloromethane (50 mL) was added MCPBA (<77 wt. %, 9.94 g) at 0° C. The suspension was stirred at 0° C. for 6.5 hrs before saturated aqueous sodium bicarbonate solution and aqueous sodium thiosulfate solution were added. The mixture was stirred at 0° C. for 15 min. The separated aqueous layer was extracted with dichloromethane (2×). The combined organic layers were dried over sodium sulfate, filtered off and concentrated under reduced pressure. The residue was purified by column chromatography [silica gel, EtOAc/heptane=0/100 to 67/33] providing 2-methyl-1-(oxiran-2-ylmethoxy)propan-2-ol as a colorless oil (620 mg). ¹H NMR (400 MHz, chloroform-d) δ [ppm]: 3.64 (ddd, J=12.0, 5.2, 2.8 Hz, 1H), 3.24-3.29 (m, 1H), 3.17-3.21 (m, 1H), 3.11-3.14 (m, 1H), 2.97-3.00 (m, 1H), 2.88 (br. s, 1H), 2.60-2.64 (m, 1 H), 2.44-2.47 (m, 1H), 1.02 (s, 6H).

### Step 3: Preparation of (6,6-dimethyl-1,4-dioxan-2-yl)methanol

A solution of 2-methyl-1-(oxiran-2-ylmethoxy)propan-2-ol (620 mg, 4.24 mmol) and (±)-camphor-10-sulfonic acid (300 mg, 1.29 mmol) in dichloromethane (30 mL) was stirred at room temperature for 24 hrs. The mixture was diluted with saturated aqueous sodium bicarbonate solution. The separated aqueous phase was extracted with dichloromethane (4×). The organic layers were combined, dried over sodium sulfate, filtered off and concentrated under reduced pressure. The residue was purified by column chromatography [silica gel, EtOAc/heptane=0/100 to 67/33] providing (6,6-dimethyl-1,4-dioxan-2-yl)methanol (400 mg) as a colorless oil. ¹H NMR (400 MHz, chloroform-d) δ [ppm]: 3.90-3.96 (m, 1H), 3.76 (dd, J=11.2, 2.8 Hz, 1H), 3.56 (dd, J=11.6, 4.0 Hz, 1H), 3.46-3.50 (m, 2H), 3.29 (t, J=11.2 Hz, 1H), 3.24 (dd, J=11.6, 1.2 Hz, 1H), 2.69 (br. s, 1H), 1.35 (s, 3H), 1.13 (s, 3H).

### Step 4: Preparation of (6,6-dimethyl-1,4-dioxan-2-yl)methyl methanesulfonate

To a solution of triethylamine (0.52 mL, 3.74 mmol) and (6,6-dimethyl-1,4-dioxan-2-yl)methanol (390 mg, 2.67 mmol) in dichloromethane (10 mL) was slowly added methanesulfonyl chloride (0.249 mL, 3.20 mmol) at 0° C. After the addition was completed the solution was warmed to room temperature and stirred for 1 hr. The mixture was diluted with saturated aqueous sodium bicarbonate solution. The separated aqueous layer was extracted with dichloromethane (3×). The combined organic layers were dried over sodium sulfate, filtered off and concentrated under reduced pressure. The residue was purified by column chromatography [silica gel, EtOAc/heptane=20/80 to 50/50] providing (6,6-dimethyl-1,4-dioxan-2-yl)methyl methanesulfonate (584 mg) as a colorless oil. ¹H NMR (400 MHz, chloroform-d) δ [ppm]: 4.00-4.09 (m, 3H), 3.74 (dd, J=11.2, 2.8 Hz, 1H), 3.42 (d, J=11.6 Hz, 1H), 3.16-3.23 (m, 2H), 2.99 (s, 3H), 1.27 (s, 3H), 1.05 (s, 3H).

### Step 5: Preparation of 6-bromo-N-((6,6-dimethyl-1,4-dioxan-2-yl)methyl)pyridin-2-amine

To a solution of 6-bromopyridin-2-amine (722 mg, 4.17 mmol) in anhydrous DMF (8 mL) was added sodium hydride (60 wt. % in mineral oil, 195 mg) at 0° C. After stirring for 10 min the solution was warmed up to room temperature and stirred for additional 45 min. The solution was cooled to 0° C. and a solution of (6,6-dimethyl-1,4-dioxan-2-yl)methyl methanesulfonate (520 mg, 2.32 mmol) in DMF (2 mL) was added. After the addition was completed the mixture was warmed to room temperature and stirred overnight. The mixture was diluted with EtOAc and washed with water (4×). The combined aqueous layers were extracted with EtOAc (1×). The combined organic layers were dried over sodium sulfate, filtered off and concentrated under reduced pressure. The residue was purified by hplc. Fractions were combined, concentrated under reduced pressure, basified with sodium carbonate and extracted with EtOAc (3×). The combined organic layers were dried over sodium sulfate, filtered off and concentrated under reduced pressure providing 6-bromo-N-((6,6-dimethyl-1,4-dioxan-2-yl)methyl)pyridin-2-amine as a light yellow oil (270 mg). LCMS (m/z): 301.0/303.0 [M+H]+; Rt=0.86 min.

### Synthesis of 5′-chloro-N6-((6,6-dimethyl-1,4-dioxan-2-yl)methyl)-2,4′-bipyridine-2′,6-diamine

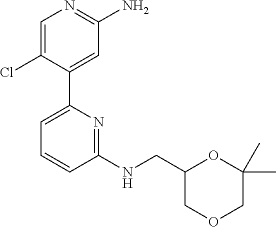

### Step 1: Preparation of 5′-chloro-N-((6,6-dimethyl-1,4-dioxan-2-yl)methyl)-2′-fluoro-2,4′-bipyridin-6-amine

A mixture of 6-bromo-N-((6,6-dimethyl-1,4-dioxan-2-yl)methyl)pyridin-2-amine (260 mg, 0.863 mmol), 5-chloro-2-fluoropyridin-4-ylboronic acid (303 mg, 1.73 mmol), PdCl₂(dppf) CH₂Cl₂ adduct (70.5 mg, 0.086 mmol) and sodium carbonate (274 mg, 2.59 mmol) in DME (4 mL) and water (2 mL) was sonicated and heated in a sealed tube at 110° C. for 20 min in a microwave reactor. The mixture was diluted with water and extracted with EtOAc (3×). The combined organic layers were dried over sodium sulfate and concentrated under reduced pressure. The residue was purified by column chromatography [silica gel, EtOAc/dichloromethane=1/10 to 1/4] providing 5′-chloro-N-((6,6-dimethyl-1,4-dioxan-2-yl)methyl)-2′-fluoro-2,4′-bipyridin-6-amine as a colorless oil (245 mg). LCMS (m/z): 352.1 [M+H]+; Rt=0.68 min.

### Step 2: Preparation of 5′-chloro-N6-((6,6-dimethyl-1,4-dioxan-2-yl)methyl)-2,4′-bipyridine-2′,6-diamine

A mixture of 5′-chloro-N-((6,6-dimethyl-1,4-dioxan-2-yl)methyl)-2′-fluoro-2,4′-bipyridin-6-amine (185 mg, 0.526 mmol) and ammonium hydroxide (aqueous solution 30-35 wt. %, 1.5 mL) in DMSO (1 mL) was heated in a steel bomb at 130° C. for ˜16 hrs. The mixture was cooled to room temperature and was diluted with EtOAc. The mixture was washed with water (4×) and the combined aqueous layers were extracted with EtOAc. The combined organic layers were dried over sodium sulfate, filtered off and concentrated under reduced pressure. The residue was purified by column chromatography [silica gel, EtOAc/dichloromethane=33/67 to 100/0]. Fractions were combined and concentrated under reduced pressure providing 5′-chloro-N-((6,6-dimethyl-1,4-dioxan-2-yl)methyl)-2′-fluoro-2,4′-bipyridin-6-amine (68 mg). LCMS (m/z): 349.1 [M+H]+; Rt=0.50 min.

### Synthesis of 6-bromo-N-((5,5-dimethyl-1,4-dioxan-2-yl)methyl)pyridin-2-amine

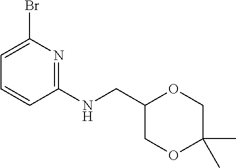

### Step 1: Preparation of 2-(allyloxy)-2-methylpropan-1-ol

To a solution of 2,2-dimethyloxirane (15.0 mL, 169 mmol) in allylic alcohol (57.4 mL) was added perchloric acid (70 wt. %, 7.26 mL, 84 mmol) slowly at 0° C. The solution was warmed to room temperature and stirred for 1.5 hrs. The reaction mixture was diluted with saturated aqueous sodium bicarbonate solution and extracted with diethylether (3×). The combined organic layers were dried over sodium sulfate, filtered off and concentrated under reduced pressure to remove diethylether. The residue was distilled providing 2-(allyloxy)-2-methylpropan-1-ol (9.70 g, 38 torr, by 74-76° C.) as a colorless oil. ¹H NMR (400 MHz, chloroform-d) δ [ppm]: 5.87-5.97 (m, 1H), 5.25-5.31 (m, 1H), 5.12-5.16 (m, 1H), 3.92-3.94 (m, 2H), 3.45 (m, 2H), 1.19 (s, 6H).

### Step 2: Preparation of 2-methyl-2-(oxiran-2-ylmethoxy)propan-1-ol

To a solution of 2-(allyloxy)-2-methylpropan-1-ol (2.37 g, 18.2 mmol) in dichloromethane (70 mL) was added MCPBA (<77 wt. %, 15.71 g) at 0° C. The suspension was stirred at 0° C. for 6.5 hrs before saturated aqueous sodium bicarbonate solution and aqueous sodium thiosulfate solution were added. The mixture was stirred at 0° C. for 15 min. The separated aqueous layer was extracted with dichloromethane (2×). The combined organic layers were dried over sodium sulfate, filtered off and concentrated under reduced pressure. The residue was purified by column chromatography [silica gel, EtOAc/heptane=0/100 to 67/33] providing 2-methyl-2-(oxiran-2-ylmethoxy)propan-1-ol as a colorless oil (910 mg). ¹H NMR (400 MHz, chloroform-d) δ [ppm]: 3.65 (dd, J=11.2, 2.8 Hz, 1H), 3.47 (br. s, 1H), 3.31-3.41 (m, 3H), 3.07-3.09 (m, 1H), 2.74 (t, J=4.8 Hz, 1H), 2.63-2.65 (m, 1H), 1.12 (s, 6H).

### Step 3 Preparation of (5,5-dimethyl-1,4-dioxan-2-yl)methanol

A solution of 2-methyl-2-(oxiran-2-ylmethoxy)propan-1-ol (870 mg, 5.95 mmol) and (±)-camphor-10-sulfonic acid (207 mg) in dichloromethane (70 mL) was stirred at room temperature for 24 hrs. Additional (±)-camphor-10-sulfonic acid (100 mg) was added and stirring was continued overnight. The mixture was diluted with saturated aqueous sodium bicarbonate solution. The separated aqueous phase was extracted with dichloromethane (2×). The combined organic layers were dried over sodium sulfate, filtered off and concentrated under reduced pressure providing crude (5,5-dimethyl-1,4-dioxan-2-yl)methanol as a colorless oil (750 mg), which was directly used in the next step without further purification. ¹H NMR (400 MHz, chloroform-d) δ [ppm]: 3.69-3.74 (m, 1H), 3.52-3.64 (m, 5H), 3.43 (dd, J=11.6, 0.8 Hz, 1H), 2.57 (br. s, 1H), 1.32 (s, 3H), 1.13 (s, 3H).

### Step 4: Preparation of (5,5-dimethyl-1,4-dioxan-2-yl)methyl methanesulfonate

To a solution of triethylamine (0.988 mL, 7.09 mmol) and (5,5-dimethyl-1,4-dioxan-2-yl)methanol (740 mg, 5.06 mmol) in dichloromethane (20 mL) was slowly added methanesulfonyl chloride (0.473 mL, 6.07 mmol) at 0° C. After the addition was completed the solution was warmed to room temperature and stirred for 1 hr. The mixture was diluted with saturated aqueous sodium bicarbonate solution. The separated aqueous layer was extracted with dichloromethane (3×). The combined organic layers were dried over sodium sulfate, filtered off and concentrated under reduced pressure. The residue was purified by column chromatography [silica gel, EtOAc/heptane=20/80 to 50/50] providing (5,5-dimethyl-1,4-dioxan-2-yl)methyl methanesulfonate (805 mg) as a colorless oil. ¹H NMR (400 MHz, chloroform-d) δ [ppm]: 4.18-4.19 (m, 2H), 3.71-3.76 (m, 1H), 3.66 (t, J=10.8 Hz, 1H), 3.52-3.57 (m, 2H), 3.37 (d, J=11.6 Hz, 1H), 3.03 (s, 3H), 1.28 (s, 3H), 1.09 (s, 3H).

### Step 5: Preparation of 6-bromo-N-((5,5-dimethyl-1,4-dioxan-2-yl)methyl)pyridin-2-amine

To a solution of 6-bromopyridin-2-amine (771 mg, 4.46 mmol) in anhydrous DMF (10 mL) was added sodium hydride (60 wt. % in mineral oil, 214 mg, 5.35 mmol) at 0° C. After 10 min the solution was warmed up to room temperature and stirred for additional 15 min. A solution of (5,5-dimethyl-1,4-dioxan-2-yl)methyl methanesulfonate (500 mg, 2.23 mmol) in DMF (2 mL) was added at 0° C. After the addition was completed the mixture was warmed to room temperature and stirred for 20 min and at 60° C. for 1.5 hrs. The mixture was cooled to room temperature, diluted with EtOAc and washed with water (4×). The combined aqueous layers were extracted with EtOAc (1×). The combined organic layers were dried over sodium sulfate, filtered off and concentrated under reduced pressure. The residue was purified by column chromatography [silica gel, EtOAc/heptane=0/100 to 50/50], followed by [silica gel, dichloromethane/diethylether=20/1] providing 6-bromo-N-((5,5-dimethyl-1,4-dioxan-2-yl)methyl)pyridin-2-amine (306 mg). LCMS (m/z): 301.0/303.0 [M+H]+; Rt=0.89 min.

### Synthesis of 5′-chloro-N6-((5,5-dimethyl-1,4-dioxan-2-yl)methyl)-2,4′-bipyridine-2′,6-diamine

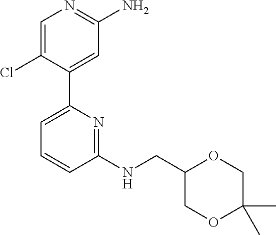

### Step 1: Preparation of 5′-chloro-N-((5,5-dimethyl-1,4-dioxan-2-yl)methyl)-2′-fluoro-2,4′-bipyridin-6-amine

A mixture of 6-bromo-N-((5,5-dimethyl-1,4-dioxan-2-yl)methyl)pyridin-2-amine (294 mg, 0.976 mmol), 5-chloro-2-fluoropyridin-4-ylboronic acid (256 mg, 1.46 mmol), PdCl₂(dppf) CH₂Cl₂ adduct (80 mg, 0.097 mmol) and sodium carbonate (310 mg, 2.93 mmol) in DME (4 mL) and water (2 mL) was sonicated and heated in a sealed tube at 100° C. for 20 min in a microwave reactor. Additional 5-chloro-2-fluoropyridin-4-ylboronic acid (34 mg, 0.19 mmol) and PdCl₂(dppf) CH₂Cl₂ adduct (16 mg, 0.019 mmol) were added and heating was continued at 110° C. for 10 min in the reactor. The mixture was diluted with water and extracted with EtOAc (3×). The combined organic layers were dried over sodium sulfate and concentrated under reduced pressure The residue was purified by column chromatography [silica gel, EtOAc/heptane=0/100 to 33/67] providing 5′-chloro-N-((5,5-dimethyl-1,4-dioxan-2-yl)methyl)-2′-fluoro-2,4′-bipyridin-6-amine as a light yellow oil (241 mg). LCMS (m/z): 352.1 [M+H]+; Rt=0.69 min.

### Step 2: Preparation of 5′-chloro-N6-((5,5-dimethyl-1,4-dioxan-2-yl)methyl)-2,4′-bipyridine-2′,6-diamine

A mixture of 5′-Chloro-N-((5,5-dimethyl-1,4-dioxan-2-yl)methyl)-2′-fluoro-2,4′-bipyridin-6-amine (165 mg, 0.469 mmol) and ammonium hydroxide (aqueous solution 30-35 wt. %, 1.5 mL) in DMSO (1.5 mL) was heated in a steel bomb at 130° C. for ˜16 hrs. The mixture was cooled to room temperature and was diluted with EtOAc. The mixture was washed with water (4×) and the combined aqueous layers were extracted with EtOAc. The combined organic layers were dried over sodium sulfate, filtered off and concentrated under reduced pressure. The residue was purified by column chromatography [silica gel, EtOAc/dichloromethane=33/67 to 100/0]. Fractions were combined and concentrated under reduced pressure providing 5′-chloro-N6-((5,5-dimethyl-1,4-dioxan-2-yl)methyl)-2,4′-bipyridine-2′,6-diamine (136 mg). (LCMS (m/z): 349.2 [M+H]+; Rt=0.49 min.

### Synthesis of 6-bromo-N-(((2R,6S)-2,6-dimethyltetrahydro-2H-pyran-4-yl)methyl)pyridin-2-amine

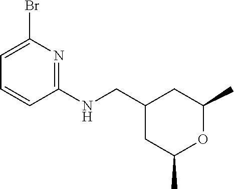

### Step 1: Preparation of (2R,6S)-2,6-dimethyldihydro-2H-pyran-4(3H)-one

A solution of 2,6-dimethyl-4H-pyran-4-one (2 g, 16.1 mmol) in EtOH (20 mL) was stirred over Pd/C (10 wt. %, 0.2 g) under hydrogen (15 psi) for 16 hrs at ambient temperature. The suspension was filtered off and the filtrate was concentrated under reduced pressure. The residue was dissolved in dichloromethane (15 mL) and treated with Dess-Martin periodinane (2.3 g) at ambient temperature for 16 hrs. To the suspension was added saturated aqueous sodium thiosulfate solution (˜3 mL) and the mixture was stirred for 1 hr. The mixture was diluted with saturated aqueous sodium bicarbonate solution (20 mL) and stirred for an additional 1 hr. The separated organic phase was washed with water and brine, dried over sodium sulfate, filtered through celite and concentrated under reduced pressure. The residue was purified by column chromatography [silica gel, EtOAc/heptane=10/90]. Fractions were combined and concentrated under reduced pressure providing (2R,6S)-2,6-dimethyldihydro-2H-pyran-4(3H)-one (600 mg). GCMS: 128 [M]; Rt=4.25 min. ¹H NMR (400 MHz, DMSO-d6) δ [ppm]: 1.18 (d, J=6.26 Hz, 6H) 2.11-2.25 (m, 4H) 3.58-3.77 (m, 2H).

### Step 2: Preparation of (2R,6S)-4-(methoxymethylene)-2,6-dimethyltetrahydro-2H-pyran

To a suspension of (methoxymethyl)triphenyl phosphine chloride (1.5 g, 4.45 mmol) in tetrahydrofuran (8 mL) was added slowly sodium bis(trimethylsilyl) amide (1M solution in tetrahydrofuran, 4.45 mL) at −10° C. The reaction mixture was stirred for 1 hr and a solution of (2R,6S)-2,6-dimethyldihydro-2H-pyran-4(3H)-one (380 mg, 2.96 mmol) in tetrahydrofuran (2 mL) was added slowly. The resulting mixture was allowed to warm to ambient temperature and stirred for 3 hrs. The reaction mixture was diluted with water (15 mL) and extracted with diethylether (2×30 mL). The combined organic layers were washed with brine, dried over sodium sulfate, filtered off and concentrated under reduced pressure. The residue was purified by column chromatography [silica gel, EtOAc/heptane=10/90] providing (2R,6S)-4-(methoxymethylene)-2,6-dimethyltetrahydro-2H-pyran (240 mg) as a colorless oil. GCMS: 156 [M]; Rt=5.40 min. ¹H NMR (400 MHz, DMSO-d6) δ [ppm]: 1.07 (t, J=6.06 Hz, 6H) 1.18-1.29 (m, 1H) 1.31-1.46 (m, 1H) 1.61 (t, J=12.13 Hz, 1H) 1.93 (d, J=13.30 Hz, 1H) 3.17-3.28 (m, 2H) 3.46 (s, 3H) 5.89 (s, 1H).

### Step 3: Preparation of (2R,6S)-2,6-dimethyltetrahydro-2H-pyran-4-carbaldehyde

A mixture of (2R,6S)-4-(methoxymethylene)-2,6-dimethyltetrahydro-2H-pyran (240 mg, 1.53 mmol) and formic acid (˜88 wt. % in water, 1.5 mL, 34.4 mmol) under argon was heated at 90° C. for 1 hr. The reaction mixture was cooled to 0° C., neutralized with 1N aqueous sodium hydroxide solution until pH˜6 and extracted with diethylether. The organic layer were dried over sodium sulfate, filtered off and concentrated under reduced pressure providing crude (2R,6S)-2,6-dimethyltetrahydro-2H-pyran-4-carbaldehyde (120 mg) as a yellow oil, which was directly used in the next reaction without further purification. GCMS: 142 [M]; Rt=5.0 min. ¹H NMR (400 MHz, DMSO-d6) δ [ppm]: 0.89-1.00 (m, 2H) 1.09 (d, J=6.26 Hz, 6H) 1.77 (ddd, J=12.33, 1.96, 1.76 Hz, 2H) 3.35 (t, J=7.04 Hz, 1H) 3.38-3.48 (m, 2H) 9.51 (s, 1H).

### Step 4: Preparation of 6-bromo-N-(((2R,6S)-2,6-dimethyltetrahydro-2H-pyran-4-yl)methyl)pyridin-2-amine

A mixture of (2R,6S)-2,6-dimethyltetrahydro-2H-pyran-4-carbaldehyde (120 mg, 0.84 mmol) and 6-bromo-2-aminopyridine (219 mg, 1.26 mmol) in dichloromethane (5 mL) was stirred at ambient temperature for 40 min. To the mixture was added sodium triacetoxy borohydride (268 mg, 1.26 mmol) and acetic acid (0.01 mL) and stirring was for 40 hrs. The mixture was concentrated under reduced pressure and the residue was diluted with EtOAc. The mixture was washed with saturated aqueous sodium bicarbonate solution, brine, dried over sodium sulfate, filtered off and concentrated under reduced pressure. The residue was purified by column chromatography [silica gel, EtOAc/heptane=10/90 to 20/80] providing 6-bromo-N-(((2R,6S)-2,6-dimethyltetrahydro-2H-pyran-4-yl)methyl)pyridin-2-amine (110 mg) as colorless oil. LCMS (m/z): 299.0/301.0 [M+H]+; Rt=1.01 min.

### Synthesis of 5′-chloro-N6-(((2R,6S)-2,6-dimethyltetrahydro-2H-pyran-4-yl)methyl)-2,4′-bipyridine-2′,6-diamine

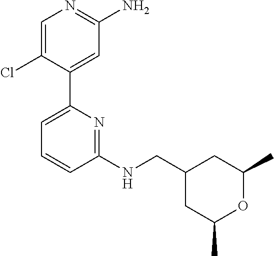

### Step 1: Preparation of 5′-chloro-N-(((2R,6S)-2,6-dimethyltetrahydro-2H-pyran-4-yl)methyl)-2′-fluoro-2,4′-bipyridin-6-amine

A mixture of 6-bromo-N-(((2R,6S)-2,6-dimethyltetrahydro-2H-pyran-4-yl)methyl)pyridin-2-amine (110 mg, 0.36 mmol), 5-chloro-2-fluoro-pyridine-4-boronic acid (193 mg, 1.10 mmol) in DME (2 mL) and 2M aqueous sodium carbonate solution (0.55 mL, 1.1 mmol) was purged with argon for 3 min. PdCl₂(dppf) CH₂Cl₂ (30 mg, 0.037 mmol) was added and the resulting mixture was heated at 95° C. for 3.5 hrs. The mixture was allowed to cool to room temperature and was diluted with EtOAc. The organic layer was washed with water and brine, dried over sodium sulfate, filtered off and concentrated under reduced pressure. The residue was purified by column chromatography [silica gel, EtOAc/heptane=10/90] providing 5′-chloro-N-(((2R,6S)-2,6-dimethyltetrahydro-2H-pyran-4-yl)methyl)-2′-fluoro-2,4′-bipyridin-6-amine (90 mg) as a colorless oil. Fractions were combined and concentrated under reduced pressure. LCMS (m/z): 350 (MH+), Rt=0.70 min.

### Step 2: Preparation of 5′-chloro-N6-(((2R,6S)-2,6-dimethyltetrahydro-2H-pyran-4-yl)methyl)-2,4′-bipyridine-2′,6-diamine

A mixture of 5′-chloro-N-(((2R,6S)-2,6-dimethyltetrahydro-2H-pyran-4-yl)methyl)-2′-fluoro-2,4′-bipyridin-6-amine (60 mg, 0.17 mmol) and aqueous ammonium hydroxide solution (28 wt. %, 3 mL) in DMSO (3 mL) was heated in a steel bomb at 130° C. for 17 hrs. The mixture was cooled to room temperature and was diluted with EtOAc. The mixture was washed with water, saturated aqueous sodium bicarbonate solution, and brine. The organic layer was dried over sodium sulfate, filtered off and concentrated under reduced pressure providing crude 5′-chloro-N6-(((2R,6S)-2,6-dimethyltetrahydro-2H-pyran-4-yl)methyl)-2,4′-bipyridine-2′,6-diamine (50 mg), which was directly used in the next reaction without further purification. LCMS (m/z): 347.1 [M+H]+; Rt=0.53 min.

### Synthesis of 6-bromo-N-((4-methyltetrahydro-2H-pyran-4-yl)methyl)pyridin-2-amine

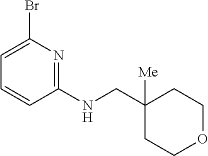

### Step 1: Preparation of 4-methyltetrahydro-2H-pyran-4-carbonitrile

To a solution of tetrahydro-2H-pyran-4-carbonitrile (2 g, 18.00 mmol) in tetrahydrofuran (10 mL) at 0-5° C. was added slowly LHMDS (21.59 mL, 21.59 mmol). The mixture was stirred for 1.5 hrs at 0° C. Iodomethane (3.37 mL, 54.0 mmol) was added slowly and stirring was continued for 30 min at ˜0° C. and then for ˜2 hrs at room temperature. The mixture was cooled to 0° C. and carefully diluted with 1N aqueous hydrochloride solution (30 mL) and EtOAc (5 mL) and concentrated under reduced pressure. The residue was taken up in diethylether and the separated organic layer was washed with brine, dried over sodium sulfate, filtered off and concentrated under reduced pressure providing crude 4-methyltetrahydro-2H-pyran-4-carbonitrile (1.8 g) as an orange oil, which was directly used in the next reaction without further purification. LCMS (m/z): 126.1 [M+H]+; Rt=0.44 min.

### Step 2: Preparation of (4-methyltetrahydro-2H-pyran-4-yl)methanamine

To a solution of 4-methyltetrahydro-2H-pyran-4-carbonitrile (1.8 g, 14.38 mmol) in tetrahydrofuran (30 mL) was carefully added lithium aluminum hydride (1M solution in tetrahydrofuran, 21.57 mL, 21.57 mmol) at 0° C. The reaction mixture was stirred for 15 min at 0° C., allowed to warm to room temperature and stirred for additional 3 hrs at room temperature. To the reaction mixture was carefully added water (0.9 mL) [Caution: gas development!], 1N aqueous sodium hydroxide solution (2.7 mL) and water (0.9 mL). The mixture was vigorously stirred for 30 min. The precipitate was filtered off and rinsed with tetrahydrofuran. The solution was concentrated under reduced pressure providing crude (4-methyltetrahydro-2H-pyran-4-yl)methanamine (1.54 g) as a yellowish solid, which was directly used in the next step without further purification. LCMS (m/z): 130.1 [M+H]+; Rt=0.21 min.

### Step 3: Preparation of 6-bromo-N-((4-methyltetrahydro-2H-pyran-4-yl)methyl)pyridin-2-amine

To a solution of 2-bromo-6-fluoropyridine (619 mg, 3.52 mmol) in DMSO (3 mL) was added (4-methyltetrahydro-2H-pyran-4-yl)methanamine (500 mg, 3.87 mmol) and triethylamine (498 mg, 4.93 mmol). The mixture was heated at 110° C. for 18 hrs. The mixture was allowed to cool to room temperature and diluted with EtOAc. The organic layer was washed with saturated aqueous sodium bicarbonate solution (1×), water (1×), brine (1×), dried over sodium sulfate, filtered off and concentrated under reduced pressure. The residue was purified by column chromatography [silica gel, 24 g, EtOAc/heptane=0/100 to 40/60] providing 6-bromo-N-((4-methyltetrahydro-2H-pyran-4-yl)methyl)pyridin-2-amine (750 mg) as a white solid. LCMS (m/z): 285.0/287.0 [M+H]+; Rt=0.88 min.

### Synthesis of 5′-chloro-N6-((4-methyltetrahydro-2H-pyran-4-yl)methyl)-2,4′-bipyridine-2′,6-diamine

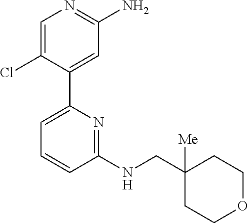

### Step 1: Preparation of 5′-chloro-2′-fluoro-N-((4-methyltetrahydro-2H-pyran-4-yl)methyl)-2,4′-bipyridin-6-amine

A mixture of 6-bromo-N-((4-methyltetrahydro-2H-pyran-4-yl)methyl)pyridin-2-amine (750 mg, 2.63 mmol), 5-chloro-2-fluoropyridin-4-ylboronic acid (830 mg, 4.73 mmol), PdCl₂(dppf) CH₂Cl₂ adduct (215 mg, 0.263 mmol) in DME (12 mL) and 2M aqueous sodium carbonate solution (4 mL, 8.00 mmol) was heated in a sealed tube at 103° C. for 4 hrs. The mixture was allowed to cool to room temperature and was diluted with EtOAc (˜50 mL) and saturated aqueous sodium bicarbonate solution. The separated organic layer was washed with saturated aqueous sodium bicarbonate solution (2×), dried over sodium sulfate, filtered off and concentrated under reduced pressure. The residue was purified by column chromatography [silica gel, 40 g, EtOAc/heptane=0/100 to 50/50] providing 5′-chloro-2′-fluoro-N-((4-methyltetrahydro-2H-pyran-4-yl)methyl)-2,4′-bipyridin-6-amine (691 mg) as a colorless oil. LCMS (m/z): 336.2 [M+H]+; Rt=0.66 min.

### Step 2: Preparation of 5′-chloro-N6-((4-methyltetrahydro-2H-pyran-4-yl)methyl)-2,4′-bipyridine-2′,6-diamine

A mixture of 5′-chloro-2′-fluoro-N-((4-methyltetrahydro-2H-pyran-4-yl)methyl)-2,4′-bipyridin-6-amine (350 mg, 1.042 mmol) and ammonium hydroxide (aqueous solution 30-35 wt. %, 16 mL) in DMSO (8 mL) was heated in a steel bomb at 140° C. for ˜24 hrs. The mixture was allowed to cool to room temperature and the mixture was diluted with water (˜75 mL) and EtOAc (˜75 mL). The separated organic layer was washed with saturated aqueous sodium bicarbonate solution (2×), dried over sodium sulfate, filtered off and concentrated under reduced pressure providing crude 5′-chloro-N6-((4-methyltetrahydro-2H-pyran-4-yl)methyl)-2,4′-bipyridine-2′,6-diamine (344 mg), which was directly used in the next reaction without further purification. LCMS (m/z): 333.1 [M+H]+; Rt=0.46 min.

### Synthesis of 6-bromo-N-((4-fluorotetrahydro-2H-pyran-4-yl)methyl)pyridin-2-amine

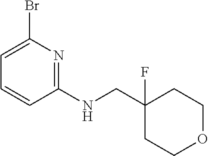

### Step 1: Preparation of 4-fluorotetrahydro-2H-pyran-4-carbaldehyde

Step 1a: To a solution of DIPEA (6.12 mL, 35.0 mmol) in dichloromethane (80 mL) was added trimethylsilyl trifluoromethanesulfonate (7.79 g, 35.0 mmol) and slowly a solution of tetrahydro-2H-pyran-4-carbaldehyde (2 g, 17.52 mmol) in dichloromethane (80 mL) at 0° C. Upon completion of the addition, the reaction mixture was stirred at room temperature for 2 hrs. The mixture was concentrated under reduced pressure and the residue was treated with hexane (200 mL). The precipitate was filtered off and the solution was concentrated under reduced pressure providing crude trimethylsilyl ether, which was directly used in the next step without further purification.

Step 1b: To a solution of crude trimethylsilyl ether in dichloromethane (100 mL) was added dropwise a solution of N-fluorobenzenesulfonimide (5.53 g, 17.52 mmol), dissolved in dichloromethane (50 mL), at 0° C. The mixture was stirred for 3 hrs at room temperature and the crude solution of 4-fluorotetrahydro-2H-pyran-4-carbaldehyde was directly used in the next reaction.

### Step 2: Preparation of 6-bromo-N-((4-fluorotetrahydro-2H-pyran-4-yl)methyl)pyridin-2-amine

To 6-bromopyridin-2-amine (3.03 g, 17.50 mmol) was added the crude solution of 4-fluorotetrahydro-2H-pyran-4-carbaldehyde in dichloromethane. To the resulting mixture was added acetic acid (1.002 mL, 17.50 mmol) and sodium triacetoxyborohydride (5.56 g, 26.3 mmol) in portions. The mixture was stirred for 2 hrs at room temperature. The mixture was diluted carefully with saturated aqueous sodium bicarbonate solution. The separated aqueous layer was extracted with dichloromethane (1×). The combined organic layers were washed with water (1×), saturated aqueous sodium bicarbonate solution (1×) and concentrated under reduced pressure. The solid residue was dissolved in dichloromethane (100 mL) and 3M aqueous hydrochloride solution (60 mL). The separated organic layer was extracted with 3M aqueous hydrochloride solution (3×20 mL). The combined acidic layers were washed with dichloromethane (1×). Solid sodium bicarbonate was added carefully to the acidic solution [Caution: gas development!] until pH>˜8. The aqueous mixture was extraction with dichloromethane (2×) and EtOAc (2×). The combined organic layers were concentrated under reduced pressure. The residue was dissolved in EtOAc. The solution was washed with 0.3M aqueous hydrochloride solution and brine, dried over sodium sulfate, filtered off and concentrated under reduced pressure. The residue was purified by column chromatography [silica gel, 40 g, EtOAc/heptane=5/95 to 30/70] providing 6-bromo-N-((4-fluorotetrahydro-2H-pyran-4-yl)methyl)pyridin-2-amine (1.82 g) as a white solid. LCMS (m/z): 288.9/291.0 [M+H]+; Rt=0.84 min.

### Synthesis of 5′-chloro-N6-((4-fluorotetrahydro-2H-pyran-4-yl)methyl)-2,4′-bipyridine-2′,6-diamine

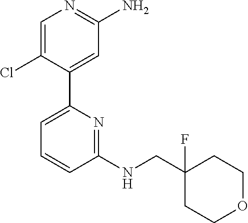

### Step 1: Preparation of 5′-chloro-2′-fluoro-N-((4-fluorotetrahydro-2H-pyran-4-yl)methyl)-2,4′-bipyridin-6-amine

A mixture of 6-bromo-N-((4-fluorotetrahydro-2H-pyran-4-yl)methyl)pyridin-2-amine (1 g, 3.46 mmol), 5-chloro-2-fluoropyridin-4-ylboronic acid (1.092 g, 6.23 mmol), PdCl₂(dppf) CH₂Cl₂ adduct (0.282 g, 0.346 mmol) in DME (13 mL) and 2M aqueous sodium carbonate solution (5.19 mL, 10.38 mmol) was heated in a sealed tube at 100° C. for 2 hrs. The mixture was allowed to cool to room temperature and was diluted with EtOAc (˜50 mL) and saturated aqueous sodium bicarbonate carbonate solution. The separated organic layer was washed with saturated aqueous sodium bicarbonate carbonate solution (2×), dried over sodium sulfate, filtered off and concentrated under reduced pressure. The residue was purified by column chromatography [silica gel, 80 g, EtOAc/heptane=5/95 to 50/50] providing 5′-chloro-2′-fluoro-N-((4-fluorotetrahydro-2H-pyran-4-yl)methyl)-2,4′-bipyridin-6-amine (1.00 g) as a colorless oil. LCMS (m/z): 340.1 [M+H]+; Rt=0.67 min.

### Step 2: Preparation of 5′-chloro-N6-((4-fluorotetrahydro-2H-pyran-4-yl)methyl)-2,4′-bipyridine-2′,6-diamine

A mixture of 5′-chloro-2′-fluoro-N-((4-fluorotetrahydro-2H-pyran-4-yl)methyl)-2,4′-bipyridin-6-amine (475 mg, 1.398 mmol) and ammonium hydroxide (aqueous solution 30-35 wt. %, 18 mL) in DMSO (12 mL) was heated in a steel bomb at 120° C. for 24 hrs. The mixture was allowed to cool to room temperature and the mixture was diluted with water and EtOAc. The separated organic layer was washed with saturated aqueous sodium bicarbonate solution (2×), dried over sodium sulfate, filtered off and concentrated under reduced pressure providing crude 5′-chloro-N6-((4-fluorotetrahydro-2H-pyran-4-yl)methyl)-2,4′-bipyridine-2′,6-diamine (450 mg), which was directly used in the next reaction without further purification. LCMS (m/z): 337.0 [M+H]+; Rt=0.49 min.

### Synthesis of 4-((6-bromopyridin-2-ylamino)methyl)tetrahydro-2H-pyran-4-carbonitrile

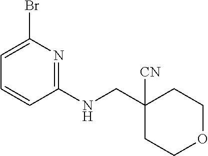

### Step 1: Preparation of dihydro-2H-pyran-4,4(3H)-dicarbonitrile

A mixture of malononitrile (0.991 g, 15 mmol), 1-bromo-2-(2-bromoethoxy)ethane (3.83 g, 16.50 mmol) and DBU (4.97 mL, 33.0 mmol) in DMF (6 mL) was heated at 85° C. for 3 hrs. The reaction mixture was cooled to room temperature and concentrated under reduced pressure. The residue was diluted with EtOAc (25 mL), washed with water (2×10 mL), dried over sodium sulfat, filtered off and concentrated under reduced pressure and further dried in high vacuo providing crude dihydro-2H-pyran-4,4(3H)-dicarbonitrile (1.65 g) as a light brown solid, which was directly used in the next step without further purification. GCMS: 136 [M]; Rt=5.76 min. ¹H NMR (300 MHz, chloroform-d) δ [ppm]: 2.14-2.32 (m, 4H) 3.77-3.96 (m, 4H).

### Step 2: Preparation of 4-(aminomethyl)tetrahydro-2H-pyran-4-carbonitrile

To a solution of dihydro-2H-pyran-4,4(3H)-dicarbonitrile (450 mg, 3.31 mmol in EtOH (15 mL) was added sodium borohydride (375 mg, 9.92 mmol) in portions and the mixture was stirred at room temperature for 4 hrs. The mixture was concentrated under reduced pressure and the residue was diluted with EtOAc (30 mL), washed with water (10 mL), dried over sodium sulfate, filtered off and concentrated under reduced pressure providing crude 4-(aminomethyl)tetrahydro-2H-pyran-4-carbonitrile (388 mg), which was directly used in the next step without further purification. LCMS (m/z): 141.0 [M+H]+; Rt=0.18 min.

### Step 3: Preparation of 4-((6-bromopyridin-2-ylamino)methyl)tetrahydro-2H-pyran-4-carbonitrile

To a solution of 2-bromo-6-fluoropyridine (400 mg, 2.273 mmol) in DMSO (4 mL) was added 4-(aminomethyl)tetrahydro-2H-pyran-4-carbonitrile (382 mg, 2.73 mmol) and triethylamine (0.792 mL, 5.68 mmol) sequentially at room temperature. The mixture was heated in a sealed glass bomb at 110° C. for 18 hrs. After being cooled to room temperature the reaction mixture was diluted with ethyl acetate (30 mL), washed with saturated sodium bicarbonate solution (10 mL) and brine (10 mL), dried over sodium sulfate and concentrated under reduced pressure. The residue was purified by column chromatography [silica gel, 12 g, EtOAc/heptane=5/95 to 20/80] providing 4-((6-bromopyridin-2-ylamino)methyl)tetrahydro-2H-pyran-4-carbonitrile (410 mg). LCMS (m/z): 297.9 [M+H]+; Rt=0.82 min. ¹H NMR (400 MHz, chloroform-d) 6 [ppm]: 1.67-1.96 (m, 4H) 3.59-3.78 (m, 4H) 3.98 (m, 2H) 4.82 (t, J=6.65 Hz, 1H), 6.39 (d, J=8.22 Hz, 1H) 6.72-6.84 (m, 1H) 7.16-7.33 (m, 1H).

### Synthesis of 4-((2′-amino-5′-chloro-2,4′-bipyridinyl-6-ylamino)methyl)tetrahydro-2H-pyran-4-carbonitrile

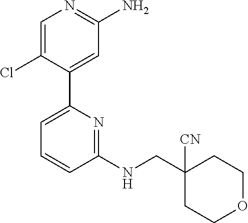

### Step 1: Preparation of 4-((5′-chloro-2′-fluoro-2,4′-bipyridinyl-6-ylamino)methyl)tetrahydro-2H-pyran-4-carbonitrile

A mixture of 4-((6-bromopyridin-2-ylamino)methyl)tetrahydro-2H-pyran-4-carbonitrile (410 mg, 1.38 mmol), 5-chloro-2-fluoropyridin-4-ylboronic acid (362.2 mg, 2.07 mmol), PdCl₂(dppf) CH₂Cl₂ adduct (113 mg, 0.14 mmol) in DME (5 mL) and 2M aqueous sodium carbonate solution (1.75 mL, 3.5 mmol) in a sealed tube was heated at 110° C. for 20 min using a microwave reactor. The mixture was allowed to cool to room temperature and was diluted with EtOAc (35 mL), filtered through celites and concentrated under reduced pressure. The residue was purified by column chromatography [silica gel, 24 g, EtOAc/heptane=5/95 to 50/50] providing 4-((5′-chloro-2′-fluoro-2,4′-bipyridinyl-6-ylamino)methyl)tetrahydro-2H-pyran-4-carbonitrile (360 mg). LCMS (m/z): 347.0 [M+H]+; Rt=0.81 min.

### Step 2: Preparation of 4-((2′-amino-5′-chloro-2,4′-bipyridinyl-6-ylamino)methyl)tetrahydro-2H-pyran-4-carbonitrile

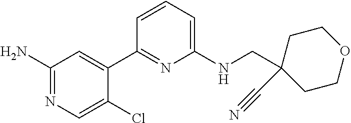

A mixture of 4-((5′-chloro-2′-fluoro-2,4′-bipyridinyl-6-ylamino)methyl)tetrahydro-2H-pyran-4-carbonitrile (180 mg, 0.519 mmol) and ammonium hydroxide (aqueous solution 30-35 wt. %, 2.5 mL) in DMSO (2.5 mL) was heated in a steel tube at 130° C. for ˜16 hrs. The mixture was cooled to room temperature and the mixture was diluted with EtOAc (25 mL). The mixture was washed with water (3×10 mL), dried over sodium sulfate, filtered off and concentrated under reduced pressure providing crude 4-((2′-amino-5′-chloro-2,4′-bipyridinyl-6-ylamino)methyl)tetrahydro-2H-pyran-4-carbonitrile (171 mg), which was directly used in the next reaction without further purification. LCMS (m/z): 344.0 [M+H]+; Rt=0.51 min.

### Synthesis of (6-bromo-5-chloro-pyridin-2-yl)-(4-methoxy-tetrahydro-pyran-4-ylmethyl)-carbamic acid tert-butyl ester

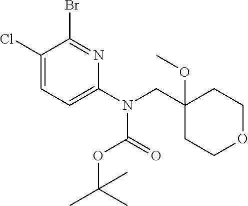

### Step 1: Preparation of 1,6-dioxaspiro[2.5]octane

To a solution of trimethylsulfonium iodide (3.27 g, 16 mmol) in DMSO (20 mL) under nitrogen atmosphere was added dihydro-2H-pyran-4(3H)-one (1.0 g, 10 mmol). To the mixture was added slowly a solution of tert-butoxide (1.68 g, 15 mmol) in DMSO (15 mL) and the solution was stirred at room temperature overnight. The reaction mixture was diluted slowly with water (50 mL) and extracted with diethylether (3×20 mL). The combined organic layers were dried over sodium sulfate, filtered off and concentrated under reduced pressure providing crude 1,6-dioxaspiro[2.5]octane (650 mg), which was directly used without further purification. ¹H NMR (300 MHz, chloroform-d) δ [ppm]: 1.44-1.62 (m, 2H) 1.76-1.98 (m, 2H) 2.70 (s, 2H) 3.70-3.98 (m, 4H).

### Step 2: Preparation of (4-methoxytetrahydro-2H-pyran-4-yl) MeOH

To a solution of 1,6-dioxaspiro[2.5]octane (600 mg, 5.26 mmol) in MeOH (10 mL) under nitrogen was added camphorsulfonic acid (50 mg, 0.21 mmol) at 0° C. and the mixture was stirred at 0° C. for 2 hrs. The mixture was concentrated under reduced pressure providing crude (4-methoxytetrahydro-2H-pyran-4-yl)methanol (707 mg) as a light yellow oil, which was directly used in the next step without further purification. ¹H NMR (300 MHz, chloroform-d) δ [ppm]: 1.89-2.08 (m, 4H), 3.18-3.30 (m, 3H), 3.47-3.59 (m, 2H), 3.64-3.78 (m, 4H).

### Step 3: Preparation of toluene-4-sulfonic acid 4-methoxy-tetrahydro-pyran-4-ylmethyl ester

To a solution of (4-methoxytetrahydro-2H-pyran-4-yl) MeOH (300 mg, 2.05 mmol) in pyridine (4 mL) was added toluenesulfonic chloride (430 mg, 2.25 mmol) at room temperature and the mixture was stirred at 25° C. overnight. The mixture was concentrated under reduced pressure and the residue was dissolved in dichloromethane (2 mL). Purification by column chromatography [silica gel, 12 g, EtOAc/hexane=0/100 to 30/70] provided toluene-4-sulfonic acid 4-methoxy-tetrahydro-pyran-4-ylmethyl ester (360 mg) as a light yellow solid. ¹H NMR (300 MHz, chloroform-d) δ [ppm]: 1.45-1.63 (m, 2H) 1.61-1.79 (m, 2H) 2.46 (s, 3H), 3.16 (s, 3H) 3.53-3.75 (m, 4H) 3.93 (s, 2H), 7.36 (d, J=8.20 Hz, 2H) 7.81 (d, J=8.20 Hz, 2H).

### Step 4: Preparation of (6-bromo-5-chloro-pyridin-2-yl)-(4-methoxy-tetrahydro-pyran-4-ylmethyl)-carbamic acid tert-butyl ester

To a solution of tert-butyl 6-bromo-5-chloropyridin-2-ylcarbamate (140 mg, 0.455 mmol) in DMF (2 mL) under nitrogen was added sodium hydride (60 wt. %, 30 mg, 0.774 mmol) and the mixture was stirred at room temperature for 1 hr. To the mixture was added a solution of toluene-4-sulfonic acid 4-methoxy-tetrahydro-pyran-4-ylmethyl ester (164 mg, 0.546 mmol) in DMF (1.5 mL) stirring was continued at 85° C. overnight. The reaction mixture was diluted with EtOAc (30 mL), washed with water (3×20 mL), dried over sodium sulfate, filtered off and concentrated under reduced pressure. The residue was purified by column chromatography [silica gel, 12 g, EtOAc/hexane=5/95 to 20/80] providing (6-bromo-5-chloro-pyridin-2-yl)-(4-methoxy-tetrahydro-pyran-4-ylmethyl)-carbamic acid tert-butyl ester (92 mg) as a viscous oil, which solidified overnight. LCMS (m/z): 437.0 [M+H]+; Rt=1.16 min.

### Synthesis of 3,5′-dichloro-N6-((4-methoxytetrahydro-2H-pyran-4-yl)methyl)-2,4′-bipyridine-2′,6-diamine

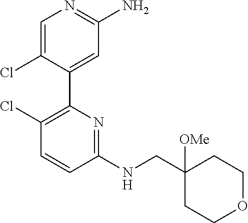

### Step 1: Preparation of (3,5′-dichloro-2′-fluoro-[2,4′]bipyridinyl-6-yl)-(4-methoxy-tetrahydro-pyran-4-ylmethyl)-carbamic acid tert-butyl ester

A mixture of tert-butyl 6-bromo-5-chloropyridin-2-yl((4-methoxytetrahydro-2H-pyran-4-yl)methyl)carbamate (40 mg, 0.092 mmol), 5-chloro-2-fluoropyridin-4-ylboronic acid (32.2 mg, 0.184 mmol), PdCl₂(dppf) CH₂Cl₂ adduct (11.3 mg, 0.014 mmol) in DME (1 mL) and 2M aqueous sodium carbonate solution (0.2 mL, 0.4 mmol) in a sealed tube was heated at 100° C. for 3 hrs. The mixture was allowed to cool to room temperature and was diluted with EtOAc (15 mL), filtered trough celites and concentrated under reduced pressure. The residue was purified by column chromatography [silica gel, 12 g, EtOAc/hexane=5/95 to 50/50] providing (3,5′-dichloro-2′-fluoro-[2,4′]bipyridinyl-6-yl)-(4-methoxy-tetrahydro-pyran-4-ylmethyl)-carbamic acid tert-butyl ester (30 mg). LCMS (m/z): 486.2 [M+H]+; Rt=1.16 min.

### Step 2: Preparation of 3,5′-dichloro-N6-((4-methoxytetrahydro-2H-pyran-4-yl)methyl)-2,4′-bipyridine-2′,6-diamine

A mixture of (3,5′-dichloro-2′-fluoro-[2,4′]bipyridinyl-6-yl)-(4-methoxy-tetrahydro-pyran-4-ylmethyl)-carbamic acid tert-butyl ester (90 mg, 0.185 mmol) and aqueous ammonium hydroxide solution (30 wt. %, 1.5 mL) in DMSO (1.5 mL) was heated in a steel tube at 140° C. for ˜16 hrs. The mixture was allowed to cool to room temperature and was diluted with EtOAc (25 mL). The mixture was washed with water (3×10 mL), dried over sodium sulfate, filtered off and concentrated under reduced pressure providing crude 3,5′-dichloro-N6-((4-methoxytetrahydro-2H-pyran-4-yl)methyl)-2,4′-bipyridine-2′,6-diamine (50 mg), which was directly used in the next reaction without further purification. LCMS (m/z): 383.1 [M+H]+; Rt=0.60 min.

### Synthesis of 5-fluoro-6-(((4-methyltetrahydro-2H-pyran-4-yl)methyl)amino)pyridin-2-yl trifluoromethanesulfonate

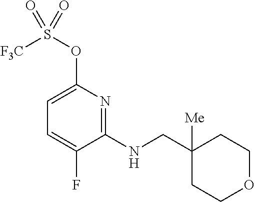

### Step 1: Preparation of 3,6-difluoro-N-((4-methyltetrahydro-2H-pyran-4-yl)methyl)pyridin-2-amine

A mixture of 2,3,6-trifluoropyridine (858 mg, 6.45 mmol), (4-methyltetrahydro-2H-pyran-4-yl)methanamine (1.0 g, 7.74 mmol) and triethylamine (2.16 mL, 15.5 mmol) in NMP (16 mL) was heated at 70° C. for 1 hr. The reaction mixture was cooled to room temperature and was diluted with EtOAc (˜100 mL), brine (˜50 mL) and water (˜50 mL). The separated organic layer was washed with brine (1×), 0.3N aqueous hydrochloride solution (2×), saturated aqueous sodium bicarbonate solution (1×), brine (1×), dried over sodium sulfate, filtered off and concentrated under reduced pressure providing crude 3,6-difluoro-N-((4-methyltetrahydro-2H-pyran-4-yl)methyl)pyridin-2-amine (1.4 g) as a colorless oil, which was directly used in the next reaction without further purification. LCMS (m/z): 243.1 [M+H]+; Rt=0.86 min.

### Step 2: Preparation of 3-fluoro-6-methoxy-N-((4-methyltetrahydro-2H-pyran-4-yl)methyl)pyridin-2-amine

To a solution of 3,6-difluoro-N-((4-methyltetrahydro-2H-pyran-4-yl)methyl)pyridin-2-amine (1.4 g, 5.78 mmol) in MeOH (14 mL) was added sodium methoxide (25 wt. % in MeOH, 7 mL, 30.8 mmol). The mixture was heated in a steel bomb at 135° C. for 3 d. The mixture was allowed to cool to room temperature and concentrated under reduced pressure. The residue was taken up in water (200 mL). The formed precipitate was filtered off and rinsed with water. The solid was dissolved in dichloromethane. The organic solution was washed with brine, dried over sodium sulfate, filtered off and concentrated under reduced pressure. The residue was purified by column chromatography [silica gel, 80 g, 20 min, EtOAc/heptane=0/100 to 25/75] providing 3-fluoro-6-methoxy-N-((4-methyltetrahydro-2H-pyran-4-yl)methyl)pyridin-2-amine (1.22 g) as an off-white solid. LCMS (m/z): 255.1 [M+H]+; Rt=0.89 min.

### Step 3: Preparation of 5-fluoro-6-(((4-methyltetrahydro-2H-pyran-4-yl)methyl)amino)pyridin-2-ol

To a solution of 3-fluoro-6-methoxy-N-((4-methyltetrahydro-2H-pyran-4-yl)methyl)pyridin-2-amine in acetonitrile (12 mL) was added sodium iodide (4.24 g, 28.3 mmol) and slowly chlorotrimethylsilane (3.62 mL, 28.3 mmol). The mixture was heated to reflux (oil bath: 83° C.) for 4 hrs. The mixture was allowed to cool to room temperature and was diluted with EtOAc and saturated aqueous sodium bicarbonate solution. The mixture was vigorously stirred for 15 min and acidified with 0.5N aqueous NaHSO₄ solution and stirring was continued for 5 min. The mixture was neutralized with saturated aqueous sodium bicarbonate solution. The separated aqueous phase was extracted with EtOAc (3×). The combined organic layers were dried over sodium sulfate, filtered off and concentrated under reduced pressure. The residue was purified by column chromatography [silica gel, 40 g, 25 min, EtOAc/heptane=5/95 to 50/50] providing 5-fluoro-6-(((4-methyltetrahydro-2H-pyran-4-yl)methyl)amino)pyridin-2-ol (420 mg) as colorless highly viscous oil. LCMS (m/z): 241.1 [M+H]+; Rt=0.55 min.

### Alternative preparation of 5-fluoro-6-(((4-methyltetrahydro-2H-pyran-4-yl)methyl)amino)pyridin-2-ol

### Step 2-a: Preparation of 6-(benzyloxy)-3-fluoro-N-((4-methyltetrahydro-2H-pyran-4-yl)methyl)pyridin-2-amine

To a solution of benzyl alcohol (13.48 mL, 14.09 g, 130 mmol) in anhydrous

DMF (200 mL) under argon was carefully added sodium hydride (60 wt. % in mineral oil, 5.21 g, 130 mmol). The mixture was stirred at room temperature for 15 min, 3,6-difluoro-N-((4-methyltetrahydro-2H-pyran-4-yl)methyl)pyridin-2-amine (10.52 g, 43.4 mmol) was added and stirring was continued at 90° C. for 14 hrs. The reaction mixture was allowed to cool to room temperature, poured into brine (200 mL) and extracted with EtOAc (3×200 mL). The combined extracts were washed with water (3×200 mL), brine (1×200 mL), dried over sodium sulfate, filtered off and concentrated under reduced pressure. The residue was purified by column chromatography [silica gel, 240 g, 25 min, EtOAc/hexane=10/90 to 50/50] providing 6-(benzyloxy)-3-fluoro-N-((4-methyltetrahydro-2H-pyran-4-yl)methyl)pyridin-2-amine (12.15 g). LCMS (m/z): 331.1 [M+H]+; Rt=1.15 min.

### Step 3-a: Preparation of 5-fluoro-6-(((4-methyltetrahydro-2H-pyran-4-yl)methyl)amino)pyridin-2-ol

A solution of 6-(benzyloxy)-3-fluoro-N-((4-methyltetrahydro-2H-pyran-4-yl)methyl)pyridin-2-amine (12.15 g, 36.8 mmol) in EtOH (450 mL) was placed under argon and Pd/C (10 wt. %, 1.96 g) was added. The mixture was stirred under hydrogen atmosphere (˜1 atm, balloon) for 15 hrs. The reaction mixture was filtered through a pad of celites and the filtrate was concentrated under reduced pressure providing crude 5-fluoro-6-(((4-methyltetrahydro-2H-pyran-4-yl)methyl)amino)pyridin-2-ol (8.30 g), which was directly used in the next step without further purification. LCMS (m/z): 241.0 [M+H]+; Rt=0.51 min.

### Step 4: Preparation of 5-fluoro-6-(((4-methyltetrahydro-2H-pyran-4-yl)methyl)amino)pyridin-2-yl trifluoromethanesulfonate

To a solution of 5-fluoro-6-(((4-methyltetrahydro-2H-pyran-4-yl)methyl)amino)pyridin-2-ol (420 mg, 1.748 mmol) and triethylamine (0.731 mL, 5.24 mmol) in dichloromethane (16 mL) was added slowly trifluoromethanesulfonic anhydride (0.443 mL, 2.62 mmol) at 0° C. The mixture was stirred for 2 hrs at 0° C. and poured carefully into ice-cooled saturated aqueous sodium bicarbonate solution. The separated aqueous layer was extracted with dichloromethane (2×). The combined organic layers were dried over sodium sulfate, filtered off and concentrated under reduced pressure. The residue was purified by column chromatography [silica gel, 24 g, EtOAc/heptane=5/95 to 40/60] providing 5-fluoro-6-(((4-methyltetrahydro-2H-pyran-4-yl)methyl)amino)pyridin-2-yl trifluoromethanesulfonate (600 mg) as colorless oil.

### Synthesis of 5′-chloro-5-fluoro-N6-((4-methyltetrahydro-2H-pyran-4-yl)methyl)-2,4′-bipyridine-2′,6-diamine

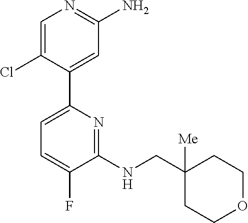

### Step 1: Preparation of 5′-chloro-2′,5-difluoro-N-((4-methyltetrahydro-2H-pyran-4-yl)methyl)-2,4′-bipyridin-6-amine

A mixture of 5-fluoro-6-(((4-methyltetrahydro-2H-pyran-4-yl)methyl)amino)pyridin-2-yl trifluoromethanesulfonate (600 mg, 1.611 mmol), 5-chloro-2-fluoropyridin-4-ylboronic acid (565 mg, 3.22 mmol), PdCl₂(dppf) CH₂Cl₂ adduct (132 mg, 0.161 mmol) in DME (8 mL) and 2M aqueous sodium carbonate solution (3 mL, 6.00 mmol) in a sealed tube was heated at 102° C. for 10 hrs. The mixture was allowed to cool to room temperature and was diluted with EtOAc (˜100 mL) and saturated aqueous sodium bicarbonate solution. The separated organic layer was washed with saturated aqueous sodium bicarbonate solution (2×), dried over sodium sulfate, filtered off and concentrated under reduced pressure. The residue was purified by column chromatography [silica gel, 40 g, EtOAc/heptane=0/100 to 30/70] providing 5′-chloro-2′,5-difluoro-N-((4-methyltetrahydro-2H-pyran-4-yl)methyl)-2,4′-bipyridin-6-amine (490 mg) as a colorless oil. LCMS (m/z): 354.2 [M+H]+; Rt=1.05 min.

### Step 2: Preparation of 5′-chloro-5-fluoro-N6-((4-methyltetrahydro-2H-pyran-4-yl)methyl)-2,4′-bipyridine-2′,6-diamine

A mixture of 5′-chloro-2′,5-difluoro-N-((4-methyltetrahydro-2H-pyran-4-yl)methyl)-2,4′-bipyridin-6-amine (250 mg, 0.707 mmol) and ammonium hydroxide (aqueous solution 30-35 wt. %, 16 mL) in DMSO (8 mL) was heated in a steel bomb at 140° C. for ˜18 hrs. The mixture was allowed to cool to room temperature and was diluted with water (˜75 mL) and EtOAc (˜75 mL). The separated organic layer was washed with saturated aqueous sodium bicarbonate solution (2×), dried over sodium sulfate, filtered off and concentrated under reduced pressure providing crude 5′-chloro-5-fluoro-N6-((4-methyltetrahydro-2H-pyran-4-yl)methyl)-2,4′-bipyridine-2′,6-diamine (246 mg), which was directly used in the next reaction without further purification. LCMS (m/z): 351.0 [M+H]+; Rt=0.65 min.

### Synthesis of 6-(((4-ethyltetrahydro-2H-pyran-4-yl)methyl)amino)-5-fluoropyridin-2-yl trifluoromethanesulfonate

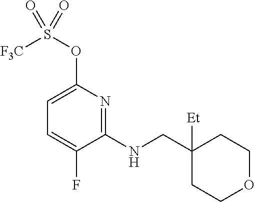

### Step 1: Preparation of N-((4-ethyltetrahydro-2H-pyran-4-yl)methyl)-3,6-difluoropyridin-2-amine

A mixture of 2,3,6-trifluoropyridine (774 mg, 5.82 mmol), (4-ethyltetrahydro-2H-pyran-4-yl)methanamine (1000 mg, 6.98 mmol) and triethylamine (1.946 mL, 13.96 mmol) in NMP (16 mL) was heated at 70° C. for 1 hr. The reaction mixture was cooled to room temperature and was diluted with EtOAc (˜100 mL), brine (˜50 mL) and water (˜50 mL). The separated organic layer was washed with brine (1×), 0.3N aqueous hydrochloride solution (2×), saturated aqueous sodium bicarbonate solution (1×), brine (1×), dried over sodium sulfate, filtered off and concentrated under reduced pressure providing crude N-((4-ethyltetrahydro-2H-pyran-4-yl)methyl)-3,6-difluoropyridin-2-amine (1.35 g) as colorless oil, which was directly used in the next reaction without further purification. LCMS (m/z): 257.2 [M+H]+; Rt=0.94 min.

### Step 2: Preparation of N-((4-ethyltetrahydro-2H-pyran-4-yl)methyl)-3-fluoro-6-methoxypyridin-2-amine

To a solution of N-((4-ethyltetrahydro-2H-pyran-4-yl)methyl)-3,6-difluoropyridin-2-amine (1.5 g, 5.85 mmol) in MeOH (15 mL) was added sodium methoxide (˜25 wt. % in MeOH, 7.09 mL, 31.2 mmol). The mixture was heated in a steel bomb at 135° C. for 3 days. The mixture was allowed to cool to room temperature and concentrated under reduced pressure. The residue was taken up in water (200 mL). The formed precipitate was filtered off and rinsed with water. The solid was dissolved in dichloromethane and the organic solution was washed with brine, dried over sodium sulfate, filtered off and concentrated under reduced pressure providing crude N-((4-ethyltetrahydro-2H-pyran-4-yl)methyl)-3-fluoro-6-methoxypyridin-2-amine (1.26 g) as an orange oil, which was directly used in the next reaction without further purification. LCMS (m/z): 269.2 [M+H]+; Rt=0.99 min.

### Step 3: Preparation of 6-(((4-ethyltetrahydro-2H-pyran-4-yl)methyl)amino)-5-fluoropyridin-2-ol

To a solution of N-((4-ethyltetrahydro-2H-pyran-4-yl)methyl)-3-fluoro-6-methoxypyridin-2-amine (1.26 g, 4.70 mmol) in acetonitrile (13 mL) was added sodium iodide (4.22 g, 28.2 mmol) and slowly chlorotrimethylsilane (3.60 mL, 28.2 mmol). The mixture was heated to reflux (oil bath: 83° C.) for 4 hrs. The mixture was allowed to cool to room temperature and was diluted with EtOAc and saturated aqueous sodium bicarbonate solution and was vigorously stirred for 15 min. The mixture was acidified with 0.5N aqueous NaHSO4 solution and stirring was continued for 5 min. The mixture was neutralized with saturated aqueous sodium bicarbonate solution. The separated aqueous phase was extracted with EtOAc (3×). The combined organic layers were dried over sodium sulfate, filtered off and concentrated under reduced pressure. The residue was purified by column chromatography [silica gel, 40 g, 25 min, EtOAc/heptane=5/95 to 50/50] providing 6-(((4-ethyltetrahydro-2H-pyran-4-yl)methyl)amino)-5-fluoropyridin-2-ol (480 mg) as a colorless highly viscous oil. LCMS (m/z): 255.1 [M+H]+; Rt=0.64 min.

### Step 4: Preparation of 6-(((4-ethyltetrahydro-2H-pyran-4-yl)methyl)amino)-5-fluoropyridin-2-yl trifluoromethanesulfonate

To a solution of 6-(((4-ethyltetrahydro-2H-pyran-4-yl)methyl)amino)-5-fluoropyridin-2-ol (480 mg, 1.888 mmol) and triethylamine (0.789 mL, 5.66 mmol) in dichloromethane (19 mL) was added slowly trifluoromethanesulfonic anhydride (0.478 mL, 2.83 mmol) at 0° C. The mixture was stirred for 2 hrs at 0° C. and poured carefully into ice-cooled saturated aqueous sodium bicarbonate solution. The separated aqueous layer was extracted with dichloromethane (2×). The combined organic layers were dried over sodium sulfate, filtered off and concentrated under reduced pressure. The residue was purified by column chromatography [silica gel, 24 g, 20 min, EtOAc/heptane=5/95 to 40/60] providing 6-(((4-ethyltetrahydro-2H-pyran-4-yl)methyl)amino)-5-fluoropyridin-2-yl trifluoromethanesulfonate (685 mg) as yellow oil.

### Synthesis of 5′-chloro-N6-((4-ethyltetrahydro-2H-pyran-4-yl)methyl)-5-fluoro-2,4′-bipyridine-2′,6-diamine

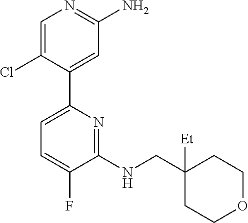

### Step 1: Preparation of 5′-chloro-N-((4-ethyltetrahydro-2H-pyran-4-yl)methyl)-2′,5-difluoro-2,4′-bipyridin-6-amine

A mixture of 6-(((4-ethyltetrahydro-2H-pyran-4-yl)methyl)amino)-5-fluoropyridin-2-yl trifluoromethanesulfonate (685 mg, 1.773 mmol), 5-chloro-2-fluoropyridin-4-ylboronic acid (622 mg, 3.55 mmol), PdCl₂(dppf).CH₂Cl₂ adduct (145 mg, 0.177 mmol) in DME (8 mL) and 2M sodium carbonate solution (3 mL, 6.0 mmol) in a sealed tube was heated at 95° C. for 3 hrs. The mixture was allowed to cool to room temperature and was diluted with EtOAc (˜100 mL) and saturated aqueous sodium bicarbonate carbonate solution. The separated organic layer was washed with saturated aqueous sodium bicarbonate carbonate solution (2×), dried over sodium sulfate, filtered off and concentrated under reduced pressure. The residue was purified by column chromatography [silica gel, 40 g, EtOAc/heptane=0/100 to 30/70] providing 5′-chloro-N-((4-ethyltetrahydro-2H-pyran-4-yl)methyl)-2′,5-difluoro-2,4′-bipyridin-6-amine (539 mg) as a white solid. Fractions were combined and concentrated under reduced pressure. LCMS (m/z): 368.2 [M+H]+; Rt=1.12 min.

### Step 2: Preparation of 5′-chloro-N6-((4-ethyltetrahydro-2H-pyran-4-yl)methyl)-5-fluoro-2,4′-bipyridine-2′,6-diamine

A mixture of 5′-chloro-N-((4-ethyltetrahydro-2H-pyran-4-yl)methyl)-2′,5-difluoro-2,4′-bipyridin-6-amine (255 mg, 0.693 mmol) and ammonium hydroxide (aqueous solution 30-35 wt. %, 16 mL) in DMSO (8 mL) was heated in a steel bomb at 140° C. for ˜18 hrs. The mixture was allowed to cool to room temperature and was diluted with water (˜75 mL) and EtOAc (˜75 mL). The separated organic layer was washed with saturated aqueous sodium bicarbonate solution (2×), dried over sodium sulfate, filtered off and concentrated under reduced pressure providing crude 5′-chloro-N6-((4-ethyltetrahydro-2H-pyran-4-yl)methyl)-5-fluoro-2,4′-bipyridine-2′,6-diamine (256 mg), which was directly used in the next reaction without further purification. LCMS (m/z): 365.0 [M+H]+; Rt=0.71 min.

### Synthesis of 5-fluoro-6-(((4-methoxytetrahydro-2H-pyran-4-yl)methyl)amino)pyridin-2-yl trifluoromethanesulfonate

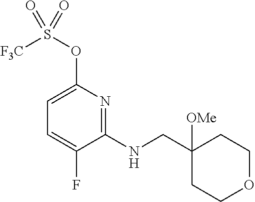

### Step 1: Preparation of 4,4-dimethoxytetrahydro-2H-pyran

A mixture of dihydro-2H-pyran-4(3H)-one (501 mg, 5 mmol), trimethyl orthoformate (0.608 mL, 5.50 mmol) and toluenesulfonic acid monohydrate (2.85 mg, 0.015 mmol) in MeOH (1 mL) was stirred in a sealed tube at 80° C. for 30 min. The reaction mixture was allowed to cool to room temperature and was concentrated under reduced pressure providing crude 4,4-dimethoxytetrahydro-2H-pyran (703 mg), which was used in the next step without further purification. ¹H NMR (400 MHz, chloroform-d) δ [ppm]: 1.61-1.90 (m, 4H) 3.20 (s, 6H) 3.60-3.78 (m, 4H).

### Step 2: Preparation of 4-methoxytetrahydro-2H-pyran-4-carbonitrile

To a solution of 4,4-dimethoxytetrahydro-2H-pyran (0.703 g, 4.81 mmol) and tin(IV)chloride (0.564 mL, 4.81 mmol) in dichloromethane (15 mL) was added slowly 2-isocyano-2-methylpropane (0.400 g, 4.81 mmol) at −70° C. and the mixture was allowed to warm to room temperature over 2-3 hrs. The mixture was diluted with aqueous sodium bicarbonate solution (10 mL) and dichloromethane (20 mL). The separated organic layer was washed with water (3×10 mL) and dried over sodium sulfate, filtered off and concentrated under reduced pressure providing crude 4-methoxytetrahydro-2H-pyran-4-carbonitrile (511 mg), which was used in the next step without further purification. GCMS: 109 [M-MeOH]; Rt=5.44 min.

### Step 3: Preparation of (4-methoxytetrahydro-2H-pyran-4-yl)methanamine

To a mixture of LiAlH₄ (275 mg, 7.24 mmol) in tetrahydrofuran (10 mL) at room temperature was slowly added a solution of 4-methoxytetrahydro-2H-pyran-4-carbonitrile (511 mg, 3.62 mmol) in tetrahydrofuran (10 mL). The mixture was stirred at room temperature for 1 hr and heated to reflux for 3 hrs. The reaction mixture was cooled to 0° C. and water (3 mL) was carefully added dropwise. The resulting mixture was stirred for additional 30 min and filtered to remove all solids. The filtrate was dried over sodium sulfate for 2 hrs, filtered off and concentrated under reduced pressure providing crude (4-methoxytetrahydro-2H-pyran-4-yl)methanamine (370 mg), which was used in the next step without further purification. LCMS (m/z): 146.1 [M+H]+, 114.0 [M-MeOH]; Rt=0.19 min.

### Step 4: Preparation of 3,6-difluoro-N-((4-methoxytetrahydro-2H-pyran-4-yl)methyl)pyridin-2-amine

A mixture of 2,3,6-trifluoropyridine (280 mg, 2.104 mmol), crude (4-methoxytetrahydro-2H-pyran-4-yl)methanamine (367 mg, 2.52 mmol) and triethylamine (0.704 mL, 5.05 mmol) in NMP (5 mL) was heated at 75° C. for 1 hr. The reaction mixture was cooled to room temperature and was diluted with EtOAc (˜30 mL), brine (˜20 mL) and water (˜10 mL). The separated organic layer was washed with brine (10 mL), water (10 mL), dried with sodium sulfate, filtered off and concentrated under reduced pressure. The residue was purified by column chromatography [silica gel, 12 g, 20 min, EtOAc/heptane=0/100 to 30/70]. Fractions were combined and concentrated under reduced pressure providing 3,6-difluoro-N-((4-methoxytetrahydro-2H-pyran-4-yl)methyl)pyridin-2-amine (470 mg). LCMS (m/z): 259.0 [M+H]+; Rt=0.78 min. 1H NMR (400 MHz, chloroform-d) δ [ppm]: 1.52-1.72 (m, 2H) 1.73-1.91 (m, 2H) 3.16-3.31 (m, 3H), 3.51 (d, J=5.09 Hz, 2H) 3.64-3.81 (m, 4H) 4.88 (br. s., 1H) 5.94-6.12 (m, 1H) 7.19 (ddd, J=9.78, 8.22, 6.26 Hz, 1H).

### Step 5: Preparation of 6-(benzyloxy)-3-fluoro-N-((4-methoxytetrahydro-2H-pyran-4-yl)methyl)pyridin-2-amine

To a solution of benzyl alcohol (314 mg, 2.90 mmol) in anhydrous DMF (2 mL) under argon was added carefully sodium hydride (60 wt. % in mineral oil, 69.7 mg). The mixture was stirred at room temperature for 15 min and a solution of 3,6-difluoro-N-((4-methoxytetrahydro-2H-pyran-4-yl)methyl)pyridin-2-amine (250 mg, 0.968 mmol) in anhydrous DMF (2 mL) was added and stirring was continued at 90° C. for 3 hrs. The reaction mixture was allowed to cool to room temperature and carefully poured into brine (20 mL). The mixture was extracted with EtOAc (3×10 mL) and the combined extracts were washed with water (3×10 mL) and brine (1×10 mL). The organic layer was dried over sodium sulfate, filtered off and concentrated under reduced pressure. The residue was purified by column chromatography [silica gel, 12 g, EtOAc/hexane=0/100 to 30/70] providing 6-(benzyloxy)-3-fluoro-N-((4-methoxytetrahydro-2H-pyran-4-yl)methyl)pyridin-2-amine (310 mg). LCMS (m/z): 347.3 [M+H]+; Rt=1.07 min.

### Step 6: Preparation of 5-fluoro-6-(((4-methoxytetrahydro-2H-pyran-4-yl)methyl)amino)pyridin-2-ol

A mixture of 6-(benzyloxy)-3-fluoro-N-((4-methoxytetrahydro-2H-pyran-4-yl)methyl)pyridin-2-amine (105 mg, 0.303 mmol), ammonium formate (57.3 mg, 0.909 mmol) and Pd/C (10 wt. %, water 50 wt. %, 15 mg) in MeOH (1 mL) was stirred at 70° C. for 30 min. The reaction mixture was allowed to cool to room temperature and additional Pd/C (10 wt. %, water 50 wt. %, 10 mg) and ammonium formate (50 mg) were added and the reaction mixture was stirred at 70° C. for an additional hour. The mixture was then filtered to remove solids and the filterate was concentrated under reduced pressure and dried further in high vacuo providing crude 5-fluoro-6-(((4-methoxytetrahydro-2H-pyran-4-yl)methyl)amino)pyridin-2-ol (79 mg). LCMS (m/z): 257.0 [M+H]+; Rt=0.51 min.

### Step 7: Preparation of 5-fluoro-6-(((4-methoxytetrahydro-2H-pyran-4-yl)methyl)amino)pyridin-2-yl trifluoromethanesulfonate

To a solution of 5-fluoro-6-(((4-methoxytetrahydro-2H-pyran-4-yl)methyl)amino)pyridin-2-ol (77 mg, 0.3 mmol) and triethylamine (0.418 mL, 3.00 mmol) in dichloromethane (4 mL) was slowly added trifluoromethanesulfonic anhydride (0.076 mL, 0.450 mmol) at 0° C. The reaction mixture was stirred for 2 hrs at 0° C. and 1 hr at room temperature. The mixture was poured carefully into ice-cooled saturated aqueous sodium bicarbonate solution. The separated aqueous layer was extracted with dichloromethane (2×15 mL). The combined organic layers were dried over sodium sulfate, filtered off and concentrated under reduced pressure. The residue was purified by column chromatography [silica gel, 12 g, EtOAc/heptane=5/95 to 40/60]. Pure fractions were combined and concentrated under reduced pressure providing 5-fluoro-6-(((4-methoxytetrahydro-2H-pyran-4-yl)methyl)amino)pyridin-2-yl trifluoromethanesulfonate (50 mg) as a colorless oil. LCMS (m/z): 389.0 [M+H]+; Rt=1.01 min.

### Synthesis of 5′-chloro-5-fluoro-N6-((4-methoxytetrahydro-2H-pyran-4-yl)methyl)-2,4′-bipyridine-2′,6-diamine

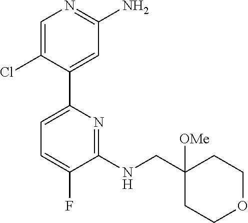

### Step 1: Preparation of tert-butyl 5′-chloro-5-fluoro-6-(((4-methoxytetrahydro-2H-pyran-4-yl)methyl)amino)-2,4′-bipyridinyl-2′-ylcarbamate

A mixture of 5-fluoro-6-((4-methoxytetrahydro-2H-pyran-4-yl)methylamino) pyridin-2-yl trifluoromethanesulfonate (50 mg, 0.129 mmol), 2-(tert-butoxycarbonylamino)-5-chloropyridin-4-ylboronic acid (70.2 mg, 0.258 mmol), PdCl₂(dppf) CH₂Cl₂ adduct (21.03 mg, 0.026 mmol) in DME (1.5 mL) and aqueous sodium carbonate solution (54.6 mg in 0.5 mL of water) was degassed with argon and heated in a sealed tube at 110° C. for 20 min in a microwave reactor. The mixture was allowed to cool to room temperature. The separated aqueous layer was extracted with EtOAc. The combined organic layers were dried over sodium sulfate, filtered off and concentrated under reduced pressure. The residue was purified by column chromatography [silica gel, 24 g, EtOAc/heptane=10/90 to 50/50] providing tert-butyl 5′-chloro-5-fluoro-6-(((4-methoxytetrahydro-2H-pyran-4-yl)methyl)amino)-2,4′-bipyridinyl-2′-ylcarbamate (35 mg). LCMS (m/z): 467.1 [M+H]+; Rt=1.13 min.

### Step 2: Preparation of 5′-chloro-5-fluoro-N6-((tetrahydro-2H-pyran-4-yl)methyl)-2,4′-bipyridine-2′,6-diamine

A mixture of tert-butyl 5′-chloro-5-fluoro-6-(((4-methoxytetrahydro-2H-pyran-4-yl)methyl)amino)-2,4′-bipyridinyl-2′-ylcarbamate (35 mg, 0.075 mmol), trifluoroacetic acid (1 mL, 13 mmol) in dichloromethane (1.5 mL) was stirred at room temperature for 1 hr. The mixture was concentrated to dryness under reduced pressure. To the residue was added water (5 mL) and sodium carbonate (200 mg). The mixture was sonicated for 5 min and extracted with EtOAc (2×20 mL). The combined organic layers were washed with water (3×5 mL), dried over sodium sulfate and concentrated under reduced pressure providing crude 5′-chloro-5-fluoro-N6-((tetrahydro-2H-pyran-4-yl)methyl)-2,4′-bipyridine-2′,6-diamine (27 mg), which was directly used in the next reaction without further purification. LCMS (m/z): 367.0 [M+H]+; Rt=0.62 min.

### Synthesis of 6-(((6,6-dimethyl-1,4-dioxan-2-yl)methyl)amino)-5-fluoropyridin-2-yl trifluoromethanesulfonate

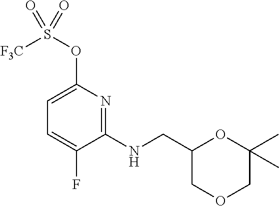

### Step 1: Preparation of 6-(iodomethyl)-2,2-dimethyl-1,4-dioxane

To a solution of 1-(allyloxy)-2-methylpropan-2-ol (5.0 g, 38 mmol) in acetonitrile (400 mL) was added sodium bicarbonate (19.5 g, 77 mmol) and the mixture was cooled to 0° C. Iodine (11.7 g, 46.1 mmol) was added and the reaction mixture was allowed to warm up to room temperature and stirred overnight. To the mixture was added triethylamine (6.42 mL, 46.1 mmol) and additional iodine (7.8 g, 30.7 mmol) and stirring was continued for additional 5 hrs at 0° C. To the mixture was added potassium carbonate (6.37 g, 46.1 mmol) and the suspension was stirred at room temperature for ˜3 days. The reaction mixture was diluted with saturated aqueous sodium thiosulfate solution (200 mL) and EtOAc (300 mL). The separated aqueous layer was extracted with EtOAc (2×) and the combined organic layers were dried over sodium sulfate, filtered off and concentrated under reduced pressure. The residue was purified by column chromatography [silica gel, EtOAc/hexane=10/100 to 10/40] providing 6-(iodomethyl)-2,2-dimethyl-1,4-dioxane as a yellow oil (2.07 g). ¹H NMR (400 MHz, chloroform-d) δ [ppm]: 4.01 (dd, J=11.2, 2.8 Hz, 1H), 3.81-3.88 (m, 1H), 3.44 (d, J=11.2 Hz, 1H), 3.22 (dd, J=11.6, 0.8 Hz, 1H), 2.97-3.13 (m, 3H), 1.33 (s, 3H), 1.14 (s, 3H). 1-(Allyloxy)-2-methylpropan-2-ol (1.63 g) was recovered.

### Step 2: Preparation of 6-(azidomethyl)-2,2-dimethyl-1,4-dioxane

To a solution of 6-(iodomethyl)-2,2-dimethyl-1,4-dioxane (1.80 g, 7.03 mmol) in anhydrous DMF (9 mL) was added sodium azide (0.685 g, 10.5 mmol) and the suspension was heated at 80° C. for 2.5 hrs. The mixture was diluted with water (30 mL) and EtOAc (30 mL). The separated organic layer was washed with water (3×). The aqueous layers were combined and extracted with EtOAc (1×). The combined organic layers, dried over sodium sulfate, filtered off and concentrated under reduced pressure. The residue was purified by column chromatography [silica gel, EtOAc/hexane=10/40 to 20/40] providing 6-(azidomethyl)-2,2-dimethyl-1,4-dioxane (0.93 g) as a colorless oil. 1H NMR (400 MHz, chloroform-d) δ [ppm]: 4.00-4.06 (m, 1H), 3.75 (ddd, J=11.2, 2.4, 0.4 Hz, 1H), 3.49 (d, J=11.2 Hz, 1H), 3.14-3.29 (m, 4H), 1.35 (s, 3H), 1.14 (s 3 H).

### Step 3: Preparation of (6,6-dimethyl-1,4-dioxan-2-yl)methanamine

To a solution of 6-(azidomethyl)-2,2-dimethyl-1,4-dioxane (502 mg, 2.93 mmol) in anhydrous tetrahydrofuran (15 mL) was added slowly a solution of lithium aluminumhydride (1M in tetrahydrofuran, 3.81 mL) 0° C. and the mixture was stirred at 0° C. for 1 hr and at room temperature for 0.5 hr. The reaction mixture was cooled to 0° C. and sodium sulfate decahydrate (excess) was slowly added and the suspension was vigorously stirred overnight. The suspension was filtered through cotton and the filtrate was concentrated under reduced pressure providing crude (6,6-dimethyl-1,4-dioxan-2-yl)methanamine (410 mg) as a colorless oil, which was directly used in the next step without purification. LCMS (m/z): 146.1 [M+H]+; Rt=0.42 min.

### Step 4: Preparation of N-((6,6-dimethyl-1,4-dioxan-2-yl)methyl)-3,6-difluoropyridin-2-amine

A mixture of 2,3,6-trifluoropyridine (282 mg, 2.12 mmol), 6,6-dimethyl-1,4-dioxan-2-yl)methanamine (280 mg, 1.93 mmol) and triethylamine (0.806 mL, 5.79 mmol) in acetonitrile (6 mL) was heated overnight at 70° C. The solvent removed under reduced pressure and the residue was purified by column chromatography [silica gel, EtOAc/hexane=20/80 to 50/50] providing N-((6,6-dimethyl-1,4-dioxan-2-yl)methyl)-3,6-difluoropyridin-2-amine (280 mg) as a colorless solid. LCMS (m/z): 259.1 [M+H]+; Rt=0.89 min.

### Step 5: Preparation of 6-(benzyloxy)-3-fluoro-N-((4-methyltetrahydro-2H-pyran-4-yl)methyl)pyridin-2-amine

To a solution of benzyl alcohol (0.542 mL, 5.21 mmol) in anhydrous DMF (4 mL) under argon was added carefully sodium hydride (60 wt. % in mineral oil, 208 mg, 5.21 mmol). The mixture was stirred at room temperature for 0.5 hr and a solution of N-((6,6-dimethyl-1,4-dioxan-2-yl)methyl)-3,6-difluoropyridin-2-amine (269 mg, 1.04 mmol) in DMF (3 mL) was added. Stirring was continued at 90° C. for 6 hrs. The reaction mixture was allowed to cool to room temperature, was diluted with EtOAc and washed with water (3×). The combined aqueous layers were extracted with EtOAc (1×). The combined organic layers were dried over sodium sulfate, filtered off and concentrated under reduced pressure. The residue was purified by column chromatography [silica gel, EtOAc/hexane=0/100 to 30/60] providing 6-(benzyloxy)-3-fluoro-N-((4-methyltetrahydro-2H-pyran-4-yl)methyl)pyridin-2-amine (335 mg) as a colorless solid. LCMS (m/z): 347.3 [M+H]+; Rt=1.20 min.

### Step 6: Preparation of 6-(((6,6-dimethyl-1,4-dioxan-2-yl)methyl)amino)-5-fluoropyridin-2-ol

To a solution of 6-(benzyloxy)-3-fluoro-N-((4-methyltetrahydro-2H-pyran-4-yl)methyl)pyridin-2-amine (334 mg, 0.964 mmol) in MeOH (8 mL) was added Pd/C (5 wt. %, water 50 wt. %, 103 mg). The mixture was stirred under hydrogen atmosphere (˜1 atm, balloon) overnight. The reaction mixture was filtered through a pad of celites and the filtrate was concentrated under reduced pressure The residue was purified by column chromatography [silica gel, EtOAc/hexane=0/100 to 50/50] providing 6-(((6,6-dimethyl-1,4-dioxan-2-yl)methyl)amino)-5-fluoropyridin-2-ol as a pink solid (155 mg). LCMS (m/z): 257.1 [M+H]+; Rt=0.53 min.

### Step 7: Preparation of 6-(((6,6-dimethyl-1,4-dioxan-2-yl)methyl)amino)-5-fluoropyridin-2-yl trifluoromethanesulfonate

To a solution of 6-(((6,6-dimethyl-1,4-dioxan-2-yl)methyl)amino)-5-fluoropyridin-2-ol (154 mg, 0.601 mmol) and triethylamine (0.126 mL, 0.901 mmol) in dichloromethane (10 mL) was added slowly trifluoromethanesulfonic anhydride (0.112 mL, 0.661 mmol) at 0° C. The mixture was stirred for 3 hrs at 0° C. The reaction mixture was diluted with saturated aqueous sodium carbonate solution and the separated aqueous layer was extracted with dichloromethane (2×). The combined organic layers were dried over sodium sulfate, filtered off and concentrated under reduced pressure providing crude 6-(((6,6-dimethyl-1,4-dioxan-2-yl)methyl)amino)-5-fluoropyridin-2-yl trifluoromethanesulfonate (230 mg) as a light yellow oil, which was directly used in the next step without purification. LCMS (m/z): 389.0 [M+H]+; Rt=1.08 min.

### Synthesis of 5′-chloro-N6-((6,6-dimethyl-1,4-dioxan-2-yl)methyl)-5-fluoro-2,4′-bipyridine-2′,6-diamine

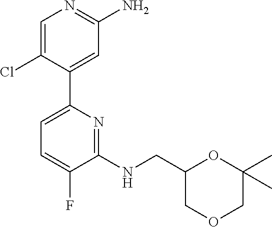

### Step 1: Preparation of 5′-chloro-N-((6,6-dimethyl-1,4-dioxan-2-yl)methyl)-2′,5-difluoro-2,4′-bipyridin-6-amine

A mixture of 6-((6,6-dimethyl-1,4-dioxan-2-yl)methylamino)-5-fluoropyridin-2-yl trifluoromethanesulfonate (230 mg, 0.592 mmol), 5-chloro-2-fluoropyridin-4-ylboronic acid (208 mg, 1.18 mmol), PdCl₂(dppf) CH₂Cl₂ adduct (48 mg, 0.059 mmol) and sodium carbonate (251 mg, 2.37 mmol) in DME (3 mL) and water (1.5 mL) was heated in a sealed tube at 110° C. for 25 min in a microwave reactor. The mixture was diluted with water and extracted with EtOAc. The combined organic layers were dried over sodium sulfate and concentrated under reduced pressure The residue was purified by column chromatography [silica gel, EtOAc/hexane=0/100 to 10/20] providing 5′-chloro-N-((6,6-dimethyl-1,4-dioxan-2-yl)methyl)-2′,5-difluoro-2,4′-bipyridin-6-amine as a colorless solid (177 mg). LCMS (m/z): 370.1 [M+H]+; Rt=1.11 min.

### Step 2: Preparation of 5′-chloro-N6-((6,6-dimethyl-1,4-dioxan-2-yl)methyl)-5-fluoro-2,4′-bipyridine-2′,6-diamine

A mixture of 5′-chloro-N-((6,6-dimethyl-1,4-dioxan-2-yl)methyl)-2′,5-difluoro-2,4′-bipyridin-6-amine (177 mg, 0.479 mmol) and aqueous ammonium hydroxide solution (28 wt. %, 1.5 mL) in DMSO (1 mL) was heated in a steel bomb at 125° C. for ˜18 hrs. The mixture was allowed to cool to room temperature and was diluted with EtOAc. The mixture was washed with water (3×) and the combined aqueous layers were extracted with EtOAc (1×). The combined organic layers were dried over sodium sulfate, filtered off and concentrated under reduced pressure. The residue was purified by column chromatography [silica gel, EtOAc/hexane=0/100 to 67/33]. Fractions were combined and concentrated under reduced pressure providing 5′-chloro-N6-((6,6-dimethyl-1,4-dioxan-2-yl)methyl)-5-fluoro-2,4′-bipyridine-2′,6-diamine (141 mg) as a colorless foam. LCMS (m/z): 367.0 [M+H]+; Rt=0.67 min.

### Synthesis of 6-((((5,5-dimethyl-1,4-dioxan-2-yl)methyl)amino)-5-fluoropyridin-2-yl trifluoromethanesulfonate

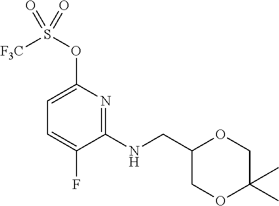

### Step 1: Preparation of 5-(iodomethyl)-2,2-dimethyl-1,4-dioxane

To a solution of 2-(allyloxy)-2-methylpropan-1-ol (5.0 g, 38.4 mmol) in acetonitrile (350 mL) was added sodium bicarbonate (9.68 g, 115 mmol) and the mixture was cooled to 0° C. Iodine (29.2 g, 115 mmol) was added and the reaction mixture was allowed to warm up to room temperature and stirred for 6 hrs. The reaction mixture was diluted with saturated aqueous sodium thiosulfate solution and concentrated under reduced pressure removing most of the organic solvent. The residue was extracted with EtOAc (2×) and the combined organic layers were dried over sodium sulfate, filtered off and concentrated under reduced pressure. The residue was purified by column chromatography [silica gel, EtOAc/hexane=10/100 to 10/40] providing 6-(iodomethyl)-2,2-dimethyl-1,4-dioxane as a colorless oil (7.04 g). ¹H NMR (400 MHz, chloroform-d) δ [ppm]: 3.70-3.73 (m, 1H), 3.57-3.64 (m, 2H), 3.43-3.50 (m, 2H), 3.13-3.15 (m, 2H), 1.32 (s, 3H), 1.13 (s, 3H).

### Step 2: Preparation of 5-(azidomethyl)-2,2-dimethyl-1,4-dioxane

To a solution of 5-(iodomethyl)-2,2-dimethyl-1,4-dioxane (2.58 g, 10.1 mmol) in anhydrous DMF (13 mL) was added sodium azide (0.982 g, 15.1 mmol) and the suspension was heated at 80° C. for 2.5 hrs. The mixture was diluted with water (40 mL) and EtOAc (40 mL). The separated organic layer was washed with water (3×). The aqueous layers were combined and extracted with EtOAc (1×). The combined organic layers, dried over sodium sulfate, filtered off and concentrated under reduced pressure. The residue was purified by column chromatography [silica gel, EtOAc/hexane=10/40 to 50/50] providing 6-(azidomethyl)-2,2-dimethyl-1,4-dioxane (1.61 g) as a colorless oil. NMR (400 MHz, chloroform-d) δ [ppm]: 3.63-3.72 (m, 2H), 3.52-3.59 (m, 2H), 3.42 (d, J=11.6 Hz, 1H), 3.29 (d, J=4.4 Hz, 2H), 1.33 (s, 3H), 1.13 (s, 3H).

### Step 3: Preparation of (5,5-dimethyl-1,4-dioxan-2-yl)methanamine

To a solution of 5-(azidomethyl)-2,2-dimethyl-1,4-dioxane (810 mg, 4.73 mmol) in anhydrous tetrahydrofuran (20 mL) was added slowly a solution of lithium aluminumhydride (1.0 M tetrahydrofuran, 6.2 mL) 0° C. and the mixture was stirred at 0° C. for 1 hr and at room temperature for 0.5 hr. The reaction mixture was cooled to 0° C. and sodium sulfate decahydrate (excess) was slowly added and the suspension was vigorously stirred overnight. The suspension was filtered through cotton and the filtrate was concentrated under reduced pressure providing crude (5,5-dimethyl-1,4-dioxan-2-yl)methanamine (673 mg) as a colorless oil, which was directly used in the next step without purification. LCMS (m/z): 146.1 [M+H]+; Rt=0.42 min.

### Step 4: Preparation of N-((5,5-dimethyl-1,4-dioxan-2-yl)methyl)-3,6-difluoropyridin-2-amine

A mixture of 2,3,6-trifluoropyridine (385 mg, 2.89 mmol), (5,5-dimethyl-1,4-dioxan-2-yl)methanamine (382 mg, 2.63 mmol) and triethylamine (1.10 mL, 7.89 mmol) in acetonitrile (8 mL) was heated at 70° C. overnight. The solvent removed under reduced pressure and the residue was purified by column chromatography [silica gel, EtOAc/hexane=20/80 to 50/50] providing N-((5,5-dimethyl-1,4-dioxan-2-yl)methyl)-3,6-difluoropyridin-2-amine (354 mg) as a colorless solid. LCMS (m/z): 259.1 [M+H]+; Rt=0.86 min.

### Step 5: Preparation of 6-(benzyloxy)-N-((5,5-dimethyl-1,4-dioxan-2-yl)methyl)-3-fluoropyridin-2-amine

To a solution of benzyl alcohol (0.705 mL, 6.78 mmol) in anhydrous DMF (5 mL) under argon was carefully added sodium hydride (60 wt. % in mineral oil, 2711 mg, 6.78 mmol). The mixture was stirred at room temperature for 0.5 hr and a solution of N-((5,5-dimethyl-1,4-dioxan-2-yl)methyl)-3,6-difluoropyridin-2-amine (350 mg, 1.36 mmol) in DMF (3 mL) was added. Stirring was continued at 90° C. for 6 hrs. The reaction mixture was allowed to cool to room temperature, was diluted with EtOAc and washed with water (3×). The combined aqueous layers were extracted with EtOAc (1×). The combined organic layers were dried over sodium sulfate, filtered off and concentrated under reduced pressure. The residue was purified by column chromatography [silica gel, EtOAc/hexane=00/100 to 30/60] providing 6-(benzyloxy)-N-((5,5-dimethyl-1,4-dioxan-2-yl)methyl)-3-fluoropyridin-2-amine (435 mg) as a colorless solid. LCMS (m/z): 347.3 [M+H]+; Rt=1.19 min.

### Step 6: Preparation of 6-(((5,5-dimethyl-1,4-dioxan-2-yl)methyl)amino)-5-fluoropyridin-2-ol

To a solution of 6-(benzyloxy)-N-((5,5-dimethyl-1,4-dioxan-2-yl)methyl)-3-fluoropyridin-2-amine (435 mg, 1.26 mmol) in MeOH (10 mL) was added Pd/C (5 wt. %, water 50 wt. %, 134 mg). The mixture was stirred under hydrogen atmosphere (˜1 atm, balloon pressure) overnight. The reaction mixture was filtered through a pad of celites and the filtrate was concentrated under reduced pressure The residue was purified by column chromatography [silica gel, EtOAc/hexane=00/100 to 50/50] providing 6-(((5,5-dimethyl-1,4-dioxan-2-yl)methyl)amino)-5-fluoropyridin-2-ol as a pink solid (156 mg). LCMS (m/z): 257.1 [M+H]+; Rt=0.54 min.

### Step 7: Preparation of 6-(((5,5-dimethyl-1,4-dioxan-2-yl)methyl)amino)-5-fluoropyridin-2-yl trifluoromethanesulfonate

To a solution of 6-(((5,5-dimethyl-1,4-dioxan-2-yl)methyl)amino)-5-fluoropyridin-2-ol (153 mg, 0.597 mmol) and triethylamine (0.125 mL, 0.896 mmol) in dichloromethane (10 mL) was added slowly trifluoromethanesulfonic anhydride (0.111 mL, 0.657 mmol) at 0° C. The mixture was stirred for 3 hrs at 0° C. The reaction mixture was diluted with saturated aqueous sodium carbonate solution and the separated aqueous layer was extracted with dichloromethane (2×). The combined organic layers were dried over sodium sulfate, filtered off and concentrated under reduced pressure providing crude 6-(((5,5-dimethyl-1,4-dioxan-2-yl)methyl)amino)-5-fluoropyridin-2-yl trifluoromethanesulfonate (231 mg) as a light yellow oil, which was directly used in the next step without purification. LCMS (m/z): 389.0 [M+H]+; Rt=1.07 min.

### Synthesis of 5′-chloro-N6-((5,5-dimethyl-1,4-dioxan-2-yl)methyl)-5-fluoro-2,4′-bipyridine-2′,6-diamine

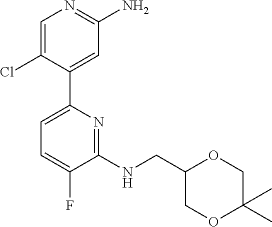

### Step 1: Preparation of 5′-chloro-N-((5,5-dimethyl-1,4-dioxan-2-yl)methyl)-2′,5-difluoro-2,4′-bipyridin-6-amine

A mixture of 6-((5,5-dimethyl-1,4-dioxan-2-yl)methylamino)-5-fluoropyridin-2-yl trifluoromethanesulfonate (230 mg, 0.592 mmol), 5-chloro-2-fluoropyridin-4-ylboronic acid (208 mg, 1.18 mmol), PdCl₂(dppf) CH₂Cl₂ adduct (48 mg, 0.059 mmol) and sodium carbonate (251 mg, 2.37 mmol) in DME (3 mL) and water (1.5 mL) was heated in a sealed tube at 110° C. for 25 min in a microwave reactor. The mixture was diluted with water and extracted with EtOAc. The combined organic layers were dried over sodium sulfate and concentrated under reduced pressure The residue was purified by column chromatography [silica gel, EtOAc/hexane=0/100 to 10/20] providing 5′-chloro-N-((5,5-dimethyl-1,4-dioxan-2-yl)methyl)-2′,5-difluoro-2,4′-bipyridin-6-amine as a colorless solid (164 mg). LCMS (m/z): 370.1 [M+H]+; Rt=1.09 min.

### Step 2: Preparation of 5′-chloro-N6-((5,5-dimethyl-1,4-dioxan-2-yl)methyl)-5-fluoro-2,4′-bipyridine-2′,6-diamine

A mixture of 5′-chloro-N-((5,5-dimethyl-1,4-dioxan-2-yl)methyl)-2′,5-difluoro-2,4′-bipyridin-6-amine (164 mg, 0.444 mmol), and ammonium hydroxide aqueous solution (28 wt. % in water, 1.5 mL) in DMSO (1 mL) was heated in a sealed vial at 125° C. for ˜18 hrs. The mixture was allowed to cool to room temperature and was diluted with EtOAc. The mixture was washed with water (3×) and the combined aqueous layers were extracted with EtOAc (1×). The combined organic layers were dried over sodium sulfate, filtered off and concentrated under reduced pressure. The residue was purified by column chromatography [silica gel, EtOAc/hexane=0/100 to 67/33]. Fractions were combined and concentrated under reduced pressure providing 5′-chloro-N6-((5,5-dimethyl-1,4-dioxan-2-yl)methyl)-5-fluoro-2,4′-bipyridine-2′,6-diamine (145 mg) as a colorless foam. LCMS (m/z): 367.0 [M+H]+; Rt=0.66 min.

### Synthesis of (6-bromo-pyridin-2-yl)-(1′,1′-dioxo-hexahydro-1-thiopyran-4-yl-methyl)-amine

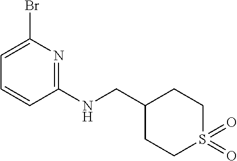

### Step 1: Preparation of toluene-4-sulfonic acid 1′,1′-dioxo-hexahydro-1-thiopyran-4-yl-methyl ester

A mixture of (1′,1′-dioxo-hexahydro-1-thiopyran-4-yl)-methanol (2.5 g, 15.22 mmol) [Organic Process Research & Development 2008, 12, 892-895.], pyridine (25 mL) and tosyl-Cl (2.90 g, 15.22 mmol) was stirred for 18 hrs at 50° C. The reaction mixture was concentrated under reduced pressure. The residue was purified by column chromatography [silica gel, EtOAc/hexane=0/100 to 70/30]. Fractions were combined and concentrated under reduced pressure providing toluene-4-sulfonic acid 1′,1′-dioxo-hexahydro-1-thiopyran-4-yl-methyl ester (3.78 g). LCMS (m/z): 319.0 [M+H]+; Rt=0.71 min.

### Step 2: Preparation of (6-bromo-pyridin-2-yl)-(1′,1′-dioxo-hexahydro-1-thiopyran-4-yl-methyl)-amine

To a mixture of 2-amino-6-bromopyridine (0.543 g, 3.14 mmol) and potassium carbonate (0.868 g, 6.28 mmol) in DMF (6 mL) was added toluene-4-sulfonic acid 1′,1′-dioxo-hexahydro-1-thiopyran-4-yl-methyl ester (1 g, 3.14 mmol) followed by sodium hydride (0.126 g, 3.14 mmol). The mixture was stirred in a sealed tube at 60° C. for 18 hrs. The reaction mixture was diluted with EtOAc, washed with water, saturated aqueous sodium bicarbonate solution and brine. The organic layer was dried over sodium sulfate, filtered off and concentrated under reduced pressure. The crude solid was purified by column chromatography [silica gel, EtOAc/hexane=0/100 to 50/50]. Fractions were combined and concentrated under reduced pressure providing (6-bromo-pyridin-2-yl)-(1′,1′-dioxo-hexahydro-1-thiopyran-4-yl-methyl)-amine (270 mg). LCMS (m/z): 318.8 [M+H]+; Rt=0.73 min.

### Synthesis of 5′-chloro-N6-(((1,1-dioxo)-tetrahydro-2H-1-thiopyran-4-yl)methyl)-[2,4′]bipyridinyl-6,2′-diamine

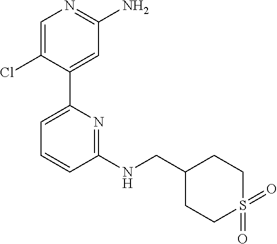

### Step 1: Preparation of (5′-chloro-2′-fluoro-[2,4′]bipyridinyl-6-yl)-(1′,1′-dioxo-hexahydro-1-thiopyran-4-ylmethyl)-amine

To (6-bromo-pyridin-2-yl)-(1,1-dioxo-hexahydro-1-thiopyran-4-yl-methyl)-amine (270 mg, 0.846 mmol) was added 5-chloro-2-fluoropyridin-4-ylboronic acid (297 mg, 1.692 mmol), PdCl₂(dppf) CH₂Cl₂ adduct (55.3 mg, 0.068 mmol), DME (5 mL) and 2M aqueous sodium carbonate solution (1.1 mL, 2.199 mmol). The reaction mixture was stirred at 75° C. for 18 hrs. The reaction mixture was concentrated to dryness under reduced pressure, diluted with EtOAc and washed with saturated aqueous sodium bicarbonate solution and brine. The organic layer was dried over sodium sulfate, filtered off and concentrated under reduced pressure. The residue was purified by column chromatography [silica gel, EtOAc/hexane=0/100 to 50/50] to yield (5′-chloro-2′-fluoro-[2,4′]bipyridinyl-6-yl)-(1′,1′-dioxo-hexahydro-1-thiopyran-4-ylmethyl)-amine (210 mg). LCMS (m/z): 370.0 [M+H]+; Rt=0.62 min.

### Step 2: Preparation of 5′-chloro-N6-(((1,1-dioxo)-tetrahydro-2H-1-thiopyran-4-yl)methyl)-[2,4′]bipyridinyl-6,2′-diamine

A mixture of (5′-chloro-2′-fluoro-[2,4′]bipyridinyl-6-yl)-(1′,1′-dioxo-hexahydro-1-thiopyran-4-ylmethyl)-amine (280 mg, 0.757 mmol) and ammonium hydroxide (aqueous solution 30-35 wt. %, 3 mL) in DMSO (3 mL) in a sealed tube and under argon was heated at 100° C. for 72 hrs. The reaction mixture was concentrated to dryness. The crude product was purified by column chromatography [silica gel, EtOAc/hexane]. Fractions were combined and concentrated under reduced pressure providing 5′-chloro-N6-(((1,1-dioxo)-tetrahydro-2H-1-thiopyran-4-yl)methyl)-[2,4′]bipyridinyl-6,2′-diamine (95 mg). LCMS (m/z): 367.0 [M+H]+; Rt=0.40 min.

### Synthesis of (R)-tert-butyl 3-(5-chloro-4-iodopyridin-2-ylcarbamoyl)piperidine-1-carboxylate

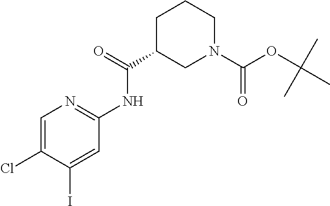

### Step 1: Preparation of 5-chloro-4-iodopyridin-2-amine

A mixture of 5-chloro-2-fluoro-4-iodopyridine (4.120 g, 16.00 mmol) and aqueous ammonium hydroxide solution (32 wt. %, 70 mL) in DMSO (70 mL) was heated in a sealed steel bomb at 90° C. for 18 hrs. The mixture was cooled to room temperature and diluted with EtOAc (450 mL). The mixture was washed with water (3×) and brine (1×), dried over sodium sulfate, filtered off and concentrate under reduced pressure providing crude 5-chloro-4-iodopyridin-2-amine (3.97 g), which was directly used in the next step without further purification. LCMS (m/z): 254.9 [M+H]+; Rt=0.43 min.

### Step 2: Preparation of (R)-tert-butyl 3-(5-chloro-4-iodopyridin-2-ylcarbamoyl)piperidine-1-carboxylate

To a solution of (R)-1-(tert-butoxycarbonyl)piperidine-3-carboxylic acid (1.081 g, 4.72 mmol) in dichloromethane (6 mL) at 0° C. was added 1-chloro-N,N,2-trimethylprop-1-en-1-amine (0.735 g, 5.50 mmol). The mixture was stirred at room temperature for 30 min and added to a solution of 5-chloro-4-iodopyridin-2-amine (1.00 g, 3.93 mmol) and pyridine (0.445 mL, 5.50 mmol) in tetrahydrofuran (6 mL). The reaction mixture was stirred at room temperature for 2 hrs. The mixture was diluted with EtOAc (350 mL) and washed with saturated aqueous sodium bicarbonate solution (1×), water (2×), brine (1×), dried over sodium sulfate, filtered off and concentrated under reduced pressure. The residue was purified by column chromatography [silica gel, 40 g, EtOAc/heptane=0/100 to 75/25] providing (R)-tert-butyl 3-(5-chloro-4-iodopyridin-2-ylcarbamoyl)piperidine-1-carboxylate (1.80 g). LCMS (m/z): 466.0 [M+H]+; Rt=1.06 min.

### Synthesis of (R)-tert-butyl 3-(5′-chloro-6-fluoro-2,4′-bipyridinyl-2′-ylcarbamoyl)piperidine-1-carboxylate

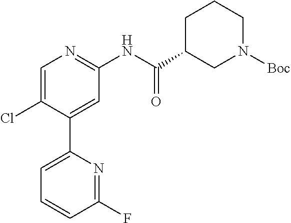

### Step 1: Preparation of 2-fluoro-6-(4,4,5,5-tetramethyl-1,3,2-dioxaborolan-2-yl)pyridine

A mixture of 2-bromo-6-fluoropyridine (1.056 g, 6 mmol), 4,4,4′,4′,5,5,5′,5′-octamethyl-2,2′-bi(1,3,2-dioxaborolane) (1.60 g, 6.30 mmol), PdCl₂(dppf) CH₂Cl₂ adduct (0.294 g, 0.360 mmol) and potassium acetate (1.767 g, 18.00 mmol) in dioxane (12 mL) was stirred at 100° C. for 18 hrs. The reaction mixture was cooled to room temperature, diluted with EtOAc (40 mL), filtered and concentrated under reduced pressure. The crude material of 2-fluoro-6-(4,4,5,5-tetramethyl-1,3,2-dioxaborolan-2-yl)pyridine was directly used in the next step without further purification. LCMS (m/z): 142.0 [MS fragment]; Rt=0.35 min. [Note: LCMS shows only boronic acid fragment.]

### Step 2: Preparation of (R)-tert-butyl 3-(5′-chloro-6-fluoro-2,4′-bipyridinyl-2′-ylcarbamoyl)piperidine-1-carboxylate

To a mixture of (R)-tert-butyl 3-(5-chloro-4-iodopyridin-2-ylcarbamoyl)piperidine-1-carboxylate (1.050 g, 2.255 mmol), 2-fluoro-6-(4,4,5,5-tetramethyl-1,3,2-dioxaborolan-2-yl)pyridine (1.106 g, 4.96 mmol) and PdCl₂(dppf) CH₂Cl₂ adduct (0.184 g, 0.225 mmol) in DME (18 mL) was added 2M aqueous sodium carbonate solution (6.20 mL, 12.40 mmol). The reaction mixture was stirred at 95° C. for 90 min. The mixture was cooled to room temperature and diluted with EtOAc (20 mL) and MeOH (15 mL), filtered and concentrated under reduced pressure. The crude material was purified by column chromatography [silica gel, 40 g, EtOAc/heptane=10/90 to 40/60] providing (R)-tert-butyl 3-(5′-chloro-6-fluoro-2,4′-bipyridin-2′-ylcarbamoyl)piperidine-1-carboxylate (851 mg). LCMS (m/z): 435.1 [M+H]+; Rt=0.99 min.

### Synthesis of 1-(tert-butoxycarbonyl)-3-fluoropiperidine-3-carboxylic acid

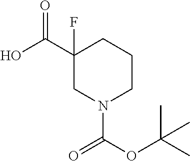

### Step 1: Preparation of 1-tert-butyl 3-methyl (3-fluoropiperidine)-1,3-dicarboxylate

To a solution of LDA [freshly prepared from BuLi (1.6M solution in hexanes, 5.14 mL, 8.22 mmol) and diisopropylamine (1.44 mL, 10.39 mmol) in tetrahydrofuran (6 mL) at 0° C.] was added dropwise a solution of 1-tert-butyl 3-methyl piperidine-1,3-dicarboxylate (2 g, 8.22 mmol) in tetrahydrofuran (8 mL) at 0° C. The solution was stirred at 0° C. for 30 min and then transferred to a 0° C. solution of N-fluorobenzenesulfonimide (3.24 g, 10.28 mmol) in tetrahydrofuran (12 mL). The reaction mixture was stirred at 0° C. for 15 min and then at room temperature for ˜20 hrs. The total solvent volume was reduced under reduced pressure to approximately one third and EtOAc was added. The mixture was washed with water, 0.1N aqueous hydrochloride solution, saturated aqueous sodium bicarbonate solution and brine. The organic phase was dried over sodium sulfate, filtered off and concentrated under reduced pressure. The crude was suspended in EtOAc and decanted. The filtrate was concentrated under reduced pressure and purified by column chromatography [silica gel, 80 g, EtOAc/heptane=0/100 to 50/50] providing 1-tert-butyl 3-methyl (3-fluoropiperidine)-1,3-dicarboxylate (775 mg) as a colorless liquid. LCMS (m/z): 262.1 [M+H]+, 206.1 [M+H, loss of t-Bu]+; Rt=0.86 min.

### Step 2: Preparation of 1-(tert-butoxycarbonyl)-3-fluoropiperidine-3-carboxylic acid

To a solution of 1-tert-butyl 3-methyl 3-fluoropiperidine-1,3-dicarboxylate (250 mg, 0.957 mmol) in MeOH (6 mL) was added slowly 2N aqueous sodium hydroxide solution (6 mL, 12.00 mmol) and the mixture was stirred for 2 hrs at room temperature. The reaction mixture was acidified with 1N aqueous hydrochloride solution and extracted with diethylether (3×). The combined organic layers were dried over sodium sulfate, filtered off and concentrated under reduced pressure providing crude 1-(tert-butoxycarbonyl)-3-fluoropiperidine-3-carboxylic acid (215 mg) as a white solid, The crude material was directly used in the next reaction without further purification. LCMS (m/z): 192.0 [M+H, loss of t-Bu]+; Rt=0.69 min.

### Synthesis of (3R,4S)-1-(benzyloxycarbonyl)-4-fluoropyrrolidine-3-carboxylic acid

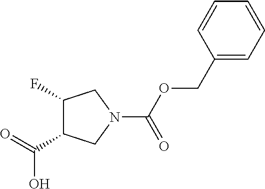

### Step 1: Preparation of (3S,4S)-benzyl 3-fluoro-4-vinylpyrrolidine-1-carboxylate

To a solution of (3R,4S)-benzyl 3-hydroxy-4-vinylpyrrolidine-1-carboxylate (5.0 g, 20.22 mmol) in (trifluoromethyl)benzene (84 mL) under argon was added diisopropylethylamine (53.0 mL, 303 mmol) and triethylamine trihydrofluoride (19.75 mL, 121 mmol). Perfluorobutanesulfonyl fluoride (PBSF) (9.09 mL, 50.5 mmol) was added slowly in five portions, each portion every in 30 min. The reaction mixture was stirred overnight. The organic solution was washed with 1N aqueous hydrochloride solution (2×), saturated aqueous sodium bicarbonate solution (2×) and water, dried over sodium sulfate, filtered off and concentrated under reduced pressure. The residue was purified by column chromatography [silica gel, 120 g, EtOAc/heptane=0/100 to 50/50] providing (3S,4S)-benzyl 3-fluoro-4-vinylpyrrolidine-1-carboxylate (3.8 g). LCMS (m/z): 250.0 [M+H]+; Rt=0.92 min.

### Step 2: Preparation of (3R,4S)-1-(benzyloxycarbonyl)-4-fluoropyrrolidine-3-carboxylic acid

A mixture of (3S,4S)-benzyl 3-fluoro-4-vinylpyrrolidine-1-carboxylate (3.8 g, 15.24 mmol), ruthenium trichloride (199 mg, 0.762 mmol), sodium periodate (13.04 g, 61.0 mmol) in carbontetrachloride (43.6 mL), water (65.3 mL) and acetonitrile (43.6 mL) was stirred overnight at room temperature. The reaction mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (3.05 g, 30.5 mmol) and 1N aqueous sulfuric acid solution (50 mL) were added. The resulting mixture was stirred at room temperature for 3 hrs. The reaction mixture was extracted with dichloromethane (2×100 mL). The combined organic layers were concentrated under reduced pressure and the residue was purified by column chromatography [silica gel] providing (3R,4S)-1-(benzyloxycarbonyl)-4-fluoropyrrolidine-3-carboxylic acid (2.9 g). LCMS (m/z): 268.0 [M+H]+; Rt=0.68 min.

### Synthesis of (3S,4S)-1-(benzyloxycarbonyl)-4-(tert-butyldiphenylsilyloxy)pyrrolidine-3-carboxylic acid

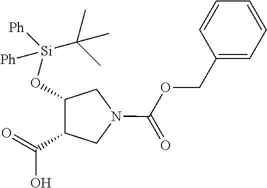

### Step 1: Preparation of (3S,4S)-benzyl 3-(4-methoxybenzoyloxy)-4-vinylpyrrolidine-1-carboxylate

A mixture of (3R,4S)-benzyl 3-hydroxy-4-vinylpyrrolidine-1-carboxylate (2.25 g, 9.10 mmol), p-anisic acid (1.66 g, 10.92 mmol), N1,N1,N2,N2-tetramethyldiazene-1,2-dicarboxamide (2.350 g, 13.65 mmol), benzene (18.20 mL) and tributyl phosphine (3.37 mL, 13.65 mmol) was stirred in a closed vial at 60° C. for 2 hrs. The reaction mixture was cooled to ambient temperature, and diluted with EtOAc (100 mL). The mixture was washed with water, brine, dried over sodium sulfate, filtered off and concentrated under reduced pressure providing (3S,4S)-benzyl 3-(4-methoxybenzoyloxy)-4-vinylpyrrolidine-1-carboxylate (2.58 g), which was directly used in the next step without further purification. LCMS (m/z): 382.2 [M+H]+; Rt=1.08 min.

### Step 2: Preparation of (3S,4S)-benzyl 3-hydroxy-4-vinylpyrrolidine-1-carboxylate

To a solution of crude (3S,4S)-benzyl 3-(4-methoxybenzoyloxy)-4-vinylpyrrolidine-1-carboxylate (2.58 g) in tetrahydrofuran (30 mL) was added 1N aqueous sodium hydroxide solution (30 mL) and the mixture was stirred at 60° C. for 18 hrs. The reaction mixture was cooled to room temperature and diluted with EtOAc (100 mL). The mixture was washed with water, brine, dried over sodium sulfate, filtered off and concentrated under reduced pressure. The residue was purified by column chromatography [silica gel] providing (3S,4S)-benzyl 3-hydroxy-4-vinylpyrrolidine-1-carboxylate (1.8 g). LCMS (m/z): 248.1 [M+H]+; Rt=0.87 min.

### Step 3: Preparation of (3S,4S)-benzyl 3-(tert-butyldiphenylsilyloxy)-4-vinylpyrrolidine-1-carboxylate

To a solution of (3S,4S)-benzyl 3-hydroxy-4-vinylpyrrolidine-1-carboxylate (1.8 g, 7.28 mmol) in dichloromethane (14 mL) was added imidazole (0.842 g, 12.37 mmol) and tert-butylchlorodiphenylsilane (2.057 mL, 8.01 mmol). The reaction mixture was stirred at room temperature for 18 hrs and filtered through a thin layer of celite. The filtrate was washed with water and brine, dried over sodium sulfate, filtered off and concentrated under reduced pressure providing crude (3S,4S)-benzyl 3-(tert-butyldiphenylsilyloxy)-4-vinylpyrrolidine-1-carboxylate (2.4 g), which was directly used in the next step without further purification. LCMS (m/z): 486.2 [M+H]+; Rt=1.44 min.

### Step 4: Preparation of (3S,4S)-1-(benzyloxycarbonyl)-4-(tert-butyldiphenylsilyloxy)-pyrrolidine-3-carboxylic acid

A mixture of (3S,4S)-benzyl 3-(tert-butyldiphenylsilyloxy)-4-vinylpyrrolidine-1-carboxylate (3.9 g, 8.03 mmol), ruthenium trichloride (0.105 g, 0.401 mmol), sodium periodate (6.87 g, 32.1 mmol) in carbontetrachloride (11.5 mL), water (17.2 mL) and acetonitrile (11.5 mL) was stirred at overnight room temperature. The mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (1.606 g, 16.06 mmol), and 1N aqueous sulfuric acid solution (50 mL) were added. The mixture was stirred at room temperature for 3 hrs. The reaction mixture was extracted with dichloromethane (2×100 mL). The combined organic layers were concentrated under reduced pressure. The residue was purified by column chromatography [silica gel] providing (3S,4S)-1-(benzyloxycarbonyl)-4-(tert-butyldiphenylsilyloxy)pyrrolidine-3-carboxylic acid (2.5 g). LCMS (m/z): 504.1 [M+H]+; Rt=1.18 min.

### Synthesis of (3S,4R)-1-(benzyloxycarbonyl)-4-(tert-butyldiphenylsilyloxy)pyrrolidine-3-carboxylic acid

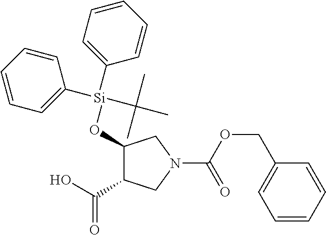

### Step 1: Preparation of benzyl 2,5-dihydro-1H-pyrrole-1-carboxylate

To a solution of 2,5-dihydro-1H-pyrrole (30 g, 434 mmol) in dioxane (1000 mL) was added CbzOSu (130 g, 521 mmol) and the mixture was stirred at room temperature for 18 hrs. The reaction mixture was concentrated to a volume of ˜300 mL and diluted with EtOAc (1000 mL). The organic layer was washed with water and brine, dried over sodium sulfate, filtered off and concentrated under reduced pressure. The residue was purified by column chromatography [silica gel] providing benzyl 2,5-dihydro-1H-pyrrole-1-carboxylate (80.0 g) as a colorless oil. Rf=0.6 (EtOAc/hexanes=30:70). ¹H NMR (400 MHz, chloroform-d) δ [ppm]: 7.32 (m, 5H), 5.80 (m, 2H), 5.77 (s, 2H), 4.22 (m, 4H). LCMS (m/z): 204.2 [M+H]+; Rt=0.86 min.

### Step 2: Preparation of benzyl 6-oxa-3-azabicyclo[3.1.0]hexane-3-carboxylate

To a solution of benzyl 2,5-dihydro-1H-pyrrole-1-carboxylate (33 g, 163 mmol) in dichloromethane (540 mL) was added MCPBA (77 wt. %, 44 g) and the reaction mixture was stirred at room temperature for 18 hrs. The mixture was diluted with saturated aqueous sodium carbonate solution (500 mL) and the resulting mixture was stirred at room temperature for 1 hr. The separated organic layer washed with water and brine, dried over sodium sulfate, filtered off and concentrated under reduced pressure. The residue was purified by column chromatography [silica gel] providing benzyl 6-oxa-3-azabicyclo[3.1.0]hexane-3-carboxylate (29.5 g) as a yellow oil. ¹H NMR (400 MHz, chloroform-d) δ [ppm]: 3.38 (dd, J=12.8, 6.0 Hz, 2H), 3.68 (d, J=3.6 Hz, 2H), 3.87 (dd, J=13.2, 19.6, 2 H), 5.11 (s, 2H), 7.33 (m, 5H). LCMS (m/z): 220.0 [M+H]+; Rt=0.69 min.

### Step 3: Preparation of trans-(±)-benzyl 3-hydroxy-4-vinylpyrrolidine-1-carboxylate

To a solution of benzyl 6-oxa-3-azabicyclo[3.1.0]hexane-3-carboxylate (28.5 g, 130 mmol) and CuBr.SMe₂ (26.7 g, 130 mmol) in anhydrous THF (260 mL) at −40° C. was slowly added vinyl magnesium bromide (1.0 M solution in THF, 520 mL). The reaction mixture was warmed up to −20° C. for 2 hrs. The mixture was quenched with saturated aqueous ammonium chloride solution (200 mL) and extracted with EtOAc (500 mL). The organic layer was washed with water and brine, dried over sodium sulfate, filtered off and concentrated under educed pressure. The residue was purified by column chromatography [silica gel] providing a racemic mixture of trans-(±)-benzyl 3-hydroxy-4-vinylpyrrolidine-1-carboxylate (15.5 g) as a yellow oil. Rf=0.2 (EtOAc/hexanes=30:70). ¹H NMR (400 MHz, chloroform-d) δ [ppm]: 2.71 (m, 1H), 3.28 (m, 2H), 3.72 (m, 2H), 4.11 (m, 1H), 5.14 (s, 2H), 5.16-5.23 (m, 2H), 5.69 (m, 1H), 7.33 (m, 5H). LCMS (m/z): 248.0 [M+H]+; Rt=0.78 min.

### Step 4: Resolution of (3S,4R)-benzyl 3-hydroxy-4-vinylpyrrolidine-1-carboxylate and (3R,4S)-benzyl 3-hydroxy-4-vinylpyrrolidine-1-carboxylate

Amount: 10 g dissolved in {n-hexane:ethanol:methanol}={8:2:1}; 200 mg/mL.

### Analytical Separation:

### Column: CHIRALPAK AD (20 um) 250×4.6 mm.

Solvent: n-heptane: ethanol: methanol=8:1:1.
Flow rate: 1.0 mL/min; detection: UV=220 nm.
Fraction 1: Retention time: 9.16 min.
Fraction 2: Retention time: 13.10 min.

### Preparative Separation:

Column: CHIRALPAK AD-prep (20 um) 5 cm×50 cm.
Solvent: n-heptane:ethanol:methanol=8:1:1.
Flow rate: 100 mL/min; injection per run: 1000 mg/5 mL; detection: UV=220 nm.
Fraction 1: (3S,4R)-benzyl 3-hydroxy-4-vinylpyrrolidine-1-carboxylate. Brownish liquid. Yield: 4530 mg; ee=99.5% (UV, 220 nm).
Fraction 2: (3R,4S)-benzyl 3-hydroxy-4-vinylpyrrolidine-1-carboxylate. Brownish liquid. Yield: 4117 mg; ee=99.5% (UV, 220 nm).

### Step 5: Preparation of (3R,4S)-benzyl 3-(tert-butyldiphenylsilyloxy)-4-vinylpyrrolidine-1-carboxylate

To a solution of (3R,4S)-benzyl 3-hydroxy-4-vinylpyrrolidine-1-carboxylate (3.0 g, 12.13 mmol) in dichloromethane (24 mL) was added imidazole (1.404 g, 20.62 mmol) and tert-butylchlorodiphenylsilane (3.43 mL, 13.34 mmol). The reaction mixture was stirred at room temperature for 18 hrs and filtered through a thin layer of celite. The filtrate was washed with water and brine, dried over sodium sulfate, filtered off and concentrated under reduced pressure providing crude (3R,4S)-benzyl 3-(tert-butyldiphenylsilyloxy)-4-vinylpyrrolidine-1-carboxylate (6.2 g), which was directly used in the next step without further purification. LCMS (m/z): 486.2 [M+H]+; Rt=1.46 min.

### Step 6: Preparation of (3S,4R)-1-(benzyloxycarbonyl)-4-(tert-butyldiphenylsilyloxy)pyrrolidine-3-carboxylic acid

A mixture of (3R,4S)-benzyl 3-(tert-butyldiphenylsilyloxy)-4-vinylpyrrolidine-1-carboxylate, ruthenium trichloride (0.167 g, 0.638 mmol), sodium periodate (10.92 g, 51.1 mmol) in carbontetrachloride (18.2 mL), water (27.4 mL) and acetonitrile (18.2 mL) was stirred overnight at room temperature. The mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (2.55 g, 25.5 mmol), and 1N aqueous sulfuric acid solution (50 mL) were added. The mixture was stirred at room temperature for 3 hrs. The reaction mixture was extracted with dichloromethane (2×100 mL). The combined organic layers were concentrated under reduced pressure. The residue was purified by column chromatography [silica gel] providing (3S,4R)-1-(benzyloxycarbonyl)-4-(tert-butyldiphenylsilyloxy)pyrrolidine-3-carboxylic acid (3.5 g). LCMS (m/z): 504.1 [M+H]+; Rt=1.26 min.

### Synthesis of (3R,5S)-1-(tert-butoxycarbonyl)-5-(methoxymethyl)pyrrolidine-3-carboxylic acid

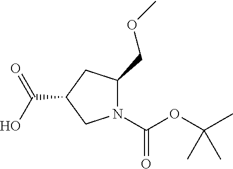

### Step 1: Preparation of (2S,4S)-4-methanesulfonyloxy-pyrrolidine-1,2-dicarboxylic acid 1-tert-butyl ester 2-methyl ester

A mixture of (2S,4S)-4-hydroxy-pyrrolidine-1,2-dicarboxylic acid 1-tert-butyl ester 2-methyl ester (5.0 g, 20.39 mmol), N,N-diisopropyl-N-ethylamine (3.16, 24.46 mmol) and methanesulfonyl chloride (2.8 g, 24.46 mmol) in dichloromethane (50 mL) was stirred at 23° C. for 18 hrs. The reaction mixture was concentrated under reduced pressure and the residue was purified by column chromatography [silica gel, 80 g, EtOAc/heptane=0/100 to 40/60] providing (2S,4S)-4-methanesulfonyloxy-pyrrolidine-1,2-dicarboxylic acid 1-tert-butyl ester 2-methyl ester (6.0 g). LCMS (m/z): 324.1 [M+H]+; Rt=0.69 min.

### Step 2: Preparation of (2S,4S)-tert-butyl 2-(hydroxymethyl)-4-(methylsulfonyloxy)pyrrolidine-1-carboxylate

To a solution of (2S,4S)-4-methanesulfonyloxy-pyrrolidine-1,2-dicarboxylic acid 1-tert-butyl ester 2-methyl ester (5.0 g) in tetrahydrofuran (31 mL) was added sodium borohydride (1.170 g, 30.9 mmol) and the mixture was heated to reflux for 3 hrs. The reaction mixture was allowed to cool to room temperature and was diluted with saturated aqueous ammonium chloride solution (5 mL) and EtOAc (100 mL). The mixture was washed with water, aqueous sodium bicarbonate solution and brine and concentrated under reduced pressure. The residue was purified by column chromatography [silica gel, 40 g, EtOAc/heptane=0/100 to 70/30] providing (2S,4S)-tert-butyl 2-(hydroxymethyl)-4-(methylsulfonyloxy)pyrrolidine-1-carboxylate (4.0 g). LCMS (m/z): 296.0 [M+H]+; Rt=0.59 min.

### Step 3: Preparation of (2S,4S)-tert-butyl 2-((tert-butyldiphenylsilyloxy)methyl)-4-(methylsulfonyloxy)pyrrolidine-1-carboxylate

To a solution of (2S,4S)-tert-butyl 2-(hydroxymethyl)-4-(methylsulfonyloxy)pyrrolidine-1-carboxylate (4.0 g, 16.18 mmol) in dichloromethane (32.4 mL) was added imidazole (1.872 g, 27.5 mmol) and tert-butylchlorodiphenylsilane (4.57 mL, 17.79 mmol). The reaction mixture was stirred at room temperature for 18 hrs and filtered through a thin layer of celite. The filtrate was washed with water and brine, dried over sodium sulfate, filtered off and concentrated under reduced pressure. The residue was purified by column chromatography [silica gel, EtOAc/heptane=0/100 to 40/60] providing (2S,4S)-tert-butyl 2-((tert-butyldiphenylsilyloxy)methyl)-4-(methylsulfonyloxy)pyrrolidine-1-carboxylate (6.0 g). LCMS (m/z): 534.5 [M+H]+; Rt=1.33 min.

### Step 4: Preparation of (2S,4R)-tert-butyl 2-((tert-butyldiphenylsilyloxy)methyl)-4-cyanopyrrolidine-1-carboxylate

To a solution of (2S,4S)-tert-butyl 2-((tert-butyldiphenylsilyloxy)methyl)-4-methylsulfonyloxy)pyrrolidine-1-carboxylate (6 g, 11.24 mmol) in DMF (50 mL) was added tetrabutylammonium cyanide (3.62 g, 13.49 mmol) and the mixture was stirred at 60° C. for 18 hrs. The reaction mixture was diluted with EtOAc (50 mL) and washed with water and brine. The organic layer was dried over sodium sulfate for ˜18 hrs, filtered off and concentrated under reduced pressure. The residue was purified by column chromatography [silica gel, EtOAc/heptane=0/100 to 50/50] providing (2S,4R)-tert-butyl 2-((tert-butyldiphenylsilyloxy)methyl)-4-cyanopyrrolidine-1-carboxylate (3.8 g). LCMS (m/z): 465.2 [M+H]+; Rt=1.37 min.

### Step 5: Preparation of (2S,4R)-tert-butyl 4-cyano-(2-hydroxymethyl)pyrrolidine-1-carboxylate

To a solution of (2S,4R)-tert-butyl 2-((tert-butyldiphenylsilyloxy)methyl)-4-cyanopyrrolidine-1-carboxylate (3.8 g, 8.18 mmol) in tetrahydrofuran (30 mL) was added tetrabutylammonium fluoride (2.57 g, 9.81 mmol) and the mixture was stirred at 23° C. for 3 hrs. The reaction mixture was concentrated under reduced pressure and the residue was dissolved in EtOAc (50 mL). The organic solution was washed with water, brine, dried over sodium sulfate, filtered off and concentrated under reduced pressure. The residue was purified by column chromatography [silica gel] providing (2S,4R)-tert-butyl 4-cyano-(2-hydroxymethyl)pyrrolidine-1-carboxylate (1.7 g).

### Step 6: Preparation of (2S,4R)-tert-butyl 4-cyano-2-(methoxymethyl)pyrrolidine-1-carboxylate

To a solution of (2S,4R)-tert-butyl 4-cyano-2-(hydroxymethyl)pyrrolidine-1-carboxylate (850 mg, 3.76 mmol) in tetrahydrofuran (20 mL) was carefully added sodium hydride (60 wt. % in mineral oil, 184 mg, 4.51 mmol) and the mixture was stirred at room temperature for 30 min. To the mixture was added methyl iodide (0.470 mL, 7.51 mmol) and stirring was continued at room temperature for 3 hrs. The reaction mixture was diluted carefully with aqueous saturated ammonium chloride solution (50 mL) and EtOAc (100 mL). The organic layer was concentrated under reduced pressure and the residue was dissolved in EtOAc (100 mL). The mixture was washed with water (2×50 mL) and brine (2×100 mL), dried over sodium sulfate, filtered off and concentrated under reduced pressure. The residue was purified by column chromatography [silica gel, EtOAc/heptane=0/100 to 60/40] providing (2S,4R)-tert-butyl 4-cyano-2-(methoxymethyl)pyrrolidine-1-carboxylate (560 mg). LCMS (m/z): 241.2 [M+H]+; Rt=0.76 min.

### Step 7: Preparation of (3R,5S)-1-(tert-butoxycarbonyl)-5-(methoxymethyl)pyrrolidine-3-carboxylic acid

A mixture of (2S,4R)-tert-butyl 4-cyano-2-(methoxymethyl)pyrrolidine-1-carboxylate (600 mg, 2.497 mmol), 6N aqueous sodium hydroxide solution (13.73 mL, 82 mmol) and EtOH (15 mL) in a closed vial was stirred at 80° C. for 1 hr. The reaction mixture was allowed to cool to room temperature, acidified with 1N aqueous hydrochloride solution until pH˜5 and extracted with dichloromethane (3×100 mL). The combined organic layers were concentrated under reduced pressure and the residue was dissolved in EtOAc. The organic layer was washed with water, brine, dried over sodium sulfate filtered off and concentrated under reduced pressure. The residue was purified by column chromatography [silica gel] providing (3R,5S)-1-(tert-butoxycarbonyl)-5-(methoxymethyl)pyrrolidine-3-carboxylic acid (510 mg). LCMS (m/z): 260.2 [M+H]+; Rt=0.69 min. ¹H NMR (400 MHz, methanol-d) δ [ppm]: 1.46 (s, 9H) 2.10-2.20 (m, 2H) 3.15-3.26 (m, 1H) 3.34 (s, 3H) 3.44 (d, J=4.30 Hz, 2H) 3.47-3.60 (m, 2H) 3.94-4.05 (m, 1H).

### Synthesis of 4-(tert-butoxycarbonyl)-2-methylmorpholine-2-carboxylic acid

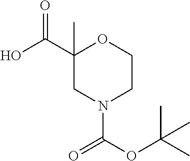

### Step 1: Preparation of 4-tert-butyl 2-methyl morpholine-2,4-dicarboxylate

To a solution of 4-(tert-butoxycarbonyl)morpholine-2-carboxylic acid (500 mg, 2.162 mmol) in MeOH (15 mL) was added sulfuric acid (10 μL, 0.188 mmol) and the reaction mixture was stirred at 70° C. for 18 hrs. The reaction mixture was allowed to cool to room temperature and diluted with 1N aqueous sodium hydroxide solution (5 mL). The mixture was concentrated under reduced pressure and the residue was dissolved in EtOAc. The solution was washed with water, brine, dried over sodium sulfate, filtered off and concentrated under reduced pressure. The residue was purified by column chromatography [silica gel] providing 4-tert-butyl 2-methyl morpholine-2,4-dicarboxylate (300 mg). LCMS (m/z): 246.1 [M+H]+; Rt=0.72 min.

### Step 2: Preparation of 2-methyl-morpholine-2,4-dicarboxylic acid 4-tert-butyl ester 2-methylester

To a solution of diisopropylamine (0.174 mL, 1.223 mmol) in tetrahydrofuran (5 mL) was added n-BuLi (0.764 mL, 1.223 mmol) at 0° C. and the mixture was stirred 0° C. for 1 hr. The mixture was cooled to −78° C. and a solution of 4-tert-butyl 2-methyl morpholine-2,4-dicarboxylate (300 mg, 1.223 mmol) in tetrahydrofuran (5 mL) was added. The reaction mixture was stirred at −78° C. for 1 hr and allowed to warm up slowly to room temperature. The mixture was diluted with saturated aqueous ammonium chloride solution (5 mL) and extracted with EtOAc (3×50 mL). The combined organic layers were washed with water and brine, dried over sodium sulfate, filtered off and concentrated under reduced pressure. The residue was purified by column chromatography [silica gel, EtOAc/heptane=0/100 to 40/60] providing 2-methyl-morpholine-2,4-dicarboxylic acid 4-tert-butyl ester 2-methylester (211 mg). LCMS (m/z): 260.0 [M+H]+; Rt=0.77 min.

### Step 3: Preparation of 4-(tert-butoxycarbonyl)-2-methylmorpholine-2-carboxylic acid

A mixture of 2-methyl-morpholine-2,4-dicarboxylic acid 4-tert-butyl ester 2-methylester (290 mg, 1.118 mmol) and 1N aqueous sodium hydroxide solution (12 mL, 12.00 mmol) in tetrahydrofuran (10 mL) was stirred at 70° C. for 2 hrs. The reaction mixture was cooled to room temperature and concentrated under reduced pressure to remove tetrahydrofuran. The aqueous solution was acidified with 1N aqueous hydrochloride solution until pH˜5 and extracted with EtOAc (3×15 mL). The organic layers were combined and washed with brine before dried over sodium sulfate, filtered off and concentrated under reduced pressure. The residue was purified by column chromatography [silica gel, EtOAc/heptane=0/100 to 70/30] providing 4-(tert-butoxycarbonyl)-2-methylmorpholine-2-carboxylic acid (155 mg). LCMS (m/z): 268.0 [M+Na]+; Rt=0.61 min.

### Synthesis of (3R,5S)-/(3S,5R)-1-(benzyloxycarbonyl)-5-methylpiperidine-3-carboxylic acid [mixture of cis isomers] and (3R,5R)-/(3S,5S)-1-(benzyloxycarbonyl)-5-methylpiperidine-3-carboxylic acid [mixture of trans isomers]

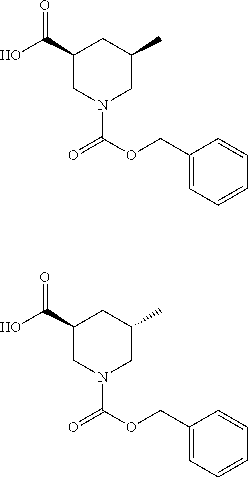

### Step 1: Preparation of methyl 5-methylpiperidine-3-carboxylate (mixture of cis and trans isomers)

A mixture of methyl 5-methylnicotinate (1.06 g, 7.01 mmol), Pd/C (10 wt. %, 100 mg) and platinum(IV)oxide (150 mg, 0.661 mmol) in acetic acid (30 mL) was stirred in a steel bomb under hydrogen atmosphere (200 psi) at 25° C. for 16 hrs. The reaction mixture was filtered through a pad of celite and washed with MeOH (150 mL). The filtrate was concentrated under reduced pressure providing crude methyl 5-methylpiperidine-3-carboxylate (1.5 g; mixture of cis and trans isomers) as a colorless oil, which was directly used in the next step without further purification. LCMS (m/z): 158.1 [M+H]+; Rt=0.32 min.

### Step 2: Preparation of (3R,5S)-/(3S,5R)-5-methyl-piperidine-1,3-dicarboxylic acid 1-benzyl ester 3-methyl ester [cis isomers] and (3R,5R)-/(3S,5S)-5-methyl-piperidine-1,3-dicarboxylic acid 1-benzyl ester 3-methyl ester [trans isomers]

To a mixture of crude methyl 5-methylpiperidine-3-carboxylate (1.5 g, 7.01 mmol) and aqueous sodium carbonate solution (10 wt. %; 20 mL) in tetrahydrofuran (40 mL) was slowly added benzylchloroformate (1.491 mL, 10.45 mmol). The reaction mixture was stirred at 25° C. for 16 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 60/40] providing a mixture of the cis isomers (3R,5S)-/(3S,5R)-5-methyl-piperidine-1,3-dicarboxylic acid 1-benzyl ester 3-methyl ester (1.66 g) as colorless oil and a mixture of the trans isomers (3R,5R)-/(3S,5S)-5-methyl-piperidine-1,3-dicarboxylic acid 1-benzyl ester 3-methyl ester (1.52 g) as colorless oil.

Cis isomers: LCMS (m/z): 292.1 [M+H]+; Rt=0.99 min. Analytical HPLC: Rt=4.04 min.

¹H NMR (300 MHz, chloroform-d) δ [ppm]: 0.92 (d, J=6.45 Hz, 3H) 1.21 (q, J=12.41 Hz, 1H) 1.60 (br. s., 1H) 2.11 (d, J=13.19 Hz, 1H) 2.29 (br. s., 1H) 2.43-2.57 (m, 1 H) 2.75 (br. s., 1H) 3.69 (s, 3H) 4.14 (br. s., 1H) 4.42 (br. s., 1H) 5.14 (br. s., 2H) 7.36 (s, 5H).

Trans isomers: LCMS (m/z): 292.1 [M+H]+; Rt=0.96 min. Analytical HPLC: Rt=3.85 min.

¹H NMR (300 MHz, chloroform-d) δ [ppm]: 0.92 (d, J=6.74 Hz, 3H) 1.47 (br. s., 1H) 1.88-2.07 (m, 2H) 2.67 (br. s., 1H) 2.80-3.09 (m, 1H) 3.30-4.08 (m, 6H) 5.13 (q, J=12.31 Hz, 2H) 7.29-7.39 (m, 5H).

### Step 3-a: Preparation of (3R,5S)-/(3S,5R)-1-(benzyloxycarbonyl)-5-methylpiperidine-3-carboxylic acid [cis isomers]

To the mixture of (3R,5S)-/(3S,5R)-5-methyl-piperidine-1,3-dicarboxylic acid 1-benzyl ester 3-methyl ester (1.66 g, 5.70 mmol) in MeOH (4.5 mL) and water (3 mL) was added 6N aqueous sodium hydroxide solution (1.5 mL, 9.0 mmol). The reaction mixture was stirred at 25° C. for 2 hrs and concentrated under reduced pressure to a volume of −2 mL. The mixture was acidified with 1N aqueous hydrochloride solution until pH˜4, diluted with EtOAc and stirred for 10 min. The separated organic layer was washed with brine, dried sodium sulfate, filtered off and concentrated under reduced pressure providing a mixture of the cis isomers (3R,5S)- and (3S,5R)-1-(benzyloxycarbonyl)-5-methylpiperidine-3-carboxylic acid (1.36 g) as a colorless oil, which was directly used in the next step without further purification. LCMS (m/z): 278.1 [M+H]+; Rt=0.81 min.

### Step 3-b: Preparation of (3R,5R)-/(3S,5S)-1-(benzyloxycarbonyl)-5-methylpiperidine-3-carboxylic acid [trans isomers]

To the mixture of (3R,5S)-/(3S,5R)-5-methyl-piperidine-1,3-dicarboxylic acid 1-benzyl ester 3-methyl ester (1.55 g, 5.32 mmol) in MeOH (4.5 mL) and water (3 mL) was added 6N aqueous sodium hydroxide solution (1.5 mL, 9.0 mmol). The reaction mixture was stirred at 25° C. for 2 hrs and concentrated under reduced pressure to a volume of ˜2 mL. The mixture was acidified with 1N aqueous hydrochloride solution until pH˜4, diluted with EtOAc and stirred for 10 min. The separated organic layer was washed with brine solution, dried over sodium sulfate, filtered off and concentrated under reduced pressure providing a mixture of trans isomers (3R,5R)- and (3S,5S)-1-(benzyloxycarbonyl)-5-methylpiperidine-3-carboxylic acid (1.22 g) as a colorless oil, which was directly used in the next step without further purification. LCMS (m/z): 278.1 [M+H]+; Rt=0.79 min.

### Synthesis of (3S,4R)-1-(benzyloxycarbonyl)-4-methoxypyrrolidine-3-carboxylic acid

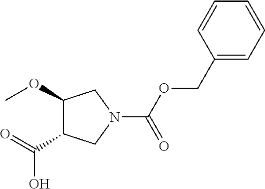

### Step 1: Preparation of (3R,4S)-benzyl-3-methoxy-4-vinylpyrrolidine-1-carboxylate

To a solution of (3R,4S)-benzyl 3-hydroxy-4-vinylpyrrolidine-1-carboxylate (5.3 g, 21.43 mmol) in DMF (25 mL) was added carefully sodium hydride (60 wt. % in mineral oil, 1.714 g, 42.9 mmol) and the mixture was stirred at room temperature for 1 hr. To the mixture was added methyl iodide (4.29 mL, 68.6 mmol) slowly over 30 min and stirring was continued for additional 18 hrs at 25° C. The mixture was diluted with saturated aqueous ammonium chloride solution (10 mL) and with EtOAc (100 mL). The mixture was washed with water and brine, dried over sodium sulfate, filtered off and concentrated under reduced pressure. The residue was purified by column chromatography [silica gel, EtOAc/heptane=0/100 to 50/50] providing (3R,4S)-benzyl-3-methoxy-4-vinylpyrrolidine-1-carboxylate (5.0 g). LCMS (m/z): 262.1 [M+H]+; Rt=0.78 min.

### Step 2: Preparation of (3S,4R)-1-(benzyloxycarbonyl)-4-methoxypyrrolidine-3-carboxylic acid

A mixture of (3R,4S)-benzyl-3-methoxy-4-vinylpyrrolidine-1-carboxylate (5 g, 19.13 mmol), ruthenium trichloride (4.99 g, 19.13 mmol), sodium periodate (16.37 g, 77 mmol) in carbontetrachloride (20 mL), water (20 mL) and acetonitrile (20 mL) was stirred at room temperature overnight. The reaction mixture was diluted with dichloromethane (200 mL) and water (200 mL) and filtered to remove the slur. The separated aqueous layer was washed with dichloromethane (2×200 mL), the combined organic layers were dried over sodium sulfate filtered off and concentrated under reduced pressure. The residue was dissolved in acetone (50 mL) and chromium trioxide (3.05 g, 30.5 mmol) and 1N aqueous sulfuric acid solution (50 mL) were added. The mixture was stirred at room temperature for 3 hrs. The reaction mixture was extracted with dichloromethane (2×100 mL). The combined organic layers were concentrated under reduced pressure and the residue was purified by column chromatography [silica gel] providing (3R,4S)-1-(benzyloxycarbonyl)-4-methoxypyrrolidine-3-carboxylic acid (2.7 g). LCMS (m/z): 280.0 [M+H]+; Rt=0.69 min.

### Synthesis of (3R,5R)-1-(tert-butoxycarbonyl)-5-(methoxymethyl)pyrrolidine-3-carboxylic acid

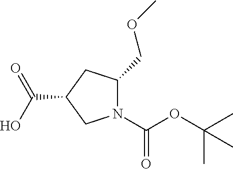

### Step 1: Preparation of (2R,4R)-4-(tert-butyl-diphenyl-silanyloxy)-pyrrolidine-1,2-dicarboxylic acid 1-tert-butyl ester 2-methyl ester

To a solution of (2R,4R)-4-hydroxy-pyrrolidine-1,2-dicarboxylic acid 1-tert-butyl ester 2-methyl ester (5.0 g, 20.22 mmol) in dichloromethane (35 mL) was added imidazole (2.34 g, 34.4 mmol) and tert-butylchlorodiphenylsilane (5.71 mL, 22.24 mmol). The reaction mixture was stirred at room temperature for 18 hrs and filtered through a thin layer of celite. The filtrate was washed with water and brine, dried over sodium sulfate, filtered off and concentrated under reduced pressure providing crude (2R,4R)-4-(tert-butyl-diphenyl-silanyloxy)-pyrrolidine-1,2-dicarboxylic acid 1-tert-butyl ester 2-methyl ester (10.9 g), which was directly used in the next step without further purification. LCMS (m/z): 486.2 [M+H]+; Rt=1.36 min.

### Step 2: Preparation of (2R,4R)-tert-butyl 4-(tert-butyldiphenylsilyloxy)-2-(hydroxymethyl)pyrrolidine-1-carboxylate

To a solution of (2R,4R)-1-tert-butyl 2-methyl 4-(tert-butyldiphenylsilyloxy)pyrrolidine-1,2-dicarboxylate (10.0 g, 20.68 mmol) in tetrahydrofuran (100 mL) was added sodium borohydride (1.564 g, 41.4 mmol) and the mixture was heated at 70° C. for 2 hrs. The reaction mixture was allowed to cool to room temperature and was diluted with saturated aqueous ammonium chloride solution (5 mL) and EtOAc (100 mL). The mixture was washed with water, aqueous sodium bicarbonate solution and brine and concentrated under reduced pressure. The residue was purified by column chromatography [silica gel, 40 g, EtOAc/heptane=0/100 to 70/30] providing (2R,4R)-tert-butyl 4-(tert-butyldiphenylsilyloxy)-2-(hydroxymethyl)pyrrolidine-1-carboxylate (5.0 g). LCMS (m/z): 456.2 [M+H]+; Rt=0.88 min.

### Step 3: Preparation of (2R,4R)-tert-butyl 4-(tert-butyldiphenylsilyloxy)-2-(methoxymethyl)pyrrolidine-1-carboxylate

To a solution of (2R,4R)-tert-butyl 4-(tert-butyldiphenylsilyloxy)-2-(hydroxymethyl)pyrrolidine-1-carboxylate (5.0 g, 10.97 mmol) in tetrahydrofuran (25 mL) was added carefully sodium hydride (0.316 g, 13.17 mmol) and the mixture was stirred at room temperature for 2 hrs. To the mixture was added methyl iodide (1.372 mL, 21.95 mmol) and stirring was continued at 23° C. for 183 hrs. The reaction mixture was diluted carefully with aqueous saturated ammonium chloride solution (10 mL) and EtOAc (100 mL). The mixture was washed with water (2×50 mL) and brine (2×100 mL), dried over sodium sulfate, filtered off and concentrated under reduced pressure. The residue was purified by column chromatography [silica gel, EtOAc/heptane=0/100 to 40/60] providing (2R,4R)-tert-butyl 4-(tert-butyldiphenylsilyloxy)-2-(methoxymethyl)pyrrolidine-1-carboxylate (4.7 g). LCMS (m/z): 470.1 [M+H]+; Rt=1.45 min.

### Step 4: Preparation of (2R,4R)-tert-butyl 4-hydroxy-2-(methoxymethyl)pyrrolidine-1-carboxylate

To a solution of (2R,4R)-tert-butyl 4-(tert-butyldiphenylsilyloxy)-2-(methoxymethyl)pyrrolidine-1-carboxylate (4.60 g, 9.79 mmol) in tetrahydrofuran (30 mL) was added tetrabutylammonium fluoride (2.56 g, 9.79 mmol) and the mixture was stirred at 23° C. for 2 hrs. The reaction mixture was diluted with EtOAc (100 mL) and washed with water, brine, dried over sodium sulfate, filtered off and concentrated under reduced pressure. The residue was purified by column chromatography [silica gel, 400 g, EtOAc/heptane=0/100 to 50/50] providing (2R,4R)-tert-butyl 4-hydroxy-2-(methoxymethyl)pyrrolidine-1-carboxylate (1.0 g). LCMS (m/z): 232.1 [M+H]+; Rt=0.62 min.

### Step 5: Preparation of (2R,4S)-tert-butyl 4-(4-methoxybenzoyloxy)-2-(methoxymethyl)pyrrolidine-1-carboxylate

A mixture of (2R,4R)-tert-butyl 4-hydroxy-2-(methoxymethyl)pyrrolidine-1-carboxylate (1 g, 4.32 mmol), p-anisic acid (0.789 g, 5.19 mmol), N1,N1,N2,N2-tetramethyldiazene-1,2-dicarboxamide (0.744 g, 4.32 mmol), benzene (20 mL) and tributyl phosphine (1.60 mL, 6.49 mmol) in a closed vial was stirred at 60° C. for 2 hrs. The reaction mixture was diluted with EtOAc (100 mL). The mixture was washed with water, brine, dried over sodium sulfate, filtered off and concentrated under reduced pressure. The residue was purified by column chromatography [silica gel] providing (2R,4S)-tert-butyl 4-(4-methoxybenzoyloxy)-2-(methoxymethyl)pyrrolidine-1-carboxylate. (1.2 g). LCMS (m/z): 366.2 [M+H]+; Rt=1.02 min.

### Step 6: Preparation of (2R,4S)-tert-butyl 4-hydroxy-2-(methoxymethyl)pyrrolidine-1-carboxylate

To a solution of (2R,4S)-tert-butyl 4-(4-methoxybenzoyloxy)-2-(methoxymethyl)pyrrolidine-1-carboxylate (1.2 g, 3.28 mmol) in tetrahydrofuran (20 mL) was added 3N aqueous sodium hydroxide solution (20 mL) and the mixture was stirred at 70° C. for 18 hrs. The reaction mixture was allowed to cool to room temperature and diluted with water (50 mL). The mixture was extracted with EtOAc (2×100 mL). The combined organic layers were washed with water (50 mL), brine (2×100 mL), dried over sodium sulfate, filtered off and concentrated under reduced pressure. The residue was purified by column chromatography [silica gel] providing (2R,4S)-tert-butyl 4-hydroxy-2-(methoxymethyl)pyrrolidine-1-carboxylate (600 mg). LCMS (m/z): 232.1 [M+H]+; Rt=0.62 min.

### Step 7: Preparation of (2R,4S)-tert-butyl 2-(methoxymethyl)-4-(methylsulfonyloxy)pyrrolidine-1-carboxylate

A mixture of (2R,4S)-tert-butyl 4-hydroxy-2-(methoxymethyl)pyrrolidine-1-carboxylate (600 mg, 2.59 mmol), N,N-diisopropyl-N-ethylamine (0.544 mL, 3.11 mmol) and methanesulfonyl chloride (357 mg, 3.11 mmol) in dichloromethane (10 mL) was stirred at 23° C. for 18 hrs. The reaction mixture was concentrated under reduced pressure and the residue was purified by column chromatography [silica gel] (2R,4S)-tert-butyl 2-(methoxymethyl)-4-(methylsulfonyloxy)pyrrolidine-1-carboxylate (650 mg). LCMS (m/z): 310.1 [M+H]+; Rt=0.90 min.

### Step 8: Preparation of (2R,4R)-tert-butyl 4-cyano-2-(methoxymethyl)pyrrolidine-1-carboxylate

To a solution of (2R,4S)-tert-butyl 2-(methoxymethyl)-4-(methylsulfonyloxy)pyrrolidine-1-carboxylate (910 mg, 2.94 mmol) in DMF (15 mL) was added tetrabutylammonium cyanide (948 mg, 3.53 mmol) and the mixture was stirred at 60° C. for 18 hrs. The reaction mixture was diluted with EtOAc (50 mL) and washed with water (2×) and brine. The organic layer was dried over sodium sulfate, filtered off and concentrated under reduced pressure. The residue was purified by column chromatography [silica gel, EtOAc/heptane=0/100 to 50/50] providing (2R,4R)-tert-butyl 4-cyano-2-(methoxymethyl)pyrrolidine-1-carboxylate (250 mg). LCMS (m/z): 185.0 [M+H, loss of t-Bu]+; Rt=0.78 min.

### Step 9: Preparation of (3R,5R)-1-(tert-butoxycarbonyl)-5-(methoxymethyl)pyrrolidine-3-carboxylic acid

A mixture of (2R,4R)-tert-butyl 4-cyano-2-(methoxymethyl)pyrrolidine-1-carboxylate (250 mg, 1.040 mmol), 6N aqueous sodium hydroxide solution (5.72 mL, 34.3 mmol) and EtOH (7 mL) in a closed vial was stirred at 85° C. for 30 min. The reaction mixture was allowed to cool to room temperature, acidified with 1N aqueous hydrochloride solution until pH˜5 and extracted with dichloromethane (3×100 mL). The combined organic layers were concentrated under reduced pressure and the residue was dissolved in EtOAc. The organic layer was washed with water, brine, dried over sodium sulfate filtered off and concentrated under reduced pressure providing crude (3R,5R)-1-(tert-butoxycarbonyl)-5-(methoxymethyl)pyrrolidine-3-carboxylic acid (210 mg), which was directly used in the next step without further purification. LCMS (m/z): 282.0 [M+Na]+; Rt=0.68 min. ¹H NMR (400 MHz, methanol-d4) δ [ppm]: 1.46 (s, 9H) 2.08-2.22 (m, 2H) 3.15-3.27 (m, 1H) 3.34 (s, 3H) 3.44 (d, J=4.70 Hz, 2H) 3.46-3.61 (m, 2H) 3.94-4.05 (m, 1H).

### Synthesis of 1-(benzyloxycarbonyl)-5-fluoropiperidine-3-carboxylic acid [cis isomers]

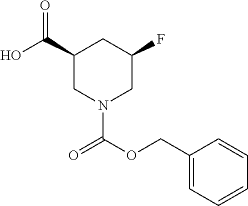

### Step 1: Preparation of 1-benzyl-5-hydroxypiperidine-3-carboxylic acid

To a mixture of 5-hydroxypiperidine-3-carboxylic acid (3 g, 20.67 mmol) and potassium carbonate (4.41 g, 31.9 mmol) in MeOH (48 mL) and water (24 mL) was added slowly a solution of benzyl bromide (2.58 mL, 21.70 mmol) in MeOH (2.00 mL). The mixture was stirred for ˜3 hrs at room temperature. The volatile solvent was removed under reduced pressure and the remaining solution was carefully acidified with 1N aqueous hydrochloride solution (˜100 mL). The aqueous solution was concentrated under reduced pressure to dryness. The residue was suspended in MeOH (˜50 mL) and filtered off. To the filtrate was added sodium methoxide in MeOH (25 wt. %, 6.8 g) and the reaction mixture was stirred for ˜18 hrs. The mixture was filtered and concentrated under reduced pressure providing crude 1-benzyl-5-hydroxypiperidine-3-carboxylic acid as a solid, which was directly used in the next reaction without further purification. LCMS (m/z): 336.0 [M+H]+; Rt=0.36 min.

### Step 2: Preparation of methyl 1-benzyl-5-hydroxypiperidine-3-carboxylate

Chlorotrimethylsilane (17.11 mL, 134 mmol) was added slowly to a solution of crude 1-benzyl-5-hydroxypiperidine-3-carboxylic acid (4.5 g, 19.13 mmol) in MeOH (90 mL). The mixture was stirred for ˜18 hrs and concentrated under reduced pressure. The residue was purified by column chromatography [silica gel, 80 g, 30 min, EtOAc/heptane=20/80 to 70/30] providing methyl 1-benzyl-5-hydroxypiperidine-3-carboxylate (3.37 g, 71% over 2 steps) as a colorless oil. LCMS (m/z): 250.3 [M+H]+; Rt=0.36 min.

### Step 3: Preparation of a mixture of (3S,5R)-/(3R,5S)-methyl 1-benzyl-5-fluoropiperidine-3-carboxylate [cis isomers] and (3R,5R)-/(3S,5S)-methyl 1-benzyl-5-(fluoromethyl)pyrrolidine-3-carboxylate [cis isomers]

To methyl 1-benzyl-5-hydroxypiperidine-3-carboxylate (2 g, 8.02 mmol) in DCM (32 mL) at −78° C. was added dropwise DAST (2.12 mL, 16.04 mmol). The mixture was allowed to warm slowly to room temperature over ˜16 hrs. The reaction mixture was diluted with saturated aqueous sodium bicarbonate solution. The separated aqueous layer was extracted with dichloromethane (2×). The combined organic layers were concentrated under reduced pressure. The residue was purified by column chromatography [silica gel, 40 g, 30 min, EtOAc/heptane=0/100 to 40/60] providing a mixture of methyl 1-benzyl-5-fluoropiperidine-3-carboxylate [cis isomers] and methyl 1-benzyl-5-(fluoromethyl)pyrrolidine-3-carboxylate [cis isomers] (1.80 g) as a slightly orange oil. LCMS (m/z): 252.1 [M+H]+; Rt=0.41 min.

### Step 4: Preparation of mixture of methyl 5-fluoropiperidine-3-carboxylate acetic acid salt [cis isomers] and methyl 5-(fluoromethyl)pyrrolidine-3-carboxylate acetic acid salt [cis isomers]

To the mixture of methyl 1-benzyl-5-fluoropiperidine-3-carboxylate [cis isomers] and methyl 1-benzyl-5-(fluoromethyl)pyrrolidine-3-carboxylate [cis isomers] (1.8 g, 7.16 mmol) in acetic acid (14 mL) was added Pd/C (10 wt. %, 170 mg) and platinum(IV)oxide (240 mg, 1.057 mmol). The mixture was hydrogenated in a steel bomb for ˜16 hrs (pressure: 1400 psi). The catalyst was filtered off through celite and the clear solution was concentrated under reduced pressure providing crude mixture of methyl 5-fluoropiperidine-3-carboxylate acetic acid salt [cis isomers] and methyl 5-(fluoromethyl)pyrrolidine-3-carboxylate acetic acid salt [cis isomers] as a slightly yellowish oil, which was directly used in the next reaction without further purification. LCMS (m/z): 162.0 [M+H]+; Rt=0.19 min.

### Step 5: Preparation of (3R,5S)-/(3S,5R)-5-fluoro-piperidine-1,3-dicarboxylic acid 1-benzyl ester 3-methyl ester [cis isomers] and (3R,5R)/(3S,5S)-5-fluoromethyl-pyrrolidine-1,3-dicarboxylic acid 1-benzyl ester 3-methyl ester [cis isomers]

To a mixture of crude methyl 5-fluoropiperidine-3-carboxylate (1.584 g, 7.16 mmol) acetic acid salt in tetrahydrofuran (15 mL) was added aqueous sodium carbonate solution (10 wt. %, ˜7 mL) until pH˜8-9. Benzyl chloroformate (1.145 mL, 8.02 mmol) was added slowly and saturated aqueous sodium bicarbonate solution was added. The reaction mixture was stirred for 1 hr and was diluted with EtOAc. The separated organic phase was washed with saturated aqueous sodium bicarbonate solution (2×) and concentrated under reduced pressure. The residue was dissolved in EtOAc, dried over sodium sulfate, filtered off and concentrated under reduced pressure. The residue was purified by column chromatography [silica gel, 40 g, 16 min, EtOAc/heptane=0/100 to 40/60]. Fractions were combined and concentrated under reduced pressure providing Fraction 1: 1.005 g (ratio of isomers: 90:10); Fractions 2: 459 mg (ratio of isomers: 50:50). Fractions 2 was dissolved in DMSO and purified by HPLC [˜50 mg/l mL of DMSO]. Fractions of P1 and P2 were collected and lyophilized providing cis isomers and trans isomers of 1-benzyl 3-methyl 5-fluoropiperidine-1,3-dicarboxylate as colorless oils.

Fraction 1/Fraction P1: 5-Fluoro-piperidine-1,3-dicarboxylic acid 1-benzyl ester 3-methyl ester [cis isomers]

Yield: 143 mg; LCMS (m/z): 296.0 [M+H]+; Rt=0.83 min. ¹H NMR (400 MHz, DMSO-d6, 70° C.) δ [ppm]: 7.21-7.48 (m, 5H), 5.07-5.15 (m, 2H), 4.54-4.76 (m, 1H), 3.75-3.95 (m, 2H), 3.58-3.63 (m, 3H), 3.26-3.38 (m, 1H), 3.17-3.27 (m, 1H), 2.68 (ttd, J=9.2, 4.5, 1.6 Hz, 1H), 2.27 (ddt, J=17.6, 13.2, 4.2 Hz, 1H), 1.89 (br. s., 1 H)

Fraction P2: 5-Fluoromethyl-pyrrolidine-1,3-dicarboxylic acid 1-benzyl ester 3-methyl ester [cis isomers]

Yield: 118 mg; LCMS (m/z): 296.0 [M+H]+; Rt=0.85 min. ¹H NMR (400 MHz, DMSO-d6, 70° C.) δ [ppm]: 7.14-7.58 (m, 5H), 5.09 (d, J=5.0 Hz, 2H), 4.46-4.64 (m, 1H), 4.40 (d, J=3.4 Hz, 1H), 3.96-4.15 (m, 1H), 3.80 (dd, J=10.6, 8.2 Hz, 1H), 3.35-3.49 (m, 1H), 3.16 (quin, J=8.0 Hz, 1H), 3.09 (s, 3H), 2.26-2.45 (m, 1H), 2.04-2.13 (m, 1H)

### Step 6: Preparation of (3R,5S)-/(3S,5R)-1-(benzyloxycarbonyl)-5-fluoropiperidine-3-carboxylic acid [cis isomers]

To a solution of Fraction 1 (5-fluoro-piperidine-1,3-dicarboxylic acid 1-benzyl ester 3-methyl ester [cis isomers]; 500 mg, 1.693 mmol) in MeOH (10 mL) was added slowly 2N aqueous sodium hydroxide solution (10 mL). The mixture was stirred for ˜10 min at room temperature. The mixture was acidified with 1N aqueous hydrochloride solution and the volatile solvent was removed under reduced pressure. The residue was diluted with EtOAc. The separated organic layer was washed with brine, dried over sodium sulfate, filtered off and concentrated under reduced pressure providing crude mixture of (3R,5S)-/(3S,5R)-1-(benzyloxycarbonyl)-5-fluoropiperidine-3-carboxylic acid [cis isomers] (487 mg) as a white solid, which was directly used in the next reaction without further purification. LCMS (m/z): 282.0 [M+H]+; Rt=0.70 min.

### Synthesis of (3S,5S)-/(3R,5R)-1-(benzyloxycarbonyl)-5-(fluoromethyl)pyrrolidine-3-carboxylic acid [cis isomers]

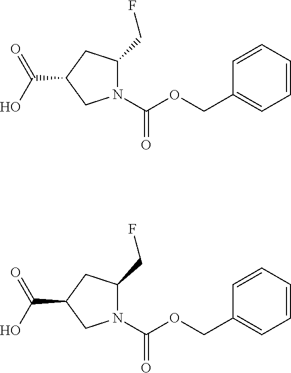

To a solution of Fraction P2 (5-fluoromethyl-pyrrolidine-1,3-dicarboxylic acid 1-benzyl ester 3-methyl ester [cis isomers]; 70 mg, 0.237 mmol) in MeOH (8 mL) was added slowly 2N aqueous sodium hydroxide solution (8 mL). The mixture was stirred for ˜5 min at room temperature. The mixture was partially concentrated under reduced pressure and was acidified with 1N aqueous hydrochloride solution and diluted with EtOAc. The separated aqueous layer was extracted with EtOAc (2×). The combined organic layers were dried over sodium sulfate, filtered off and concentrated under reduced pressure providing crude mixture of (3S,5S)-/(3R,5R)-1-(benzyloxycarbonyl)-5-(fluoromethyl)pyrrolidine-3-carboxylic acid [cis isomers] (56 mg) as a colorless oil, which was directly used in the next reaction without further purification. LCMS (m/z): 282.1 [M+H]+; Rt=0.71 min.

### Synthesis of (3R,5S)-/(3S,5R)-1-(benzyloxycarbonyl)-5-(trifluoromethyl)piperidine-3-carboxylic acid and (3R,5R)-/(3S,5S)-1-(benzyloxycarbonyl)-5-(trifluoromethyl)piperidine-3-carboxylic acid

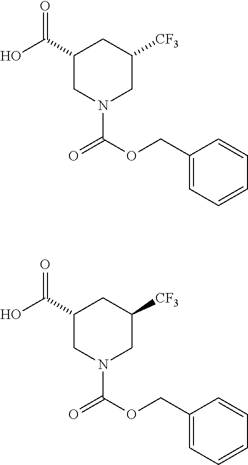

### Step 1: Preparation of methyl 5-(trifluoromethyl)nicotinate

To a solution of 5-(trifluoromethyl)nicotinic acid (1.0 g, 5.08 mmol) in MeOH (10 mL) was added slowly thionyl chloride (0.926 mL, 12.69 mmol). The reaction mixture was stirred at 45° C. for 18 hrs and then concentrated under reduced pressure. The residue was dissolved in dichloromethane and the organic layer was washed with saturated aqueous sodium bicarbonate solution, water and brine, dried over sodium sulfate, filtered off and concentrated under reduced pressure providing crude methyl 5-(trifluoromethyl)nicotinate (736 mg) as oil, which was directly used in the next step without further purification. LCMS (m/z): 206.0 [M+H]+; Rt=0.72 min.

### Step 2: Preparation of methyl 5-(trifluoromethyl)piperidine-3-carboxylate (mixture of cis and trans isomers)

A mixture of methyl 5-(trifluoromethyl)nicotinate (736 mg, 3.59 mmol), Pd/C (10 wt. %, 36 mg) and platinum(IV)oxide (52.5 mg, 0.231 mmol) in acetic acid (11 mL) was stirred in a steel bomb under hydrogen atmosphere (200 psi) at 25° C. for 20 hrs. The reaction mixture was filtered through a pad of celites and washed with MeOH (50 mL). The filtrate was concentrated under reduced pressure providing crude methyl 5-(trifluoromethyl)piperidine-3-carboxylate (936 mg; mixture of cis and trans isomers) as a colorless oil, which was directly used in the next step without further purification. LCMS (m/z): 212.0 [M+H]+; Rt=0.38 min.

### Step 3: Preparation of (3R,5S)-/(3S,5R)-5-trifluoromethyl-piperidine-1,3-dicarboxylic acid 1-benzyl ester 3-methyl ester [cis isomers] and (3R,5R)-/(3S,5S)-5-trifluoromethyl-piperidine-1,3-dicarboxylic acid 1-benzyl ester 3-methyl ester [trans isomers]

To a mixture of crude methyl 5-(trifluoromethyl)piperidine-3-carboxylate (953 mg, 3.61 mmol) aqueous sodium carbonate solution (10 wt. %; 5.13 mL) in tetrahydrofuran (15 mL) was added slowly benzylchloroformate (0.58 mL, 4.04 mmol). The reaction mixture was stirred at 25° C. for 2 hrs. The mixture was diluted with EtOAc and stirred for additional 30 min. The separated organic layer was washed with saturated aqueous sodium bicarbonate solution, water and brine solution. The organic phase was dried over sodium sulfate, filtered off and concentrated under reduced pressure. The residue was purified by column chromatography [silica gel, 24 g, EtOAc/heptane=0/100 to 30/70] providing a mixture of the cis isomers (3R,5S)-/(3S,5R)-5-trifluoromethyl-piperidine-1,3-dicarboxylic acid 1-benzyl ester 3-methyl ester (296 mg) as a white solid and a mixture of the trans isomers (3R,5R)-/(3S,5S)-5-trifluoromethyl-piperidine-1,3-dicarboxylic acid 1-benzyl ester 3-methyl ester (240 mg) as an oil.

Cis isomers: LCMS (m/z): 346.0 [M+H]+; Rt=1.01 min. Analytical HPLC: Rt=4.22 min.

Trans isomers: LCMS (m/z): 346.1 [M+H]+; Rt=0.98 min. Analytical HPLC: Rt=4.09 min.

### Step 4-a: Preparation of (3R,5S)-/(3S,5R)-1-(benzyloxycarbonyl)-5-(trifluoromethyl)piperidine-3-carboxylic acid [cis isomers]

To a mixture of the cis isomers (3R,5S)-/(3S,5R)-1-benzyl 3-methyl 5-(trifluoromethyl)piperidine-1,3-dicarboxylate (296 mg, 0.857 mmol) in MeOH (0.9 mL) and water (0.6 mL) was added 6N aqueous sodium hydroxide solution (0.3 mL, 1.8 mmol). The reaction mixture was stirred at 25° C. for 1 hr and concentrated under reduced pressure to a volume of ˜0.5 mL. The mixture was acidified with 1N hydrochloride solution until pH˜4, diluted with EtOAc and stirred for 10 min. The separated organic layer was washed with brine solution, dried over sodium sulfate, filtered off and concentrated under reduced pressure providing a mixture of (3R,5S)- and (3S,5R)-1-(benzyloxycarbonyl)-5-(trifluoromethyl)piperidine-3-carboxylic acid (254 mg) as a colorless oil, which was directly used in the next step without further purification. LCMS (m/z): 332.0 [M+H]+; Rt=0.91 min.

### Step 4-b: Preparation of (3R,5R)-/(3S,5S)-1-(benzyloxycarbonyl)-5-(trifluoromethyl)piperidine-3-carboxylic acid [trans isomers]

To a mixture of the trans isomers (3R,5R)-/(3S,5S)-1-benzyl 3-methyl 5-(trifluoromethyl)piperidine-1,3-dicarboxylate (1.55 g, 5.32 mmol) in MeOH (0.75 mL) and water (0.5 mL) was added 6N aqueous sodium hydroxide solution (0.25 mL, 1.5 mmol). The reaction mixture was stirred at 25° C. for 2 hrs and concentrated under reduced pressure to a volume of ˜0.5 mL. The mixture was acidified with 1N hydrochloride until pH˜4, diluted with EtOAc and stirred for 10 min. The separated organic layer was washed with brine, dried over sodium sulfate, filtered off and concentrated under reduced pressure providing a mixture of (3R,5R)-/(3S,5S)-1-(benzyloxycarbonyl)-5-(trifluoromethyl)piperidine-3-carboxylic acid (218 mg) as a colorless oil, which was directly used in the next step without further purification. LCMS (m/z): 332.1 [M+H]+; Rt=0.83 min

### Synthesis of (3R,6S)-/(3S,6R)-1-(benzyloxycarbonyl)-6-methylpiperidine-3-carboxylic acid and (3R,6R)-/(3S,6S)-1-(benzyloxycarbonyl)-6-methylpiperidine-3-carboxylic acid

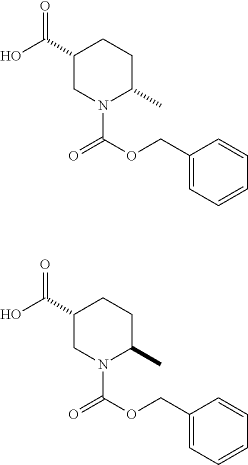

### Step 1: Preparation of methyl 6-methylpiperidine-3-carboxylate (mixture of cis and trans isomers)

A mixture of methyl 6-methylnicotinate (1.52 g, 10 mmol), Pd/C (10 wt. %, 100 mg) and platinum(IV)oxide (150 mg, 0.661 mmol) in acetic acid (16 mL) was stirred in a steel bomb under hydrogen atmosphere (200 psi) at 25° C. for 16 hrs. The reaction mixture was filtered through a pad of celites and washed with MeOH (150 mL). The filtrate was concentrated under reduced pressure providing crude methyl 6-methylpiperidine-3-carboxylate (2.5 g; mixture of cis and trans isomers) as a colorless oil, which was directly used in the next step without further purification. LCMS (m/z): 158.1 [M+H]+; Rt=0.28 min.

### Step 2: Preparation of (3R,6S)-/(3S,6R)-6-methyl-piperidine-1,3-dicarboxylic acid 1-benzyl ester 3-methyl ester [cis isomers] and (3R,6R)-/(3S,6S)-6-methyl-piperidine-1,3-dicarboxylic acid 1-benzyl ester 3-methyl ester [trans isomers]

To a mixture of crude methyl 6-methylpiperidine-3-carboxylate (2.33 g, 10 mmol) aqueous sodium carbonate solution (10 wt. %; 20 mL) in tetrahydrofuran (40 mL) was added slowly benzylchloroformate (1.431 mL, 10.03 mmol). The reaction mixture was stirred at 25° C. for 2 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 40/60] providing a mixture of the cis isomers (3R,6S)-/(3S,6R)-6-methyl-piperidine-1,3-dicarboxylic acid 1-benzyl ester 3-methyl ester (1.74 g) as colorless oil and a mixture of the trans isomers (3R,6R)-/(3S,6S)-6-methyl-piperidine-1,3-dicarboxylic acid 1-benzyl ester 3-methyl ester (0.725 g) as a solid.

Cis isomers: LCMS (m/z): 292.1 [M+H]+; Rt=0.95 min. Analytical HPLC: Rt=3.91 min.

¹H NMR (400 MHz, methanol-d4) δ [ppm]: 1.16 (d, J=7.04 Hz, 3H) 1.58-1.83 (m, 3 H) 1.86-1.95 (m, 1H) 2.43 (tt, J=11.74, 4.30 Hz, 1H) 2.98 (t, J=12.91 Hz, 1H) 3.68 (s, 3H) 4.15-4.25 (m, 1H) 4.39-4.49 (m, 1H) 5.12 (s, 2H) 7.27-7.38 (m, 5H).

Trans isomers: LCMS (m/z): 292.1 [M+H]+; Rt=0.93 min. Analytical HPLC: Rt=3.75 min.

¹H NMR (400 MHz, methanol-d4) δ [ppm]: 1.11-1.23 (m, 3H) 1.38-1.47 (m, 1H) 1.76-2.06 (m, 3H) 2.66 (br. s., 1H) 3.19 (dd, J=13.89, 4.11 Hz, 1H) 3.58 (s, 3H) 4.33-4.46 (m, 2H) 5.02-5.08 (m, 1H) 5.10-5.19 (m, 1H) 7.27-7.39 (m, 5H)

### Step 3-a: Preparation of (3R,6S)-/(3S,6R)-1-(benzyloxycarbonyl)-6-methylpiperidine-3-carboxylic acid [cis isomers]

To a mixture of the cis isomers (3R,6S)-/(3S,6R)-6-methyl-piperidine-1,3-dicarboxylic acid 1-benzyl ester 3-methyl ester (1.55 g, 4.84 mmol) in MeOH (4.5 mL) and water (3 mL) was added 6N aqueous sodium hydroxide solution (1.5 mL, 9 mmol). The reaction mixture was stirred at 25° C. for 2 hrs and concentrated under reduced pressure to a volume of ˜2 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of (3R,6S)- and (3S,6R)-1-(benzyloxycarbonyl)-6-methylpiperidine-3-carboxylic acid (1.56 g) as a colorless oil, which was directly used in the next step without further purification. LCMS (m/z): 278.1 [M+H]+; Rt=0.79 min.

### Step 3-b: Preparation of (3R,6R)-/(3S,6S)-1-(benzyloxycarbonyl)-6-methylpiperidine-3-carboxylic acid [trans isomers]

To a mixture of the trans isomers (3R,6R)-/(3S,6S)-6-methyl-piperidine-1,3-dicarboxylic acid 1-benzyl ester 3-methyl ester (884 mg, 3.03 mmol) in MeOH (3 mL) and water (2 mL) was added 6N aqueous sodium hydroxide solution (1.0 mL, 6.0 mmol). The reaction mixture was stirred at 25° C. for 2 hrs and concentrated under reduced pressure to a volume of ˜2 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of (3R,6R)-/(3S,6S)-1-(benzyloxycarbonyl)-6-methylpiperidine-3-carboxylic acid (870 mg) as a white solid, which was directly used in the next step without further purification. LCMS (m/z): 278.1 [M+H]+; Rt=0.77 min

### Synthesis of 4-(tert-butoxycarbonyl)-1,4-oxazepane-6-carboxylic acid

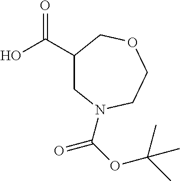

### Step 1: Preparation of tert-butyl 6-methylene-1,4-oxazepane-4-carboxylate

To sodium hydride (60 wt. % in mineral oil, 2.464 g, 61.6 mmol) in DMF (50 mL) was added 3-chloro-2-(chloromethyl)prop-1-ene (3.5 g, 28.0 mmol) at ˜5° C. (ice bath) and a solution of tert-butyl(2-hydroxyethyl)carbamate (4.51 g, 28.0 mmol) in tetrahydrofuran (50 mL). The reaction mixture was stirred at 20-30° C. for ˜2 hrs and concentrated under reduced pressure to remove tetrahydrofuran. The resulting mixture was poured into water and extracted with EtOAc. The combined organic extracts were washed with brine, dried over sodium sulfate, filtered off and concentrated under reduced pressure. The residue was purified by column chromatography [silica gel, 80 g, EtOAc/heptane=0/100 to 50/50] providing tert-butyl 6-methylene-1,4-oxazepane-4-carboxylate (4 g) as a colorless oil. ¹H NMR (400 MHz, chloroform-d) δ [ppm]: 1.46 (s, 9H) 3.33-3.62 (m, 2H) 3.62-3.82 (m, 2H) 4.09 (m, 2H) 4.16 (m, 2H) 4.99 (m, 2H).

### Step 2: Preparation of tert-butyl 6-(hydroxymethyl)-1,4-oxazepane-4-carboxylate

To a solution of tert-butyl 6-methylene-1,4-oxazepane-4-carboxylate (3.2 g, 15.0 mmol) in tetrahydrofuran (15 mL) was added borane tetrahydrofuran (1M solution in tetrahydrofuran, 13.50 mL) at 25° C. via a syringe. The colorless mixture was stirred at room temperature for 3 hrs. The reaction mixture was cooled to 0° C. and 3N aqueous sodium hydroxide solution (5 mL, 15.00 mmol) and aqueous hydrogen peroxide (˜30 wt. %, 2 mL, 19.6 mmol) were added sequentially. The obtained white cloudy mixture was stirred overnight and diluted with pentane. The separated organic layer was dried over potassium carbonate, filtered off and concentrated under reduced pressure. The residue was purified by column chromatography [silica gel, 40 g, EtOAc/heptane=0/100 to 50/50] providing tert-butyl 6-(hydroxymethyl)-1,4-oxazepane-4-carboxylate (2.6 g) as a colorless oil.

### Step 3: Preparation of tert-butyl 6-formyl-1,4-oxazepane-4-carboxylate

To a solution of tert-butyl 6-(hydroxymethyl)-1,4-oxazepane-4-carboxylate (0.9 g, 3.89 mmol) in (15 mL) was added Dess-Martin periodinane (1.650 g, 3.89 mmol) and the mixture was stirred at room temperature for ˜64 hrs. The reaction mixture was diluted with dichloromethane (60 mL) and washed with water, saturated aqueous sodium bicarbonate solution and brine. The organic layer was dried over sodium sulfate, filtered off and concentrated under reduced pressure providing crude tert-butyl 6-formyl-1,4-oxazepane-4-carboxylate (0.45 g) of nearly colorless oil, which was directly used in the next reaction.

### Step 4: Preparation of 4-(tert-butoxycarbonyl)-1,4-oxazepane-6-carboxylic acid

To a mixture of tert-butyl 6-formyl-1,4-oxazepane-4-carboxylate (0.45 g, 1.963 mmol) in tert-butanol (5 mL) was added sodium chlorite (0.231 g, 2.55 mmol) and sodium dihydrogen phosphate (0.306 g, 2.55 mmol) in water (1 mL) at 0° C. The mixture was allowed to warm to room temperature and stirred for about 16 hrs. The mixture was filtered and the filtrate was poured into water and extracted with EtOAc. The combined organic extracts were washed with brine, dried with sodium sulfate, filtered off and concentrated under reduced pressure providing 4-(tert-butoxycarbonyl)-1,4-oxazepane-6-carboxylic acid (0.73 g) as a colorless oil, which was directly used in the next step without further purification. LCMS (m/z): 190.1 [M+H, loss of t-Bu]+; Rt=0.60 min. ¹H NMR (400 MHz, chloroform-d) δ [ppm]: 1.38-1.57 (br. s, 9H) 2.92-3.24 (m, 1H) 3.28-3.44 (m, 1H) 3.47-4.19 (m, 7H).

### Synthesis of 1-(tert-butoxycarbonyl)azepane-3-carboxylic acid

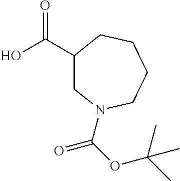

### Step 1: Preparation of ethyl 3-(allylamino)propanoate

To a solution of allyl amine (2.62 mL, 35.0 mmol) in EtOH (50 mL) was added ethyl acrylate (3.81 mL, 35.0 mmol) at 25° C. and the mixture was stirred under argon for ˜16 hrs. The mixture was concentrated under reduced pressure providing crude ethyl 3-(allylamino)propanoate (5.5 g) as an oil, which was used in the next step without further purification.

### Step 2: Preparation of ethyl 3-(allyl(tert-butoxycarbonyl)amino)propanoate

To a solution of ethyl 3-(allylamino)propanoate (5.50 g, 35.0 mmol) in dichloromethane (50 mL) was added sequentially diisopropylamine (6.11 mL, 35.0 mmol), DMAP (0.428 g, 3.50 mmol) and di-tert-butyl dicarbonate (8.13 mL, 35 mmol). The mixture was stirred at room temperature under argon for about 16 hrs. The reaction mixture was poured into water and extracted with dichloromethane. The organic extracts were combined, washed with brine, dried over sodium sulfate, filtered off and concentrated under reduced pressure providing ethyl 3-(allyl(tert-butoxycarbonyl)amino)propanoate (9.12 g) as a yellow oil, which was used in the next step without further purification. LCMS (m/z): 258.1 [M+H], 158.1 [M+H, loss of Boc group]+; Rt=0.95 min.

### Step 3: Preparation of ethyl 2-((allyl(tert-butoxycarbonyl)amino)methyl)pent-4-enoate

To a solution of ethyl 3-(allyl(tert-butoxycarbonyl)amino)propanoate (2 g, 7.77 mmol) in tetrahydrofuran (20 mL) was added lithium bis(trimethylsilyl)amide (8.55 mL, 8.55 mmol) slowly at −78° C. The mixture was stirred for 1 hr and allyl iodide (0.787 mL, 8.55 mmol) was added. The reaction mixture was allowed to warm slowly to room temperature and stirring was continued for 16 hrs. The reaction mixture was poured into water and extracted with EtOAc. The organic extracts were combined, washed with brine, dried with sodium sulfate, filtered off and concentrated under reduced pressure providing ethyl 2-((allyl(tert-butoxycarbonyl)amino)methyl)pent-4-enoate (2.15 g) as a brown oil, which was directly used in the next step without further purification. LCMS (m/z): 198.1 [M+H, loss of Boc group]+; Rt=1.11 min.

### Step 4: Preparation of 2,3,4,7-tetrahydro-azepine-1,3-dicarboxylic acid 1-tert-butyl ester 3-ethyl ester

To a solution of crude ethyl 2-((allyl(tert-butoxycarbonyl)amino)methyl)pent-4-enoate (2.15 g, 7.23 mmol) in dichloromethane (400 mL) under argon was added bis(tricyclohexylphosphine)benzylidine ruthenium(IV) chloride (Grubbs I catalyst; 0.605 g, 0.723 mmol). The reaction mixture was heated to reflux (45 to 65° C. oil bath temperature) for ˜5 hrs. The solvent was removed under reduced pressure and the residue was purified by column chromatography [silica gel, 80 g, EtOAc/heptane=0/100 to 30/70] providing 2,3,4,7-tetrahydro-azepine-1,3-dicarboxylic acid 1-tert-butyl ester 3-ethyl ester (1.84 g) as a black oil. LCMS (m/z): M+1=170.1 [M+H, loss of Boc group]+; Rt=0.96 min.

### Step 5: Preparation of azepane-1,3-dicarboxylic acid 1-tert-butyl ester 3-ethyl ester

To a solution of 2,3,4,7-tetrahydro-azepine-1,3-dicarboxylic acid 1-tert-butyl ester 3-ethyl ester (1.6 g, 5.94 mmol) in MeOH (40 mL) and tetrahydrofuran (10 mL) was added Pd/C (10 wt. %, 0.632 g). The mixture was stirred under hydrogen (balloon) for about 60 hrs. The reaction mixture was diluted with dichloromethane and filtered through celite pad. The filtrate was concentrated under reduced pressure and the residue was purified by column chromatography [silica gel, 80 g, EtOAc/heptane=0/100 to 20/80] providing azepane-1,3-dicarboxylic acid 1-tert-butyl ester 3-ethyl ester (0.6 g) as a brown oil.

### Step 6: Preparation of 1-(tert-butoxycarbonyl)azepane-3-carboxylic acid

To a solution of azepane-1,3-dicarboxylic acid 1-tert-butyl ester 3-ethyl ester (0.6 g, 2.211 mmol) in tetrahydrofuran (8 mL) was added 1N aqueous lithium hydroxide solution (2.65 mL, 2.65 mmol). The mixture was stirred at room temperature for 16 hrs and then was heated to 55° C. for 16 hrs. The reaction mixture was diluted with dichloromethane (10 mL) and extracted with 1N aqueous sodium hydroxide solution (2×20 mL). The aqueous extracts were acidified with 10% aqueous hydrochloride solution until pH˜5 and extracted with EtOAc. The organic extracts were washed with brine, dried with sodium sulfate, filtered off and concentrated under reduced pressure providing crude 1-(tert-butoxycarbonyl)azepane-3-carboxylic acid (0.4 g) as a colorless oil. ¹H NMR (400 MHz, chloroform-d) δ [ppm]: 1.36-1.52 (br. s, 9H) 1.52-2.10 (m, 6H) 2.65-2.98 (m, 1H) 3.04-3.72 (m, 3H) 3.72-3.97 (m, 1H).

### Synthesis of 1-benzyl-6,6-dimethylpiperidine-3-carboxylic acid

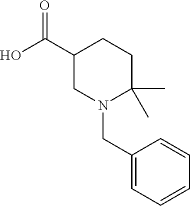

### Step 1: Preparation of 1-phenyl-N-(propan-2-ylidene)methanamine

To a well mixed mixture of acetone (4.65 g, 80 mmol) and basic alumina (15 g) was added a pre-mixed mixture of benzylamine (8.57 g, 80 mmol) and basic alumina (20 g) in portions under gentle shaking. The resultant mixture was hand shaked for 5 min and let stand for ˜1.5 days. The mixture was extracted with dichloromethane (3×15 mL). The combined organic layers were concentrated under reduced pressure and were further dried in high vacuo for 1 day at 60° C. providing crude 1-phenyl-N-(propan-2-ylidene)methanamine (6.3 g) as a light yellow oil, which was directly used in the next step. ¹H NMR (300 MHz, chloroform-d) δ [ppm]: 1.93 (s, 3H) 2.09 (s, 3H) 4.46 (s, 2 H) 7.20-7.41 (m, 5H).

### Step 2: Preparation of N-benzyl-2-methylpent-4-en-2-amine

To a solution of 1-phenyl-N-(propan-2-ylidene)methanamine (1.472 g, 10 mmol) in diethylether (20 mL) was added slowly allymagnesium bromide (1 m solution in tetrahydrofuran, 22 mL) at 0° C. The reaction mixture was stirred at 0° C. for 1 hr and at room temperature for 3 hrs. The mixture was diluted with saturated aqueous ammonium chloride solution and the separated aqueous layer was extracted with diethylether. The combined organic layers were dried over sodium sulfate, filtered off and concentrated under reduced pressure providing crude N-benzyl-2-methylpent-4-en-2-amine (1.75 g), which was directly used at next step without further purification. ¹H NMR (300 MHz, chloroform-d) δ [ppm]: 1.14-1.31 (m, 6H) 2.20-2.40 (m, 2H) 3.71-3.77 (m, 4H) 5.03-5.15 (m, 2H) 5.80-5.90 (m, 1H) 7.20-7.36 (m, 5H).

### Step 3: Preparation of ethyl 2-((benzyl(2-methylpent-4-en-2-yl)amino)methyl)acrylate

To a solution of N-benzyl-2-methylpent-4-en-2-amine (284 mg, 1.5 mmol) in acetonitrile (4 mL) was added powdered potassium carbonate (498 mg, 2.4 mmol) and ethyl 2-(bromomethyl)acrylate (319 mg, 1.65 mmol) and the mixture was stirred at room temperature overnight. The reaction mixture was filtered and the filterate was concentrated under reduced pressure. The residue was purified by column chromatography [silica gel, 24 g, EtOAc/heptane=0/100 to 25/75] providing ethyl 2-((benzyl(2-methylpent-4-en-2-yl)amino)methyl)acrylate (194 mg) as a clear liquid. LCMS (m/z): 302.2 [M+H]+; Rt=0.73 min.

### Step 4: Preparation of ethyl 1-benzyl-6,6-dimethyl-1,2,5,6-tetrahydropyridine-3-carboxylate

To a solution of ethyl 2-((benzyl(2-methylpent-4-en-2-yl)amino)methyl)acrylate (194 mg, 0.644 mmol) in toluene (6.5 mL) under nitrogen atmosphere was added p-toluenesulfonic acid monohydrate (135 mg, 0.708 mmol). The mixture was heated to 50° C. for 30 min, (1,3-bis(2,4,6-trimethylphenyl)-2-(imidazolidinylidene)(dichlorophenylmethylene)-(tricyclohexylphosphine)ruthenium (2nd generation Grubbs catalyst, 27.3 mg) was added and heated was continued at 55° C. for 5 hrs. The mixture was allowed to cool to room temperature, diluted with saturated aqueous sodium carbonate solution (2 mL) and filtered through a pad of celite. The separated organic phase was dried over sodium sulfate, filtered off and concentrated under reduced pressure. The residue was purified by column chromatography [silica gel, 24 g, EtOAc/heptane=10/90 to 25/75] providing ethyl 1-benzyl-6,6-dimethyl-1,2,5,6-tetrahydropyridine-3-carboxylate (117 mg) as a clear liquid. LCMS (m/z): 274.1 [M+H]+; Rt=0.58 min.

### Step 5: Preparation of ethyl 1-benzyl-6,6-dimethylpiperidine-3-carboxylate

To a solution of 1-benzyl-6,6-dimethyl-1,2,5,6-tetrahydropyridine-3-carboxylate (117 mg, 0.428 mmol) in MeOH (5 mL) was added magnesium (turnings, 41.6 mg, 1.712 mmol) and the mixture was vigorously stirred at 33° C. for 5 hrs. The mixture was partitioned between saturated aqueous ammonium chloride solution (20 mL) and diethylether (20 mL). The separated aqueous layer was extracted with diethylether (3×10 mL) and the combined organic layers were dried over sodium sulfate, filtered off and concentrated under reduced pressure providing crude ethyl 1-benzyl-6,6-dimethylpiperidine-3-carboxylate (115 mg) as a light yellow liquid, which was directly used at next step without further purification. LCMS (m/z): 276.2 [M+H]+; Rt=0.59 min.

### Step 6: Preparation of 1-benzyl-6,6-dimethylpiperidine-3-carboxylic acid

A mixture of 1-benzyl-6,6-dimethyl-1,2,5,6-tetrahydropyridine-3-carboxylate (118 mg, 0.428 mmol) and lithium hydroxide (102 mg, 4.28 mmol) in tetrahydrofuran (1 mL), MeOH (1 mL) and water (0.5 mL) was stirred at room temperature overnight. The mixture was acidified with 1N aqueous hydrochloride solution until pH˜5-6 and extracted with EtOAc (5×20 mL). The combined organic layers were dried over sodium sulfate, filtered off and concentrated under reduced pressure providing crude 1-benzyl-6,6-dimethylpiperidine-3-carboxylic acid (55 mg), which was directly used in the next step without further purification. LCMS (m/z): 248.2 [M+H]+; Rt=0.38 min.

### Synthesis of 1-(tert-butoxycarbonyl)-6,6-dimethylpiperidine-3-carboxylic acid

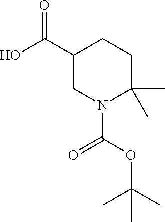

### Step 1: Preparation of methyl 6,6-dimethylpiperidine-3-carboxylate

A mixture of methyl 1-benzyl-6,6-dimethylpiperidine-3-carboxylate (55 mg, 0.210 mmol), ammonium formate (66.3 mg, 1.052 mmol) and Pd/C (10 wt. %, water 50 wt. %, 6 mg) in MeOH (1 mL) was stirred at 70° C. for 30 min. The mixture was allowed to cool to room temperature filtered off to remove Pd/C and solids. The filterate was concentrated in high vacuo providing crude methyl 6,6-dimethylpiperidine-3-carboxylate (36 mg) as a light yellow liquid, which was directly used without further purification. LCMS (m/z): 171.4 [M+H]+; Rt=0.21 min.

### Step 2: Preparation of 6,6-dimethyl-piperidine-1,3-dicarboxylic acid 1-tert-butyl ester 3-methyl ester

To a mixture of methyl 6,6-dimethylpiperidine-3-carboxylate (36.0 mg, 0.21 mmol) and triethylamine (0.088 mL, 0.630 mmol) in tetrahydrofuran (1.5 mL) was added BOC-anhydride (0.059 mL, 0.252 mmol). The reaction mixture was stirred at 35° C. overnight and concentrated under reduced pressure providing crude 6,6-dimethyl-piperidine-1,3-dicarboxylic acid 1-tert-butyl ester 3-methyl ester (61 mg), which was directly used in the next step without further purification.

### Step 3: Preparation of 1-(tert-butoxycarbonyl)-6,6-dimethylpiperidine-3-carboxylic acid

A mixture of 6,6-dimethyl-piperidine-1,3-dicarboxylic acid 1-tert-butyl ester 3-methyl ester (60 mg, 0.221 mmol) and lithium hydroxide (5.30 mg, 0.221 mmol) in tetrahydrofuran (1 mL), MeOH (1 mL) and water (0.5 mL) was stirred overnight at room temperature. The mixture was concentrated under reduced pressure to remove most of the organic solvents. The residue was acidified with 1N aqueous hydrochloride solution until pH˜5 and extracted with EtOAc (2×20 mL). The combined organic layers were dried over sodium sulfate, filtered off and concentrated under reduced pressure providing crude 1-(tert-butoxycarbonyl)-6,6-dimethylpiperidine-3-carboxylic acid (21 mg), which was directly used in the next step without further purification.

### Synthesis of 1-(benzyloxycarbonyl)-6-(trifluoromethyl)piperidine-3-carboxylic acid

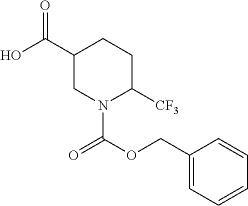

### Step 1: Preparation of ethyl 6-(trifluoromethyl)piperidine-3-carboxylate (mixture of cis and trans isomers)

A mixture of ethyl 6-(trifluoromethyl)nicotinate (2.2 g, 10 mmol), Pd/C (10 wt. %, 100 mg) and platinum(IV)oxide (150 mg, 0.661 mmol) in acetic acid (30 mL) was stirred in a steel bomb under hydrogen atmosphere (200 psi) at 25° C. for 24 hrs. The reaction mixture was filtered through a pad of celites and washed with MeOH (150 mL). The filtrate was concentrated under reduced pressure providing crude ethyl 6-(trifluoromethyl)piperidine-3-carboxylate (776 mg; mixture of cis and trans isomers) as a colorless oil, which was directly used in the next step without further purification. LCMS (m/z): 226.1 [M+H]+; Rt=0.36 min.

### Step 2: Preparation of 6-trifluoromethyl-piperidine-1,3-dicarboxylic acid 1-benzyl ester 3-ethyl ester [mixture of 4 isomers]

To a mixture of crude ethyl 6-(trifluoromethyl)piperidine-3-carboxylate (766 mg, 3.4 mmol) aqueous sodium carbonate solution (10 wt. %, 5 mL) in tetrahydrofuran (15 mL) was added slowly benzylchloroformate (0.583 mL, 4.08 mmol). The reaction mixture was stirred at 25° C. for 24 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 24 g, EtOAc/heptane=0/100 to 30/70] providing a mixture of the cis and trans isomers of 6-trifluoromethyl-piperidine-1,3-dicarboxylic acid 1-benzyl ester 3-ethyl ester (826 mg) as an oil. LCMS (m/z): 316.1 [M+H]+; Rt=1.07 min.

### Step 3: Preparation of 1-(benzyloxycarbonyl)-6-(trifluoromethyl)piperidine-3-carboxylic acid [mixture of 4 isomers]

To 1-benzyl 6-trifluoromethyl-piperidine-1,3-dicarboxylic acid 1-benzyl ester 3-ethyl ester (823 mg, 2.38 mmol) in MeOH (1.8 mL) and water (1.2 mL) was added 6N aqueous sodium hydroxide solution (0.6 mL, 3.6 mmol). The resulting reaction mixture was stirred at 25° C. for 1.5 hrs and concentrated under reduced pressure to a volume of ˜0.5 mL. The mixture was acidified with 1N hydrochloride solution until pH˜4, diluted with EtOAc and stirred for 10 min. The separated organic layer was washed with brine solution, dried over sodium sulfate, filtered off and concentrated under reduced pressure providing 1-(benzyloxycarbonyl)-6-(trifluoromethyl)piperidine-3-carboxylic acid (782 mg, mixture of 4 isomers) as a colorless oil, which was directly used in the next step without further purification. LCMS (m/z): 332.0 [M+H]+; Rt=0.90 min.

### Synthesis of (3R,6R)-/(3S,6S)-1-(benzyloxycarbonyl)-6-ethylpiperidine-3-carboxylic acid and (3R,6S)-/(3R,6S)-1-(benzyloxycarbonyl)-6-ethylpiperidine-3-carboxylic acid

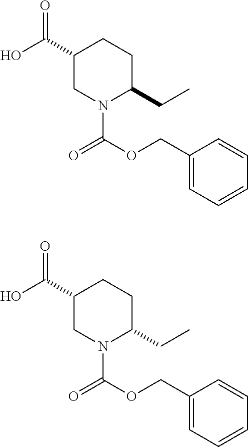

### Step 1: Preparation of methyl 6-ethylnicotinate

To a solution of methyl 6-chloronicotinate (5.0 g, 29.1 mmol), ferric acetylacetonate (1.0 g, 2.83 mmol) in tetrahydrofuran (160 mL) and NMP (1 mL) was added slowly a solution of ethylmagnesium bromide (1M in tetrahydrofuran, 1.09 mL, 7.27 mmol). The reaction mixture was stirred at 25° C. for 3 hrs. The reaction mixture was diluted with saturated aqueous ammonium chloride solution and stirred for additional 30 min. The mixture was diluted with EtOAc, the separated organic layer was washed with saturated aqueous ammonium chloride solution, water and brine. The organic phase was dried over sodium sulfate, filtered off and concentrated under reduced pressure. The residue was purified by column chromatography [silica gel, 80 g, EtOAc/heptane=0/100 to 30/70] providing methyl 6-ethylnicotinate (2.48 g) as an oil. LCMS (m/z): 166.1 [M+H]+; Rt=0.32 min.

### Step 2: Preparation of methyl 6-ethylpiperidine-3-carboxylate (mixture of cis and trans isomers)

A mixture of methyl 6-ethylnicotinate (2.48 g, 15 mmol), Pd/C (10 wt. %, 100 mg) and platinum(IV)oxide (150 mg, 0.661 mmol) in acetic acid (30 mL) was stirred in a steel bomb under hydrogen atmosphere (200 psi) at 25° C. for 16 hrs. The reaction mixture was filtered through a pad of celites and washed with MeOH (150 mL). The filtrate was concentrated under reduced pressure providing crude methyl 6-ethylpiperidine-3-carboxylate (4.45 g; mixture of cis and trans isomers) as a colorless oil, which was directly used in the next step without further purification. LCMS (m/z): 172.1 [M+H]+; Rt=0.31 min.

### Step 3: Preparation of (3R,6S)-/(3S,6R)-6-ethyl-piperidine-1,3-dicarboxylic acid 1-benzyl ester 3-methyl ester [cis isomers] and (3R,6R)-/(3S,6S)-6-ethyl-piperidine-1,3-dicarboxylic acid 1-benzyl ester 3-methyl ester [trans isomers]

To a mixture of crude methyl 6-ethylpiperidine-3-carboxylate (4.5 g, 15 mmol) aqueous sodium carbonate solution (10 wt. %, 30 mL) in tetrahydrofuran (60 mL) was added slowly benzylchloroformate (2.14 mL, 15 mmol). The reaction mixture was stirred at 25° C. for 2 hrs. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 30/70] providing a mixture of the cis isomers (3R,6S)-/(3S,6R)-6-ethyl-piperidine-1,3-dicarboxylic acid 1-benzyl ester 3-methyl ester (3.03 g) as a colorless oil and a mixture of the trans isomers (3R,6R)-/(3S,6S)-6-ethyl-piperidine-1,3-dicarboxylic acid 1-benzyl ester 3-methyl ester (1.23 g) as a solid.

Cis isomers: LCMS (m/z): 306.1 [M+H]+; Rt=1.01 min. Analytical HPLC: Rt=4.15 min.

1H NMR (400 MHz, methanol-d4) δ [ppm]: 0.83 (t, J=6.85 Hz, 3H) 1.49 (d, J=5.87 Hz, 1H) 1.66-1.76 (m, 4H) 1.85-1.93 (m, 1H) 2.38-2.49 (m, J=11.79, 11.79, 4.21, 3.91 Hz, 1H) 2.90 (d, J=1.96 Hz, 1H) 3.67 (s, 3H) 4.16-4.29 (m, 2H) 5.12 (br. s., 2H) 7.28-7.40 (m, 5H).

Trans isomers: LCMS (m/z): 306.1 [M+H]+; Rt=0.98 min. Analytical HPLC: Rt=4.01 min.

1H NMR (400 MHz, methanol-d4) δ [ppm]: 0.83 (t, J=7.43 Hz, 3H) 1.43-1.57 (m, 2 H) 1.71-1.93 (m, 3H) 1.94-2.02 (m, 1H) 2.64 (br. s., 1H) 3.11 (dd, J=14.09, 3.91 Hz, 1H) 3.49-3.69 (m, 3H) 4.11-4.20 (m, 1H) 4.45 (d, J=13.69 Hz, 1H) 5.03-5.19 (m, 2H) 7.19-7.40 (m, 5H).

### Step 3-a: Preparation of (3R,6R)-/(3S,6S)-1-(benzyloxycarbonyl)-5-ethylpiperidine-3-carboxylic acid [trans isomers]

To a mixture of trans isomers (3R,6R)-/(3S,6S)-1-benzyl 3-methyl 6-ethylpiperidine-1,3-dicarboxylate (1.23 g, 3.1 mmol) in MeOH (3 mL) and water (2 mL) was added 6N aqueous sodium hydroxide solution (1.0 mL, 6 mmol). The reaction mixture was stirred at 25° C. for 2.5 hrs and concentrated under reduced pressure to a volume of −2 mL. The mixture was acidified with 1N aqueous hydrochloride solution until pH˜4, diluted with EtOAc and stirred for 10 min. The separated organic layer was washed with brine, dried over sodium sulfate, filtered off and concentrated under reduced pressure providing a mixture of crude (3R,6R)-/(3S,6S)-1-(benzyloxycarbonyl)-6-ethylpiperidine-3-carboxylic acid (1.02 g) as a white solid, which was directly used in the next step without further purification. LCMS (m/z): 292.2 [M+H]+; Rt=0.85 min.

### Step 3-b: Preparation of (3R,6S)-/(3S,6R)-1-(benzyloxycarbonyl)-6-ethylpiperidine-3-carboxylic acid [cis isomers]

To a mixture of cis isomers (3R,6S)-/(3S,6R)-1-benzyl 3-methyl 6-ethylpiperidine-1,3-dicarboxylate (0.92 g, 3.0 mmol) in MeOH (3 mL) and water (2 mL) was added 6N aqueous sodium hydroxide solution (1.0 mL, 6 mmol). The reaction mixture was stirred at 25° C. for 1.5 hrs and concentrated under reduced pressure to a volume of −2 mL. The mixture was acidified with 1N aqueous hydrochloride solution until pH˜4, diluted with EtOAc and stirred for 10 min. The separated organic layer was washed with brine, dried over sodium sulfate, filtered off and concentrated under reduced pressure providing a mixture of crude (3R,6S)-/(3S,6R)-1-(benzyloxycarbonyl)-6-ethylpiperidine-3-carboxylic acid (0.91 g) as an oil, which was directly used in the next step without further purification. LCMS (m/z): 292.1 [M+H]+; Rt=0.87 min.

### Synthesis of (3R,6S)-/(3S,6R)-1-(benzyloxycarbonyl)-6-(methoxymethyl)piperidine-3-carboxylic acid

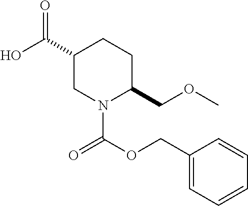

### Step 1: Preparation of methyl 6-(hydroxymethyl)nicotinate

To a mixture of dimethylpyridine-2,5-dicarboxylate (3.08 g, 15.78 mmol) and calcium chloride (7.01 g, 63.1 mmol) in tetrahydrofuran (33 mL) and EtOH (67 mL) was added sodium borohydride (1.493 g, 39.5 mmol) in portions at 0° C. The reaction mixture was stirred at 0° C. for 12 hrs. The mixture was poured into ice/water, was diluted with dichloromethane (400 mL) and stirred vigorously for 15 minutes. The separated organic layer was dried over magnesium sulfate, filtered off and concentrated under reduced pressure providing methyl 6-(hydroxymethyl)nicotinate (1.2 g) as an off white solid, which was directly used in the next step without further purification. LCMS (m/z): 168.0 [M+H]+; Rt=0.26 min

### Step 2: Preparation of methyl 6-(chloromethyl)nicotinate

A mixture of methyl 6-(hydroxymethyl)nicotinate (250 mg, 1.496 mmol) and thionyl chloride (1 mL, 13.70 mmol) in dichloromethane (2 mL) was stirred at 45° C. for 3 hrs and concentrated under reduced pressure. The residue was taken up in dichloromethane (25 mL), sonicated and concentrated under reduced pressure. This was repeated three times and the residue was dried in high vacuo providing of methyl 6-(chloromethyl)nicotinate (266 mg), which was used in the next reaction without further purification. LCMS (m/z): 186.0 [M+H]+; Rt=0.63 min.

### Step 3: Preparation of methyl 6-(methoxymethyl)nicotinate

To a solution of methyl 6-(chloromethyl)nicotinate (250 mg, 1.347 mmol) in MeOH (2 mL) was added sodium methoxide (25 wt. % in MeOH; 1 mL). The mixture was heated at 75° C. for 30 min and concentrated under reduced pressure. The residue was dissolved in EtOAc and the organic layer was washed saturated aqueous sodium bicarbonate solution (3×), dried over magnesium sulfate, filtered off and concentrated under reduced pressure. The residue was purified by column chromatography [silica gel, 12 g, EtOAc/heptane=0/100 to 70/30] providing methyl 6-(methoxymethyl)nicotinate (129 mg). LCMS (m/z): 182.0 [M+H]+; Rt=0.43 min.

### Step 4: Preparation of methyl 6-(methoxymethyl)piperidine-3-carboxylate (mixture of cis and trans isomers)

A mixture of methyl 6-(methoxymethyl)nicotinate (250 mg, 1.380 mmol) and platinum(IV)oxide (100 mg, 0.440 mmol) in acetic acid (10 mL) was stirred in a steel bomb under hydrogen atmosphere (200 psi) at 25° C. for 12 hrs. The reaction mixture was filtered through a pad of celites and washed with dichloromethane (50 mL). The filtrate was concentrated under reduced pressure providing crude methyl 6-(methoxymethyl)piperidine-3-carboxylate (266 mg; mixture of cis and trans isomers) as a colorless oil, which was directly used in the next step without further purification. LCMS (m/z): 188.1 [M+H]+; Rt=0.30 min.

### Step 5: Preparation of (3S,6R)-/(3R,6S)-6-methoxymethyl-piperidine-1,3-dicarboxylic acid 1-benzyl ester 3-methyl ester [trans isomers] and (3R,6R)-/(3S,6S)-6-methoxymethyl-piperidine-1,3-dicarboxylic acid 1-benzyl ester 3-methyl ester [cis isomers]

a mixture of methyl 6-(methoxymethyl)piperidine-3-carboxylate (260 mg, 1.389 mmol) and aqueous sodium carbonate solution (10 wt. %; ˜4 mL) in tetrahydrofuran (4 mL) was added slowly benzylchloroformate (0.297 mL, 2.083 mmol). The reaction mixture was stirred at 25° C. for 1 hr. The mixture was diluted with EtOAc and stirred for additional 10 min. The separated organic layer was dried over magnesium sulfate, filtered off and concentrated under reduced pressure. The residue was purified by column chromatography [silica gel, 12 g, EtOAc/heptane=0/100 to 70/30] providing a mixture of the trans isomers (3S,6R)-/(3R,6S)-6-methoxymethyl-piperidine-1,3-dicarboxylic acid 1-benzyl ester 3-methyl ester (256 mg) and a mixture of the cis isomers (3R,6R)-/(3S,6S)-6-methoxymethyl-piperidine-1,3-dicarboxylic acid 1-benzyl ester 3-methyl ester (200 mg).

Cis isomers: LCMS (m/z): 322.1 [M+H]+; Rt=0.89 min. Analytical HPLC: Rt=4.20 min.

Trans isomers: LCMS (m/z): 322.1 [M+H]+; Rt=0.86 min. Analytical HPLC: Rt=3.98 min.

### Step 6-a: Preparation of (3S,6R)-/(3R,6S)-1-(benzyloxycarbonyl)-6-(methoxymethyl)piperidine-3-carboxylic acid [trans isomers]

To 1-benzyl 3-methyl 6-(methoxymethyl)piperidine-1,3-dicarboxylate (40 mg, 0.124 mmol) in MeOH (3 mL) was added 1N aqueous sodium hydroxide solution (3 mL). The reaction mixture was stirred at 25° C. for 12 hrs and concentrated under reduced pressure to a volume of ˜2 mL. The mixture was acidified with 12N hydrochloride until pH˜4, diluted with EtOAc and stirred for 10 min. The separated organic layer was dried over magnesium sulfate, filtered off and concentrated under reduced pressure providing a mixture of (3S,6R)-/(3R,6S)-1-(benzyloxycarbonyl)-6-(methoxymethyl)piperidine-3-carboxylic acid (35 mg) as a colorless oil, which was directly used in the next step without further purification. LCMS (m/z): 308.1 [M+H]+; Rt=0.73 min.

### Synthesis of (3S,4R)-1-(benzyloxycarbonyl)-4-isopropoxypyrrolidine-3-carboxylic acid

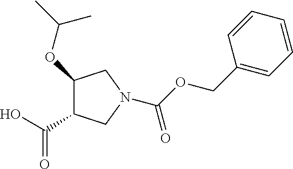

### Step 1: Preparation of (3R,4S)-benzyl 3-isopropoxy-4-vinylpyrrolidine-1-carboxylate

To a solution of (3R,4S)-benzyl 3-hydroxy-4-vinylpyrrolidine-1-carboxylate (3.0 g, 12.13 mmol) in acetonitrile (30 mL) was added 2-iodopropane (20.6 g, 121 mmol) and silver(I)oxide (8.43 g, 36.4 mmol). The mixture was stirred at room temperature for 18 hrs. The solid was filtered off and the filtrate was concentrated under reduced pressure. The residue was purified by column chromatography [silica gel] providing (3R,4S)-benzyl 3-isopropoxy-4-vinylpyrrolidine-1-carboxylate (870 mg). LCMS (m/z): 290.0 [M+H]+; Rt=1.03 min.

### Step 2: Preparation of (3S,4R)-1-(benzyloxycarbonyl)-4-isopropoxypyrrolidine-3-carboxylic acid

A mixture of (3R,4S)-benzyl 3-isopropoxy-4-vinylpyrrolidine-1-carboxylate (550 mg, 1.90 mmol), ruthenium trichloride (496 mg, 1.90 mmol) and sodium periodate (1.63 g, 7.60 mmol) in carbontetrachloride (10 mL), water (10 mL) and acetonitrile (10 mL) were stirred at room temperature overnight. The reaction mixture was diluted with dichloromethane (200 mL) and water (200 mL). The mixture was filtered off and the separated aqueous layer was washed with dichloromethane (2×). All organic layers were combined, dried over sodium sulfate, filtered off and concentrated under reduced pressure. The residue was purified by column chromatography [silica gel, EtOAc/heptane=0/100 to 90/10] providing (3S,4R)-1-(benzyloxycarbonyl)-4-isopropoxypyrrolidine-3-carboxylic acid (350 mg). LCMS (m/z): 308.0 [M+H]+; Rt=0.82 min.

### Synthesis of (3R,5S)-1-(tert-butoxycarbonyl)-5-((2-methoxyethoxy)methyl)pyrrolidine-3-carboxylic acid

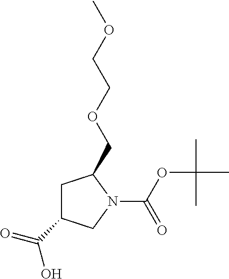

### Step 1: Preparation of (2S,4S)-4-(tert-butyl-diphenyl-silanyloxy)-pyrrolidine-1,2-dicarboxylic acid 1-tert-butyl ester 2-methyl ester

To a solution of (2S,4S)-4-hydroxy-pyrrolidine-1,2-dicarboxylic acid 1-tert-butyl ester 2-methyl ester (2.54 g, 10.25 mmol) in DCM (20 mL) was added the imidazole (1.187 g, 17.43 mmol) followed by tert-butylchlorodiphenylsilane (2.90 mL, 11.28 mmol) at room temperature and the reaction mixture was stirred for 18 hrs. The reaction mixture was filtered and the filtrate was washed with water and brine, dried over sodium sulfate, filtered off and concentrated under reduced pressure providing (2S,4S)-4-(tert-butyl-diphenyl-silanyloxy)-pyrrolidine-1,2-dicarboxylic acid 1-tert-butyl ester 2-methyl ester (4.9 g, 10.09 mmol, 98% yield). LCMS (m/z): 506.2 [M+H]+; Rt=1.46 min.

### Step 2: Preparation of (2S,4S)-tert-butyl 4-(tert-butyldiphenylsilyloxy)-2-(hydroxymethyl)pyrrolidine-1-carboxylate

To a solution of (2S,4S)-4-(tert-butyl-diphenyl-silanyloxy)-pyrrolidine-1,2-dicarboxylic acid 1-tert-butyl ester 2-methyl ester (5.6 g, 11.58 mmol) in tetrahydrofuran (50 mL) was added sodium borohydride (0.876 g, 23.16 mmol) and the mixture was stirred at 70° C. for 4 hrs. The reaction mixture was allowed to cool to room temperature and was diluted with EtOAc (100 mL). The mixture was washed with water, aqueous sodium bicarbonate solution and brine and concentrated under reduced pressure. The residue was purified by column chromatography [silica gel, 40 g, EtOAc/heptane=0/100 to 70/30] providing (2S,4S)-tert-butyl 4-(tert-butyldiphenylsilyloxy)-2-(hydroxymethyl)pyrrolidine-1-carboxylate (3.9 g). LCMS (m/z): 456.2 [M+H]+; Rt=1.30 min.

### Step 3: Preparation of (2S,4S)-tert-butyl 4-(tert-butyldiphenylsilyloxy)-2-((2-methoxyethoxy)methyl)pyrrolidine-1-carboxylate

To a solution of (2S,4S)-tert-butyl 4-(tert-butyldiphenylsilyloxy)-2-(hydroxymethyl)pyrrolidine-1-carboxylate (1.3 g, 2.86 mmol) in tetrahydrofuran (10 mL) was added carefully sodium hydride (60 wt. % in mineral oil, 142 mg, 3.42 mmol) and the mixture was stirred at 25° C. for 1 hr. To the mixture was added bromo ethyl methyl ether (0.714 g, 5.14 mmol) and stirring was continued at 25° C. for 18 hrs. The reaction mixture was diluted with EtOAc, washed with water, saturated aqueous sodium bicarbonate solution and brine and concentrated under reduced pressure. The residue was purified by column chromatography [silica gel] providing (2S,4S)-tert-butyl 4-(tert-butyldiphenylsilyloxy)-2-((2-methoxyethoxy)methyl)pyrrolidine-1-carboxylate (800 mg). LCMS (m/z): 514.2 [M+H]+; Rt=1.41 min.

### Step 4: Preparation of (2S,4S)-tert-butyl 4-hydroxy-2-((2-methoxyethoxy)methyl)-pyrrolidine-1-carboxylate

To a solution of (2S,4S)-tert-butyl 4-(tert-butyldiphenylsilyloxy)-2-((2-methoxyethoxy)methyl)pyrrolidine-1-carboxylate (310 mg, 0.603 mmol) in tetrahydrofuran (5 mL) was added tetrabutylammonium fluoride (316 mg, 1.207 mmol) and the mixture was stirred at 25° C. for 2 hrs. The reaction mixture was diluted with EtOAc (100 mL) and washed with water, brine, dried over sodium sulfate, filtered off and concentrated under reduced pressure. The residue was purified by column chromatography [silica gel, 24 g, EtOAc/heptane=0/100 to 50/50] providing (2S,4S)-tert-butyl 4-hydroxy-2-((2-methoxyethoxy)methyl)pyrrolidine-1-carboxylate (140 mg). LCMS (m/z): 298.1 [M+Na]+; Rt=0.67 min.

### Step 5: Preparation of (2S,4S)-tert-butyl 2-((2-methoxyethoxy)methyl)-4-(tosyloxy)pyrrolidine-1-carboxylate

A mixture of (2S,4S)-tert-butyl 4-hydroxy-2-((2-methoxyethoxy)methyl)pyrrolidine-1-carboxylate (140 mg, 0.508 mmol) and tosyl chloride (291 mg, 1.525 mmol) in pyridine (5 mL) was stirred at 25° C. for 18 hrs. The reaction mixture was diluted with EtOAc (50 mL), washed with water (2×) and brine. The organic layer was dried over sodium sulfate, filtered off and concentrated under reduced pressure. The residue was dissolved in dichloromethane (2 mL) and was purified by column chromatography [silica gel] providing (2S,4S)-tert-butyl 2-((2-methoxyethoxy)methyl)-4-(tosyloxy)pyrrolidine-1-carboxylate (180 mg, LCMS (m/z): 430.1 [M+H]+; Rt=1.06 min.

### Step 6: Preparation of (2S,4R)-tert-butyl 4-cyano-2-((2-methoxyethoxy)methyl)-pyrrolidine-1-carboxylate

To a solution of 2S,4S)-tert-butyl 2-((2-methoxyethoxy)methyl)-4-(tosyloxy)pyrrolidine-1-carboxylate (180 mg, 0.419 mmol) in DMF (2 mL) was added tetrabutylammonium cyanide (343 mg, 1.26 mmol) and the mixture was stirred at 60° C. for 18 hrs. The reaction mixture was diluted with EtOAc (50 mL) and washed with water and brine. The organic layer was dried over sodium sulfate, filtered off and concentrated under reduced pressure. The residue was purified by column chromatography [silica gel] providing (2S,4R)-tert-butyl 4-cyano-2-((2-methoxyethoxy)methyl)pyrrolidine-1-carboxylate (123 mg). LCMS (m/z): 285.1 [M+H]+; Rt=0.82 min.

### Step 7: Preparation of (3R,5S)-1-(tert-butoxycarbonyl)-5-((2-methoxyethoxy)methyl)-pyrrolidine-3-carboxylic acid

A mixture of (2S,4R)-tert-butyl 4-cyano-2-((2-methoxyethoxy)methyl)pyrrolidine-1-carboxylate (123 mg, 0.433 mmol), 6N aqueous sodium hydroxide solution (2 mL, 12 mmol) and EtOH (2 mL) in a closed vial was stirred at 85° C. for 3 hrs. The reaction mixture was allowed to cool to room temperature, acidified with 1N aqueous hydrochloride solution until pH˜5 and extracted with dichloromethane (3×100 mL). The combined organic layers were concentrated under reduced pressure and the residue was dissolved in EtOAc. The organic layer was washed with water, brine, dried over sodium sulfate filtered off and concentrated under reduced pressure. The residue was purified by column chromatography [silica gel] providing (3R,5S)-1-(tert-butoxycarbonyl)-5-((2-methoxyethoxy)methyl)pyrrolidine-3-carboxylic acid (29 mg). LCMS (m/z): 326.0 [M+Na]+; Rt=0.69 min.

### Synthesis of (3R,5S)-/(3S,5R)-1-(benzyloxycarbonyl)-5-methoxypiperidine-3-carboxylic acid and (3R,5R)-/(3S,5S)-1-(benzyloxycarbonyl)-5-methoxypiperidine-3-carboxylic acid

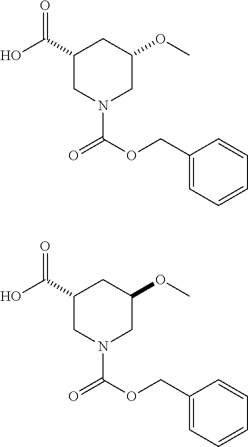

### Step 1: Preparation of methyl 5-methoxypiperidine-3-carboxylate (mixture of cis and trans isomers)

A mixture of methyl 5-methoxynicotinate (1 g, 5.98 mmol), Pd/C (10 wt. %, 90 mg) and platinum(IV)oxide (135 mg, 0.595 mmol) in acetic acid (18 mL) was stirred in a steel bomb under hydrogen atmosphere (200 psi) at 25° C. for 6 hrs. The reaction mixture was filtered through a Celite pad, and washed with MeOH (100 mL). The filtrate was concentrated under reduced pressure providing crude methyl 5-methoxypiperidine-3-carboxylate (1.53 g; mixture of cis and trans isomers) as a colorless oil, which was directly used in the next step without further purification. LCMS (m/z): 174.1 [M+H]+; Rt=0.26 min.

### Step 2: Preparation of (3R,5S)-/(3S,5R)-5-methoxy-piperidine-1,3-dicarboxylic acid 1-benzyl ester 3-methyl ester [cis isomers] and (3R,5R)-/(3S,5S)-5-methoxy-piperidine-1,3-dicarboxylic acid 1-benzyl ester 3-methyl ester [trans isomers]

To a mixture of crude methyl 5-methoxypiperidine-3-carboxylate (1.5 g, 6.06 mmol) aqueous sodium carbonate solution (10 wt. %, 12 mL) in tetrahydrofuran (38 mL) was added slowly benzylchloroformate (1.09 mL, 7.27 mmol). The reaction mixture was stirred at 25° C. for 90 min. The mixture was diluted with EtOAc and stirred for additional 30 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 120 g, EtOAc/heptane=0/100 to 50/50] providing a mixture of the cis isomers (3R,5S)-/(3S,5R)-5-methoxy-piperidine-1,3-dicarboxylic acid 1-benzyl ester 3-methyl ester (441 mg) as colorless oil and a mixture of the cis/trans isomers 5-methoxy-piperidine-1,3-dicarboxylic acid 1-benzyl ester 3-methyl ester (596 mg) as colorless oil.

Cis isomers: LCMS (m/z): 308.1 [M+H]+; Rt=0.89 min. Analytical HPLC: Rt=3.510 min.

Cis/Trans isomers: LCMS (m/z): 308.0 [M+H]+; Rt=0.83 min. Analytical HPLC: Rt=3.516 min.

### Step 3-a: Preparation of (3R,5S)-/(3S,5R)-1-(benzyloxycarbonyl)-5-methoxypiperidine-3-carboxylic acid [cis isomers]

To a mixture of the cis isomers (3R,5S)-/(3S,5R)-5-methoxy-piperidine-1,3-dicarboxylic acid 1-benzyl ester 3-methyl ester (440 mg, 1.43 mmol) in MeOH (1.44 mL) and water (0.96 mL) was added 6N aqueous sodium hydroxide solution (0.48 mL, 2.88 mmol). The reaction mixture was stirred at 25° C. for 1 hr and concentrated under reduced pressure to a volume of ˜0.5 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of (3R,5S)-/(3S,5R)-1-(benzyloxycarbonyl)-5-methoxypiperidine-3-carboxylic acid (323 g) as a white solid, which was directly used in the next step without further purification. LCMS (m/z): 294.0 [M+H]+; Rt=0.71 min.

### Step 3-b: Preparation of 1-(benzyloxycarbonyl)-5-methylpiperidine-3-carboxylic acid [cis/trans isomers]

To a mixture of cis/trans isomers of 5-methoxy-piperidine-1,3-dicarboxylic acid 1-benzyl ester 3-methyl ester (596 mg, 1.94 mmol) in MeOH (1.95 mL) and water (1.3 mL) was added 6N aqueous sodium hydroxide solution (0.65 mL, 3.9 mmol). The reaction mixture was stirred at 25° C. for 2 hrs and concentrated under reduced pressure to a volume of ˜0.5 mL. The mixture was acidified with 1N hydrochloride until pH˜4, diluted with EtOAc and stirred for 10 min. The separated organic layer was washed with brine solution, dried over sodium sulfate, filtered off and concentrated under reduced pressure providing a mixture of cis/trans isomers of 1-(benzyloxycarbonyl)-5-methoxypiperidine-3-carboxylic acid (530 mg) as a colorless oil, which was directly used in the next step without further purification. LCMS (m/z): 294.0 [M+H]+; Rt=0.71 min.

### Example 1

### (R)-Piperidine-3-carboxylic acid [5′-chloro-6-(3-fluoro-benzylamino)-[2,4+]bipyridinyl-2′-yl]-amide

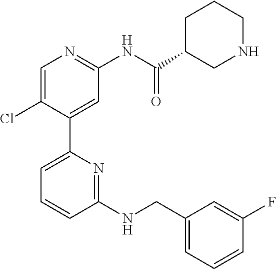

### Step 1: Preparation of (R)-3-[5′-chloro-6-(3-fluoro-benzylamino)-[2,4′]bipyridinyl-2′-ylcarbamoyl]-piperidine-1-carboxylic acid tert-butyl ester

To a solution of (R)-1-(tert-butoxycarbonyl)piperidine-3-carboxylic acid (0.100 g, 0.436 mmol) in dichloromethane (0.70 mL) under argon was added 1-chloro-N,N,2-trimethylprop-1-en-1-amine (0.076 mL, 0.068 g, 0.508 mmol) at 0° C. The mixture was stirred at room temperature for 30 min and added to a solution of 5′-chloro-N6-(3-fluoro-benzyl)-[2,4′]bipyridinyl-6,2′-diamine (0.1194 g, 0.363 mmol) and pyridine (0.041 mL, 0.040 g, 0.508 mmol) in THF (0.70 mL). The reaction mixture was stirred at room temperature for 30 min and diluted with EtOAc (25 mL). The organic phase was washed with saturated aqueous sodium bicarbonate solution (25 mL). The aqueous bicarbonate layer was extracted with EtOAc (2×25 mL). The combined organic layers were washed with brine (1×25 mL), dried over sodium sulfate, filtered off and concentrated under reduced pressure. The residue was purified by column chromatography [silica gel, 40 g, EtOAc/heptane=25/75 to 75/25] providing (R)-3-[5′-chloro-6-(3-fluoro-benzylamino)-[2,4]bipyridinyl-2′-ylcarbamoyl]-piperidine-1-carboxylic acid tert-butyl ester (0.164 g). LCMS (m/z): 540.2 [M+H]+; Rt=0.89 min. ¹H NMR (300 MHz, chloroform-d) 6 [ppm]: 0.88 (t, J=6.59 Hz, 2H) 1.27 (br. s., 3H) 1.47 (s, 9H) 1.69 (s, 4H) 1.88 (t, J=10.70 Hz, 1H) 1.96-2.08 (m, 1H) 2.37-2.53 (m, 1H) 2.92 (t, J=11.14 Hz, 1H) 3.17 (dd, J=13.48, 9.67 Hz, 1H) 3.88 (d, 1H) 4.06-4.20 (m, 1H) 4.55 (d, J=5.86 Hz, 2H) 5.06 (t, J=5.86 Hz, 1H) 6.40 (d, J=8.21 Hz, 1H) 6.91-7.02 (m, 2H) 7.09 (d, J=9.67 Hz, 1H) 7.16 (d, J=7.62 Hz, 1H) 7.28-7.36 (m, 1H) 7.50 (t, J=7.91 Hz, 1H) 8.30 (s, 1H) 8.46 (s, 1H).

### Step 2: Preparation of (R)-piperidine-3-carboxylic acid [5′-chloro-6-(3-fluoro-benzylamino)-[2,4′]bipyridinyl-2′-yl]-amide

To a solution of (R)-3-[5′-chloro-6-(3-fluoro-benzylamino)-[2,4′]bipyridinyl-2′-ylcarbamoyl]-piperidine-1-carboxylic acid tert-butyl ester (0.1639 g, 0.304 mmol) in MeOH (1.26 mL) was added 4N hydrochloride solution in dioxane (6.40 mL, 0.304 mmol). The reaction mixture was stirred at room temperature for 1 hr and concentrated under reduced pressure. The residue was dissolved in saturated aqueous sodium carbonate solution and extracted with dichloromethane (3×50 mL). The combined organic layers were washed with saturated aqueous sodium carbonate solution (1×50 mL) and brine (1×50 mL), dried over sodium sulfate, filtered off and concentrated under reduced pressure. The residue was purified by column chromatography [silica gel, 12 g, dichloromethane/methanol/NEt₃ 100/0/0 to 95/5/1]. Fractions were combined and concentrated under reduced pressure. The residue was dissolved in dichloromethane (25 mL) and washed with saturated aqueous bicarbonate solution (2×25 mL) and water (2×25 mL), dried over sodium sulfate, filtered off and concentrated under reduced pressure. The residue was then dissolved in acetonitrile/water (1/1) and lyophilized providing (R)-piperidine-3-carboxylic acid [5′-chloro-6-(3-fluoro-benzylamino)-[2,4]bipyridinyl-2′-yl]-amide (0.0887 g). LCMS (m/z): 440.1 [M+H]+; Rt=0.66 min.

### Example 2

### Cyclohexanecarboxylic acid [5′-chloro-6-(3-fluoro-benzylamino)-[2,4′]bipyridinyl-2′-yl]-amide

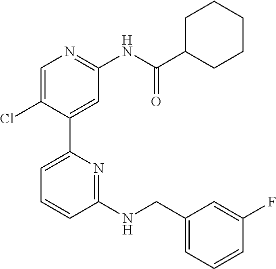

A mixture of cyclohexanecarboxylic acid (36.8 mg, 0.287 mmol), HATU (156 mg, 0.411 mmol) in acetonitrile (1.5 mL) and NMP (0.5 mL) was stirred for ˜60 min. 5′-Chloro-N6-(3-fluorobenzyl)-2,4′-bipyridine-2′,6-diamine (45 mg, 0.137 mmol), dissolved in NMP (0.5 mL), and DIPEA (0.110 mL, 0.630 mmol) were added and the mixture was heated in a sealed tube at 70° C. for ˜16 hrs. The mixture was diluted with EtOAc (˜40 mL). The organic phase was washed with saturated aqueous sodium bicarbonate solution, brine and concentrated under reduced pressure. The residue was dissolved in DMSO (˜2.5 mL), filtered through a syringe filter and purified by HPLC providing cyclohexanecarboxylic acid [5′-chloro-6-(3-fluoro-benzylamino)-[2,4]bipyridinyl-2′-yl]-amide as its trifluoroacetic acid salt (6.0 mg). LCMS (m/z): 439.1 [M+H]+; Rt=0.98 min.

### Example 3

### (R)-Piperidine-3-carboxylic acid {5′-chloro-6-[(tetrahydro-pyran-4-ylmethyl)-amino]-[2,4′]bipyridinyl-2′-yl}-amide

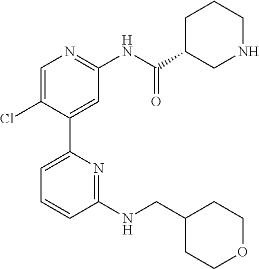

### Step 1: Preparation of (R)-3-{5′-chloro-6-[(tetrahydro-pyran-4-ylmethyl)-amino]-[2,4′]bipyridinyl-2′-ylcarbamoyl}-piperidine-1-carboxylic acid tert-butyl ester

To a solution of (R)-1-(tert-butoxycarbonyl)piperidine-3-carboxylic acid (672 mg, 2.93 mmol) in dichloromethane (5.15 mL) at 0° C. was added 1-chloro-N,N,2-trimethylprop-1-en-1-amine (0.459 mL, 3.47 mmol). The mixture was allowed to stir for 30 min at room temperature. To this mixture was added a solution/suspension of 5′-chloro-N6-((tetrahydro-2H-pyran-4-yl)methyl)-2,4′-bipyridine-2′,6-diamine (850 mg, 2.67 mmol) and pyridine (0.280 mL, 3.47 mmol) in THF (7.5 mL). The mixture was stirred for ˜1 hr at room temperature. The mixture was diluted with EtOAc (˜100 mL) and saturated aqueous sodium bicarbonate solution (˜100 mL). The separated organic layer was washed with saturated aqueous sodium bicarbonate solution and brine and concentrated under reduced pressure. The residue was purified by column chromatography [silica gel, 40 g, 30 min, EtOAc/heptane=30/70 to 60/40] providing (R)-3-{5′-chloro-6-[(tetrahydro-pyran-4-ylmethyl)-amino]-[2,4′]bipyridinyl-2′-ylcarbamoyl}-piperidine-1-carboxylic acid tert-butyl ester (1.38 g). LCMS (m/z): 530.2/532.2 [M+H]+; Rt=0.82 min.

### Step 2: Preparation of (R)-piperidine-3-carboxylic acid {5′-chloro-6-[(tetrahydro-pyran-4-ylmethyl)-amino]-[2,4′]bipyridinyl-2′-yl}-amide

To a solution of (R)-3-{5′-chloro-6-[(tetrahydro-pyran-4-ylmethyl)-amino]-[2,4′]bipyridinyl-2′-ylcarbamoyl}-piperidine-1-carboxylic acid tert-butyl ester (1.30 g, 2.453 mmol) in MeOH (6 mL) was added HCl/dioxane (12 mL, 48.0 mmol) at 0° C. The ice bath was removed and the mixture was stirred for ˜30 min at room temperature. The mixture was concentrated under reduced pressure. The residue was taken up in EtOAc/saturated aqueous sodium bicarbonate solution. The separated organic layer was washed with saturated aqueous sodium bicarbonate solution (1×), dried over sodium sulfate, filtered off and concentrated under reduced pressure. The residue was purified by column chromatography [silica gel, 40 g, dichloromethane/(dichloromethane/methanol/triethylamine; 90/10/0.1)=0/100 to 35/70]. Pure fractions were combined and concentrated under reduced pressure to yield a colorless oil which was stored at ˜−4° C. overnight and then allowed to warm to room temperature. The material was suspended in hexane to yield a white solid, and the hexane was decanted off. The white solid was dried in high vacuo providing (R)-piperidine-3-carboxylic acid {5′-chloro-6-[(tetrahydro-pyran-4-ylmethyl)-amino]-[2,4′]bipyridinyl-2′-yl}-amide (559 mg). Remaining residues were dissolved in dichloromethane and concentrated under reduced pressure providing addition material (260 mg). LCMS (m/z): 430.1 [M+H]+; Rt=0.47 min.

### Example 4

### (S)-Piperidine-3-carboxylic acid [5′-chloro-6-(3-fluoro-benzylamino)-[2,4′]bipyridinyl-2′-yl]-amide

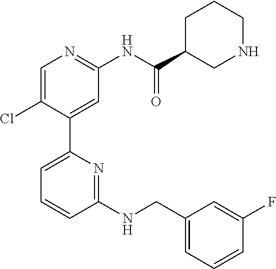

### Step 1: Preparation (S)-tert-butyl 3-(5′-chloro-6-(3-fluorobenzylamino)-2,4′-bipyridin-2′-ylcarbamoyl)piperidine-1-carboxylate

A mixture of (S)-1-(tert-butoxycarbonyl)piperidine-3-carboxylic acid (65.9 mg, 0.287 mmol), HATU (156 mg, 0.411 mmol) in acetonitrile (1.5 mL) and NMP (0.5 mL) was stirred for ˜60 min. 5′-Chloro-N6-(3-fluorobenzyl)-2,4′-bipyridine-2′,6-diamine (45 mg, 0.137 mmol), dissolved in NMP (0.5 mL), and DIPEA (0.110 mL, 0.630 mmol) were added and the mixture was heated in a sealed tube at 70° C. for ˜16 hrs. Additional (S)-1-(tert-butoxycarbonyl)piperidine-3-carboxylic acid (65.9 mg, 0.287 mmol), HATU (156 mg, 0.411 mmol) in acetonitrile (0.8 mL) and NMP (0.200 mL), which was stirred for ˜1 hr, and DIPEA (0.110 mL, 0.630 mmol) were added and heating was continued for ˜20 hrs. The mixture was diluted with EtOAc (˜40 mL). The organic phase was washed with saturated aqueous sodium bicarbonate solution, brine and concentrated under reduced pressure. The residue was dissolved in DMSO (˜1.3 mL), filtered through a syringe filter and purified by HPLC. Fractions were collected and lyophilized providing (S)-3-[5′-chloro-6-(3-fluoro-benzylamino)-[2,4′]bipyridinyl-2′-ylcarbamoyl]-piperidine-1-carboxylic acid tert-butyl ester (26 mg). LCMS (m/z): 540.3/542.2 [M+H]+; Rt=0.95 min.

### Step 2: Preparation of (S)-piperidine-3-carboxylic acid [5′-chloro-6-(3-fluoro-benzylamino)-[2,4′]bipyridinyl-2′-yl]-amide

To a solution of (S)-3-[5′-chloro-6-(3-fluoro-benzylamino)-[2,4′]bipyridinyl-2′-ylcarbamoyl]-piperidine-1-carboxylic acid tert-butyl ester (26 mg) in MeOH (2 mL) was added 4N hydrochloride solution in dioxane (6 mL). The mixture was stirred for ˜30 min at room temperature. The mixture was concentrated under reduced pressure, dissolved in DMSO (1.3 mL), filtered through a syringe filter and purified by HPLC. Pure fractions were collected and lyophilized providing (S)-piperidine-3-carboxylic acid [5′-chloro-6-(3-fluoro-benzylamino)-[2,4′]bipyridinyl-2′-yl]-amide as its trifluoroacetic acid salt (14.6 mg). LCMS (m/z): 440.1/442.2 [M+H]+; Rt=0.77 min.

### Example 9

### 1-Ethyl-piperidine-3-carboxylic acid {5′-chloro-6-[(tetrahydro-pyran-4-ylmethyl)-amino]-[2,4′]bipyridinyl-2′-yl}-amide

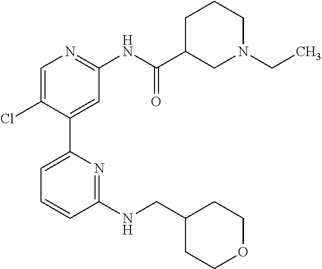

To 1-ethylpiperidine-3-carboxylic acid (26.7 mg, 0.138 mmol) in THF (3 mL) was added DMF (9.72 μL, 0.125 mmol) and slowly oxalyl chloride (0.220 mL, 2.509 mmol). The mixture was stirred at room temperature for 30 min and concentrated under reduced pressure. The residue was diluted with EtOAc (˜1 mL) and the mixture was concentrated under reduced pressure. To the residue was added a solution/suspension of 5′-chloro-N6-((tetrahydro-2H-pyran-4-yl)methyl)-2,4′-bipyridine-2′,6-diamine (40 mg, 0.125 mmol) in THF, followed by the addition of triethylamine (0.175 mL, 1.255 mmol). The mixture was stirred for 30 min, diluted with EtOAc (˜10 mL) and saturated aqueous sodium bicarbonate solution. The separated organic layer was washed with saturated aqueous sodium bicarbonate solution and concentrated under reduced pressure. The residue was dissolved in DMSO (˜2.4 mL), filtered through a syringe filter and purified by HPLC. Pure fractions were collected and lyophilized providing 1-ethyl-piperidine-3-carboxylic acid {5′-chloro-6-[(tetrahydro-pyran-4-ylmethyl)-amino]-[2,4′]bipyridinyl-2′-yl}-amide as its trifluoroacetic acid salt (32 mg). LCMS (m/z): 458.2 [M+H]+; Rt=0.49 min.

### Example 10

### (R)-1-(2-Fluoro-ethyl)-piperidine-3-carboxylic acid {5′-chloro-6-[(tetrahydro-pyran-4-ylmethyl)-amino]-[2,4′]bipyridinyl-2′-yl}-amide

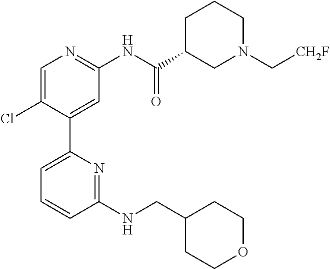

To a mixture of (R)-piperidine-3-carboxylic acid {5′-chloro-6-[(tetrahydro-pyran-4-ylmethyl)-amino]-[2,4′]bipyridinyl-2′-yl}-amide (55 mg, 0.128 mmol) and 1-bromo-2-fluoroethane (0.2 mL, 0.128 mmol) in THF (0.15 mL)/acetonitrile (1.5 mL) was added potassium carbonate (0.1 g, 0.724 mmol). The mixture was heated to 50° C. for ˜3 hrs, allowed to cool to room temperature and diluted with EtOAc (˜15 mL) and water (2 mL). The separated organic layer was concentrated under reduced pressure. The residue was dissolved in DMSO (˜2.4 mL), filtered through a syringe filter and purified by HPLC providing (R)-1-(2-fluoro-ethyl)-piperidine-3-carboxylic acid {5′-chloro-6-[(tetrahydro-pyran-4-ylmethyl)-amino]-[2,4′]bipyridinyl-2′-yl}-amide as its trifluoroacetic acid salt (17.9 mg). LCMS (m/z): 476.2 [M+H]+; Rt=0.49 min.

### Example 11

### (R)-1-(2,2,2-Trifluoro-ethyl)-piperidine-3-carboxylic acid {5′-chloro-6-[(tetrahydro-pyran-4-ylmethyl)-amino]-[2,4′]bipyridinyl-2′-yl}-amide

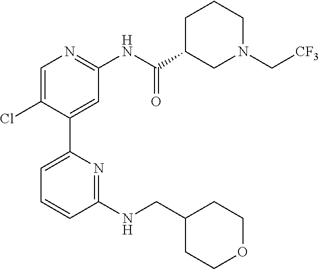

To a mixture of (R)-piperidine-3-carboxylic acid {5′-chloro-6-[(tetrahydro-pyran-4-ylmethyl)-amino]-[2,4′]bipyridinyl-2′-yl}-amide (40 mg, 0.093 mmol) and 2,2,2-trifluoroethyl trifluoromethanesulfonate (32.4 mg, 0.140 mmol) in THF (0.15 mL)/acetonitrile (1.5 mL) was added potassium carbonate (77 mg, 0.558 mmol). The mixture was heated to 50° C. for 90 min. The mixture was then cooled to room temperature and diluted with EtOAc (˜15 mL) and water (2 mL) and the separated organic layer was concentrated under reduced pressure. The resulting residue was dissolved in DMSO (˜2.4 mL), filtered through a syringe filter and purified by HPLC providing (R)-1-(2,2,2-trifluoro-ethyl)-piperidine-3-carboxylic acid {5′-chloro-6-[(tetrahydro-pyran-4-ylmethyl)-amino]-[2,4′]bipyridinyl-2′-yl}-amide as its trifluoroacetic acid salt (29.5 mg). LCMS (m/z): 512.1 [M+H]+; Rt=0.58 min.

### Example 12

### (R)-piperidine-3-carboxylic acid {5′-chloro-6-[(1′,1′-dioxo-hexahydro-1-thiopyran-4-ylmethyl)-amino]-[2,4′]bipyridinyl-2′-yl}-amide

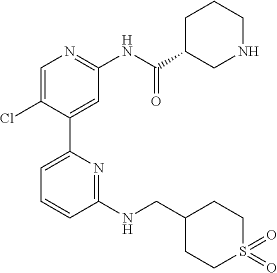

### Step 1: Preparation of (R)-3-{5′-chloro-6-[(1′,1′-dioxo-hexahydro-1-thiopyran-4-ylmethyl)-amino]-[2,4′]bipyridinyl-2′-yl-carbamoyl}-piperidine-1-carboxylic acid tert-butyl ester

To a solution of (R)-1-(tert-butoxycarbonyl)piperidine-3-carboxylic acid (20.62 mg, 0.090 mmol) in dichloromethane (0.5 mL) was added 1-chloro-N,N,2-trimethylprop-1-en-1-amine (14.06 μL, 0.106 mmol) at 0° C. The mixture was stirred at room temperature for 30 min and added to a solution of 5′-chloro-N6-(1′,1′-dioxo-tetrahydro-thiopyran-4-ylmethyl)-[2,4′]bipyridinyl-6,2′-diamine (30 mg, 0.082 mmol) and pyridine (8.60 μL, 0.106 mmol) in THF (1.2 mL). The reaction mixture was stirred at room temperature for 30 min. The reaction mixture was diluted with EtOAc (20 mL), washed with aqueous sodium bicarbonate solution, water and brine and concentrated under reduced pressure. The crude product was purified by column chromatography [silica gel, EtOAc/heptane=0/100 to 100/0]. Fractions were combined and concentrated under reduced pressure providing (R)-3-{5′-chloro-6-[(1′,1′-dioxo-hexahydro-1-thiopyran-4-ylmethyl)-amino]-[2,4′]bipyridinyl-2′-yl-carbamoyl}-piperidine-1-carboxylic acid tert-butyl ester (41 mg). LCMS (m/z): 578.2 [M+H]+; Rt=0.72 min.

### Step 2: Preparation of (R)-piperidine-3-carboxylic acid {5′-chloro-6-[(1′,1′-dioxo-hexahydro-1-thiopyran-4-ylmethyl)-amino]-[2,4′]bipyridinyl-2′-yl}-amide

To a mixture of (R)-3-{5′-chloro-6-[(1′,1′-dioxo-hexahydro-1-thiopyran-4-ylmethyl)-amino]-[2,4′]bipyridinyl-2′-yl-carbamoyl}-piperidine-1-carboxylic acid tert-butyl ester (41 mg, 0.071 mmol) in dichloromethane (1 mL) was added trifluoroacetic acid (546 μL, 7.09 mmol). The mixture was stirred at 25° C. for 1 hr and concentrated under reduced pressure. The residue was dissolved in DMSO and purified by HPLC providing (R)-piperidine-3-carboxylic acid {5′-chloro-6-[(1′,1′-dioxo-hexahydro-1-thiopyran-4-ylmethyl)-amino]-[2,4′]bipyridinyl-2′-yl}-amide as its trifluoroacetic acid salt (39 mg). LCMS (m/z): 478.1 [M+H]+; Rt=0.45 min.

### Example 16

### (R)-1-(2-Methoxy-ethyl)-piperidine-3-carboxylic acid {5′-chloro-6-[(tetrahydro-pyran-4-ylmethyl)-amino]-[2,4′]bipyridinyl-2′-yl}-amide

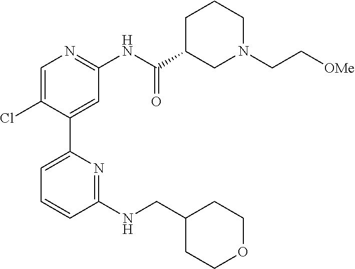

To a mixture of (R)-piperidine-3-carboxylic acid {5′-chloro-6-[(tetrahydro-pyran-4-ylmethyl)-amino]-[2,4′]bipyridinyl-2′-yl}-amide (20 mg, 0.047 mmol) and 1-bromo-2-methoxyethane (38.8 mg, 0.279 mmol) in THF (0.15 mL)/acetonitrile (1.5 mL) was added potassium carbonate (64.3 mg, 0.465 mmol). The mixture was heated to 50° C. for 2 hrs. Additional 1-bromo-2-methoxyethane (38.8 mg, 0.279 mmol) was added and heating was continued for ˜16 hrs. The mixture was cooled to room temperature and diluted with EtOAc (˜15 mL) and water (2 mL). The separated organic layer was concentrated under reduced pressure. The residue was dissolved in DMSO (˜1.2 mL), filtered through a syringe filter and purified by HPLC providing (R)-1-(2-methoxy-ethyl)-piperidine-3-carboxylic acid {5′-chloro-6-[(tetrahydro-pyran-4-ylmethyl)-amino]-[2,4′]bipyridinyl-2′-yl}-amide as its trifluoroacetic acid salt (4.9 mg). LCMS (m/z): 488.2 [M+H]+; Rt=0.50 min.

### Example 17

### (R)-Piperidine-3-carboxylic acid [5′-chloro-6-(cyclohexylmethyl-amino)-[2,4′]bipyridinyl-2′-yl]-amide

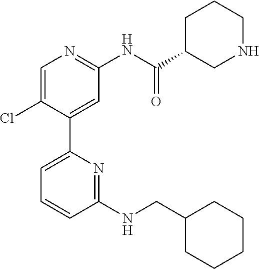

### Step 1: Preparation of (R)-3-[5′-chloro-6-(cyclohexylmethyl-amino)-[2,4′]bipyridinyl-2′-ylcarbamoyl]-piperidine-1-carboxylic acid tert-butyl ester

A solution of (R)-3-(5′-chloro-6-fluoro-[2,4′]bipyridinyl-2′-ylcarbamoyl)-piperidine-1-carboxylic acid tert-butyl ester (17.5 mg, 0.040 mmol) and cyclohexylmethanamine (36.4 mg, 0.322 mmol) in DMSO (0.4 mL) was stirred at 95-100° C. for 20 hrs. The reaction was cooled to room temperature, diluted with EtOAc (12 mL) and washed with saturated aqueous sodium bicarbonate solution (1×) and water (2×) and concentrated under reduced pressure. The crude material of (R)-3-[5′-chloro-6-(cyclohexylmethyl-amino)-[2,4′]bipyridinyl-2′-ylcarbamoyl]-piperidine-1-carboxylic acid tert-butyl ester was directly used in the next step without further purification. LCMS (m/z): 528.3 [M+H]+; Rt=0.96 min.

### Step 2: Preparation of (R)-piperidine-3-carboxylic acid [5′-chloro-6-(cyclohexylmethyl-amino)-[2,4′]bipyridinyl-2′-yl]-amide

To (R)-3-[5′-chloro-6-(cyclohexylmethyl-amino)-[2,4′]bipyridinyl-2′-ylcarbamoyl]-piperidine-1-carboxylic acid tert-butyl ester (0.040 mmol) was added 4N hydrochloride solution in dioxane (0.75 mL, 3.00 mmol) and stirred for 1 hr at room temperature. The mixture was concentrated under reduced pressure, dissolved in DMSO (1 mL), filtered through a syringe filter and purified by HPLC. Fractions were collected and lyophilized providing (R)-piperidine-3-carboxylic acid [5′-chloro-6-(cyclohexylmethyl-amino)-[2,4′]bipyridinyl-2′-yl]-amide as its trifluoroacetic acid salt (8.4 mg). LCMS (m/z): 428.2 [M+H]+; Rt=0.65 min.

### Example 50

### (R)-Pyrrolidine-3-carboxylic acid {5′-chloro-6-[(tetrahydro-pyran-4-ylmethyl)-amino]-[2,4′]bipyridinyl-2′-yl}-amide

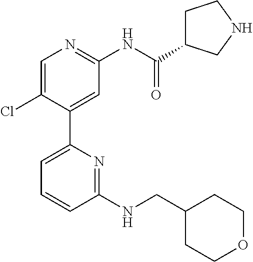

### Step 1: Preparation of (R)-3-{5′-chloro-6-[(tetrahydro-pyran-4-ylmethyl)-amino]-[2,4′]bipyridinyl-2′-ylcarbamoyl}-pyrrolidine-1-carboxylic acid tert-butyl ester

To a solution of (R)-1-(tert-butoxycarbonyl)pyrrolidine-3-carboxylic acid (13.17 mg, 0.061 mmol) in dichloromethane (200 μL) was added 1-chloro-N,N,2-trimethylprop-1-en-1-amine (9.71 μL, 0.073 mmol). The mixture was stirred at room temperature for ˜2 min and added to a solution of 5′-chloro-N6-((tetrahydro-2H-pyran-4-yl)methyl)-2,4′-bipyridine-2′,6-diamine (19.5 mg, 0.061 mmol) and pyridine (4.95 μL, 0.061 mmol) in THF (400 μL). The reaction mixture was stirred at room temperature for 90 min. The mixture was diluted with EtOAc (12 mL) and washed with saturated aqueous sodium bicarbonate solution (1×), brine (1×) and concentrated under reduced pressure. The crude material of (R)-3-{5′-chloro-6-[(tetrahydro-pyran-4-ylmethyl)-amino]-[2,4′]bipyridinyl-2′-ylcarbamoyl}-pyrrolidine-1-carboxylic acid tert-butyl ester was directly used in the next step without further purification. LCMS (m/z): 516.3 [M+H]+; Rt=0.72 min.

### Step 2: Preparation of (R)-pyrrolidine-3-carboxylic acid {5′-chloro-6-[(tetrahydro-pyran-4-ylmethyl)-amino]-[2,4′]bipyridinyl-2′-yl}-amide

To (R)-3-{5′-chloro-6-[(tetrahydro-pyran-4-ylmethyl)-amino]-[2,4′]bipyridinyl-T-ylcarbamoyl}-pyrrolidine-1-carboxylic acid tert-butyl ester was added 4N hydrochloride solution in dioxane (1.5 mL, 6.00 mmol) and stirred for 1 hr at room temperature. The mixture was concentrated under reduced pressure, dissolved in DMSO, filtered through a syringe filter and purified by HPLC. Pure fractions were collected and lyophilized providing (R)-pyrrolidine-3-carboxylic acid {5′-chloro-6-[(tetrahydro-pyran-4-ylmethyl)-amino]-[2,4′]bipyridinyl-2′-yl}-amide as its trifluoroacetic acid salt (18 mg). LCMS (m/z): 416.2 [M+H]+; Rt=0.45 min.

### Example 70

### N-{5′-Chloro-6-[(tetrahydro-pyran-4-ylmethyl)-amino]-[2,4]bipyridinyl-2′-yl}-isobutyramide

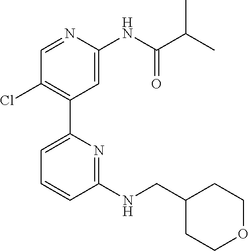

To a mixture of isobutyryl chloride (7.82 mg, 0.073 mmol) and pyridine (5.94 μL, 0.073 mmol) in THF (0.5 mL) was added 5′-chloro-N6-((tetrahydro-2H-pyran-4-yl)methyl)-2,4′-bipyridine-2′,6-diamine (19.5 mg, 0.061 mmol). The reaction mixture was stirred at 24.5° C. for 90 min and concentrated under reduced pressure. The resulting residue was dissolved in DMSO and purified by HPLC providing N-{5′-chloro-6-[(tetrahydro-pyran-4-ylmethyl)-amino]-[2,4′]bipyridinyl-2′-yl}-isobutyramide as its trifluoroacetic acid salt (13 mg). LCMS (m/z): 389.2 [M+H]+; Rt=0.65 min.

### Example 74

### (R)-Piperidine-3-carboxylic acid {5,5′-dichloro-6-[(tetrahydro-pyran-4-ylmethyl)-amino]-[2,4′]bipyridinyl-2′-yl}-amide

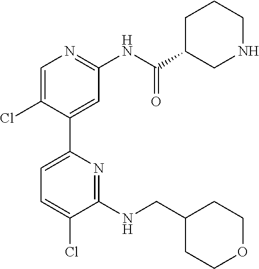

### Step 1: Preparation of (R)-3-{5,5′-dichloro-6-[(tetrahydro-pyran-4-ylmethyl)-amino]-[2,4′]bipyridinyl-2′-ylcarbamoyl}-piperidine-1-carboxylic acid tert-butyl ester

To a solution of (R)-1-(tert-butoxycarbonyl)piperidine-3-carboxylic acid (78 mg, 0.340 mmol) in dichloromethane (0.4 mL) at 0° C. was added 1-chloro-N,N,2-trimethylprop-1-en-1-amine (0.054 mL, 0.408 mmol). The mixture was allowed to stir for 30 min at room temperature. To this mixture was added a solution of 5,5′-dichloro-N6-((tetrahydro-2H-pyran-4-yl)methyl)-2,4′-bipyridine-2′,6-diamine (60 mg, 0.170 mmol) and pyridine (0.033 mL, 0.408 mmol) in THF (0.400 mL). The mixture was stirred for 30 min at room temperature. The mixture was diluted with EtOAc (˜25 mL) and saturated aqueous sodium bicarbonate solution. The separated organic layer was washed with saturated aqueous sodium bicarbonate solution and brine and concentrated under reduced pressure. The residue was dissolved in DMSO, filtered through a syringe filter and purified by HPLC. Fractions were collected and lyophilized providing (R)-3-{5,5′-dichloro-6-[(tetrahydro-pyran-4-ylmethyl)-amino]-[2,4′]bipyridinyl-2′-ylcarbamoyl}-piperidine-1-carboxylic acid tert-butyl ester. LCMS (m/z): 564.3 [M+H]+; Rt=1.20 min.

### Step 2: Preparation of (R)-piperidine-3-carboxylic acid {5,5′-dichloro-6-[(tetrahydro-pyran-4-ylmethyl)-amino]-[2,4′]bipyridinyl-2′-yl}-amide

To a solution of (R)-3-{5,5′-dichloro-6-[(tetrahydro-pyran-4-ylmethyl)-amino]-[2,4′]bipyridinyl-2′-ylcarbamoyl}-piperidine-1-carboxylic acid tert-butyl ester in methanol (2 mL) was added 4N hydrochloride solution in dioxane (4 mL). The mixture was stirred for ˜30 min at room temperature. The mixture was concentrated under reduced pressure, dissolved in DMSO (1.4 mL), filtered through a syringe filter and purified by HPLC. Fractions were collected and lyophilized providing (R)-piperidine-3-carboxylic acid {5,5′-dichloro-6-[(tetrahydro-pyran-4-ylmethyl)-amino]-[2,4′]bipyridinyl-2′-yl}-amide as its trifluoroacetic acid salt (32.7 mg). LCMS (m/z): 464.2 [M+H]+; Rt=0.79 min.

### Example 75

### (R)-Piperidine-3-carboxylic acid {3,5′-dichloro-6-[tetrahydro-pyran-4-ylmethyl)-amino]-[2,4′]bipyridinyl-2′-yl}-amide

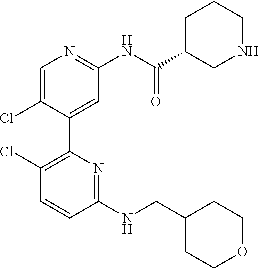

### Step 1: Preparation of (R)-3-{3,5′-dichloro-6-[(tetrahydro-pyran-4-ylmethyl)-amino]-[2,4′]bipyridinyl-2′-ylcarbamoyl}-piperidine-1-carboxylic acid tert-butyl ester

To a solution of (R)-1-(tert-butoxycarbonyl)piperidine-3-carboxylic acid (78 mg, 0.340 mmol) in dichloromethane (0.4 mL) at 0° C. was added 1-chloro-N,N,2-trimethylprop-1-en-1-amine (0.054 mL, 0.408 mmol). The mixture was allowed to stir for 30 min at room temperature. To this mixture was added a solution of 3,5′-dichloro-N6-((tetrahydro-2H-pyran-4-yl)methyl)-2,4′-bipyridine-2′,6-diamine (60 mg, 0.170 mmol) and pyridine (0.033 mL, 0.408 mmol) in THF (0.400 mL). The mixture was stirred for 30 min at room temperature. The mixture was diluted with EtOAc (˜25 mL) and saturated aqueous sodium bicarbonate solution. The separated organic layer was washed with saturated aqueous sodium bicarbonate solution and brine and concentrated under reduced pressure. The residue was dissolved in DMSO, filtered through a syringe filter and purified by HPLC. Fractions were collected and lyophilized providing (R)-3-{3,5′-dichloro-6-[(tetrahydro-pyran-4-ylmethyl)-amino]-[2,4′]bipyridinyl-2′-ylcarbamoyl}-piperidine-1-carboxylic acid tert-butyl ester. LCMS (m/z): 564.2 [M+H]+; Rt=1.04 min.

### Step 2: Preparation of (R)-piperidine-3-carboxylic acid {3,5′-dichloro-6-[(tetrahydro-pyran-4-ylmethyl)-amino]-[2,4′]bipyridinyl-2′-yl}-amide

To a solution of (R)-3-{3,5′-dichloro-6-[(tetrahydro-pyran-4-ylmethyl)-amino]-[2,4′]bipyridinyl-2′-ylcarbamoyl}-piperidine-1-carboxylic acid tert-butyl ester in methanol (2 mL) was added HCl/dioxane (4 mL). The mixture was stirred for ˜30 min at room temperature. The mixture was concentrated under reduced pressure, dissolved in DMSO (1.4 mL), filtered through a syringe filter and purified by HPLC. Pure fractions were collected and lyophilized providing (R)-piperidine-3-carboxylic acid {3,5′-dichloro-6-[(tetrahydro-pyran-4-ylmethyl)-amino]-[2,4′]bipyridinyl-2′-yl}-amide as its trifluoroacetic acid salt (33.3 mg). LCMS (m/z): 464.2 [M+H]+; Rt=0.67 min.

### Example 82

### (R)-1-acetyl-piperidine-3-carboxylic acid {5′-chloro-6-[(tetrahydro-pyran-4-ylmethyl)-amino]-[2,4′]bipyridinyl-2′-yl}-amide

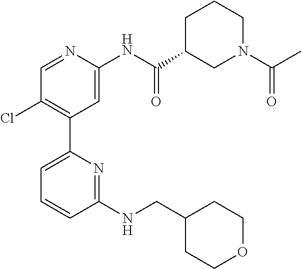

To a solution of (R)-piperidine-3-carboxylic acid {5′-chloro-6-[(tetrahydro-pyran-4-ylmethyl)-amino]-[2,4′]bipyridinyl-2′-yl}-amide (21.5 mg, 0.050 mmol) and pyridine (4.85 μL, 0.060 mmol) in THF (0.6 mL) was added acetic anhydride (5.66 μL, 0.060 mmol). The reaction mixture was stirred at 24.5° C. for 24 hrs and concentrated under reduced pressure. The residue was dissolved in DMSO and purified by HPLC to yield (R)-1-acetyl-piperidine-3-carboxylic acid {5′-chloro-6-[(tetrahydro-pyran-4-ylmethyl)-amino]-[2,4′]bipyridinyl-2′-yl}-amide as its trifluoroacetic acid salt (17.6 mg). LCMS (m/z): 472.3 [M+H]+; Rt=0.57 min.

### Example 116

### (R)-piperidine-3-carboxylic acid {5′-chloro-6-[((S)-2,2-dimethyl-tetrahydro-pyran-4-ylmethyl)-amino]-[2,4′]bipyridinyl-2′-yl}-amide or (R)-piperidine-3-carboxylic acid {5′-chloro-6-[((R)-2,2-dimethyl-tetrahydro-pyran-4-ylmethyl)-amino]-[2,4′]bipyridinyl-2′-yl}-amide

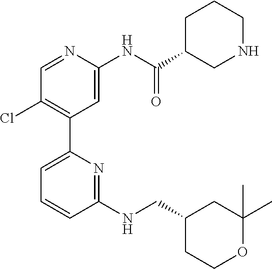

### Step 1: Preparation of (R)-3-{5′chloro-6-[(((S)-2,2-dimethyl-tetrahydro-pyran-4-ylmethyl)-amino]-[2,4′]bipyridinyl-2′-ylcarbamoyl}-piperidine-1-carboxylic acid tert-butyl ester or (R)-3-{5′chloro-6-[((R)-2,2-dimethyl-tetrahydro-pyran-4-ylmethyl)-amino]-[2,4′]bipyridinyl-2′-ylcarbamoyl}-piperidine-1-carboxylic acid tert-butyl ester

A solution of ((R)-1-(tert-butoxycarbonyl)piperidine-3-carboxylic acid (169 mg, 0.74 mmol), and 1-chloro-N,N,2-trimethylprop-1-en-1-amine (0.10 mL, 0.74 mmol) in dichloromethane (2 mL) was added slowly into the solution of 5′chloro-N6-((S)-2,2-dimethyl-tetrahydro-pyran-4-ylmethyl)-[2,4′]bipyridinyl-6,2′-diamine or 5′chloro-N-6-((R)-2,2-dimethyl-tetrahydro-pyran-4-ylmethyl)-[2,4′]bipyridinyl-6,2′-diamine (Intermediate CR1—Fraction 2; 183 mg, 0.53 mmol) and pyridine (55 μL, 0.686 mmol) in THF (3.5 mL). The reaction mixture was stirred at 25° C. for 4 hrs. The mixture was diluted with EtOAc and stirred for additional 10 min. The separated organic layer was washed with saturated aqueous sodium bicarbonate solution, water and brine. The organic phase was dried over sodium sulfate, filtered off and concentrated under reduced pressure. The residue was purified by column chromatography [silica gel, 12 g, EtOAc/heptane=0/100 to 60/40] providing (R)-3-{5′chloro-6-[((S)-2,2-dimethyl-tetrahydro-pyran-4-ylmethyl)-amino]-[2,4′]bipyridinyl-2′-ylcarbamoyl}-piperidine-1-carboxylic acid tert-butyl ester or (R)-3-{5′chloro-6-[((R)-2,2-dimethyl-tetrahydro-pyran-4-ylmethyl)-amino]-[2,4′]bipyridinyl-2′-ylcarbamoyl}-piperidine-1-carboxylic acid tert-butyl ester (305 mg) as solid. LCMS (m/z): 558.3 [M+H]+; Rt=0.82 min.

### Step 2: Preparation of (R)-piperidine-3-carboxylic acid {5′-chloro-6-[((S)-2,2-dimethyl-tetrahydro-pyran-4-ylmethyl)-amino]-[2,4′]bipyridinyl-2′-yl}-amide or (R)-piperidine-3-carboxylic acid {5′-chloro-6-[((R)-2,2-dimethyl-tetrahydro-pyran-4-ylmethyl)-amino]-[2,4′]bipyridinyl-2′-yl}-amide

To solution of (R)-3-{5′chloro-6-[((S)-2,2-dimethyl-tetrahydro-pyran-4-ylmethyl)-amino]-[2,4′]bipyridinyl-2′-ylcarbamoyl}-piperidine-1-carboxylic acid tert-butyl ester or (R)-3-{5′chloro-6-[((R)-2,2-dimethyl-tetrahydro-pyran-4-ylmethyl)-amino]-[2,4′]bipyridinyl-2′-ylcarbamoyl}-piperidine-1-carboxylic acid tert-butyl ester, from Step 1 above, (305 mg, 0.546 mmol) in methanol (0.35 mL) was added 4N hydrochloride solution in dioxane (5 mL, 20 mmol). The yellow reaction solution was stirred at 25° C. for 1 hr. The reaction mixture was concentrated under reduced pressure and the residue was purified by using reversed phase liquid chromatography. Fractions were lyophilized to dryness, the residue was diluted with ethyl acetate. The organic layer was washed with saturated aqueous sodium bicarbonate solution and brine, dried over sodium sulfate, filtered off and concentrated under reduced pressure providing (R)-piperidine-3-carboxylic acid {5′-chloro-6-[((S)-2,2-dimethyl-tetrahydro-pyran-4-ylmethyl)-amino]-[2,4′]bipyridinyl-2′-yl}-amide or (R)-piperidine-3-carboxylic acid {5′-chloro-6-[((R)-2,2-dimethyl-tetrahydro-pyran-4-ylmethyl)-amino]-[2,4′]bipyridinyl-2′-yl}-amide as a solid (176 mg). LCMS (m/z): 458.2 [M+H]+; Rt=0.51 min.

### Example 121

### (3S,4S)-4-hydroxy-pyrrolidine-3-carboxylic acid {5′-chloro-6-[(tetrahydro-pyran-4-ylmethyl)-amino]-[2,4′]bipyridinyl-2′-yl}-amide

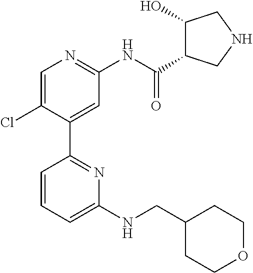

### Step 1: Preparation of (3S,4S)-3-(tert-butyl-diphenyl-silanyloxy)-4-{5′-chloro-6-[(tetrahydro-pyran-4-ylmethyl)-amino]-[2,4′]bipyridinyl-2′-ylcarbamoyl}-pyrrolidine-1-carboxylic acid benzyl ester

To a solution of (3S,4S)-1-(benzyloxycarbonyl)-4-(tert-butyldiphenylsilyloxy)-pyrrolidine-3-carboxylic acid (513 mg, 1.02 mmol) in dichloromethane (1 mL) was added 1-chloro-N,N,2-trimethylprop-1-en-1-amine (178 mg, 1.333 mmol) at 0° C. The mixture was stirred for 30 min at room temperature and added slowly into the solution of 5′-chloro-N6-((tetrahydro-2H-pyran-4-yl)methyl)-2,4′-bipyridine-2′,6-diamine (250 mg, 0.784 mmol) and pyridine (127 μL, 1.568 mmol) in THF (1 mL). The reaction mixture was stirred at room temperature for 1 hr and was concentrated under reduced pressure. The residue was purified by column chromatography [silica gel] providing (3S,4S)-3-(tert-butyl-diphenyl-silanyloxy)-4-{5′-chloro-6-[(tetrahydro-pyran-4-ylmethyl)-amino]-[2,4′]bipyridinyl-2′-ylcarbamoyl}-pyrrolidine-1-carboxylic acid benzyl ester (216 mg). LCMS (m/z): 804.2 [M+H]+; Rt=1.14 min.

### Step 2: Preparation of (3S,4S)-3-{5′-chloro-6-[(tetrahydro-pyran-4-ylmethyl)-amino]-[2,4′]bipyridinyl-2′-ylcarbamoyl}-4-hydroxy-pyrrolidine-1-carboxylic acid benzyl ester

To a solution of (3S,4S)-3-(tert-butyl-diphenyl-silanyloxy)-4-{5′-chloro-6-[(tetrahydro-pyran-4-ylmethyl)-amino]-[2,4′]bipyridinyl-2′-ylcarbamoyl}-pyrrolidine-1-carboxylic acid benzyl ester (200 mg, 0.249 mmol) in THF (5 mL) was added tetrabutylammonium fluoride (65.0 mg, 0.249 mmol) and the mixture was stirred at 25° C. for 2 hrs. The mixture was concentrated under reduced pressure and the residue was dissolved in EtOAc (50 mL). The organic solution was washed with water and brine, dried over sodium sulfate, filtered off and concentrated under reduced pressure. The residue was purified by column chromatography [silica gel] providing (3S,4S)-3-{5′-chloro-6-[(tetrahydro-pyran-4-ylmethyl)-amino]-[2,4′]bipyridinyl-2′-ylcarbamoyl}-4-hydroxy-pyrrolidine-1-carboxylic acid benzyl ester (110 mg). LCMS (m/z): 566.2 [M+H]+; Rt=0.68 min.

### Step 3: Preparation of (3S,4S)-4-hydroxy-pyrrolidine-3-carboxylic acid {5′-chloro-6-[(tetrahydro-pyran-4-ylmethyl)-amino]-[2,4′]bipyridinyl-2′-yl}-amide

A solution of (3S,4S)-3-{5′-chloro-6-[(tetrahydro-pyran-4-ylmethyl)-amino]-[2,4′]bipyridinyl-2′-ylcarbamoyl}-4-hydroxy-pyrrolidine-1-carboxylic acid benzyl ester (80 mg, 0.141 mmol) in ethanol (10 mL) was purged with hydrogen for 30 min and Pd/C (10 wt. %, 3.01 mg) was added. The mixture was stirred under hydrogen atmosphere (˜1 atm, balloon) at 25° C. for 1 hr and filtered through a plug of celites. The filtrate was concentrated under reduced pressure and the residue was purified by HPLC. Fractions were collected and lyophilized providing (3S,4S)-4-hydroxy-pyrrolidine-3-carboxylic acid {5′-chloro-6-[(tetrahydro-pyran-4-ylmethyl)-amino]-[2,4′]bipyridinyl-2′-yl}-amide (30 mg) as its trifluoroacetic acid salt. LCMS (m/z): 432.1 [M+H]+; Rt=0.44 min.

### Example 127

### (3R,6R)-/(3S,6S)-6-methyl-piperidine-3-carboxylic acid {5′-chloro-6-[(tetrahydro-pyran-4-ylmethyl)-amino]-[2,4′]bipyridinyl-2′-yl}-amide (racemic mixture of trans isomers)

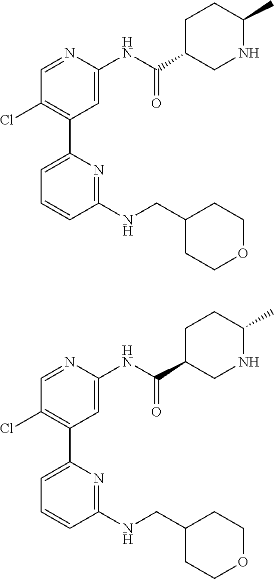

### Step 1: Preparation of (2R,5R)-/(2S,5S)-5-{5′-chloro-6-[(tetrahydro-pyran-4-ylmethyl)-amino]-[2,4′]′bipyridinyl-2′-ylcarbamoyl}-2-methyl-piperidine-1-carboxylic acid benzyl ester (racemic mixture of trans isomers)

A solution of (3R,6R)-/(3S,6S)-1-(benzyloxycarbonyl)-6-methylpiperidine-3-carboxylic acid (532 mg, 1.73 mmol), and 1-chloro-N,N,2-trimethylprop-1-en-1-amine (0.25 mL, 1.88 mmol) in dichloromethane (6 mL) was added slowly into the solution of 5′-chloro-N6-((tetrahydro-2H-pyran-4-yl)methyl)-2,4′-bipyridine-2′,6-diamine (500 mg, 1.57 mmol) and pyridine (0.15 ml, 1.88 mmol) in THF (12 mL). The reaction solution was stirred at 25° C. for 4 hrs. The reaction solution was diluted with EtOAc and stirred for additional 10 min. The separated organic layer was washed with saturated aqueous sodium bicarbonate solution and brine. The organic phase was dried over sodium sulfate, filtered off and concentrated under reduced pressure. The residue was purified by column chromatography [silica gel, 40 g, EtOAc/heptane=10/90 to 60/40] providing (2R,5R)-/(2S,5S)-5-{5′-chloro-6-[(tetrahydro-pyran-4-ylmethyl)-amino]-[2,4′]bipyridinyl-2′-ylcarbamoyl}-2-methyl-piperidine-1-carboxylic acid benzyl ester (racemic mixture of trans isomers, 667 mg) as a solid. LCMS (m/z): 578.4 [M+H]+; Rt=0.83 min.

### Step 2: Preparation of (3R,6R)-/(3S,6S)-6-methyl-piperidine-3-carboxylic acid {5′-chloro-6-[(tetrahydro-pyran-4-ylmethyl)-amino]-[2,4′]bipyridinyl-2′-yl}-amide [trans isomers]

A mixture of (2R,5R)-/(2S,5S)-5-{5′-chloro-6-[(tetrahydro-pyran-4-ylmethyl)-amino]-[2,4′]bipyridinyl-2′-ylcarbamoyl}-2-methyl-piperidine-1-carboxylic acid benzyl ester (667 mg, 1.15 mmol) and Pd/C (10 wt. %, 246 mg, 0.231 mmol) in THF (25 mL) was stirred under hydrogen atmosphere (1 atm, balloon) at 25° C. for 18 hrs. The reaction mixture was filtered through a pad of celites and washed with EtOAc (500 mL). The filtrate was concentrated under reduced pressure and the residue was purified by column chromatography [silica gel, 40 g, dichloromethane/methanol/triethylamine=90/5/0 to 90/10/0.01]. Fractions were concentrated under reduced pressure and the residue was dissolved in ethyl acetate. The organic phase was washed with saturated aqueous sodium bicarbonate solution and brine. The organic phase was dried over sodium sulfate, filtered off and concentrated under reduced pressure providing (3R,6R)-/(3S,6S)-6-methyl-piperidine-3-carboxylic acid {5′-chloro-6-[(tetrahydro-pyran-4-ylmethyl)-amino]-[2,4′]bipyridinyl-2′-yl}-amide (425 mg). LCMS (m/z): 444.3 [M+H]+; Rt=0.48 min.

### Example 146 and Example 147

### (3R,6R)-6-methyl-piperidine-3-carboxylic acid {5′-chloro-6-[(tetrahydro-pyran-4-ylmethyl)-amino]-[2,4′]bipyridinyl-2′-yl}-amide and (3S,6S)-6-methyl-piperidine-3-carboxylic acid {5′-chloro-6-[(tetrahydro-pyran-4-ylmethyl)-amino]-[2,4]bipyridinyl-2′-yl}-amide

The two trans isomers of the racemic mixture in Example 127 were separated by chiral resolution, and the specific stereochemistry of each of the isomers was not conclusively determined. Conditions for chiral resolution are provided in Table A below.

### Example 195

### 6,6-Dimethyl-N-(6-(((tetrahydro-2H-pyran-4-yl)methyl)amino)-2,4′-bipyridin-2′-yl)piperidine-3-carboxamide

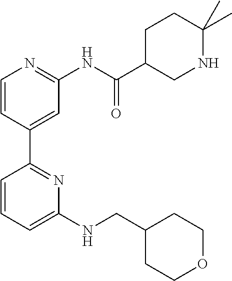

### Step 1: Preparation of 1-benzyl-6,6-dimethyl-piperidine-3-carboxylic acid {5′-chloro-6-[(tetrahydro-pyran-4-ylmethyl)-amino]-[2,4′]bipyridinyl-2′-yl}-amide

To 1-benzyl-6,6-dimethylpiperidine-3-carboxylic acid (50.4 mg, 0.204 mmol) in dichloromethane (1 mL) was added 1-chloro-N,N,2-trimethylprop-1-en-1-amine (29.1 mg, 0.222 mmol) and the mixture was stirred at room temperature for 30 min. To the mixture was added a solution of the 5′-chloro-N6-((tetrahydro-2H-pyran-4-yl)methyl)-2,4′-bipyridine-2′,6-diamine (59 mg, 0.185 mmol) and pyridine (18 μL, 0.222 mmol) in THF (1 mL). The reaction mixture was stirred at room temperature overnight and concentrated under reduced pressure. The residue was dissolved in dichloromethane (1.5 mL) and purified by column chromatography [silica gel, 12 g, EtOAc/heptane=0/100 to 25/75] providing 1-benzyl-6,6-dimethyl-piperidine-3-carboxylic acid {5′-chloro-6-[(tetrahydro-pyran-4-ylmethyl)-amino]-[2,4′]bipyridinyl-2′-yl}-amide (21 mg) as a white solid. LCMS (m/z): 548.4 [M+H]+; Rt=0.60 min.

### Step 2: Preparation of 6,6-dimethyl-piperidine-3-carboxylic acid {6-[(tetrahydro-pyran-4-ylmethyl)-amino]-[2,4] bipyridinyl-2′-yl}-amide

A mixture of 1-benzyl-6,6-dimethyl-piperidine-3-carboxylic acid {5′-chloro-6-[(tetrahydro-pyran-4-ylmethyl)-amino]-[2,4′]bipyridinyl-2′-yl}-amide (20 mg, 0.036 mmol), Pd/C (10 wt. %, ˜50 wt. % water, 6 mg) and ammonium formate (10.35 mg, 0.18 mmol) in MeOH (1 mL) was heated at 72° C. for 1 hr. The reaction mixture was cooled to room temperature, filtered off and the solids were washed with methanol (2×). The filtrate was concentrated under reduced pressure and the residue was purified by HPLC. Fractions were collected and lyophilized providing 6,6-dimethyl-piperidine-3-carboxylic acid {6-[(tetrahydro-pyran-4-ylmethyl)-amino]-[2,4′]bipyridinyl-2′-yl}-amide (4 mg) as its trifluoroacetic acid salt. LCMS (m/z): 424.4 [M+H]+; Rt=0.47 min.

### Example 301

### (R)-Piperidine-3-carboxylic acid {5′-chloro-5-[(tetrahydro-pyran-4-ylmethyl)-amino]-[3,4′]bipyridinyl-2′-yl}-amide

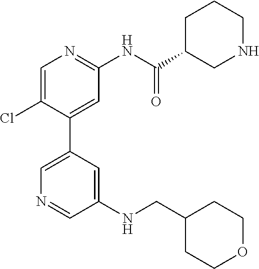

### Step 1: Preparation of (R)-3-{5′-chloro-5-[(tetrahydro-pyran-4-ylmethyl)-amino]-[3,4′]bipyridinyl-2′-ylcarbamoyl}-piperidine-1-carboxylic acid tert-butyl ester

A mixture of (R)-1-(tert-butoxycarbonyl)piperidine-3-carboxylic acid (124 mg, 0.540 mmol), HATU (293 mg, 0.772 mmol) in acetonitrile (1.5 mL) and NMP (0.5 mL) was stirred for ˜1 hr. 5′-Chloro-N5-((tetrahydro-2H-pyran-4-yl)methyl)-3,4′-bipyridine-2′,5-diamine (82 mg, 0.257 mmol), dissolved in NMP (0.5 mL), and DIPEA (0.207 mL, 1.183 mmol) were added and the mixture was heated in a sealed tube at 70° C. for ˜16 hrs. The mixture was diluted with EtOAc (˜40 mL). The organic phase was washed with saturated aqueous sodium bicarbonate solution, brine and concentrated under reduced pressure. The residue was dissolved in DMSO (˜2.5 mL), filtered through a syringe filter and purified by HPLC. Fractions were lyophilized providing (R)-3-{5′-chloro-5-[(tetrahydro-pyran-4-ylmethyl)-amino]-[3,4]bipyridinyl-2′-ylcarbamoyl}-piperidine-1-carboxylic acid tert-butyl ester (45 mg). LCMS (m/z): 530.3/532.2 [M+H]+; Rt=0.76 min.

### Step 2: Preparation of (R)-piperidine-3-carboxylic acid {5′-chloro-5-[(tetrahydro-pyran-4-ylmethyl)-amino]-[3,4′]bipyridinyl-2′-yl}-amide

To a solution of (R)-3-{5′-chloro-5-[(tetrahydro-pyran-4-ylmethyl)-amino]-[3,4]bipyridinyl-2′-ylcarbamoyl}-piperidine-1-carboxylic acid tert-butyl ester (42.5 mg) in MeOH (2 mL) was added 4N hydrochloride solution in dioxane (6 mL). The mixture was stirred for ˜30 min at room temperature. The mixture was concentrated under reduced pressure, dissolved in DMSO (˜2.6 mL), filtered through a syringe filter and purified by HPLC. Fractions were collected and lyophilized (R)-piperidine-3-carboxylic acid {5′-chloro-5-[(tetrahydro-pyran-4-ylmethyl)-amino]-[3,4]bipyridinyl-2′-yl}-amide as its trifluoroacetic acid salt (32.7 mg). LCMS (m/z): 430.1/432.2 [M+H]+; Rt=0.51 min.

### Example 302

### (R)-Piperidine-3-carboxylic acid {6,5′-dichloro-5-[(tetrahydro-pyran-4-ylmethyl)-amino]-[3,4′]bipyridinyl-2′-yl}-amide

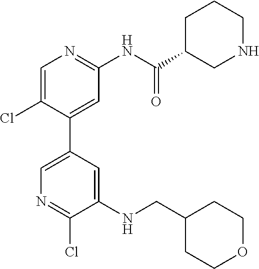

### Step 1: Preparation of (R)-3-{6,5′-dichloro-5-[(tetrahydro-pyran-4-ylmethyl)-amino]-[3,4′]bipyridinyl-2′-ylcarbamoyl}-piperidine-1-carboxylic acid tert-butyl ester

A mixture of (R)-1-(tert-butoxycarbonyl)piperidine-3-carboxylic acid (75.0 mg, 0.327 mmol), HATU (178 mg, 0.467 mmol) in acetonitrile (1.5 mL) and NMP (0.500 mL) was stirred for ˜60 min. 5′,6-Dichloro-N-5-((tetrahydro-2H-pyran-4-yl)methyl)-3,4′-bipyridine-2′,5-diamine (55 mg, 0.156 mmol), dissolved in NMP (0.5 mL), and DIPEA (0.125 mL, 0.716 mmol) were added and the mixture was heated in a sealed tube at 70° C. for ˜16 hrs. Additional (R)-1-(tert-butoxycarbonyl)piperidine-3-carboxylic acid (75.0 mg, 0.327 mmol), HATU (178 mg, 0.467 mmol) in acetonitrile (0.8 mL) and NMP (0.200 mL), which was stirred for ˜1 hr, and DIPEA (0.125 mL, 0.716 mmol) were added and heating was continued for ˜20 hrs. The mixture was diluted with EtOAc (˜40 mL). The organic phase was washed with saturated aqueous sodium bicarbonate solution, brine and concentrated under reduced pressure. The residue was dissolved in DMSO (˜2.5 mL), filtered through a syringe filter and purified by HPLC. Pure fractions were collected and lyophilized providing (R)-3-{6,5′-dichloro-5-[(tetrahydro-pyran-4-ylmethyl)-amino]-[3,4]bipyridinyl-2′-ylcarbamoyl}-piperidine-1-carboxylic acid tert-butyl ester (23 mg). LCMS (m/z): 564.3/566.2 [M+H]+; Rt=1.07 min.

### Step 2: Preparation of (R)-piperidine-3-carboxylic acid {6,5′-dichloro-5-[(tetrahydro-pyran-4-ylmethyl)-amino]-[3,4′]bipyridinyl-2′-yl}-amide

To a solution of (R)-3-{6,5′-dichloro-5-[(tetrahydro-pyran-4-ylmethyl)-amino]-[3,4]bipyridinyl-2′-ylcarbamoyl}-piperidine-1-carboxylic acid tert-butyl ester (20.5 mg) in MeOH (2 mL) was added 4N hydrochloride solution in dioxane (6 mL). The mixture was stirred for ˜30 min at room temperature. The mixture was concentrated under reduced pressure, dissolved in DMSO (1.3 mL), filtered through a syringe filter and purified by HPLC. Fractions were collected and lyophilized (R)-piperidine-3-carboxylic acid {6,5′-dichloro-5-[(tetrahydro-pyran-4-ylmethyl)-amino] [3,4′]bipyridinyl-2′-yl}-amide as its trifluoroacetic acid salt (11.8 mg). LCMS (m/z): 464.2/466.1 [M+H]+; Rt=0.70 min.

Table A provides chiral separation details for a mixture of stereoisomers. The absolute stereochemistry of the two stereoisomers, in a given mixture is known, absolute stereochemistry was not conclusively assigned for each separated stereoisomer.

TABLE A

| | | | Chiral | |
|---|---|---|---|---|
| | | | column for | |
| | | Chiral column | quality | |
| Example | | for separation/ | control/ | Retention |
| No. | Compound Structure | conditions | conditions | time |
| 14 | 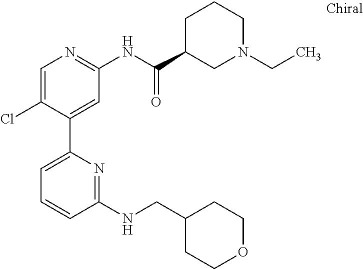 | AD-H column; 16 mg/2 mL EtOH; heptane:EtOH = 90:10; 20 mL/min, 310 psi | AD-H column; heptane:EtOH = 90:10; 1 mL/min | 6.9 min |
| 56 | 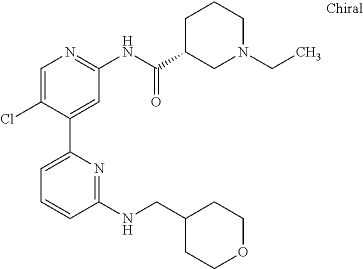 | | AD-H column; heptane:EtOH = 90:10; 1 mL/min | 11.7 min |
| 78 | 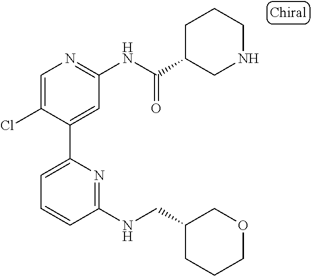 | AD-H column; 30 mg/3 mL EtOH; heptane:EtOH = 90:10; 20 mL/min, 330 psi | AD-H column; heptane:EtOH = 80:20; 1 mL/min | 10.9 min |
| 79 | 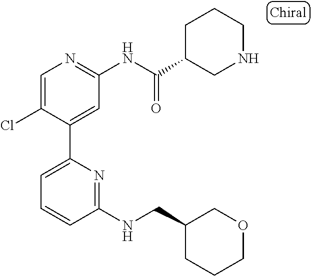 | | AD-H column; heptane:EtOH = 80:20; 1 mL/min | 12.7 min |
| 115 | 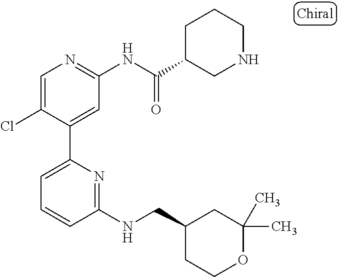 | AD column; 63 mg/5 mL IPA; heptane:IPA = 85:15; 20 mL/min, 320 psi | AD-H column; heptane:IPA = 85:15; 1 mL/min | 10.7 min |
| 116 | 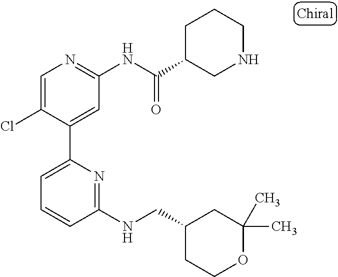 | | AD-H column; heptane:IPA = 85:15; 1 mL/min | 15.6 min |
| 117 | 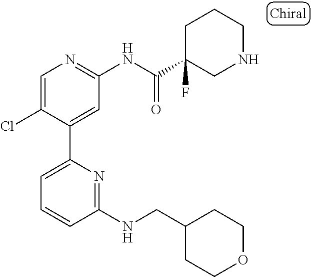 | AD column; 19 mg/2 mL IPA; heptane:IPA = 80:20; 20 mL/min; 330 psi | AD-H column; heptane:IPA = 75:25; 1 mL/min | 9.1 min |
| 118 | 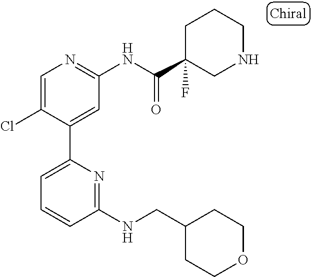 | | AD column; heptane:IPA = 75:25; 1 mL/min | 19.4 min |
| 128 | 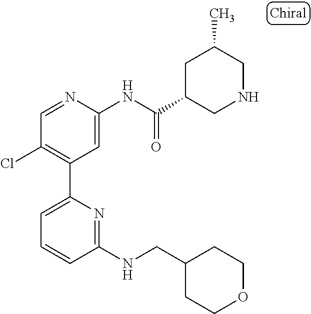 | IA column; 56 mg/4 mL EtOH; heptane:EtOH = 85:15; 15 mL/min, 830 psi | IA column; heptane:EtOH = 80:20; 1 mL/min | 8.0 min |
| 129 | 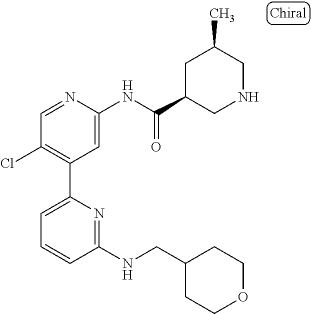 | | IA column; heptane:EtOH = 80:20; 1 mL/min | 14.9 min |
| 130 | 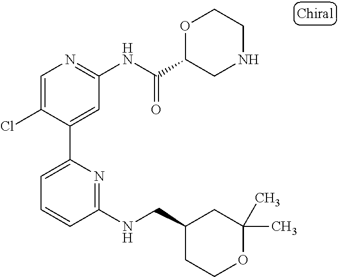 | OJ column; 11 mg/2 mL EtOH; heptane:EtOH = 85:15; 15 mL/min; 810 psi | IA column; heptane:EtOH = 85:15; 1 mL/min | 13.8 min |
| 131 | 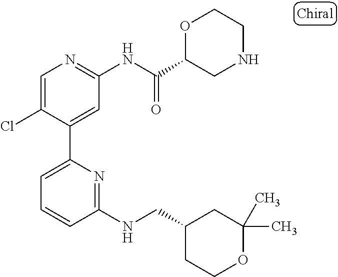 | | IA column; heptane:EtOH = 85:15; 1 mL/min | 19.4 min |
| 141 | 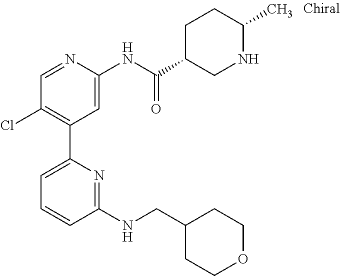 | AD column; 93 mg/6 mL EtOH; heptane:EtOH = 90:10; 20 mL/min, 300 psi | AD column; heptane:IPA = 80:20; 1 mL/min | 4.9 min |
| 142 | 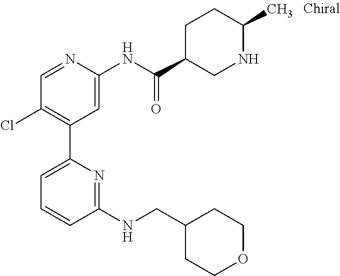 | | AD column; heptane:IPA = 80:20; 1 mL/min | 6.7 min |
| 146 | 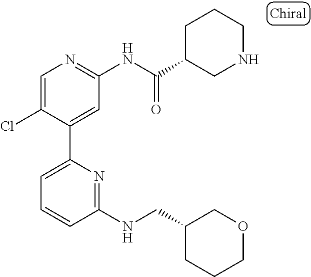 | AD column; 98 mg/8 mL EtOH; heptane:EtOH = 85:15; 20 mL/min, 282 psi | AD-H column; heptane:EtOH = 85:15; 1 mL/min | 7.4 min |
| 147 | 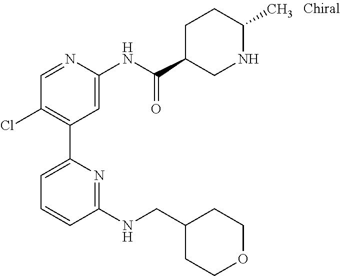 | | AD-H column; heptane:EtOH = 85:15; 1 mL/min | 14.3 min |
| 155 | 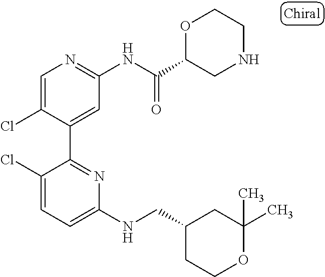 | AD column, 33 mg/3 mL EtOH; heptane:EtOH = 85:15; 20 mL/min; 300 psi | AD-H column; heptane:EtOH = 85:15; 1 mL/min | 8.5 min |
| 156 | 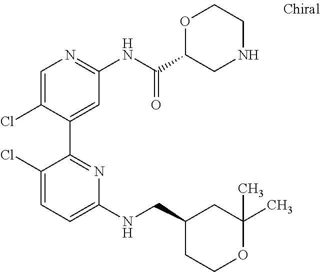 | | AD-H column; heptane:EtOH = 85:15; 1 mL/min | 9.7 min |
| 162 | 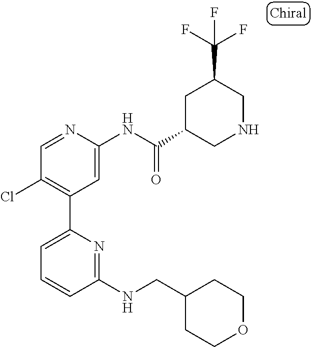 | OD column; 33 mg/3 mL IPA; heptane:IPA = 90:10; 20 mL/min, 486 psi | OD column; heptane:IPA = 90:10; 1 mL/min | 8.5 min |
| 163 | 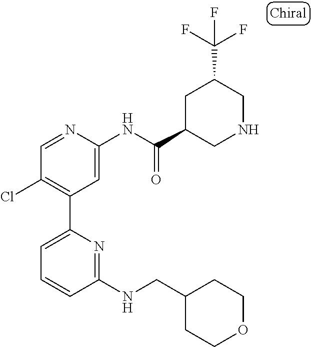 | | OD column; heptane:IPA = 90:10; 1 mL/min | 15.5 min |
| 172 | 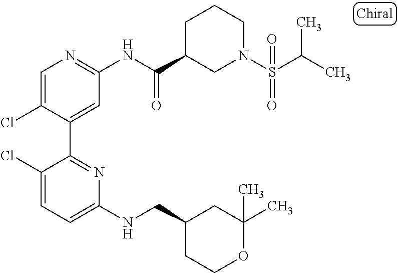 | AD column, 10 mg/2 mL IPA; heptane:IPA = 80:20; 20 mL/min; 340 psi | AD-H column; heptane:IPA = 75:25; 1 mL/min | 10.6 min |
| 173 | 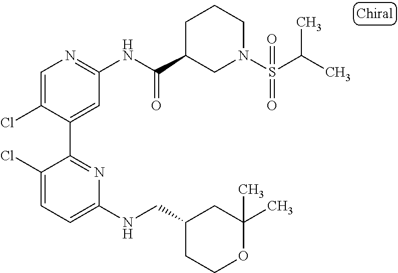 | | AD-H column; heptane:IPA = 75:25; 1 mL/min | 14.4 min |
| 174 | 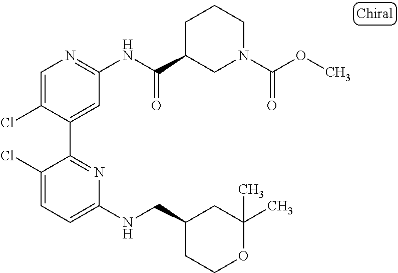 | AD column, 17 mg/3 mL IPA; heptane:IPA = 90:10; 20 mL/min; 300 psi | AD-H column; heptane:IPA = 85:15; 1 mL/min | 9.0 min |
| 175 | 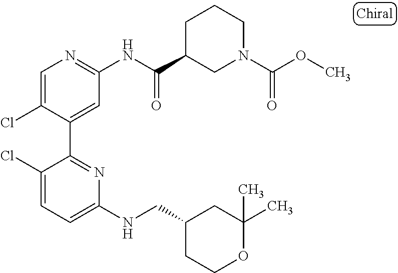 | | AD-H column; heptane:IPA = 85:15; 1 mL/min | 11.7 min |
| 176-Cbz | 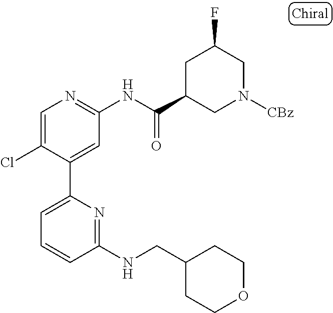 | IA column; 210 mg/10 mL EtOH; heptane:EtOH = 75:25; 12 mL/min; 738 psi | IA column; heptane/EtOH; 60:40; 1 mL/min | 5.5 min |
| 177-Cbz | 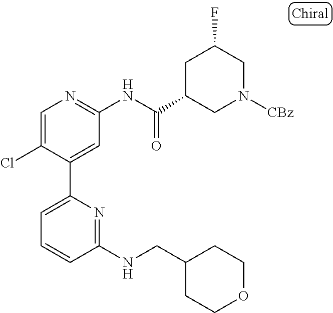 | | IA column; heptane/EtOH; 60:40; 1 mL/min | 7.6 min |
| 178-Cbz | 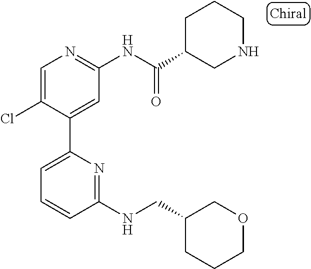 | IA column; 62 mg/4 mL EtOH; heptane:EtOH = 70:30; 12 mL/min; 800 psi | IA column; heptane:EtOH = 60:40; 1 mL/min | 6.3 min |
| 179-Cbz | 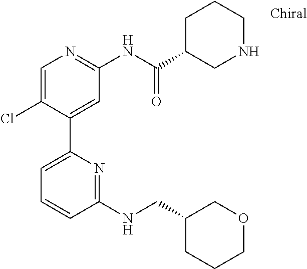 | | IA column; heptane:EtOH = 60:40; 1 mL/min | 12.6 min |
| 182 | 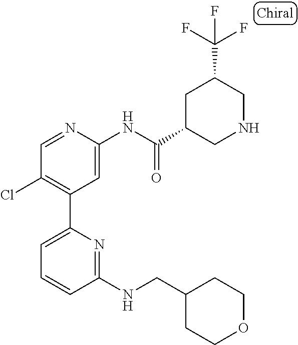 | IA column; 140 mg/40 mL EtOH/Me0H (3:1); heptane:EtOH = 70:30; 12 mL/min, 804 psi | IA column; heptane:EtOH = 70:30; 1 mL/min | 3.7 min |
| 183 | 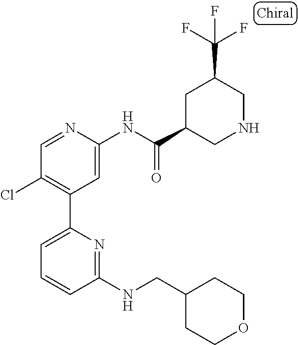 | | IA column; heptane:EtOH = 70:30; 1 mL/min | 7.9 min |
| 189 | 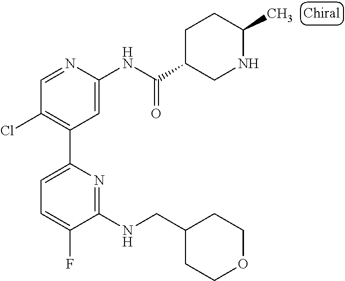 | AD column; 20 mg/4 mL EtOH; heptane:EtOH = 90:10; 20 mL/min, 318 psi | AD-H column; heptane:EtOH = 85:15; 1 mL/min | 6.2 min |
| 190 | 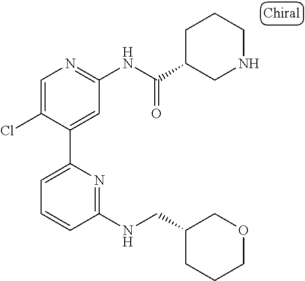 | | AD-H column; heptane:EtOH = 85:15; 1 mL/min | 10.7 min |
| 196 | 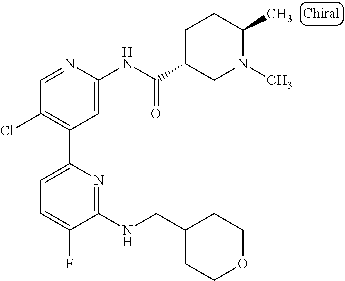 | AD column; 20 mg/4 mL EtOH; heptane:EtOH = 90:10; 20 mL/min; 318 psi | AD-H column; heptane:EtOH = 85:15; 1 mL/min | 3.9 min |
| 197 | 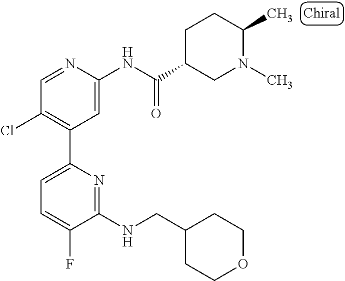 | | AD-H column; heptane:EtOH = 85:15; 1 mL/min | 4.4 min |
| 211 | 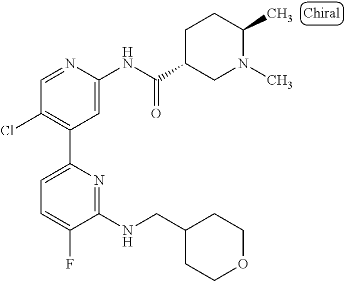 | AD column; 90 mg/6 mL IPA; heptane:IPA = 70:30; 20 mL/min; 400 psi | AD-H column; heptane:IPA = 70:30; 1 mL/min | 8.4 min |
| 212 | 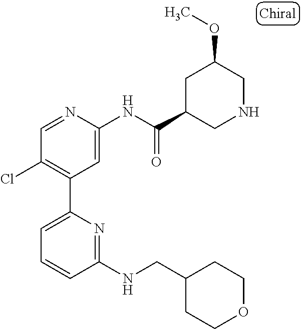 | | AD-H column; heptane:IPA = 70:30; 1 mL/min | 21.1 min |
| 213 | 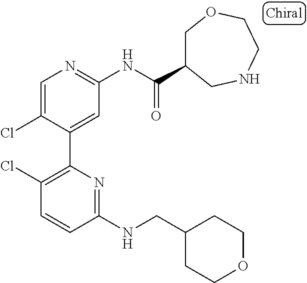 | AD column; 25 mg/4 mL EtOH; heptane:EtOH = 60:40; 20 mL/min; 430 psi | AD-H column; heptane:EtOH = 60:40; 1 mL/min | 7.1 min |
| 214 | 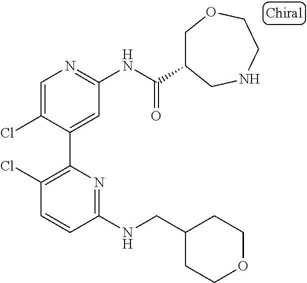 | | AD-H column; heptane:EtOH = 60:40; 1 mL/min | 12.1 min |
| 215 | 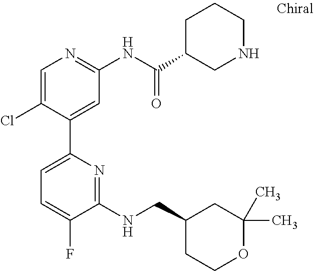 | AD column; 90 mg/6 mL IPA; heptane:IPA = 85:15; 20 mL/min; 330 psi | AD-H column; heptane:IPA = 80:20; 1 mL/min | 7.8 min |
| 216 | 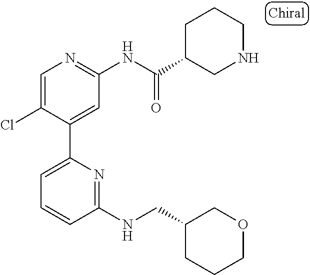 | | AD-H column; heptane:IPA = 80:20; 1 mL/min | 13.5 min |
| 222 | 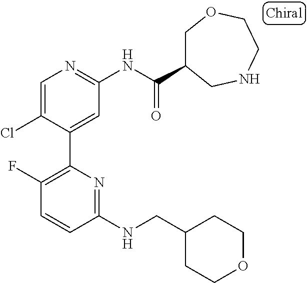 | AD column, 12 mg/3 mL EtOH, heptane:EtOH = 60:40; 20 mL/min; 440 psi | AD-H column; heptane:EtOH = 60:40; 1 mL/min | 7.1 min |
| 223 | 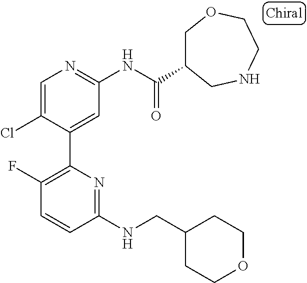 | | AD-H column; heptane:EtOH = 60:40; 1 mL/min | 9.8 min |
| 231 | 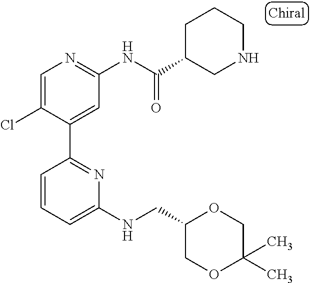 | OJ column; 27 mg/4 mL EtOH; heptane:EtOH = 85:15; 20 mL/min, 690 psi | OJ-H column; heptane:EtOH = 80:20; 1 mL/min | 11.0 min |
| 232 | 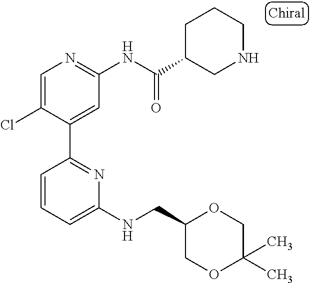 | | OJ-H column; heptane:EtOH = 80:20; 1 mL/min | 7.3 min |
| 233 | 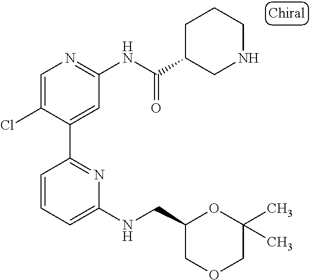 | AD column; 22 mg/4 mL IPA; heptane:IPA = 80:20; 20 mL/min, 340 psi | AD-H column; heptane:IPA = 75:25; 1 mL/min | 8.1 min |
| 234 | 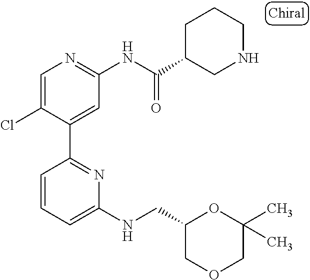 | | AD-H column; heptane:IPA = 75:25; 1 mL/min | 8.9 min |
| 235 | 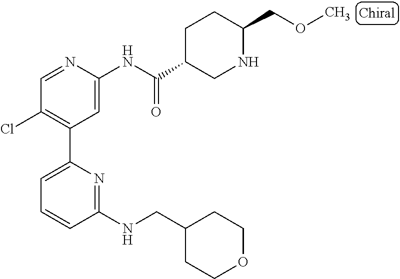 | IA column; 20 mg/2.5 mL EtOH; heptane:ethanol 80:20; 12 mL/min | IA column; heptane:EtOH = 75:25; 1 mL/min | 4.1 min |
| 236 | 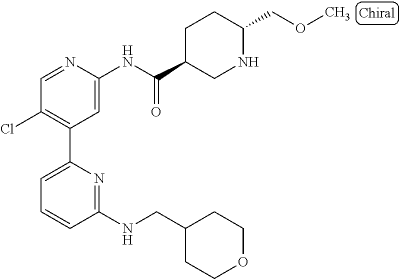 | | IA column; heptane:EtOH = 75:25; 1 mL/min | 7.4 min |
| 240 | 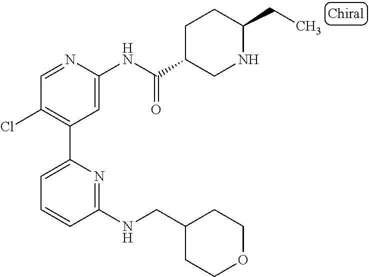 | AD column; 122 mg/10mL EtOH; heptane:EtOH = 70:30; 20 mL/min, 390 psi | AD-H column; heptane:EtOH = 60:40; 1 mL/min | 2.6 min |
| 241 | 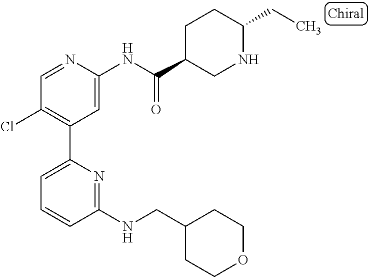 | | AD-H column; heptane:EtOH = 60:40; 1 mL/min | 4.2 min |
| 242 | 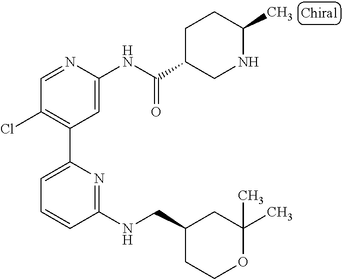 | AD column; 80 mg/8 mL IPA; heptane:IPA = 80:20; 20 mL/min, 310 psi | AD-H column; heptane:IPA = 75:25; 1 mL/min | 3.4 min |
| 243 | 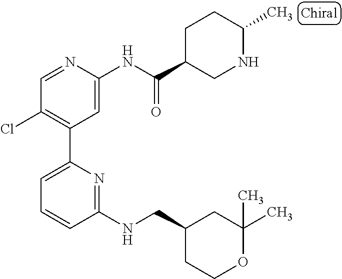 | | AD-H column; heptane:IPA = 75:25; 1 mL/min | 8.2 min |
| 244 | 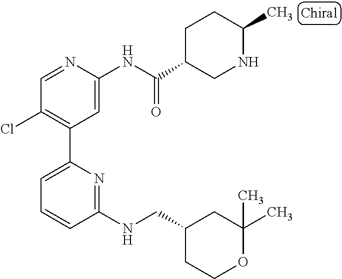 | AD column; 69 mg/6 mL EtOH; heptane:EtOH = 80:20; 20 mL/min, 310 psi | AD-H column; heptane:EtOH = 75:25; 1 mL/min | 3.0 min |
| 245 | 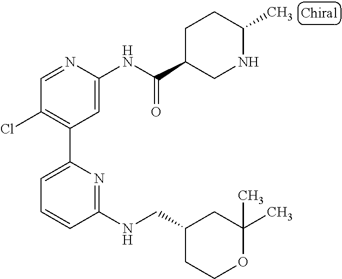 | | AD-H column; heptane:EtOH = 75:25; 1 mL/min | 5.6 min |
| 256 | 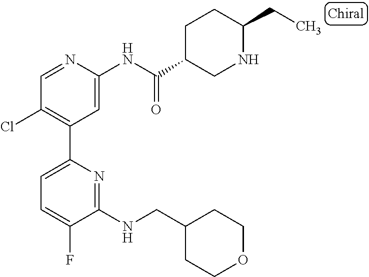 | AD column; 119 mg/8 mL EtOH; heptane:EtOH = 70:30; 20 mL/min, 400 psi | AD-H column; heptane:EtOH = 70:30; 1 mL/min | 3.1 min |
| 257 | 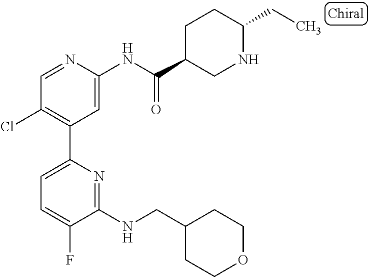 | | AD-H column; heptane:EtOH = 70:30; 1 mL/min | 5.2 min |
| 262 | 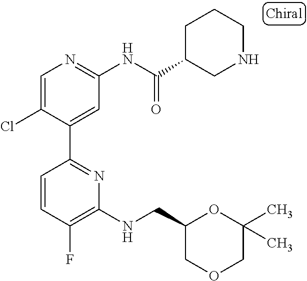 | 100 mg/3 mL EtOH, SFC; IC column; 15 mL/min; CO₂:EtOH = 83:17 + 0.1% DEA | SFC; IC column; 5 mL/min; CO₂:EtOH = 83:17 + 0.1% DEA | 7.4 min |
| 263 | 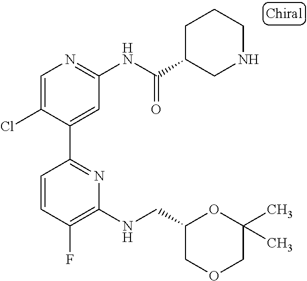 | | SFC; IC column; 5 mL/min; CO₂:EtOH = 83:17 + 0.1% DEA | 8.3 min |
| 265 | 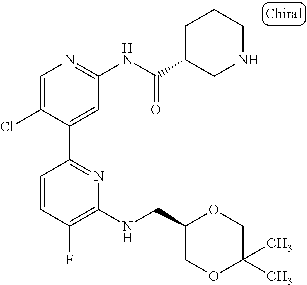 | 100 mg/5 mL EtOH, SFC; OJ column; 15 mL/min; CO₂:EtOH = 88:12 + 0.1% DEA | SFC; OJ column; 5 mL/min; CO₂:EtOH = 90:10 + 0.1% DEA | 5.4 min |
| 264 | 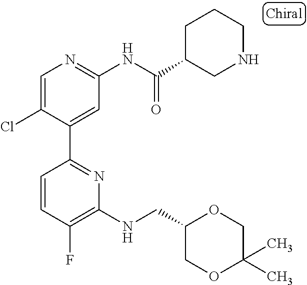 | | SFC; OJ column; 5 mL/min; CO₂:EtOH = 90:10 + 0.1% DEA | 7.3 min |

Tables I and II provide a list of compounds that were prepared using the procedures outlined above, and by using the appropriate starting materials.

TABLE I

| Example | | | Retention | |
|---|---|---|---|---|
| No. | Structure | M + H | Time [min] | Name |
| 1 | 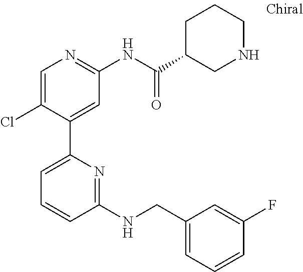 | 440.1 | 0.66 | (R)-Piperidine-3- carboxylic acid [5′- chloro-6-(3-fluoro- benzylamino)- [2,4′]bipyridinyl-2′-yl]- amide |
| 2 | 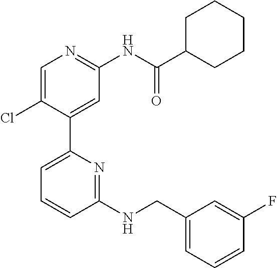 | 439.1 | 0.77 | Cyclohexanecarboxylic acid [5′-chloro-6-(3- fluoro-benzylamino)- [2,4′]bipyridinyl-2′-yl]- amide |
| 3 | 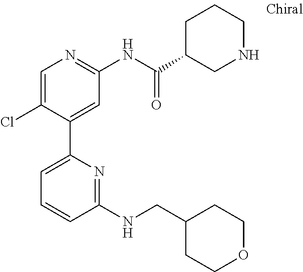 | 430.1 | 0.72 | (R)-Piperidine-3- carboxylic acid {5′- chloro-6-[(tetrahydro- pyran-4-ylmethyl)- amino]- [2,4′]bipyridinyl-2′-yl}- amide |
| 4 | 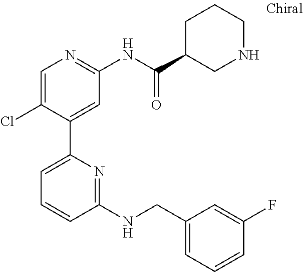 | 440.1 | 0.77 | (S)-Piperidine-3- carboxylic acid [5′- chloro-6-(3-fluoro- benzylamino)- [2,4′]bipyridinyl-2′-yl]- amide |
| 5 | 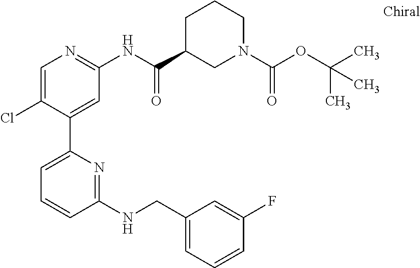 | 540.3 | 0.95 | (S)-3-[5′-Chloro-6-(3- fluoro-benzylamino)- [2,4′]bipyridinyl-2′- ylcarbamoyl]- piperidine-1-carboxylic acid tert-butyl ester |
| 6 | 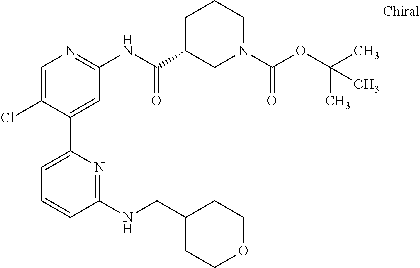 | 564.3 | 0.85 | (R)-3-{5′-Chloro-6- [(tetrahydro-pyran-4- ylmethyl)-amino]- [2,4′]bipyridinyl-2′- ylcarbamoyl}- piperidine-1-carboxylic acid tert-butyl ester |
| 7 | 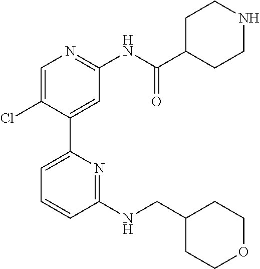 | 430.1 | 0.51 | Piperidine-4-carboxylic acid {5′-chloro-6- [(tetrahydro-pyran-4- ylmethyl)-amino]- [2,4′]bipyridinyl-2′-yl}- amide |
| 8 | 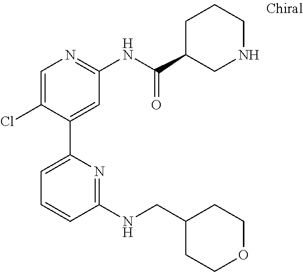 | 430.2 | 0.42 | (S)-Piperidine-3- carboxylic acid {5′- chloro-6-[(tetrahydro- pyran-4-ylmethyl)- amino]- [2,4′]bipyridinyl-2′-yl}- amide |
| 9 | 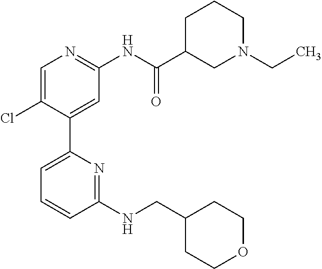 | 458.2 | 0.49 | 1-Ethyl-piperidine-3- carboxylic acid {5′- chloro-6-[(tetrahydro- pyran-4-ylmethyl)- amino]- [2,4′]bipyridinyl-2′-yl}- amide |
| 10 | 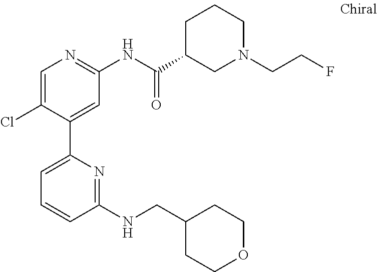 | 476.2 | 0.49 | (R)-1-(2-Fluoro-ethyl)- piperidine-3-carboxylic acid {5′-chloro-6- [(tetrahydro-pyran-4- ylmethyl)-amino]- [2,4′]bipyridinyl-2′-yl}- amide |
| 11 | 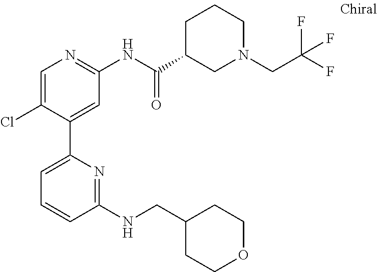 | 512.1 | 0.58 | (R)-1-(2,2,2-Trifluoro- ethyl)-piperidine-3- carboxylic acid {5′- chloro-6-[(tetrahydro- pyran-4-ylmethyl)- amino]- [2,4′]bipyridinyl-2′-yl}- amide |
| 12 | 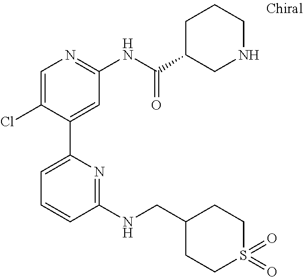 | 478.1 | 0.45 | (R)-Piperidine-3- carboxylic acid {5′- chloro-6-[(1′,1′-dioxo- hexahydro-1-thiopyran- 4-ylmethyl)-amino]- [2,4′]bipyridinyl-2′-yl}- amide |
| 13 | 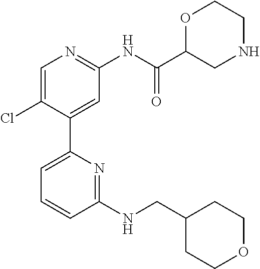 | 432.1 | 0.49 | Morpholine-2- carboxylic acid {5′- chloro-6-[(tetrahydro- pyran-4-ylmethyl)- amino]- [2,4′]bipyridinyl-2′-yl}- amide |
| 14 | 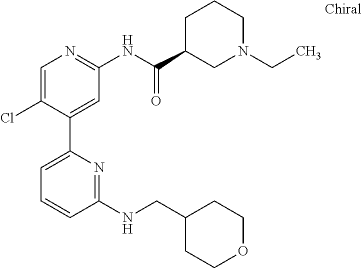 | 458.2 | 0.50 | (S)-1-Ethyl-piperidine- 3-carboxylic acid {5′- chloro-6-[(tetrahydro- pyran-4-ylmethyl)- amino]- [2,4′]bipyridinyl-2′-yl}- amide |
| 15 | 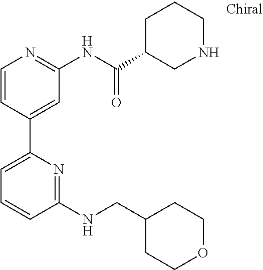 | 396.1 | 0.45 | (R)-Piperidine-3- carboxylic acid {6- [(tetrahydro-pyran-4- ylmethyl)-amino]- [2,4′]bipyridinyl-2′-yl}- amide |
| 16 | 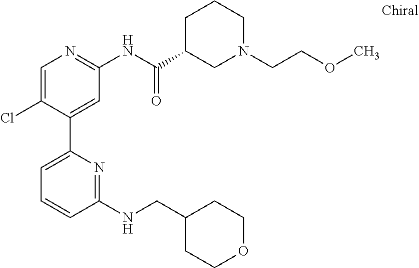 | 488.2 | 0.50 | (R)-1-(2-Methoxy- ethyl)-piperidine-3- carboxylic acid {5′- chloro-6-[(tetrahydro- pyran-4-ylmethyl)- amino]- [2,4′]bipyridinyl-2′-yl}- amide |
| 17 | 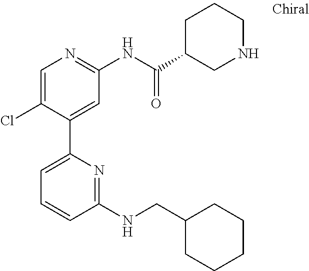 | 428.2 | 0.65 | (R)-Piperidine-3- carboxylic acid [5′- chloro-6- (cyclohexylmethyl- amino)- [2,4′]bipyridinyl-2′-yl]- amide |
| 18 | 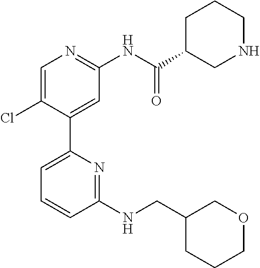 | 430.2 | 0.48 | (R)-Piperidine-3- carboxylic acid {5′- chloro-6-[(tetrahydro- pyran-3-ylmethyl)- amino]- [2,4′]bipyridinyl-2′-yl}- amide |
| 19 | 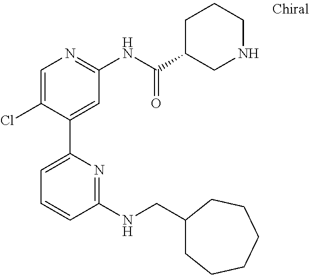 | 442.2 | 0.70 | (R)-Piperidine-3- carboxylic acid [5′- chloro-6- (cycloheptylmethyl- amino)- [2,4′]bipyridinyl-2′-yl]- amide |
| 20 | 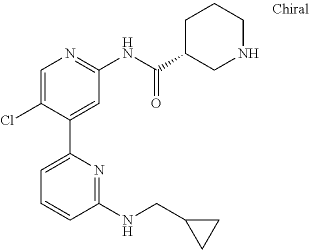 | 386.1 | 0.51 | (R)-Piperidine-3- carboxylic acid [5′- chloro-6- (cyclopropylmethyl- amino)- [2,4′]bipyridinyl-2′-yl]- amide |
| 21 | 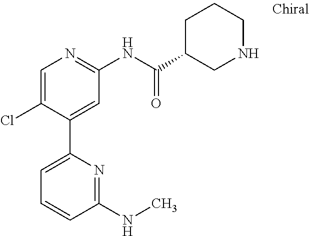 | 346 | 0.41 | (R)-Piperidine-3- carboxylic acid (5′- chloro-6-methylamino- [2,4′]bipyridinyl-2′-yl)- amide |
| 22 | 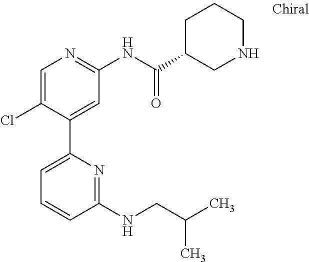 | 388.1 | 0.56 | (R)-Piperidine-3- carboxylic acid (5′- chloro-6- isobutylamino- [2,4′]bipyridinyl-2′-yl)- amide |
| 23 | 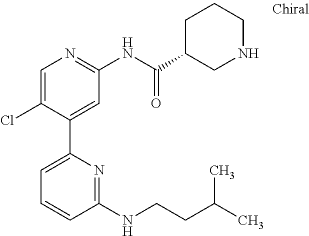 | 402.1 | 0.63 | (R)-Piperidine-3- carboxylic acid [5′- chloro-6-(3-methyl- butylamino)- [2,4′]bipyridinyl-2′-yl]- amide |
| 24 | 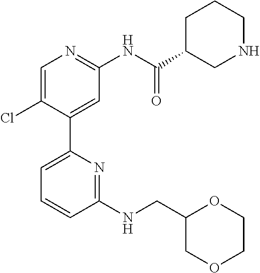 | 432.1 | 0.47 | (R)-Piperidine-3- carboxylic acid {5′- chloro-6-[([1,4]dioxan- 2-ylmethyl)-amino]- [2,4′]bipyridinyl-2′-yl}- amide |
| 25 | 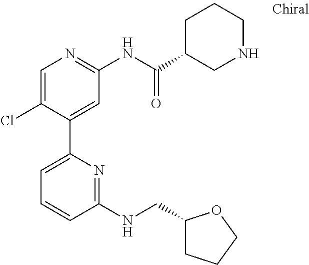 | 416.1 | 0.49 | (R)-Piperidine-3- carboxylic acid (5′- chloro-6-{[(R)-1- (tetrahydro-furan-2- yl)methyl]-amino}- [2,4′]bipyridinyl-2′-yl)- amide |
| 26 | 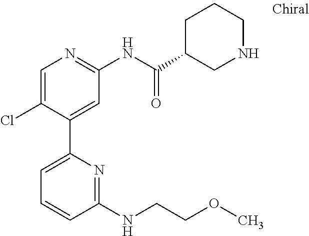 | 390.1 | 0.46 | (R)-Piperidine-3- carboxylic acid [5′- chloro-6-(2-methoxy- ethylamino)- [2,4′]bipyridinyl-2′-yl]- amide |
| 27 | 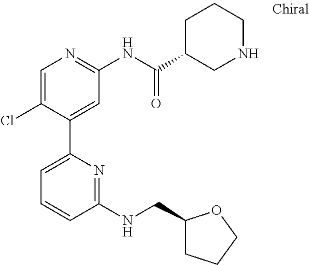 | 416.1 | 0.49 | (R)-Piperidine-3- carboxylic acid (5′- chloro-6-{[(S)-1- (tetrahydro-furan-2- yl)methyl]-amino}- [2,4′]bipyridinyl-2′-yl)- amide |
| 28 | 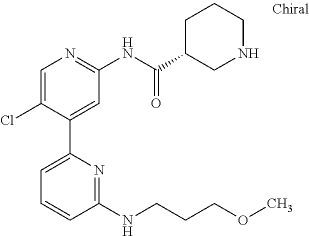 | 404.1 | 0.49 | (R)-Piperidine-3- carboxylic acid [5′- chloro-6-(3-methoxy- propylamino)- [2,4′]bipyridinyl-2′-yl]- amide |
| 29 | 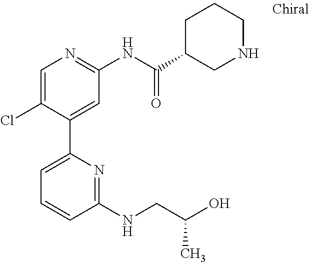 | 390.1 | 0.42 | (R)-Piperidine-3- carboxylic acid [5′- chloro-6-((R)-2- hydroxy-propylamino)- [2,4′]bipyridinyl-2′-yl]- amide |
| 30 | 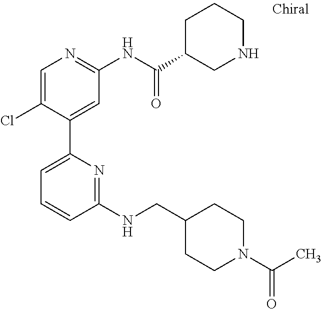 | 471.2 | 0.49 | (R)-Piperidine-3- carboxylic acid {6-[(1- acetyl-piperidin-4- ylmethyl)-amino]-5′- chloro- [2,4′]bipyridinyl-2′-yl}- amide |
| 31 | 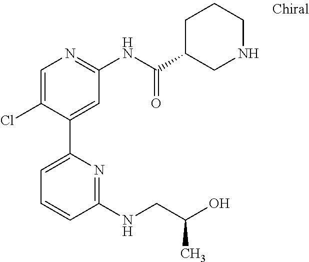 | 390.1 | 0.43 | (R)-Piperidine-3- carboxylic acid [5′- chloro-6-((S)-2- hydroxy-propylamino)- [2,4′]bipyridinyl-2′-yl]- amide |
| 32 | 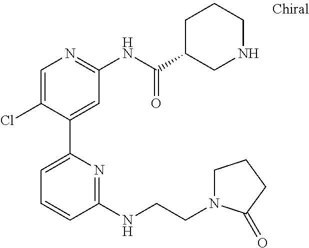 | 443.2 | 0.45 | (R)-Piperidine-3- carboxylic acid {5′- chloro-6-[2-(2-oxo- pyrrolidin-1-yl)- ethylamino]- [2,4′]bipyridinyl-2′-yl}- amide |
| 33 | 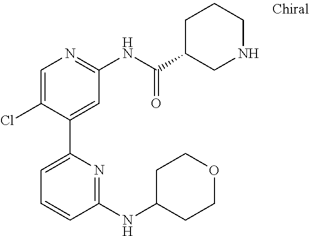 | 416.1 | 0.48 | (R)-Piperidine-3- carboxylic acid [5′- chloro-6-(tetrahydro- pyran-4-ylamino)- [2,4′]bipyridinyl-2′-yl]- amide |
| 34 | 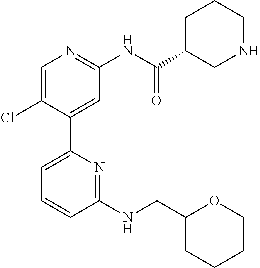 | 430.1 | 0.55 | (R)-Piperidine-3- carboxylic acid {5′- chloro-6-[(tetrahydro- pyran-2-ylmethyl)- amino]- [2,4′]bipyridinyl-2′-yl}- amide |
| 35 | 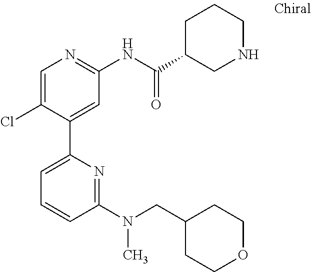 | 444.2 | 0.61 | (R)-Piperidine-3- carboxylic acid {5′- chloro-6-[methyl- (tetrahydro-pyran-4- ylmethyl)-amino]- [2,4′]bipyridinyl-2′-yl}- amide |
| 36 | 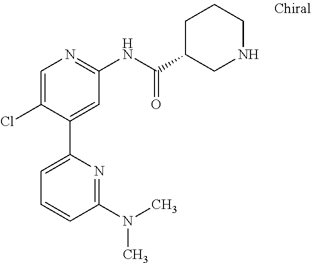 | 360.1 | 0.47 | (R)-Piperidine-3- carboxylic acid (5′- chloro-6- dimethylamino- [2,4′]bipyridinyl-2′-yl)- amide |
| 37 | 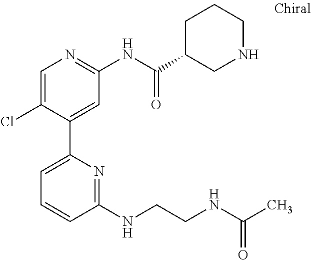 | 417.2 | 0.40 | (R)-Piperidine-3- carboxylic acid [6-(2- acetylamino- ethylamino)-5′-chloro- [2,4′]bipyridinyl-2′-yl]- amide |
| 38 | 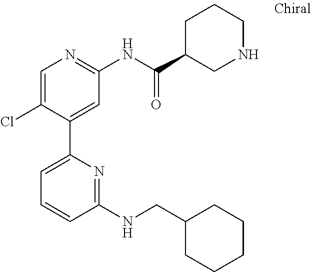 | 428.2 | 0.69 | (S)-Piperidine-3- carboxylic acid [5′- chloro-6- (cyclohexylmethyl- amino)- [2,4′]bipyridinyl-2′-yl]- amide |
| 39 | 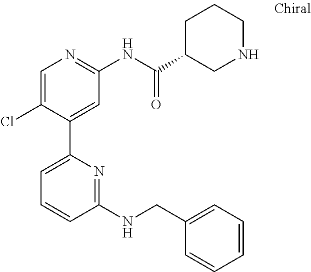 | 422.2 | 0.58 | (R)-Piperidine-3- carboxylic acid (6- benzylamino-5′-chloro- [2,4′]bipyridinyl-2′-yl)- amide |
| 40 | 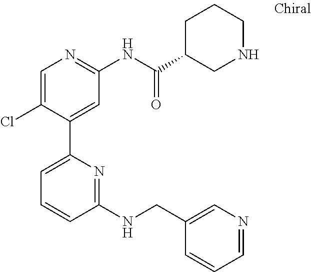 | 423.2 | 0.44 | (R)-Piperidine-3- carboxylic acid {5′- chloro-6-[(pyridin-3- ylmethyl)-amino]- [2,4′]bipyridinyl-2′-yl}- amide |
| 41 | 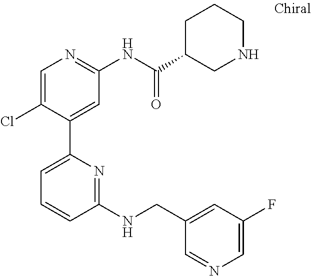 | 441.1 | 0.51 | (R)-Piperidine-3- carboxylic acid {5′- chloro-6-[(5-fluoro- pyridin-3-ylmethyl)- amino]- [2,4′]bipyridinyl-2′-yl}- amide |
| 42 | 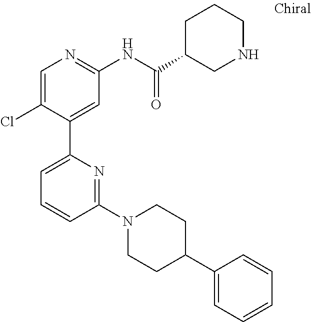 | 476.2 | 0.78 | (R)-Piperidine-3- carboxylic acid (5″- chloro-4-phenyl- 3,4,5,6-tetrahydro-2H- [1,2′;6′,4″]terpyridin-2″- yl)-amide |
| 43 | 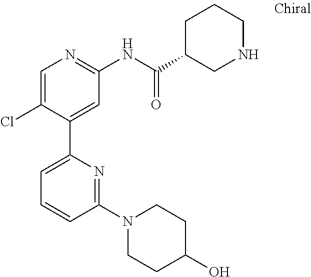 | 416.2 | 0.47 | (R)-Piperidine-3- carboxylic acid (5″- chloro-4-hydroxy- 3,4,5,6-tetrahydro-2H- [1,2′;6′,4″]terpyridin-2″- yl)-amide |
| 44 | 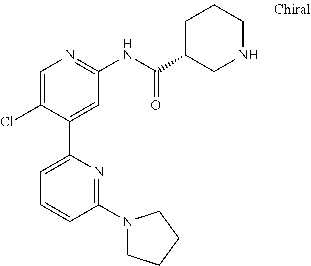 | 386.1 | 0.46 | (R)-Piperidine-3- carboxylic acid (5′- chloro-6-pyrrolidin-1- yl-[2,4′]bipyridinyl-2′- yl)-amide |
| 45 | 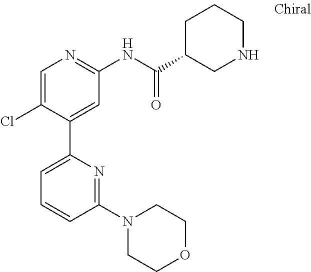 | 402.2 | 0.57 | (R)-Piperidine-3- carboxylic acid (5′- chloro-6-morpholin-4- yl-[2,4′]bipyridinyl-2′- yl)-amide |
| 46 | 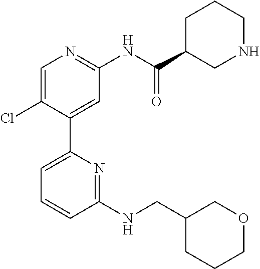 | 430.2 | 0.48 | (S)-Piperidine-3- carboxylic acid {5′- chloro-6-[(tetrahydro- pyran-3-ylmethyl)- amino]- [2,4′]bipyridinyl-2′-yl}- amide |
| 47 | 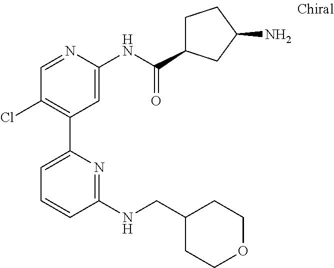 | 430.2 | 0.46 | (1S,3R)-3-Amino- cyclopentanecarboxylic acid {5′-chloro-6- [(tetrahydro-pyran-4- ylmethyl)-amino]- [2,4′]bipyridinyl-2′-yl}- amide |
| 48 | 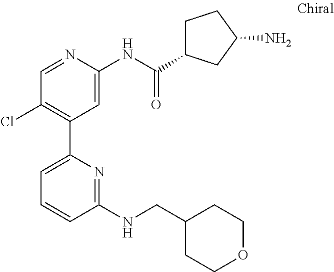 | 430.2 | 0.46 | (1R,3S)-3-Amino- cyclopentanecarboxylic acid {5′-chloro-6- [(tetrahydro-pyran-4- ylmethyl)-amino]- [2,4′]bipyridinyl-2′-yl}- amide |
| 49 | 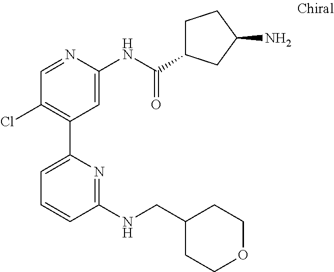 | 430.2 | 0.46 | (1R,3R)-3-Amino- cyclopentanecarboxylic acid {5′-chloro-6- [(tetrahydro-pyran-4- ylmethyl)-amino]- [2,4′]bipyridinyl-2′-yl}- amide |
| 50 | 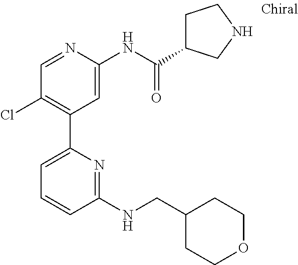 | 416.2 | 0.45 | (R)-Pyrrolidine-3- carboxylic acid {5′- chloro-6-[(tetrahydro- pyran-4-ylmethyl)- amino]- [2,4′]bipyridinyl-2′-yl}- amide |
| 51 | 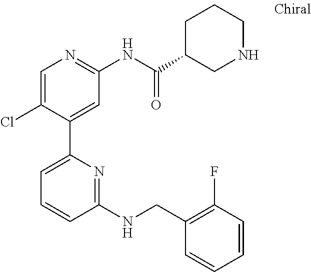 | 440.1 | 0.60 | (R)-Piperidine-3- carboxylic acid [5′- chloro-6-(2-fluoro- benzylamino)- [2,4′]bipyridinyl-2′-yl]- amide |
| 52 | 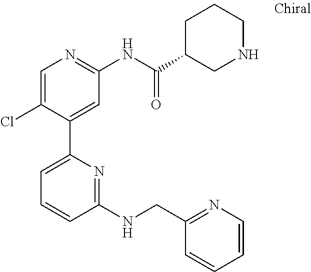 | 423.2 | 0.46 | (R)-Piperidine-3- carboxylic acid {5′- chloro-6-[(pyridin-2- ylmethyl)-amino]- [2,4′]bipyridinyl-2′-yl}- amide |
| 53 | 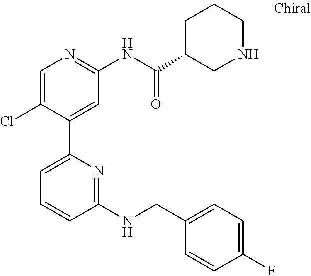 | 440.1 | 0.60 | (R)-Piperidine-3- carboxylic acid [5′- chloro-6-(4-fluoro- benzylamino)- [2,4′]bipyridinyl-2′-yl]- amide |
| 54 | 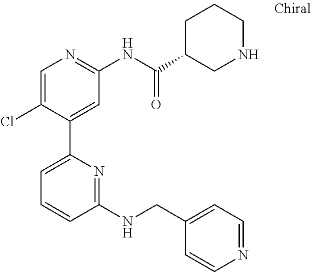 | 423.2 | 0.45 | (R)-Piperidine-3- carboxylic acid {5′- chloro-6-[(pyridin-4- ylmethyl)-amino]- [2,4′]bipyridinyl-2′-yl}- amide |
| 55 | 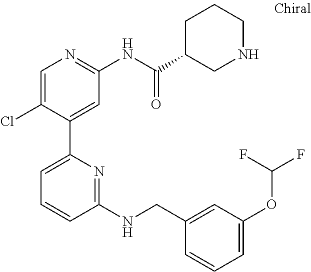 | 488.1 | 0.64 | (R)-Piperidine-3- carboxylic acid [5′- chloro-6-(3- difluoromethoxy- benzylamino)- [2,4′]bipyridinyl-2′-yl]- amide |
| 56 | 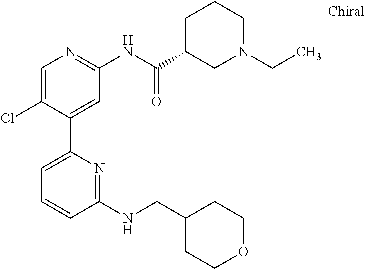 | 458.2 | 0.51 | (R)-1-Ethyl-piperidine- 3-carboxylic acid {5′- chloro-6-[(tetrahydro- pyran-4-ylmethyl)- amino]- [2,4′]bipyridinyl-2′-yl}- amide |
| 57 | 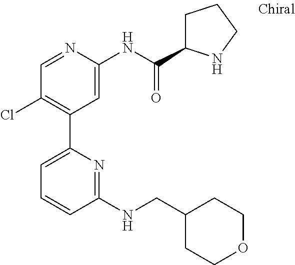 | 416.2 | 0.48 | (R)-Pyrrolidine-2- carboxylic acid {5′- chloro-6-[(tetrahydro- pyran-4-ylmethyl)- amino]- [2,4′]bipyridinyl-2′-yl}- amide |
| 58 | 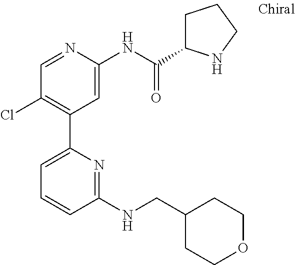 | 416.2 | 0.48 | (S)-Pyrrolidine-2- carboxylic acid {5′- chloro-6-[(tetrahydro- pyran-4-ylmethyl)- amino]- [2,4′]bipyridinyl-2′-yl}- amide |
| 59 | 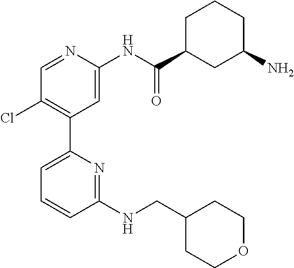 | 444.3 | 0.51 | (1S,3R)-/(1R,3S)-3- Amino- cyclohexanecarboxylic acid {5′-chloro-6- [(tetrahydro-pyran-4- ylmethyl)-amino]- [2,4′]bipyridinyl-2′-yl}- amide |
| 60 | 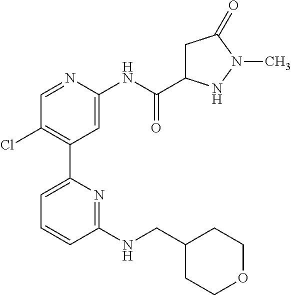 | 444.2 | 0.50 | 1-Methyl-5-oxo- pyrrolidine-3- carboxylic acid {5′- chloro-6-[(tetrahydro- pyran-4-ylmethyl)- amino]- [2,4′]bipyridinyl-2′-yl}- amide |
| 61 | 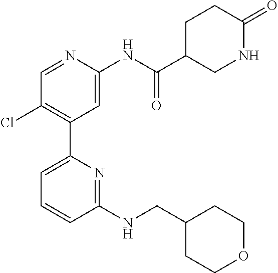 | 444.2 | 0.50 | 6-Oxo-piperidine-3- carboxylic acid {5′- chloro-6-[(tetrahydro- pyran-4-ylmethyl)- amino]- [2,4′]bipyridinyl-2′-yl}- amide |
| 62 | 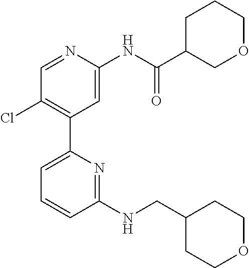 | 431.2 | 0.59 | Tetrahydro-pyran-3- carboxylic acid {5′- chloro-6-[(tetrahydro- pyran-4-ylmethyl)- amino]- [2,4′]bipyridinyl-2′-yl}- amide |
| 63 | 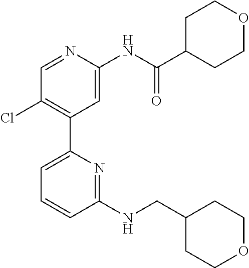 | 431.2 | 0.56 | Tetrahydro-pyran-4- carboxylic acid {5′- chloro-6-[(tetrahydro- pyran-4-ylmethyl)- amino]- [2,4′]bipyridinyl-2′-yl}- amide |
| 64 | 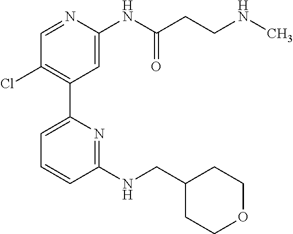 | 404.2 | 0.45 | N-{5′-Chloro-6- [(tetrahydro-pyran-4- ylmethyl)-amino]- [2,4′]bipyridinyl-2′-yl}- 3-methylamino- propionamide |
| 65 | 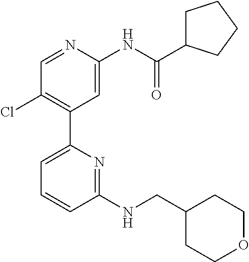 | 415.2 | 0.71 | Cyclopentanecarboxylic acid {5′-chloro-6- [(tetrahydro-pyran-4- ylmethyl)-amino]- [2,4′]bipyridinyl-2′-yl}- amide |
| 66 | 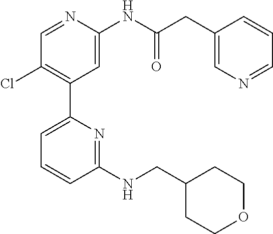 | 438.2 | 0.46 | N-{5′-Chloro-6- [(tetrahydro-pyran-4- ylmethyl)-amino]- [2,4′]bipyridinyl-2′-yl}- 2-pyridin-3-yl- acetamide |
| 67 | 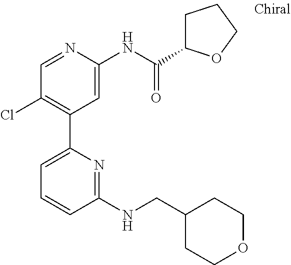 | 417.2 | 0.60 | (S)-Tetrahydro-furan- 2-carboxylic acid {5′- chloro-6-[(tetrahydro- pyran-4-ylmethyl)- amino]- [2,4′]bipyridinyl-2′-yl}- amide |
| 68 | 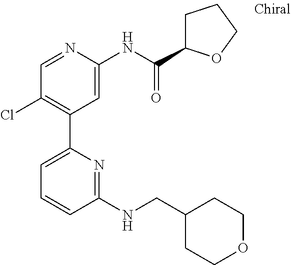 | 417.2 | 0.60 | (R)-Tetrahydro-furan- 2-carboxylic acid {5′- chloro-6-[(tetrahydro- pyran-4-ylmethyl)- amino]- [2,4′]bipyridinyl-2′-yl}- amide |
| 69 | 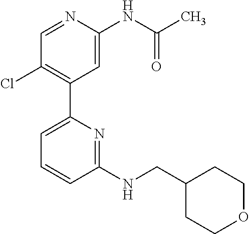 | 361.1 | 0.54 | N-{5′-Chloro-6- [(tetrahydro-pyran-4- ylmethyl)-amino]- [2,4′]bipyridinyl-2′-yl}- acetamide |
| 70 | 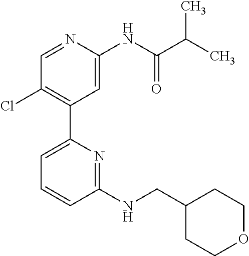 | 389.2 | 0.65 | N-{5′-Chloro-6- [(tetrahydro-pyran-4- ylmethyl)-amino]- [2,4′]bipyridinyl-2′-yl}- isobutyramide |
| 71 | 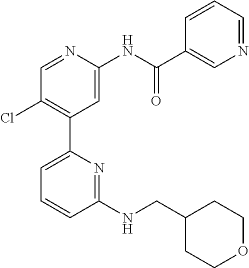 | 424.2 | 0.52 | N-{5′-Chloro-6- [(tetrahydro-pyran-4- ylmethyl)-amino]- [2,4′]bipyridinyl-2′-yl}- nicotinamide |
| 72 | 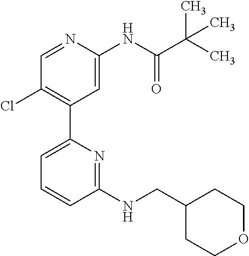 | 403.2 | 0.68 | N-{5′-Chloro-6- [(tetrahydro-pyran-4- ylmethyl)-amino]- [2,4′]bipyridinyl-2′-yl}- 2,2-dimethyl- propionamide |
| 73 | 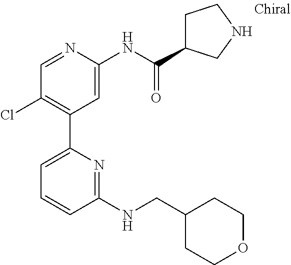 | 416.2 | 0.46 | (S)-Pyrrolidine-3- carboxylic acid {5′- chloro-6-[(tetrahydro- pyran-4-ylmethyl)- amino]- [2,4′]bipyridinyl-2′-yl}- amide |
| 74 | 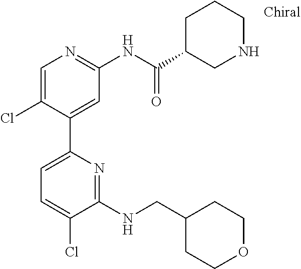 | 464.2 | 0.79 | (R)-Piperidine-3- carboxylic acid {5,5′- dichloro-6-[(tetrahydro- pyran-4-ylmethyl)- amino]- [2,4′]bipyridinyl-2′-yl}- amide |
| 75 | 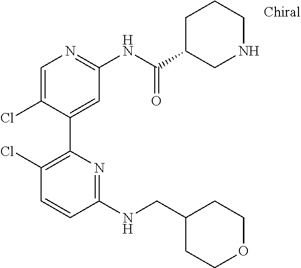 | 464.2 | 0.67 | (R)-Piperidine-3- carboxylic acid {3,5′- dichloro-6-[(tetrahydro- pyran-4-ylmethyl)- amino]- [2,4′]bipyridinyl-2′-yl}- amide |
| 76 | 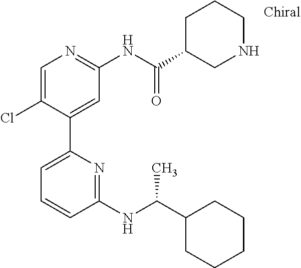 | 442.2 | 0.68 | (R)-Piperidine-3- carboxylic acid [5′- chloro-6-((R)-1- cyclohexyl- ethylamino)- [2,4′]bipyridinyl-2′-yl]- amide |
| 77 | 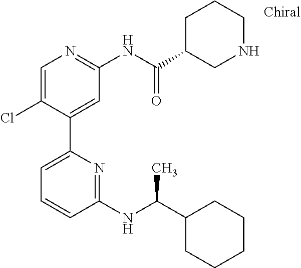 | 442.2 | 0.68 | (R)-Piperidine-3- carboxylic acid [5′- chloro-6-((S)-1- cyclohexyl- ethylamino)- [2,4′]bipyridinyl-2′-yl]- amide |
| 78 | 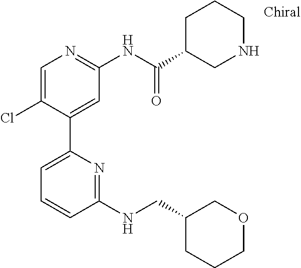 | 430.2 | 0.49 | (R)-Piperidine-3- carboxylic acid (5′- chloro-6-{[(R)-1- (tetrahydro-pyran-3- yl)methyl]-amino}- [2,4′]bipyridinyl-2′-yl)- amide |
| 79 | 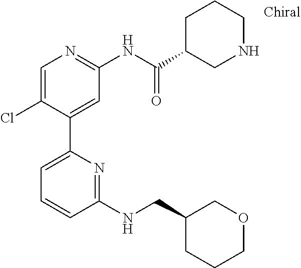 | 430.2 | 0.49 | (R)-Piperidine-3- carboxylic acid (5′- chloro-6-{[(S)-1- (tetrahydro-pyran-3- yl)methyl]-amino}- [2,4′]bipyridinyl-2′-yl)- amide |
| 80 | 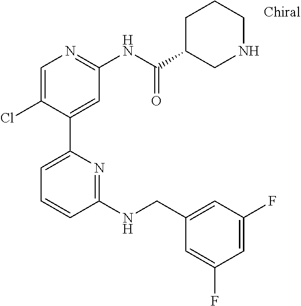 | 458.1 | 0.67 | (R)-Piperidine-3- carboxylic acid [5′- chloro-6-(3,5-difluoro- benzylamino)- [2,4′]bipyridinyl-2′-yl]- amide |
| 81 | 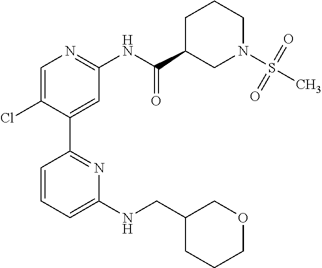 | 508.2 | 0.62 | (S)-1-Methanesulfonyl- piperidine-3-carboxylic acid {5′-chloro-6- [(tetrahydro-pyran-3- ylmethyl)-amino]- [2,4′]bipyridinyl-2′-yl}- amide |
| 82 | 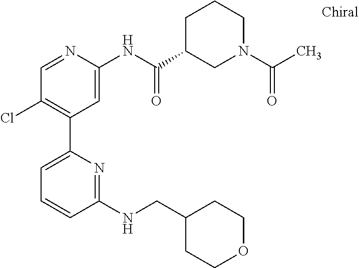 | 472.3 | 0.57 | (R)-1-Acetyl- piperidine-3-carboxylic acid {5′-chloro-6- [(tetrahydro-pyran-4- ylmethyl)-amino]- [2,4′]bipyridinyl-2′-yl}- amide |
| 83 | 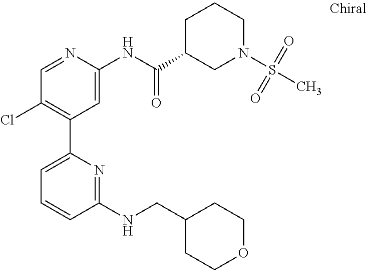 | 508.3 | 0.61 | (R)-1-Methanesulfonyl- piperidine-3-carboxylic acid {5′-chloro-6- [(tetrahydro-pyran-4- ylmethyl)-amino]- [2,4′]bipyridinyl-2′-yl}- amide |
| 84 | 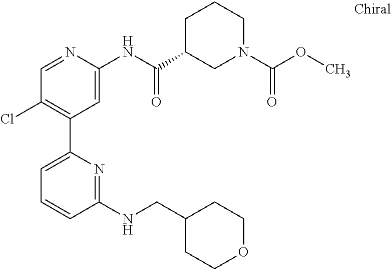 | 488.3 | 0.65 | (R)-3-{5′-Chloro-6- [(tetrahydro-pyran-4- ylmethyl)-amino]- [2,4′]bipyridinyl-2′- ylcarbamoyl}- piperidine-1-carboxylic acid methyl ester |
| 85 | 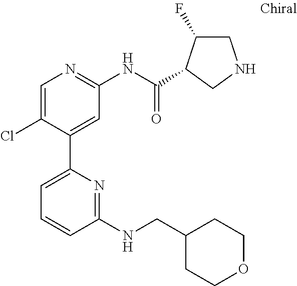 | 434 | 0.46 | (3R,4S)-4-Fluoro- pyrrolidine-3- carboxylic acid {5′- chloro-6-[(tetrahydro- pyran-4-ylmethyl)- amino]- [2,4′]bipyridinyl-2′-yl}- amide |
| 86 | 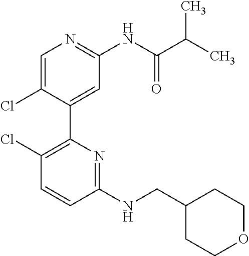 | 423.1 | 0.88 | N-{3,5′-Dichloro-6- [(tetrahydro-pyran-4- ylmethyl)-amino]- [2,4′]bipyridinyl-2′-yl}- isobutyramide |
| 87 | 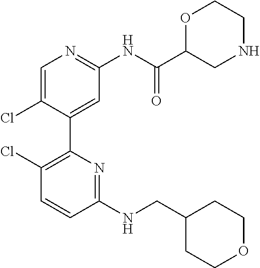 | 466.1 | 0.67 | Morpholine-2- carboxylic acid {3,5′- dichloro-6-[(tetrahydro- pyran-4-ylmethyl)- amino]- [2,4′]bipyridinyl-2′-yl}- amide |
| 88 | 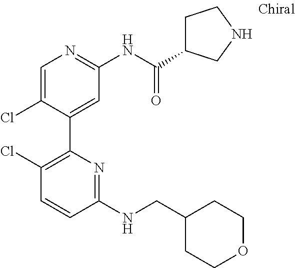 | 450.1 | 0.65 | (R)-Pyrrolidine-3- carboxylic acid {3,5′- dichloro-6-[(tetrahydro- pyran-4-ylmethyl)- amino]- [2,4′]bipyridinyl-2′-yl}- amide |
| 89 | 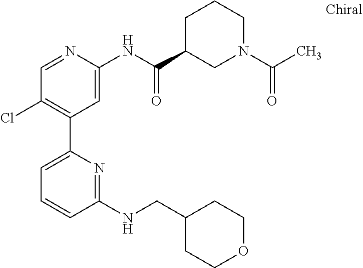 | 472.2 | 0.56 | (S)-1-Acetyl- piperidine-3-carboxylic acid {5′-chloro-6- [(tetrahydro-pyran-4- ylmethyl)-amino]- [2,4′]bipyridinyl-2′-yl}- amide |
| 90 | 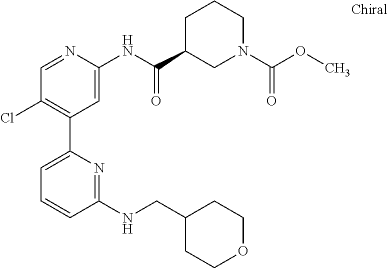 | 488.2 | 0.63 | (S)-3-{5′-Chloro-6- [(tetrahydro-pyran-4- ylmethyl)-amino]- [2,4′]bipyridinyl-2′- ylcarbamoyl}- piperidine-1-carboxylic acid methyl ester |
| 91 | 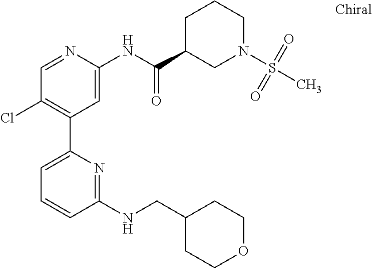 | 508.1 | 0.60 | (S)-1-Methanesulfonyl- piperidine-3-carboxylic acid {5′-chloro-6- [(tetrahydro-pyran-4- ylmethyl)-amino]- [2,4′]bipyridinyl-2′-yl}- amide |
| 92 | 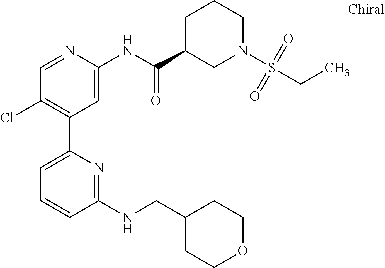 | 522.2 | 0.64 | (S)-1-Ethanesulfonyl- piperidine-3-carboxylic acid {5′-chloro-6- [(tetrahydro-pyran-4- ylmethyl)-amino]- [2,4′]bipyridinyl-2′-yl}- amide |
| 93 | 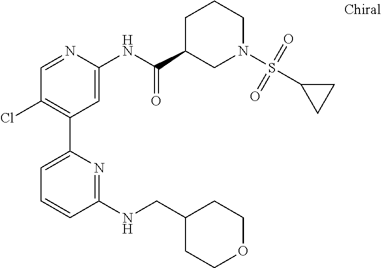 | 534.2 | 0.66 | (S)-1- Cyclopropanesulfonyl- piperidine-3-carboxylic acid {5′-chloro-6- [(tetrahydro-pyran-4- ylmethyl)-amino]- [2,4′]bipyridinyl-2′-yl}- amide |
| 94 | 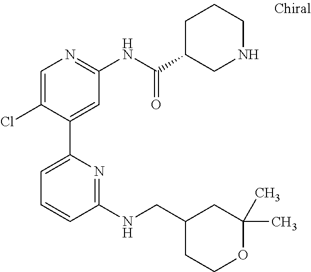 | 458.2 | 0.53 | (R)-Piperidine-3- carboxylic acid {5′- chloro-6-[(2,2- dimethyl-tetrahydro- pyran-4-ylmethyl)- amino]- [2,4′]bipyridinyl-2′-yl}- amide |
| 95 | 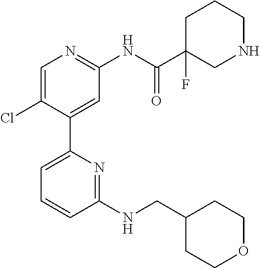 | 448.2 | 0.47 | 3-Fluoro-piperidine-3- carboxylic acid {5′- chloro-6-[(tetrahydro- pyran-4-ylmethyl)- amino]- [2,4′]bipyridinyl-2′-yl}- amide |
| 96 | 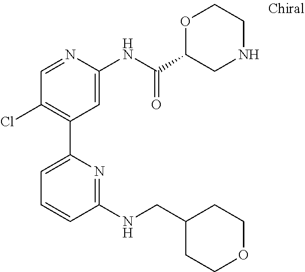 | 432.2 | 0.46 | (R)-Morpholine-2- carboxylic acid {5′- chloro-6-[(tetrahydro- pyran-4-ylmethyl)- amino]- [2,4′]bipyridinyl-2′-yl}- amide |
| 97 | 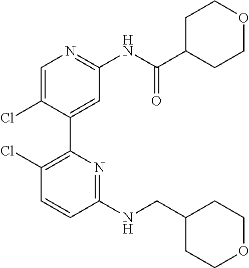 | 465.2 | 0.82 | Tetrahydro-pyran-4- carboxylic acid {3,5′- dichloro-6-[(tetrahydro- pyran-4-ylmethyl)- amino]- [2,4′]bipyridinyl-2′-yl}- amide |
| 98 | 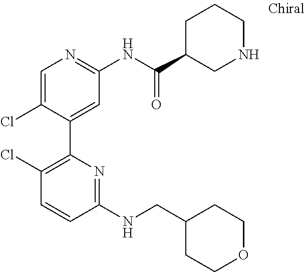 | 464.2 | 0.69 | (S)-Piperidine-3- carboxylic acid {3,5′- dichloro-6-[(tetrahydro- pyran-4-ylmethyl)- amino]- [2,4′]bipyridinyl-2′-yl}- amide |
| 99 | 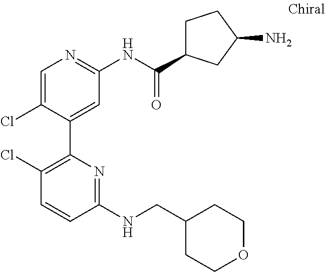 | 464.2 | 0.67 | (1S,3R)-3-Amino- cyclopentanecarboxylic acid {3,5′-dichloro-6- [(tetrahydro-pyran-4- ylmethyl)-amino]- [2,4′]bipyridinyl-2′-yl}- amide |
| 100 | 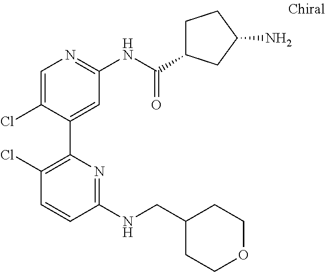 | 464.1 | 0.67 | (1R,3S)-3-Amino- cyclopentanecarboxylic acid {3,5′-dichloro-6- [(tetrahydro-pyran-4- ylmethyl)-amino]- [2,4′]bipyridinyl-2′-yl}- amide |
| 101 | 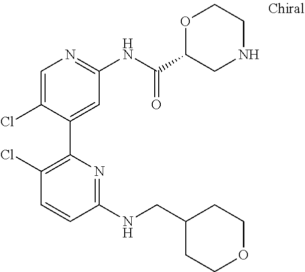 | 466.1 | 0.69 | (R)-Morpholine-2- carboxylic acid {3,5′- dichloro-6-[(tetrahydro- pyran-4-ylmethyl)- amino]- [2,4′]bipyridinyl-2′-yl}- amide |
| 102 | 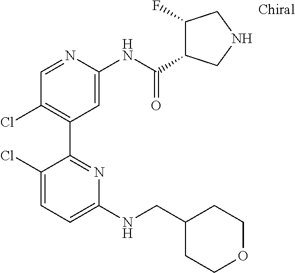 | 488.1 | 0.74 | (3R,4S)-4-Fluoro- pyrrolidine-3- carboxylic acid {3,5′- dichloro-6-[(tetrahydro- pyran-4-ylmethyl)- amino]- [2,4′]bipyridinyl-2′-yl}- amide |
| 103 | 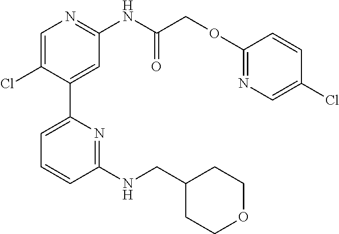 | 468.1 | 0.65 | 2-(5-Chloro-pyridin-2- yloxy)-N-{5′-chloro-6- [(tetrahydro-pyran-4- ylmethyl)-amino]- [2,4′]bipyridinyl-2′-yl}- acetamide |
| 104 | 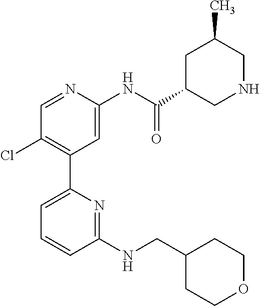 | 444.2 | 0.49 | (3R,5R)-/(3S,5S)-5- Methyl-piperidine-3- carboxylic acid {5′- chloro-6-[(tetrahydro- pyran-4-ylmethyl)- amino]- [2,4′]bipyridinyl-2′-yl}- amide |
| 105 | 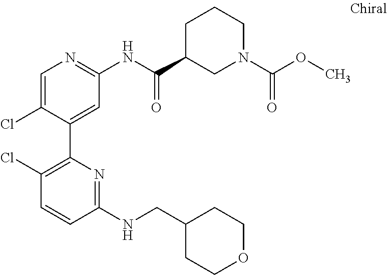 | 522.1 | 0.87 | (S)-3-{3,5′-Dichloro-6- [(tetrahydro-pyran-4- ylmethyl)-amino]- [2,4′]bipyridinyl-2′- ylcarbamoyl}- piperidine-1-carboxylic acid methyl ester |
| 106 | 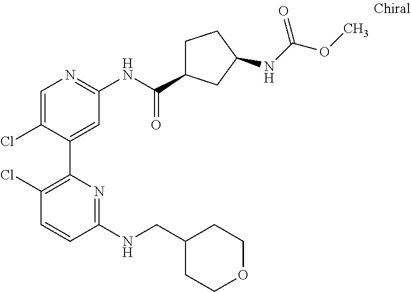 | 522.1 | 0.84 | ((lR,3S)-3-{3,5′- Dichloro-6- [(tetrahydro-pyran-4- ylmethyl)-amino]- [2,4′]bipyridinyl-2′- ylcarbamoyl}- cyclopentyl)-carbamic acid methyl ester |
| 107 | 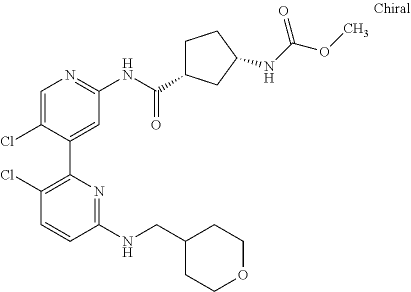 | 522.1 | 0.84 | ((lS,3R)-3-{3,5′- Dichloro-6- [(tetrahydro-pyran-4- ylmethyl)-amino]- [2,4′]bipyridinyl-2′- ylcarbamoyl}- cyclopentyl)-carbamic acid methyl ester |
| 108 | 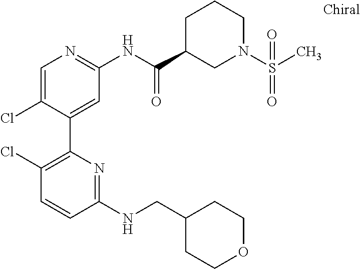 | 542.1 | 0.83 | (S)-1-Methanesulfonyl- piperidine-3-carboxylic acid {3,5′-dichloro-6- [(tetrahydro-pyran-4- ylmethyl)-amino]- [2,4′]bipyridinyl-2′-yl}- amide |
| 109 | 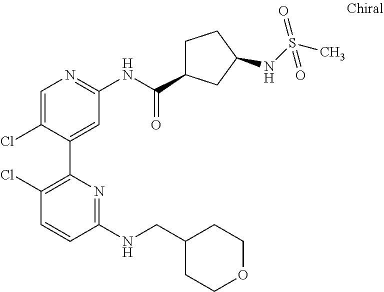 | 542.1 | 0.79 | (1S,3R)-3- Methanesulfonylamino- cyclopentanecarboxylic acid {3,5′-dichloro-6- [(tetrahydro-pyran-4- ylmethyl)-amino]- [2,4′]bipyridinyl-2′-yl}- amide |
| 110 | 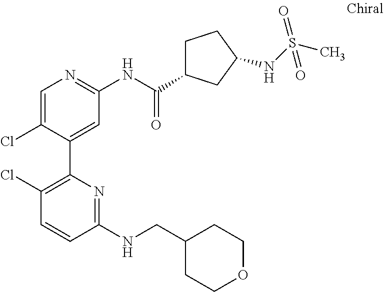 | 542.1 | 0.79 | (1R,3S)-3- Methanesulfonylamino- cyclopentanecarboxylic acid {3,5′-dichloro-6- [(tetrahydro-pyran-4- ylmethyl)-amino]- [2,4′]bipyridinyl-2′-yl}- amide |
| 111 | 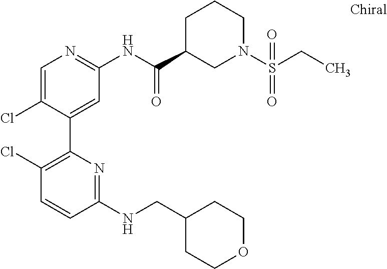 | 556.2 | 0.87 | (S)-1-Ethanesulfonyl- piperidine-3-carboxylic acid {3,5′-dichloro-6- [(tetrahydro-pyran-4- ylmethyl)-amino]- [2,4′]bipyridinyl-2′-yl}- amide |
| 112 | 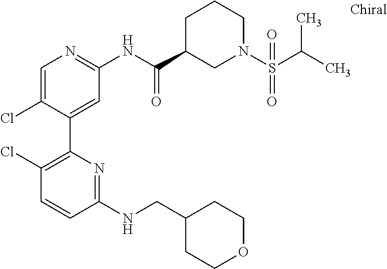 | 570.1/572.1 | 0.91 | (S)-1-(Propane-2- sulfonyl)-piperidine-3- carboxylic acid {3,5′- dichloro-6-[(tetrahydro- pyran-4-ylmethyl)- amino]- [2,4′]bipyridinyl-2′-yl}- amide |
| 113 | 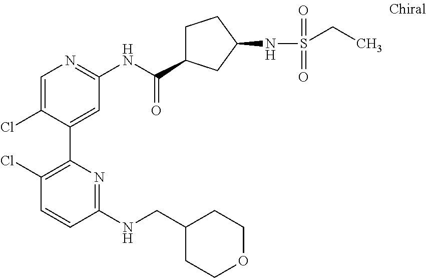 | 556.2 | 0.83 | (1S,3R)-3- Ethanesulfonylamino- cyclopentanecarboxylic acid {3,5′-dichloro-6- [(tetrahydro-pyran-4- ylmethyl)-amino]- [2,4′]bipyridinyl-2′-yl}- amide |
| 114 | 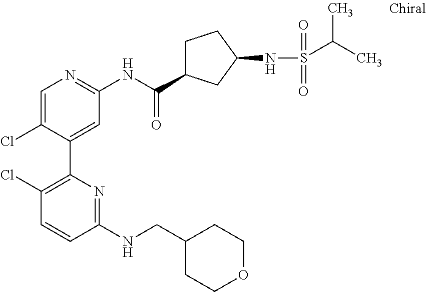 | 570.1 | 0.87 | (1S,3R)-3-(Propane-2- sulfonylamino)- cyclopentanecarboxylic acid {3,5′-dichloro-6- [(tetrahydro-pyran-4- ylmethyl)-amino]- [2,4′]bipyridinyl-2′-yl}- amide |
| 115 | 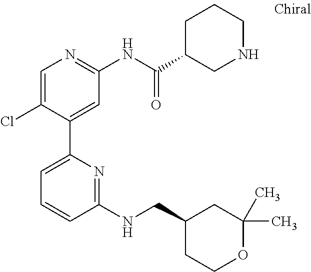 | 458.2 | 0.52 | (R)-Piperidine-3- carboxylic acid {5′- chloro-6-[((R)-2,2- dimethyl-tetrahydro- pyran-4-ylmethyl)- amino]- [2,4′]bipyridinyl-2′-yl}- amide |
| 116 | 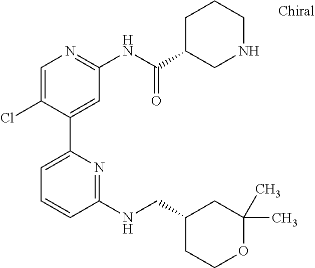 | 458.2 | 0.52 | (R)-Piperidine-3- carboxylic acid {5′- chloro-6-[((S)-2,2- dimethyl-tetrahydro- pyran-4-ylmethyl)- amino]- [2,4′]bipyridinyl-2′-yl}- amide |
| 117 | 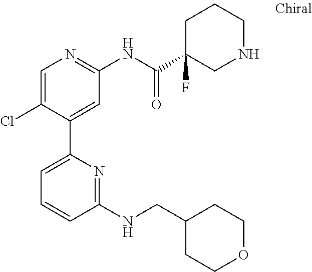 | n.a. | n.a. | (S)-3-Fluoro- piperidine-3-carboxylic acid {5′-chloro-6- [(tetrahydro-pyran-4- ylmethyl)-amino]- [2,4′]bipyridinyl-2′-yl}- amide |
| 118 | 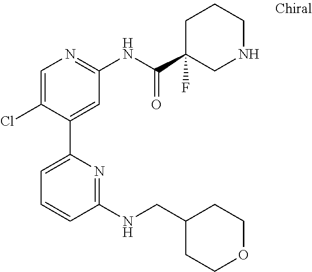 | n.a. | n.a. | (R)-3-Fluoro- piperidine-3-carboxylic acid {5′-chloro-6- [(tetrahydro-pyran-4- ylmethyl)-amino]- [2,4′]bipyridinyl-2′-yl}- amide |
| 119 | 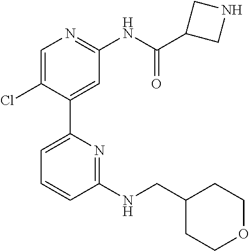 | 402.2 | 0.41 | Azetidine-3-carboxylic acid {5′-chloro-6- [(tetrahydro-pyran-4- ylmethyl)-amino]- [2,4′]bipyridinyl-2′-yl}- amide |
| 120 | 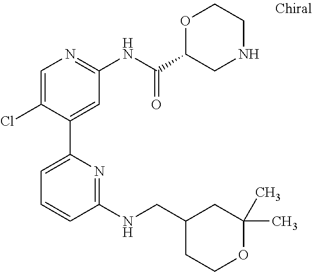 | 460.1 | 0.52 | (R)-Morpholine-2- carboxylic acid {5′- chloro-6-[(2,2- dimethyl-tetrahydro- pyran-4-ylmethyl)- amino]- [2,4′]bipyridinyl-2′-yl}- amide |
| 121 | 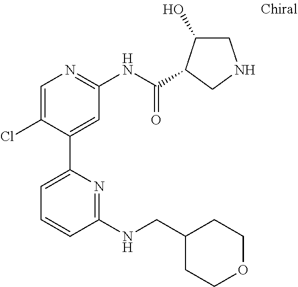 | 432.1 | 0.44 | (3S,4S)-4-Hydroxy- pyrrolidine-3- carboxylic acid {5′- chloro-6-[(tetrahydro- pyran-4-ylmethyl)- amino]- [2,4′]bipyridinyl-2′-yl}- amide |
| 122 | 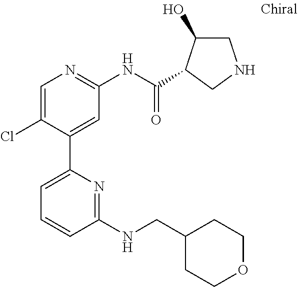 | 432.1 | 0.44 | (3S,4R)-4-Hydroxy- pyrrolidine-3- carboxylic acid {5′- chloro-6-[(tetrahydro- pyran-4-ylmethyl)- amino]- [2,4′]bipyridinyl-2′-yl}- amide |
| 123 | 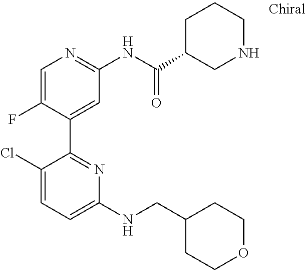 | 448.3 | 0.65 | (R)-Piperidine-3- carboxylic acid {3- chloro-5′-fluoro-6- [(tetrahydro-pyran-4- ylmethyl)-amino]- [2,4′]bipyridinyl-2′-yl}- amide |
| 124 | 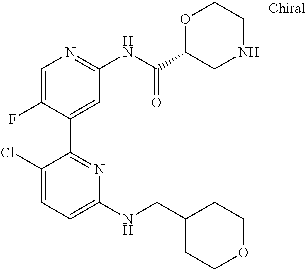 | 450.2 | 0.63 | (R)-Morpholine-2- carboxylic acid {3- chloro-5′-fluoro-6- [(tetrahydro-pyran-4- ylmethyl)-amino]- [2,4′]bipyridinyl-2′-yl}- amide |
| 125 | 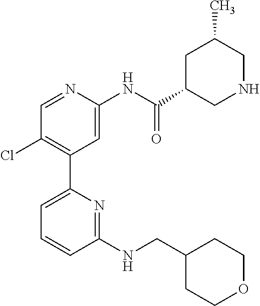 | 444.2 | 0.5 | (3R,5S)-/(3S,5R)-5- Methyl-piperidine-3- carboxylic acid {5′- chloro-6-[(tetrahydro- pyran-4-ylmethyl)- amino]- [2,4′]bipyridinyl-2′-yl}- amide |
| 126 | 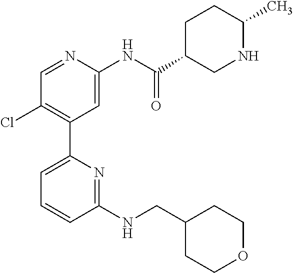 | 444.1 | 0.48 | (3R,6S)-/(3S,6R)-6- Methyl-piperidine-3- carboxylic acid {5′- chloro-6-[(tetrahydro- pyran-4-ylmethyl)- amino]- [2,4′]bipyridinyl-2′-yl}- amide |
| 127 | 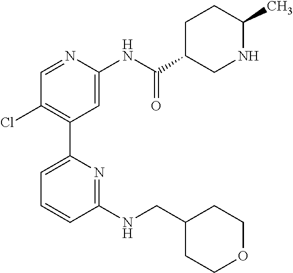 | 444.2 | 0.49 | (3R,6R)-/(3S,6S)-6- Methyl-piperidine-3- carboxylic acid {5′- chloro-6-[(tetrahydro- pyran-4-ylmethyl)- amino]- [2,4′]bipyridinyl-2′-yl}- amide |
| 128 | 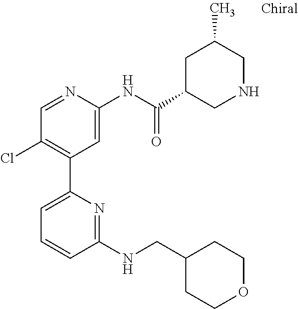 | 444.2 | 0.47 | (3R,5S)-5-Methyl- piperidine-3-carboxylic acid {5′-chloro-6- [(tetrahydro-pyran-4- ylmethyl)-amino]- [2,4′]bipyridinyl-2′-yl}- amide |
| 129 | 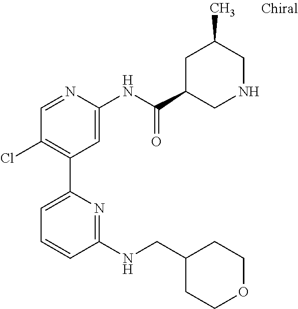 | 444.2 | 0.48 | (3S,5R)-5-Methyl- piperidine-3-carboxylic acid {5′-chloro-6- [(tetrahydro-pyran-4- ylmethyl)-amino]- [2,4′]bipyridinyl-2′-yl}- amide |
| 130 | 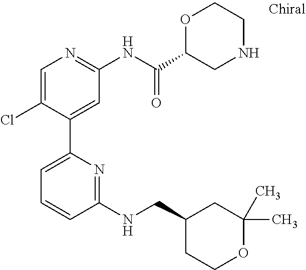 | n.a. | n.a. | (R)-Morpholine-2- carboxylic acid {5′- chloro-6-[((R)-2,2- dimethyl-tetrahydro- pyran-4-ylmethyl)- amino]- [2,4′]bipyridinyl-2′-yl}- amide |
| 131 | 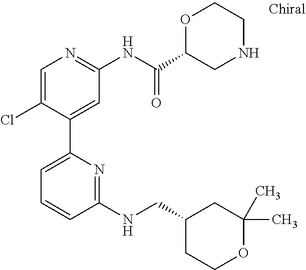 | n.a. | n.a. | (R)-Morpholine-2- carboxylic acid {5′- chloro-6-[((S)-2,2- dimethyl-tetrahydro- pyran-4-ylmethyl)- amino]- [2,4′]bipyridinyl-2′-yl}- amide |
| 132 | 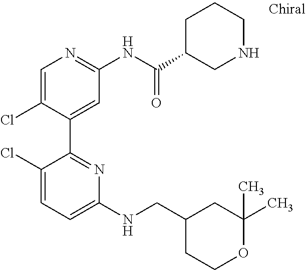 | 492.2/494.0 | 0.74 | (R)-Piperidine-3- carboxylic acid {3,5′- dichloro-6-[(2,2- dimethyl-tetrahydro- pyran-4-ylmethyl)- amino]- [2,4′]bipyridinyl-2′-yl}- amide |
| 133 | 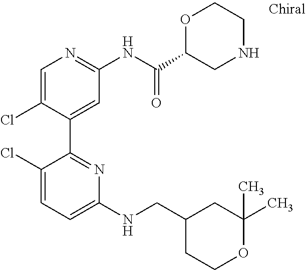 | 494.1/496.0 | 0.73 | (R)-Morpholine-2- carboxylic acid {3,5′- dichloro-6-[(2,2- dimethyl-tetrahydro- pyran-4-ylmethyl)- amino]- [2,4′]bipyridinyl-2′-yl}- amide |
| 134 | 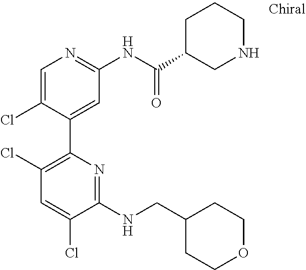 | 498.0/500.0 | 0.81 | (R)-Piperidine-3- carboxylic acid {3,5,5′- trichloro-6-[(tetrahydro- pyran-4-ylmethyl)- amino]- [2,4′]bipyridinyl-2′-yl}- amide |
| 135 | 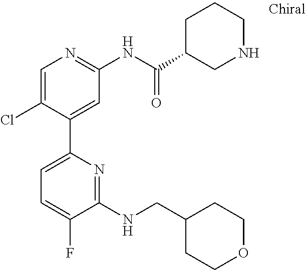 | 448.2 | 0.71 | (R)-Piperidine-3- carboxylic acid {5′- chloro-5-fluoro-6- [(tetrahydro-pyran-4- ylmethyl)-amino]- [2,4′]bipyridinyl-2′-yl}- amide |
| 136 | 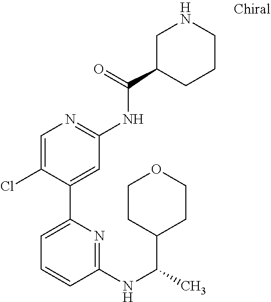 | 444.1 | 0.5 | (R)-Piperidine-3- carboxylic acid {5′- chloro-6-[(S)-1- (tetrahydro-pyran-4-yl)- ethylamino]- [2,4′]bipyridinyl-2′-yl}- amide |
| 137 | 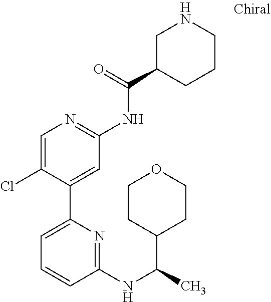 | 444.1 | 0.5 | (R)-Piperidine-3- carboxylic acid {5′- chloro-6-[(R)-1- (tetrahydro-pyran-4-yl)- ethylamino]- [2,4′]bipyridinyl-2′-yl}- amide |
| 138 | 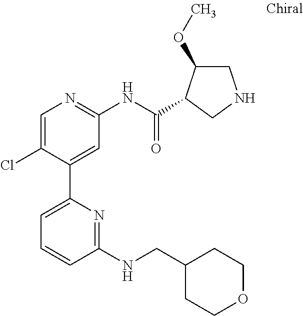 | 446.1 | 0.46 | (3S,4R)-4-Methoxy- pyrrolidine-3- carboxylic acid {5′- chloro-6-[(tetrahydro- pyran-4-ylmethyl)- amino]- [2,4′]bipyridinyl-2′-yl}- amide |
| 139 | 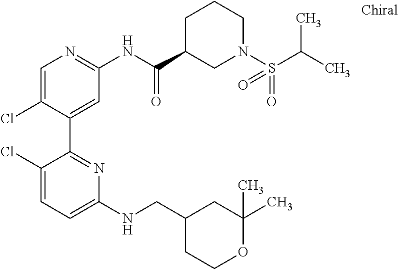 | 598.1/600.1 | 0.98 | (S)-1-(Propane-2- sulfonyl)-piperidine-3- carboxylic acid {3,5′- dichloro-6-[(2,2- dimethyl-tetrahydro- pyran-4-ylmethyl)- amino]- [2,4′]bipyridinyl-2′-yl}- amide |
| 140 | 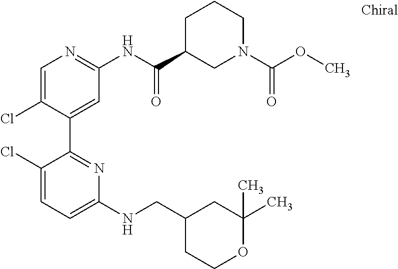 | 550.1/552.1 | 0.94 | (S)-3-{3,5′-Dichloro-6- [(2,2-dimethyl- tetrahydro-pyran-4- ylmethyl)-amino]- [2,4′]bipyridinyl-2′- ylcarbamoyl}- piperidine-1-carboxylic acid methyl ester |
| 141 | 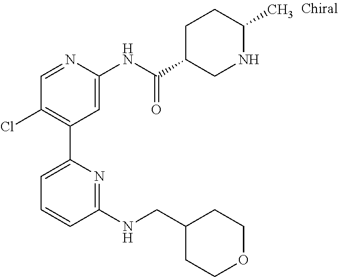 | 444.2 | 0.47 | (3R,6S)-6-Methyl- piperidine-3-carboxylic acid {5′-chloro-6- [(tetrahydro-pyran-4- ylmethyl)-amino]- [2,4′]bipyridinyl-2′-yl}- amide |
| 142 | 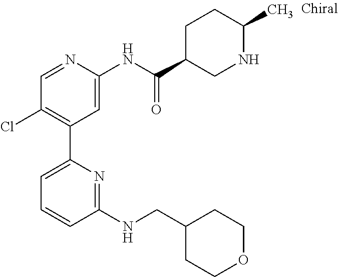 | 444.2 | 0.48 | (3S,6R)-6-Methyl- piperidine-3-carboxylic acid {5′-chloro-6- [(tetrahydro-pyran-4- ylmethyl)-amino]- [2,4′]bipyridinyl-2′-yl}- amide |
| 143 | 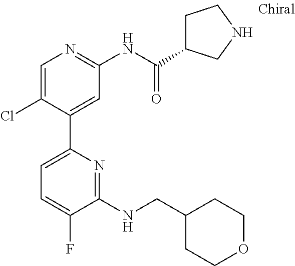 | 434.2 | 0.69 | (R)-Pyrrolidine-3- carboxylic acid {5′- chloro-5-fluoro-6- [(tetrahydro-pyran-4- ylmethyl)-amino]- [2,4′]bipyridinyl-2′-yl}- amide |
| 144 | 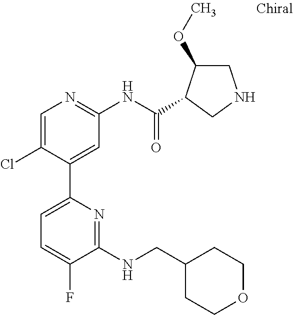 | 464.2 | 0.7 | (3S,4R)-4-Methoxy- pyrrolidine-3- carboxylic acid {5′- chloro-5-fluoro-6- [(tetrahydro-pyran-4- ylmethyl)-amino]- [2,4′]bipyridinyl-2′-yl}- amide |
| 145 | 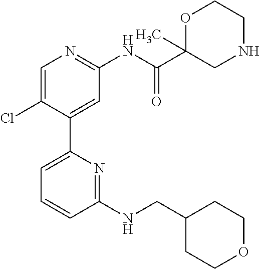 | 446.2 | 0.48 | 2-Methyl-morpholine- 2-carboxylic acid {5′- chloro-6-[(tetrahydro- pyran-4-ylmethyl)- amino]- [2,4′]bipyridinyl-2′-yl}- amide |
| 146 | 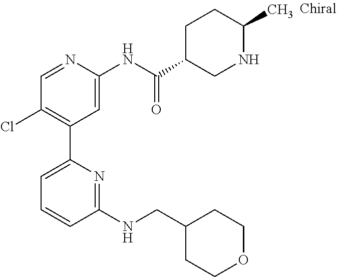 | 444.2 | 0.47 | (3R,6R)-6-Methyl- piperidine-3-carboxylic acid {5′-chloro-6- [(tetrahydro-pyran-4- ylmethyl)-amino]- [2,4′]bipyridinyl-2′-yl}- amide |
| 147 | 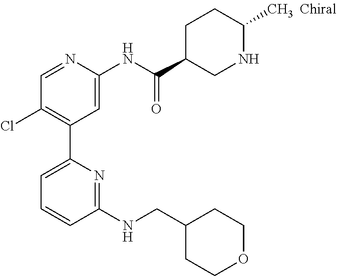 | 444.2 | 0.48 | (3S,6S)-6-Methyl- piperidine-3-carboxylic acid {5′-chloro-6- [(tetrahydro-pyran-4- ylmethyl)-amino]- [2,4′]bipyridinyl-2′-yl}- amide |
| 148 | 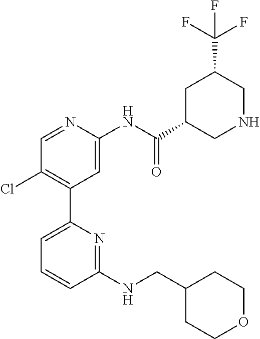 | 498.3 | 0.56 | (3R,5S)-/(3S,5R)-5- Trifluoromethyl- piperidine-3-carboxylic acid {5′-chloro-6- [(tetrahydro-pyran-4- ylmethyl)-amino]- [2,4′]bipyridinyl-2′-yl}- amide |
| 149 | 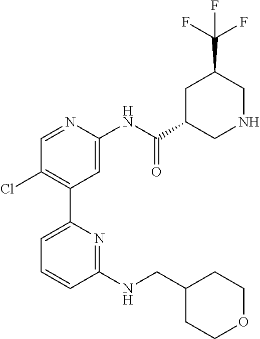 | 498.3 | 0.55 | (3R,5R)-/(3S,5S)-5- Trifluoromethyl- piperidine-3-carboxylic acid {5′-chloro-6- [(tetrahydro-pyran-4- ylmethyl)-amino]- [2,4′]bipyridinyl-2′-yl}- amide |
| 150 | 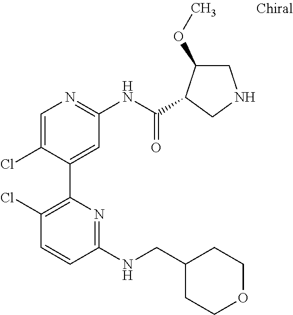 | 480.3 | 0.66 | (3S,4R)-4-Methoxy- pyrrolidine-3- carboxylic acid {3,5′- dichloro-6-[(tetrahydro- pyran-4-ylmethyl)- amino]- [2,4′]bipyridinyl-2′-yl}- amide |
| 151 | 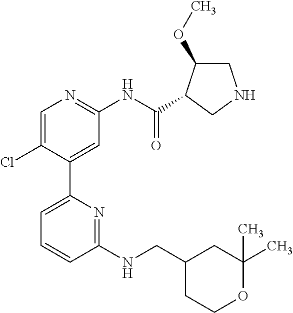 | 474.2 | 0.52 | (3S,4R)-4-Methoxy- pyrrolidine-3- carboxylic acid {5′- chloro-6-[(2,2- dimethyl-tetrahydro- pyran-4-ylmethyl)- amino]- [2,4′]bipyridinyl-2′-yl}- amide |
| 152 | 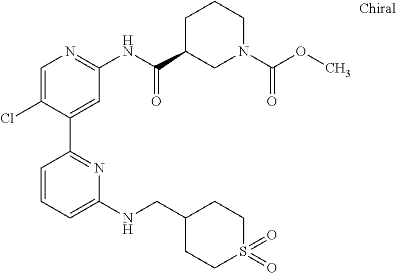 | 536.1 | 0.6 | (S)-3-{5′-Chloro-6- [(1′,1′-dioxo- hexahydro-1-thiopyran- 4-ylmethyl)-amino]- [2,4′]bipyridinyl-2′- ylcarbamoyl}- piperidine-1-carboxylic acid methyl ester |
| 153 | 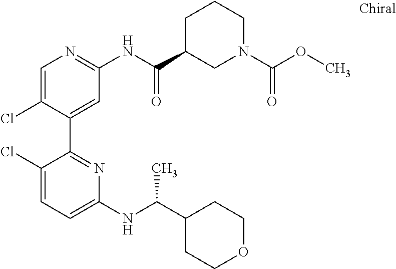 | 536.1 | 0.9 | (S)-3-{3,5′-Dichloro-6- [(R)-1-(tetrahydro- pyran-4-yl)- ethylamino]- [2,4′]bipyridinyl-2′- ylcarbamoyl}- piperidine-1-carboxylic acid methyl ester |
| 154 | 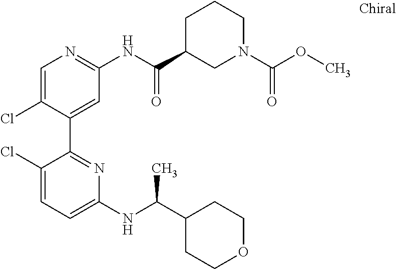 | 536.1 | 0.9 | (S)-3-{3,5′-Dichloro-6- [(S)-1-(tetrahydro- pyran-4-yl)- ethylamino]- [2,4′]bipyridinyl-2′- ylcarbamoyl}- piperidine-1-carboxylic acid methyl ester |
| 155 | 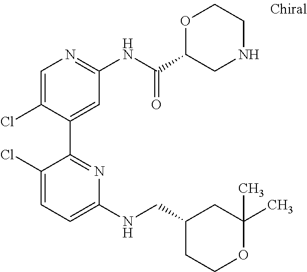 | 494.1/495.9 | 0.72 | (R)-Morpholine-2- carboxylic acid {3,5′- dichloro-6-[((S)-2,2- dimethyl-tetrahydro- pyran-4-ylmethyl)- amino]- [2,4′]bipyridinyl-2′-yl}- amide |
| 156 | 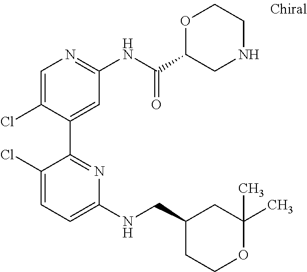 | 598.1/600.1 | 0.97 | (R)-Morpholine-2- carboxylic acid {3,5′- dichloro-6-[((R)-2,2- dimethyl-tetrahydro pyran-4-ylmethyl)- amino]- [2,4′]bipyridinyl-2′-yl}- amide |
| 157 | 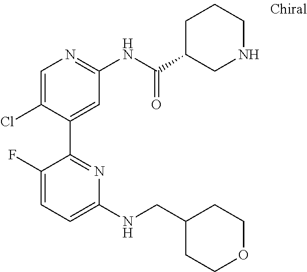 | 448.3 | 0.65 | (R)-Piperidine-3- carboxylic acid {5′- chloro-3-fluoro-6- [(tetrahydro-pyran-4- ylmethyl)-amino]- [2,4′]bipyridinyl-2′-yl}- amide |
| 158 | 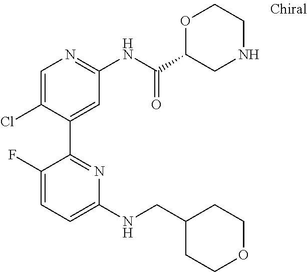 | 450.2 | 0.65 | (R)-Morpholine-2- carboxylic acid {5′- chloro-3-fluoro-6- [(tetrahydro-pyran-4- ylmethyl)-amino]- [2,4′]bipyridinyl-2′-yl}- amide |
| 159 | 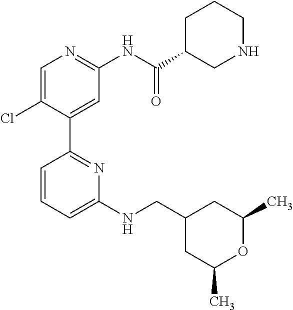 | 458.2 | 0.55 | (R)-Piperidine-3- carboxylic acid {5′- chloro-6-[((2R,6S)-2,6- dimethyl-tetrahydro- pyran-4-ylmethyl)- amino]- [2,4′]bipyridinyl-2′-yl}- amide |
| 160 | 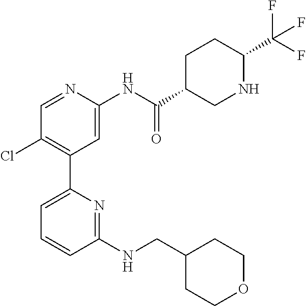 | 498.1 | 0.53 | (3R,6R)-/(3S,6S)-6- Trifluoromethyl- piperidine-3-carboxylic acid {5′-chloro-6- [(tetrahydro-pyran-4- ylmethyl)-amino]- [2,4′]bipyridinyl-2′-yl}- amide |
| 161 | 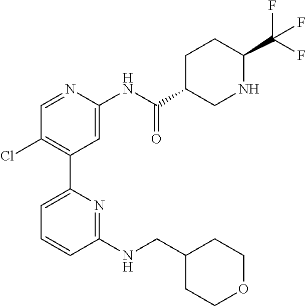 | 498.1 | 0.51 | (3R,6S)-/(3S,6R)-6- Trifluoromethyl- piperidine-3-carboxylic acid {5′-chloro-6- [(tetrahydro-pyran-4- ylmethyl)-amino]- [2,4′]bipyridinyl-2′-yl}- amide |
| 162 | 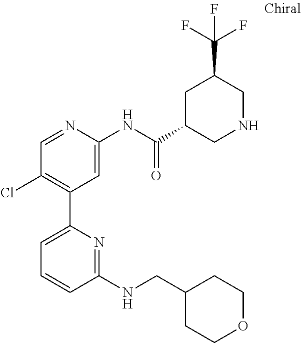 | 498.2 | 0.53 | (3R,5R)-5- Trifluoromethyl- piperidine-3-carboxylic acid {5′-chloro-6- [(tetrahydro-pyran-4- ylmethyl)-amino]- [2,4′]bipyridinyl-2′-yl}- amide |
| 163 | 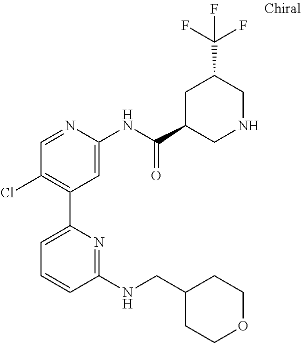 | 498.2 | 0.52 | (3S,5S)-5- Trifluoromethyl- piperidine-3-carboxylic acid {5′-chloro-6- [(tetrahydro-pyran-4- ylmethyl)-amino]- [2,4′]bipyridinyl-2′-yl}- amide |
| 164 | 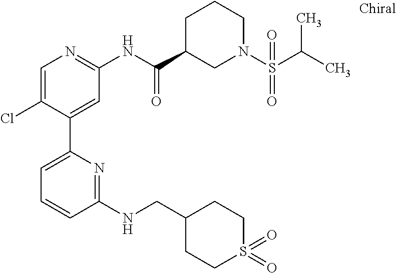 | 584.2 | 0.67 | (S)-1-(Propane-2- sulfonyl)-piperidine-3- carboxylic acid {5′- chloro-6-[(1′,1′-dioxo- hexahydro-1-thiopyran- 4-ylmethyl)-amino]- [2,4′]bipyridinyl-2′-yl}- amide |
| 165 | 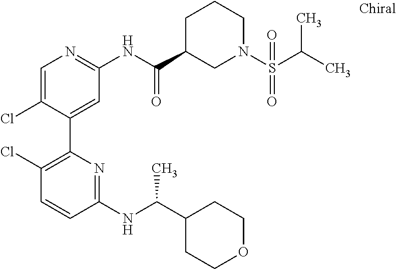 | 584.2 | 0.99 | (S)-1-(Propane-2- sulfonyl)-piperidine-3- carboxylic acid {3,5′- dichloro-6-[(R)-1- (tetrahydro-pyran-4-yl)- ethylamino]- [2,4′]bipyridinyl-2′-yl}- amide |
| 166 | 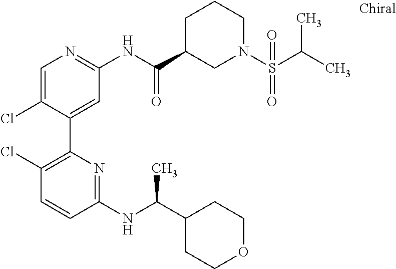 | 584.2 | 0.99 | (S)-1-(Propane-2- sulfonyl)-piperidine-3- carboxylic acid {3,5′- dichloro-6-[(S)-1- (tetrahydro-pyran-4-yl)- ethylamino]- [2,4′]bipyridinyl-2′-yl}- amide |
| 167 | 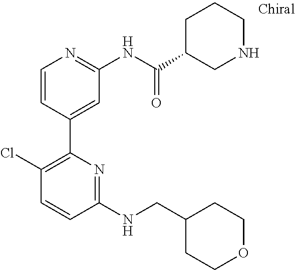 | 430.2 | 0.55 | (R)-Piperidine-3- carboxylic acid {3- chloro-6-[(tetrahydro- pyran-4-ylmethyl)- amino]- [2,4′]bipyridinyl-2′-yl}- amide |
| 168 | 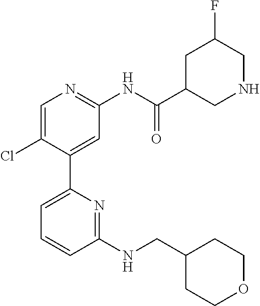 | 448.1 | 0.45 | 5-Fluoro-piperidine-3- carboxylic acid {5′- chloro-6-[(tetrahydro- pyran-4-ylmethyl)- amino]- [2,4′]bipyridinyl-2′-yl}- amide |
| 169 | 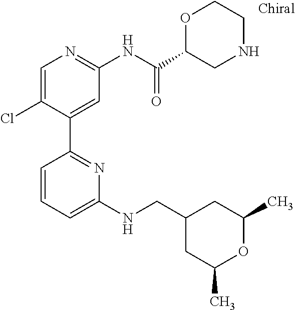 | 460.2 | 0.55 | (R)-Morpholine-2- carboxylic acid {5′- chloro-6-[((2R,6S)-2,6- dimethyl-tetrahydro- pyran-4-ylmethyl)- amino]- [2,4′]bipyridinyl-2′-yl}- amide |
| 170 | 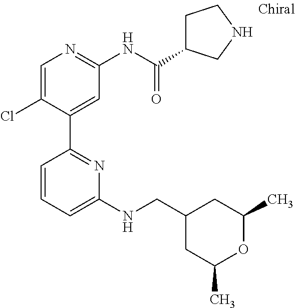 | 444.2 | 0.55 | (R)-Pyrrolidine-3- carboxylic acid {5′- chloro-6-[((2R,6S)-2,6- dimethyl-tetrahydro- pyran-4-ylmethyl)- amino]- [2,4′]bipyridinyl-2′-yl}- amide |
| 171 | 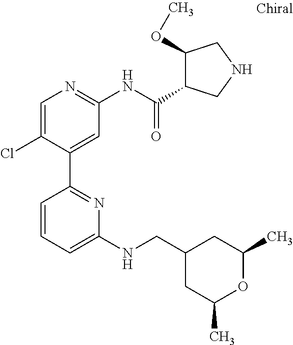 | 474.2 | 0.56 | (3S,4R)-4-Methoxy- pyrrolidine-3- carboxylic acid {5′- chloro-6-[((2R,6S)-2,6- dimethyl-tetrahydro- pyran-4-ylmethyl)- amino]- [2,4′]bipyridinyl-2′-yl}- amide |
| 172 | 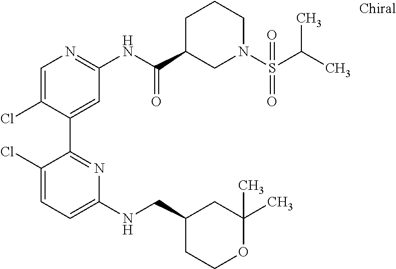 | 598.1/600.1 | 0.97 | (S)-1-(Propane-2- sulfonyl)-piperidine-3- carboxylic acid {3,5′- dichloro-6-[((R)-2,2- dimethyl-tetrahydro- pyran-4-ylmethyl)- amino]- [2,4′]bipyridinyl-2′-yl}- amide |
| 173 | 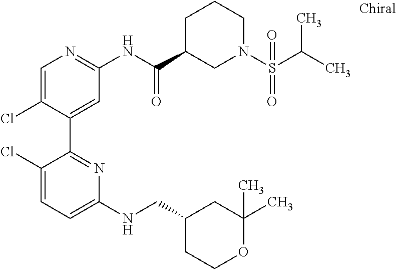 | 598.1/600.1 | 0.97 | (S)-1-(Propane-2- sulfonyl)-piperidine-3- carboxylic acid {3,5′- dichloro-6-[((S)-2,2- dimethyl-tetrahydro- pyran-4-ylmethyl)- amino]- [2,4′]bipyridinyl-2′-yl}- amide |
| 174 | 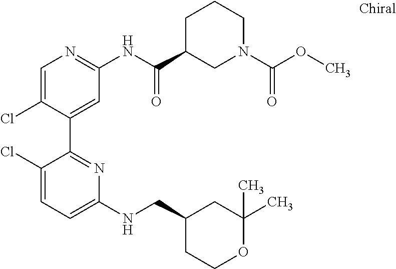 | 550.1/552.1 | 0.93 | (S)-3-{3,5′-Dichloro-6- [((R)-2,2-dimethyl- tetrahydro-pyran-4- ylmethyl)-amino]- [2,4′]bipyridinyl-2′- ylcarbamoyl}- piperidine-1-carboxylic acid methyl ester |
| 175 | 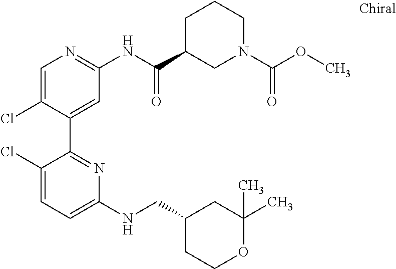 | 550.1/552.1 | 0.93 | (S)-3-{3,5′-Dichloro-6- [((S)-2,2-dimethyl- tetrahydro-pyran-4- ylmethyl)-amino]- [2,4′]bipyridinyl-2′- ylcarbamoyl}- piperidine-1-carboxylic acid methyl ester |
| 176 | 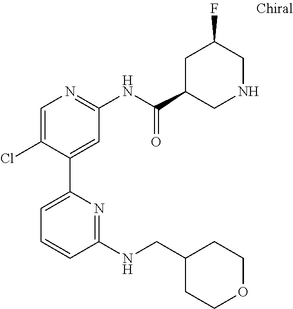 | 448 | 0.46 | (3S,5R)-5-Fluoro- piperidine-3-carboxylic acid {5′-chloro-6- [(tetrahydro-pyran-4- ylmethyl)-amino]- [2,4′]bipyridinyl-2′-yl}- amide |
| 177 | 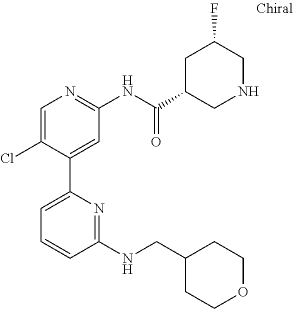 | 448.2 | 0.47 | (3R,5S)-5-Fluoro- piperidine-3-carboxylic acid {5′-chloro-6- [(tetrahydro-pyran-4- ylmethyl)-amino]- [2,4′]bipyridinyl-2′-yl}- amide |
| 178 | 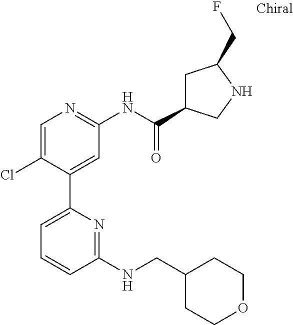 | 448.1 | 0.46 | (3S,5S)-5- Fluoromethyl- pyrrolidine-3- carboxylic acid {5′- chloro-6-[(tetrahydro- pyran-4-ylmethyl)- amino]- [2,4′]bipyridinyl-2′-yl}- amide |
| 179 | 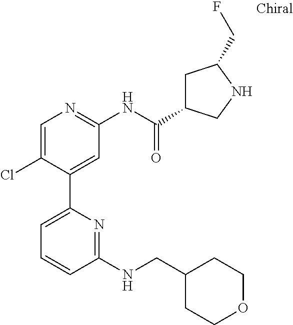 | 448.2 | 0.46 | (3R,5R)-5- Fluoromethyl- pyrrolidine-3- carboxylic acid {5′- chloro-6-[(tetrahydro- pyran-4-ylmethyl)- amino]- [2,4′]bipyridinyl-2′-yl}- amide |
| 180 | 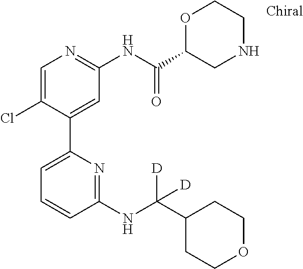 | 434.2 | 0.46 | (R)-Morpholine-2- carboxylic acid {5′- chloro-6-[(tetrahydro- pyran-4- yl(dideuteromethyl))- amino]- [2,4′]bipyridinyl-2′-yl}- amide |
| 181 | 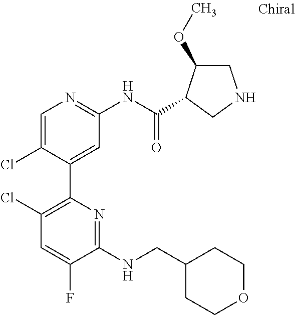 | 498.1 | 0.75 | (3S,4R)-4-Methoxy- pyrrolidine-3- carboxylic acid {3,5′- dichloro-5-fluoro-6- [(tetrahydro-pyran-4- ylmethyl)-amino]- [2,4′]bipyridinyl-2′-yl}- amide |
| 182 | 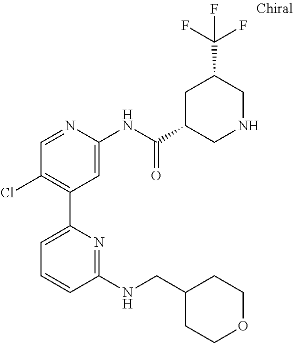 | 498.1 | 0.55 | (3R,5S)-5- Trifluoromethyl- piperidine-3-carboxylic acid {5′-chloro-6- [(tetrahydro-pyran-4- ylmethyl)-amino]- [2,4′]bipyridinyl-2′-yl}- amide |
| 183 | 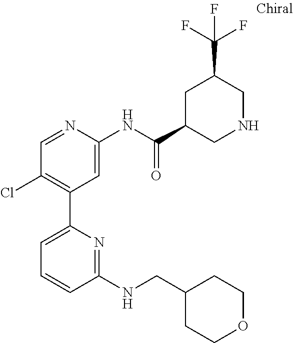 | 498.1 | 0.55 | (3S,5R)-5- Trifluoromethyl- piperidine-3-carboxylic acid {5′-chloro-6- [(tetrahydro-pyran-4- ylmethyl)-amino]- [2,4′]bipyridinyl-2′-yl}- amide |
| 184 | 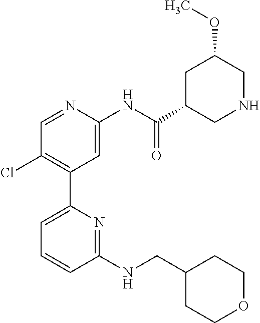 | 460.2 | 0.48 | (3R,5S)-/(3S,5R)-5- Methoxy-piperidine-3- carboxylic acid {5′- chloro-6-[(tetrahydro- pyran-4-ylmethyl)- amino]- [2,4′]bipyridinyl-2′-yl}- amide |
| 185 | 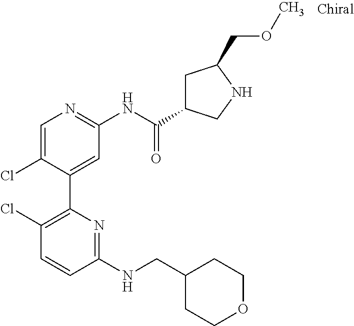 | 494.3/496.1 | 0.64 | (3R,5S)-5- Methoxymethyl- pyrrolidine-3- carboxylic acid {3,5′- dichloro-6-[(tetrahydro- pyran-4-ylmethyl)- amino]- [2,4′]bipyridinyl-2′-yl}- amide |
| 186 | 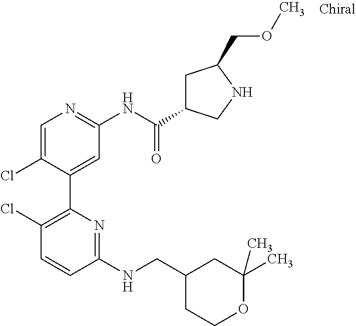 | 522.3/524.2 | 0.75 | (3R,5S)-5- Methoxymethyl- pyrrolidine-3- carboxylic acid {3,5′- dichloro-6-[(2,2- dimethyl-tetrahydro- pyran-4-ylmethyl)- amino]- [2,4′]bipyridinyl-2′-yl}- amide |
| 187 | 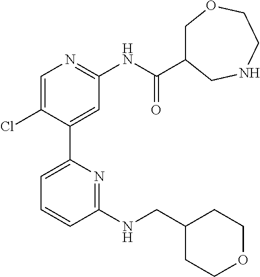 | 446.3/448.3 | 0.46 | [1,4]Oxazepane-6- carboxylic acid {5′- chloro-6-[(tetrahydro- pyran-4-ylmethyl)- amino]- [2,4′]bipyridinyl-2′-yl}- amide |
| 188 | 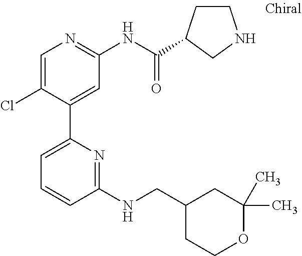 | 444.2 | 0.51 | (R)-Pyrrolidine-3- carboxylic acid {5′- chloro-6-[(2,2- dimethyl-tetrahydro- pyran-4-ylmethyl)- amino]- [2,4′]bipyridinyl-2′-yl}- amide |
| 189 | 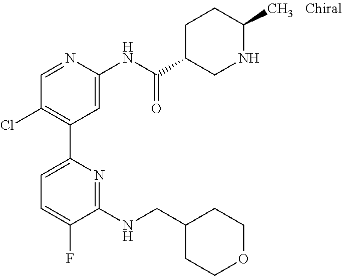 | 462.3 | 0.72 | (3R,6R)-6-Methyl- piperidine-3-carboxylic acid {5′-chloro-5- fluoro-6-[(tetrahydro- pyran-4-ylmethyl)- amino]- [2,4′]bipyridinyl-2′-yl}- amide |
| 190 | 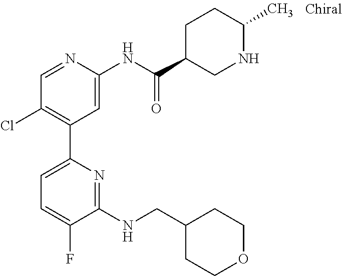 | 462.3 | 0.71 | (3S,6S)-6-Methyl- piperidine-3-carboxylic acid {5′-chloro-5- fluoro-6-[(tetrahydro- pyran-4-ylmethyl)- amino]- [2,4′]bipyridinyl-2′-yl}- amide |
| 191 | 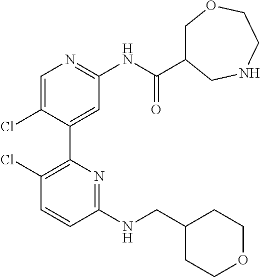 | 480.4/482.2 | 0.65 | [1,4]Oxazepane-6- carboxylic acid {3,5′- dichloro-6-[(tetrahydro- pyran-4-ylmethyl)- amino]- [2,4′]bipyridinyl-2′-yl}- amide |
| 192 | 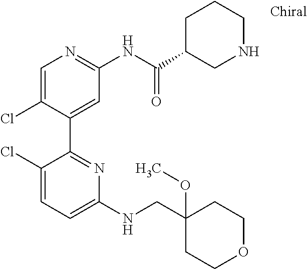 | 494.4 | 0.67 | (R)-Piperidine-3- carboxylic acid {3,5′- dichloro-6-[(4- methoxy-tetrahydro- pyran-4-ylmethyl)- amino]- [2,4′]bipyridinyl-2′-yl}- amide |
| 193 | 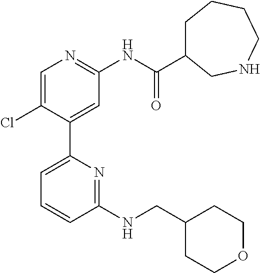 | 444.3 | 0.48 | Azepane-3-carboxylic acid {5′-chloro-6- [(tetrahydro-pyran-4- ylmethyl)-amino]- [2,4′]bipyridinyl-2′-yl}- amide |
| 194 | 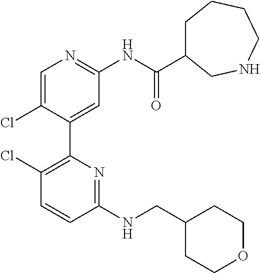 | 478.3/480.2 | 0.72 | Azepane-3-carboxylic acid {3,5′-dichloro-6- [(tetrahydro-pyran-4- ylmethyl)-amino]- [2,4′]bipyridinyl-2′-yl}- amide |
| 195 | 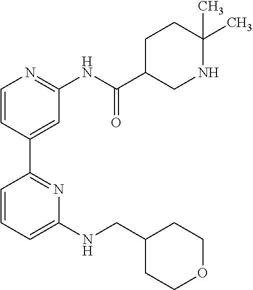 | 424.4 | 0.47 | 6,6-Dimethyl- piperidine-3-carboxylic acid {6-[(tetrahydro- pyran-4-ylmethyl)- amino]- [2,4′]bipyridinyl-2′-yl}- amide |
| 196 | 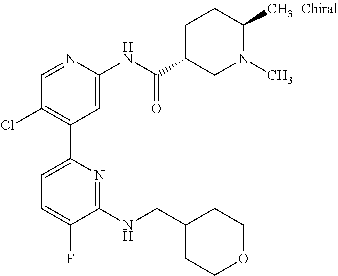 | 476.3 | 0.73 | (3R,6R)-1,6-Dimethyl- piperidine-3-carboxylic acid {5′-chloro-5- fluoro-6-[(tetrahydro- pyran-4-ylmethyl)- amino]- [2,4′]bipyridinyl-2′-yl}- amide |
| 197 | 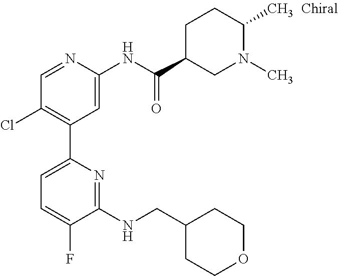 | 476.3 | 0.73 | (3S,6S)-1,6-Dimethyl- piperidine-3-carboxylic acid {5′-chloro-5- fluoro-6-[(tetrahydro- pyran-4-ylmethyl)- amino]- [2,4′]bipyridinyl-2′-yl}- amide |
| 198 | 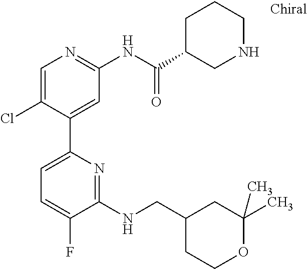 | 476.3 | 0.76 | (R)-Piperidine-3- carboxylic acid {5′- chloro-6-[(2,2- dimethyl-tetrahydro- pyran-4-ylmethyl)- amino]-5-fluoro- [2,4′]bipyridinyl-2′-yl}- amide |
| 199 | 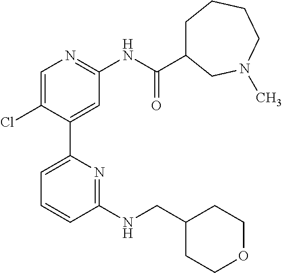 | 458.4 | 0.49 | 1-Methyl-azepane-3- carboxylic acid {5′- chloro-6-[(tetrahydro- pyran-4-ylmethyl)- amino]- [2,4′]bipyridinyl-2′-yl}- amide |
| 200 | 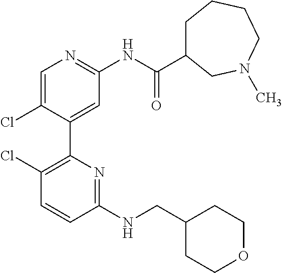 | 492.4 | 0.69 | 1-Methyl-azepane-3- carboxylic acid {3,5′- dichloro-6-[(tetrahydro- pyran-4-ylmethyl)- amino]- [2,4′]bipyridinyl-2′-yl}- amide |
| 201 | 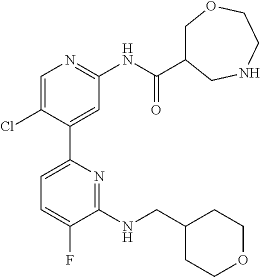 | 464.3 | 0.69 | [1,4]Oxazepane-6- carboxylic acid {5′- chloro-5-fluoro-6- [(tetrahydro-pyran-4- ylmethyl)-amino]- [2,4′]bipyridinyl-2′-yl}- amide |
| 202 | 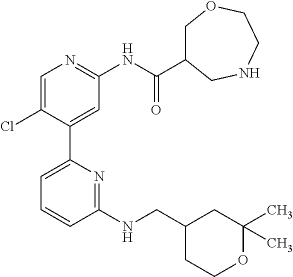 | 474.3 | 0.52 | [1,4]Oxazepane-6- carboxylic acid {5′- chloro-6-[(2,2- dimethyl-tetrahydro- pyran-4-ylmethyl)- amino]- [2,4′]bipyridinyl-2′-yl}- amide |
| 203 | 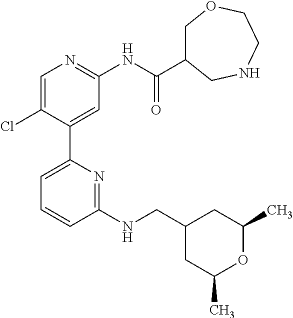 | 474.3 | 0.53 | [1,4]Oxazepane-6- carboxylic acid {5′- chloro-6-[((2R,6S)-2,6- dimethyl-tetrahydro- pyran-4-ylmethyl)- amino]- [2,4′]bipyridinyl-2′-yl}- amide |
| 204 | 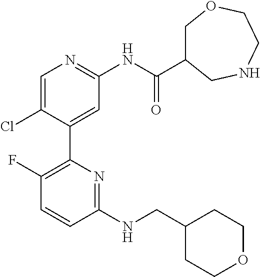 | 464.3 | 0.62 | [1,4]Oxazepane-6- carboxylic acid {5′- chloro-3-fluoro-6- [(tetrahydro-pyran-4- ylmethyl)-amino]- [2,4′]bipyridinyl-2′-yl}- amide |
| 205 | 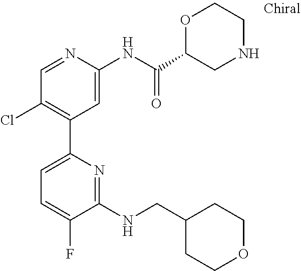 | 450.2 | 0.68 | (R)-Morpholine-2- carboxylic acid {5′- chloro-5-fluoro-6- [(tetrahydro-pyran-4- ylmethyl)-amino]- [2,4′]bipyridinyl-2′-yl}- amide |
| 206 | 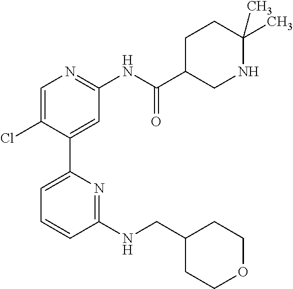 | 458.3 | 0.5 | 6,6-Dimethyl- piperidine-3-carboxylic acid {5′-chloro-6- [(tetrahydro-pyran-4- ylmethyl)-amino]- [2,4′]bipyridinyl-2′-yl}- amide |
| 207 | 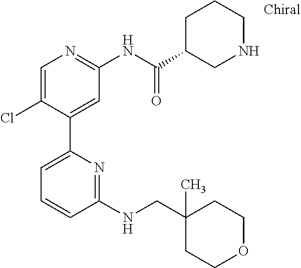 | 444.3 | 0.49 | (R)-Piperidine-3- carboxylic acid {5′- chloro-6-[(4-methyl- tetrahydro-pyran-4- ylmethyl)-amino]- [2,4′]bipyridinyl-2′-yl}- amide |
| 208 | 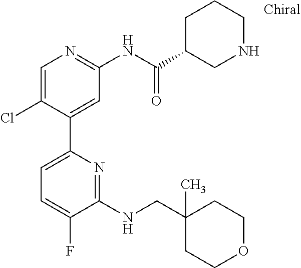 | 462.3 | 0.75 | (R)-Piperidine-3- carboxylic acid {5′- chloro-5-fluoro-6-[(4- methyl-tetrahydro- pyran-4-ylmethyl)- amino]- [2,4′]bipyridinyl-2′-yl}- amide |
| 209 | 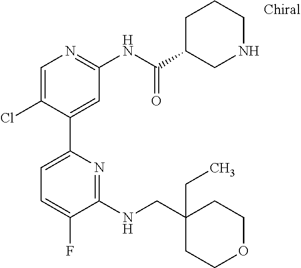 | 476.3 | 0.8 | (R)-Piperidine-3- carboxylic acid {5′- chloro-6-[(4-ethyl- tetrahydro-pyran-4- ylmethyl)-amino]-5- fluoro-[2,4′]bipyridinyl- 2′-yl}-amide |
| 210 | 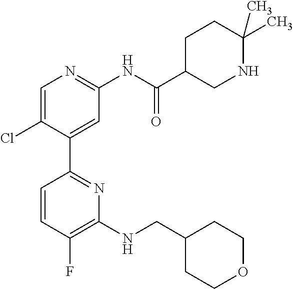 | 476.3 | 0.74 | 6,6-Dimethyl- piperidine-3-carboxylic acid {5′-chloro-5- fluoro-6-[(tetrahydro- pyran-4-ylmethyl)- amino]- [2,4′]bipyridinyl-2′-yl}- amide |
| 211 | 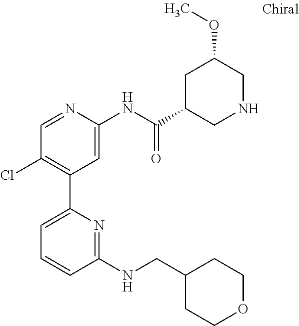 | 460.3 | 0.47 | (3R,5S)-5-Methoxy- piperidine-3-carboxylic acid {5′-chloro-6- [(tetrahydro-pyran-4- ylmethyl)-amino]- [2,4′]bipyridinyl-2′-yl}- amide |
| 212 | 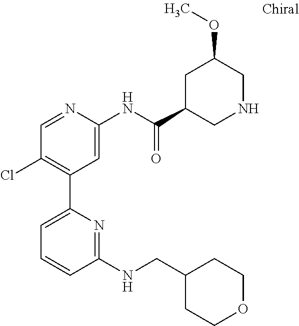 | 460.3 | 0.47 | (3S,5R)-5-Methoxy- piperidine-3-carboxylic acid {5′-chloro-6- [(tetrahydro-pyran-4- ylmethyl)-amino]- [2,4′]bipyridinyl-2′-yl}- amide |
| 213 | 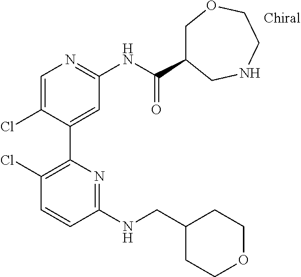 | 480.4/482.2 | 0.65 | (R)-[1,4]Oxazepane-6- carboxylic acid {3,5′- dichloro-6-[(tetrahydro- pyran-4-ylmethyl)- amino]- [2,4′]bipyridinyl-2′-yl}- amide |
| 214 | 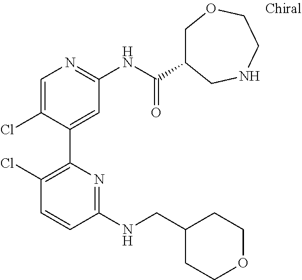 | 480.4/482.2 | 0.65 | (S)-[1,4]Oxazepane-6- carboxylic acid {3,5′- dichloro-6-[(tetrahydro- pyran-4-ylmethyl)- amino]- [2,4′]bipyridinyl-2′-yl}- amide |
| 215 | 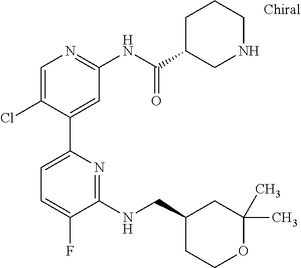 | 476.2 | 0.75 | (R)-Piperidine-3- carboxylic acid {5′- chloro-6-[((R)-2,2- dimethyl-tetrahydro- pyran-4-ylmethyl)- amino]-5-fluoro- [2,4′]bipyridinyl-2′-yl}- amide |
| 216 | 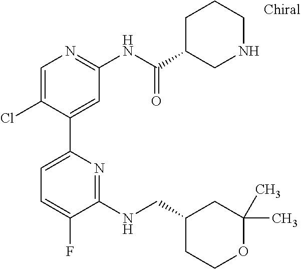 | 476.2 | 0.75 | (R)-Piperidine-3- carboxylic acid {5′- chloro-6-[((S)-2,2- dimethyl-tetrahydro- pyran-4-ylmethyl)- amino]-5-fluoro- [2,4′]bipyridinyl-2′-yl}- amide |
| 217 | 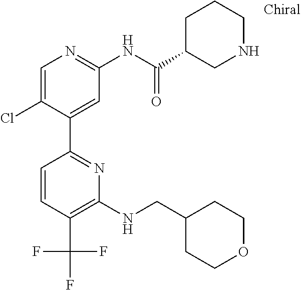 | 498.3 | 0.83 | (R)-Piperidine-3- carboxylic acid {5′- chloro-6-[(tetrahydro- pyran-4-ylmethyl)- amino]-5- trifluoromethyl- [2,4′]bipyridinyl-2′-yl}- amide |
| 218 | 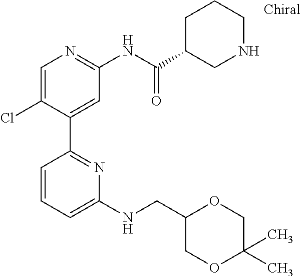 | 460.3 | 0.51 | (R)-Piperidine-3- carboxylic acid {5′- chloro-6-[(5,5- dimethyl-[1,4]dioxan- 2-ylmethyl)-amino]- [2,4′]bipyridinyl-2′-yl}- amide |
| 219 | 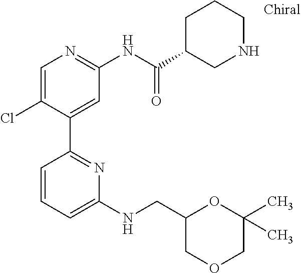 | 460.3 | 0.51 | (R)-Piperidine-3- carboxylic acid {5′- chloro-6-[(6,6- dimethyl-[1,4]dioxan- 2-ylmethyl)-amino]- [2,4′]bipyridinyl-2′-yl}- amide |
| 220 | 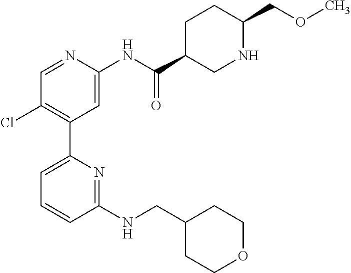 | 474.3 | 0.50 | (3S,6S)-/(3R,6R)-6- Methoxymethyl- piperidine-3-carboxylic acid {5′-chloro-6- [(tetrahydro-pyran-4- ylmethyl)-amino]- [2,4′]bipyridinyl-2′-yl}- amide |
| 221 | 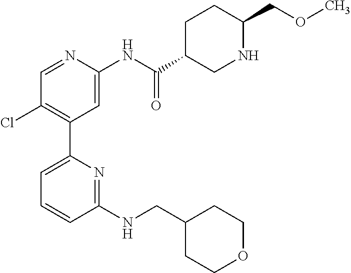 | 474.3 | 0.47 | (3R,6S)-/(3S,6R)-6- Methoxymethyl- piperidine-3-carboxylic acid {5′-chloro-6- [(tetrahydro-pyran-4- ylmethyl)-amino]- [2,4′]bipyridinyl-2′-yl}- amide |
| 222 | 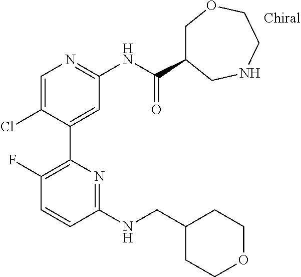 | 464.4 | 0.56 | (R)-[1,4]Oxazepane-6- carboxylic acid {5′- chloro-3-fluoro-6- [(tetrahydro-pyran-4- ylmethyl)-amino]- [2,4′]bipyridinyl-2′-yl}- amide |
| 223 | 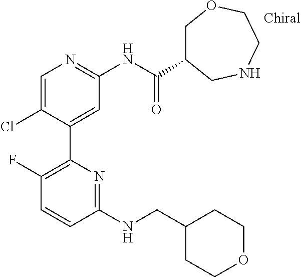 | 464.4 | 0.6 | (S)-[1,4]Oxazepane-6- carboxylic acid {5′- chloro-3-fluoro-6- [(tetrahydro-pyran-4- ylmethyl)-amino]- [2,4′]bipyridinyl-2′-yl}- amide |
| 224 | 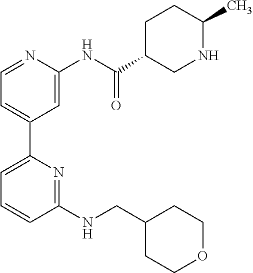 | 410.3 | 0.48 | (3R,6R)-/(3S,6S)-6- Methyl-piperidine-3- carboxylic acid {6- [(tetrahydro-pyran-4- ylmethyl)-amino]- [2,4′]bipyridinyl-2′-yl}- amide |
| 225 | 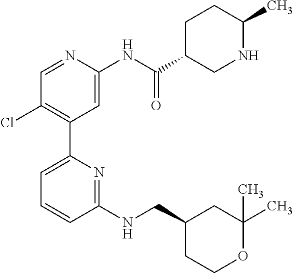 | 472.3 | 0.54 | (3R,6R)-/(3S,6S)-6- Methyl-piperidine-3- carboxylic acid {5′- chloro-6-[((R)-2,2- dimethyl-tetrahydro- pyran-4-ylmethyl)- amino]- [2,4′]bipyridinyl-2′-yl}- amide |
| 226 | 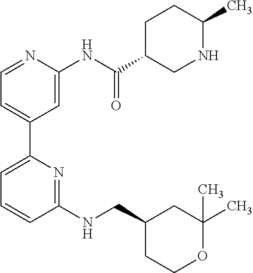 | 438.3 | 0.5 | (3R,6R)-/(3S,6S)-6- Methyl-piperidine-3- carboxylic acid {6- [((R)-2,2-dimethyl- tetrahydro-pyran-4- ylmethyl)-amino]- [2,4′]bipyridinyl-2′-yl}- amide |
| 227 | 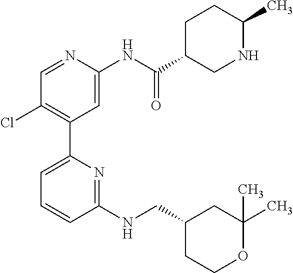 | 472.3 | 0.54 | (3R,6R)-/(3S,6S)-6- Methyl-piperidine-3- carboxylic acid {5′- chloro-6-[((S)-2,2- dimethyl-tetrahydro- pyran-4-ylmethyl)- amino]- [2,4′]bipyridinyl-2′-yl}- amide |
| 228 | 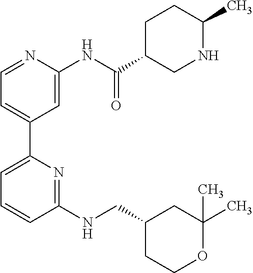 | 438.3 | 0.5 | (3R,6R)-/(3S,6S)-6- Methyl-piperidine-3- carboxylic acid {6- [((S)-2,2-dimethyl- tetrahydro-pyran-4- ylmethyl)-amino]- [2,4′]bipyridinyl-2′-yl}- amide |
| 229 | 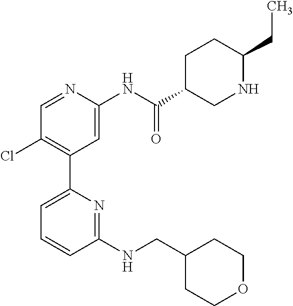 | 458.2 | 0.51 | (3R,6R)-/(3S,6S)-6- Ethyl-piperidine-3- carboxylic acid {5′- chloro-6-[(tetrahydro- pyran-4-ylmethyl)- amino]- [2,4′]bipyridinyl-2′-yl}- amide |
| 230 | 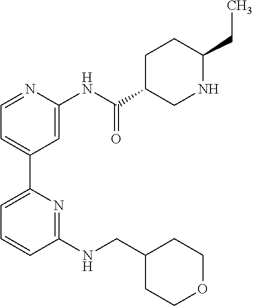 | 424.3 | 0.47 | (3R,6R)-/(3S,6S)-6- Ethyl-piperidine-3- carboxylic acid {6- [(tetrahydro-pyran-4- ylmethyl)-amino]- [2,4′]bipyridinyl-2′-yl}- amide |
| 231 | 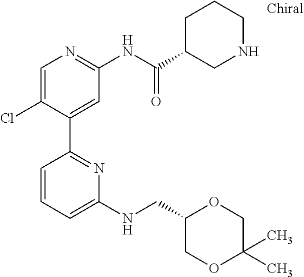 | 460.1 | 0.52 | (R)-Piperidine-3- carboxylic acid {5′- chloro-6-[((S)-5,5- dimethyl-[1,4]dioxan- 2-ylmethyl)-amino]- [2,4′]bipyridinyl-2′-yl}- amide |
| 232 | 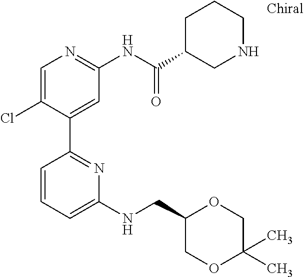 | 460.2 | 0.52 | (R)-Piperidine-3- carboxylic acid {5′- chloro-6-[((R)-5,5- dimethyl-[1,4]dioxan- 2-ylmethyl)-amino]- [2,4′]bipyridinyl-2′-yl}- amide |
| 233 | 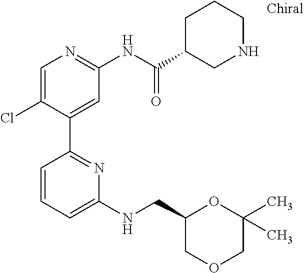 | 460.1 | 0.6 | (R)-Piperidine-3- carboxylic acid {5′- chloro-6-[((R)-6,6- dimethyl-[1,4]dioxan-2- ylmethyl)-amino]- [2,4′]bipyridinyl-2′-yl}- amide |
| 234 | 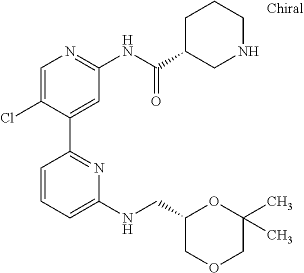 | 460.1 | 0.59 | (R)-Piperidine-3- carboxylic acid {5′- chloro-6-[((S)-6,6- dimethyl-[1,4]dioxan- 2-ylmethyl)-amino]- [2,4′]bipyridinyl-2′-yl}- amide |
| 235 | 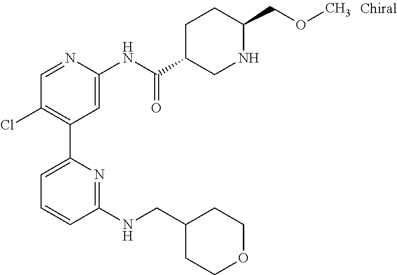 | 474.3 | 0.47 | (3R,6S)-6- Methoxymethyl- piperidine-3-carboxylic acid {5′-chloro-6- [(tetrahydro-pyran-4- ylmethyl)-amino]- [2,4′]bipyridinyl-2′-yl}- amide |
| 236 | 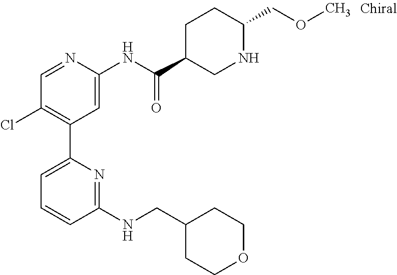 | 474.3 | 0.47 | (3S,6R)-6- Methoxymethyl- piperidine-3-carboxylic acid {5′-chloro-6- [(tetrahydro-pyran-4- ylmethyl)-amino]- [2,4′]bipyridinyl-2′-yl}- amide |
| 237 | 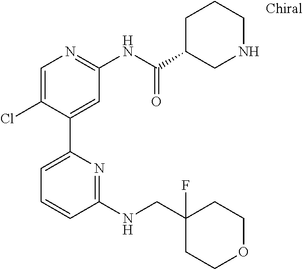 | 448.1 | 0.52 | (R)-Piperidine-3- carboxylic acid {5′- chloro-6-[(4-fluoro- tetrahydro-pyran-4- ylmethyl)-amino]- [2,4′]bipyridinyl-2′-yl}- amide |
| 238 | 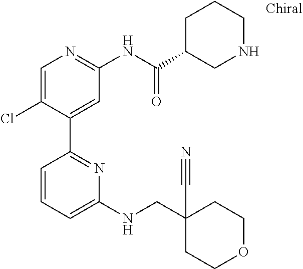 | 455 | 0.55 | (R)-Piperidine-3- carboxylic acid {5′- chloro-6-[(4-cyano- tetrahydro-pyran-4- ylmethyl)-amino]- [2,4′]bipyridinyl-2′-yl}- amide |
| 239 | 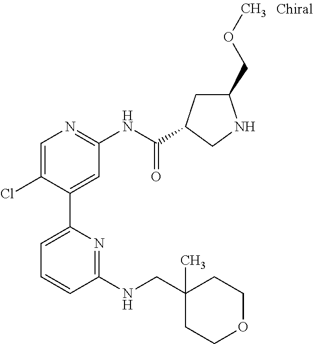 | 474.1 | 0.5 | (3R,5S)-5- Methoxymethyl- pyrrolidine-3- carboxylic acid {5′- chloro-6-[(4-methyl- tetrahydro-pyran-4- ylmethyl)-amino]- [2,4′]bipyridinyl-2′-yl}- amide |
| 240 | 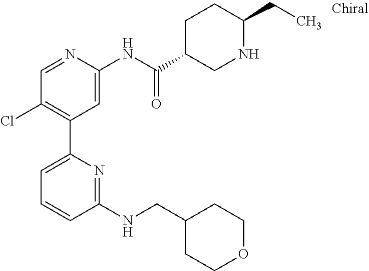 | 458.1 | 0.49 | (3R,6R)-6-Ethyl- piperidine-3-carboxylic acid {5′-chloro-6- [(tetrahydro-pyran-4- ylmethyl)-amino]- [2,4′]bipyridinyl-2′-yl}- amide |
| 241 | 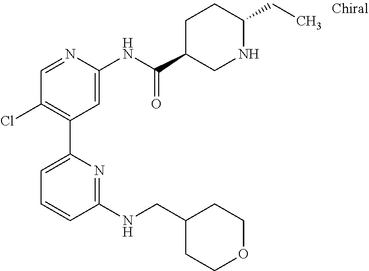 | 458.1 | 0.49 | (3S,6S)-6-Ethyl- piperidine-3-carboxylic acid {5′-chloro-6- [(tetrahydro-pyran-4- ylmethyl)-amino]- [2,4′]bipyridinyl-2′-yl}- amide |
| 242 | 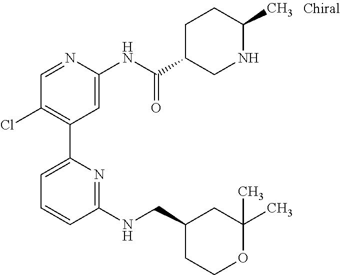 | 472.1 | 0.53 | (3R,6R)-6-Methyl- piperidine-3-carboxylic acid {5′-chloro-6-[((R)- 2,2-dimethyl- tetrahydro-pyran-4- ylmethyl)-amino]- [2,4′]bipyridinyl-2′-yl}- amide |
| 243 | 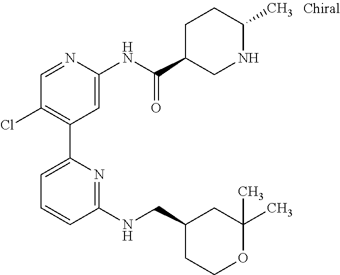 | 472.1 | 0.53 | (3S,6S)-6-Methyl- piperidine-3-carboxylic acid {5′-chloro-6-[((R)- 2,2-dimethyl- tetrahydro-pyran-4- ylmethyl)-amino]- [2,4′]bipyridinyl-2′-yl}- amide |
| 244 | 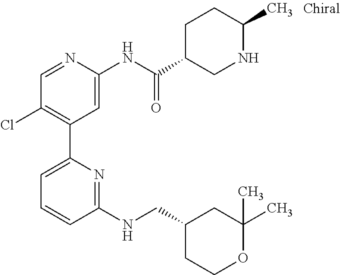 | 472.1 | 0.52 | (3R,6R)-6-Methyl- piperidine-3-carboxylic acid {5′-chloro-6-[((S)- 2,2-dimethyl- tetrahydro-pyran-4- ylmethyl)-amino]- [2,4′]bipyridinyl-2′-yl}- amide |
| 245 | 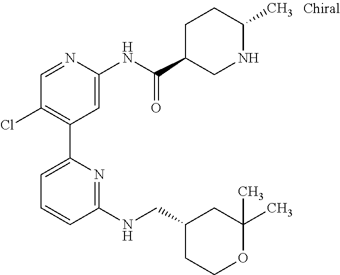 | 472.1 | 0.52 | (3S,6S)-6-Methyl- piperidine-3-carboxylic acid {5′-chloro-6-[((S)- 2,2-dimethyl- tetrahydro-pyran-4- ylmethyl)-amino]- [2,4′]bipyridinyl-2′-yl}- amide |
| 246 | 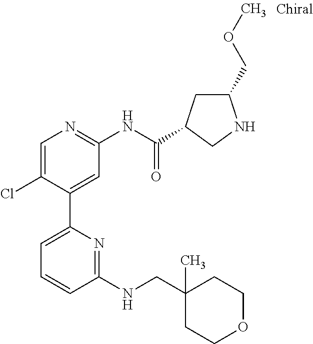 | 474.2 | 0.51 | (3R,5R)-5- Methoxymethyl- pyrrolidine-3- carboxylic acid {5′- chloro-6-[(4-methyl- tetrahydro-pyran-4- ylmethyl)-amino]- [2,4′]bipyridinyl-2′-yl}- amide |
| 247 | 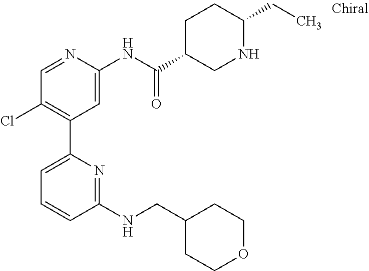 | 458.1 | 0.5 | (3R,6S)-6-Ethyl- piperidine-3-carboxylic acid {5′-chloro-6- [(tetrahydro-pyran-4- ylmethyl)-amino]- [2,4′]bipyridinyl-2′-yl}- amide |
| 248 | 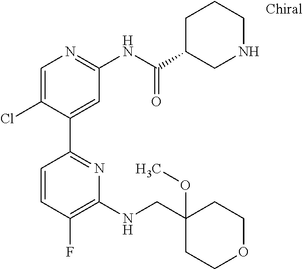 | 478 | 0.69 | (R)-Piperidine-3- carboxylic acid {5′- chloro-5-fluoro-6-[(4- methoxy-tetrahydro- pyran-4-ylmethyl)- amino]- [2,4′]bipyridinyl-2′-yl}- amide |
| 249 | 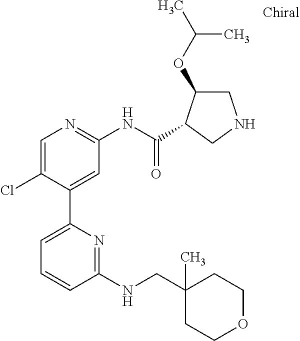 | 488 | 0.55 | (3S,4R)-4-Isopropoxy- pyrrolidine-3- carboxylic acid {5′- chloro-6-[(4-methyl- tetrahydro-pyran-4- ylmethyl)-amino]- [2,4′]bipyridinyl-2′-yl}- amide |
| 250 | 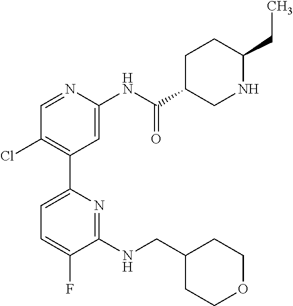 | 476.2 | 0.74 | (3R,6R)-/(3S,6S)-6- Ethyl-piperidine-3- carboxylic acid {5′- chloro-5-fluoro-6- [(tetrahydro-pyran-4- ylmethyl)-amino]- [2,4′]bipyridinyl-2′-yl}- amide |
| 251 | 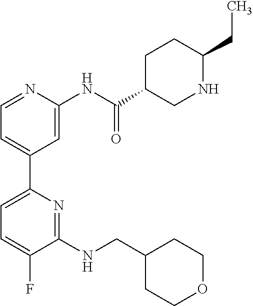 | 442.3 | 0.58 | (3R,6R)-/(3S,6S)-6- Ethyl-piperidine-3- carboxylic acid {5- fluoro-6-[(tetrahydro- pyran-4-ylmethyl)- amino]- [2,4′]bipyridinyl-2′-yl}- amide |
| 252 | 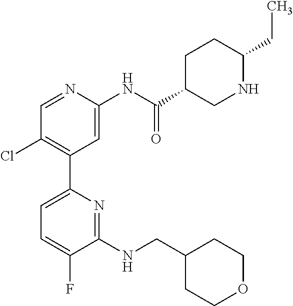 | 476.2 | 0.77 | (3R,6S)-/(3S,6R)-6- Ethyl-piperidine-3- carboxylic acid {5′- chloro-5-fluoro-6- [(tetrahydro-pyran-4- ylmethyl)-amino]- [2,4′]bipyridinyl-2′-yl}- amide |
| 253 | 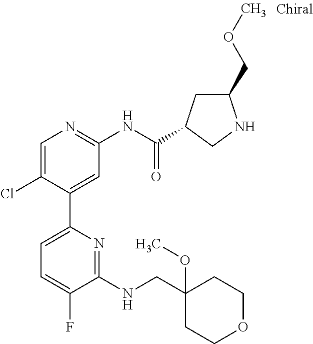 | 508 | 0.69 | (3R,5S)-5- Methoxymethyl- pyrrolidine-3- carboxylic acid {5′- chloro-5-fluoro-6-[(4- methoxy-tetrahydro- pyran-4-ylmethyl)- amino]- [2,4′]bipyridinyl-2′-yl}- amide |
| 254 | 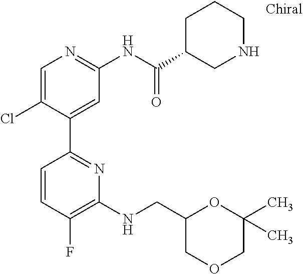 | 478.2 | 0.74 | (R)-Piperidine-3- carboxylic acid {5′- chloro-6-[(6,6- dimethyl-[1,4]dioxan- 2-ylmethyl)-amino]-5- fluoro-[2,4′]bipyridinyl- 2′-yl}-amide |
| 255 | 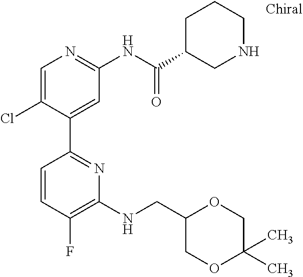 | 478.2 | 0.73 | (R)-Piperidine-3- carboxylic acid {5′- chloro-6-[(5,5- dimethyl-[1,4]dioxan- 2-ylmethyl)-amino]-5- fluoro-[2,4′]bipyridinyl- 2′-yl}-amide |
| 256 | 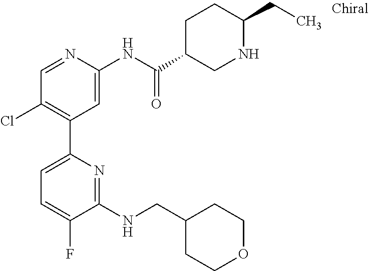 | 476.2 | 0.75 | (3R,6R)-6-Ethyl- piperidine-3-carboxylic acid {5′-chloro-5- fluoro-6-[(tetrahydro- pyran-4-ylmethyl)- amino]- [2,4′]bipyridinyl-2′-yl}- amide |
| 257 | 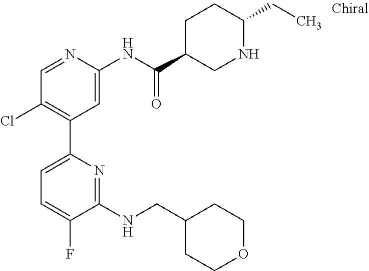 | 476.2 | 0.75 | (3S,6S)-6-Ethyl- piperidine-3-carboxylic acid {5′-chloro-5- fluoro-6-[(tetrahydro- pyran-4-ylmethyl)- amino]- [2,4′]bipyridinyl-2′-yl}- amide |
| 258 | 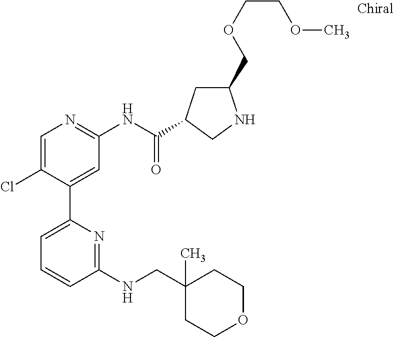 | 518.2 | 0.52 | (3R,5S)-5-(2-Methoxy- ethoxymethyl)- pyrrolidine-3- carboxylic acid {5′- chloro-6-[(4-methyl- tetrahydro-pyran-4- ylmethyl)-amino]- [2,4′]bipyridinyl-2′-yl}- amide |
| 259 | 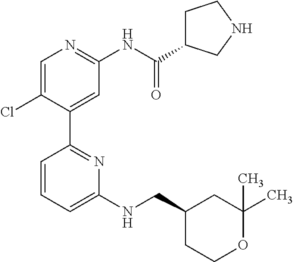 | 444.2 | 0.518 | (R)-Pyrrolidine-3- carboxylic acid {5′- chloro-6-[((R)-2,2- dimethyl-tetrahydro- pyran-4-ylmethyl)- amino]- [2,4′]bipyridinyl-2′-yl}- amide |
| 260 | 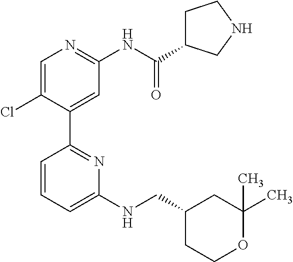 | 444.2 | 0.515 | (R)-Pyrrolidine-3- carboxylic acid {5′- chloro-6-[((S)-2,2- dimethyl-tetrahydro- pyran-4-ylmethyl)- amino]- [2,4′]bipyridinyl-2′-yl}- amide |
| 261 | 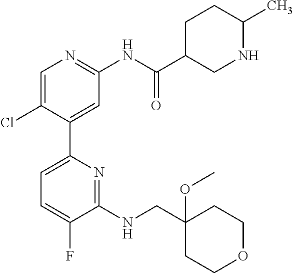 | 492.2 | 0.70 | 6-Methyl-piperidine-3- carboxylic acid {5′- chloro-5-fluoro-6-[(4- methoxy-tetrahydro- pyran-4-ylmethyl)- amino]- [2,4′]bipyridinyl-2′-yl}- amide |
| 262 | 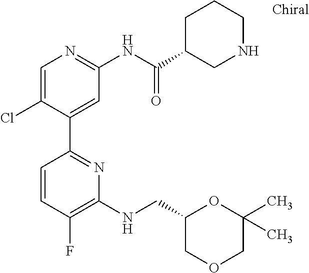 | 478.1 | 0.74 | (R)-Piperidine-3- carboxylic acid {5′- chloro-6-[((S)-6,6- dimethyl-[1,4]dioxan- 2-ylmethyl)-amino]-5- fluoro-[2,4′]bipyridinyl- 2′-yl}-amide |
| 263 | 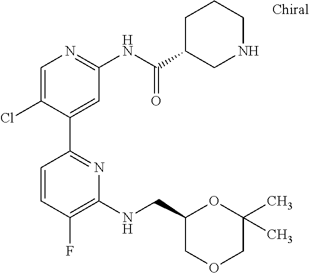 | 478.1 | 0.73 | (R)-Piperidine-3- carboxylic acid {5′- chloro-6-[((R)-6,6- dimethyl-[1,4]dioxan-2- ylmethyl)-amino]-5- fluoro-[2,4′]bipyridinyl- 2′-yl}-amide |
| 264 | 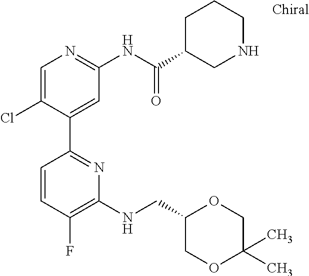 | 478.1 | 0.72 | (R)-Piperidine-3- carboxylic acid {5′- chloro-6-[((S)-5,5- dimethyl-[1,4]dioxan- 2-ylmethyl)-amino]-5- fluoro-[2,4′]bipyridinyl- 2′-yl}-amide |
| 265 | 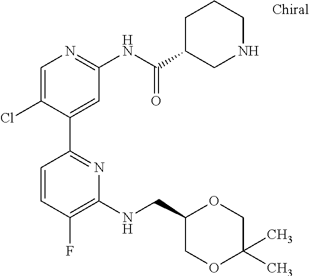 | 478.1 | 0.72 | (R)-Piperidine-3- carboxylic acid {5′- chloro-6-[((R)-5,5- dimethyl-[1,4]dioxan- 2-ylmethyl)-amino]-5- fluoro-[2,4′]bipyridinyl- 2′-yl}-amide |

TABLE II

| | | | Retention | |
|---|---|---|---|---|
| Example | | | Time | |
| No. | Structure | M + H | [min] | Name |
| 301 | 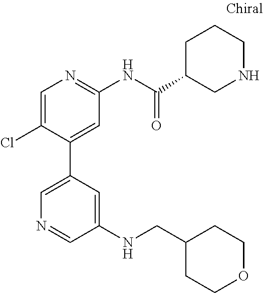 | 430.1 | | (R)-Piperidine- 3-carboxylic acid {5′-chloro- 5-[(tetrahydro- pyran-4- ylmethyl)- amino]- [3,4′]bipyridinyl- 2′-yl}-amide |
| 302 | 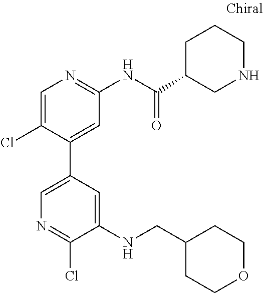 | 464.2 | 0.51 | (R)-Piperidine- 3-carboxylic acid {6,5′- dichloro-5- [(tetrahydro- pyran-4- ylmethyl)- amino]- [3,4′]bipyridinyl- 2′-yl}-amide |
| 303 | 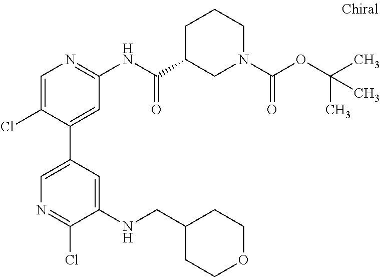 | 564.3 | 1.07 | (R)-3-{6,5′- Dichloro-5- [(tetrahydro- pyran-4- ylmethyl)- amino]- [3,4′]bipyridinyl- 2′-ylcarbamoyl}- piperidine-1- carboxylic acid tert-butyl ester |
| 304 | 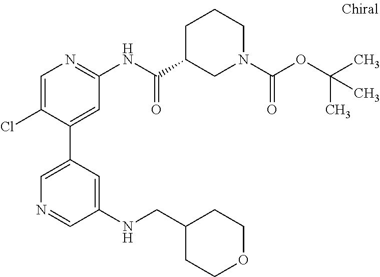 | 530.3 | 0.76 | (R)-3-{5′- Chloro-5- [(tetrahydro- pyran-4- ylmethyl)- amino]- [3,4′]bipyridinyl- 2′-ylcarbamoyl}- piperidine-1- carboxylic acid tert-butyl ester |
| 305 | 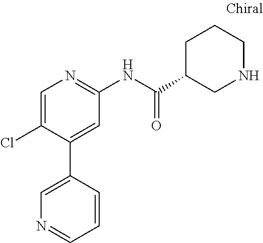 | 317.1 | 0.36 | (R)-Piperidine- 3-carboxylic acid (5′-chloro- [3,4′]bipyridinyl- 2′-yl)-amide |
| 306 | 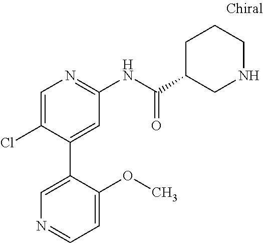 | 347.1 | 0.36 | (R)-Piperidine- 3-carboxylic acid (5′-chloro- 4-methoxy- [3,4′]bipyridinyl- 2′-yl)-amide |
| 307 | 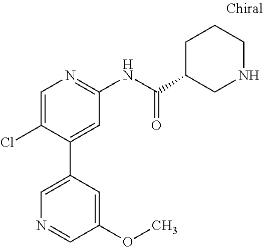 | 347.1 | 0.43 | (R)-Piperidine- 3-carboxylic acid (5′-chloro- 5-methoxy- [3,4′]bipyridinyl- 2′-yl)-amide |
| 308 | 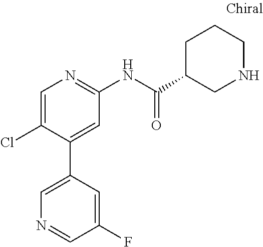 | 335.1 | 0.54 | (R)-Piperidine- 3-carboxylic acid (5′-chloro- 5-fluoro- [3,4′]bipyridinyl- 2′-yl)-amide |
| 309 | 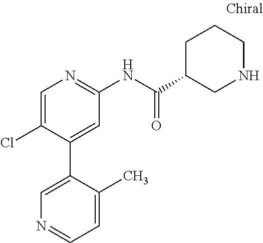 | 331.1 | 0.37 | (R)-Piperidine- 3-carboxylic acid (5′-chloro- 4-methyl- [3,4′]bipyridinyl- 2′-yl)-amide |
| 310 | 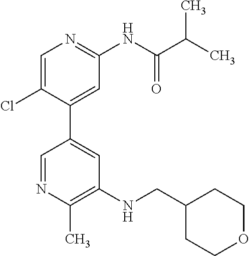 | 403.2 | 0.64 | N-{5′-Chloro-6- methyl-5- [(tetrahydro- pyran-4- ylmethyl)- amino]- [3,4′]bipyridinyl- 2′-yl}- isobutyramide |
| 311 | 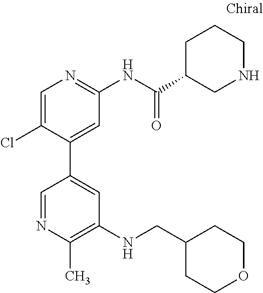 | 444.2 | 0.47 | (R)-Piperidine- 3-carboxylic acid {5′-chloro- 6-methyl-5- [(tetrahydro- pyran-4- ylmethyl)- amino]- [3,4′]bipyridinyl- 2′-yl}-amide |
| 312 | 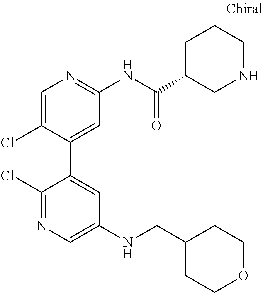 | 464.2 | 0.64 | (R)-Piperidine- 3-carboxylic acid {2,5′- dichloro-5- [(tetrahydro- pyran-4- ylmethyl)- amino]- [3,4′]bipyridinyl- 2′-yl}-amide |
| 313 | 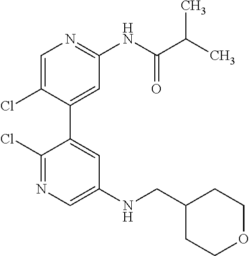 | 423.1 | 0.86 | N-{2,5′- Dichloro-5- [(tetrahydro- pyran-4- ylmethyl)- amino]- [3,4′]bipyridinyl- 2′-yl}- isobutyramide |
| 314 | 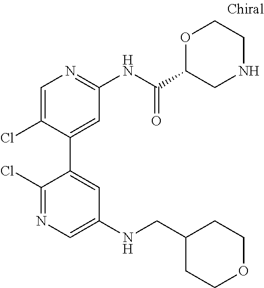 | 466.2 | 0.63 | (R)-Morpholine- 2-carboxylic acid {2,5′- dichloro-5- [(tetrahydro- pyran-4- ylmethyl)- amino]- [3,4′]bipyridinyl- 2′-yl}-amide |
| 315 | 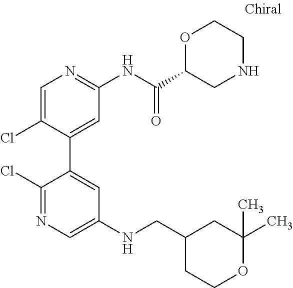 | 494.3 | 0.7 | (R)-Morpholine- 2-carboxylic acid {2,5′- dichloro-5-[(2,2- dimethyl- tetrahydro- pyran-4- ylmethyl)- amino]- [3,4′]bipyridinyl- 2′-yl}-amide |
| 316 | 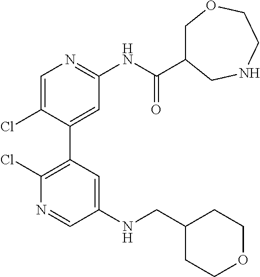 | 480/482 | 0.62 | [l,4]Oxazepane- 6-carboxylic acid {2,5′-dichloro-5- [(tetrahydro- pyran-4- ylmethyl)-amino]- [3,4′]bipyridinyl- 2′-yl}-amide |

Table III, below, provides ¹H NMR data for representative compounds.

TABLE III

| Example | |
|---|---|
| No. | ¹H-NMR |
| 1 | ¹H NMR (400 MHz, methanol-d4) δ [ppm] 1.49-1.66 (m, 1 H) |
| | 1.68-1.82 (m, 2 H) 1.93-2.08 (m, 1 H) 2.56-2.73 (m, 2 H) 2.85 (dd, J = 12.33, 9.59 Hz, |
| | 1 H) 2.91-3.01 (m, 1 H) 3.11 (dd, J = 12.33, 2.54 Hz, 1 H) 4.58 (s, 2 H) |
| | 6.57 (d, J = 8.22 Hz, 1 H) 6.89 (d, J = 7.43 Hz, 1 H) 6.91-6.95 (m, 1 H) |
| | 7.10 (d, J = 10.17 Hz, 1 H) 7.19 (d, J = 7.43 Hz, 1 H) 7.25-7.34 (m, 1 H) |
| | 7.48-7.55 (m, 1 H) 8.32 (s, 2 H) |
| 3 | ¹H NMR (400 MHz, methanol-d4) δ [ppm] 1.21-1.43 (m, 2 H) |
| | 1.48-1.66 (m, 1 H) 1.66-1.86 (m, 4 H) 1.88-2.12 (m, 2 H) 2.59-2.75 (m, 2 H) |
| | 2.88 (dd, J = 12.52, 9.39 Hz, 1 H) 2.93-3.02 (m, 1 H) 3.12 (dd, J = 12.52, 3.52 Hz, |
| | 1 H) 3.25 (d, J = 6.65 Hz, 2 H) 3.37-3.51 (m, 2 H) 3.95 (dd, J = 11.15, 3.33 Hz, |
| | 2 H) 6.54 (d, J = 8.22 Hz, 1 H) 6.86 (d, J = 7.43 Hz, 1 H) 7.39-7.55 (m, 1 |
| | H) 8.33 (s, 1 H) 8.40 (s, 1 H) |
| 12 | ¹H NMR (400 MHz, methanol-d4) δ [ppm] 1.73-2.05 (m, 5 H) |
| | 2.08-2.39 (m, 4 H) 2.98-3.18 (m, 5 H) 3.18-3.27 (m, 3 H) 3.33-3.50 (m, 3 H) |
| | 6.75 (d, J = 9.0 Hz, 1 H) 7.01 (d, J = 9.0 Hz, 1 H) 7.64 (m, 1 H) 8.41 (s, 1 H) 8.49 (s, |
| | 1 H) |
| 17 | ¹H NMR (300 MHz, methanol-d4) δ [ppm] 0.95 (t, J = 11.72 Hz, 2 H) |
| | 1.08-1.33 (m, 3 H) 1.50-1.96 (m, 10 H) 1.98-2.14 (m, 1 H) 2.95 (td, J = 7.69, |
| | 3.66 Hz, 1 H) 3.04 (dd, J = 8.64, 3.37 Hz, 1 H) 3.13 (d, J = 6.74 Hz, 2 H) |
| | 3.23-3.33 (m, 2 H) 6.82 (dd, J = 15.09, 8.06 Hz, 2 H) 7.70 (dd, J = 8.79, 7.33 Hz, 1 |
| | H) 8.26 (s, 1 H) 8.36 (s, 1 H) |
| 30 | ¹H NMR (300 MHz, methanol-d4) δ [ppm] 1.08-1.37 (m, 2 H) |
| | 1.77-2.05 (m, 7 H) 2.09 (s, 3 H) 2.11-2.23 (m, 1 H) 2.58-2.73 (m, 1 H) 3.03 (dd, |
| | J = 7.33, 3.81 Hz, 1 H) 3.05-3.19 (m, 2 H) 3.25 (d, J = 4.10 Hz, 1 H) |
| | 3.27-3.33 (dMeOH, 1 H App.) 3.33-3.37 (m, 2 H) 3.95 (d, J = 13.77 Hz, 1 H) |
| | 4.54 (d, J = 13.48 Hz, 1 H) 6.83-6.99 (m, 2 H) 7.70-7.84 (m, 1 H) 8.39 (s, 1 H) |
| | 8.44 (s, 1 H) |
| 34 | ¹H NMR (300 MHz, methanol-d4) δ [ppm] 1.28-1.47 (m, 1 H) |
| | 1.49-1.74 (m, 4 H) 1.75-2.04 (m, 4 H) 2.14 (d, J = 5.86 Hz, 1 H) 2.96-3.20 (m, 2 H) |
| | 3.24 (d, J = 3.52 Hz, 1 H) 3.35 (br. s., 2 H) 3.37-3.44 (m, 1 H) |
| | 3.45-3.55 (m, 2 H) 3.61 (td, J = 7.84, 2.49 Hz, 1 H) 3.95 (dd, J = 11.28, 2.78 Hz, 1 H) |
| | 6.88-7.03 (m, 2 H) 7.82 (dd, J = 8.79, 7.33 Hz, 1 H) 8.37 (s, 1 H) 8.47 (s, 1 |
| | H) |
| 35 | ¹H NMR (300 MHz, methanol-d4) δ [ppm] 1.37-1.66 (m, 4 H) 1.82 (d, |
| | J = 6.74 Hz, 1 H) 1.88-2.04 (m, 2 H) 2.03-2.25 (m, 2 H) 2.98-3.08 (m, 1 |
| | H) 3.12 (s, 3 H) 3.15-3.22 (m, 2 H) 3.28-3.34 (dMeOH, 1 H App.) |
| | 3.35-3.48 (m, 3 H) 3.59 (d, J = 7.33 Hz, 2 H) 3.90-4.02 (m, 2 H) 6.74 (d, J = 8.50 Hz, |
| | 1 H) 7.00 (d, J = 7.33 Hz, 1 H) 7.65 (t, J = 8.06 Hz, 1 H) 8.37 (s, 1 H) |
| | 8.50 (s, 1 H) |
| 41 | ¹H NMR (300 MHz, methanol-d4) δ [ppm] 1.76-1.90 (m, 1 H) |
| | 1.90-2.05 (m, 2 H) 2.13 (dt, J = 9.38, 4.69 Hz, 1 H) 3.02 (br. s., 1 H) 3.15-3.26 (m, 2 |
| | H) 3.34-3.42 (m, 2 H) 4.68 (s, 2 H) 6.72 (d, J = 8.50 Hz, 1 H) 6.99 (d, J = 7.03 Hz, |
| | 1 H) 7.60 (t, J = 7.77 Hz, 1 H) 7.71 (d, J = 9.38 Hz, 1 H) 8.32 (s, 1 H) |
| | 8.35 (s, 2 H) 8.50 (s, 1 H) |
| 47 | ¹H NMR (400 MHz, methanol-d4) δ [ppm] 1.37 (dd, J = 12.52, 4.30 Hz, 2 H) |
| | 1.66-1.77 (m, 2 H) 1.78-1.89 (m, 1 H) 1.90-2.10 (m, 4 H) 2.10-2.25 (m, |
| | 2 H) 2.26-2.39 (m, 1 H) 3.10-3.25 (m, 1 H) 3.36-3.51 (m, 2 H) |
| | 3.67-3.80 (m, 1 H) 3.96 (dd, J = 11.15, 3.33 Hz, 2 H) 6.92 (d, J = 7.83 Hz, 2 H) |
| | 7.78 (t, J = 8.02 Hz, 1 H) 8.39 (s, 1 H) 8.45 (s, 1 H) |
| 48 | ¹H NMR (400 MHz, methanol-d4) δ [ppm] 1.31-1.43 (m, J = 12.33, 12.33, |
| | 12.13, 4.30 Hz, 2 H) 1.73 (d, J = 12.91 Hz, 2 H) 1.80-1.88 (m, 1 H) |
| | 1.91-2.09 (m, 4 H) 2.11-2.22 (m, 2 H) 2.27-2.37 (m, 1 H) 3.13-3.23 (m, 1 H) |
| | 3.38-3.47 (m, 2 H) 3.74 (t, J = 5.67 Hz, 1 H) 3.96 (dd, J = 11.35, 3.13 Hz, 2 |
| | H) 6.86-6.95 (m, 2 H) 7.74-7.79 (m, 1 H) 8.39 (s, 1 H) 8.45 (s, 1 H) |
| 49 | ¹H NMR (400 MHz, methanol-d4) δ [ppm] 1.35 (qd, J = 12.39, 4.70 Hz, 2 H) |
| | 1.65-1.79 (m, 3 H) 1.83-2.08 (m, 3 H) 2.17-2.33 (m, 2 H) 2.43 (ddd, |
| | J = 13.60, 7.92, 5.48 Hz, 1 H) 3.18-3.28 (m, 2 H) 3.44 (t, J = 10.96 Hz, 2 H) |
| | 3.76 (t, J = 7.24 Hz, 1 H) 3.96 (dd, J = 11.15, 3.33 Hz, 2 H) 6.89-6.97 (m, 2 |
| | H) 7.75-7.80 (m, 1 H) 8.38 (s, 1 H) 8.43 (s, 1 H) |
| 53 | ¹H NMR (300 MHz, methanol-d4) δ [ppm] 1.73-2.04 (m, 3 H) |
| | 2.08-2.23 (m, 1 H) 2.95-3.07 (m, 1 H) 3.08-3.19 (m, 1 H) 3.23 (d, J = 4.10 Hz, 1 H) |
| | 3.35 (d, J = 6.15 Hz, 2 H) 4.57 (s, 2 H) 6.76 (d, J = 8.79 Hz, 1 H) 6.92 (d, |
| | J = 7.33 Hz, 1 H) 7.06 (t, J = 8.79 Hz, 2 H) 7.41 (dd, J = 8.50, 5.57 Hz, 2 H) |
| | 7.61-7.72 (m, 1 H) 8.34 (s, 1 H) 8.40 (s, 1 H) |
| 54 | ¹H NMR (300 MHz, methanol-d4) δ [ppm] 1.74-1.91 (m, 2 H) |
| | 1.90-2.04 (m, 1 H) 2.05-2.20 (m, 1 H) 2.98 (dd, J = 8.06, 4.25 Hz, 1 H) 3.04-3.18 (m, |
| | 1 H) 3.24-3.34 (dMeOH, 2 H App.) 3.33-3.47 (m, 2 H) 6.77 (d, J = 8.50 Hz, |
| | 1 H) 6.97 (d, J = 7.33 Hz, 1 H) 7.60 (t, J = 7.91 Hz, 1 H) 8.01 (d, J = 6.45 Hz, 2 |
| | H) 8.12 (s, 1 H) 8.28 (s, 1 H) 8.70 (d, J = 6.74 Hz, 2 H) |
| 60 | ¹H NMR (400 MHz, methanol-d4) δ [ppm] 1.18-1.47 (m, J = 12.52, 12.52, |
| | 12.13, 4.30 Hz, 3 H) 1.74 (d, J = 12.52 Hz, 2 H) 1.91-2.06 (m, 1 H) |
| | 2.62-2.73 (m, 2 H) 2.81-2.87 (m, 3 H) 3.39-3.49 (m, 4 H) 3.62-3.67 (m, 1 H) |
| | 3.72 (t, J = 9.59 Hz, 1 H) 3.96 (dd, J = 11.35, 3.52 Hz, 2 H) 6.97 (d, J = 7.04 Hz, |
| | 1 H) 7.02 (d, J = 9.00 Hz, 1 H) 7.86 (dd, J = 9.00, 7.43 Hz, 1 H) 8.41 (s, 1 H) |
| | 8.47 (s, 1 H) |
| 74 | ¹H NMR (400 MHz, methanol-d4) δ [ppm] 1.31-1.47 (m, 2 H) |
| | 1.64-1.75 (m, 2 H) 1.76-1.90 (m, 1 H) 1.90-2.08 (m, 3 H) 2.10-2.20 (m, 1 H) |
| | 2.97-3.06 (m, 1 H) 3.10-3.19 (m, 1 H) 3.19-3.27 (m, 1 H) 3.32-3.48 (m, |
| | dMeOH, 6 H App.) 3.90-4.01 (m, 2 H) 6.98 (d, J = 8.22 Hz, 1 H) 7.61 (d, |
| | J = 7.83 Hz, 1 H) 8.37 (s, 1 H) 8.52 (s, 1 H) |
| 75 | ¹H NMR (400 MHz, methanol-d4) δ [ppm] 1.30 (qd, J = 12.26, 4.30 Hz, 2 H) |
| | 1.67 (d, J = 12.91 Hz, 2 H) 1.77-2.03 (m, 4 H) 2.08-2.20 (m, 1 H) |
| | 2.98-3.07 (m, 1 H) 3.09-3.26 (m, 4 H) 3.33-3.44 (m, dMeOH, 4 App.) 3.93 (dd, |
| | J = 11.15, 3.33 Hz, 2 H) 6.58 (d, J = 9.00 Hz, 1 H) 7.48 (d, J = 9.00 Hz, 1 H) |
| | 8.17 (s, 1 H) 8.38 (s, 1 H) |
| 78 | ¹H NMR (300 MHz, methanol-d4) δ [ppm] 1.22-1.41 (m, 1 H) |
| | 1.53-1.80 (m, 3 H) 1.79-1.95 (m, 3 H) 1.98-2.15 (m, 2 H) 2.88 (td, J = 8.06, 3.52 Hz, |
| | 1 H) 2.92-3.03 (m, 1 H) 3.04-3.18 (m, 2 H) 3.18-3.28 (m, 4 H) |
| | 3.36-3.49 (m, 1 H) 3.75-3.88 (m, 1 H) 3.96 (dd, J = 10.99, 2.78 Hz, 1 H) 6.55 (d, |
| | J = 8.50 Hz, 1 H) 6.89 (d, J = 7.03 Hz, 1 H) 7.38-7.54 (m, 1 H) 8.34 (s, 1 H) |
| | 8.44 (s, 1 H) |
| 79 | ¹H NMR (300 MHz, methanol-d4) δ [ppm] 1.22-1.43 (m, 1 H) |
| | 1.52-1.81 (m, 3 H) 1.81-1.98 (m, 3 H) 1.97-2.17 (m, 2 H) 2.83-2.93 (m, 1 H) |
| | 2.93-3.04 (m, 1 H) 3.05-3.20 (m, 2 H) 3.20-3.28 (m, 4 H) 3.43 (td, J = 10.48, |
| | 3.66 Hz, 1 H) 3.77-3.88 (m, 1 H) 3.97 (dd, J = 10.99, 2.49 Hz, 1 H) 6.55 (d, |
| | J = 8.50 Hz, 1 H) 6.89 (d, J = 7.33 Hz, 1 H) 7.49 (t, J = 7.91 Hz, 1 H) 8.35 (s, 1 |
| | H) 8.41 (s, 1 H) |
| 80 | ¹H NMR (300 MHz, methanol-d4) δ [ppm] 1.73-2.06 (m, 3 H) |
| | 2.06-2.22 (m, 1 H) 2.96-3.07 (m, 1 H) 3.08-3.18 (m, 1 H) 3.18-3.25 (m, 1 H) |
| | 3.35 (d, J = 5.86 Hz, 2 H) 3.61-3.79 (m, 2 H) 4.60 (s, 2 H) 6.69 (d, J = 8.50 Hz, 1 |
| | H) 6.72-6.84 (m, 1 H) 6.91-7.03 (m, 3 H) 7.61 (t, J = 7.91 Hz, 1 H) 8.32 (s, |
| | 1 H) 8.36 (s, 1 H) |
| 81 | ¹H NMR (300 MHz, methanol-d4) δ [ppm] 1.30-1.48 (m, 1 H) |
| | 1.54-1.78 (m, 4 H) 1.83-2.13 (m, 4 H) 2.72-2.83 (m, 2 H) 2.85 (s, 3 H) 2.96 (t, |
| | J = 11.14 Hz, 1 H) 3.27-3.33 (dMeOH, 1 H App.) 3.34 (d, J = 3.81 Hz, 2 H) |
| | 3.42-3.58 (m, 1 H) 3.67 (d, J = 11.72 Hz, 1 H) 3.74-3.87 (m, 2 H) |
| | 3.87-3.97 (m, 1 H) 6.90-7.07 (m, 2 H) 7.86 (dd, J = 9.08, 7.33 Hz, 1 H) 8.37 (s, 1 |
| | H) 8.46 (s, 1 H) |
| 87 | ¹H NMR (400 MHz, methanol-d4) δ [ppm] 1.29 (m, 2 H) 1.62-1.71 (m, 2 |
| | H) 1.80-1.97 (m, 1 H) 3.14-3.20 (m, 2 H) 3.21-3.30 (m, 2 H) |
| | 3.32-3.44 (m, 3 H) 3.62-3.67 (m, 1 H) 3.87-4.01 (m, 3 H) 4.25-4.30 (m, 1 H) |
| | 4.49-4.54 (m, 1 H) 6.56-6.61 (m, 1 H) 7.44-7.51 (m, 1 H) 8.15 (s, 1 H) 8.41 (s, |
| | 1 H) |
| 88 | ¹H NMR (400 MHz, methanol-d4) δ [ppm] 1.29 (m, 2 H) 1.61-1.70 (m, 2 |
| | H) 1.80-1.96 (m, 1 H) 2.20-2.34 (m, 1 H) 2.34-2.47 (m, 1 H) |
| | 3.14-3.22 (m, 2 H) 3.35-3.48 (m, 6 H) 3.62-3.69 (m, 1 H) 3.89-3.95 (m, 2 H) |
| | 6.55-6.60 (m, 1 H) 7.44-7.53 (m, 1 H) 8.18 (s, 1 H) 8.40 (s, 1 H) |
| 91 | ¹H NMR (400 MHz, methanol-d4) δ [ppm] 1.31-1.43 (m, J = 12.42, 12.42, |
| | 12.13, 4.50 Hz, 2 H) 1.60-1.79 (m, 4 H) 1.85-2.12 (m, 3 H) 2.74-2.82 (m, |
| | 2 H) 2.85 (s, 3 H) 2.96 (t, J = 11.15 Hz, 1 H) 3.33 (br. s., 2 H) 3.44 (t, J = 11.15 Hz, |
| | 2 H) 3.67 (d, J = 11.35 Hz, 1 H) 3.79-3.86 (m, 1 H) 3.97 (dd, J = 11.35, |
| | 3.52 Hz, 2 H) 6.96 (d, J = 7.43 Hz, 1 H) 7.04 (d, J = 9.00 Hz, 1 H) 7.88 (t, |
| | J = 8.02 Hz, 1 H) 8.38 (s, 1 H) 8.47 (s, 1 H) |
| 92 | ¹H NMR (400 MHz, methanol-d4) δ [ppm] 1.26-1.36 (m, 4 H) |
| | 1.36-1.43 (m, 1 H) 1.59-1.79 (m, 4 H) 1.88 (dt, J = 6.26, 3.13 Hz, 1 H) 1.98 (dddd, |
| | J = 18.59, 11.05, 7.14, 3.13 Hz, 1 H) 2.07 (d, J = 9.00 Hz, 1 H) 2.68-2.80 (m, 1 |
| | H) 2.82-2.94 (m, 1 H) 2.98-3.11 (m, 3 H) 3.44 (t, J = 11.15 Hz, 2 H) |
| | 3.63-3.74 (m, 1 H) 3.74-3.79 (m, 1 H) 3.84 (dt, J = 12.13, 1.76 Hz, 2 H) 3.97 (dd, |
| | J = 11.35, 3.52 Hz, 2 H) 6.96 (d, J = 7.43 Hz, 1 H) 7.04 (s, 1 H) 7.86 (s, 1 H) |
| | 8.37 (s, 1 H) 8.47 (s, 1 H) |
| 96 | ¹H NMR (300 MHz, DMSO-d6) δ [ppm] 1.08-1.26 (m, J = 12.31, 12.31, |
| | 12.01, 4.40 Hz, 2 H) 1.61 (d, J = 12.89 Hz, 2 H) 1.79-1.94 (m, J = 10.77, 7.18, |
| | 3.77, 3.77 Hz, 1 H) 2.57-2.70 (m, 3 H) 3.01 (dd, J = 12.31, 2.64 Hz, 1 H) |
| | 3.12 (t, J = 6.15 Hz, 2 H) 3.19-3.28 (m, 2 H) 3.47-3.60 (m, 1 H) |
| | 3.77-3.91 (m, 3 H) 4.07 (dd, J = 9.96, 2.64 Hz, 1 H) 6.55 (d, J = 8.50 Hz, 1 H) 6.81 (d, |
| | J = 7.03 Hz, 1 H) 6.87 (t, J = 5.57 Hz, 1 H) 7.47 (t, J = 7.91 Hz, 1 H) 8.32 (s, 1 |
| | H) 8.43 (s, 1 H) 9.67 (s, 1 H) |
| 99 | ¹H NMR (400 MHz, methanol-d4) δ [ppm] 1.28-1.35 (m, 2 H) |
| | 1.64-1.71 (m, 2 H) 1.80-1.91 (m, 2 H) 1.99-2.09 (m, 2 H) 2.13-2.22 (m, 2 H) |
| | 2.26-2.34 (m, 1 H) 3.18-3.22 (m, 3 H) 3.34-3.42 (m, 2 H) 3.72-3.76 (m, 1 H) |
| | 3.90-3.96 (m, 2 H) 6.57-6.60 (m, 1 H) 7.46-7.51 (m, 1 H) 8.18 (s, 1 H) |
| | 8.38 (s, 1 H) |
| 100 | ¹H NMR (400 MHz, methanol-d4) δ [ppm] 1.28-1.35 (m, 2 H) |
| | 1.64-1.71 (m, 2 H) 1.80-1.91 (m, 2 H) 1.99-2.09 (m, 2 H) 2.13-2.22 (m, 2 H) |
| | 2.26-2.34 (m, 1 H) 3.18-3.22 (m, 3 H) 3.34-3.42 (m, 2 H) 3.72-3.76 (m, 1 H) |
| | 3.90-3.96 (m, 2 H) 6.57-6.60 (m, 1 H) 7.46-7.51 (m, 1 H) 8.18 (s, 1 H) |
| | 8.38 (s, 1 H) |
| 101 | ¹H NMR (400 MHz, methanol-d4) δ [ppm] 1.29 (m, 2 H) 1.62-1.71 (m, 2 |
| | H) 1.80-1.97 (m, 1 H) 3.14-3.20 (m, 2 H) 3.21-3.30 (m, 2 H) |
| | 3.32-3.44 (m, 3 H) 3.62-3.67 (m, 1 H) 3.87-4.01 (m, 3 H) 4.25-4.30 (m, 1 H) |
| | 4.49-4.54 (m, 1 H) 6.56-6.61 (m, 1 H) 7.44-7.51 (m, 1 H) 8.15 (s, 1 H) 8.41 (s, |
| | 1 H) |
| 102 | ¹H NMR (400 MHz, methanol-d4) δ [ppm] 1.15-1.48 (m, 2 H) |
| | 1.56-1.78 (m, 2 H) 1.75-2.03 (m, 1 H) 3.16-3.23 (m, 2 H) 3.34-3.44 (m, 2 H) |
| | 3.50-3.68 (m, 2 H) 3.69-3.82 (m, 2 H) 3.87-4.04 (m, 3 H) 5.48-5.85 (m, 1 H) |
| | 6.60 (d, J = 9.0 Hz, 1 H) 7.40 (d, J = 9.00 Hz, 1 H) 8.14 (s, 1 H) 8.40 (s, 1 H) |
| 105 | ¹H NMR (400 MHz, methanol-d4) δ [ppm] 1.29-1.32 (m, 2 H) |
| | 1.48-1.60 (m, 1 H) 1.63-1.82 (m, 4 H) 1.84-1.96 (m, 1 H) 1.96-2.13 (m, 2 H) |
| | 2.57-2.70 (m, 1 H) 2.82-2.95 (m, 1 H) 2.98-3.15 (m, 1 H) 3.18-3.25 (m, 2 H) |
| | 3.49 (m, 3 H) 3.61-3.77 (m, 3 H) 3.85-4.05 (m, 3 H) 4.12-4.29 (m, 1 H) |
| | 6.58-6.73 (m, 1 H) 7.49-7.64 (m, 1 H) 8.18 (s, 1 H) 8.40 (s, 1 H) |
| 106 | ¹H NMR (400 MHz, methanol-d4) δ [ppm] 1.29-1.40 (m, 2 H) |
| | 1.53-1.78 (m, 4 H) 1.81-2.06 (m, 4 H) 2.19-2.39 (m, 1 H) 2.96-3.01 (m, 1 H) |
| | 3.16-3.23 (m, 2 H) 3.36-3.43 (m, 2 H) 3.57-3.62 (m, 3 H) 3.84-4.08 (m, 3 H) |
| | 6.51-6.73 (m, 1 H) 7.47-7.60 (m, 1 H) 8.15-8.23 (m, 1 H) 8.32-8.42 (m, |
| | 1 H) |
| 108 | ¹H NMR (400 MHz, methanol-d4) δ [ppm] 1.22-1.36 (m, 2 H) |
| | 1.61-1.73 (m, 4 H) 1.84-1.97 (m, 2 H) 2.01-2.12 (m, 1 H) 2.68-2.82 (m, 2 H) |
| | 2.85 (s, 3 H) 2.88-2.97 (m, 1 H) 3.18-3.22 (m, 2 H) 3.35-3.45 (m, 2 H) |
| | 3.65-3.73 (m, 1 H) 3.81-3.88 (m, 1 H) 3.89-3.98 (m, 2 H) 6.61-6.68 (m, 1 H) |
| | 7.50-7.57 (m, 1 H) 8.16 (s, 1 H) 8.38 (s, 1 H) |
| 109 | ¹H NMR (400 MHz, methanol-d4) δ [ppm] 1.25-1.37 (m, 2 H) |
| | 1.63-1.77 (m, 3 H) 1.77-2.13 (m, 5 H) 2.25-2.37 (m, 1 H) 2.95 (s, 3 H) |
| | 2.97-3.05 (m, 1 H) 3.18-3.25 (m, 2 H) 3.36-3.47 (m, 2 H) 3.78-3.86 (m, 1 H) |
| | 3.89-3.97 (m, 2 H) 6.62-6.69 (m, 1 H) 7.48-7.58 (m, 1 H) 8.19 (s, 1 H) 8.38 (s, |
| | 1 H) |
| 110 | ¹H NMR (400 MHz, methanol-d4) δ [ppm] 1.22-1.37 (m, 2 H) |
| | 1.63-1.75 (m, 3 H) 1.77-1.94 (m, 2 H) 1.94-2.09 (m, 3 H) 2.28-2.37 (m, 1 H) |
| | 2.95 (s, 3 H) 2.97-3.05 (m, 1 H) 3.17-3.23 (m, 2 H) 3.35-3.44 (m, 2 H) |
| | 3.75-3.87 (m, 1 H) 3.89-3.97 (m, 2 H) 6.63-6.68 (m, 1 H) 7.55 (none, 1 H) |
| | 8.18 (s, 1 H) 8.38 (s, 1 H) |
| 111 | ¹H NMR (400 MHz, methanol-d4) δ [ppm] 1.26-1.32 (m, 5 H) |
| | 1.58-1.72 (m, 4 H) 1.80-1.96 (m, 2 H) 2.02-2.13 (m, 1 H) 2.67-2.79 (m, 1 H) |
| | 2.80-2.90 (m, 1 H) 3.00-3.08 (m, 3 H) 3.18-3.22 (m, 2 H) 3.35-3.46 (m, 2 H) |
| | 3.66-3.75 (m, 1 H) 3.82-3.89 (m, 1 H) 3.89-3.97 (m, 2 H) 6.63-6.70 (m, |
| | 1 H) 7.55 (m, 1 H) 8.16 (s, 1 H) 8.38 (s, 1 H) |
| 112 | ¹H NMR (400 MHz, methanol-d4) δ [ppm] 1.29 (s, 3 H) 1.31 (s, 3 H) |
| | 1.66-1.77 (m, 4 H) 1.77-2.00 (m, 3 H) 2.07 (m, 2 H) 2.71 (m, 1 H) |
| | 2.88-2.98 (m, 1 H) 3.06-3.17 (m, 1 H) 3.20 (d, J = 6.65 Hz, 2 H) 3.41-3.49 (m, 3 H) |
| | 3.73 (m, 1 H) 3.83-4.01 (m, 3 H) 6.65 (d, J = 9.00 Hz, 1 H) 7.54 (d, J = 9.00 Hz, |
| | 1 H) 8.15 (s, 1 H) 8.38 (s, 1 H) |
| 113 | ¹H NMR (400 MHz, methanol-d4) δ [ppm] 1.29 (m, 5 H) 1.62-1.72 (m, 3 |
| | H) 1.76-1.86 (m, 1 H) 1.87-2.09 (m, 4 H) 2.22-2.36 (m, 1 H) |
| | 2.93-3.09 (m, 3 H) 3.16-3.24 (m, 2 H) 3.34-3.45 (m, 2 H) 3.73-3.83 (m, 1 H) |
| | 3.88-3.98 (m, 2 H) 6.64-6.69 (m, 1 H) 7.52-7.57 (m, 1 H) 8.19 (s, 1 H) 8.38 (s, |
| | 1 H) |
| 115 | ¹H NMR (400 MHz, DMSO-d6) δ [ppm] 0.92-1.06 (m, 2 H) 1.10 (s, 6 H) |
| | 1.31-1.44 (m, 1 H) 1.52-1.64 (m, 4 H) 1.76-1.89 (m, 1 H) 1.93-2.09 (m, |
| | 1 H) 2.41-2.62 (m, dMeOH, 2 H App.) 2.62-2.72 (m, 1 H) 2.75-2.86 (m, |
| | 1 H) 2.92-3.01 (m, 1 H) 3.01-3.15 (m, 2 H) 3.45-3.63 (m, 2 H) 6.54 (d, |
| | J = 8.61 Hz, 1 H) 6.77 (d, J = 7.04 Hz, 1 H) 6.81 (t, J = 5.67 Hz, 1 H) |
| | 7.40-7.51 (m, 1 H) 8.33 (s, 1 H) 8.39 (s, 1 H) 10.85 (s, 1 H) |
| 116 | ¹H NMR (400 MHz, DMSO-d6) δ [ppm] 0.92-1.07 (m, 2 H) 1.10 (s, 6 H) |
| | 1.30-1.44 (m, 1 H) 1.51-1.63 (m, 4 H) 1.78-1.88 (m, 1 H) 1.96-2.09 (m, |
| | 1 H) 2.49-2.61 (m, dMeOH, 2 H App.) 2.62-2.72 (m, 1 H) 2.74-2.83 (m, |
| | 1 H) 2.91-2.99 (m, 1 H) 3.04-3.12 (m, 2 H) 3.49-3.63 (m, 2 H) 6.54 (d, |
| | J = 8.22 Hz, 1 H) 6.77 (d, J = 7.04 Hz, 1 H) 6.81 (t, J = 5.67 Hz, 1 H) |
| | 7.42-7.50 (m, 1 H) 8.33 (s, 1 H) 8.38 (s, 1 H) 10.84 (s, 1 H) |
| 121 | ¹H NMR (400 MHz, methanol-d4) δ ppm 1.28-1.46 (m, 2 H) |
| | 1.66-1.80 (m, 2 H) 1.87-2.06 (m, 1 H) 3.34-3.60 (m, 7 H) 3.62-3.71 (m, 1 H) |
| | 3.83-4.05 (m, 3 H) 4.60-4.70 (m, 1 H) 6.86-7.07 (m, 2 H) 7.74-7.93 (m, 1 H) |
| | 8.38 (s, 1 H) 8.46 (s, 1 H) |
| 122 | ¹H NMR (400 MHz, methanol-d4) δ [ppm] 1.28-1.45 (m, 2 H) |
| | 1.66-1.79 (m, 2 H) 1.88-2.04 (m, 1 H) 3.25-3.40 (m, 2 H) 3.40-3.50 (m, 5 H) |
| | 3.57-3.72 (m, 2 H) 3.90-4.03 (m, 2 H) 4.60-4.70 (m, 1 H) 6.88-6.98 (m, 2 H) |
| | 7.74-7.86 (m, 1 H) 8.31-8.41 (m, 1 H) 8.37 (s, 1 H) 8.46 (s, 1 H) |
| 123 | ¹H NMR (400 MHz, methanol-d4) δ [ppm] 1.24-1.39 (m, 2 H) 1.67 (d, |
| | J = 12.91 Hz, 2 H) 1.77-2.03 (m, 4 H) 2.08-2.20 (m, 1 H) 2.97-3.06 (m, 1 |
| | H) 3.09-3.18 (m, 1 H) 3.20-3.24 (m, 2 H) 3.31-3.46 (m, dMeOH, 5 H |
| | App.) 3.90-3.98 (m, 2 H) 6.58 (d, J = 9.00 Hz, 1 H) 7.47 (d, J = 9.00 Hz, 1 H) |
| | 8.24 (d, J = 5.09 Hz, 1 H) 8.26 (s, 1 H) |
| 124 | ¹H NMR (400 MHz, methanol-d4) δ [ppm] 1.23-1.37 (m, 2 H) 1.67 (dd, |
| | J = 13.30, 1.57 Hz, 2 H) 1.82-1.97 (m, J = 11.05, 7.34, 3.72, 3.72 Hz, 1 H) |
| | 3.21 (d, J = 7.04 Hz, 2 H) 3.24-3.45 (m, dMeOH, 6 H App.) 3.61-3.69 (m, 1 |
| | H) 3.89-3.96 (m, 2 H) 4.24-4.32 (m, 1 H) 4.51 (dd, J = 10.37, 2.93 Hz, 1 H) |
| | 6.58 (d, J = 9.00 Hz, 1 H) 7.48 (d, J = 9.00 Hz, 1 H) 8.23 (d, J = 5.09 Hz, 1 H) |
| | 8.29 (s, 1 H) |
| 128 | ¹H NMR (400 MHz, methanol-d4) δ [ppm] 0.94 (d, J = 6.65 Hz, 3 H) |
| | 1.25-1.38 (m, 3 H) 1.67-1.75 (m, 3 H) 1.93-2.09 (m, 2 H) 2.24 (t, J = 12.13 Hz, 1 |
| | H) 2.67-2.75 (m, 2 H) 3.00-3.07 (m, 1 H) 3.19-3.27 (m, 3 H) |
| | 3.39-3.49 (m, 2 H) 3.95 (dd, J = 11.35, 3.13 Hz, 2 H) 6.55 (d, J = 8.22 Hz, 1 H) 6.86 (d, |
| | J = 7.04 Hz, 1 H) 7.44-7.53 (m, 1 H) 8.33 (s, 1 H) 8.38 (s, 1 H) |
| 134 | ¹H NMR (400 MHz, methanol-d4) δ [ppm] 1.22-1.38 (m, 2 H) |
| | 1.56-1.65 (m, 2 H) 1.76-1.88 (m, 1 H) 1.88-2.03 (m, 3 H) 2.08-2.20 (m, 1 H) |
| | 2.97-3.07 (m, 1 H) 3.09-3.17 (m, 1 H) 3.18-3.24 (m, 1 H) 3.27-3.41 (m, 6 H) |
| | 3.87-3.96 (m, 2 H) 7.71 (s, 1 H) 8.19 (s, 1 H) 8.39 (s, 1 H) |
| 135 | ¹H NMR (300 MHz, DMSO-d6) δ [ppm] 1.07-1.27 (m, J = 12.31, 12.31, |
| | 12.01, 4.40 Hz, 2 H) 1.29-1.46 (m, 1 H) 1.51-1.64 (m, 4 H) 1.84 (d, |
| | J = 7.91 Hz, 1 H) 1.91-2.04 (m, 1 H) 2.53-2.62 (m, 2 H) 2.63-2.72 (m, 1 |
| | H) 2.76-2.86 (m, 1 H) 2.97 (d, J = 9.67 Hz, 1 H) 3.17-3.29 (m, 4 H) |
| | 3.81 (dd, J = 11.14, 2.64 Hz, 2 H) 6.87 (dd, J = 8.06, 2.78 Hz, 1 H) 7.00 (t, J = 4.98 Hz, |
| | 1 H) 7.38-7.50 (m, 1 H) 8.39 (s, 2 H) 10.85 (s, 1 H) |
| 136 | ¹H NMR (400 MHz, methanol-d4) δ [ppm] 1.21-1.29 (m, 3 H) |
| | 1.32-1.52 (m, 2 H) 1.62-2.04 (m, 6 H) 2.06-2.21 (m, 1 H) 2.95-3.28 (m, 3 H) |
| | 3.33-3.49 (m, 4 H) 3.81-3.92 (m, 1 H) 3.92-4.06 (m, 2 H) 6.83-7.03 (m, 2 H) |
| | 7.70-7.88 (m, 1 H) 8.38 (s, 1 H) 8.45 (s, 1 H) |
| 138 | ¹H NMR (400 MHz, methanol-d4) δ [ppm] 1.23-1.44 (m, 2 H) |
| | 1.67-1.79 (m, 2 H) 1.92-2.09 (m, 1 H) 2.90-3.16 (m, 4 H) 3.25 (d, J = 6.65 Hz, 2 H) |
| | 3.26-3.33 (m, 1 H) 3.35 (s, 3 H) 3.39-3.49 (m, 2 H) 3.89-4.01 (m, 2 H) |
| | 4.11-4.19 (m, 1 H) 6.56 (d, J = 8.0 Hz 1 H) 6.88 (d, J = 8.0 Hz 1 H) |
| | 7.43-7.54 (m, 1 H) 8.34 (s, 1 H) 8.41 (s, 1 H) |
| 141 | ¹H NMR (400 MHz, methanol-d4) δ [ppm] 1.13 (d, J = 6.65 Hz, 3 H) |
| | 1.26-1.45 (m, 4 H) 1.57-1.67 (m, 1 H) 1.73 (d, J = 12.91 Hz, 2 H) 1.82-1.93 (m, |
| | J = 13.40, 13.40, 4.70, 4.50 Hz, 1 H) 1.94-2.05 (m, 1 H) 2.05-2.12 (m, 1 H) |
| | 2.58 (d, J = 2.35 Hz, 1 H) 2.74 (ddd, J = 9.88, 6.55, 3.13 Hz, 1 H) 2.87 (dd, |
| | J = 12.91, 3.52 Hz, 1 H) 3.26 (d, J = 6.65 Hz, 2 H) 3.43 (td, J = 11.64, 1.76 Hz, 2 |
| | H) 3.95 (dd, J = 11.35, 3.52 Hz, 2 H) 6.54 (s, 1 H) 6.8 (d, J = 7.43 Hz, 1 H) |
| | 7.46-7.51 (m, 1 H) 8.32 (s, 1 H) 8.43 (s, 1 H) |
| 143 | ¹H NMR (400 MHz, methanol-d4) δ [ppm] 1.27-1.43 (m, 2 H) 1.70 (d, |
| | J = 12.13 Hz, 2 H) 2.03 (td, J = 7.34, 3.72 Hz, 1 H) 2.21-2.33 (m, 1 H) |
| | 2.35-2.48 (m, 1 H) 3.32-3.49 (m, dMeOH, 8 H App.) 3.60-3.69 (m, 1 H) |
| | 3.91-4.00 (m, 2 H) 6.93 (dd, J = 7.83, 3.13 Hz, 1 H) 7.30 (dd, J = 11.15, 8.02 Hz, 1 |
| | H) 8.35 (s, 1 H) 8.47 (s, 1 H) |
| 146 | ¹H NMR (300 MHz, DMSO-d6) δ [ppm] 0.99 (d, 3 H) 1.03-1.26 (m, 3 H) |
| | 1.50 (dd, J = 12.01, 2.93 Hz, 1 H) 1.61 (d, J = 10.55 Hz, 3 H) 1.81-1.94 (m, 2 |
| | H) 2.52-2.69 (m, 3 H) 3.06-3.15 (m, 3 H) 3.22-3.28 (m, 2 H) 3.82 (dd, |
| | J = 11.14, 2.93 Hz, 2 H) 6.54 (d, J = 8.50 Hz, 1 H) 6.78 (d, J = 7.33 Hz, 1 H) |
| | 6.84 (t, J = 5.57 Hz, 1 H) 7.46 (t, J = 7.77 Hz, 1 H) 8.34 (s, 1 H) 8.39 (s, 1 H) |
| | 10.67 (s, 1 H) |
| 150 | ¹H NMR (400 MHz, methanol-d4) δ [ppm] 1.21-1.38 (m, 2 H) 1.66 (m, 2 |
| | H) 1.89 (m, 1 H) 2.91-3.15 (m, 5 H) 3.30 (s, 3 H) 3.92 (d, J = 14 Hz, 2 H) |
| | 4.25-3.45 (m, 5 H) 6.56 (d, J = 9.0 Hz, 1 H) 7.47 (d, J = 9.00 Hz, 1 H) 8.15 (s, |
| | 1 H) 8.36 (s, 1 H) |
| 157 | ¹H NMR (400 MHz, methanol-d4) δ [ppm] 1.23-1.43 (m, 2 H) 1.69 (d, |
| | J = 12.52 Hz, 2 H) 1.75-2.05 (m, 4 H) 2.08-2.21 (m, 1 H) 3.02 (br. s., 1 H) |
| | 3.08-3.18 (m, 1 H) 3.21 (d, J = 7.04 Hz, 3 H) 3.33-3.46 (m, 4 H) |
| | 3.86-4.01 (m, 2 H) 6.62 (dd, J = 9.19, 2.93 Hz, 1 H) 7.36 (t, J = 9.00 Hz, 1 H) 8.28 (s, 1 |
| | H) 8.39 (s, 1 H) |
| 158 | ¹H NMR (400 MHz, methanol-d4) δ [ppm] 1.18-1.40 (m, J = 12.47, 12.47, |
| | 12.23, 4.30 Hz, 2 H) 1.59-1.77 (m, 2 H) 1.82-2.01 (m, 1 H) 3.15-3.23 (m, |
| | 2 H) 3.26 (dd, J = 7.24, 3.72 Hz, 2 H) 3.33-3.46 (m, 3 H) 3.65 (dd, J = 12.91, |
| | 2.35 Hz, 1 H) 3.87-4.03 (m, 3 H) 4.28 (dt, J = 13.30, 2.93 Hz, 1 H) 4.51 (dd, |
| | J = 10.37, 2.93 Hz, 1 H) 6.62 (dd, J = 9.19, 2.93 Hz, 1 H) 7.36 (t, J = 9.20 Hz, 1 |
| | H) 8.27 (s, 1 H) 8.42 (s, 1 H) |
| 159 | ¹H NMR (400 MHz, methanol-d4) δ [ppm] 0.82-0.97 (m, 2 H) 1.18 (d, |
| | J = 6.26 Hz, 6 H) 1.31 (d, J = 16.43 Hz, 1 H) 1.76 (dd, J = 13.30, 2.74 Hz, 2 H) |
| | 1.81-1.92 (m, 2 H) 1.93-2.09 (m, 2 H) 2.10-2.21 (m, 1 H) 2.97-3.06 (m, |
| | 1 H) 3.07-3.17 (m, 1 H) 3.27 (d, J = 6.65 Hz, 2 H) 3.34-3.39 (m, 2 H) |
| | 3.45-3.61 (m, 2 H) 6.85-6.94 (m, 2 H) 7.72-7.80 (m, 1 H) 8.37 (s, 1 H) 8.44 (s, |
| | 1 H) |
| 167 | ¹H NMR (400 MHz, methanol-d4) δ [ppm] 1.26-1.42 (m, 2 H) 1.70 (d, J = 13. [symbol] |
| | 2 H) 1.77-2.06 (m, 4 H) 2.10-2.24 (m, 1 H) 3.00-3.09 (m, 1 H) 3.10-3.19 |
| | H) 3.20-3.29 (m, 3 H) 3.34-3.47 (m, 4 H) 3.95 (d, J = 11.35 Hz, 2 H) 6.56 (d, |
| | Hz, 1 H) 7.49 (d, J = 9.00 Hz, 1 H) 7.51 (dd, 1 H) 8.37 (d, J = 5.09 Hz, 1 H) 8.44 |
| | H) |
| 169 | ¹H NMR (400 MHz, methanol-d4) δ [ppm] 0.89 (q, J = 12.13 Hz, 2 H) |
| | 1.18 (d, J = 6.26 Hz, 6 H) 1.76 (dd, J = 13.50, 2.54 Hz, 2 H) 1.98-2.08 (m, 1 H) |
| | 3.25-3.29 (m, 4 H) 3.33-3.41 (m, 1 H) 3.44-3.59 (m, 2 H) 3.65 (dd, |
| | J = 12.91, 2.35 Hz, 1 H) 3.89-4.05 (m, 1 H) 4.22-4.34 (m, 1 H) 4.52 (dd, |
| | J = 10.56, 2.74 Hz, 1 H) 6.86-6.97 (m, 2 H) 7.70-7.82 (m, 1 H) 8.38 (s, 1 |
| | H) 8.48 (s, 1 H) |
| 170 | ¹H NMR (400 MHz, methanol-d4) δ [ppm] 0.90 (q, J = 12.13 Hz, 2 H) |
| | 1.18 (d, J = 6.26 Hz, 6 H) 1.77 (dd, J = 13.30, 2.74 Hz, 2 H) 2.00-2.05 (m, 1 H) |
| | 2.28 (m, 1 H), 2.36-2.48 (m, 1 H) 3.28 (s, 1 H), 3.32 (m, dMeOH, 1 H |
| | App.), 3.35-3.44 (m, 2 H) 3.44-3.57 (m, 4 H) 3.59-3.68 (m, 1 H) 6.93 (d, |
| | J = 7.04 Hz, 1 H) 7.02 (d, J = 9.00 Hz, 1 H) 7.86 (dd, J = 9.00, 7.43 Hz, 1 H) |
| | 8.37 (s, 1 H) 8.47 (s, 1 H) |
| 171 | ¹H NMR (400 MHz, methanol-d4) δ [ppm] 0.90 (q, J = 12.13 Hz, 2 H) |
| | 1.18 (d, J = 5.87 Hz, 6 H) 1.76 (dd, J = 13.50, 2.54 Hz, 2 H) 1.95-2.09 (m, 1 H) |
| | 3.27 (s, 1 H) 3.42 (d, J = 3.52 Hz, 1 H) 3.45 (s, 3 H) 3.47-3.55 (m, 4 H) |
| | 3.59 (br.s, 2 H) 3.67 (d, J = 9.39 Hz, 1 H) 4.30 (d, J = 3.52 Hz, 1 H) 6.90 (d, J = 7.04 Hz, |
| | 1H) 6.97 (d, J = 8.61 Hz, 1 H) 7.82 (dd, J = 8.80, 7.24 Hz, 1 H) 8.35 (s, 1 |
| | H) 8.47 (s, 1 H) |
| 178 | ¹H NMR (400 MHz, methanol-d4) δ [ppm] 1.26-1.43 (m, 2 H) 1.73 (d, |
| | J = 11.35 Hz, 2 H) 1.91-2.01 (m, 1 H) 2.01-2.11 (m, 1 H) 2.54-2.65 (m, 1 |
| | H) 3.24-3.30 (m, dMeOH, 1 to 2 H App.) 3.37-3.49 (m, 2 H) |
| | 3.49-3.63 (m, 2 H) 3.66-3.74 (m, 1 H) 3.96 (dd, J = 11.35, 3.13 Hz, 2 H) |
| | 4.00-4.14 (m, 1 H) 4.68-4.76 (m, 1 H) 4.60-4.85 (m, dH₂O, 2 H, 1 to 2 H App.) |
| | 6.83 (d, J = 9.00 Hz, 1 H) 6.91 (d, J = 7.04 Hz, 1 H) 7.71 (t, J = 8.02 Hz, 1 H) 8.41 (s, |
| | 1 H) 8.43 (s, 1 H) |
| 180 | ¹H NMR (400 MHz, methanol-d4) δ [ppm] 1.17-1.33 (m, 2 H) |
| | 1.58-1.69 (m, 2H) 1.81-1.94 (m, 1 H) 3.09-3.19 (m, 2 H) 3.23-3.30 (m, 1 H) |
| | 3.29-3.39 (m, 2 H) 3.51-3.60 (m, 1 H) 3.81-3.93 (m, 3 H) 4.14-4.23 (m, 1 H) |
| | 4.39-4.48 (m, 1 H) 6.79-6.88 (m, 2 H) 7.63-7.74 (m, 1 H) 8.29 (s, 1 H) |
| | 8.39 (s, 1 H) |
| 181 | ¹H NMR (400 MHz, methanol-d4) δ [ppm] 1.20-1.36 (m, 2 H) |
| | 1.56-1.71 (m, 2 H) 1.87-2.04 (m, 1 H) 2.90-2.98 (m, 1 H) 2.98-3.15 (m, 4 H) |
| | 3.26-3.44 (m, 7 H) 3.85-3.99 (m, 2 H) 4.10-4.22 (m, 1 H) 7.46 (s, 1 H) 8.16 (s, |
| | 1 H) 8.37 (s, 1 H) |
| 182 | ¹H NMR (400 MHz, methanol-d4) δ [ppm] 1.32 (q, J = 11.87 Hz, 2 H) |
| | 1.73 (d, J = 12.91 Hz, 3 H) 2.01 (d, J = 3.91 Hz, 1 H) 2.21 (d, J = 12.91 Hz, 1 H) |
| | 2.41 (dd, J = 7.83, 3.91 Hz, 1 H) 2.53 (td, J = 11.84, 2.93 Hz, 1 H) 2.60-2.73 (m, 2 |
| | H) 3.21-3.28 (m, 4 H) 3.45 (t, J = 11.54 Hz, 2 H) 3.95 (d, J = 10.96 Hz, 2 H) |
| | 6.55 (dd, J = 8.22, 3.52 Hz, 1 H) 6.83-6.93 (m, 1 H) 7.49 (dt, J = 11.05, 4.06 Hz, |
| | 1 H) 8.34 (d, J = 3.91 Hz, 1 H) 8.40 (d, J = 2.35 Hz, 1 H) |
| 189 | ¹H NMR (400 MHz, methanol-d4) δ [ppm] 1.05 (d, J = 6.26 Hz, 3 H) |
| | 1.12-1.30 (m, 3 H) 1.56-1.67 (m, 3 H) 1.74 (dd, J = 13.30, 3.13 Hz, 1 H) |
| | 1.91-2.09 (m, 2 H) 2.47-2.56 (m, 1 H) 2.63 (ddd, J = 11.05, 6.36, 2.54 Hz, 1 H) |
| | 2.76 (t, J = 11.74 Hz, 1 H) 3.13 (dd, J = 12.13, 1.57 Hz, 1 H) 3.26 (d, J = 6.65 Hz, |
| | 2 H) 3.31-3.41 (m, J = 10.96, 10.96 Hz, 2 H) 3.85 (dd, J = 10.96, 3.13 Hz, |
| | 2 H) 6.85 (dd, J = 7.83, 3.13 Hz, 1 H) 7.19 (dd, J = 10.96, 7.83 Hz, 1 H) |
| | 8.23 (s, 1 H) 8.34 (s, 1 H) |
| 191 | ¹H NMR (400 MHz, chloroform-d) δ [ppm] 1.35 (m, 4 H) 1.64-1.73 (m, 4 |
| | H) 1.74-1.99 (m, 2 H) 2.67-2.83 (m, 1 H) 2.91-3.03 (m, 1 H) |
| | 3.04-3.24 (m, 3 H) 3.31-3.48 (m, 2 H) 3.74-3.86 (m, 1 H) 3.86-3.95 (m, 1 H) |
| | 3.95-4.09 (m, 2 H) 4.09-4.19 (m, 1 H) 4.72 (br. s, 1 H) 6.34-6.46 (m, 2 H) |
| | 7.45-7.51 (m, 2 H) 8.28 (s, 1 H) 8.34 (s, 1 H) 10.60-10.81 (br. s, 1 H) |
| 192 | ¹H NMR (400 MHz, methanol-d4) δ [ppm] 1.53-1.72 (m, 3 H) |
| | 1.70-1.88 (m, 3 H) 1.88-2.02 (m, 2 H) 2.07-2.23 (m, 1 H) 2.98-3.08 (m, 1H) |
| | 3.10-3.24 (m, 3 H) 3.24 (s, 3H) 3.48 (s, 2 H) 3.67 (dd, J = 8.22, 2.74 Hz, 4 H) |
| | 6.67 (d, J = 9.00 Hz, 1 H) 7.48 (d, J = 9.00 Hz, 1 H) 8.13-8.21 (m, 1 H) 8.38 (s, 1 |
| | H) |
| 194 | ¹H NMR (400 MHz, chloroform-d) δ [ppm] 1.20-1.46 (m, 3 H) |
| | 1.63-1.81 (m, 2H) 1.81-2.03 (m, 5 H) 2.06-2.23 (m, 1 H) 3.3-3.3 (m, 5 H) |
| | 3.34-3.56 (m, 4 H) 3.99 (dd, J = 11.35, 3.52 Hz, 2 H) 6.65 (d, J = 9.39 Hz, 1 H) |
| | 7.68 (d, J = 9.39 Hz, 1 H) 8.22 (br. s., 1 H) 8.29 (s, 1 H) 9.11-9.35 (br. s, 1 H) |
| | 9.49 (br. s., 1 H) 9.76-9.96 (br. s, 1 H) |
| 201 | ¹H NMR (400 MHz, chloroform-d) δ [ppm] 1.36-1.45 (m, 4H) |
| | 1.70-1.73 (m, 4H) 1.81-2.0 (m, 1H) 2.71-2.81 (m, 1 H) 2.92-3.04 (m, 1 H) |
| | 3.10 (dd, J = 13.69, 4.30 Hz, 1 H) 3.20 (d, J = 13.30 Hz, 1H) 3.36-3.49 (m, 3 H) |
| | 3.82 (dd, J = 9.78, 4.30 Hz, 1 H) 3.86-3.94 (m, 1 H) 3.95-4.08 (m, 2 H) |
| | 4.09-4.25 (m, 1 H) 4.75-4.92 (br. s, 1 H) 6.92 (dd, J = 8.02, 2.93 Hz, 1 H) |
| | 7.20 (dd, J = 10.76, 8.02 Hz, 1 H) 8.31 (s, 1 H) 8.49 (s, 1 H) 10.64 (br. s, 1 H) |
| 203 | ¹H NMR (400 MHz, chloroform-d) δ [ppm] 0.82-1.02 (m, 2 H) |
| | 1.15-1.32 (m, 6 H) 1.74-1.83 (m, 2 H) 1.96 (br. s, 1 H) 2.78 (d, J = 3.91 Hz, 1H) |
| | 2.99 (td, J = 9.19, 4.70 Hz, 1 H) 3.05-3.28 (m, 3 H) 3.35-3.58 (m, 3 H) |
| | 3.70-3.97 (m, 2 H) 3.97-4.23 (m, 2 H) 4.67-4.88 (m, 1 H) 6.41 (d, J = 8.22 Hz, 1 |
| | H) 6.92 (d, J = 7.43 Hz, 1 H) 7.45-7.58 (m, 1 H) 8.31 (s, 1 H) 8.45 (s, 1 H) |
| | 10.61 (br. s., 1 H) |
| 205 | ¹H NMR (400 MHz, methanol-d4) δ [ppm] 1.16-1.32 (m, 2 H) 1.62 (d, |
| | J = 13.30 Hz, 2 H) 1.92-2.04 (m, 1 H) 2.96-3.14 (m, 3 H) 3.27 (d, J = 7.04 Hz, |
| | 2 H) 3.34 (t, J = 11.54 Hz, 2 H) 3.43 (dd, J = 12.72, 2.54 Hz, 1 H) |
| | 3.73-3.89 (m, 3 H) 4.11 (d, J = 12.13 Hz, 1 H) 4.29 (dd, J = 10.56, 2.74 Hz, 1 H) |
| | 6.88 (dd, J = 8.02, 2.93 Hz, 1 H) 7.21 (dd, J = 11.15, 8.02 Hz, 1 H) 8.28 (s, 1 |
| | H) 8.39 (s, 1 H) |
| 206 | ¹H NMR (400 MHz, methanol-d4) δ [ppm] 1.32-1.47 (m, 9 H) |
| | 1.64-1.80 (m, 3 H) 1.82-2.03 (m, 3 H) 2.06-2.23 (m, 1 H) 2.89-3.04 (m, 1 H) |
| | 3.30-3.51 (m, 6 H) 3.97 (dd, J = 10.96, 3.52 Hz, 2 H) 6.86-7.04 (m, 2 H) 7.83 (dd, |
| | J = 9.00, 7.04 Hz, 1 H) 8.39 (s, 1 H) 8.46 (s, 1 H) |
| 207 | ¹H NMR (400 MHz, methanol-d4) δ [ppm] 1.13 (s, 3 H) 1.34-1.46 (m, 2 H) |
| | 1.60-1.73 (m, 2 H) 1.77-1.89 (m, 1 H) 1.89-2.03 (m, 2 H) 2.08-2.20 (m, |
| | 1 H) 2.98-3.08 (m, 1 H) 3.15 (dd, J = 8.22, 3.52 Hz, 1 H) 3.22 (dd, J = 6.65, |
| | 3.91 Hz, 1 H) 3.33-3.37 (m, 2 H) 3.40 (s, 2 H) 3.60-3.70 (m, 2 H) |
| | 3.74-3.85 (m, 2 H) 6.97 (dd, J = 13.69, 8.22 Hz, 2 H) 7.66-7.87 (m, 1 H) 8.42 (s, 1 |
| | H) 8.45 (s, 1 H) |
| 208 | ¹H NMR (400 MHz, methanol-d4) δ [ppm] 1.07 (s, 3 H) 1.31-1.40 (m, 2 H) |
| | 1.51-1.69 (m, 3 H) 1.70-1.82 (m, 2 H) 1.95-2.07 (m, 1 H) 2.52-2.78 (m, |
| | 2 H) 2.78-3.02 (m, 2 H) 3.06-3.15 (m, 1 H) 3.52 (s, 2 H) 3.56-3.69 (m, 2 |
| | H) 3.73-3.86 (m, 2 H) 6.93 (dd, J = 8.02, 2.93 Hz, 1 H) 7.30 (dd, J = 11.15, |
| | 8.02 Hz, 1 H) 8.32 (s, 1 H) 8.43 (s, 1 H) |
| 209 | ¹H NMR (400 MHz, methanol-d4) δ [ppm] 0.91 (t, J = 7.43 Hz, 3 H) |
| | 1.40-1.49 (m, 2 H) 1.53 (q, 2 H) 1.56-1.65 (m, 2 H) 1.74-1.90 (m, 1 H) |
| | 1.90-2.05 (m, 2 H) 2.05-2.23 (m, 1 H) 2.93-3.09 (m, 1 H) 3.10-3.24 (m, 2 H) |
| | 3.27-3.35 (m, 1 H) 3.36-3.52 (m, dMeOH, 1 H App.) 3.54-3.71 (m, 4 H) |
| | 3.73-3.90 (m, J = 11.59, 7.80, 7.80, 3.72 Hz, 2 H) 6.98 (dd, J = 8.02, 2.93 Hz, |
| | 1 H) 7.31 (dd, J = 10.96, 7.83 Hz, 1 H) 8.36 (s, 1 H) 8.51 (s, 1 H) |
| 215 | ¹H NMR (400 MHz, methanol-d4) δ [ppm] 1.03-1.27 (m, 2 H) 1.20 (s, 3 H) |
| | 1.21 (s, 3 H) 1.51-1.65 (m, 1 H) 1.69 (dd, J = 13.11, 2.93 Hz, 2 H) |
| | 1.72-1.84 (m, 2 H) 1.92-2.13 (m, 1 H) 2.15-2.35 (m, 1 H) 2.58-2.77 (m, 2 H) |
| | 2.88 (dd, J = 12.52, 9.39 Hz, 1 H) 2.93-3.04 (m, 1 H) 3.07-3.19 (m, 1 H) |
| | 3.23-3.43 (m, dMeOH, 2 H App.) 3.65-3.84 (m, 2 H) 6.91 (dd, J = 8.02, |
| | 2.93 Hz, 1 H) 7.29 (dd, J = 11.15, 8.02 Hz, 1 H) 8.33 (s, 1 H) 8.44 (s, 1 H) |
| 216 | ¹H NMR (400 MHz, methanol-d4) δ [ppm] 1.06-1.28 (m, 2 H) 1.20 (s, 3 H) |
| | 1.21 (s, 3 H) 1.53-1.65 (m, 1 H) 1.69 (dd, J = 12.91, 3.13 Hz, 2 H) |
| | 1.73-1.83 (m, 2 H) 1.97-2.09 (m, 1 H) 2.16-2.32 (m, 1 H) 2.60-2.76 (m, 2 H) |
| | 2.89 (dd, J = 12.52, 9.39 Hz, 1 H) 2.99 (d, J = 12.52 Hz, 1 H) 3.14 (dd, |
| | J = 12.13, 3.52 Hz, 1 H) 3.24-3.41 (m, dMeOH, 2 H App.) 3.66-3.80 (m, 2 |
| | H) 6.91 (dd, J = 8.02, 2.93 Hz, 1 H) 7.29 (dd, J = 11.15, 8.02 Hz, 1 H) 8.33 (s, |
| | 1 H) 8.43 (s, 1 H) |
| 217 | ¹H NMR (400 MHz, methanol-d4) δ [ppm] 1.28-1.51 (m, 2 H) 1.64 (d, |
| | J = 1.96 Hz, 1 H) 1.93 (m, 3 H) 1.80-2.42 (m, 4 H) 2.65 (s, 1 H) |
| | 3.00-3.06 (m, 1H) 3.07-3.20 (m, 1H) 3.20-3.27 (m, 1H) 3.27-3.51 (m, 5 H) |
| | 3.93-3.99 (m, 2 H) 7.05 (d, J = 7.83 Hz, 1 H) 7.83 (d, J = 8.22 Hz, 1H) 8.39 (s, 1 H) |
| | 8.51 (s, 1 H) |
| 231 | ¹H NMR (400 MHz, methanol-d4) δ [ppm] 8.30 (s, 1 H) 8.24 (s, 1 H) |
| | 7.41 (t, J = 8.0 Hz, 1 H) 6.81 (d, J = 6.4 Hz, 1 H) 6.50 (d, J = 8.4 Hz, 1 H) |
| | 3.62-3.68 (m, 1 H) 3.54-3.56 (m, 2 H) 3.48 (d, J = 11.2 Hz, 1 H) 3.40 (dd, J = 14.0, |
| | 4.0 Hz, 1 H) 3.27-3.32 (m, 2 H) 3.05 (dd, J = 13.2, 3.6 Hz, 1 H) |
| | 2.83-2.93 (m, 2 H) 2.58-2.69 (m, 2 H) 1.90-1.97 (m, 1 H) 1.65-1.74 (m, 2 H) |
| | 1.49-1.56 (m, 1 H) 1.22 (s, 3 H) 0.99 (s, 3 H) |
| 234 | ¹H NMR (400 MHz, methanol-d4) δ [ppm] 8.30 (s, 1H) 8.24 (d, J = 0.4 Hz, |
| | 1H) 7.41 (dd, J = 8.4, 7.6 Hz, 1H) 6.81 (dd, J = 7.2, 0.8 Hz, 1H) 6.49 (dd, J = 8.4, |
| | 0.8 Hz, 1H) 4.01-4.08 (m, 1H) 3.77 (dd, J = 11.6, 3.2 Hz, 1H) 3.39 (d, J = 11.2 Hz, |
| | 1H) 3.34 (dd, J = 13.6, 4.8 Hz, 1H) 3.09-3.19 (m, 1H) 3.04 (dd, J = 12.8, |
| | 3.6 Hz, 1H) 2.86-2.92 (m, 2H) 2.59-2.71 (m, 2H) 1.92-1.96 (m, 1H) |
| | 1.66-1.76 (m, 2H) 1.48-1.57 (m, 1H) 1.22 (s, 3H) 1.02 (s, 3H) |
| 232 | ¹H NMR (400 MHz, methanol-d4) δ [ppm] 8.29 (s, 1 H) 8.24 (d, J = 0.4 Hz, |
| | 1 H) 7.41 (dd, J = 8.4, 7.2 Hz, 1 H) 6.81 (dd, J = 7.2, 0.8 Hz, 1 H) 6.50 (dd, J = 8.4, |
| | 0.8 Hz, 1 H) 3.62-3.68 (m, 1 H) 3.52-3.57 (m, 2 H) 4.48 (d, J = 11.2 Hz, |
| | 1 H) 3.39 (dd, J = 14.0, 5.2 Hz, 1 H) 3.28-3.34 (m, 2 H) 3.03 (dd, J = 12.4, |
| | 3.6 Hz, 1 H) 2.87 (dt, J = 12.4, 4.0 Hz, 1 H) 2.79 (dd, J = 12.4, 9.6 Hz, |
| | 1 H) 2.52-2.63 (m, 2 H) 1.90-1.96 (m, 1 H) 1.62-1.72 (m, 2 H) |
| | 1.46-1.53 (m, 1 H) 1.22 (s, 3 H) 0.99 (s, 3 H) |
| 234 | ¹H NMR (400 MHz, methanol-d4) δ [ppm] 8.30 (s, 1H) 8.24 (d, J = 0.4 Hz, |
| | 1H) 7.41 (dd, J = 8.4, 7.6 Hz, 1H) 6.81 (dd, J = 7.2, 0.8 Hz, 1H) 6.49 (dd, J = 8.4, |
| | 0.8 Hz, 1H) 4.01-4.08 (m, 1H) 3.77 (dd, J = 11.6, 3.2 Hz, 1H) 3.39 (d, J = 11.2 Hz, |
| | 1H) 3.34 (dd, J = 13.6, 4.8 Hz, 1H) 3.09-3.19 (m, 1H) 3.04 (dd, J = 12.8, |
| | 3.6 Hz, 1H) 2.86-2.92 (m, 2H) 2.59-2.71 (m, 2H) 1.92-1.96 (m, 1H) |
| | 1.66-1.76 (m, 2H) 1.48-1.57 (m, 1H) 1.22 (s, 3H) 1.02 (s, 3H) |
| 235 | ¹H NMR (400 MHz, methanol-d4) δ [ppm] 8.38 (s, 1 H) 8.31-8.36 (m, 1 H) |
| | 7.48 (dd, J = 8.41, 7.29 Hz, 1 H) 6.86 (dd, J = 7.29, 0.68 Hz, 1 H) 6.55 (dd, |
| | J = 8.46, 0.68 Hz, 1 H) 3.94 (dd, J = 11.37, 2.67 Hz, 2 H) 3.39-3.50 (m, 3 H) |
| | 3.36 (s, 3H) 3.17-3.27 (m, 4 H) 2.74-2.89 (m, 2 H) 2.55-2.68 (m, 1 H) |
| | 1.95-2.13 (m, 2 H) 1.66-1.82 (m, 4 H) 1.24-1.39 (m, 3 H) |
| 236 | ¹H NMR (400 MHz, methanol-d4) δ [ppm] 8.38 (s, 1 H) 8.31-8.36 (m, 1 H) |
| | 7.48 (dd, J = 8.41, 7.29 Hz, 1 H) 6.86 (dd, J = 7.29, 0.68 Hz, 1 H) 6.55 (dd, |
| | J = 8.46, 0.68 Hz, 1 H) 3.94 (dd, J = 11.37, 2.67 Hz, 2 H) 3.39-3.50 (m, 3 H) |
| | 3.36 (s, 3H) 3.17-3.27 (m, 4 H) 2.74-2.89 (m, 2 H) 2.55-2.68 (m, 1 H) |
| | 1.95-2.13 (m, 2 H) 1.66-1.82 (m, 4 H) 1.24-1.39 (m, 3 H) |
| 238 | ¹H NMR (400 MHz, methanol-d4) δ [ppm] 0.87-1.07 (m, 1 H) |
| | 1.64-2.08 (m, 7 H) 2.51-2.73 (m, 2 H) 2.77-2.99 (m, 2 H) 3.08 (dd, J = 12.52, 3.52 Hz, |
| | 1 H) 3.63 (td, J = 11.54, 3.13 Hz, 2 H) 3.76 (s, 2 H) 3.91-4.05 (m, 2 H) |
| | 6.65 (d, J = 7.83 Hz, 1 H) 7.01 (d, J = 7.43 Hz, 1 H) 7.40-7.62 (m, 1 H) |
| | 8.25-8.38 (m, 1 H) 8.46 (s, 1H). |
| 239 | ¹H NMR (400 MHz, methanol-d4) δ [ppm] 1.07 (s, 3 H) 1.30-1.42 (m, 2 H) |
| | 1.56-1.71 (m, 2 H) 1.86-2.48 (m, 2 H) 3.33-3.53 (m, 9 H) 3.54-3.69 (m, |
| | 3 H) 3.72-3.87 (m, 3 H) 6.52-6.66 (m, 1 H) 6.80-6.93 (m, 1 H) |
| | 7.39-7.54 (m, 1 H) 8.34 (s, 1 H) 8.46 (s, 1 H) |
| 240 | ¹H NMR (400 MHz, methanol-d4) δ [ppm] 0.95 (t, J = 7.63 Hz, 3 H) |
| | 1.09-1.23 (m, 1 H) 1.25-1.37 (m, 2 H) 1.37-1.53 (m, J = 14.72, 14.72, 7.14, 6.85 Hz, |
| | 2 H) 1.59-1.69 (m, 1 H) 1.73 (d, J = 12.91 Hz, 2 H) 1.86 (dd, J = 13.11, |
| | 2.93 Hz, 1 H) 1.94-2.10 (m, 2 H) 2.35-2.47 (m, 1 H) 2.51-2.63 (m, 1 H) |
| | 2.79 (t, J = 11.74 Hz, 1 H) 3.19 (d, J = 10.96 Hz, 1 H) 3.24 (d, J = 6.65 Hz, 2 H) |
| | 3.38-3.52 (m, 2 H) 3.94 (dd, J = 11.15, 3.33 Hz, 2 H) 6.54 (d, J = 8.61 Hz, 1 |
| | H) 6.86 (d, J = 7.04 Hz, 1 H) 7.48 (t, J = 8.02 Hz, 1 H) 8.33 (s, 1 H) 8.38 (s, 1 |
| | H) |
| 242 | ¹H NMR (400 MHz, methanol-d4) δ [ppm] 1.05-1.16 (m, 4 H) |
| | 1.16-1.27 (m, 8 H) 1.64-1.75 (m, 3 H) 1.79 (dd, J = 13.30, 2.74 Hz, 1 H) |
| | 1.98-2.07 (m, 1 H) 2.11-2.23 (m, 1 H) 2.50-2.69 (m, 2 H) 2.80 (t, J = 11.93 Hz, 1 H) |
| | 3.14-3.28 (m, 3 H) 3.67-3.79 (m, 2 H) 6.54 (d, J = 8.22 Hz, 1 H) 6.84 (d, |
| | J = 7.04 Hz, 1 H) 7.48 (t, J = 7.83 Hz, 1 H) 8.33 (s, 1 H) 8.37 (s, 1 H) |
| 243 | ¹H NMR (400 MHz, methanol-d4) δ [ppm] 1.06-1.15 (m, 4 H) |
| | 1.15-1.28 (m, 8 H) 1.63-1.75 (m, 3 H) 1.79 (dd, J = 13.30, 2.74 Hz, 1 H) |
| | 1.99-2.07 (m, 1 H) 2.12-2.23 (m, 1 H) 2.51-2.60 (m, 1 H) 2.64 (ddd, J = 11.05, 6.36, |
| | 2.54 Hz, 1 H) 2.80 (t, J = 11.93 Hz, 1 H) 3.13-3.27 (m, 3 H) 3.67-3.79 (m, 2 |
| | H) 6.54 (d, J = 8.22 Hz, 1 H) 6.85 (d, J = 7.04 Hz, 1 H) 7.48 (t, 1 H) 8.33 (s, 1 |
| | H) 8.37 (s, 1 H) |
| 244 | ¹H NMR (400 MHz, methanol-d4) δ [ppm] 1.05-1.16 (m, 4 H) |
| | 1.16-1.33 (m, 8 H) 1.61-1.75 (m, 3 H)1.79 (dd, J = 13.30, 2.74 Hz, 1 H) 1.98-2.08 (m, |
| | 1 H) 2.10-2.25 (m, 1 H) 2.50-2.61 (m, J = 11.69, 7.87, 3.91, 3.91 Hz, 1 H) |
| | 2.61-2.71 (m, 1 H) 2.81 (t, J = 11.93 Hz, 1 H) 3.09-3.28 (m, 3 H) |
| | 3.65-3.80 (m, 2 H) 6.54 (d, J = 8.22 Hz, 1 H) 6.85 (d, J = 7.04 Hz, 1 H) 7.48 (t, |
| | J = 8.02 Hz, 1 H) 8.31 (s, 1 H) 8.38 (s, 1 H) |
| 248 | ¹H NMR (400 MHz, methanol-d4) δ [ppm] 1.78 (m, 6 H) 1.89-2.02 (m, 2 |
| | H) 2.04-2.23 (m, 1 H) 3.00-3.08 (m, 1 H) 3.14-3.23 (m, 2 H) |
| | 3.23-3.34 (m, 3 H) 3.36-3.50 (m, 1 H) 3.63-3.83 (m, 6 H) 6.99-7.07 (m, 1 H) |
| | 7.34 (dd, J = 10.96, 7.83 Hz, 1 H) 8.36 (s, 1 H) 8.53 (s, 1 H) |
| 259 | ¹H NMR (400 MHz, methanol-d4) δ [ppm] 1.09-1.29 (m, 8 H) |
| | 1.64-1.78 (m, 2 H) 2.05-2.32 (m, 2 H) 2.42 (dd, J = 13.69, 7.43 Hz, 1 H) |
| | 3.24-3.33 (m, 2 H) 3.36-3.42 (m, 2 H) 3.44-3.536 (m, 2 H) 3.63-3.67 (m, 1H) |
| | 3.69-3.78 (m, 2 H) 6.93 (d, J = 7.43 Hz, 1 H) 7.02 (d, J = 9.00 Hz, 1 H) 7.86 (t, |
| | J = 8.22 Hz, 1 H) 8.38 (s, 1 H) 8.47 (s, 1 H) |
| 260 | ¹H NMR (400 MHz, methanol-d4) δ [ppm] 1.08-1.34 (m, 8 H) |
| | 1.59-1.82 (m, 2 H) 2.05-2.33 (m, 2 H) 2.42 (dd, J = 13.30, 7.83 Hz, 1 H) |
| | 3.19-3.35 (m, 2 H) 3.33-3.42 (m, 2 H) 3.42-3.54 (m, 2 H) 3.57-3.68 (m, 1 H) |
| | 3.69-3.80 (m, 2 H) 6.93 (d, J = 7.04 Hz, 1 H) 7.02 (t, J = 8.80 Hz, 1 H) 7.85 (d, |
| | J = 7.43 Hz, 1 H) 8.38 (s, 1 H) 8.47 (s, 1 H) |
| 301 | ¹H NMR (400 MHz, methanol-d4) δ [ppm] 1.30-1.45 (m, 2 H) |
| | 1.72-1.79 (m, 2 H) 1.80-2.04 (m, 4 H) 2.10-2.20 (m, 1 H) 2.97-3.07 (m, 1 H) |
| | 3.12-3.17 (m, 3 H) 3.22-3.37 (m, dMeOH, 3 App.) 3.38-3.47 (m, 2 H) 3.97 (dd, |
| | J = 11.15, 3.72 Hz, 2 H) 7.61-7.65 (m, 1 H) 8.04 (s, 1 H) 8.09-8.11 (m, 1 H) |
| | 8.25-8.30 (m, 1 H) 8.48 (s, 1 H) |
| 310 | ¹H NMR (400 MHz, methanol-d4) δ [ppm] 1.17-1.22 (m, 6 H) |
| | 1.29-1.42 (m, 2 H) 1.74 (dd, J = 12.91, 1.96 Hz, 2 H) 1.99 (ddd, J = 11.25, 7.53, 4.30 Hz, |
| | 1 H) 2.62 (s, 3 H) 2.68-2.77 (m, 1 H) 3.26 (d, J = 7.04 Hz, 2 H) 3.41 (td, |
| | J = 11.74, 1.96 Hz, 2 H) 3.96 (dd, J = 11.35, 2.74 Hz, 2 H) 7.67 (d, J = 1.57 Hz, |
| | 1 H) 7.99 (d, J = 1.57 Hz, 1 H) 8.31 (s, 1 H) 8.47 (s, 1 H) |
| 311 | ¹H NMR (400 MHz, methanol-d4) δ [ppm] 1.30-1.43 (m, J = 12.52, 12.52, |
| | 12.13, 4.30 Hz, 2 H) 1.74 (d, J = 12.91 Hz, 2 H) 1.80-1.91 (m, 2 H) |
| | 1.93-2.05 (m, 3 H) 2.12-2.21 (m, 2 H) 2.62 (s, 3 H) 2.98-3.15 (m, 2 H) |
| | 3.22-3.28 (m, 2 H) 3.35-3.45 (m, 3 H) 3.97 (dd, J = 11.54, 3.33 Hz, 2 H) 7.62 (d, |
| | J = 1.57 Hz, 1 H) 7.97 (d, J = 1.96 Hz, 1 H) 8.30 (s, 1 H) 8.49 (s, 1 H) |
| 312 | ¹H NMR (400 MHz, methanol-d4) δ [ppm] 1.27-1.40 (m, 2 H) 1.73 (dd, |
| | J = 12.91, 1.96 Hz, 2 H) 1.78-2.02 (m, 4 H) 2.09-2.20 (m, 1 H) 3.02 (d, |
| | J = 6.65 Hz, 2 H) 3.13 (ddd, J = 12.33, 8.41, 3.52 Hz, 1 H) 3.18-3.26 (m, 1 H) |
| | 3.32-3.37 (m, 2 H) 3.37-3.46 (m, 3 H) 3.95 (dd, J = 11.35, 3.13 Hz, 2 H) |
| | 6.95 (d, J = 3.13 Hz, 1 H) 7.82 (d, J = 2.74 Hz, 1 H) 8.14 (s, 1 H) 8.42 (s, 1 H) |
| 314 | ¹H NMR (400 MHz, methanol-d4) δ [ppm] 1.27-1.40 (m, 2 H) 1.73 (dd, |
| | J = 12.91, 1.96 Hz, 2 H) 1.87 (dddd, J = 14.87, 7.63, 4.11, 3.91 Hz, 1 H) |
| | 3.02 (d, J = 6.65 Hz, 2 H) 3.23-3.29 (m, 2 H) 3.34 (br. s., 1 H) 3.35-3.46 (m, 2 |
| | H) 3.64 (dd, J = 12.91, 2.35 Hz, 1 H) 3.91-4.01 (m, 3 H) 4.27 (dt, J = 13.01, |
| | 3.08 Hz, 1 H) 4.52 (dd, J = 10.17, 3.13 Hz, 1 H) 6.96 (d, J = 3.13 Hz, 1 H) |
| | 7.83 (d, J = 3.13 Hz, 1 H) 8.13 (s, 1 H) 8.45 (s, 1 H) |
| 316 | ¹H NMR (400 MHz, chloroform-d) δ [ppm] 1.25-1.46 (m, 4H) 1.61-1.75 (m, |
| | 4H) 1.80-1.90 (m, 1 H) 2.78 (br. Ss, 1H) 2.95-3.26 (m, 4 H) 3.32-3.58 (m, |
| | 3 H) 3.74-4.21 (m, 4 H) 6.71-6.82 (m, 1 H) 7.76-7.93 (m, 1 H) 8.22 (br. |
| | s, 1 H) 8.35 (s, 1 H) |

[symbol]

indicates data missing or illegible when filed

### Biological Methods

Cdk9/cyclinT1 IMAP Protocol

The biological activity of the compounds of the invention can be determined using the assay described below.

Cdk9/cyclinT1 is purchased from Millipore, cat #14-685. The final total protein concentration in the assay is 4 nM. The 5TAMRA-cdk7tide peptide substrate, 5TAMRA-YSPTSPSYSPTSPSYSTPSPS-COOH, is purchased from Molecular Devices, cat#R7352. The final concentration of peptide substrate is 100 nM. The ATP substrate (Adenosine-5′-triphosphate) is purchased from Roche Diagnostics, cat#1140965. The final concentration of ATP substrate is 6 uM. IMAP (Immobilized Metal Assay for Phosphochemicals) Progressive Binding reagent is purchased from Molecular Devices, cat#R8139. Fluorescence polarization (FP) is used for detection. The 5TAMRA-cdk7tide peptide is phosphorylated by Cdk9/cyclinT1 kinase using the ATP substrate. The Phospho-5TAMRA-cdk7tide peptide substrate is bound to the IMAP Progressive Binding Reagent. The binding of the IMAP Progressive Binding Reagent changes the fluorescence polarization of the 5TAMRA-cdk7tide peptide which is measured at an excitation of 531 nm and FP emission of 595 nm. Assays are carried out in 100 mM Tris, pH=7.2, 10 mM MgCl₂, 0.05% NaN₃, 0.01% Tween-20, 1 mM dithiothreitol and 2.5% dimethyl sulfoxide. IMAP Progressive Binding Reagent is diluted 1:800 in 100% 1× Solution A from Molecular Devices, cat#R7285.

General protocol is as follows: To 10 uL of cdk9/cyclinT1, 0.5 uL of test compound in dimethyl sulfoxide is added. 5TAMRA-cdk7tide and ATP are mixed. 10 uL of the 5TAMRA-cdk7tide/ATP mix is added to start the reaction. The reaction is allowed to proceed for 4.5 hrs. 60 uL of IMAP Progressive Binding Reagent is added. After >1 hr of incubation, plates are read on the Envision 2101 from Perkin-Elmer. The assay is run in a 384-well format using black Corning plates, cat#3573.

Cdk9/cyclinT1 Alpha Screen Protocol

Full length wild type Cdk9/cyclin T1 is purchased from Invitogen, cat#PV4131. The final total protein concentration in the assay is 1 nM. The cdk7tide peptide substrate, biotin-GGGGYSPTSPSYSPTSPSYSPTSPS-OH, is a custom synthesis purchased from the Tufts University Core Facility. The final concentration of cdk7tide peptide substrate is 200 nM. The ATP substrate (Adenosine-5′-triphosphate) is purchased from Roche Diagnostics. The final concentration of ATP substrate is 6 uM. Phospho-Rpb1 CTD (ser2/5) substrate antibody is purchased from Cell Signaling Technology. The final concentration of antibody is 0.67 ug/mL. The Alpha Screen Protein A detection kit containing donor and acceptor beads is purchased from PerkinElmer Life Sciences. The final concentration of both donor and acceptor beads is 15 ug/mL. Alpha Screen is used for detection. The biotinylated-cdk7tide peptide is phosphorylated by cdk9/cyclinT1 using the ATP substrate. The biotinylated-cdk7tide peptide substrate is bound to the streptavidin coated donor bead. The antibody is bound to the protein A coated acceptor bead. The antibody will bind to the phosphorylated form of the biotinylated-cdk7tide peptide substrate, bringing the donor and acceptor beads into close proximity. Laser irradiation of the donor bead at 680 nm generates a flow of short-lived singlet oxygen molecules. When the donor and acceptor beads are in close proximity, the reactive oxygen generated by the irradiation of the donor beads initiates a luminescence/fluorescence cascade in the acceptor beads. This process leads to a highly amplified signal with output in the 530-620 nm range. Assays are carried out in 50 mM Hepes, pH=7.5, 10 mM MgCl₂, 0.1% Bovine Serum Albumin, 0.01% Tween-20, 1 mM Dithiolthreitol, 2.5% Dimethyl Sulfoxide. Stop and detection steps are combined using 50 mM Hepes, pH=7.5, 18 mM EDTA, 0.1% Bovine Serum Albumin, 0.01% Tween-20.

General protocol is as follows: To 5 uL of cdk9/cyclinT1, 0.25 uL of test compound in dimethyl sulfoxide is added. Cdk7tide peptide and ATP are mixed. 5 uL of the cdk7tide peptide/ATP mix is added to start the reaction. The reaction is allowed to proceed for 5 hrs. 10 uL of Ab/Alpha Screen beads/Stop-detection buffer is added. Care is taken to keep Alpha Screen beads in the dark at all times. Plates are incubated at room temperature overnight, in the dark, to allow for detection development before being read. The assay is run is a 384-well format using white polypropylene Greiner plates.

The data shown in Tables V and VI was generated using one of the assays described above.

TABLE V

| Example No. | Cdk9_cyclinT1_IC₅₀ [μM] |
|---|---|
| 1 | <0.008 |
| 2 | 0.134 |
| 3 | 0.001 |
| 4 | 0.017 |
| 5 | 0.062 |
| 6 | 0.092 |
| 7 | 0.014 |
| 8 | 0.046 |
| 9 | 0.144 |
| 10 | 0.164 |
| 11 | 0.77 |
| 12 | <0.008 |
| 13 | 0.016 |
| 14 | 0.811 |
| 15 | <0.008 |
| 16 | 0.855 |
| 17 | <0.008 |
| 18 | <0.008 |
| 19 | <0.008 |
| 20 | 0.044 |
| 21 | 0.076 |
| 22 | 0.033 |
| 23 | 0.009 |
| 24 | <0.008 |
| 25 | 0.047 |
| 26 | 0.032 |
| 27 | 0.021 |
| 28 | 0.021 |
| 29 | 0.026 |
| 30 | <0.008 |
| 31 | 0.046 |
| 32 | 0.019 |
| 33 | 0.076 |
| 34 | 0.010 |
| 35 | <0.008 |
| 36 | 0.102 |
| 37 | 0.050 |
| 38 | 0.023 |
| 39 | <0.008 |
| 40 | <0.008 |
| 41 | <0.008 |
| 42 | 0.042 |
| 43 | 0.057 |
| 44 | 0.181 |
| 45 | 0.154 |
| 46 | 0.056 |
| 47 | <0.008 |
| 48 | <0.008 |
| 49 | <0.008 |
| 50 | <0.008 |
| 51 | 0.01 |
| 52 | 0.038 |
| 53 | <0.008 |
| 54 | <0.008 |
| 55 | <0.008 |
| 56 | 0.081 |
| 57 | 0.081 |
| 58 | 0.116 |
| 59 | 0.009 |
| 60 | 0.009 |
| 61 | <0.008 |
| 62 | 0.019 |
| 63 | 0.027 |
| 64 | 0.037 |
| 65 | <0.008 |
| 66 | 0.009 |
| 67 | 0.396 |
| 68 | 0.011 |
| 69 | 0.139 |
| 70 | 0.011 |
| 71 | 0.056 |
| 72 | 0.04 |
| 73 | 0.013 |
| 74 | <0.008 |
| 75 | <0.008 |
| 76 | 0.015 |
| 77 | 0.008 |
| 78 | <0.008 |
| 79 | <0.008 |
| 80 | <0.008 |
| 81 | <0.008 |
| 82 | 0.631 |
| 83 | 0.482 |
| 84 | 0.419 |
| 85 | 0.016 |
| 86 | <0.008 |
| 87 | 0.018 |
| 88 | <0.008 |
| 89 | <0.008 |
| 90 | 0.008 |
| 91 | <0.008 |
| 92 | <0.008 |
| 93 | <0.008 |
| 94 | <0.008 |
| 95 | 0.167 |
| 96 | 0.005 |
| 97 | 0.01 |
| 98 | 0.014 |
| 99 | <0.008 |
| 100 | <0.008 |
| 101 | 0.008 |
| 102 | 0.009 |
| 103 | 0.027 |
| 104 | 0.046 |
| 105 | <0.008 |
| 106 | <0.008 |
| 107 | <0.008 |
| 108 | <0.008 |
| 109 | <0.008 |
| 110 | <0.008 |
| 111 | <0.008 |
| 112 | <0.008 |
| 113 | <0.008 |
| 114 | <0.008 |
| 115 | 0.001 |
| 116 | 0.002 |
| 117 | 0.23 |
| 118 | 0.214 |
| 119 | 0.014 |
| 120 | <0.008 |
| 121 | 0.134 |
| 122 | 0.015 |
| 123 | <0.008 |
| 124 | 0.030 |
| 125 | 0.009 |
| 126 | 0.018 |
| 127 | 0.009 |
| 128 | 0.001 |
| 129 | 0.013 |
| 130 | <0.008 |
| 131 | 0.038 |
| 132 | <0.008 |
| 133 | <0.008 |
| 134 | <0.008 |
| 135 | 0.001 |
| 136 | 0.013 |
| 137 | 0.011 |
| 138 | <0.008 |
| 139 | <0.008 |
| 140 | <0.008 |
| 141 | <0.008 |
| 142 | 0.151 |
| 143 | 0.001 |
| 144 | 0.001 |
| 145 | 0.785 |
| 146 | 0.001 |
| 147 | 0.005 |
| 148 | <0.008 |
| 149 | 0.031 |
| 150 | <0.008 |
| 151 | <0.008 |
| 152 | <0.008 |
| 153 | 0.026 |
| 154 | 0.015 |
| 155 | 0.001 |
| 156 | 0.004 |
| 157 | 0.001 |
| 158 | 0.005 |
| 159 | 0.001 |
| 160 | 0.011 |
| 161 | 0.009 |
| 162 | 0.01 |
| 163 | 0.129 |
| 164 | |
| 165 | |
| 166 | |
| 167 | 0.004 |
| 168 | 0.006 |
| 169 | 0.016 |
| 170 | 0.001 |
| 171 | 0.001 |
| 172 | 0.001 |
| 173 | 0.001 |
| 174 | 0.001 |
| 175 | 0.002 |
| 176 | 0.023 |
| 177 | 0.005 |
| 178 | 0.001 |
| 179 | 0.007 |
| 180 | 0.02 |
| 181 | 0.001 |
| 182 | 0.004 |
| 183 | 0.055 |
| 184 | 0.011 |
| 185 | 0.001 |
| 186 | 0.001 |
| 187 | 0.003 |
| 188 | 0.001 |
| 189 | 0.001 |
| 190 | 0.003 |
| 191 | 0.001 |
| 192 | 0.001 |
| 193 | 0.002 |
| 194 | 0.001 |
| 195 | 0.002 |
| 196 | 0.017 |
| 197 | 0.004 |
| 198 | 0.001 |
| 199 | 0.051 |
| 200 | 0.031 |
| 201 | 0.001 |
| 202 | 0.002 |
| 203 | 0.003 |
| 204 | 0.001 |
| 205 | 0.005 |
| 206 | 0.001 |
| 207 | 0.001 |
| 208 | 0.001 |
| 209 | 0.001 |
| 210 | 0.001 |
| 211 | 0.002 |
| 212 | 0.171 |
| 213 | 0.002 |
| 214 | 0.001 |
| 215 | 0.001 |
| 216 | 0.001 |
| 217 | 0.004 |
| 218 | 0.001 |
| 219 | 0.001 |
| 220 | 0.014 |
| 221 | 0.003 |
| 222 | 0.025 |
| 223 | 0.004 |
| 224 | 0.002 |
| 225 | 0.001 |
| 226 | 0.001 |
| 227 | 0.001 |
| 228 | 0.002 |
| 229 | 0.001 |
| 230 | 0.002 |
| 231 | 0.001 |
| 232 | 0.001 |
| 233 | 0.001 |
| 234 | 0.003 |
| 235 | 0.002 |
| 236 | 0.007 |
| 237 | 0.001 |
| 238 | 0.001 |
| 239 | 0.001 |
| 240 | 0.001 |
| 241 | 0.009 |
| 242 | 0.001 |
| 243 | 0.002 |
| 244 | 0.001 |
| 245 | 0.006 |
| 246 | 0.006 |
| 247 | 0.007 |
| 248 | 0.003 |
| 249 | 0.009 |
| 250 | 0.001 |
| 251 | 0.001 |
| 252 | 0.001 |
| 253 | 0.001 |
| 254 | 0.001 |
| 255 | 0.001 |
| 256 | 0.001 |
| 257 | 0.005 |
| 258 | 0.002 |
| 259 | 0.001 |
| 260 | 0.001 |
| 261 | 0.001 |
| 262 | 0.003 |
| 263 | 0.001 |
| 264 | 0.001 |
| 265 | 0.001 |

TABLE VI

| Example No. | Cdk9_cyclinT1_IC₅₀ [μM] |
|---|---|
| 301 | <0.008 |
| 302 | <0.008 |
| 303 | 0.025 |
| 304 | 0.081 |
| 305 | 0.376 |
| 306 | 0.046 |
| 307 | 0.239 |
| 308 | 0.531 |
| 309 | 0.627 |
| 310 | 0.147 |
| 311 | 0.103 |
| 312 | <0.008 |
| 313 | <0.008 |
| 314 | 0.003 |
| 315 | 0.004 |
| 316 | 0.006 |

1. A compound of Formula I 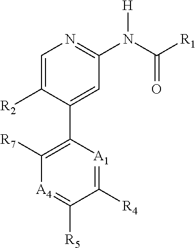
or a pharmaceutically acceptable salt thereof, wherein:
R₁ is selected from —(CH₂)₀₋₂-heteroaryl, —(CH₂)₀₋₂-aryl, C₁₋₈ alkyl, C₃₋₈ branched alkyl, C₃₋₈ cycloalkyl, and a 4 to 8 membered heterocycloalkyl group, wherein said groups are each independently optionally substituted;
R₂ is selected from hydrogen, C₁₋₄ alkoxy, C₁₋₄ haloalkyl, C₁₋₄-alkyl, and halogen;
A₁ is N;
A₄ is CR₆;
R₄ is selected from hydrogen, halogen, 5 to 7 membered heterocyclyl-R¹⁴, and A₆-L-R₉;
R₅ is selected from hydrogen, C₁₋₄ alkyl, C₁₋₄ haloalkyl, hydroxyl, CN, —O—C₁₋₄ alkyl, —O—C₁₋₄ haloalkyl, C₃₋₄ cycloalkyl, C₃₋₄ cyclo haloalkyl, and halogen;
R₆ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;
R₇ is selected from hydrogen, C₁₋₄ alkyl, C₁₋₄ haloalkyl, O—C₁₋₃ alkyl, and halogen;
A₆ is selected from O, SO₂, and NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;
R₈ is selected from hydrogen, C₁₋₄ alkyl, and C₃₋₈ branched-alkyl, and —C₃₋₈ branched haloalkyl;
R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted; and
R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl.

2. A compound of claim 1, wherein:
R₁ is selected from —(CH₂)₀₋₂-heteroaryl, —(CH₂)₀₋₂-aryl, wherein said groups are each independently optionally substituted with one to three substituents selected from —NH₂, —F, —Cl, —OH, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ cyclo haloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;
R₂ is selected from hydrogen, C₁₋₄ alkoxy, C₁₋₄ haloalkyl, C₁₋₄-alkyl, and halogen;
A₁ is N;
A₄ is CR₆;
R₄ is selected from hydrogen, halogen, 5 to 7 membered heterocyclyl-R¹⁴, and A₆-L-R₉;
R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;
R₆ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;
R₇ is selected from hydrogen, C₁₋₄ alkyl, C₁₋₄ haloalkyl, O—C₁₋₃ alkyl, and halogen;
A₆ is O, SO₂, or NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, and C₃₋₈ branched alkylene;
R₈ is selected from hydrogen, C₁₋₄ alkyl, and C₃₋₈ branched-alkyl, and —C₃₋₈ branched haloalkyl;
R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;
R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

3. A compound of claim 1, wherein:
R₁ is selected from —(CH₂)₀₋₂-heteroaryl, and —(CH₂)₀₋₂-aryl, wherein said groups are each independently optionally substituted with one to three substituents selected from the group consisting of —NH₂, F, Cl, —OH, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, —O—(CH₂)₁₋₂—C₃₋₆ cycloalkyl-R¹⁴, —O—(CH₂)₁₋₂—C₄₋₆ heterocycloalkyl-R¹⁴, —SO₂—NR¹⁵R¹⁶, and —SO₂—C₃₋₅ cycloalkyl;
R₂ is selected from hydrogen, and halogen;
A₁ is N;
A₄ is CR₆;
R₄ is selected from piperidinyl, morpholinyl, pyrrolidinyl, and A₆-L-R₉; wherein each said piperidinyl, morpholinyl, pyrrolidinyl group is substituted with R¹⁴;
R₅ is selected from hydrogen, Cl, F, and CF₃;
R₆ is hydrogen;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and C₁₋₄ alkyl;
R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl;
R¹⁴ is selected from phenyl, halogen, hydroxyl, C₁₋₂-alkyl, CF₃, and hydrogen; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

4. A compound of claim 1, wherein:
R₁ is selected from C₁₋₈ alkyl, C₃₋₈ cycloalkyl, C₃₋₈ branched alkyl, and a 4 to 8 membered heterocycloalkyl group, wherein said groups are each independently optionally substituted with one to three substituents selected from —NH₂, —F, —OH, ═O, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ halo-cycloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl; —C(O)—NR¹⁵R¹⁶, and —SO₂—NR¹⁵R¹⁶, and further wherein, any two said substituents along with the atoms to which they are attached can form a ring;
R₂ is selected from hydrogen, C₁₋₄ alkoxy, C₁₋₄ haloalkyl, C₁₋₄-alkyl, and halogen;
A₁ is N;
A₄ is CR₆;
R₄ is selected from hydrogen, halogen, 5 to 7 membered heterocyclyl-R¹⁴, and A₆-L-R₉;
R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;
R₆ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, and halogen;
R₇ is selected from hydrogen, C₁₋₄ alkyl, C₁₋₄ haloalkyl, O—C₁₋₃ alkyl, and halogen;
A₆ is selected from O, SO₂, and NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;
R₈ is selected from hydrogen, C₁₋₄ alkyl, and C₃₋₈ branched-alkyl, and —C₃₋₈ branched haloalkyl;
R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;
R¹⁴ is selected from hydrogen, phenyl, halogen, hydroxy, C₁₋₄-alkyl, C₃₋₆-branched alkyl, C₁₋₄-haloalkyl, CF₃, ═O, and O—C₁₋₄-alkyl; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

5. A compound of claim 1, wherein:
R₁ is selected from C₁₋₈ alkyl, C₃₋₈ branched alkyl, C₃₋₈ cycloalkyl, and a 4 to 8 membered heterocycloalkyl group, wherein said groups are each independently optionally substituted with one to three substituents selected from the group consisting of —NH₂, F, —OH, ═O, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;
R₂ is selected from hydrogen, and halogen;
A₁ is N;
A₄ is CR₆;
R₄ is selected from piperidinyl, morpholinyl, pyrrolidinyl, and A₆-L-R₉; wherein each said piperidinyl, morpholinyl, pyrrolidinyl group is substituted with R¹⁴;
R₅ is selected from hydrogen, Cl, F, and CF₃;
R₆ is hydrogen;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and C₁₋₄ alkyl;
R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl; and
R¹⁴ is selected from phenyl, halogen, hydroxy, C₁₋₂-alkyl, and hydrogen.

6. A compound of claim 1, wherein:
R₁ is selected from piperidinyl, morpholinyl, 1-methylpiperidinyl, tetrahydro-pyran, pyrrolidinyl, tetrahydro-furan, azetidine, pyrrolidin-2-one, azepane, and 1,4-oxazepane, wherein said R₁ groups are each independently optionally substituted with one to three substituents selected from F, OH, NH₂, CO-methyl, —NH-methyl, ethyl, fluoro-ethyl, trifluoro-ethyl, (CH₂)₂-methoxy, SO₂—CH₃, COO—CH₃, SO₂-ethyl, SO₂-cyclopropyl, methyl, SO₂—CH—(CH₃)₂, NH—SO₂—CH₃, NH—SO₂—C₂H₅, ═O, CF₃, (CH₂)-methoxy, methoxy, NH—SO₂—CH—(CH₃)₂, —(CH₂)—O—(CH₂)₂-methoxy, —O—CH—(CH₃)₂;
R₂ is selected from Cl, and F;
A₁ is N;
A₄ is CR₆;
R₄ is A₆-L-R₉;
R₅ is selected from Cl, F, and hydrogen;
R₆ is H;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and methyl; and
R₉ is selected from C₁₋₃ alkyl, C₄₋₆ branched alkyl, —(CH₂)₁₋₃—O—C₁₋₄ alkyl, —(CH₂)-pyridyl, benzyl, CD₂-tetrahydro-pyran, tetrahydro-pyran, tetrahydro-thiopyran 1,1-dioxide, piperidinyl, pyrrolidine-2-one, dioxane, cyclopropyl, tetrahydrofuran, cyclohexyl, and cycloheptyl, wherein said groups are optionally substituted with one to three substituents each independently selected from F, OCHF₂, CO-methyl, OH, methyl, methoxy, CN, ethyl, and NH—CO-methyl.

7. A compound of claim 1, wherein:
R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, and —O—CH—(CH₃)₂;
R₂ is Cl;
R₄ is A₆-L-R₉;
R₅ is selected from Cl, F, and hydrogen;
R₆ is H;
R₇ is selected from Cl, F, and hydrogen;
A₆ is NR₈;
L is selected from —CH₂—, and —CD₂-;
R₈ is selected from hydrogen, and methyl; and
R₉ is selected from pyridyl, benzyl, tetrahydro-pyran, dioxane, and tetrahydrofuran, wherein said groups are optionally substituted with one to three substituents each independently selected from F, OH, methyl, ethyl, methoxy, and CN.

8. A compound according to any one of claims 1 to 7, or pharmaceutically acceptable salt thereof, for use in a method of treating a disease or condition mediated by CDK9.

9. The use of a compound according to any one of claims 1 to 7, or a pharmaceutically acceptable salt thereof, in the manufacture of a medicament for the treatment of a disease or condition mediated by CDK9.

10. A method of treatment of a disease or condition mediated by CDK9 comprising administration to a subject in need thereof a therapeutically effective amount of a compound according to any one of claims 1 to 7, or a pharmaceutically acceptable salt thereof

11. A pharmaceutical composition comprising a compound according to any one of claims 1 to 7, or a pharmaceutically acceptable salt thereof, and a pharmaceutically acceptable carrier, diluent or excipient.

12. A compound of claim 1 selected from:
((1R,3S)-3-{3,5′-Dichloro-6-[(tetrahydro-pyran-4-ylmethyl)-amino]-[2,4′]bipyridinyl-2′-ylcarbamoyl}-cyclopentyl)-carbamic acid methyl ester;
(1S,3R)-3-(Propane-2-sulfonylamino)-cyclopentanecarboxylic acid {3,5′-dichloro-6-[(tetrahydro-pyran-4-ylmethyl)-amino]-[2,4′]bipyridinyl-2′-yl}-amide;
(S)-3-{5′-Chloro-6-[(1′,1′-dioxo-hexahydro-1-thiopyran-4-ylmethyl)-amino]-[2,4′]bipyridinyl-2′-ylcarbamoyl}-piperidine-1-carboxylic acid methyl ester;
(S)-3-{3,5′-Dichloro-6-[(2,2-dimethyl-tetrahydro-pyran-4-ylmethyl)-amino]-[2,4′]bipyridinyl-2′-ylcarbamoyl}-piperidine-1-carboxylic acid methyl ester;
((1S,3R)-3-{3,5′-Dichloro-6-[(tetrahydro-pyran-4-ylmethyl)-amino]-[2,4′]bipyridinyl-2′-ylcarbamoyl}-cyclopentyl)-carbamic acid methyl ester;
(S)-1-Methanesulfonyl-piperidine-3-carboxylic acid {3,5′-dichloro-6-[(tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[((S)-2,2-dimethyl-tetrahydro-pyran-4-ylmethyl)-amino]-[2,4′]bipyridinyl-2′-yl}-amide;
(1R,3S)-3-Methanesulfonylamino-cyclopentanecarboxylic acid {3,5′-dichloro-6-[(tetrahydro-pyran-4-ylmethyl)-amino]-[2,4′]bipyridinyl-2′-yl}-amide;
(1S,3R)-3-Ethanesulfonylamino-cyclopentanecarboxylic acid {3,5′-dichloro-6-[(tetrahydro-pyran-4-ylmethyl)-amino]-[2,4′]bipyridinyl-2′-yl}-amide;
(S)-1-Ethanesulfonyl-piperidine-3-carboxylic acid {3,5′-dichloro-6-[(tetrahydro-pyran-4-ylmethyl)-amino]-[2,4′]bipyridinyl-2′-yl}-amide;
(S)-3-{3,5′-Dichloro-6-[((R)-2,2-dimethyl-tetrahydro-pyran-4-ylmethyl)-amino]-[2,4′]bipyridinyl-2′-ylcarbamoyl}-piperidine-1-carboxylic acid methyl ester;
(S)-1-Methanesulfonyl-piperidine-3-carboxylic acid {5′-chloro-6-[(tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[(2,2-dimethyl-tetrahydro-pyran-4-ylmethyl)-amino]-[2,4′]bipyridinyl-2′-yl}-amide;
(S)-1-(Propane-2-sulfonyl)-piperidine-3-carboxylic acid {3,5′-dichloro-6-[((R)-2,2-dimethyl-tetrahydro-pyran-4-ylmethyl)-amino]-[2,4′]bipyridinyl-2′-yl}-amide;
(1S,3R)-3-Methanesulfonylamino-cyclopentanecarboxylic acid {3,5′-dichloro-6-[(tetrahydro-pyran-4-ylmethyl)-amino]-[2,4′]bipyridinyl-2′-yl}-amide;
(S)-1-Ethanesulfonyl-piperidine-3-carboxylic acid {5′-chloro-6-[(tetrahydro-pyran-4-ylmethyl)-amino]-[2,4′]bipyridinyl-2′-yl}-amide; and
(S)-1-(Propane-2-sulfonyl)-piperidine-3-carboxylic acid {3,5′-dichloro-6-[(tetrahydro-pyran-4-ylmethyl)-amino]-[2,4′]bipyridinyl-2′-yl}-amide.

13. A compound of claim 1 selected from:
(R)-Piperidine-3-carboxylic acid {5′-chloro-6-[((2R,6S)-2,6-dimethyl-tetrahydro-pyran-4-ylmethyl)-amino]-[2,4′]bipyridinyl-2′-yl}-amide;
(R)-Pyrrolidine-3-carboxylic acid {5′-chloro-6-[((S)-2,2-dimethyl-tetrahydro-pyran-4-ylmethyl)-amino]-[2,4′]bipyridinyl-2′-yl}-amide;
(R)-Pyrrolidine-3-carboxylic acid {5′-chloro-6-[((2R,6S)-2,6-dimethyl-tetrahydro-pyran-4-ylmethyl)-amino]-[2,4′]bipyridinyl-2′-yl}-amide;
(R)-Piperidine-3-carboxylic acid {5′-chloro-6-[((R)-2,2-dimethyl-tetrahydro-pyran-4-ylmethyl)-amino]-[2,4′]bipyridinyl-2′-yl}-amide;
(R)-Piperidine-3-carboxylic acid {5′-chloro-6-[((S)-6,6-dimethyl-[1,4]dioxan-2-ylmethyl)-amino]-[2,4′]bipyridinyl-2′-yl}-amide;
(R)-Piperidine-3-carboxylic acid {5′-chloro-6-[((R)-2,2-dimethyl-tetrahydro-pyran-4-ylmethyl)-amino]-5-fluoro-[2,4′]bipyridinyl-2′-yl}-amide;
(R)-Pyrrolidine-3-carboxylic acid {5′-chloro-6-[((R)-2,2-dimethyl-tetrahydro-pyran-4-ylmethyl)-amino]-[2,4′]bipyridinyl-2′-yl}-amide;
(R)-Piperidine-3-carboxylic acid {5′-chloro-6-[((S)-2,2-dimethyl-tetrahydro-pyran-4-ylmethyl)-amino]-[2,4′]bipyridinyl-2′-yl}-amide;
(R)-Piperidine-3-carboxylic acid {5′-chloro-6-[((S)-2,2-dimethyl-tetrahydro-pyran-4-ylmethyl)-amino]-5-fluoro-[2,4′]bipyridinyl-2′-yl}-amide;
(R)-Piperidine-3-carboxylic acid {5′-chloro-6-[((S)-6,6-dimethyl-[1,4]dioxan-2-ylmethyl)-amino]-5-fluoro-[2,4′]bipyridinyl-2′-yl}-amide;
(R)-Piperidine-3-carboxylic acid {5′-chloro-6-[((R)-6,6-dimethyl-[1,4]dioxan-2-ylmethyl)-amino]-[2,4′]bipyridinyl-2′-yl}-amide; and
(R)-Piperidine-3-carboxylic acid {5′-chloro-6-[((R)-5,5-dimethyl-[1,4]dioxan-2-ylmethyl)-amino]-5-fluoro-[2,4′]bipyridinyl-2′-yl}-amide.

14. A compound of claim 1 selected from:
(R)-Piperidine-3-carboxylic acid {3,5′-dichloro-6-[(tetrahydro-pyran-4-ylmethyl)-amino]-[2,4′]bipyridinyl-2′-yl}-amide;
(R)-Piperidine-3-carboxylic acid {5′-chloro-5-fluoro-6-[(tetrahydro-pyran-4-ylmethyl)-amino]-[2,4′]bipyridinyl-2′-yl}-amide;
(R)-Piperidine-3-carboxylic acid {5′-chloro-3-fluoro-6-[(tetrahydro-pyran-4-ylmethyl)-amino]-[2,4′]bipyridinyl-2′-yl}-amide;
(R)-Piperidine-3-carboxylic acid {5′-chloro-6-[(tetrahydro-pyran-4-ylmethyl)-amino]-[2,4′]bipyridinyl-2′-yl}-amide;
(R)-Pyrrolidine-3-carboxylic acid {5′-chloro-6-[(tetrahydro-pyran-4-ylmethyl)-amino]-[2,4′]bipyridinyl-2′-yl}-amide;
(R)-Pyrrolidine-3-carboxylic acid {3,5′-dichloro-6-[(tetrahydro-pyran-4-ylmethyl)-amino]-[2,4′]bipyridinyl-2′-yl}-amide;
(R)-Pyrrolidine-3-carboxylic acid {5′-chloro-5-fluoro-6-[(tetrahydro-pyran-4-ylmethyl)-amino]-[2,4′]bipyridinyl-2′-yl}-amide;
(R)-Piperidine-3-carboxylic acid {5,5′-dichloro-6-[(tetrahydro-pyran-4-ylmethyl)-amino]-[2,4′]bipyridinyl-2′-yl}-amide;
(R)-Piperidine-3-carboxylic acid {3,5,5′-trichloro-6-[(tetrahydro-pyran-4-ylmethyl)-amino]-[2,4′]bipyridinyl-2′-yl}-amide; and
(R)-Piperidine-3-carboxylic acid {3-chloro-5′-fluoro-6-[(tetrahydro-pyran-4-ylmethyl)-amino]-[2,4′]bipyridinyl-2′-yl}-amide.

15. A compound of claim 1 selected from:
(3R,6R)-6-Methyl-piperidine-3-carboxylic acid {5′-chloro-6-[((R)-2,2-dimethyl-tetrahydro-pyran-4-ylmethyl)-amino]-[2,4′]bipyridinyl-2′-yl}-amide;
(3R,5S)-5-Trifluoromethyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Ethyl-piperidine-3-carboxylic acid {5′-chloro-6-[(tetrahydro-pyran-4-ylmethyl)-amino]-[2,4′]bipyridinyl-2′-yl}-amide;
(3R,5S)-5-Methyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Methyl-piperidine-3-carboxylic acid {5′-chloro-6-[(tetrahydro-pyran-4-ylmethyl)-amino]-[2,4′]bipyridinyl-2′-yl}-amide;
(3R,6R)-6-Methyl-piperidine-3-carboxylic acid {5′-chloro-6-[((S)-2,2-dimethyl-tetrahydro-pyran-4-ylmethyl)-amino]-[2,4′]bipyridinyl-2′-yl}-amide;
(3R,6R)-6-Methyl-piperidine-3-carboxylic acid {5′-chloro-5-fluoro-6-[(tetrahydro-pyran-4-ylmethyl)-amino]-[2,4′]bipyridinyl-2′-yl}-amide;
(3R,6S)-6-Methyl-piperidine-3-carboxylic acid {5′-chloro-6-[(tetrahydro-pyran-4-ylmethyl)-amino]-[2,4′]bipyridinyl-2′-yl}-amide; and
(3R,6R)-6-Ethyl-piperidine-3-carboxylic acid {5′-chloro-5-fluoro-6-[(tetrahydro-pyran-4-ylmethyl)-amino]-[2,4′]bipyridinyl-2′-yl}-amide.

16. A compound of claim 1 selected from:
(R)-Piperidine-3-carboxylic acid {5′-chloro-6-[(4-cyano-tetrahydro-pyran-4-ylmethyl)-amino]-[2,4′]bipyridinyl-2′-yl}-amide;
(R)-Piperidine-3-carboxylic acid {5′-chloro-6-[(4-methyl-tetrahydro-pyran-4-ylmethyl)-amino]-[2,4′]bipyridinyl-2′-yl}-amide;
(R)-Piperidine-3-carboxylic acid {5′-chloro-6-[(4-fluoro-tetrahydro-pyran-4-ylmethyl)-amino]-[2,4′]bipyridinyl-2′-yl}-amide;
(R)-Piperidine-3-carboxylic acid {5′-chloro-5-fluoro-6-[(4-methyl-tetrahydro-pyran-4-ylmethyl)-amino]-[2,4′]bipyridinyl-2′-yl}-amide;
(R)-Piperidine-3-carboxylic acid {3,5′-dichloro-6-[(4-methoxy-tetrahydro-pyran-4-ylmethyl)-amino]-[2,4′]bipyridinyl-2′-yl}-amide;
(R)-Piperidine-3-carboxylic acid {5′-chloro-5-fluoro-6-[(4-methoxy-tetrahydro-pyran-4-ylmethyl)-amino]-[2,4′]bipyridinyl-2′-yl}-amide; and
(R)-Piperidine-3-carboxylic acid {5′-chloro-6-[(4-ethyl-tetrahydro-pyran-4-ylmethyl)-amino]-5-fluoro-[2,4′]bipyridinyl-2′-yl}-amide.

17. A compound of claim 1 selected from:
(1S,3R)-3-Amino-cyclopentanecarboxylic acid {5′-chloro-6-[(tetrahydro-pyran-4-ylmethyl)-amino]-[2,4′]bipyridinyl-2′-yl}-amide;
(R)-Piperidine-3-carboxylic acid [5′-chloro-6-(3-fluoro-benzylamino)-[2,4′]bipyridinyl-2′-yl]-amide;
6-Oxo-piperidine-3-carboxylic acid {5′-chloro-6-[(tetrahydro-pyran-4-ylmethyl)-amino]-[2,4′]bipyridinyl-2′-yl}-amide;
(1S,3R)-3-Amino-cyclopentanecarboxylic acid {3,5′-dichloro-6-[(tetrahydro-pyran-4-ylmethyl)-amino]-[2,4′]bipyridinyl-2′-yl}-amide;
(1R,3R)-3-Amino-cyclopentanecarboxylic acid {5′-chloro-6-[(tetrahydro-pyran-4-ylmethyl)-amino]-[2,4′]bipyridinyl-2′-yl}-amide;
(1R,3S)-3-Amino-cyclopentanecarboxylic acid {3,5′-dichloro-6-[(tetrahydro-pyran-4-ylmethyl)-amino]-[2,4′]bipyridinyl-2′-yl}-amide;
(R)-Piperidine-3-carboxylic acid [5′-chloro-6-(3,5-difluoro-benzylamino)-[2,4′]bipyridinyl-2′-yl]-amide; and
(1R,3S)-3-Amino-cyclopentanecarboxylic acid {5′-chloro-6-[(tetrahydro-pyran-4-ylmethyl)-amino]-[2,4′]bipyridinyl-2′-yl}-amide.

18. A compound of claim 1 selected from:
(3R,5S)-5-Methoxymethyl-pyrrolidine-3-carboxylic acid {5′-chloro-5-fluoro-6-[(4-methoxy-tetrahydro-pyran-4-ylmethyl)-amino]-[2,4′]bipyridinyl-2′-yl}-amide;
(3R,5S)-5-Methoxymethyl-pyrrolidine-3-carboxylic acid {5′-chloro-6-[(4-methyl-tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-6-[(2,2-dimethyl-tetrahydro-pyran-4-ylmethyl)-amino]-[2,4′]bipyridinyl-2′-yl}-amide;
(3R,5S)-5-Methoxymethyl-pyrrolidine-3-carboxylic acid {3,5′-dichloro-6-[(2,2-dimethyl-tetrahydro-pyran-4-ylmethyl)-amino]-[2,4′]bipyridinyl-2′-yl}-amide;
(3S,4R)-4-Methoxy-pyrrolidine-3-carboxylic acid {3,5′-dichloro-5-fluoro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-6-[(tetrahydro-pyran-4-ylmethyl)-amino]-[2,4′]bipyridinyl-2′-yl}-amide;
(3R,5S)-5-Methoxymethyl-pyrrolidine-3-carboxylic acid {3,5′-dichloro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {3,5′-dichloro-6-[(tetrahydro-pyran-4-ylmethyl)-amino]-[2,4′]bipyridinyl-2′-yl}-amide;
(3S,4R)-4-Methoxy-pyrrolidine-3-carboxylic acid {5′-chloro-5-fluoro-6-[(tetrahydro-pyran-4-ylmethyl)-amino]-[2,4′]bipyridinyl-2′-yl}-amide; and
(3S,4R)-4-Methoxy-pyrrolidine-3-carboxylic acid {5′-chloro-6-[((2R,6S)-2,6-dimethyl-tetrahydro-pyran-4-ylmethyl)-amino]-[2,4′]bipyridinyl-2′-yl}-amide.

19. A compound of claim 1 selected from:
(R)-Morpholine-2-carboxylic acid {5′-chloro-5-fluoro-6-[(tetrahydro-pyran-4-ylmethyl)-amino]-[2,4′]bipyridinyl-2′-yl}-amide;
(S)-[1,4]Oxazepane-6-carboxylic acid {3,5′-dichloro-6-[(tetrahydro-pyran-4-ylmethyl)-amino]-[2,4′]bipyridinyl-2′-yl}-amide;
(R)-Morpholine-2-carboxylic acid {5′-chloro-3-fluoro-6-[(tetrahydro-pyran-4-ylmethyl)-amino]-[2,4′]bipyridinyl-2′-yl}-amide;
(R)-Morpholine-2-carboxylic acid {3,5′-dichloro-6-[(tetrahydro-pyran-4-ylmethyl)-amino]-[2,4′]bipyridinyl-2′-yl}-amide;
(R)-Morpholine-2-carboxylic acid {5′-chloro-6-[((R)-2,2-dimethyl-tetrahydro-pyran-4-ylmethyl)-amino]-[2,4′]bipyridinyl-2′-yl}-amide;
(R)-Morpholine-2-carboxylic acid {3,5′-dichloro-6-[((R)-2,2-dimethyl-tetrahydro-pyran-4-ylmethyl)-amino]-[2,4′]bipyridinyl-2′-yl}-amide;
(R)-Morpholine-2-carboxylic acid {3,5′-dichloro-6-[((S)-2,2-dimethyl-tetrahydro-pyran-4-ylmethyl)-amino]-[2,4′]bipyridinyl-2′-yl}-amide;
(R)-Morpholine-2-carboxylic acid {5′-chloro-6-[(tetrahydro-pyran-4-ylmethyl)-amino]-[2,4′]bipyridinyl-2′-yl}-amide; and
(R)-Morpholine-2-carboxylic acid {5′-chloro-6-[((S)-2,2-dimethyl-tetrahydro-pyran-4-ylmethyl)-amino]-[2,4′]bipyridinyl-2′-yl}-amide.

20. A compound according to any one of claims 12 to 19, or pharmaceutically acceptable salt thereof, for use in a method of treating a disease or condition mediated by CDK9.

21. The use of a compound according to any one of claims 12 to 19, or a pharmaceutically acceptable salt thereof, in the manufacture of a medicament for the treatment of a disease or condition mediated by CDK9.

22. A method of treatment of a disease or condition mediated by CDK9 comprising administration to a subject in need thereof a therapeutically effective amount of a compound according to any one of claims 12 to 19, or a pharmaceutically acceptable salt thereof.

23. A pharmaceutical composition comprising a compound according to any one of claims 12 to 19, or a pharmaceutically acceptable salt thereof, and a pharmaceutically acceptable carrier, diluent or excipient.

24. A compound of Formula II 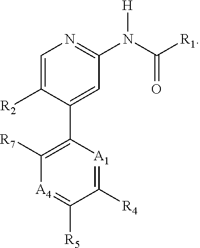
or a pharmaceutically acceptable salt thereof, wherein:
R₁ is selected from —(CH₂)₀₋₂-heteroaryl, —(CH₂)₀₋₂-aryl, C₁₋₈ alkyl, C₃₋₈ branched alkyl, C₃₋₈ cycloalkyl, and a 4 to 8 membered heterocycloalkyl group, wherein said groups are each independently optionally substituted;
R₂ is selected from hydrogen, C₁₋₄ alkoxy, C₁₋₄ haloalkyl, C₁₋₄-alkyl, and halogen;
A₁ is CR₃;
A₄ is N;
R₃ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;
R₄ is selected from hydrogen, halogen, 5 to 7 membered heterocyclyl-R¹⁴, and A₆-L-R₉;
R₅ is selected from hydrogen, C₁₋₄ alkyl, C₁₋₄ haloalkyl, hydroxyl, CN, —O—C₁₋₄ alkyl, —O—C₁₋₄ haloalkyl, C₃₋₄ cycloalkyl, C₃₋₄ cyclo haloalkyl, and halogen;
R₇ is selected from hydrogen, C₁₋₄ alkyl, C₁₋₄ haloalkyl, O—C₁₋₃ alkyl, and halogen;
A₆ is selected from O, SO₂, and NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;
R₈ is selected from hydrogen, C₁₋₄ alkyl, and C₃₋₈ branched-alkyl, and —C₃₋₈ branched haloalkyl;
R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted; and
R¹⁴ is selected from hydrogen, phenyl, halogen, hydroxy, C₁₋₄-alkyl, H, C₃₋₆-branched alkyl, C₁₋₄-haloalkyl, CF₃, ═O, and O—C₁₋₄-alkyl.

25. A compound of claim 24, wherein:
R₁ is selected from —(CH₂)₀₋₂-heteroaryl, —(CH₂)₀₋₂-aryl, wherein said groups are each independently optionally substituted with one to three substituents selected from —NH₂, —F, —Cl, —OH, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ cyclo haloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl, —C(O)—NR¹⁵R¹⁶, and —SO₂—NR¹⁵R¹⁶, and further wherein, any two said substituents along with the atoms to which they are attached can form a ring;
R₂ is selected from hydrogen, C₁₋₄ alkoxy, C₁₋₄ haloalkyl, C₁₋₄-alkyl, and halogen;
A₁ is CR₃;
A₄ is N;
R₃ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, —O—C₁₋₄ haloalkyl, and halogen;
R₄ is selected from hydrogen, halogen, 5 to 7 membered heterocyclyl-R¹⁴, or A₆-L-R₉;
R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;
R₇ is selected from hydrogen, C₁₋₄ alkyl, C₁₋₄ haloalkyl, O—C₁₋₃ alkyl, and halogen;
A₆ is O, SO₂, or NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene;
R₈ is selected from hydrogen, C₁₋₄ alkyl, and C₃₋₈ branched-alkyl, and —C₃₋₈ branched haloalkyl;
R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;
R¹⁴ is selected from hydrogen, phenyl, halogen, hydroxy, C₁₋₄-alkyl, H, C₃₋₆-branched alkyl, C₁₋₄-haloalkyl, CF₃, ═O, and O—C₁₋₄-alkyl; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

26. A compound of claim 24, wherein:
R₁ is selected from —(CH₂)₀₋₂-heteroaryl, and —(CH₂)₀₋₂-aryl, wherein said groups are each independently optionally substituted with one to three substituents selected from the group consisting of —NH₂, F, Cl, —OH, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, —O—(CH₂)₁₋₂—C₃₋₆ cycloalkyl-R¹⁴, —O—(CH₂)₁₋₂—C₄₋₆ heterocycloalkyl-R¹⁴, —SO₂—NR¹⁵R¹⁶, and —SO₂—C₃₋₅ cycloalkyl;
R₂ is selected from hydrogen, and halogen;
A₁ is CR₃;
A₄ is N;
R₃ is hydrogen;
R₄ is selected from piperidinyl, morpholinyl, pyrrolidinyl, and A₆-L-R₉; wherein each said piperidinyl, morpholinyl, pyrrolidinyl group is substituted with R¹⁴;
R₅ is selected from hydrogen, Cl, F, and CF₃;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and C₁₋₄ alkyl;
R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl;
R¹⁴ is selected from phenyl, halogen, hydroxyl, C₁₋₂-alkyl, CF₃, and hydrogen; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

27. A compound of claim 24, wherein:
R₁ is selected from C₁₋₈ alkyl, C₃₋₈ cycloalkyl, C₃₋₈ branched alkyl, and a 4 to 8 membered heterocycloalkyl group, wherein said groups are each independently optionally substituted with one to three substituents selected from —NH₂, —F, —OH, ═O, —C₁₋₄ alkyl, —C₁₋₄ haloalkyl, —C₃₋₆ branched alkyl, C₃₋₆ branched haloalkyl, —C₃₋₇ cyclo alkyl, —C₃₋₇ cyclo haloalkyl, —(CH₂)₁₋₃—O—C₁₋₂ alkyl, —(CH₂)₁₋₃—O—C₁₋₂ haloalkyl, —(CH₂)₀₋₂—O—(CH₂)₂₋₃—O—C₁₋₂ alkyl, —(CH₂)₀₋₂—O—(CH₂)₂₋₃—O—C₁₋₂ haloalkyl, —O—C₁₋₄ alkyl, —O—C₁₋₄ haloalkyl, —O—C₃₋₆ branched alkyl, —O—C₃₋₆ branched haloalkyl, —O—C₃₋₇ cyclo alkyl, —O—C₃₋₇ cyclo haloalkyl, —O—(CH₂)₁₋₂—C₃₋₆ cycloalkyl-R¹⁴, —O—(CH₂)₁₋₂—C₄₋₆ heterocycloalkyl-R¹⁴, —NH—C₁₋₄ alkyl, —NH—C₂₋₄ haloalkyl, —NH—C₃₋₈ branched alkyl, —NH—C₃₋₈ branched haloalkyl, —NH—C₃₋₇ cyclo alkyl, —NH—C₃₋₇ cyclo haloalkyl, —NH—C(O)—C₁₋₄ alkyl, —NH—C(O)—C₁₋₄ haloalkyl, —NH—C(O)—C₃₋₈ branched alkyl, —NH—C(O)—C₃₋₈ branched haloalkyl, —NH—C(O)—C₃₋₇ cyclo alkyl, —NH—C(O)—C₃₋₇ cyclo haloalkyl, —NH—C(O)—CH₂—O—C₁₋₄ alkyl, —NH—C(O)—CH₂—O—C₁₋₄ haloalkyl, —NH—C(O)—O—C₁₋₄ alkyl, —NH—C(O)O—C₂₋₄ haloalkyl, —NH—C(O)—O—C₃₋₈ branched alkyl, —NH—C(O)O—C₃₋₈ branched haloalkyl, —NH—C(O)—O—C₃₋₇ cyclo alkyl, —NH—C(O)—O—C₃₋₇ cyclo haloalkyl, —NH—SO₂—C₁₋₄ alkyl, —NH—SO₂—C₁₋₄ haloalkyl, —NH—SO₂—C₃₋₈ branched alkyl, —NH—SO₂—C₃₋₈ branched haloalkyl, —NH—SO₂—C₃₋₅ cycloalkyl, —NH—SO₂—C₃₋₅ halo-cycloalkyl, —C(O)—O—C₁₋₄ alkyl, —C(O)—O—C₂₋₄ halo-alky, —C(O)—O—C₃₋₆ branched alkyl, —C(O)O—C₃₋₆ branched haloalkyl, —C(O)—O—C₃₋₇ cyclo alkyl, —NH—C(O)—O—C₃₋₇ cyclo haloalkyl, —C(O)—C₁₋₄ alkyl, —C(O)C₂₋₄ haloalkyl, —C(O)—C₃₋₈ branched alkyl, —C(O)—C₃₋₈ branched haloalkyl, —C(O)—C₃₋₇ cyclo alkyl, —NH—C(O)—O—C₃₋₇ cyclo haloalkyl, —C(O)—CH₂—O—C₁₋₄ alkyl, —C(O)—CH₂—O—C₁₋₄ haloalkyl, —SO₂—C₁₋₄ alkyl, —SO₂—C₁₋₄ haloalkyl, —SO₂—C₃₋₈ branched alkyl, —SO₂—C₃₋₈ branched haloalkyl, —SO₂—C₃₋₅ cycloalkyl, and —SO₂—C₃₋₅ cyclo haloalkyl; —C(O)—NR¹⁵R¹⁶, and —SO₂—NR¹⁵R¹⁶, and further wherein, any two said substituents along with the atoms to which they are attached can form a ring;
R₂ is selected from hydrogen, C₁₋₄ alkoxy, C₁₋₄ haloalkyl, C₁₋₄-alkyl, and halogen;
A₁ is CR₃;
A₄ is N;
R₃ is selected from hydrogen, C₁₋₄ alkyl, C₁₋₄ haloalkyl, CN, —O—C₁₋₄ alkyl, C₃₋₄ cycloalkyl, C₃₋₄ cyclo haloalkyl, and halogen;
R₄ is selected from hydrogen, halogen, 5 to 7 membered heterocyclyl-R¹⁴, and A₆-L-R₉;
R₅ is selected from hydrogen, C₁₋₄ alkyl, C₁₋₄ haloalkyl, CN, —O—C₁₋₄ alkyl, —O—C₁₋₄ haloalkyl, C₃₋₄ cycloalkyl, C₃₋₄ cyclo haloalkyl, and halogen;
R₇ is selected from hydrogen, C₁₋₄ alkyl, C₁₋₄ haloalkyl, O—C₁₋₃ alkyl, and halogen;
A₆ is selected from O, SO₂, and NR₈;
L is selected from C₀₋₃-alkylene, —CHD-, —CD₂-, C₃₋₆ cycloalkyl, C₃₋₆ cyclo haloalkyl, C₄₋₇-heterocycloalkyl, C₃₋₈ branched alkylene, C₃₋₈ branched haloalkylene;
R₈ is selected from hydrogen, C₁₋₄ alkyl, and C₃₋₈ branched-alkyl, and —C₃₋₈ branched haloalkyl;
R₉ is selected from hydrogen, C₁₋₆ alkyl, C₃₋₈ cycloalkyl, C₃₋₈ branched alkyl, —(CH₂)₀₋₂ heteroaryl, (CH₂)₀₋₂-4 to 8 member heterocycloalkyl, and (CH₂)₀₋₂-aryl, wherein said groups are optionally substituted;
R¹⁴ is selected from hydrogen, phenyl, halogen, hydroxy, C₁₋₄-alkyl, H, C₃₋₆-branched alkyl, C₁₋₄-haloalkyl, CF₃, ═O, and O—C₁₋₄-alkyl; and
R¹⁵ and R¹⁶ are independently selected from hydrogen, hydroxyl, alkyl, branched alkyl, haloalkyl, branched haloalkyl, alkoxy, cycloalkyl and heterocycloalkyl; and alternatively, R¹⁵ and R¹⁶ along with the nitrogen atom to which they are attached to can be taken together to form an optionally substituted four to six membered heteroaromatic, or non-aromatic heterocyclic ring.

28. A compound of claim 24, wherein:
R₁ is selected from C₁₋₈ alkyl, C₃₋₈ branched alkyl, C₃₋₈ cycloalkyl, and a 4 to 8 membered heterocycloalkyl group, wherein said groups are each independently optionally substituted with one to three substituents selected from the group consisting of —NH₂, F, —OH, ═O, —C₁₋₄ alkyl, —NH—C₁₋₄ alkyl, —C₁₋₄ haloalkyl, —C₃₋₆ branched alkyl, —(CH₂)₁₋₃—O—C₁₋₂ alkyl, —NH—C(O)—CH₂—O—C₁₋₄ alkyl, —NH—C(O)—C₁₋₄ alkyl, —NH—C(O)—C₃₋₈ branched alkyl, —O—C₃₋₆ branched alkyl, —NH—C(O)—O—C₁₋₄ alkyl, —NH—SO₂—C₁₋₄ alkyl, —NH—SO₂—C₃₋₈ branched alkyl, —NH—SO₂—C₃₋₅ cycloalkyl, (CH₂)₀₋₂—O—(CH₂)₂₋₃—O—C₁₋₂ alkyl, —O—C₁₋₄ alkyl, —C(O)O—C₃₋₆ branched alkyl, —C(O)C₁₋₄ alkyl, —C(O)—O—C₁₋₄ alkyl, —C(O)—C₃₋₈ branched alkyl, —C(O)—CH₂—O—C₁₋₄ alkyl, —SO₂—C₁₋₄ alkyl, —SO₂—C₃₋₈ branched alkyl, and —SO₂—C₃₋₅ cycloalkyl;
R₂ is selected from hydrogen, and halogen;
A₁ is CR₃;
A₄ is N;
R₃ is hydrogen;
R₄ is selected from piperidinyl, morpholinyl, pyrrolidinyl, and A₆-L-R₉; wherein each said piperidinyl, morpholinyl, pyrrolidinyl group is substituted with R¹⁴;
R₅ is selected from hydrogen, Cl, F, and CF₃;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and C₁₋₄ alkyl;
R₉ is selected from C₁₋₃ alkyl, C₃₋₇ cycloalkyl, C₄₋₆ branched alkyl, —(CH₂)₁₋₃—O—C₁₋₄ alkyl, —(CH₂)-pyridyl, (CH₂)-4 to 8 member heterocycloalkyl, (CH₂)-4 to 8 member heterocycloalkyl, and (CH₂)-phenyl, wherein said groups are optionally substituted with one to three substituents selected from hydrogen, halogen, C₁₋₄ alkyl, C₁₋₄ haloalkyl, —OH, CN, ═O, C(O)—CH₃, —O—C₁₋₃ alkyl, —O—C₁₋₃ haloalkyl, —O—(CH₂)₂₋₃—O—C₁₋₂ alkyl, —C(O)—C₁₋₄ alkyl, and —NH—C(O)—C₁₋₄ alkyl; and
R¹⁴ is selected from phenyl, halogen, hydroxy, C₁₋₂-alkyl, and hydrogen.

29. A compound of claim 24, wherein:
R₁ is selected from piperidinyl, morpholinyl, 1-methylpiperidinyl, tetrahydro-pyran, pyrrolidinyl, tetrahydro-furan, azetidine, pyrrolidin-2-one, azepane, and 1,4-oxazepane, wherein said R₁ groups are each independently optionally substituted with one to three substituents selected from F, OH, NH₂, CO-methyl, —NH-methyl, ethyl, fluoro-ethyl, trifluoro-ethyl, (CH₂)₂-methoxy, SO₂—CH₃, COO—CH₃, SO₂-ethyl, SO₂-cyclopropyl, methyl, SO₂—CH—(CH₃)₂, NH—SO₂—CH₃, NH—SO₂—C₂H₅, ═O, CF₃, (CH₂)-methoxy, methoxy, NH—SO₂—CH—(CH₃)₂, —(CH₂)—O—(CH₂)₂-methoxy, —O—CH—(CH₃)₂;
R₂ is selected from Cl, and F;
A₁ is CR₃,
A₄ is N;
R₃ is hydrogen;
R₄ is A₆-L-R₉;
R₅ is selected from Cl, F, and hydrogen;
R₆ is H;
R₇ is selected from hydrogen, F, and Cl;
A₆ is NR₈;
L is selected from C₀₋₃-alkylene, —CD₂-, and C₃₋₈ branched alkylene;
R₈ is selected from hydrogen, and methyl; and
R₉ is selected from C₁₋₃ alkyl, C₄₋₆ branched alkyl, —(CH₂)₁₋₃—O—C₁₋₄ alkyl, —(CH₂)-pyridyl, benzyl, CD₂-tetrahydro-pyran, tetrahydro-pyran, tetrahydro-thiopyran 1,1-dioxide, piperidinyl, pyrrolidine-2-one, dioxane, cyclopropyl, tetrahydrofuran, cyclohexyl, and cycloheptyl, wherein said groups are optionally substituted with one to three substituents each independently selected from F, OCHF₂, CO-methyl, OH, methyl, methoxy, CN, ethyl, and NH—CO-methyl.

30. A compound of claim 24, wherein:
R₁ is selected from piperidinyl, morpholinyl, pyrrolidinyl, azepane, and 1,4-oxazepane, wherein said R₁ groups are each independently optionally substituted with one to three substituents selected from F, methyl, CF₃, ethyl, fluoro-ethyl, trifluoro-ethyl, —(CH₂)₂-methoxy, —(CH₂)-methoxy, methoxy, ═O, —(CH₂)—O—(CH₂)₂-methoxy, —O—CH—(CH₃)₂;
R₂ is Cl;
A₁ is CR₃;
A₄ is N;
R₃ is hydrogen;
R₄ is A₆-L-R₉;
R₅ is selected from Cl, F, and hydrogen;
R₆ is H;
R₇ is selected from Cl, F, and hydrogen;
A₆ is NR₈;
L is selected from —CH₂—, —CD₂-;
R₈ is selected from hydrogen, and methyl; and
R₉ is selected from pyridyl, benzyl, tetrahydro-pyran, dioxane, tetrahydrofuran, wherein said groups are optionally substituted with one to three substituents each independently selected from F, OH, methyl, ethyl, methoxy, CN.

31. A compound of claim 24 selected from:
(R)-Piperidine-3-carboxylic acid {2,5′-dichloro-5-[(tetrahydro-pyran-4-ylmethyl)-amino]-[3,4′]bipyridinyl-2′-yl}-amide;
(R)-Piperidine-3-carboxylic acid {6,5′-dichloro-5-[(tetrahydro-pyran-4-ylmethyl)-amino]-[3,4′]bipyridinyl-2′-yl}-amide; and
(R)-Piperidine-3-carboxylic acid {5′-chloro-5-[(tetrahydro-pyran-4-ylmethyl)-amino]-[3,4′]bipyridinyl-2′-yl}-amide.

32. A compound according to any one of claims 30 to 31, or pharmaceutically acceptable salt thereof, for use in a method of treating a disease or condition mediated by CDK9.

33. The use of a compound according to any one of claims 30 to 31, or a pharmaceutically acceptable salt thereof, in the manufacture of a medicament for the treatment of a disease or condition mediated by CDK9.

34. A method of treatment of a disease or condition mediated by CDK9 comprising administration to a subject in need thereof a therapeutically effective amount of a compound according to any one of claims 30 to 31, or a pharmaceutically acceptable salt thereof.

35. A pharmaceutical composition comprising a compound according to any one of claims 30 to 31, or a pharmaceutically acceptable salt thereof, and a pharmaceutically acceptable carrier, diluent or excipient.
